(19) United States
(12) Patent Application Publication
(10) Pub. No.: US 2023/0357436 A1
(43) Pub. Date: Nov. 9, 2023

(54) ANTI-IDIOTYPE COMPOSITIONS AND METHODS OF USE THEREOF

(71) Applicant: Exuma Biotech Corp., West Palm Beach, FL (US)

(72) Inventors: Gregory Ian FROST, West Palm Beach, FL (US); Gregory Harold SCHREIBER, Palm Beach Gardens, FL (US); Anirban KUNDU, George Town, Grand Cayman, KY (US)

(73) Assignee: Exuma Biotech Corp., West Palm Beach, FL (US)

(21) Appl. No.: 18/043,465

(22) PCT Filed: Aug. 31, 2021
(86) PCT No.: PCT/US2021/048532

§ 371 Date: Feb. 28, 2023

Related U.S. Application Data
Application No. PCT/US2021/048532 is a continuation-in-part of application No. PCT/US2021/020922, filed on Mar. 4, 2021.
Application No. PCT/US2021/048532 is a continuation-in-part of application No. PCT/US2020/048843, filed on Aug. 31, 2020.
(63) Continuation-in-part of application No. PCT/US2020/048843, filed on Aug. 31, 2020.
(63) Continuation-in-part of application No. PCT/US2019/049259, filed on Sep. 2, 2019.
(63) Continuation-in-part of application No. PCT/US2018/051392, filed on Sep. 17, 2018.
(63) Continuation-in-part of application No. PCT/US2018/020818, filed on Mar. 3, 2018.
(63) Continuation-in-part of application No. 15/644,778, filed on Jul. 8, 2017, now Pat. No. 11,111,505.
(63) Continuation-in-part of application No. PCT/US2017/041277, filed on Jul. 8, 2017.
(63) Continuation-in-part of application No. 15/462,855, filed on Mar. 19, 2017, now Pat. No. 10,596,274.
(63) Continuation-in-part of application No. PCT/US2017/023112, filed on Mar. 19, 2017.
Application No. 15/644,778 is a continuation-in-part of application No. 15/462,855, filed on Mar. 19, 2017, now Pat. No. 10,596,274.
(60) Provisional application No. 63/200,329, filed on Mar. 1, 2021.
(60) Provisional application No. 63/200,329, filed on Mar. 1, 2021.
(60) Provisional application No. 63/136,177, filed on Jan. 11, 2021.
(60) Provisional application No. 63/136,177, filed on Jan. 11, 2021.
(60) Provisional application No. 62/985,741, filed on Mar. 5, 2020.
(60) Provisional application No. 62/985,741, filed on Mar. 5, 2020.
(60) Provisional application No. 62/943,207, filed on Dec. 3, 2019.
(60) Provisional application No. 62/894,926, filed on Sep. 2, 2019.
(60) Provisional application No. 62/894,853, filed on Sep. 1, 2019.
(60) Provisional application No. 62/894,852, filed on Sep. 1, 2019.
(60) Provisional application No. 62/894,849, filed on Sep. 1, 2019.
(60) Provisional application No. 62/821,434, filed on Mar. 20, 2019.
(60) Provisional application No. 62/732,528, filed on Sep. 17, 2018.
(60) Provisional application No. 62/728,056, filed on Sep. 6, 2018.
(60) Provisional application No. 62/728,056, filed on Sep. 6, 2018.
(60) Provisional application No. 62/726,294, filed on Sep. 2, 2018.
(60) Provisional application No. 62/726,294, filed on Sep. 2, 2018.
(60) Provisional application No. 62/726,293, filed on Sep. 2, 2018.
(60) Provisional application No. 62/726,293, filed on Sep. 2, 2018.
(60) Provisional application No. 62/564,991, filed on Sep. 28, 2017.
(60) Provisional application No. 62/564,991, filed on Sep. 28, 2017.
(60) Provisional application No. 62/564,253, filed on Sep. 27, 2017.
(60) Provisional application No. 62/564,253, filed on Sep. 27, 2017.
(60) Provisional application No. 62/560,176, filed on Sep. 18, 2017.
(60) Provisional application No. 62/560,176, filed on Sep. 18, 2017.
(60) Provisional application No. 62/467,039, filed on Mar. 3, 2017.
(60) Provisional application No. 62/467,039, filed on Mar. 3, 2017.
(60) Provisional application No. 62/467,039, filed on Mar. 3, 2017.
(60) Provisional application No. 62/467,039, filed on Mar. 3, 2017.
(60) Provisional application No. 62/467,039, filed on Mar. 3, 2017.
(60) Provisional application No. 62/467,039, filed on Mar. 3, 2017.
(60) Provisional application No. 62/360,041, filed on Jul. 8, 2016.
(60) Provisional application No. 62/360,041, filed on Jul. 8, 2016.
(60) Provisional application No. 62/360,041, filed on Jul. 8, 2016.
(60) Provisional application No. 62/360,041, filed on Jul. 8, 2016.
(60) Provisional application No. 62/360,041, filed on Jul. 8, 2016.
(60) Provisional application No. 62/390,093, filed on Mar. 19, 2016.
(60) Provisional application No. 62/390,093, filed on Mar. 19, 2016.
(60) Provisional application No. 62/390,093, filed on Mar. 19, 2016.
(60) Provisional application No. 62/390,093, filed on Mar. 19, 2016.

(51) Int. Cl. A61K 47/69; A61K 47/62; C12N 15/113; C07K 16/28; C07K 14/725; C07K 16/42; C12N 15/86; C12N 9/64; A61K 39/00

(52) U.S. Cl. CPC C07K 16/4258; A61K 39/4611; A61K 39/4613; A61K 39/4637; A61K 39/4643; A61K 47/62; A61K 47/6901; C07K 14/7051; C07K 16/2803; C07K 16/2809; C07K 16/2863; C12N 9/6472; C12N 15/1136; C12N 15/86; C12Y 304/22055; A61K 2239/21; A61K 2239/22; A61K 2239/29; C07K 2317/31; C07K 2317/567; C07K 2317/622; C07K 2317/73; C07K 2317/76; C07K 2319/02; C07K 2319/03; C07K 2319/33; C07K 2319/50; C07K 2319/75; C12N 2310/14; C12N 2740/15043; C12N 2810/852; C12N 2840/002; C12N 2840/007; C12N 2840/203

(57) ABSTRACT

The present disclosure provides methods and compositions that include a polynucleotide that includes nucleic acids that encode an anti-idiotype polypeptide, as well as polypeptides that are encoded by the same, and cells that include and express the polypeptide. Disclosed methods include methods that utilize the anti-idiotype polypeptides as safety switches when they are used in combination with antibodies, include approved biologic antibodies, that include the recognized idiotype. Certain embodiments include anti-idiotype polypeptides and nucleotides encoding the same, that include an internal domain. This internal domain in some embodiments has functional domains that can induce proliferation or cell death upon binding of the anti-idiotype polypeptide by its target antibody.

### CROSS-REFERENCE TO RELATED APPLICATIONS

This application claims priority to International Application No. PCT/US2021/020922, filed Mar. 4, 2021; International Application No. PCT/US2020/048843, filed Aug. 31, 2020; U.S. Provisional Application No. 63/200,329, filed Mar. 1, 2021; and U.S. Provisional Application No. 63/136,177, filed Jan. 11, 2021; International Application No. PCT/US2021/020922, filed Mar. 4, 2021 claims priority to U.S. Provisional Application No. 62/985,741, filed Mar. 5, 2020; International Application No. PCT/US2020/048843, filed Aug. 31, 2020; U.S. Provisional Application No. 63/136,177, filed Jan. 11, 2021; and U.S. Provisional Application No. 63/200,329, filed Mar. 1, 2021; International Application No. PCT/US2020/048843, filed Aug. 31, 2020 is a continuation-in-part of International Application No. PCT/US2019/049259, filed Sep. 2, 2019; and claims the benefit of U.S. Provisional Application No. 62/894,849, filed Sep. 1, 2019; U.S. Provisional Application No. 62/894,852, filed Sep. 1, 2019; U.S. Provisional Application No. 62/894,853, filed Sep. 1, 2019; U.S. Provisional Application No. 62/894,926, filed Sep. 2, 2019; U.S. Provisional Application No. 62/943,207, filed Dec. 3, 2019; and U.S. Provisional Application No. 62/985,741, filed Mar. 5, 2020; International Application No. PCT/US2019/049259 is a continuation-in-part of International Application No. PCT/US2018/051392 filed Sep. 17, 2018; and claims the benefit of U.S. Provisional Application No. 62/726,293, filed Sep. 2, 2018; U.S. Provisional Application No. 62/726,294, filed Sep. 2, 2018; U.S. Provisional Application No. 62/728,056 filed Sep. 6, 2018; U.S. Provisional Application No. 62/732,528, filed Sep. 17, 2018; U.S. Provisional Application No. 62/821,434, filed Mar. 20, 2019; and U.S. Provisional Application No. 62/894,853, filed Sep. 1, 2019; International Application No. PCT/US2018/051392 is a continuation-in-part of International Application No. PCT/US2018/020818, filed Mar. 3, 2018; and claims the benefit of U.S. Provisional Application No. 62/560,176, filed Sep. 18, 2017; U.S. Provisional Application No. 62/564,253, filed Sep. 27, 2017; U.S. Provisional Application No. 62/564,991, filed Sep. 28, 2017; and U.S. Provisional Application No. 62/728,056, filed Sep. 6, 2018; International Application No. PCT/US2018/020818 is a continuation-in-part of International Application No. PCT/US2017/023112 filed Mar. 19, 2017; a continuation-in-part of International Application No. PCT/US2017/041277 filed Jul. 8, 2017; a continuation-in-part of U.S. Application No. 15/462,855 filed Mar. 19, 2017; and a continuation-in-part of U.S. Application No. 15/644,778 filed Jul. 8, 2017; and claims the benefit of U.S. Provisional Application No. 62/467,039 filed Mar. 3, 2017; U.S. Provisional Application No. 62/560,176 filed Sep. 18, 2017; U.S. Provisional Application No. 62/564,253 filed Sep. 27, 2017; and U.S. Provisional Application No. 62/564,991 filed Sep. 28, 2017; International Application No. PCT/US2017/023112 claims the benefit of U.S. Provisional Application No. 62/390,093, filed Mar. 19, 2016; U.S. Provisional Application No. 62/360,041, filed Jul. 8, 2016; and U.S. Provisional Application No. 62/467,039, filed Mar. 3, 2017; International Application No. PCT/US2017/041277 claims the benefit of International Application No. PCT/US2017/023112, filed Mar. 19, 2017; U.S. Pat. Application No. 15/462,855, filed Mar. 19, 2017; U.S. Provisional Application No. 62/360,041, filed Jul. 8, 2016; and U.S. Provisional Application No. 62/467,039, filed Mar. 3, 2017; U.S. Application No. 15/462,855 claims the benefit of U.S. Provisional Application No. 62/390,093, filed Mar. 19, 2016; U.S. Provisional Application No. 62/360,041, filed Jul. 8, 2016; and U.S. Provisional Application No. 62/467,039, filed Mar. 3, 2017; U.S. Application No. 15/644,778 is a continuation-in-part of International Application No. PCT/US2017/023112, filed Mar. 19, 2017; and a continuation-in-part of U.S. Pat. Application No. 15/462,855, filed Mar. 19, 2017; and claims the benefit of U.S. Provisional Application No. 62/360,041, filed Jul. 8, 2016, and U.S. Provisional Application No. 62/467,039, filed Mar. 3, 2017. These applications are incorporated by reference herein in their entireties.

### SEQUENCE LISTING

This application hereby incorporates by reference the material of the electronic Sequencing Listing filed concurrently herewith. The materials in the electronic Sequence Listing is submitted as a text (.txt) file entitled “F1_007_WO_01_Sequence_Listing” created on Aug. 31, 2021, which has a file size of 729 KB, and is herein incorporated by reference in its entirety.

### FIELD OF INVENTION

This disclosure relates to the field of immunology, or more specifically, to the genetic modification of T lymphocytes or other immune cells, and methods of controlling proliferation of such cells.

### BACKGROUND OF THE DISLOSURE

Lymphocytes from a subject (e.g., patient) can be genetically modified ex vivo or in vivo to express synthetic proteins that enable redirected engagement with other cells and environments based upon the genetic programs incorporated. Examples of such synthetic proteins include engineered T cell receptors (TCRs) and chimeric antigen receptors (CARs). Such lymphocytes have become important therapeutics (e.g. CAR-T therapy) for treating diseases such as cancer.

Although efficacy for some cell therapies has been impressive and even curative for some patients and some cancers, safety remains a concern for therapies that use genetically modified cells, such as lymphocytes. For example, there is a risk that genetically modified cells introduced into a subject can become oncogenic for example due to the insertional site of a transgene within a target cell’s genome. For CAR-T, safety concerns arise for example because CAR-T cells can bind their target at sites other than tumors (i.e. on-target, off-tumor), thus leading to undesirable side effects. Furthermore, on-target and on-tumor toxicities can arise such as tumor lysis syndrome, cytokine release syndrome, and macrophage activation syndrome. Some of the adverse events of CAR-T cells can furthermore include painful and/or dangerous neurotoxicity including IEC-associated neurotoxicity syndrome (ICANS).

Accordingly, genetic safety switches have been developed to attempt to address these safety issues, by providing a means to kill genetically modified cells in a patient. However, these safety switches are time-consuming to develop and have limited design flexibility. For example, some safety switches require modifying a natural target for an approved antibody biologic, that is still capable of binding the antibody specifically. For example, previously disclosed safety switches include cell surface molecules that are truncated tyrosine kinase receptors. In some of these examples, the truncated tyrosine kinase receptor is a member of the epidermal growth factor receptor (EGFR) family (e.g., ErbB1 (HER1), ErbB2, ErbB3, and ErbB4), for example as disclosed in U.S. Pat. 8,802,374 or WO2018226897. Thus, some of these safety switches are polypeptides that are recognized by an antibody that recognizes the extracellular domain of an EGFR member. Such truncated EGFR polypeptides are sometimes referred to herein as eTags. An eTag was demonstrated to have cell killing potential through Erbitux® mediated antibody dependent cellular cytotoxicity (ADCC) pathways.

There are a number of drawbacks with current safety switches, and they fail to meet a number of long-standing challenges for cell therapy. One drawback with eTags is that they include EGFR family member domains, and thus are limited in structure to only EGFR domains. Furthermore, they may yield undesirable side effects when delivered to a subject since they are structurally related to growth factors (e.g. EGFR) found in a subject. Thus, there remains a need for a more flexible system to generate safety switches, that can utilize virtually any antibody, including virtually any approved antibody biologic, that can be constructed using established technologies, and that does not require delivery of modified growth factor receptors extracellular domains. Furthermore, there remains a long-standing challenge in cell therapies that use genetically modified cells, such as CAR-T, to provide genetic tools that are flexible and relatively easy to develop, that can be used to modify virtually any activity of the cell, including, but not limited to, inducing proliferation or cell death of the genetically modified cells.

### SUMMARY

Provided herein are methods, uses, compositions, and kits that help overcome issues related to prior art safety switches and to the effectiveness and safety of methods for performing cell therapy. The anti-idiotype polypeptide safety switches provided herein, are much more robust and flexible safety switches than prior art safety switches, such as eTags, in terms of design and development. Anti-idiotype polypeptide safety switches can be designed to be recognized by virtually any antibody, including any clinical antibody, such as a therapeutic antibody. Exemplary methods for developing anti-idiotype antibodies, are known.

Accordingly, in some aspects, provided herein are methods, compositions, and kits that are, or include or encode, anti-idiotype polypeptides. Furthermore, provided herein are methods for delivering modified lymphocytes, especially modified and genetically modified T cell and/or NK cells, and/or for regulating the activity of transduced, genetically modified, and/or modified T cells and/or NK cells. Such methods, compositions, and kits provide improved efficacy and safety over current technologies, especially with respect to T cells and/or NK cells that express lymphoproliferative elements (e.g., chimeric cytokine receptors), engineered T cell receptors (TCRs), and chimeric antigen receptors (CARs), including microenvironment restricted biologic (“MRB”) CARs. Transduced and/or modified and in illustrative embodiments genetically modified T cells and/or NK cells that are produced by and/or used in methods provided herein, include anti-idiotype functionality typically, in combination with other functionality, in illustrative embodiments delivered from retroviral (e.g., lentiviral) genomes via retroviral (e.g., lentiviral) particles. The anti-idiotype polypeptides, or polynucleotides encoding the same, alone or in combination with other functionality, provide improved features for such cells, including safety features and for methods that utilize such cells, such as research methods, commercial production methods, and adoptive cellular therapy. For example, such cells can be controllably killed if they become safety concerns to subjects in which they are delivered, and can have improved growth properties that can be better regulated.

In one aspect, provided herein is a polynucleotide, comprising one or more transcriptional units, wherein each of the one or more transcriptional units is operatively linked to a promoter wherein the one or more transcriptional units comprise:
a) a polynucleotide sequence encoding one or more inhibitory RNA molecules and/or a first engineered signaling polypeptide, and
b) a polynucleotide sequence encoding an anti-idiotype polypeptide comprising an extracellular recognition domain and a membrane association domain, wherein the extracellular recognition domain comprises a domain that recognizes an idiotype of a target antibody or a target antibody mimetic.

In one aspect, provided herein is a method for administering a cell therapy to a mammalian subject, said method comprising administering modified cells to the mammalian subject, wherein the modified cells each comprise a polynucleotide encoding: a) one or more inhibitory RNA molecules and/or a first engineered polypeptide, and b) an anti-idiotype polypeptide comprising an extracellular recognition domain and a membrane association domain, wherein the extracellular recognition domain comprises a domain that recognizes an idiotype of a target antibody or a target antibody mimetic.

In one aspect, provided herein is a method for administering a gene therapy to a mammalian subject, said method comprising administering gene vectors to the mammalian subject, wherein the gene vectors each comprise a polynucleotide encoding: a) one or more inhibitory RNA molecules and/or a first engineered polypeptide, and b) an anti-idiotype polypeptide comprising an extracellular recognition domain and a membrane association domain, wherein the extracellular recognition domain comprises a domain that recognizes an idiotype of a target antibody or a target antibody mimetic.

In some embodiments, the anti-idiotype polypeptide is capable of binding to an idiotype of a clinical stage or approved target antibody or antibody mimetic. Such target antibody or antibody mimetic can be capable of, adapted for, configured to, and/or effective for promoting cell death.

In some embodiments, the anti-idiotype polypeptide further include one or more intracellular domains. The intracellular signaling domains can activate or inhibit pro-apoptotic or anti-apoptotic pathways and/or pro-survival or anti-survival pathways.

Further details regarding aspects and embodiments of the present disclosure are provided throughout this patent application. Sections and section headers are for ease of reading and are not intended to limit combinations of disclosure, such as methods, compositions, and kits or functional elements therein across sections.

### BRIEF DESCRIPTION OF THE DRAWINGS

FIG. 1 shows a non-limiting example of a polynucleotide that is a portion of a lentiviral vector, with nucleic acids that encode an anti-idiotype polypeptide includes an anti-idiotype extracellular recognition domain (“Anti-idiotype”), a stalk, and a membrane association domain (MAD). The vector also includes a promoter (“Promoter”) positioned to drive expression of nucleic acids encoding different functional polypeptides including the anti-idiotype polypeptide, and includes various functional domains as indicated, including various viral functional domains, and encodes an inhibitory RNA molecule within the promoter.

FIGS. 2A-G show non-limiting examples of polynucleotides that include nucleic acids that encode one or more anti-idiotype polypeptides in various combinations with other polypeptides. FIG. 2A shows nucleic acids that encode an anti-idiotype polypeptide with a membrane association domain (MAD) and an anti-idiotype extracellular recognition domain (referred to as “anti-idiotype” in FIGS. 2). FIGS. 2B-D show an anti-idiotype polypeptide expressed as part of a single polynucleotide that also encodes a chimeric antigen receptor (CAR) or an engineered T cell receptor (TCR) (FIG. 2B), a lymphoproliferative element (FIG. 2C), or a cytokine (FIG. 2D). FIGS. 2E-G shows an anti-idiotype polypeptide is expressed as part of a single polynucleotide that also encodes a CAR or a TCR, an anti-idiotype polypeptide, along with a lymphoproliferative element (FIG. 2E), or a cytokine (FIG. 2F), or both a lymphoproliferative element and a cytokine (FIG. 2G).

FIGS. 3A-G show non-limiting examples of anti-idiotype polypeptides. FIG. 3A shows an anti-idiotype polypeptide with an extracellular recognition domain (sometimes referred to as an anti-idiotype extracellular recognition domain, anti-id ERD, or anti-id ECD, and denoted in FIGS. 3A-F and FIG. 3H as “anti-idiotype”), a stalk, and a membrane association domain (MAD). FIGS. 3B-3G show anti-idiotype polypeptides with one or more intracellular domains (sometimes referred to herein as ICDs) and a transmembrane domain (“TM” in FIGS. 3B-3G). FIG. 3B shows the anti-idiotype polypeptide with a single intracellular domain. FIGS. 3C-E shows the anti-idiotype polypeptide with either the P3 domain, and optionally a P4 domain, of a lymphoproliferative element (FIG. 3C), or with the intracellular domain of a CAR or TCR (FIG. 3D), or with the intracellular domain of a death domain. FIG. 3F shows an anti-idiotype polypeptide with the anti-id ERD as one of the ASTRs of a bi-specific CAR or TCR. FIG. 3G shows an anti-idiotype polypeptide similar to FIG. 3B, but with a cleavage site between or as part of the transmembrane domain and the ICD.

FIGS. 4 shows show non-limiting examples of anti-idiotype polypeptides.

FIGS. 5A and 5B show schematics of illustrative bicistronic lentiviral genomic vectors with divergent transcriptional units.

FIG. 6A shows the panning round, phage input (pfu), bead volume, binding time, number of washes, phage output (pfu) recovery rate, and enrichment for Panning Experiment 1 using Dynabeads™ Protein A.

FIG. 6B shows the panning round, phage input (pfu), bead volume, binding time, number of washes, phage output (pfu) recovery rate, and enrichment for Panning Experiment 1 using Dynabeads™ M-270 Epoxy

FIG. 7 shows the binding of the phage output from rounds 1-5 of Panning Experiment 1 to cetuximab as measured by ELISA. Phage were eluted from Dynabeads™ M-270 Epoxy using Elution Buffer in each panning round except as indicated for R5 in which bound phage were eluted with EGFR.

FIG. 8 shows sequence information for the 19 unique clones identified in Panning Experiment 1 using Ab1.1 as bait

FIGS. 9A-9C shows sequence information for the 42 unique clones identified in Panning Experiment 2 using Ab1.2 as bait

FIG. 10 shows the results of a competitive binding assay in which soluble EGFR competed with monoclonal phage clones from Panning Experiment 2 for binding to cetuximab by ELISA.

FIG. 11 shows a schematic of an illustrative lentiviral genomic vector with a single transcriptional unit encoding a CAR and an anti-idiotype polypeptide separated by a ribosomal skip sequence.

FIG. 12 shows the expression of 2 different cetuximab anti-idiotype polypeptides on 293T and CHO-S cells as detected by cetuximab and assayed by FACs.

### DEFINITIONS

As used herein, the term “chimeric antigen receptor” or “CAR” or “CARs” refers to engineered receptors, which graft an antigen specificity onto cells, for example T cells, NK cells, macrophages, and stem cells. The CARs of the invention include at least one antigen-specific targeting region (ASTR), a transmembrane domain (TM), and an intracellular activating domain (IAD) and can include a stalk, and one or more co-stimulatory domains (CSDs). In another embodiment, the CAR is a bispecific CAR, which is specific to two different antigens or epitopes. After the ASTR binds specifically to a target antigen, the IAD activates intracellular signaling. For example, the IAD can redirect T cell specificity and reactivity toward a selected target in a non-MHC-restricted manner, exploiting the antigen-binding properties of antibodies. The non-MHC-restricted antigen recognition gives T cells expressing the CAR the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains.

As used herein, the term cell “aggregate” means a cluster of cells that adhere to each other.

As used herein, the term “constitutive T cell or NK cell promoter” refers to a promoter which, when operably linked with a polynucleotide that encodes or specifies a gene product, causes the gene product to be produced in a cell under most or all physiological conditions of the cell.

As used herein, the terms “inducible promoter” or “activatable promoter” refer to promoters that when operably linked with a polynucleotide that encodes or specifies a gene product, cause the gene product to be produced in a cell substantially only when a promoter-specific inducer is present in the cell. Inducible promoters have no, or a low level, of basal transcription activity but the transcription activity increases, sometimes greatly, in the presence of an inducing signal.

As used herein, the term “insulator” refers to a cis-regulatory element that mediates intra- and inter-chromosomal interactions and can block interactions between enhancers and promoters. Typically, insulators are between 200 and 2000 base pairs in length and contain clustered binding sites for sequence specific DNA-binding proteins.

As used herein, the term “microenvironment” means any portion or region of a tissue or body that has constant or temporal, physical, or chemical differences from other regions of the tissue or regions of the body. For example, a “tumor microenvironment” as used herein refers to the environment in which a tumor exists, which is the non-cellular area within the tumor and the area directly outside the tumorous tissue but does not pertain to the intracellular compartment of the cancer cell itself. The tumor microenvironment can refer to any and all conditions of the tumor milieu including conditions that create a structural and or functional environment for the malignant process to survive and/or expand and/or spread. For example, the tumor microenvironment can include alterations in conditions such as, but not limited to, pressure, temperature, pH, ionic strength, osmotic pressure, osmolality, oxidative stress, concentration of one or more solutes, concentration of electrolytes, concentration of glucose, concentration of hyaluronan, concentration of lactic acid or lactate, concentration of albumin, levels of adenosine, levels of R-2-hydroxyglutarate, concentration of pyruvate, concentration of oxygen, and/or presence of oxidants, reductants, or co-factors, as well as other conditions a skilled artisan will understand.

As used interchangeably herein, the terms “polynucleotide” and “nucleic acid” refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, an “approved biologic” is a macromolecule that meets the requirements of a biologic provided by a government regulatory agency such as, but not limited to, the Food And Drug Administration of the U.S. (USFDA), European Medicines Agency (EMA), National Medical Products Administration of China (NMPA) (Chinese FDA), or the Pharmaceutical and Food Safety Bureau (PFSB) of Japan and has been approved by such regulatory agency either as a stand-alone biologic, or as part of a combination product or method.

As used herein, the term “antibody” includes polyclonal and monoclonal antibodies, including intact antibodies and fragments of antibodies which retain specific binding to antigen. The antibody fragments can be, but are not limited to, fragment antigen binding (Fab) fragments, Fab′ fragments, F(ab′)₂ fragments, Fv fragments, Fab′-SH fragments, (Fab′)₂ Fv fragments, Fd fragments, recombinant IgG (rIgG) fragments, single-chain antibody fragments, including single-chain variable fragments (scFv), divalent scFv’s, trivalent scFv’s, and single domain antibody fragments (e.g., sdAb, sdFv, nanobody). The term includes genetically engineered and/or otherwise modified forms of immunoglobulins, such as intrabodies, peptibodies, chimeric antibodies, single-chain antibodies, fully human antibodies, humanized antibodies, fusion proteins including an antigen-specific targeting region of an antibody and a non-antibody protein, heteroconjugate antibodies, multispecific, e.g., bispecific, antibodies, diabodies, triabodies, and tetrabodies, tandem di-scFv’s, and tandem tri-scFv’s. Unless otherwise stated, the term “antibody” should be understood to include functional antibody fragments thereof. The term also includes intact or full-length antibodies, including antibodies of any class or sub-class, including IgG and subclasses thereof, IgM, IgE, IgA, and IgD.

As used herein, the term “antibody fragment” includes a portion of an intact antibody, for example, the antigen binding or variable region of an intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fe” fragment, a designation reflecting the ability to crystallize readily. Pepsin treatment yields an F(ab′)₂ fragment that has two antigen combining sites and is still capable of cross-linking antigen.

As used interchangeably herein, the terms “single-chain Fv,” “scFv,” or “sFv” antibody fragments include the V_{H} and V_{L} domains of antibody, wherein these domains are present in a single polypeptide chain. In some embodiments, the Fv polypeptide further includes a polypeptide linker or spacer between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

As used herein, “naturally occurring” VH and VL domains refer to VH and VL domains that have been isolated from a host without further molecular evolution to change their affinities when generated in an scFv format under specific conditions such as those disclosed in U.S. Pat. 8709755 B2 and application WO/2016/033331A1.

As used herein, “antibody mimetic” refers to an organic compound that specifically binds a target sequence and has a structure distinct from a naturally-occurring antibody. Antibody mimetics may comprise a protein, a nucleic acid, or a small molecule, and a skilled artisan can understand when each type is relevant. The target sequence to which an antibody mimetic of the disclosure specifically binds may be an antigen. Antibody mimetics may provide superior properties over antibodies including, but not limited to, superior solubility, tissue penetration, stability towards heat and enzymes (e.g., resistance to enzymatic degradation), and lower production costs. Antibody mimetics include, but are not limited to, an affibody, an afflilin, an affimer, an affitin, an alphabody, an alphamab, an anticalin, a peptide aptamer, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein),7 a fibrinogen domain, a fibronectin binding domain (FN3 domain) (e.g., adnectin or monobody), a fynomer, a knottin, a Kunitz domain peptide, a nanofitin, a leucine-rich repeat domain, a lipocalin domain, a mAb 2 or Fcab™, a nanobody, a nanoCLAMP, an OBody, a Pronectin, a single-chain TCR, a tetratricopeptide repeat domain, VHH, or a V-like domain.

As used herein, “complementarity-determining region” or “CDR” refers to the three hypervariable regions in each variable chain of immunoglobulins and T cell receptors that interrupt the four “framework” regions of the chains. The CDRs are primarily responsible for the specificity of binding. The CDRs of each immunoglobulin chain are referred to as CDR1, CDR2, and CDR3, numbered sequentially starting from the N-terminus, and are also typically identified by the chain in which the particular CDR is located. Thus, HCDR3 is located in the variable domain of the heavy chain of the antibody in which it is found, whereas a LCDR1 is the CDR1 from the variable domain of the light chain of the antibody in which it is found. The sequences of the framework regions of different light or heavy chains are relatively conserved within a species. The framework region of an antibody, that is the combined framework regions of the constituent light and heavy chains, serves to position and align the CDRs in three dimensional space. The amino acid sequences of the CDRs and framework regions can be determined using various well-known definitions in the art, e.g., Kabat, Chothia, international ImMunoGeneTics database (IMGT), and AbM (see, e.g., Johnson and Wu, Nucleic Acids Res. 2000 Jan 1; 28(1): 214-218 and Johnson et al., Nucleic Acids Res., 29:205-206 (2001); Chothia & Lesk, (1987) J. Mol. Biol. 196, 901-917; Chothia et al. (1989) Nature 342, 877-883; Chothia et al. (1992) J. Mol. Biol. 227, 799-817; Al-Lazikam et al., J. Mol. Biol. 1997, 273(4)). Unless otherwise indicated, CDRs herein are determined using “Fab Analysis” on the World Wide Web at vbase2.org (Retter et al., Nucleic Acids Res., 33:D671-D674 and Mollova et al., BMC Systems Bio., S1, p30)).

As used herein, the term “idiotype” refers to the segment of an antibody or antibody mimetic that determines its specificity for antigen, for example, a structure of a variable region of an antibody, T cell receptor, or antibody mimetic that is shared characteristic between a group of antibodies, T-cell receptors, or antibody mimetics based upon the antigen binding specificity and therefore structure of their variable regions. The idiotype of an antibody typically includes the variable region, e.g., the CDRs and framework regions. For antibodies, the idiotype is located in the Fab region. For antibodies formed with multiple chains, e.g., heavy chains and light chains, expression of the idiotype usually requires participation of the variable regions of both heavy and light chains that form the antigen-combining site. For antibodies formed with single chains, e.g., scFv, expression of the idiotype usually requires participation of the variable regions of one polypeptide that forms the antigen-combining site. For antibody mimetics, the idiotype varies depending on the type of antibody mimetic, but includes the region necessary for binding the cognate antigen.

As used herein, a “therapeutic antibody” or “therapeutic antibody mimetic” is an antibody or antibody mimetic that has been demonstrated using an in vivo assay, for example, in humans, to have therapeutic activity.

As used herein, the term “recognize” refers to the ability of one molecule to bind to another molecule, for example, the ability of a receptor to binds its ligand or the ability of an antibody to bind its target.

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

As used herein, the term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁻⁷ M, e.g., binding with an affinity of 10⁻⁶ M, 10⁻⁵ M, 10⁻⁴ M, etc.

As used herein, reference to a “cell surface expression system” or “cell surface display system” refers to the display or expression of a protein or portion thereof on the surface of a cell. Typically, a cell is generated that expresses proteins of interest fused to a cell-surface protein. For example, a protein is expressed as a fusion protein with a transmembrane domain.

As used herein, the term “element” includes polypeptides, including fusions of polypeptides, regions of polypeptides, and functional mutants or fragments thereof and polynucleotides, including microRNAs and shRNAs, and functional mutants or fragments thereof.

As used herein, the term “region” is any segment of a polypeptide or polynucleotide.

As used herein, a “domain” is a region of a polypeptide or polynucleotide with a functional and/or structural property.

As used herein, the terms “stalk” or “stalk domain” refer to a flexible polypeptide connector region providing structural flexibility and spacing to flanking polypeptide regions and can consist of natural or synthetic polypeptides. A stalk can be derived from a hinge or hinge region of an immunoglobulin (e.g., IgGl) that is generally defined as stretching from Glu216 to Pro230 of human IgG1 (Burton (1985) Molec. Immunol., 22: 161-206). Hinge regions of other IgG isotypes may be aligned with the IgG1 sequence by placing the first and last cysteine residues forming inter-heavy chain disulfide (S-S) bonds in the same positions. The stalk may be of natural occurrence or non-natural occurrence, including but not limited to an altered hinge region, as disclosed in U.S. Pat. No. 5,677,425. The stalk can include a complete hinge region derived from an antibody of any class or subclass. The stalk can also include regions derived from CD8, CD28, or other receptors that provide a similar function in providing flexibility and spacing to flanking regions.

As used herein, the term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

As used herein, a “polypeptide” is a single chain of amino acid residues linked by peptide bonds. A polypeptide does not fold into a fixed structure nor does it have any posttranslational modification. A “protein” is a polypeptide that folds into a fixed structure. “Polypeptides” and “proteins” are used interchangeably herein.

As used herein, a polypeptide may be “purified” to remove contaminant components of a polypeptide’s natural environment, e.g., materials that would interfere with diagnostic or therapeutic uses for the polypeptide such as, for example, enzymes, hormones, and other proteinaceous or nonproteinaceous solutes. A polypeptide can be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain.

As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells).

As used herein, “T cell” includes all types of immune cells expressing CD3 including T-helper cells (CD4⁺ cells), cytotoxic T cells (CD8⁺ cells), T-regulatory cells (Treg) and gamma-delta T cells. NKT cells, which are CD3+, CD56+, and either CD4+ or CD8+, are considered a type of T cells herein. Surface expression of CD3 can be transiently decreased or eliminated in T cells, as has been observed with some of the methods for modifying T cells disclosed herein. Such modified CD4+ or CD8+ lymphocytes that have transiently decreased/absent CD3 surface expression, are still considered T cells in this disclosure. Reference to a “CD” or cluster of differentiation marker, such as CD3+, CD4+, CD8+, CD56+ herein, relates to surface expression of such polypeptide. It will be understood that surface expression is a continuum between positive and negative, and can be assessed by FACS analysis, where cells are determined to be positive or negative based on user cutoffs known in the art. A low and intermediate expression of a surface marker determined by FACS analysis, such as CD3lo or CD3int, are considered surface marker negative (e.g. CD3-) herein.

As used herein, “NK cell” includes lymphocytes that express CD56 on their surface (CD56+ lymphocytes). NKT cells, which are CD3+, CD56+, and either CD4+ or CD8+, are considered a type of NK cells herein.

As used herein, a “cytotoxic cell” includes CD8⁺ T cells, natural-killer (NK) cells, NK-T cells, γδ T cells, a subpopulation of CD4⁺ cells, and neutrophils, which are cells capable of mediating cytotoxicity responses.

As used herein, the term “stem cell” generally includes pluripotent or multipotent stem cells. “Stem cells” includes, e.g., embryonic stem cells (ES); mesenchymal stem cells (MSC); induced-pluripotent stem cells (iPS); and committed progenitor cells (hematopoietic stem cells (HSC); bone marrow derived cells, etc.).

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As used interchangeably herein, the terms “individual”, “subject”, “host”, and “patient” refer to a mammal, including, but not limited to, humans, murines (e.g., rats, mice), lagomorphs (e.g., rabbits), non-human primates, humans, canines, felines, ungulates (e.g., equines, bovines, ovines, porcines, caprines), etc.

As used herein, the terms “therapeutically effective amount” or “efficacious amount” refers to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

As used herein, the term “evolution” or “evolving” refers to using one or more methods of mutagenesis to generate a different polynucleotide encoding a different polypeptide, which is itself an improved biological molecule and/or contributes to the generation of another improved biological molecule. “Physiological” or “normal” or “normal physiological” conditions are conditions such as, but not limited to, pressure, temperature, pH, ionic strength, osmotic pressure, osmolality, oxidative stress, concentration of one or more solutes, concentration of electrolytes, concentration of glucose, concentration of hyaluronan, concentration of lactic acid or lactate, concentration of albumin, levels of adenosine, levels of R-2-hydroxyglutarate, concentration of pyruvate, concentration of oxygen, and/or presence of oxidants, reductants, or co-factors, as well as other conditions, that would be considered within a normal range at the site of administration, or at the tissue or organ at the site of action, to a subject.

As used herein, a “transduced cell” or a “stably transfected cell” is a cell that contains an exogenous nucleic acid(s) that is integrated into the genome of the cell. As used herein, a “genetically modified cell” is a cell that contains an exogenous nucleic acid(s) regardless of whether the exogenous nucleic acid(s) is integrated into the genome of the cell, and regardless of the method used to introduce the exogenous nucleic acid(s) into the cell. Exogenous nucleic acid(s) inside a cell that are not integrated into the genome of the cell can be referred to as “extrachromosomal” herein. As used herein, a “modified cell” is a cell that is associated with a recombinant nucleic acid vector (also called a “polynucleotide vector” or a “gene vector” herein), which in illustrative embodiments is a replication incompetent recombinant retroviral particle (also called a “RIR retroviral particle” or a “RIP” herein), that contains an exogenous nucleic acid, or a cell that has been genetically modified by an exogenous nucleic acid. Typically, in compositions and methods that include a replication incompetent recombinant retroviral particle, a modified cell associates with a replication incompetent recombinant retroviral particle through interactions between proteins on the surface of the cell and proteins on the surface of the replication incompetent recombinant retroviral particle, including pseudotyping elements and/or T cell activation elements. In compositions and methods that include transfection of nucleic acid inside a lipid-based reagent, such as a liposomal reagent, the lipid-based reagent containing nucleic acid, which is a type of recombinant nucleic acid vector, associates with the lipid bilayer of the modified cell before fusing or being internalized by the modified cell. Similarly, in compositions and methods that include chemical-based transfection of nucleic acid, such as polyethylenimine (PEI) or calcium phosphate-based transfection, the nucleic acid is typically associated with a positively charged transfection reagent to form the recombinant nucleic acid vector that associates with the negatively charged membrane of the modified cell before the complex is internalized by the modified cell. Other means or methods of stably transfecting or genetically modifying cells include electroporation, ballistic delivery, and microinjection. A “polypeptide” as used herein can include part of or an entire protein molecule as well as any posttranslational or other modifications.

A pseudotyping element as used herein can include a “binding polypeptide” that includes one or more polypeptides, typically glycoproteins, that identify and bind the target host cell, and one or more “fusogenic polypeptides” that mediate fusion of the retroviral and target host cell membranes, thereby allowing a retroviral genome to enter the target host cell. The “binding polypeptide” as used herein, can also be referred to as a “T cell and/or NK cell binding polypeptide” or a “target engagement element,” and the “fusogenic polypeptide” can also be referred to as a “fusogenic element”.

A “resting” lymphocyte, such as for example, a resting T cell, is a lymphocyte in the G0 stage of the cell cycle that does not express activation markers such as Ki-67. Resting lymphocytes can include naïve T cells that have never encountered specific antigen and memory T cells that have been altered by a previous encounter with an antigen. A “resting” lymphocyte can also be referred to as a “quiescent” lymphocyte.

As used herein, “lymphodepletion” involves methods that reduce the number of lymphocytes in a subject, for example by administration of a lymphodepletion agent. Lymphodepletion can also be attained by partial body or whole-body fractioned radiation therapy. A lymphodepletion agent can be a chemical compound or composition capable of decreasing the number of functional lymphocytes in a mammal when administered to the mammal. One example of such an agent is one or more chemotherapeutic agents. Such agents and dosages are known, and can be selected by a treating physician depending on the subject to be treated. Examples of lymphodepletion agents include, but are not limited to, fludarabine, cyclophosphamide, cladribine, denileukin diftitox, alemtuzumab or combinations thereof.

RNA interference (RNAi) is a biological process in which RNA molecules inhibit gene expression or translation by neutralizing targeted RNA molecules. The RNA target may be mRNA, or it may be any other RNA susceptible to functional inhibition by RNAi. As used herein, an “inhibitory RNA molecule” refers to an RNA molecule whose presence within a cell results in RNAi and leads to reduced expression of a transcript to which the inhibitory RNA molecule is targeted. An inhibitory RNA molecule as used herein has a 5′ stem and a 3′ stem that is capable of forming an RNA duplex. The inhibitory RNA molecule can be, for example, a miRNA (either endogenous or artificial) or a shRNA, a precursor of a miRNA (i.e., a Pri-miRNA or Pre-miRNA) or shRNA, or a dsRNA that is either transcribed or introduced directly as an isolated nucleic acid, to a cell or subject.

As used herein, “double stranded RNA” or “dsRNA” or “RNA duplex” refers to RNA molecules that are comprised of two strands. Double-stranded molecules include those comprised of two RNA strands that hybridize to form the duplex RNA structure or a single RNA strand that doubles back on itself to form a duplex structure. Most, but not necessarily all of the bases in the duplex regions are base-paired. The duplex region comprises a sequence complementary to a target RNA. The sequence complementary to a target RNA is an antisense sequence, and is frequently from 18 to 29, from 19 to 29, from 19 to 21, or from 25 to 28 nucleotides long, or in some embodiments between 18, 19, 20, 21, 22, 23, 24, 25 on the low end and 21, 22, 23, 24, 25, 26, 27, 28 29, or 30 on the high end, where a given range always has a low end lower than a high end. Such structures typically include a 5′ stem, a loop, and a 3′ stem connected by a loop which is contiguous with each stem and which is not part of the duplex. The loop comprises, in certain embodiments, at least 3, 4, 5, 6, 7, 8, 9, or 10 nucleotides. In other embodiments the loop comprises from 2 to 40, from 3 to 40, from 3 to 21, or from 19 to 21 nucleotides, or in some embodiments between 2, 3, 4, 5, 6, 7, 8, 9, 10, 11, 12, 13, 14, 15, 16, 17, 18, 19, or 20 on the low end and 10, 11, 12, 13, 14, 15, 16, 17, 18, 19, 20, 25, 30, 35, or 40 on the high end, where a given range always has a low end lower than a high end.

The term “microRNA flanking sequence” as used herein refers to nucleotide sequences including microRNA processing elements. MicroRNA processing elements are the minimal nucleic acid sequences which contribute to the production of mature microRNA from precursor microRNA. Often these elements are located within a 40-nucleotide sequence that flanks a microRNA stem-loop structure. In some instances, the microRNA processing elements are found within a stretch of nucleotide sequences of between 5 and 4,000 nucleotides in length that flank a microRNA stem-loop structure.

The term “linker” when used in reference to a multiplex inhibitory RNA molecule refers to a connecting means that joins two inhibitory RNA molecules.

As used herein, a “recombinant retrovirus” refers to a non-replicable, or “replication incompetent”, retrovirus unless it is explicitly noted as a replicable retrovirus. The terms “recombinant retrovirus” and “recombinant retroviral particle” are used interchangeably herein. Such retrovirus/retroviral particle can be any type of retroviral particle including, for example, gamma retrovirus, and in illustrative embodiments, lentivirus. As is known, such retroviral particles, for example lentiviral particles, typically are formed in packaging cells by transfecting the packing cells with plasmids that include packaging components such as Gag, Pol and Rev, an envelope or pseudotyping plasmid that encodes a pseudotyping element, and a transfer, genomic, or retroviral (e.g., lentiviral) expression vector, which is typically a plasmid on which a gene(s) or other coding sequence of interest is encoded. Accordingly, a retroviral (e.g., lentiviral) expression vector includes sequences (e.g., a 5′ LTR and a 3′ LTR flanking e.g., a psi packaging element and a target heterologous coding sequence) that promote expression and packaging after transfection into a cell. The terms “lentivirus” and “lentiviral particle” are used interchangeably herein.

A “framework” of a miRNA consists of “5′ microRNA flanking sequence” and/or “3′ microRNA flanking sequence” surrounding a miRNA and, in some cases, a loop sequence that separates the stems of a stem-loop structure in a miRNA. In some examples, the “framework” is derived from naturally occurring miRNAs, such as, for example, miR-155. The terms “5′ microRNA flanking sequence” and “5′ arm” are used interchangeably herein. The terms “3′ microRNA flanking sequence” and “3′ arm” are used interchangeably herein.

As used herein, the term “miRNA precursor” refers to an RNA molecule of any length which can be enzymatically processed into an miRNA, such as a primary RNA transcript, a pri-miRNA, or a pre-miRNA.

As used herein, the term “construct” refers to an isolated polypeptide or an isolated polynucleotide encoding a polypeptide. A polynucleotide construct can encode a polypeptide, for example, a lymphoproliferative element. A skilled artisan will understand whether a construct refers to an isolated polynucleotide or an isolated polypeptide depending on the context.

As used herein, “MOI”, refers to Multiplicity of Infection ratio where the MOI is equal to the ratio of the number of virus particles used for infection per number of cells. Functional titering of the number of virus particles can be performed using FACS and reporter expression, as non-limiting examples.

“Peripheral blood mononuclear cells” (PBMCs) include peripheral blood cells having a round nucleus and include lymphocytes (e.g., T cells, NK cells, and B cells) and monocytes. Some blood cell types that are not PBMCs include red blood cells, platelets and granulocytes (i.e., neutrophils, eosinophils, and basophils).

It is to be understood that the present disclosure and the aspects and embodiments provided herein, are not limited to particular examples disclosed, as such may, of course, vary. It is also to be understood that the terminology used herein is for the purpose of disclosing particular examples and embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. When multiple low and multiple high values for ranges are given that overlap, a skilled artisan will recognize that a selected range will include a low value that is less than the high value. All headings in this specification are for the convenience of the reader and are not limiting.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a chimeric antigen receptor” includes a plurality of such chimeric antigen receptors and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless specifically stated or otherwise obvious from context, as used herein, the term “or” is understood to be inclusive. The term “and/or” as used in a phrase such as “A and/or B” herein includes each of the following: A and B; A or B; A (alone); and B (alone). Similarly, the term “and/or” as used in a phrase such as “A, B, and/or C” includes each of the following: A, B, and C; A, B, or C; A or B; A or C; B or C; A and B; A and C; B and C; A (alone); B (alone); and C (alone). This logic extends to any number of items in a list that are connected with the term “and/or”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### DETAILED DESCRIPTION

The present disclosure overcomes prior art challenges by providing improved methods and compositions for selectively killing or modulating the activity of modified cells, for example, modified NK cells and in illustrative embodiments, modified T cells, using anti-idiotype polypeptides expressed on the surface of the cells. For example, provided herein are anti-idiotype polypeptides that are improved cell therapy safety switches. One challenge with prior art safety switches that are eTags, is that they include EGFR family member domains, and thus are limited in structure to only EGFR domains. Furthermore, they may yield undesirable side effects when delivered to a subject since they are structurally related to a growth factor (e.g. EGFR) found in a subject. The anti-idiotype polypeptide safety switches provided herein, are much more robust and flexible safety switches than eTags in terms of design and development. Anti-idiotype polypeptide safety switch can be designed to be recognized by virtually any antibody, including any clinical antibody, such as a therapeutic antibody. Exemplary methods for developing anti-idiotype antibodies, which utilize phage display assays, are disclosed in the Examples section herein. Furthermore, the anti-idiotype polypeptides provided herein include designs that can be used to modulate cell activity, including, but not limited to, proliferation and apoptosis, and that do not require antibodies that drive cytotoxicity.

More specifically, some of the methods and compositions herein, provide versatile and effective solutions for controlling cytokine release syndrome in a subject with modified cells expressing a chimeric antigen receptor (CAR) or recombinant T cell receptor (TCR). Thus, the methods provide an important step toward improving the safety and effectiveness of cell therapy methods. Illustrative compositions for selectively killing of modified cells, for example, modified NK cells and in illustrative embodiments, modified T cells, are easier to develop and safer than existing compositions and methods. Some aspects of the anti-idiotype polypeptides provided herein, methods using the same, and polynucleotides encoding the same provide new methods and compositions to modulate the activity of the modified cells. Furthermore, compositions that have many uses, including their use in these improved methods, are provided, including cell formulation compositions. Some of these compositions include modified and in illustrative embodiments genetically modified lymphocytes that include safety switches for use with cellular therapies to affect the reduction or elimination of infused cells if dangerous adverse events develop. Other of these compositions have improved proliferative and survival qualities, including in in vitro culturing, for example in the absence of growth factors. Such modified and in illustrative embodiments genetically modified lymphocytes will have utility for example, as research tools to better understand mechanisms to control and stop cytokine release syndrome and the factors that influence T cell proliferation and survival, and for commercial production, for example for the production of certain factors, such as growth factors and immunomodulatory agents, that can be harvested and tested or used in commercial products. And such modified and genetically modified lymphocytes have utility in the treatment of cancer and other diseases.

### Anti-Idiotype Polypeptides

Provided herein are anti-idiotype polypeptides, and polynucleotides encoding these polypeptides (as disclosed in detail in other sections herein), that have numerous utilities in life sciences and medicine. Such polypeptides in illustrative embodiments are especially useful in modified cells, for example for use in cell and gene therapy. Anti-idiotype polypeptides expressed on the surface of cells can recognize target antibodies or target antibody mimetics that come in contact with these cells. These antibodies and antibody mimetics can be used to, for example mark cells expressing the anti-idiotype polypeptides for killing by the immune system, modulate a property (such as, for example, a proliferative state or an apoptotic state) or activity of the cells, label the cells, provide a target for enrichment and/or purification, enrich the cells, or cause the cells to aggregate. A person skilled in the art will understand how to use the anti-idiotype polypeptides for these and other methods in view of the present disclosure. Accordingly, provided herein are methods for providing any of the above-mentioned uses, by expressing any of the polynucleotides that are disclosed herein, that include nucleic acids encoding anti-idiotype polypeptides disclosed herein.

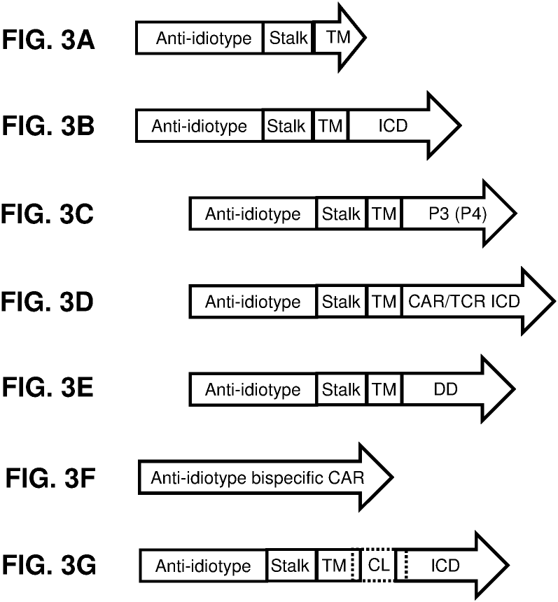

FIGS. 3 shows non-limiting exemplary embodiments of anti-idiotype polypeptides herein. As shown in FIG. 3A, anti-idiotype polypeptides herein, in illustrative aspects include an extracellular recognition domain (sometimes referred to as an anti-idiotype extracellular recognition domain, anti-id ERD, or anti-Id ECD and denoted in FIGS. 3A-F and FIG. 3H as “anti-idiotype”) and typically include a membrane association domain (MAD), which is separated from the anti-id ECD in illustrative embodiments, by a stalk. The anti-Id ERD in illustrative embodiments includes a recognition domain of an anti-idiotype antibody or anti-idiotype antibody mimetic. The recognition domain of an anti-idiotype polypeptide recognizes the idiotype of a target antibody or the idiotype of a target antibody mimetic. In illustrative embodiments, an anti-idiotype extracellular recognition domain includes an idiotype-binding variable region of an anti-idiotype antibody or anti-idiotype antibody mimetic. In illustrative embodiments, a stalk separates the MAD and the anti-idiotype extracellular recognition domain.

In some embodiments, an extracellular recognition domain recognizes the idiotype of any antibody or antibody mimetic known in the art. In certain illustrative embodiments, the extracellular recognition domain recognizes the idiotype of a clinical antibody or clinical antibody mimetic. Such a clinical antibody, in some illustrative embodiments, is a regulatory agency (e.g., U.S. FDA) approved biologic. In some embodiments, binding of the anti-idiotype polypeptide to the target antibody does not block or prevent binding between the target antibody and its cognate antigen. In illustrative embodiments, binding of the anti-idiotype polypeptide to the target antibody blocks or prevents binding between the target antibody and its cognate antigen.

Typically, anti-idiotype polypeptides include a membrane association domain (sometimes referred to herein as MAD). The membrane association domain of the anti-idiotype polypeptide attaches, tethers, or anchors the recognition domain from an anti-idiotype antibody or antibody mimetic to a cell membrane. In some embodiments, the membrane association domain comprises one or more of a transmembrane domain, and a GPI anchor, as further disclosed elsewhere herein. In some embodiments, the transmembrane domain can be a heterologous transmembrane domain, or an endogenous transmembrane domain, either of which could be the transmembrane domain of an antibody.

As shown in FIGS. 3B-G, in some embodiments, anti-idiotype polypeptides provided herein further include one or more intracellular domains (sometimes referred to herein as ICD). Furthermore, the MAD of such anti-idiotype polypeptides that include ICDs in certain illustrative embodiments, is a transmembrane domain (“TM” in FIGS. 3B-G). The one or more intracellular domains can activate or inhibit pro-apoptotic or anti-apoptotic pathways and/or pro-survival or anti-survival pathways, and in certain embodiments modulate other cellular processes/pathways. Details are provided throughout this specification regarding these various embodiments and other embodiments wherein an anti-idiotype polypeptide herein, includes an ICD. FIGS. 3B-G provides some non-limiting examples. In some embodiments, the ICD serves a structural role to assure the anti-id ERD is stably expressed on and remains bound to the cell membrane. In other embodiment an ICD can have functional properties that are regulated by binding dimerization or multimerization that is induced by binding of the anti-id ERD by its target antibody or antibody mimetic.

In some embodiments (FIG. 3C), the anti-id ERD (“anti-idiotype” in FIG. 3C) acts as an inducible extracellular dimerizing domain of an LE herein. In such embodiments, dimerization of an LE by binding of a target antibody to the anti-id ERD can activate signaling domains in the ICD, driving proliferation and/or cell survival. As illustrated, the ICD of an LE can include P3 and optionally P4 domains, as disclosed herein with respect to LE ICDs. In some embodiments, the intracellular domains can activate one or more of a Jak/Stat pathway, a TRAF pathway, a PI3K pathway, or a PLC pathway. Disclosure related to mechanisms for activating these pathways is provided in the “Lymphoproliferative section” herein. Illustrative examples of these embodiments are inducible chimeric lymphoproliferative elements, which are inducible upon binding of a target antibody to an extracellular domain that recognizes the idiotype of a target antibody.

In some embodiments (FIG. 3D), the anti-id ERD (“anti-idiotype” in FIG. 3D) acts as the ASTR of a CAR or TCR. Thus, the CAR or TCR includes the anti-id ERD, a stalk attaching the ERD to a transmembrane domain, and an ICD of a CAR or TCR. Thus, binding of a target antibody to the anti-id ERD can activate signaling domains in the ICD of the CAR or TCR.

In some embodiments, the intracellular domain is pro-apoptotic, and can include one or more intracellular signaling domains from a caspase protein and/or one or more intracellular signaling domains from tumor necrosis factor receptor superfamily members. As such, such embodiments are specific examples of safety switches provided herein. As illustrated in FIG. 3E, such embodiments can include an anti-idiotype polypeptide wherein the ICD includes a death domain. Such ICDs can include all or in certain illustrative embodiments, include a portion of an ICD from a TNF receptor superfamily member, that includes a death domain, such as FAS. Illustrative embodiments of such an ant-idiotype polypeptide includes a constitutive dimerization domain in the extracellular domain, that can be all or part of the stalk or transmembrane domain, thus providing such anti-idiotype polypeptide, the ability to form higher order multimers, such as tetramers.. Furthermore, ICDs for any embodiment illustrated in FIG. 3E can include death domains, or other functional domains from initiator caspases, such as for example, caspases 2, 8, 9, and 10.

In some embodiments (FIG. 3F), the anti-id ERD (“anti-idiotype” in FIG. 3D) acts as one of the ASTRs of a bi-specific CAR or TCR, wherein a second ASTR is present that typically binds to an antigen expressed by a cancer cell. Such embodiments can be called anti-idiotype bispecific CARs herein. In such embodiments, the CAR or TCR includes the anti-id ERD, a second ASTR, a stalk attaching the anti-id ERD and the 2^{nd} ASTR to a transmembrane domain, and an ICD of a CAR or TCR. Accordingly, binding of a target antibody to the anti-id ERD or binding of the 2^{nd} ASTR to its antigen, can activate signaling domains in the ICD of the CAR or TCR.

FIG. 3G illustrates another embodiment of anti-idiotype polypeptides herein that includes an ICD. In such embodiment, a cleavage site, typically that is activated by dimerization, is added to the anti-idiotype polypeptide between or as part of the transmembrane domain and the ICD. As such, in some embodiments these anti-idiotype polypeptides include a transmembrane domain that includes an amino acid sequence that serves as a substrate cleavage site upon dimerization, by certain proteases. Such cleavage site can be, in certain embodiments, a substrate cleavage site for gamma-secretase complex, as discussed in more detail herein. The intracellular domain for such embodiments can encode various intracellular polypeptides including certain caspases, including initiator caspases, for example caspases 2, 8, 9, and 10. In other embodiments, the ICD is a transcription factor. In such embodiments, the transcription factor is sequestered outside the nucleus until binding of the anti-id ERD by its target antibody inducing dimerization and cleavage, which releases the transcription factor such that it can enter the nucleus to become active in regulating gene expression.

### Anti-Idiotype Polynucleotides

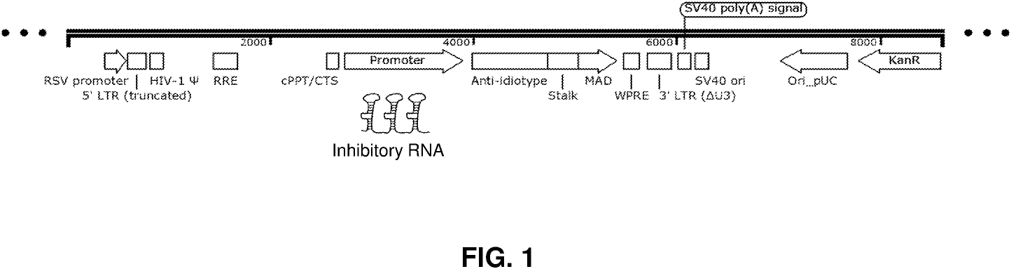

Provided herein in one aspect, are polynucleotides that include nucleic acids that encode anti-idiotype polypeptides, which can be referred to herein as anti-idiotype polynucleotides. FIG. 1 provides a non-limiting exemplary polynucleotide that is a portion of a lentiviral vector, in this non-limiting example a third-generation lentiviral expression vector, that includes nucleic acids that encode an anti-idiotype polypeptide. The encoded anti-idiotype polypeptide includes an anti-idiotype extracellular recognition domain (“Anti-idiotype”), a stalk, and a membrane association domain (MAD). The non-limiting polynucleotide vector of FIG. 1 includes a promoter (“Promoter”) positioned to drive expression of nucleic acids encoding different functional polypeptides including the anti-idiotype polypeptide. Furthermore, the polynucleotide vector of FIG. 1 includes various functional domains as indicated, including various viral functional domains, and encodes an optional inhibitory RNA molecule within the promoter.

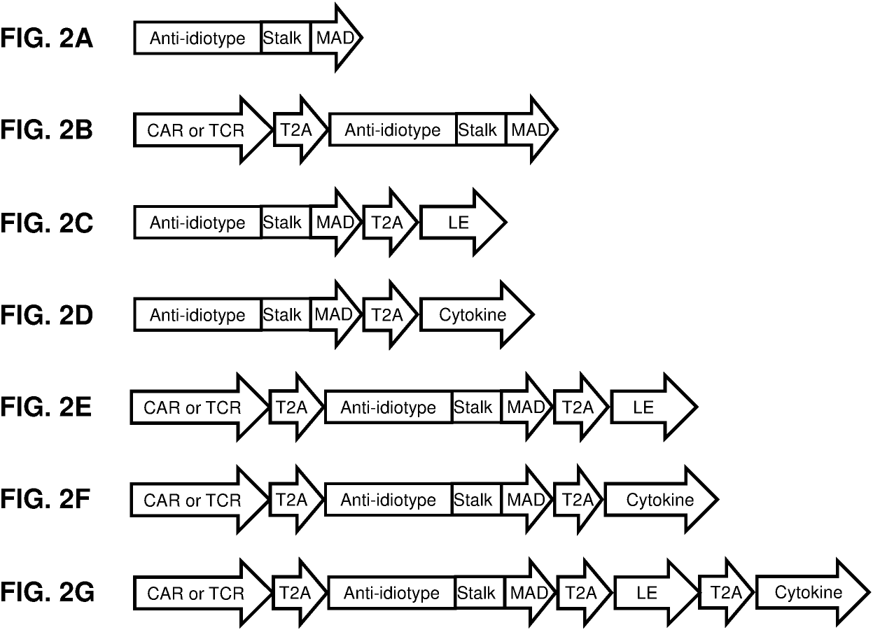

FIGS. 2 provides non-limiting examples of some polynucleotides provided herein, that include nucleic acids that encode one or more anti-idiotype polypeptides, which themselves represent separate aspects herein. As shown in FIG. 2A, nucleic acids that encode an anti-idiotype polypeptide, in some embodiments,encode a membrane association domain (MAD) and an anti-idiotype extracellular recognition domain (referred to as “anti-idiotype” in FIGS. 2). In illustrative embodiments, nucleic acids encoding a stalk separate and are in frame with nucleic acids encoding the MAD and nucleic acids encoding the anti-idiotype extracellular recognition domain. In some embodiments, provided herein are polynucleotides that encode additional functionalities, expressed from the same promoter (i.e., on the same transcription unit) or from different promoters (i.e. different transcriptional units). For example, polynucleotides that include nucleic acids that encode an anti-idiotype extracellular recognition domain, can include nucleic acids that encode, in addition to an anti-idiotype extracellular recognition domain, an engineered signaling polypeptide, a cytokine, and/or an inhibitory RNA. Thus, provided herein in one aspect is a polynucleotide, comprising: one or more transcriptional units, wherein each of the one or more transcriptional units is operatively linked to a promoter, wherein the one or more transcriptional units comprise:
a) a polynucleotide sequence encoding one or more inhibitory RNA molecules, a first engineered signaling polypeptide, and/or a cytokine, and
b) a polynucleotide sequence encoding an anti-idiotype polypeptide comprising an anti-idiotype extracellular recognition domain that recognizes an idiotype of a target antibody or a target antibody mimetic.

Polynucleotides that include nucleic acids that encode anti-idiotype polypeptides can be DNA or RNA. In some illustrative embodiments, they are mRNA. Such embodiments can include embodiments wherein the anti-idiotype antibody is directed against the antibody that forms the ASTR of a CAR. As such, mRNA that encode an anti-idiotype antibody can be directly delivered to a subject and when taken up and expressed by cells in the subject, such cells can form artificial antigen presenting cells that drive proliferation of CAR-T cells administered to the subject that express a CAR with an ASTR that is the target antibody recognized by the anti-idiotype polypeptide.

Methods for making synthetic mRNA are well known in the art. Furthermore, such polynucleotides can be polynucleotide vectors, such as expression vectors. Further details regarding polynucleotides and polynucleotide vectors, such as RIPs, including lentiviral particles, are provided throughout this disclosure, including the claims. These further details include details regarding polynucleotides that include nucleic acids encoding anti-idiotype polypeptides. Some non-limiting noteworthy sections include the Recombinant Retroviral Particles section, the Nucleic Acids section, and the Exemplary Embodiments section.

Nucleic acids encoding the anti-idiotype polypeptide can be upstream or downstream (i.e. 5′or 3′) from those encoding other functionalities. Thus, in such embodiments, anti-idiotype polynucleotides are expressed as a separate polypeptide from other functional polypeptides.

In certain illustrative embodiments, polynucleotides herein encode an anti-idiotype polypeptide and are adapted for, structured for, and/or effective for expression in T cells and/or NK cells, and thus for T cell and/or NK cell therapies. Examples of such polynucleotides typically include a promoter that is active in T cells and/or NK cells, that drives expression of the anti-idiotype extracellular recognition domain and a membrane association domain, which thus are on the same transcriptional unit whose expression is driven by the promoter. Accordingly, in some embodiments, an anti-idiotype polypeptide is expressed as part of a single polynucleotide that also encodes a chimeric antigen receptor (CAR), an engineered T cell receptor (TCR) (FIG. 2B), a lymphoproliferative element (FIG. 2C), or a cytokine (FIG. 2D). Furthermore, in some embodiments, an anti-idiotype polypeptide is expressed as part of a single polynucleotide that encodes a CAR or a TCR, an anti-idiotype polypeptide, and a lymphoproliferative element (FIG. 2E), or a cytokine (FIG. 2F), or both a lymphoproliferative element and a cytokine (FIG. 2G). Such polynucleotide embodiments and the anti-idiotype polypeptides they encode, especially for embodiments where both an anti-idiotype polypeptide and a CAR or TCR are encoded, when present and expressed in T cells and/or NK cells, are especially suited for, adapted for, and/or effective as safety switches as provided herein, including as part of CAR-T therapy, TIL therapy, and CAR-NK therapy, and other safety switch methods provided herein.

In some embodiments the polynucleotide encoding the anti-idiotype polypeptide is separated from the polynucleotide encoding the CAR, the TCR, the cytokine, and/or the polynucleotide encoding the lymphoproliferative element by an internal ribosome entry site (IRES) or a ribosomal skip sequence and/or cleavage signal (as shown, for example, in FIG. 2B-FIG. 2G). The IRES or ribosomal skip and/or cleavage signal can be any IRES or ribosomal skip sequence and/or cleavage signal known in the art. The ribosomal skip sequence can be, for example, T2A with amino acid sequence GSGEGRGSLLTCGDVEENPGP (SEQ ID NO:83). Other examples of cleavage signalsand/or ribosomal skip sequences include FMDV 2A (F2A); equine rhinitis A virus 2A (abbreviated as E2A); porcine teschovirus-1 2A (P2A); and Thoseaasigna virus 2A (T2A).

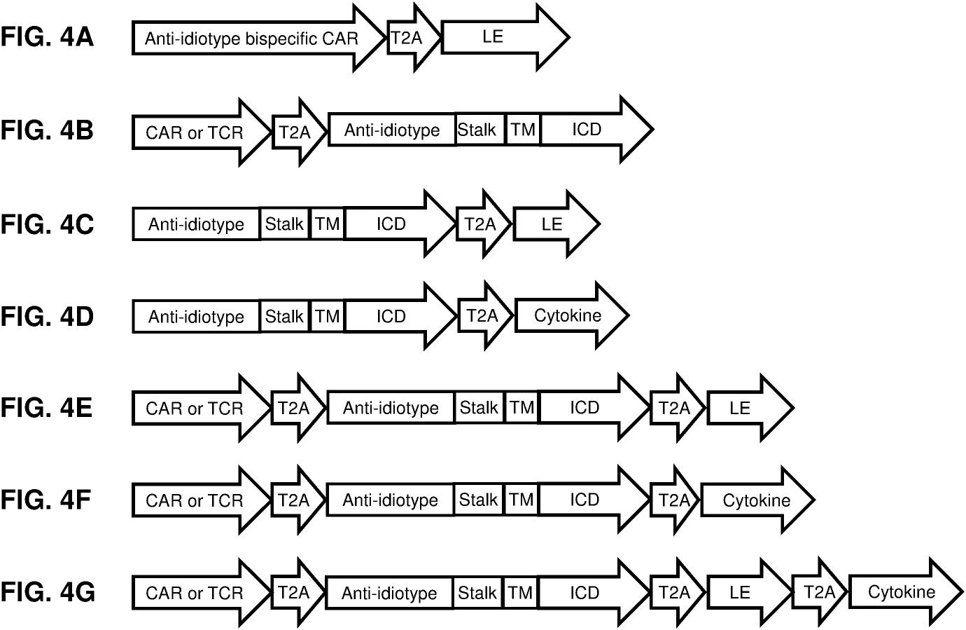

FIGS. 4 provides additional examples of anti-idiotype polynucleotides provided herein. For example, FIG. 4A illustrates a polynucleotide construct that includes nucleic acids encoding a bispecific CAR (i.e. a CAR with an anti-id ERD as one ASTR), followed by a nucleic acid T2A element, followed by nucleic acids encoding an LE. FIG. 4B illustrates a polynucleotide construct that includes nucleic acids encoding a CAR or TCR, followed by a nucleic acid T2A element, followed by nucleic acids encoding an anti-idiotype polypeptide that includes an anti-id ERD, a stalk, a transmembrane domain, and an intracellular domain. FIG. 4C illustrates a polynucleotide construct that includes nucleic acids encoding an anti-idiotype polypeptide that includes an anti-id ERD, a stalk, a transmembrane domain, and an intracellular domain, followed by a nucleic acid T2A element, followed by nucleic acids encoding an LE. FIG. 4D illustrates a polynucleotide construct that includes nucleic acids encoding an anti-idiotype polypeptide that includes an anti-id ERD, a stalk, a transmembrane domain, and an intracellular domain, followed by a nucleic acid T2A element, followed by nucleic acids encoding a cytokine. FIG. 4E illustrates a polynucleotide construct identical to FIG. 4B, but where the nucleic acids encoding the ICD of the anti-idiotype polypeptide are followed by a nucleic acid T2A element, which is followed by nucleic acids encoding an LE. FIG. 4F illustrates a polynucleotide construct identical to FIG. 4B, but where the nucleic acids encoding the LE are followed by a nucleic acid T2A element, which is followed by nucleic acids encoding a cytokine. FIG. 4G illustrates a polynucleotide construct identical to FIG. 4E, but where the nucleic acids encoding the ICD of the anti-idiotype polypeptide are followed by a nucleic acid T2A element, which is followed by nucleic acids encoding a cytokine.

Some non-limiting specific exemplary constructs, are the following:

F1-3-247 encoding a CD19 CAR and a polypeptide lymphoproliferative element comprised from amino to carboxy terminus of the Kozak-type sequence GCCGCCACCAT/UG(G) (SEQ ID NO:331), having the T at the “T/U” residue and having the optional last G, the CD8 signal peptide MALPVTALLLPLALLLHAARP (SEQ ID NO:72) (in which the sequence ATGG from the Kozak-type sequence also encodes the first four nucleotides of the CD8 signal peptide), a FLAG-TAG (DYKDDDDK; SEQ ID NO:74), a linker (GSTSGS; SEQ ID NO:349), an anti-CD19scFv, a CD8 stalk and transmembrane region, and an intracellular domain from CD3z followed by T2A and the lymphoproliferative element comprising the parts E006-T016-S186-S050 which encode an extracellular domain containing a variant of c-Jun including a leucine zipper motif and an eTag, the transmembrane domain of CSF2RA, the intracellular domain of MPL, and the intracellular domain of CD40 with each part of the lymphoproliferative element connected by a GGS linker.

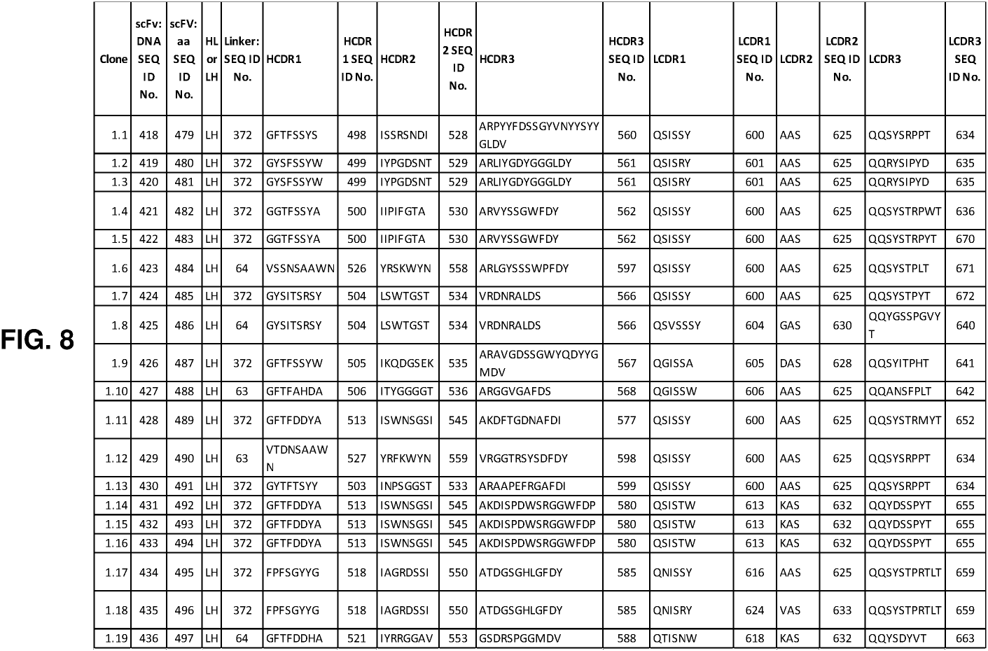

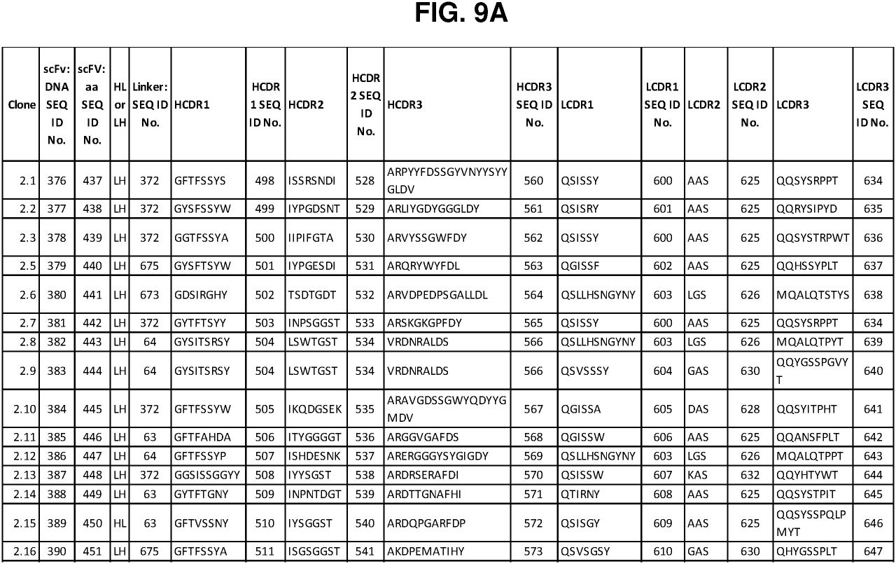

F1-3-P100 encoding a CD19 CAR and an anti-idiotype polypeptide with the P3 and P4 domains of an LE comprised, from amino to carboxy terminus, of the Kozak-type sequence GCCGCCACCAT/UG(G) (SEQ ID NO:331), having the T at the “T/U” residue and having the optional last G, the CD8 signal peptide MALPVTALLLPLALLLHAARP (SEQ ID NO:72) (in which the sequence ATGG from the Kozak-type sequence also encodes the first four nucleotides of the CD8 signal peptide), a FLAG-TAG (DYKDDDDK; SEQ ID NO:74), a linker (GSTSGS; SEQ ID NO:349), an anti-CD19scFv, a CD8 stalk and transmembrane region, and an intracellular domain from CD3z followed by T2A an anti-cetuximab scFv from the table shown in FIG. 8, a stalk, transmembrane and ICD (STMICD2, SEQ ID NO. 677) and the lymphoproliferative element parts S121-S105 which encode the intracellular domain of IL7Rα, and the intracellular domain of IL2Rβ connected by a GGS linker.

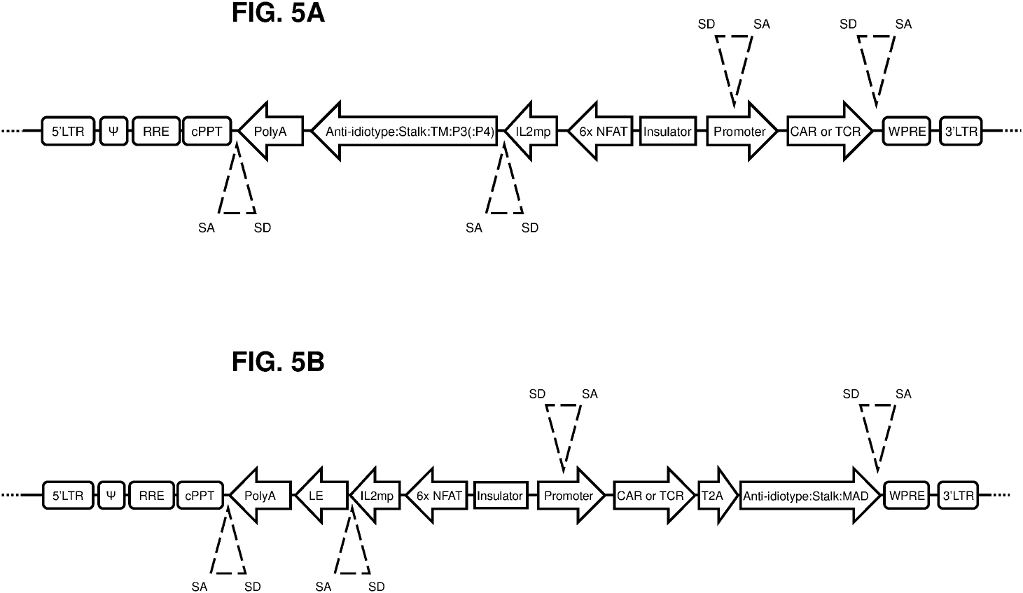

FIG. 5A shows a schematic of an illustrative bicistronic lentiviral genomic vector with divergent transcriptional units. A first transcriptional unit comprising a lymphoproliferative element that is also anti-idiotype polynucleotide, followed by a polyadenylation sequence (PolyA) under the transcriptional control of an NFAT-responsive minimal IL-2 promoter (6x NFAT) is encoded in the reverse orientation. Optionally, an insulator element (Ins) separates the first and second transcriptional units. The second transcriptional unit encodes a CAR (CAR) under the transcriptional control of a constitutive promoter (Promoter) and is encoded in the forward orientation. Triangles shown in dashed lines represent 3 possible locations into any one or more of which, one or more miRNAs could optionally be inserted into the vector. The triangle shown in a dotted line represents 1 possible location in an exon within a promoter such as for EF1-α into which one or more inhibitory RNAs could optionally be inserted into the vector. “SA” and “SD” correspond to splice donor and splice acceptor sites.

FIG. 5B shows a schematic of an illustrative bicistronic lentiviral genomic vector with divergent transcriptional units. A first transcriptional encoding a lymphoproliferative element followed by a polyadenylation sequence (PolyA) under the transcriptional control of an NFAT-responsive minimal IL-2 promoter (6x NFAT) is encoded in the reverse orientation. Optionally, an insulator element (Ins) separates the first and second transcriptional units. The second transcriptional unit encodes a CAR (CAR) followed by a T2A element and an anti-idiotype extracellular recognition domain, a stalk, and a MAD under the transcriptional control of a constitutive promoter (Promoter) and is encoded in the forward orientation. Triangles shown in dashed lines represent 3 possible locations into any one or more of which, one or more miRNAs could optionally be inserted into the vector. The triangle shown in a dotted line represents 1 possible location in an exon within a promoter such as for EF1-α into which one or more inhibitory RNAs could optionally be inserted into the vector. “SA” and “SD” correspond to splice donor and splice acceptor sites. The ECD of the lymphoproliferative element in this design optionally comprises an eTAG or second anti-idiotype extracellular recognition domain.F1-6-744 encodes a bicistronic lentiviral genomic vector with divergent transcriptional units. The first transcriptional unit encodes the lymphoproliferative element E016-T016-S186-S050 under the control of an NFAT-responsive minimal IL-2 promoter all encoded in the reverse orientation. The second transcriptional unit encodes a HER2 CAR comprised of an anti-HER2scFv, a CD8 stalk and transmembrane region, a CD137 intracellular domain, and an intracellular activating domain from CD3z followed by T2A and an eTag. The first and second transcriptional units are separated by the 250 cHS4 insulator (SEQ ID NO:358) in the forward orientation.

### Cells Expressing Anti-Idiotype Polypeptides

In some aspects, provided herein are modified cells, and in illustrative embodiments genetically modified cells that include any of the anti-idiotype polynucleotides provided herein. Furthermore, modified cells, or in illustrative embodiments genetically modified cells are provided herein, that expresses any of the anti-idiotype polypeptides provided herein. In some embodiments, the modified or genetically modified cells can further express inhibitory RNA molecules and/or other polypeptides disclosed herein, for example, an engineered signaling polypeptide. In illustrative embodiments, the modified or genetically modified cells can express an anti-idiotype polypeptide and a lymphoproliferative element, CAR, and/or recombinant TCR. In illustrative embodiments, the modified or genetically modified cells can express an anti-idiotype polypeptide and a cytokine. In some embodiments the cytokine is in a secreted form. In some embodiments, the cytokine is membrane-associated. In some embodiments, the cell is an immortalized cell.

In some embodiments, the cells expressing an anti-idiotype polypeptide are lymphocytes, such as TILs, lymphocytes other than B cells, and in illustrative embodiments, T cells and/or NK cells. In some embodiments, the cells express a chimeric TCR or the cells are CAR-T cells and/or CAR-NK cells, or tumor infiltrating lymphocytes. In some embodiments of any of the aspects herein, the T or NK cells are NKT cells. NKT cells are a subset of T cells that express CD3 and typically co-express an αβ T-cell receptor, but also express a variety of molecular markers that are typically associated with NK cells (such as NK1.1 or CD56). In some embodiments, the cells are primary cells. In some embodiments, the cells are human cells. In some embodiments, the cells do not ordinarily produce antibodies. A skilled artisan will understand that reference to a lymphocyte that does not express an antibody in the context of a modified cell that includes or expresses an anti-idiotype polypeptide herein, refers to the native capability of the cell, and that such a modified cell includes, and in illustrative embodiments expresses, an anti-idiotype polypeptide.

In some embodiments the modified cells that have polynucleotides that include nucleic acids that encode an anti-idiotype polypeptide, are present in a cell suspension within a commercial container, such as a commercial container suitable for cell therapy, such as a cell cryopreservation infusion bag. The number of cells in the suspension within the commercial container can be sufficient to provide between 1 × 10⁵ cells and 1 × 10⁹, between 1 × 10⁻⁶ cells and 1 × 10⁹, or between 1 × 10⁻⁶ cells and 5 × 10⁸, for example CAR-positive viable T cells and/or NK cells per kg of body weight of the subject to which the cells are to be delivered. Accordingly, in some embodiments the commercial container, can include the aforementioned ranges × 50-150 kg, or 50-100 kg. In some embodiments, the commercial container includes between 1 × 10⁷ and 1 × 10¹¹ cells, between 1 × 10⁸ and 1 × 10¹¹ cells, or between 1 × 10⁸ and 5 × 10¹⁰ cells, modified cells, for example CAR-positive viable T cells and/or NK cells, and/or in an illustrative embodiment, cells that are positive for an anti-idiotype extracellular recognition domain. As such, anti-idiotype polypeptides herein, can be used to confirm that modified cells that express a CAR, also express the anti-idiotype polypeptide, for example as a release criteria to help assure that the safety switch is present on such cells. Further details regarding modified cells herein, in commercial containers, can be found herein, for non-limiting example, within the “KITS AND COMMERCIAL PRODUCTS” section.

In one aspect, provided herein is a modified or genetically modified T cell or NK cell made using a method according to any of the methods provided herein, wherein the cell is modified to contain a polynucleotide that includes nucleic acids that encode an anti-idiotype polypeptide. In some embodiments, the T cell or NK cell has been further modified to express a first engineered signaling polypeptide. In illustrative embodiments, the first engineered signaling polypeptide can be an LE, a TCR, or a CAR that includes an antigen-specific targeting region (ASTR), a transmembrane domain, and an intracellular activating domain. In some embodiments, the T cell or NK cell can further include a second engineered signaling polypeptide that can be a CAR, a TCR, or a lymphoproliferative element. In some embodiments, the lymphoproliferative element can be a chimeric lymphoproliferative element. In some embodiments, the T cell or NK cell can further include a pseudotyping element on a surface. In some embodiments, the T cell or NK cell can further include an activation element on a surface. The CAR, lymphoproliferative element, pseudotyping element, and activation element of the genetically modified T cell or NK cell can include any of the aspects, embodiments, or sub-embodiments disclosed herein. In illustrative embodiments, the activation element can be anti-CD3 antibody, such as an anti-CD3 scFvFc or an anti-CD3 antibody mimetic.

In some aspects, provided herein are aspects that include a genetically modified and/or transduced T cell or NK cell that include a polynucleotide encoding both an anti-idiotype polypeptide and a self-driving CAR. Details regarding such genetically modified and/or transduced T cells or NK cells, and composition and method aspects including a self-driving CAR, that contain such polynucleotides are disclosed in more detail herein.

In some embodiments, provided herein are genetically modified lymphocytes, in illustrative embodiments TILs, T cells and/or NK cells, or self-driving CAR aspects provided herein, that relate to either aspects for transduction of T cells and/or NK cells in blood or a component thereof, that include transcription units that encode one, two, or more (e.g., 1-10, 2-10, 4-10, 1-6, 2-6, 3-6, 4-6, 1-4, 2-4, 3-4) inhibitory RNA molecules. In some embodiments, such inhibitory RNA molecules are lymphoproliferative elements and therefore, can be included in any aspect or embodiment disclosed herein as the lymphoproliferative element as long as they induce proliferation of a T cell and/or an NK cell, or otherwise meet a test for a lymphoproliferative element provided herein. In some embodiments, inhibitory RNA molecules directed against any of the targets identified in the Inhibitory RNA Molecules section herein.

In some embodiments of the aspect immediately above where the T cell or NK cell comprises one or more (e.g., two or more) inhibitory RNA molecules and the CAR, or nucleic acids encoding the same, the ASTR of the CAR is an MRB ASTR and/or the ASTR of the CAR binds to a tumor associated antigen. Furthermore, in some embodiments of the above aspect, the first nucleic acid sequence is operably linked to a riboswitch, which for example is capable of binding a nucleoside analog, and in illustrative embodiments is an antiviral drug such as acyclovir.

In the methods and compositions disclosed herein, expression of engineered signaling polypeptides is regulated by a control element, and in some embodiments, the control element is a polynucleotide comprising a riboswitch. In certain embodiments, the riboswitch is capable of binding a nucleoside analog and when the nucleoside analog is present, one or both of the engineered signaling polypeptides are expressed. A cell of the current disclosure, for example a lymphocyte, such as a primary cell, and/or a lymphocyte that does not naturally produce antibodies, and in illustrative embodiments, a T cell and/or NK cell, can include more than one type of anti-idiotype polypeptide on its surface, i.e., anti-idiotype polypeptides with different extracellular recognition domains. Thus, in some embodiments, a cell can include a first anti-idiotype polypeptide on its surface that recognizes a first target antibody or antibody mimetic and elicits a first response upon binding, and a second anti-idiotype polypeptide on its surface that recognizes a second target antibody or antibody mimetic and elicits a second response upon binding. For example, addition of the first target antibody or antibody mimetic could activate pro-survival signals in the cell expressing the first anti-idiotype polypeptide through the intracellular domain of the first anti-idiotype polypeptide, and addition of the second target antibody or antibody mimetic could induce ADCC through the antibody or antibody mimetic effector functions. Thus, in some embodiments, a polynucleotide encodes one, two, three, or four, or more anti-idiotype polypeptides. In some embodiments, a cell contains such a polynucleotide, and expresses the one, two, three, or four, or more encoded anti-idiotype polypeptides. A skilled artisan will understand how to combine different anti-idiotype polypeptides to obtain different desired responses.

### Safety Switches

Safety switches have been developed for use with cellular therapies to affect the reduction or elimination of infused cells in the case of adverse events. Any of the replication incompetent recombinant retroviral particles provided herein can include nucleic acids that encode a safety switch as part of, or separate from, nucleic acids encoding any of the engineered signaling polypeptides provided herein. Thus, any of the engineered signaling polypeptides provided herein, for example engineered signaling polypeptides in modified, genetically modified, and/or transduced lymphocytes to be introduced or reintroduced into a subject, can include a safety switch. Thus, any of the engineered T cells disclosed herein can include a safety switch. Safety switch technologies can be broadly categorized into three groups based on their mechanism of action; antibody- or antibody mimetic-mediated cytotoxicity, pro-apoptotic signaling, and metabolic (gene-directed enzyme prodrug therapy, GDEPT). Previously disclosed safety switches include cell surface molecules that are truncated tyrosine kinase receptors. In some of these examples, the truncated tyrosine kinase receptor is a member of the epidermal growth factor receptor (EGFR) family (e.g., ErbB1 (HER1), ErbB2, ErbB3, and ErbB4), for example as disclosed in U.S. Pat. 8,802,374 or WO2018226897. Thus, some of these prior safety switches were polypeptides that are recognized by an antibody that recognizes the extracellular domain of an EGFR member. For example, SEQ ID NO:82, is an exemplary polypeptide that is recognized by, and under the appropriate conditions bound by an antibody that recognizes the extracellular domain of an EGFR member. Such truncated EGFR polypeptides are sometimes referred to herein as eTags. In illustrative embodiments, eTags are recognized by monoclonal antibodies that are commercially available such as matuzumab, necitumumab panitumumab, and in illustrative embodiments, cetuximab. For example, eTag was demonstrated to have a cell killing potential through Erbitux® mediated antibody dependent cellular cytotoxicity (ADCC) pathways. The inventors of the present disclosure have successfully expressed eTag in PBMCs using lentiviral vectors, and have found that expression of eTag in vitro by PBMCs exposed to Cetuximab, provided an effective elimination mechanism for PBMCs. eTags can be used in some embodiments of the present disclosure, but in such embodiments, typically an anti-idiotype extracellular domain is present as well.

In some embodiments, the extracellular recognition domain (i.e. cell tag) is itself an antibody, which as disclosed herein includes a functional antibody fragment, that binds a predetermined binding partner antibody (e.g. a target antibody). In illustrative embodiments, the cell tag antibody is specific for the target antibody, and for example, does not bind antibody constant regions exclusively, or in some embodiments, at all, or in illustrative embodiments, unless they interact with the target antibody (Ab1) to cell tag (extracellular recognition domain) (Ab2) binding. In illustrative embodiments, the cell tag antibody (i.e. extracellular recognition domain that includes the variable region of an antibody) is an anti-idiotypic antibody or antibody mimetic. In some embodiments, the anti-idiotypic antibody (Ab2) recognizes an epitope on the predetermined binding partner antibody (i.e. target antibody) (Ab1) that is distinct from the antigen binding site on Ab1. In illustrative embodiments, Ab2 binds the variable region of Ab1. In other illustrative embodiments, Ab2 binds the antigen-binding site of Ab1, and in illustrative embodiments, competes with Ab1 for binding to the antigen-binding site of Ab1. In certain embodiments, Ab2 may be from any animal including human and murine, or humanized or a chimeric antibody or an antibody derivative included within the definition of “antibody” herein, including, for example antibody fragments (Fab, Fab′, F(ab′)2, scFv, diabodies, bispecific antibodies, and antibody fusion proteins. Ab2 is typically associated with a membrane through a membrane association domain. In certain embodiments, Ab2 is associated with the cell surface via its endogenous transmembrane domain. In other embodiments, Ab2 is associated with the cell surface via a heterologous transmembrane domain or membrane attachment sequence such as GPI. In some embodiments, Ab1 is a commercially available monoclonal antibody. In illustrative embodiments, Ab1 is a commercially available monoclonal antibody therapeutic. In further illustrative embodiments, Ab1 is capable of mediating ADCC and/or CDC as described below. An example of a binding pair comprising an anti-idiotypic antibody (and methods of making the same) displayed on a cell line and cognate monoclonal Ab2 antibodies that mediate ADCC and CDC, is provided in WO2013188864.

In some embodiments, safety switches can also function as flags that label or mark polynucleotides, polypeptides, or cells as being engineered. Such safety switches can be detected using standard laboratory techniques including PCR, Southern Blots, RT-PCR, Northern Blots, Western Blots, histology, and flow cytometry. For example, detection of eTAG by flow cytometry has been used by at least one of the inventors as an in vivo tracking marker for T cell engraftment in mice. In other embodiments, cell tags are used to enrich for engineered cells using antibodies or ligands optionally bound to a solid substrate such as a column or beads. For example, others have shown that application of biotinylated-cetuximab to immunomagnetic selection in combination with anti-biotin microbeads successfully enriches T cells that have been lentivirally transduced with eTAG containing constructs from as low as 2% of the population to greater than 90% purity without observable toxicity to the cell preparation.

In some embodiments provided herein, the anti-idiotype polypeptide is a safety switch (also called a safety switch polypeptide or an anti-idiotype polypeptide safety switch herein) comprising a recognition domain of an anti-idiotype antibody or anti-idiotype antibody mimetic and a membrane association domain. Such safety switch polypeptides can be designed much more efficiently and with many more optional sequences and designs, than prior art safety switches. Such a safety switch polypeptide in one aspect, is designed such that the extracellular recognition domain recognizes an idiotype of an antibody or antibody mimetic capable of inducing cytotoxicity.

Thus, in one aspect the safety switch is based on antibody mediated cytotoxicity upon antibody or antibody mimetic binding to an anti-idiotype polypeptide expressed on the surface of a cell, and more specifically binding to an extracellular recognition domain (also referred to herein as a cell tag or more specifically, an anti-idiotype cell tag) of an anti-idiotype polypeptide. In some embodiments, the antibody or antibody mimetic binds to the cell tag and induces complement-dependent cytotoxicity (CDC) and/or antibody-dependent cell-mediated cytotoxicity (ADCC). In some embodiments, binding of the antibody or antibody mimetic to the anti-idiotype polypeptide induces, promotes, and/or activates one or more of ADCC, CDC, antibody-mediated complement activation, antibody-dependent cellular phagocytosis, and antibody-dependent enhancement of diseases. Details related to other antibody and antibody mimetic functions, including corresponding Fc domains for eliciting such responses, are discussed in the “Antibody and antibody mimetic effector functions” herein. The anti-idiotype polypeptide can be immunogenic, to further stimulate the immune system. Thus, in some embodiments, the cell tag is immunogenic. In other embodiments, the cell tag polypeptide is non-immunogenic.In another aspect, a safety switch polypeptide is designed such that the anti-idiotype polypeptide includes an intracellular domain having one or more cell-death inducing signals, and the polypeptide is capable of inducing a cell death signal upon binding of the anti-idiotype polypeptide to a target antibody or antibody mimetic that comprises the idiotype recognized by the anti-idiotype polypeptide. The cell-death inducing signals can be induced based on dimerization-induced apoptotic signaling. In some embodiments, the safety switch is based on dimerization induced apoptotic signals. In some embodiments, such a safety switch comprises an extracellular dimerization domain comprising a recognition domain of an anti-idiotype antibody or antibody mimetic linked in frame with a membrane association domain and an intracellular domain comprising components of an apoptotic pathway. Thus, dimerization mediated by the binding of an antibody or antibody mimetic to the anti-idiotype polypeptide results in apoptosis of the cell. In some embodiments, the safety switch includes inducible FAS (iFAS) comprised of one or more inducible dimerization domains, i.e., the anti-idiotype polypeptides, fused to the cytoplasmic tail of the Fas receptor and localized to the membrane by a membrane association domain. As discussed in the “Intracellular domains” section herein, in some embodiments, the safety switch includes one or more domains from a Caspase, such as caspase-1 or caspase-9.

As discussed in the “Anti-idiotype fusion polypeptides” section herein, the anti-idiotype polypeptides, including the safety switches discussed in this section, can be expressed as fusions with other polypeptides disclosed herein, including a lymphoproliferative element, a CAR, and/or a recombinant TCR. In other embodiments, the anti-idiotype polypeptides are expressed as polypeptides by themselves. In any of these embodiments, the anti-idiotype polypeptides can include any of the domains disclosed herein to be included in a lymphoproliferative element, CAR, and/or TCR, such as the extracellular domains, stalks, transmembrane domains, intracellular activating domains, modulatory domains, linkers, or intracellular domains.

### Anti-Idiotype Methods

The anti-idiotype compositions provided herein have many uses, including in in vitro and in vivo methods for cell tagging for example, and in illustrative embodiments, in methods of delivering polynucleotides or polynucleotide vectors (e.g., RIPs) and/or modified cells to a subject. Such methods for delivering polynucleotides, polynucleotide vectors and/or modified cells to a subject can for example, utilize signaling that is regulated by binding of a target antibody to an anti-idiotype extracellular recognition domain of an anti-idiotype polypeptide provided herein. Such a method can be a method for inducing target cell proliferation or death, wherein the target cells are the modified cells that are delivered, or are cells present in the subject that are modified in vivo in the subject by transduction, transfection, electroporation, or other means of gene delivery, with polynucleotides or polynucleotide vectors encoding anti-idiotype polynucleotides that are directly delivered in some embodiments. Target cell proliferation or death in illustrative embodiments, is induced by binding of a target antibody to an anti-idiotype extracellular recognition domain expressed on the target cell.

Thus, in one aspect, provided herein is a method for administering modified cells and/or polynucleotides, such as polynucleotide vectors, that include nucleic acids that encode an anti-idiotype polypeptide to a mammalian subject. The method of this aspect includes delivering the polynucleotides, such as polynucleotide vectors and/or modified cells to the mammalian subject. The polynucleotides or polynucleotide vectors can be any of the polynucleotide vectors provided herein that comprise nucleic acids encoding an anti-idiotype polypeptide provided herein. Such anti-idiotype polypeptides in certain embodiments include a membrane association domain, which in illustrative embodiments is separated from the anti-idiotype extracellular recognition domain on the anti-idiotype polypeptide, by a stalk. In some embodiments the anti-idiotype polypeptide includes an intracellular domain, which in some embodiments is an intracellular signaling domain. The modified cells can be any of the modified cells provided herein that comprise, and in illustrative embodiments express, any of the polynucleotides provided herein that comprise nucleic acids that encode an anti-idiotype polypeptide.

In illustrative embodiments, the modified cells are genetically modified cells. Such modified cells, or genetically modified cells, in some non-limiting embodiments are lymphocytes or lymphocytes other than B cells, and in some embodiments T cells and/or NK cells, such as CAR-T cells and/or CAR-NK cells. In certain embodiments, the polynucleotides are RNA or DNA, and in illustrative embodiments are mRNA, for example a synthetic anti-idiotype mRNA as disclosed herein. In illustrative embodiments, the polynucleotide vectors are replication incompetent retroviral particles (RIPs), for example recombinant lentiviral particles.

Systems and methods for processing cells that are removed from the subject, such as methods for isolating and modifying blood cells, for example PBMCs, can include traditional closed cell processing systems and methods, or “more recent” methods and systems as disclosed in further detail herein. Accordingly, provided herein in certain aspects, in addition to a method for administering modified cells, or as a part of or in combination with such a method (usually before the delivery step of such method for administering), is a method of transducing, modifying, and/or genetically modifying, peripheral blood mononuclear cells (PBMCs), or lymphocytes, typically T cells and/or NK cells, and in certain illustrative embodiments resting T cells and/or resting NK cells, in illustrative embodiments with polynucleotides that include nucleic acids encoding anti-idiotype polypeptides, in a reaction mixture. Such methods can include a contacting step that includes contacting lymphocytes with polynucleotide vectors, such as, but not limited to, replication incompetent recombinant retroviral particles (RIPs) in the reaction mixture, that include nucleic acids that encode an anti-idiotype polypeptide. Such reaction mixture, themselves represent separate aspects provided herein. In some embodiments, the reaction mixture comprises blood, or a component thereof, and/or an anticoagulant. The reaction mixture in illustrative embodiments comprises lymphocytes and identical copies of polynucleotide vectors, such as a replication incompetent recombinant retroviral particles, that comprise nucleic acids that encode an anti-idiotype polypeptide provided herein. Furthermore, the reaction mixture can include a T cell activation element. In certain embodiments, the reaction mixture includes one or more additional blood components set out below that in illustrative embodiments are present because the reaction mixture comprises at least 10% whole blood. In illustrative embodiments, the RIPs comprise a T cell binding polypeptide and a fusogenic polypeptide on their surface, which in illustrative embodiments is a pseudotyping element. In such methods, the contacting (and incubation under contacting conditions) facilitates association of the lymphocytes with the RIPs, wherein the RIPs genetically modify and/or transduce the lymphocytes. In certain illustrative embodiments that especially related to “more recent” methods for processing T cells, which can be performed in much shorter times, as discussed herein, the RIPs can further include a T cell activation element on their surface.

In some embodiments, the methods further include instructing a medical professional, patient or caregiver that a target antibody should be administered to the subject if life-threatening adverse events develop, and/or delivering a target antibody or antibody mimetic to the subject in response to life-threatening adverse events. Such adverse events include grade 3 and/or grade 4 adverse events, including grade 3 and/or grade 4 CRS or ICANS (Lee et al, “ASTCT Consensus Grading for Cytokine Release Syndrome and Neurologic Toxicity Associated with Immune Effector Cells”; Biology of Blood and Marrow Transplantation; 25: 6235-638 (2019)). The target antibody or antibody mimetic in illustrative embodiments, comprises an idiotype recognized by the anti-idiotype extracellular recognition domain. The target antibody or antibody mimetic can be any of the target antibodies and antibody mimetics disclosed herein, including, but not limited to, approved biologic target antibodies and antibody mimetics. The target antibody or antibody mimetic in some embodiments, is delivered in sufficient quantity to selectively kill at least 1%, 10%, 15%, 20%, 25%, 30%, 40%, 50%, 60%, 70%, 75%, 80%, 90%, 95%, or 99% of the genetically modified T cells and/or NK cells in the subject.

Methods for delivering modified cells, polynucleotides, polynucleotide vectors, and antibody or antibody mimetics to a subject are known in the art. In some embodiments of any of the aspects herein, delivery of the modified or genetically modified cells, polynucleotides or the polynucleotide vectors (e.g., RIPs), or the target antibody or antibody mimetic is performed by intravenous administration/infusion, intraperitoneal administration/injection, subcutaneous administration/injection, or intramuscular administration/injection. In some embodiments, the modified cells (e.g., lymphocytes) introduced into the subject are autologous cells, and in other embodiments they are allogeneic cells. In embodiments utilizing allogeneic cells, typically the cells (e.g., lymphocytes) are from a different person, and the cells (e.g., lymphocytes) from the subject are not modified. In some embodiments, no blood is collected from the subject to harvest cells during the method.

The present disclosure provides various treatment methods that involve delivery of an engineered T cell receptor or a CAR. An engineered T cell receptor or a CAR of the present disclosure, when present in a modified T lymphocyte or NK cell, or in a polynucleotide vector to be delivered directly to a subject, can mediate cytotoxicity toward a target cell. Such methods typically involve administration of modified lymphocytes, or substantially purified or purified polynucleotide vectors (e.g., RIRs) to a subject as provided herein. An engineered T cell receptor or CAR binds to an antigen present on a target cell, thereby mediating killing of a target cell by a T lymphocyte or an NK cell genetically modified to produce the engineered T cell receptor or CAR. In order to improve the effectiveness and/or the safety of such methods, in certain illustrative embodiments, modified T cells and/or NK cells, polynucleotides, or polynucleotide vectors that are delivered into a subject, are further modified such that they are capable of expressing, or express an anti-idiotype polypeptide and a CAR, a TCR, a cytokine, and/or a lymphoproliferative element as provided herein. In some embodiments, the modified cells express the anti-idiotype polypeptides and one or more of the CAR, the TCR, the cytokine, and/or the lymphoproliferative element from the same polynucleotide and sometimes on the same polypeptide, as disclosed herein. In illustrative embodiments, the modified cells are lymphocytes, in illustrative embodiments T cells and/or NK cells. In certain illustrative embodiment, such methods herein include delivery directly into a subject, of polynucleotides and polynucleotide vectors provided herein that include a promoter that is active in lymphocytes, in illustrative embodiments active in T cells and/or NK cells, and encode an anti-idiotype polypeptide.

Such methods that involve delivery of modified T cells and/or NK cells, or polynucleotide or polynucleotide vectors with promoters that are active in T cells and/or NK cells, are especially suited for, adapted for, and/or effective for methods in which the anti-idiotype polypeptides regulate T cell and/or NK cell proliferation (e.g., are useful as lymphoproliferative elements) and/or inducible cell killing (e.g., are useful as safety switches). Methods herein where anti-idiotype polypeptides are used to promote selective killing of modified cells, for example modified CAR-T, CAR-NK, or TIL cells, can be referred to herein as safety switch methods. In illustrative embodiments, such safety switch methods utilize any of the polynucleotides that include nucleic acids encoding anti-idiotype polynucleotides, and corresponding encoded and in illustrative embodiments, expressed, anti-idiotype polypeptides, provided herein. In some embodiments, such safety switch methods involve delivery of modified CAR-T cells, modified CAR-NK cells, or modified TIL cells to the subject, and can involve delivery of a target clinical antibody. Antibody delivery can be directed and administered to induce proliferation of the modified cells in the subject, and/or if the subject develops clinical symptoms that indicate that the modified cells that were delivered or cells derived therefrom, are causing adverse events in the subject, for example, grade 1 or grade 2 adverse events, and in some embodiments grade 3 and grade 4 adverse events, or otherwise putting the subject at risk of medical complications or even death. Thus, in some aspects, provided herein is a method, system, and kits, wherein the same anti-idiotype polypeptide, or polynucleotide, which typically for these embodiments includes an intracellular proliferation-inducing signaling domain, can be used to induce proliferation or killing, depending on whether the antibody that contacts a modified cell expressing the anti-idiotype polypeptide, has a structure to induce cell killing or only dimerization of anti-idiotype polypeptides. Typically, especially in safety switch methods, delivery of the target clinical antibody would be at a later date that is days, weeks, or months after the first or most recent delivery of the modified cells to the subject. In some embodiments, methods for administering a modified cell, such as a modified T cell and/or NK cell, can include receiving information regarding adverse events of a subject and/or testing a subject for adverse events, and instructing a medical professional, a patient, and/or a caregiver to administer a target antibody if a patient develops certain adverse events, and in some embodiments such target antibody is administered. For example, the instructions can specify to administer the target antibody if the patient develops certain grade 3 or grade 4 adverse events. Such adverse events can include, but are not limited to tumor lysis syndrome, cytokine release syndrome (CRS), macrophage activation syndrome, and/or neurotoxicity. In some embodiments herein, levels of interferon (IFN)-γ, IL-2, soluble IL-2Rα, IL-6, soluble IL-6R and granulocyte-macrophage colony-stimulating factor (GM-CSF), IL-1,IL-6, IL-8,IL-10,IL-12, tumor necrosis factor (TNF)-α, IFN-α, monocyte chemotactic protein (MCP)-1, macrophage inflammatory protein (MIP) 1α, can be detected and in illustrative embodiments measured to identified CRS. To detect neurotoxicity, some embodiments herein include detecting and/or receiving a report of decreased level of consciousness, confusion, seizures and cerebral edema.

A method of treating a disease or disorder in a subject having the disease or disorder, can include. contacting a polynucleotide vector, such as an expression vector, including nucleic acid sequences encoding an anti-idiotype polypeptide provided herein, with peripheral blood cells obtained from the subject to produce modified T cells and/or NK cells, such as modified cytotoxic T cells and/or NK cells, before administering the modified T cells and/or NK cells to the subject.

In some embodiments, methods provided herein, such as methods for delivering modified cells to a subject, which can be adoptive cell therapies, methods for producing persistent populations of cells, methods for delivering a formulation, etc. as non-limiting examples, are especially adopted for treating cancer. Such cancer can be any type of cancer. For example, such methods can be used for treating a subject who has, or a tumor associated with ovarian cancer, soft tissue sarcoma, peripheral T cell cancer, colorectal cancer, intrahepatic cholangiocarcinoma, glioblastoma, esophageal cancer, cutaneous T cell lymphoma, non-hodgkin lymphoma, urothelial cancer, basal cell carcinoma, epithelioid sarcoma, pancreatic cancer, non-small cell lung carcinoma, hodgkin lymphoma, renal cell carcinoma, mesothelioma, metastatic uveal melanoma, kidney cancer, blood cancer, HER2-expressing cancers, non-melanoma skin cancer, liposarcoma, hepatocellular carcinoma, small lymphocytic lymphoma, prostate cancer, breast cancer, anal cancer, marginal zone lymphoma, cutaneous squamous cell carcinoma, thyroid cancer, medullary thyroid cancer, triple-negative breast cancer, neuroendocrine prostate cancer, bladder cancer, paraganglioma, medulloblastoma, superficial basal cell carcinoma, head and neck squamous cell carcinoma, hematologic malignancies, melanoma, B-cell lymphoma, relapsed/refractory acute myeloid leukemia, angiosarcoma, bone sarcoma, refractory cervical cancer, cholangiocarcinoma, osteosarcoma, biliary tract cancer, castration-resistant prostate cancer, gastroesophageal adenocarcinomas, rhabdomyosarcoma, carcinoma, non-muscle invasive bladder cancer, uveal melanoma, small cell lung cancer, cervical cancer, primary open angle glaucoma, follicular lymphoma, synovial sarcoma, liver cancer, carcinosarcoma, leptomeningeal brain tumors, T-cell lymphoma, lymphoma, small cell lung cancer, mantle cell lymphoma, B-cell malignancies, endometrial cancer, myxoid/round cell liposarcoma, metastatic merkel cell carcinoma, neuroblastoma, chronic lymphocytic leukemia, tenosynovial giant cell tumors, sarcoma, acute myeloid leukemia, skin cancer, nasopharyngeal carcinoma, relapsed/refractory ewing sarcoma, bone cancer, glioma, salivary gland carcinoma, gastric cancer, benign tumor, low-grade serous ovarian cancer, metastatic breast cancer, multiple myeloma, diffuse large B cell lymphoma, relapsed/refractory lymphoma, metastatic colorectal cancer, advanced malignancies, acute lymphoblastic leukemia, mesothelin-expressing solid tumors.

In some embodiments, methods herein can be used to treat tumors that express any one or more of the tumor-associated antigens and/or tumor-specific antigens provided herein, and engineered T cell receptors and CARs can be designed to recognize such targets. As non-limiting examples, such tumor associated or tumor specific antigens include blood tumor antigens provided herein elsewhere in this specification, and in some non-limiting embodiments includes the following antigens, most or all of which are believed to be associated with solid tumors: AXL, CD44v6, CAIX, CEA, CD133, c-Met, EGFR, EGFRvIII, Epcam, EphA2, GD2, GPC3, GUCY2C, HER1, HER2, ICAM-1, IL13Rα2, IL11Ra, Kras, Kras G12D, L1CAM, MAGE, MET, Mesothelin, MUC1, MUC16 ecto, NKG2D, NY-ESO-1, PSCA, ROR-2, WT-1.

In some embodiments, any of the methods provided herein that involve an administering step, can be combined with administration of another cancer therapy, which in certain embodiments, can be a cancer vaccine, for example delivered subcutaneously. In other embodiments and optionally in further combination with cancer vaccine administration, such methods provided herein that include administering genetically modified T cells and/or NK cells into a subject, especially where the subject has, is afflicted with, or is suspected of having cancer, can further include delivering an effective dose of an immune checkpoint inhibitor to the subject. This checkpoint inhibitor delivery can occur before, after, or at the same time as administering the genetically modified T cells and/or NK cells. Immune checkpoint inhibitors are known and various compounds are approved and in clinical development. Check point molecules, many of which are the target of checkpoint inhibitor compounds, include, but not limited to an anti-PD1 antibody.

In some embodiments, the administering is for treating cancer in the subject, and wherein a tumor in the subject regresses within 60 days, 45 days, 30 days, or 14 days after said administering. In some embodiments, the tumor is a blood cancer, for example DLBCL, that in illustrative examples expresses any of the blood cancer antigens provided herein. In other embodiments, the tumor is a solid tumor that expresses a solid tumor antigen, which in certain illustrative embodiments is a HER2 positive solid tumor, such as, but not limited to, breast cancer. In some embodiments, the administering is for treating cancer in the subject, and wherein the subject experiences stable disease, at least a partial response, or a complete response, in illustrative embodiments by RECIST1.1 criteria, within 90 days, 75 days, 60 days, 45 days, 30 days, or 14 days after said administering. In some embodiments, the tumor shrinks by at least 10%, 20%, 25%, 30%, 50% or more. In some embodiments, a partial response occurs when the sum of tumor lesions reduces by 30% or more and is confirmed at least 4 weeks after the prior scan without the appearance of new lesions and/or any pathological lymph nodes have a reduction in short axis to less than 10 mm. In some embodiments, a complete response occurs when all target and non-target lesions disappear. In some embodiments, the administering is for treating cancer in the subject, and wherein the subject experiences at least a partial response or experiences a complete response within 60 days, 45 days, 30 days, or 14 days after said administering. In some embodiments, the subject is a human afflicted with cancer. In some embodiments, the cell formulation is administered 2, 3, 4, 5, 6, or more times, or in illustrative embodiments only once to the subject before stable disease, or in illustrative embodiments a partial response or a complete response is achieved. In some embodiments, a second formulation is administered to the subject at a second, third, fourth, etc. timepoint between 1 day and 1 month, 2 months, 3 months, 6 months, or 12 months after the administering a first cell formulation, wherein the second formulation can be identical to the first formulation, or can comprises any of the formulations provided herein.

In any of the aspects provided herein that include delivery of modified lymphocytes (e.g. modified T cells and/or NK cells), the method can be performed on a mammalian subject that has been subjected to a lymphodepletion process, as are known in the art. However, in illustrative embodiments, the administration of the modified T cells and/or NK cells, or RER retroviral particles (RIPs), is performed in a method that does not require lymphodepletion of the subject for successful engraftment in the subject and/or for successful reduction of tumor volume in the subject, or that is performed on a mammalian (e.g. human) subject that has not been subjected to lymphodepletion in the prior days, weeks, or months or ever before such administration (e.g. subcutaneous administration). In certain embodiments, the administration is performed on a mammalian (e.g. human) subject that is not suffering from a low white blood cell count, lymphopenia or lymphocytopenia. In certain embodiments, the subcutaneous administration is performed on a subject having a lymphocyte count in the normal range (i.e., 1,000 and 4,800 lymphocytes in 1 microliter (µL) of blood). In certain embodiments, the subcutaneous administration is performed on a subject having between 1,000 and 5,000, over 300, over 500, over 1,000, over 1,500, or over 2,000 lymphocytes per µL of blood). In certain embodiments, the subcutaneous administration is performed on a mammalian (e.g., human) subject that is lymphoreplete.

Further details regarding steps and systems for performing anti-idiotype methods can be found throughout this disclosure, included as a non-limiting example, the section entitled “STEPS AND

### Reaction Mixtures for Methods Herein″

### Target Antibodies and Antibody Mimetics

Anti-idiotype polypeptides of the current disclosure bind to the idiotype of target antibodies and/or target antibody mimetics. In some embodiments, the target antibody can include one or more domains from or derived from a a mouse antibody, a rat antibody, a rabbit antibody, a goat antibody, a chicken antibody, a sheep antibody, a cow antibody, a llama antibody, a chimeric antibody, or in illustrative embodiments, a human antibody. In some embodiments, different target antibodies or target antibody mimetics can be used to bind to the same anti-idiotype polypeptide to elicit different responses, for example a first target antibody to promote cell proliferation and/or survival and a second target antibody mimetic to promote cell death.

In some embodiments, the target antibody or antibody mimetic is a therapeutic antibody or a therapeutic antibody mimetic, respectively. In some embodiments, the target antibody or antibody mimetic can be a clinical antibody or clinical antibody mimetic, respectively. In some embodiments, the clinical antibody or clinical antibody mimetic is the subject of an FDA-approved Investigational New Drug Application (IND), or equivalent approved regulatory filing for initial clinical testing in humans in another country or jurisdiction. In some embodiments, the target antibody or the target antibody mimetic as per the Investigational New Drug Application or equivalent is a stand-alone product, with no other active therapeutic or ingredient being tested as part of the IND. In some illustrative embodiments, the clinical antibody or antibody mimetic is a regulatory agency (e.g., U.S. FDA) approved biologic. In some embodiments, the target antibody can be one or more of cetuximab, muromonab-CD3, efalizumab, tositumomab-i131, nebacumab, edrecolomab, catumaxomab, daclizumab, olaratumab, abciximab, rituximab, basiliximab, palivizumab, infliximab, trastuzumab, adalimumab, ibritumomab tiuxetan, omalizumab, bevacizumab, natalizumab, panitumumab, ranibizumab, eculizumab, certolizumab pegol, ustekinumab, canakinumab, golimumab, ofatumumab, tocilizumab, denosumab, belimumab, ipilimumab, brentuximab vedotin, pertuzumab, ado-trastuzumab emtansine, raxibacumab, obinutuzumab, siltuximab, ramucirumab, vedolizumab, nivolumab, pembrolizumab, blinatumomab, alemtuzumab, evolocumab, idarucizumab, necitumumab, dinutuximab, secukinumab, mepolizumab, alirocumab, daratumumab, elotuzumab, ixekizumab, reslizumab, bezlotoxumab, atezolizumab, obiltoxaximab, brodalumab, dupilumab, inotuzumab ozogamicin, guselkumab, sarilumab, avelumab, emicizumab, ocrelizumab, benralizumab, durvalumab, gemtuzumab ozogamicin, erenumab (erenumab-aooe), galcanezumab (galcanezumab-gnlm), burosumab (burosumab-twza), lanadelumab (lanadelumab-flyo), mogamulizumab (mogamulizumab-kpkc), tildrakizumab (tildrakizumab-asmn), fremanezumab (fremanezumab-vfrm), ravulizumab (ravulizumab-cwvz), cemiplimab (cemiplimab-rwlc), ibalizumab (ibalizumab-uiyk) emapalumab (emapalumab-lzsg), moxetumomab pasudotox (moxetumomab pasudotox-tdfk), caplacizumab (caplacizumab-yhdp), risankizumab (risankizumab-rzaa), polatuzumab vedotin (polatuzumab vedotin-piiq), romosozumab (romosozumab-aqqg), brolucizumab (brolucizumab-dbll), crizanlizumab (crizanlizumab-tmca), enfortumab vedotin (enfortumab vedotin-ejfv), [fam-]trastuzumab deruxtecan (fam-trastuzumab deruxtecan-nxki), teprotumumab (teprotumumab-trbw), eptinezumab (eptinezumab-jjmr), isatuximab (isatuximab-irfc), sacituzumab govitecan (sacituzumab govitecan-hziy), inebilizumab (inebilizumab-cdon), tafasitamab (tafasitamab-cxix), belantamab mafodotin (belantamab mafodotin-blmf), satralizumab (satralizumab-mwge), atoltivimab, maftivimab, odesivimab-ebgn, naxitamab-gqgk, margetuximab-cmkb, ansuvimab-zykl, evinacumab, dostarlimab (dostarlimab-gxly), loncastuximab tesirine (loncastuximab tesirine-lpyl), amivantamab (amivantamab-vmjw), aducanumab (aducanumab-avwa), tralokinumab, anifrolumab (anifrolumab-fnia), oportuzumab monatox, tisotumab vedotin, bimekizumab, narsoplimab, tezepelumab, sintilimab, inolimomb, balstilimab, ublituximab, toripalimab, omburtamab, penpulimab, tanezumab, faricimab, sutimlimab, teplizumab, and retifanlimab. In illustrative embodiments, binding of the anti-idiotype polypeptide to its target antibody or antibody mimetic prevents and/or blocks binding of the target antibody or antibody mimetic to the cognate antigen of the target antibody or antibody mimetic. In some embodiments, the extracellular recognition domain of the anti-idiotype polypeptide is capable of, adapted for, and/or configured to prevent and/or block binding of the target antibody or antibody mimetic to the cognate antigen of the target antibody or antibody mimetic when the anti-idiotype polypeptide is bound to the target antibody or antibody mimetic. In illustrative embodiments, the target antibody is cetuximab. In further illustrative embodiments, binding of the anti-idiotype polypeptide to cetuximab prevents and/or blocks binding of cetuximab to Epidermal Growth Factor Receptor (EGFR). In some embodiments, the extracellular recognition domain of the anti-idiotype polypeptide is capable of, adapted for, and/or configured to prevent and/or block binding of cetuximab to EGFR when the anti-idiotype polypeptide is bound to cetuximab.

In some embodiments, the clinical antibody or clinical antibody mimetic has been shown in one or more clinical trials to have an acceptable safety (i.e., adverse event) profile. In some embodiments, the clinical antibody or clinical antibody mimetic has passed human clinical safety testing in a stand-alone clinical trial of the clinical antibody or clinical antibody mimetic. In some embodiments, the clinical antibody or clinical antibody mimetic for which a stand-alone application for regulatory approval has been filed with the Food and Drug Administration of the U.S. (USFDA), European Medicines Agency (EMA), National Medical Products Administration of China (NMPA) (Chinese FDA), or the Pharmaceutical and Food Safety Bureau (PFSB) of Japan. In some embodiments, the clinical antibody or clinical antibody mimetic for which an application for approval (e.g., Biologic License Application (BLA)) has been filed with the Food and Drug Administration of the U.S. (USFDA), European Medicines Agency (EMA), National Medical Products Administration of China (NMPA) (Chinese FDA), or the Pharmaceutical and Food Safety Bureau (PFSB) of Japan. In some embodiments, the clinical antibody or clinical antibody mimetic is an approved biologic antibody or antibody mimetic, approved by the Food and Drug Administration of the U.S. (USFDA), European Medicines Agency (EMA), National Medical Products Administration of China (NMPA) (Chinese FDA), or the Pharmaceutical and Food Safety Bureau (PFSB) of Japan.

In some embodiments, the target antibody or antibody mimetic can be a bispecific antibody. In some embodiments, the target antibody or antibody mimetic can be a multispecific antibody. Multispecific antibodies have binding specificities for at least two different sites. In some embodiments, one of the binding specificities is for one target antigen and the other is for another target antigen. In some embodiments, bispecific antibodies or antibody mimetics may bind to two different epitopes of a target antigen. In some embodiments, one of the binding specificities is for one target antigen, which is not an antibody or antibody mimetic, and the other is for a second target antigen, which is an antibody or antibody mimetic.

In some embodiments, the target antibody or antibody mimetic recognized by an anti-idiotype polypeptide includes an Fc domain from IgM, IgD, IgG, IgA, or IgE. In some embodiments, the antibody or antibody mimetic recognized by an anti-idiotype polypeptide includes an Fc domain from IgM, IgD, IgG1, IgG2, IgG3, IgG4, IgA1, IgA2, or IgE. In some embodiments, a target antibody or antibody mimetic with an IgM Fc domain can be used to drive higher order multimerization.

In some embodiments, the target antibody or antibody mimetic includes glycosylated residues. In some embodiments, the target antibody or antibody mimetic includes an α-Gal epitope. In some embodiments, the target antibody or antibody mimetic includes one or more glycoforms. In some embodiments, the glycoforms or antibody mimetic includes the target antibody with an α-1,3-Gal residue. In some embodiments, the glycoforms comprise the target antibody with an N-glycolylneuraminic acid residue. In some embodiments, the glycoforms comprise the target antibody with an oligomannose.

The cell line an antibody or antibody mimetic is produced in can affect glycosylation of the residues in the polypeptide. In some embodiments, the target antibody or antibody mimetic can be produced in a CHO cell line, a BHK cell line, an NS0 cell line, an SP2/0 cell line, a YB2/0 cell line, or an HEK293 cell line or its derivatives (e.g., a HT-1080 cell line, a Huh-7 cell line, or a PER.C6 cell line). In illustrative embodiments, the target antibody or antibody mimetic can be produced in a CHO cell line. In other illustrative embodiments, the target antibody or antibody mimetic can be produced in an NS0 cell line. In other illustrative embodiments, the target antibody or antibody mimetic can be produced in an SP2/0 cell line.

In some embodiments, the anti-idiotype polypeptide recognizes a target antibody mimetic. In some embodiments, the target antibody mimetic can be an afflilin, an affimer, an affitin, an alphabody, an alphamab, an anticalin, a peptide aptamer, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a nanofitin, a leucine-rich repeat domain, a lipocalin domain, a mAb 2 or Fcab™, a nanobody, a nanoCLAMP, an OBody, a Pronectin, a single-chain TCR, a tetratricopeptide repeat domain, VHH, or a V-like domain. Antibody mimetic can include Fc domains to control the effect of binding of the anti-idiotype antibody mimetic to the target antibody mimetic, as discussed in the “Antibody and antibody mimetic effector functions” section.

### Antibody Variants

In any of the embodiments disclosed herein, the amino acids in the polypeptide sequences of the extracellular recognition domain of an anti-idiotype polypeptide or of the antibodies and antibody mimetics to which the anti-idiotype polypeptide binds can contain substitutions or variations. The anti-idiotype polypeptides, antibodies and antibody mimetics obtained by performing substitutions or variations in the amino acid sequences can be referred to as anti-idiotype polypeptide variants, antibody variants and antibody mimetic variants, respectively. In some embodiments, the antibodies and antibody mimetics to which the anti-idiotype polypeptide binds, can be antibody variants and antibody mimetic variants, respectively. Anti-idiotype polypeptide variants, antibody variants, and antibody mimetic variants can be prepared by introducing appropriate changes into the nucleotide sequence of the nucleic acids encoding the antibody or antibody mimetic. Alternatively, antibody variants and antibody mimetic variants can be prepared by peptide synthesis. Such modifications include, for example, deletions, insertions, and/or substitutions of residues within the amino acid sequences of the anti-idiotype polypeptide, antibody, or antibody mimetic. Any combination of deletion, insertion, and substitution can be made to generate the anti-idiotype polypeptide variant, antibody variant, or antibody mimetic variant, provided that the anti-idiotype polypeptide variant, antibody variant, or antibody mimetic variant possesses the desired characteristics, for example, idiotype or antigen binding. Not to be limited by theory, the variations can be introduced into the antibody in order to improve the binding affinity, and/or other biological properties of the antibody. In some embodiments, the variants include one or more amino acid substitutions.

In some embodiments, an alanine (Ala) residue can be substituted with valine (Val), leucine (Leu), or isoleucine (Ile). In some embodiments, an arginine (Arg) residue can be substituted with lysine (Lys), glutamine (Gln), or asparagine (Asn). In some embodiments, an asparagine (Asn) residue can be substituted with glutamine (Gln), histidine (His), aspartic acid (Asp), lysine (Lys), or arginine (Arg). In some embodiments, an aspartic acid (Asp) residue can be substituted with glutamic acid (Glu) or asparagine (Asn). In some embodiments, a cysteine (Cys) residue can be substituted with serine (Ser) or alanine (Ala). In some embodiments, a glutamine (Gln) residue can be substituted with asparagine (Asn) or glutamic acid (Glu). In some embodiments, a glutamic acid (Glu) residue can be substituted with aspartic acid (Asp) or glutamine (Gln). In some embodiments, a glycine (Gly) residue can be substituted with alanine (Ala). In some embodiments, a histidine (His) residue can be substituted with asparagine (Asn), glutamine (Gln), lysine (Lys), or arginine (Arg). In some embodiments, an isoleucine (Ile) residue can be substituted with leucine (Leu), valine (Val), methionine (Met), alanine (Ala), phenylalanine (Phe), or norleucine. In some embodiments, a leucine (Leu) residue can be substituted with norleucine, isoleucine (Ile), valine (Val), methionine (Met), alanine (Ala), or phenylalanine (Phe). In some embodiments, a lysine (Lys) residue can be substituted with arginine (Arg), glutamine (Gln), or asparagine (Asn). In some embodiments, a methionine (Met) residue can be substituted with leucine (Leu), phenylalanine (Phe), or isoleucine (Ile). In some embodiments, a phenylalanine (Phe) residue can be substituted with tryptophan (Trp), leucine (Leu), valine (Val), isoleucine (Ile), alanine (Ala), or tyrosine (Tyr). In some embodiments, a proline residue can be substituted with alanine (Ala). In some embodiments, a serine (Ser) residue can be substituted with threonine (Thr). In some embodiments, a threonine (Thr) residue can be substituted with valine (Val) or serine (Ser). In some embodiments, a tryptophan (Trp) can be substituted with tyrosine (Tyr) or phenylalanine (Phe). In some embodiments, a tyrosine (Tyr) residue can be substituted with tryptophan (Trp), phenylalanine (Phe), threonine (Thr), or serine (Ser). In some embodiments, a valine (Val) residue can be substituted with isoleucine (Ile), leucine (Leu), methionine (Met), phenylalanine (Phe), alanine (Ala), or norleucine.

As discussed elsewhere herein, the target antibody or antibody mimetic can include an Fc region. Not to be limited by theory, glycosylation of the Fc region of the target antibody or antibody mimetic can affect the antibody effector function. In some embodiments, the antibody or antibody mimetic including an Fc region can be altered by altering the carbohydrate attached to the Fc region. Native antibodies produced by mammalian cells typically include a branched, biantennary oligosaccharide that is typically attached by an N-linkage to Asn297 of the CH2 domain of the Fc region. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody or antibody mimetic of the invention can be made to generate antibody or antibody mimetic variants with certain improved properties. In some embodiments, antibody or antibody mimetic variants include a carbohydrate structure that lacks fucose directly or indirectly attached to an Fc region. In some embodiments, the amount of fucose in the antibody or antibody mimetic variants may be from 1% to 80%, 1% to 65%, 5% to 65%, or 20% to 40%. The amount of fucose can be determined by calculating the average amount of fucose within the sugar chain at Asn 297, relative to the sum of all glycostructures attached to Asn 297 (e.g., complex, hybrid and high mannose structures) as measured by MALDI-TOF mass spectrometry, as described in WO2008/077546, for example. Asn297 refers to the asparagine residue located at about position 297 in the Fc region (Eu numbering of Fc region residues); however, Asn297 can also be located within about 3 amino acids upstream or downstream of position 297, i.e., between positions 294 and 300, due to minor sequence variations in antibodies. Such fucosylation variants may have improved ADCC function. See, for example, U.S. Pat. Publication Nos. US2003/0157108; US2004/0093621.

In some embodiments, the antibody or antibody mimetic variants can include bisected oligosaccharides in which a biantennary oligosaccharide attached to the Fc region of the antibody is bisected by N-acetylglucosamine (GlcNAc). The bisecting GlcNAc structure is a β1,4-linked GlcNAc attached to the core β-mannose residue, representing a special type of N-glycosylated modification. GlcNAc transferred to the 4-position of the β-linked core mannose (Man) residue in complex or hybrid N-glycans by the β1,4-mannosyl-glycoprotein 4-β-N-acetylglucosaminyltransferase (GlcNAc-T III) is considered as a bisecting structure that is usually not considered as an antenna because it cannot be further extended by the proper enzymes. Such antibody and antibody mimetic variants can have reduced fucosylation and/or improved ADCC antibody effector function.. In some embodiments, antibody or antibody mimetic variants can include at least one galactose residue in the oligosaccharide attached to the Fc region. Such antibody and antibody mimetic variants can have improved antibody-mediated complement function.. In some embodiments, the Fc region of an antibody or antibody mimetic can include modifications, e.g., substitutions, in one or more amino acids, thereby generating an Fc region variant. In some embodiments, the Fc region variant can include a human Fc region sequence (e.g., a human IgG1, IgG2, IgG3 or IgG4 Fc region). In some embodiments, an antibody or antibody mimetic variant includes an Fc region with one or more amino acid substitutions can have improved ADCC, e.g., substitutions at positions 298, 333, and/or 334 of the Fc region of SEQ ID NO:698 (Heavy chain of E27 Anti-IgE antibody). In some embodiments, an antibody or antibody mimetic variant includes a modification in the Fc region that results in altered (i.e., either improved or diminished) C1q binding and/or Complement Dependent Cytotoxicity (CDC).). In some embodiments, an antibody or antibody mimetic disclosed herein can be derivatized with non-proteinaceous moieties that are known in the art. In some embodiments, the non-proteinaceous moieties suitable for derivatization of the antibody or antibody mimetic include but are not limited to water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), and/or polyvinyl alcohol, and combinations thereof. The polymer can be of any molecular weight, and can be branched or unbranched. The number of polymers attached to the antibody can vary, and if more than one polymer is attached, they can be the same or different molecules. In general, the number and/or type of polymers used for derivatization can be determined based on considerations including, but not limited to, the particular properties or functions of the antibody or antibody mimetic to be improved, and whether the antibody or antibody mimetic derivative will be used in a therapy under defined conditions. The antibody- or antibody mimetic-polymer conjugates can be made using any suitable technique for derivatizing antibodies with polymers. In some embodiments, the antibody- and antibody mimetic-polymer conjugates as disclosed herein include species wherein a polymer is covalently attached to a specific site or specific sites on the parental antibody, i.e., polymer attachment is targeted to a particular region or a particular amino acid residue or residues in the parental antibody or antibody mimetic. Site specific conjugation of polymers can be performed by attachment to cysteine residues in the parental antibody or antibody mimetic. In such embodiments, the coupling chemistry can, for example, utilize the free sulfhydryl group of a cysteine residue not in a disulfide bridge in the parental antibody. The polymer can be activated with any functional group capable of reacting specifically with the free sulfhydryl or thiol group(s) on the parental antibody, such as maleimide, sulfhydryl, thiol, triflate, tesylate, aziridine, exirane, and 5-pyridyl functional groups. The polymer can be coupled to the parental antibody using any protocol suitable for the chemistry of the coupling system selected.. In some embodiments, one or more cysteine residues naturally present in the parental antibody or antibody mimetic are used as attachment sites for polymer conjugation. In another embodiment, one or more cysteine residues are engineered into a selected site or sites in the parental antibody or antibody mimetic for the purpose of providing a specific attachment site or sites for polymer. In some embodiments, antibody fragments, such as Fab, can be derivatized to form antibody fragment-polymer conjugates, and the polymer is attached to one or more cysteine residue in the light or heavy chain of the fragment that would ordinarily form the inter-chain disulfide bond linking the light and heavy chains.

A skilled artisan will understand that any of the embodiments disclosed herein that include antibodies and antibody mimetics can instead include the antibody variants and antibody mimetic variants, respectively, disclosed in this section.

### Antibody and Antibody Mimetic Effector Functions

The domains of the antibodies and/or antibody mimetics bound by the anti-idiotype polypeptides can control or modulate the downstream effects the anti-idiotype polypeptides have upon binding to their target antibodies or target antibody mimetics. Fc domains of an antibody can change the downstream effects of the antibody binding to an antigen. Thus, illustrative methods provided herein, that include contacting a cell expressing an anti-idiotype polypeptide with a target antibody comprising the idiotype, or delivering an antibody to a subject to effect such contacting, can utilize antibodies whose Fc domains are selected to provide a particular function. Not to be limited by theory, the Fc domain of the antibody can also affect the function of the antibody binding to the anti-idiotype polypeptide, also referred to herein as the antibody effector function. In some embodiments, the target antibody or antibody mimetic includes an Fc domain that is capable of cross-linking with the Fc receptors on an effector cell to initiate antibody-dependent cellular cytotoxicity (ADCC) leading to death of the cells that are bind the target antibody or antibody mimetic. In some embodiments, the effector cell may be one or more of NK cells, monocytes, macrophages, and granulocytes. The Fc domain variants include five isotypes, IgM, IgD, IgG, IgA, and IgE, each with unique structural features that impact the antibody function. The IgG isotype is further divided into four subclasses, i.e., IgG1, IgG2, IgG3, and IgG4, and the IgA isotype is further divided into two subclasses, i.e., IgA1 and IgA2. In some embodiments, the antibody recognized by an anti-idiotype polypeptide includes an Fc domain from IgM, IgD, IgG, IgA, or IgE. In some embodiments, the antibody recognized by an anti-idiotype polypeptide includes an Fc domain from IgM, IgD, IgG1, IgG2, IgG3, IgG4, IgA1, IgA2, or IgE. In some embodiments, the antibody recognized by an anti-idiotype polypeptide includes an Fc domain from IgG1, IgG2, IgG3, or IgG4. In some embodiments, the antibody recognized by an anti-idiotype polypeptide includes an Fc domain from IgA1 or IgA2. In some embodiments, the Fc domain of the antibody recognized by an anti-idiotype polypeptide is or is derived from the Fc domain of an antibody from any animal. In some embodiments, the Fc domain of the antibody recognized by an anti-idiotype polypeptide is or is derived from a rat, mouse, or in illustrative embodiments a human Fc domain and retains at least 85%, 90%, 95%, or 99% sequence identity. In some embodiments, the Fc domain of the antibody recognized by an anti-idiotype polypeptide can be chimeric IgG1, human IgG1, human IgG2, human IgG4, human IgM, humanized IgG1, humanized IgG2, humanized IgG2/4, humanized IgG4, mouse IgG1, or mouse IgG2a.

In some embodiments, the anti-idiotype polypeptide induces, promotes, or activates antibody effector functions upon antibody or antibody mimetic binding to the anti-idiotype extracellular recognition domain of the anti-idiotype polypeptide expressed on the surface of the cell. In some embodiments, binding of the antibody or antibody mimetic to the anti-idiotype polypeptide induces, promotes, or activates one or more of antibody-mediated complement activation, antibody-dependent cellular cytotoxicity (ADCC), complement-dependent cytotoxicity (CDC), antibody-dependent cellular phagocytosis, antibody-dependent enhancement of diseases, and opsonization. In some embodiments, binding of the antibody or antibody mimetic to the anti-idiotype polypeptide does not induce, promote, or activate ADCC. In some embodiments, the antibody includes an Fc domain derived from IgM, IgG, or IgA and binding of the anti-idiotype polypeptide to the antibody induces, promotes, or activates one or more of macrophage opsonophagocytosis, oxidative burst or release of cytokines, and antimicrobial peptides. In some embodiments, the antibody includes an Fc domain derived from IgM, IgG, or IgA and binding of the anti-idiotype polypeptide to the antibody induces, promotes, or activates one or more of antigen uptake, DC maturation, and antigen presentation. In some embodiments, the antibody includes an Fc domain derived from IgM, IgG, or IgA and binding of the anti-idiotype polypeptide to the antibody induces, promotes, or activates antigen capture on follicular dendritic cells for presentation to B cells. In some embodiments, the antibody includes an Fc domain derived from IgD, IgG, or IgE and binding of the anti-idiotype polypeptide to the antibody induces, promotes, or activates granulocyte degranulation and release of one or more of vasoactive mediators, chemoattractants and T_{H}2-type cytokines. In some embodiments, the antibody includes an Fc domain derived from IgM or IgG and binding of the anti-idiotype polypeptide to the antibody induces, promotes, or activates complement-mediated production of chemoattractants, cytotoxicity, and opsonophagocytosis. In some embodiments, the antibody includes an Fc domain derived from IgM or IgG and binding of the anti-idiotype polypeptide to the antibody induces, promotes, or activates one or more of neutrophil activation, opsonophagocytosis, oxidative burst, and induction of neutrophil extracellular traps. In some embodiments, the antibody includes an Fc domain derived from IgG and binding of the anti-idiotype polypeptide to the antibody induces, promotes, or activates one or more of NK cell degranulation and cytotoxicity.

### Anti-Idiotype Compositions

In one aspect, provided herein is an anti-idiotype polypeptide that includes an extracellular recognition domain and a membrane association domain, wherein the extracellular recognition domain comprises a domain that recognizes an idiotype of a target antibody or a target antibody mimetic.

Furthermore, provided herein in another aspect is a polynucleotide encoding the anti-idiotype polypeptide. For example, in one aspect provided herein is a polynucleotide that includes one or more transcriptional units, wherein each of the one or more transcriptional units is operatively linked to a promoter wherein the one or more transcriptional units comprise:
a) nucleic acids a polynucleotide sequence encoding one or more inhibitory RNA molecules and/or a first engineered signaling polypeptide, and
b) nucleic acids a polynucleotide sequence encoding an anti-idiotype polypeptide comprising an extracellular recognition domain and a membrane association domain, wherein the extracellular recognition domain comprises a domain that recognizes an idiotype of a target antibody or antibody mimetic.

In some embodiments, the anti-idiotype polypeptide further includes one or more of a stalk domain, an intracellular domain, and/or a linker. The anti-idiotype polypeptide can also be part a fusion polypeptide, as disclosed in more detail below. In such embodiments, the membrane association domain can be part of the polypeptide to which the anti-idiotype polypeptide is fused.

In some embodiments, the anti-idiotype polypeptide can be at least 10, 15, 20, 25, 30, 35, 40, 45, 50, 75, 100, 125, 150, 175, 200, 225, 250, 275, 300, 350, 400, 450, 500, 600, 700, 800, 900, or 1000 amino acids in length. In some embodiments, the anti-idiotype polypeptide can be between 10 and 1000, 10 and 900, 10 and 800, 10 and 700, 10 and 600, 10 and 500, 10 and 400, 10 and 300, 10 and 200, 10 and 100, 10 and 50, 25 and 1000, 25 and 900, 25 and 800, 25 and 700, 25 and 600, 25 and 500, 25 and 400, 25 and 300, 25 and 200, 25 and 100, 25 and 50, 50 and 1000, 50 and 900, 50 and 800, 50 and 700, 50 and 600, 50 and 500, 50 and 400, 50 and 300, 50 and 200, 50 and 100, 100 and 1000, 100 and 900, 100 and 800, 100 and 700, 100 and 600, 100 and 500, 100 and 400, 100 and 300, or 100 and 200 amino acids in length.

### Anti-Idiotype Extracellular Recognition Domain

The anti-idiotype extracellular recognition domain of an anti-idiotype polypeptide is typically all or more typically part of an anti-idiotype antibody or anti-idiotype antibody mimetic. The extracellular recognition domain of an anti-idiotype polypeptide is capable of, has a structure for, is designed to, is selected for, is effective for, and/or is adapted for, recognizing, binding to, or otherwise interacting with the idiotype of a target antibody or the idiotype of a target antibody mimetic. Thus, a recognition domain of an anti-idiotype antibody or antibody mimetic, and the target antibody or antibody mimetic that has the idiotype to which it binds, are specific binding pair members.

In some embodiments, an extracellular recognition domain of an anti-idiotype polypeptide recognizes the idiotype of any antibody or antibody mimetic known in the art, which can be, for non-limiting example, a clinical antibody or clinical antibody mimetic, as discussed in further detail herein. In some embodiments, the extracellular recognition domain of an anti-idiotype polypeptide herein includes an idiotype-binding variable region of an anti-idiotype antibody or an idiotype-binding region of an anti-idiotype antibody mimetic. Such variable region can include a framework that is derived from a human framework region. Such anti-idiotype antibody or antibody mimetic can be any of antibody fragment or any type of antibody mimetic provided herein. As non-limiting examples, the extracellular recognition domain can be an antibody such as a full-length antibody, a single-chain antibody, a Fab fragment, a Fab′ fragment, a (Fab′)2 fragment, a Fv fragment, an scFv, a divalent single-chain antibody, a diabody, a chimeric antibody, or a disbud. In some embodiments, the extracellular recognition domain is a single chain Fv (scFv). In some embodiments, the heavy chain is positioned N-terminal of the light chain in the engineered signaling polypeptide. In other embodiments, the light chain is positioned N-terminal of the heavy chain in the engineered signaling polypeptide. In any of the disclosed embodiments, the heavy and light chains can be separated by a linker as discussed in more detail herein. In any of the disclosed embodiments, the heavy or light chain can be at the N-terminus of the engineered signaling polypeptide and is typically C-terminal of another domain, such as a signal sequence or peptide.

In some embodiments, the extracellular recognition domain can include one or more domains from or derived from a mouse antibody, a rat antibody, a rabbit antibody, a goat antibody, a chicken antibody, a sheep antibody, a cow antibody, or a llama antibody, or in illustrative embodiments, a human antibody. Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use as extracellular recognition domains of anti-idiotype polypeptides provided herein, and methods using the same. In some instances, T cell receptor (TCR) based recognition domains can be the extracellular recognition domains. In some embodiments, the extracellular recognition domain can be bispecific or include domains from a bispecific antibody. In some embodiments, the extracellular recognition domain can be multispecific or include domains from a multispecific antibody. Multispecific antibodies have binding specificities for at least two different sites. In certain embodiments, one of the binding specificities is for one target antibody or antibody mimetic and the other is for another target antibody or antibody mimetic. In certain embodiments, bispecific antibodies may bind to two different epitopes of a target antibody or antibody mimetic. In some embodiments, the extracellular recognition domain can include a recognition domain of an anti-idiotype antibody or antibody mimetic, and also include an ASTR of any of the CARs disclosed herein.

In some embodiments, the extracellular recognition domain can be at least 10, 15, 20, 25, 30, 35, 40, 45, 50, 75, 100, 125, 150, 175, 200, 225, 250, 275, or 300 amino acids in length. In some embodiments, the extracellular recognition domain can be between 10 and 500, 10 and 400, 10 and 300, 10 and 200, 10 and 100, 10 and 50, 10 and 30, 20 and 500, 20 and 400, 20 and 300, 20 and 200, 20 and 100, 20 and 50, 20 and 30, 50 and 500, 50 and 400, 50 and 300, 50 and 200, 50 and 100, 100 and 500, 100 and 400, 100 and 300, 100 and 200, 125 and 275, 150 and 275, or 150 and 250 amino acids in length.

Methods for making anti-idiotype extracellular recognition domains provided herein, can utilize known methods for making anti-idiotype antibodies and antibody mimetics, applying a strategy, for example that is provided in the Examples herein for an exemplary phage display strategy. Such methods can include makine a library of antibodies or antibody mimetics using virtually any method known in the art for creating such library, and screening the library for binding to the idiotype of a target antibody. The library can be constructed by varying residues of an existing anti-idiotype antibody, or can involve a general antibody or antibody mimetic library that does not start with a selected prior anti-idiotype antibodies. Once the library is designed and constructed, the translated antibody or antibody mimetic protein scaffolds are screened for isolation of mutants with the desired properties. The most commonly employed display systems include phage display, ribosome display, mRNA display, yeast display, and bacterial cell-surface display (Lipovsek and Plückthun, J Immunol Methods. 2004 Jul;290(1-2):51-67). A skilled artisan will understand how to use this and similar systems to identify appropriate anti-idiotype antbodies and antibody mimetics, typically using all or a portion of the target antibody as bate, which can be present on a solid support such as a bead during the screening. Screening can occur over several rounds, where conditions can be varied to improve isolated library members for one or more characteristics, including affinity for the idiotype. Other antibodies or antibody mimetics that have similar constant regions as a target antibody, but a different idiotype can be used to subtract out any library members that bind the target antibody non-specifically. Examples of using phage display to screen for, identify and isolate anti-idiotype antibodies are provided in the Examples herein.

In some embodiments, binding of the anti-id ERD of the anti-idiotype polypeptide to its target antibody or antibody mimetic prevents and/or blocks binding of the target antibody or antibody mimetic to the cognate antigen of the target antibody or antibody mimetic. In some embodiments, the extracellular recognition domain of the anti-idiotype polypeptide is capable of, adapted for, and/or configured to prevent and/or block binding of the target antibody or antibody mimetic to the cognate antigen of the target antibody or antibody mimetic when the anti-idiotype polypeptide is bound to the target antibody or antibody mimetic. In illustrative embodiments, the target antibody is cetuximab and binding of the extracellular recognition domain of the anti-idiotype polypeptide to cetuximab prevents and/or blocks binding of cetuximab to Epidermal Growth Factor Receptor (EGFR). In some embodiments, the extracellular recognition domain of the anti-idiotype polypeptide is capable of, adapted for, and/or configured to prevent and/or block binding of cetuximab to EGFR when the anti-idiotype polypeptide is bound to cetuximab. In some embodiments, binding of the anti-idiotype polypeptide to the target antibody does not block or prevent binding between the target antibody and its cognate antigen. In illustrative embodiments, binding of the anti-idiotype polypeptide to the target antibody blocks or prevents binding between the target antibody and its cognate antigen. In some embodiments, the extracellular recognition domain recognizes the antigen-binding site of the target antibody or the antibody mimetic. In some embodiments, the extracellular recognition domain recognizes the variable region but does not recognize the antigen-binding site of the target antibody or the antibody mimetic. In some embodiments, the extracellular recognition domain recognizes a portion of the variable region that is not a part of the antigen-binding site of the target antibody or the antibody mimetic. In some embodiments where the extracellular recognition domain recognizes the antigen-binding site of the target antibody or the antibody mimetic, binding of the extracellular recognition domain to the target antibody or antibody mimetic prevents and/or blocks binding of the target antibody or antibody mimetic to the cognate antigen of the target antibody or antibody mimetic. The antigen-binding site can include the residues important for the target antibody or antibody mimetic to recognize its cognate antigen.

In some embodiments, the anti-id ERD recognizes (e.g., is capable of binding to) cetuximab and comprises any of the sequences provided in this paragraph. In some embodiments, the anti-id ERD can be encoded by a polynucleotide with at least 50%, 60%, 70%, 75%, 80%, 85%, 90%, 95%, 96%, 97%, 98%, 99% or 100% sequence identity to a stretch of at least 10, 15, 20, or all the polynucleotides of one or more of SEQ ID NOs:376-436. In any of the embodiments disclosed herein that include an anti-id ERD, the anti-id ERD can include a polypeptide sequence with at least 50%, 60%, 70%, 75%, 80%, 85%, 90%, 95%, 96%, 97%, 98%, 99% or 100% sequence identity to a stretch of at least 10, 15, 20, or all the amino acids of one or more of SEQ ID NOs:437-497. In some embodiments, the extracellular recognition domain can include a polypeptide sequence with at least 50%, 60%, 70%, 75%, 80%, 85%, 90%, 95%, 96%, 97%, 98%, 99% or 100% sequence identity to a stretch of at least 10, 15, 20, or all of the amino acids of one or more of SEQ ID NOs:498-599. In some embodiments, the extracellular recognition domain can include a polypeptide sequence with at least 50%, 60%, 70%, 75%, 80%, 85%, 90%, 95%, 96%, 97%, 98%, 99% or 100% sequence identity to a stretch of at least 10, 15, 20, or all of the amino acids of one or more of SEQ ID NOs:600-672.

In some embodiments, the anti-id ERD recognizes (e.g., is capable of binding to) cetuximab and comprises any of the sequences provided in this paragraph. In some embodiments, the HCDR1, HCDR2, and HCDR3 of an extracellular recognition domain can include any of the following combinations of SEQ ID NOs:498, 528, and 560; 499, 529, and 561; 500, 530, and 562; 501, 531, and 563; 502, 532, and 564; 503, 533, and 565; 504, 534, and 566; 505, 535, and 567; 506, 536, and 568; 507, 537, and 569; 508, 538, and 570; 509, 539, and 571; 510, 540, and 572; 511, 541, and 573; 511, 542, and 574; 501, 543, and 575; 512, 544, and 576; 513, 545, and 577; 501, 543, and 578; 514, 546, and 579; 513, 545, and 580; 515, 547, and 581; 516, 548, and 582; 517, 549, and 583; 517, 549, and 584; 518, 550, and 585; 519, 551, and 586; 520, 552, and 587; 521, 553, and 588; 522, 554, and 589; 523, 555, and 590; 513, 545, and 591; 517, 549, and 592; 524, 556, and 593; 513, 545, and 594; 511, 542, and 595; 525, 557, and 596; 526, 558, and 597; 527, 559, and 598; or 503, 533, and 599, respectively, or polypeptide sequences at least 50%, 60%, 70%, 75%, 80%, 85%, 90%, 95%, 96%, 97%, 98%, 99% or 100% sequence identity to a stretch of at least 10, 15, 20, or all of the amino acids of any of the preceding SEQ ID NOs.

In some embodiments, the anti-id ERD recognizes (e.g., is capable of binding to) cetuximab and comprises any of the sequences provided in this paragraph. In some embodiments, the LCDR1, LCDR2, and LCDR3 of an extracellular recognition domain can include any of the following combinations of SEQ ID NOs:600, 625, and 634; 601, 625, and 635; 600, 625, and 636; 602, 625, and 637; 603, 626, and 638; 603, 626, and 639; 604, 630, and 640; 605, 628, and 641; 606, 625, and 642; 603, 626, and 643; 607, 632, and 644; 608, 625, and 645; 609, 625, and 646; 610, 630, and 647; 604, 630, and 648; 611, 625, and 649; 606, 625, and 650; 612, 625, and 649; 607, 632, and 651; 600, 625, and 652; 600, 625, and 653; 600, 625, and 654; 613, 632, and 655; 614, 625, and 656; 604, 630, and 657; 615, 630, and 658; 616, 625, and 659; 606, 625, and 660; 617, 625, and 661; 604, 630, and 662; 618, 632, and 663; 604, 630, and 664; 619, 630, and 665; 620, 630, and 666; 621, 631, and 667; 606, 627, and 642; 622, 629, and 668; 623, 629, and 669; 600, 625, and 670; 600, 625, and 671; 600, 625, and 672; or 624, 633, and 659, respectively, or polypeptide sequences at least 50%, 60%, 70%, 75%, 80%, 85%, 90%, 95%, 96%, 97%, 98%, 99% or 100% sequence identity to a stretch of at least 10, 15, 20, or all of the amino acids of any of the preceding SEQ ID NOs.

In some embodiments, the anti-id ERD recognizes (e.g., is capable of binding to) cetuximab and comprises any of the sequences provided in this paragraph. In some embodiments, the HCDR1, HCDR2, HCDR3, LCDR1, LCDR2, and LCDR3 of an extracellular recognition domain can include any of the following combinations of SEQ ID NOs: 498, 528, 560, 600, 625, and 634; 499, 529, 561, 601, 625, and 635; 500, 530, 562, 600, 625, and 636; 501, 531, 563, 602, 625, and 637; 502, 532, 564, 603, 626, and 638; 503, 533, 565, 600, 625, and 634; 504, 534, 566, 603, 626, and 639; 504, 534, 566, 604, 630, and 640; 505, 535, 567, 605, 628, and 641; 506, 536, 568, 606, 625, and 642; 507, 537, 569, 603, 626, and 643; 508, 538, 570, 607, 632, and 644; 509, 539, 571, 608, 625, and 645; 510, 540, 572, 609, 625, and 646; 511, 541, 573, 610, 630, and 647; 511, 542, 574, 604, 630, and 648; 501, 543, 575, 611, 625, and 649; 501, 543, 575, 606, 625, and 650; 501, 543, 575, 612, 625, and 649; 512, 544, 576, 607, 632, and 651; 513, 545, 577, 600, 625, and 652; 501, 543, 578, 600, 625, and 653; 514, 546, 579, 600, 625, and 654; 513, 545, 580, 613, 632, and 655; 513, 545, 580, 606, 625, and 650; 515, 547, 581, 614, 625, and 656; 516, 548, 582, 600, 625, and 634; 517, 549, 583, 604, 630, and 657; 517, 549, 584, 615, 630, and 658; 518, 550, 585, 616, 625, and 659; 519, 551, 586, 606, 625, and 660; 519, 551, 586, 617, 625, and 661; 520, 552, 587, 604, 630, and 662; 521, 553, 588, 618, 632, and 663; 522, 554, 589, 604, 630, and 664; 523, 555, 590, 619, 630, and 665; 513, 545, 591, 620, 630, and 666; 517, 549, 592, 621, 631, and 667; 524, 556, 593, 606, 627, and 642; 513, 545, 594, 622, 629, and 668; 511, 542, 595, 600, 625, and 634; 525, 557, 596, 623, 629, and 669; 500, 530, 562, 600, 625, and 670; 526, 558, 597, 600, 625, and 671; 504, 534, 566, 600, 625, and 672; 527, 559, 598, 600, 625, and 634; 503, 533, 599, 600, 625, and 634; or 518, 550, 585, 624, 633, and 659, respectively, or polypeptide sequences at least 50%, 60%, 70%, 75%, 80%, 85%, 90%, 95%, 96%, 97%, 98%, 99% or 100% sequence identity to a stretch of at least 10, 15, 20, or all of the amino acids of any of the preceding SEQ ID NOs.

In some embodiments, the extracellular recognition domain can be from or derived from an anti-idiotype antibody mimetic. In some embodiments the anti-idiotype antibody mimetic can be an afflilin, an affimer, an affitin, an alphabody, an alphamab, an anticalin, a peptide aptamer, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a nanofitin, a leucine-rich repeat domain, a lipocalin domain, a mAb 2 or Fcab™, a nanobody, a nanoCLAMP, an OBody, a Pronectin, a single-chain TCR, a tetratricopeptide repeat domain, VHH, or a V-like domain. Not to be limited by theory, antibody mimetics may provide superior properties over antibodies including, but not limited to, superior solubility, tissue penetration, stability towards heat and enzymes (e.g., resistance to enzymatic degradation), and lower production costs.

In some embodiments, the extracellular recognition domain of the anti-idiotype polypeptide includes an affimer that is capable of binding to a target antibody. The average size of the affimer can be in the range of 12-14 kDa. Not to be limited by theory, the affimers capable of binding to a specific target is designed by selecting an appropriate scaffold that is suitable for incorporation of recognition domains. The scaffold is a three-dimensional protein structure that is suitable for mutations and insertions with enough flexibility in its primary structure that the introduced modifications do not compromise its secondary structure and overall stability. The scaffolds are typically small, thermostable, single-domain proteins without any disulfide bonds or glycosylation. Affimers can be made from two scaffolds, Adhiron scaffolds and human stefin A scaffolds. The stefin A scaffold is engineered from human stefin A protein, whereas the Adhiron scaffold is synthetic, originally based on the sequence of cystatin. After the selection of the scaffold, the library is designed by employing in silico methods and is constructed via molecular biology protocols. Once the possible candidates for mutagenesis are identified, mutant constructs and libraries of the antibody mimetics can be generated at the DNA level by employing either site directed or random mutagenesis strategies. Once the library is designed and constructed, the translated protein scaffolds are screened for isolation of mutants with the desired properties. The most commonly employed display systems include phage display, ribosome display, mRNA display, yeast display, and bacterial cell-surface display. A skilled artisan will understand how to use this and similar systems to identify appropriate anti-idiotype affimers.

In some embodiments, the extracellular recognition domain of the anti-idiotype polypeptide includes a DARPin that is capable of binding to a target antibody. DARPins are made from tightly packed repeats of 33 amino acid residues. Each repeat forms a structural unit consisting of a β-turn followed by two antiparallel α-helices. DARPins are small proteins having a molecular weight in the range of 14-18 kDa that are extremely thermostable and resistant to proteases and denaturing agents. DARPins usually have a scaffold that is a constant region, and have variable sites in which amino acid substitutions do not alter the protein conformation. The process of designing scaffolds involves the design of a library of protein variants by random site-specific mutagenesis; and selection of molecules using techniques like yeast display, phage display, and ribosome display. DARPins can recognize targets with high specificities and affinities, surpassing that of the antibodies. A skilled artisan will understand how to use this and similar systems to identify appropriate anti-idiotype DARPins.

In some embodiments, the extracellular recognition domain of the anti-idiotype polypeptide includes a nanobody that is capable of binding to a target antibody. A nanobody is an antigen-binding fragment, with a size of approximately 12-15 kDa. Not to be limited by theory, the nanobody includes the antigen-binding capacity of the original heavy chain antibodies, evolved to be fully functional in the absence of a light chain. Nanobodies consist of three antigenic complementary determining regions (CDRs) and four frame regions (FRs). The CDRs are the binding regions of nanobody to the antigen or a target antibody. To generate nanobodies, a library is prepared from a sample that includes camelids. Phage display can be used to screen and enrich nanobody-phage with specific binding ability from the nanobody library. Functional verification and confirmation of the nanobody expression can also be performed to select the optimal candidate. A skilled artisan will understand how to use this and similar systems to identify appropriate anti-idiotype nanobodies.

### Stalk Domain

In some embodiments, the anti-idiotype polypeptide includes a stalk which is located in the portion of the anti-idiotype polypeptide lying outside the cell and interposed between the extracellular recognition domain and the membrane association domain. In some embodiments, the stalk has at least 85, 90, 95, 96, 97, 98, 99, or 100% identity to a wild-type CD8 stalk region (TTTPAPRPPTPAPTIASQPLSLRPEACRPAAGGAVHTRGLDFA (SEQ ID NO:2), has at least 85, 90, 95, 96, 97, 98, 99, or 100% identity to a wild-type CD28 stalk region (FCKIEVMYPPPYLDNEKSNGTIIHVKGKHLCPSPLFPGPSKP (SEQ ID NO:3)), or has at least 85, 90, 95, 96, 97, 98, 99, or 100% identity to a wild-type immunoglobulin heavy chain stalk region. In an anti-idiotype polypeptide, the stalk employed allows the extracellular recognition domain, and typically the entire anti-idiotype polypeptide, to retain increased binding to a target antibody or antibody mimetic. In some embodiments, the stalk domain can be heavy chain Fc domain.

The stalk region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

In some embodiments, the stalk of an anti-idiotype polypeptide includes at least one cysteine. For example, in some embodiments, the stalk can include the sequence Cys-Pro-Pro-Cys (SEQ ID NO:4). If present, a cysteine in the stalk of a first anti-idiotype polypeptide can be available to form a disulfide bond with a stalk in a second anti-idiotype polypeptide.

Stalks can include immunoglobulin hinge region amino acid sequences that are known in the art; see, e.g., Tan et al. (1990) Proc. Natl. Acad. Sci. USA 87:162; and Huck et al. (1986) Nucl. Acids Res. 14:1779. As non-limiting examples, an immunoglobulin hinge region can include a domain with at least 50, 60, 70, 75, 80, 85, 90, 95, 96, 97, 98, 99 or 100% sequence identity to a stretch of at least 10, 15, 20, or all of the amino acids of any of the following amino acid sequences: DKTHT (SEQ ID NO:5); CPPC (SEQ ID NO:4); CPEPKSCDTPPPCPR (SEQ ID NO:6) (see, e.g., Glaser et al. (2005) J. Biol. Chem. 280:41494); ELKTPLGDTTHT (SEQ ID NO:7); KSCDKTHTCP (SEQ ID NO:8); KCCVDCP (SEQ ID NO:9); KYGPPCP (SEQ ID NO: 10); EPKSCDKTHTCPPCP (SEQ ID NO:11) (human IgG1 hinge); ERKCCVECPPCP (SEQ ID NO:12) (human IgG2 hinge); ELKTPLGDTTHTCPRCP (SEQ ID NO:13) (human IgG3 hinge); SPNMVPHAHHAQ (SEQ ID NO:14) (human IgG4 hinge); and the like. The stalk can include a hinge region with an amino acid sequence of a human IgG1, IgG2, IgG3, or IgG4, hinge region. The stalk can include one or more amino acid substitutions and/or insertions and/or deletions compared to a wild-type (naturally-occurring) hinge region. For example, His229 of human IgG 1 hinge can be substituted with Tyr, so that the stalk includes the sequence EPKSCDKTYTCPPCP (SEQ ID NO:15), (see, e.g., Yan et al. (2012) J. Biol. Chem. 287:5891). The stalk can include an amino acid sequence derived from human CD8; e.g., the stalk can include the amino acid sequence: TTTPAPRPPTPAPTIASQPLSLRPEACRPAAGGAVHTRGLDFACD (SEQ ID NO:16), or a variant thereof.

In some embodiments, the stalk domain of an anti-idiotype polypeptide can include any of the dimerizing moieties disclosed herein. In any aspects or embodiments wherein the stalk domain of an anti-idiotype polypeptide includes a dimerizing motif, the dimerizing motif can be selected from the group consisting of: a leucine zipper motif-containing polypeptide, CD69, CD71, CD72, CD96, Cd105, Cd161, Cd162, Cd249, CD271, and Cd324, as well as mutants and/or active fragments thereof that retain the ability to dimerize.

### Membrane Association Domain

The membrane association domain of an anti-idiotype polypeptide attaches the extracellular recognition domain of the anti-idiotype polypeptide to a cell membrane. In some embodiments, the membrane association is a transmembrane domain. In some embodiments, the transmembrane domain is a heterologous transmembrane domain. In other embodiments, the transmembrane domain is an endogenous transmembrane domain. In some embodiments, the transmembrane domain is from or derived from an antibody. In illustrative embodiments, the transmembrane domain is from or derived from IgD. In some embodiments, including embodiments where the transmembrane domain is from or derived from IgD, the polynucleotide, vector, or cell can further include nucleic acids encoding IgA and IgB. CA

In some embodiments, the transmembrane domain is from or derived from BAFFR, C3Z, CEACAM1, CD2, CD3A, CD3B, CD3D, CD3E, CD3G, CD3Z, CD4, CD5, CD7, CD8A, CD8B, CD9, CD11A, CD11B, CD11C, CD11D, CD27, CD16, CD18, CD19, CD22, CD28, CD29, CD33, CD37, CD40, CD45, CD49A, CD49D, CD49F, CD64, CD79A, CD79B, CD80, CD84, CD86, CD96 (Tactile), CD100 (SEMA4D), CD103, C134, CD137, CD154, CD160 (BY55), CD162 (SELPLG), CD226 (DNAM1), CD229 (Ly9), CD247, CRLF2, CRTAM, CSF2RA, CSF2RB, CSF3R, EPOR, FCER1G, FCGR2C, FCGRA2, GHR, HVEM (LIGHTR), IA4, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL7RA Ins PPCL, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA1, ITGA4, ITGA6, ITGAD, ITGAE, ITGAL, ITGAM, ITGAX, ITGB1, ITGB2, ITGB7, KIRDS2, LEPR, LFA-1 (CD11a, CD18), LIFR, LTBR, MPL, NKp80 (KLRF1), OSMR, PAG/Cbp, PRLR, PSGL1, SLAM (SLAMF1, CD150, IPO-3), SLAMF4 (CD244, 2B4), SLAMF6 (NTB-A, Ly108), SLAMF7, SLAMF8 (BLAME), TNFR2, TNFRSF4, TNFRSF8, TNFRSF9, TNFRSF14, TNFRSF18, VLA1, or VLA-6, or functional mutants and/or fragments thereof. In some embodiments, the transmembrane domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of the amino acids of one or more of the following: CD8 alpha TM (SEQ ID NO: 17); CD8 beta TM (SEQ ID NO:18); CD4 stalk (SEQ ID NO:19); CD3Z TM (SEQ ID NO:20); CD28 TM (SEQ ID NO:21); CD134 (OX40) TM: (SEQ ID NO:22); CD7 TM (SEQ ID NO:23); CD8 stalk and TM (SEQ ID NO:24); CD28 stalk and TM (SEQ ID NO:25); the hPDGFRb hinge and TM of SEQ ID NO:676 (this sequence also includes an 8 aa intracellular domain); the CD28 hinge and TM of SEQ ID NO:677 (this sequence also includes a 9 aa intracellular domain); the CD80 hinge and TM domain of SEQ ID NO:678 (this sequence also includes an intracellular domain); or the CD28 hinge and TM of SEQ ID NO:679 (this sequence also includes an intracellular domain).

In some embodiments, the transmembrane domain of an anti-idiotype polypeptide can include any of the dimerizing moieties disclosed herein. In any aspects or embodiments wherein the transmembrane domain of an anti-idiotype polypeptide includes a dimerizing motif, the dimerizing motif can be selected from the group consisting of: a leucine zipper motif-containing polypeptide, CD69, CD71, CD72, CD96, Cd105, Cd161, Cd162, Cd249, CD271, and Cd324, as well as mutants and/or active fragments thereof that retain the ability to dimerize.

In some embodiments, the membrane association domain is glycosylphosphatidylinositol (GPI). In some embodiments, the anti-idiotype polypeptide is fused to a lymphoproliferative element, CAR, and/or recombinant TCR, and the membrane association domain can be the transmembrane domain of the lymphoproliferative element, the CAR, and/or the recombinant TCR.

### Intracellular Domain

The intracellular domain of an anti-idiotype polypeptide, if present, is typically all or, more typically, part of one or more intracellular domains known in the art or disclosed herein. The intracellular domain of an anti-idiotype polypeptide can perform various functions, including both non-signaling functions, for example, to anchor the anti-idiotype polypeptide, and signaling functions, for example, to activate proliferative, survival, and/or cell death signaling or modulate transcriptional activity. Thus, in some embodiments, an anti-idiotype polypeptide includes an intracellular domain. In some embodiments, for example, when one or more of the anti-idiotype polypeptide domains are part of a fusion polypeptide, the intracellular domain of the fusion polypeptide can be the anti-idiotype polypeptide intracellular domain. In other embodiments, the intracellular domain of the fusion polypeptide can be the intracellular domain of the polypeptide to which the anti-idiotype polypeptide is fused, for example, the intracellular domain of a lymphoproliferative element, a CAR, and/or a recombinant TCR. The intracellular domains that can be used include any of the intracellular domains of lymphoproliferative elements, CARs, and recombinant TCRs are disclosed elsewhere herein. Thus, in some embodiments, the intracellular domain of an anti-idiotype polypeptide can include one or more, two or more, or three or more intracellular domains from other polypeptides, for example any of the intracellular domains of a lymphoproliferative element, a CAR, and/or a recombinant TCR disclosed herein. In some embodiments, the intracellular domain can include one or more of any of the intracellular activating domains, modulatory domains, or intracellular signaling domains disclosed elsewhere herein. In some embodiments, the intracellular domain of an anti-idiotype polypeptide can be at least 1, 2, 3, 4, 5, 6, 7, 8, 9, 10, 15, 20, 25, 30 35, 40 45, 50, 75, 100, 125, 150, 175, or 200 amino acids in length. In some embodiments, the intracellular domain of an anti-idiotype polypeptide can be between 1 and 1000, 1 and 900, 1 and 800, 1 and 700, 1 and 600, 1 and 500, 1 and 400, 1 and 300, 1 and 200, 1 and 100, 1 and 50, 1 and 25, 5 and 1000, 5 and 900, 5 and 800, 5 and 700, 5 and 600, 5 and 500, 5 and 400, 5 and 300, 5 and 200, 5 and 100, 5 and 50, 5 and 25, 10 and 1000, 10 and 900, 10 and 800, 10 and 700, 10 and 600, 10 and 500, 10 and 400, 10 and 300, 10 and 200, 10 and 100, 10 and 50, 10 and 25, 25 and 1000, 25 and 900, 25 and 800, 25 and 700, 25 and 600, 25 and 500, 25 and 400, 25 and 300, 25 and 200, 25 and 100, 25 and 50, 50 and 1000, 50 and 900, 50 and 800, 50 and 700, 50 and 600, 50 and 500, 50 and 400, 50 and 300, 50 and 200, 50 and 100, 100 and 1000, 100 and 900, 100 and 800, 100 and 700, 100 and 600, 100 and 500, 100 and 400, 100 and 300, 100 and 200, 200 and 1000, 200 and 900, 200 and 800, 200 and 700, 200 and 600, 200 and 500, 200 and 400, 200 and 300, 300 and 1000, 300 and 900, 300 and 800, 300 and 700, 300 and 600, 300 and 500, or 300 and 400 amino acids in length. In some embodiments, the intracellular domain is an anchor for the anti-idiotype polypeptide. In some embodiments, the intracellular domain has no signaling activity.

In some embodiments, signaling through the intracellular domains of the anti-idiotype polypeptide is inducible, and signaling can be activated by addition of the target antibody or antibody mimetic. Such anti-idiotype polypeptides can be referred to herein as activity modulators, where upon addition of the target antibody or antibody mimetic, two or more anti-idiotype polypeptides can dimerize through binding the same molecule of target antibody or antibody mimetic. This dimerization of the anti-idiotype polypeptides dimerizes the intracellular domains of the two anti-idiotype polypeptides, resulting in activation based if the appropriate intracellular domains are used. In some embodiments, the intracellular domains of an activity modulating anti-idiotype polypeptide can be activated by dimerization and the only dimerizing moiety on the anti-idiotype polypeptide is the extracellular recognition domain. In other embodiments, the intracellular domains of an activity modulating anti-idiotype polypeptide can be activated by dimerization and the anti-idiotype polypeptide includes a separate inducible or constitutive dimerizing moiety on the anti-idiotype polypeptides besides the extracellular recognition domain. In some embodiments, the intracellular domains of an activity modulating anti-idiotype polypeptide can be activated by multimerization of at least three domains (e.g., trimerization) and the only dimerizing moiety on the anti-idiotype polypeptide is the extracellular recognition domain. In other embodiments, the intracellular domains of an activity modulating anti-idiotype polypeptide can be activated by multimerization of at least three domains (e.g., trimerization) and the anti-idiotype polypeptide includes a separate inducible or constitutive dimerizing moiety on the anti-idiotype polypeptides besides the extracellular recognition domain. In illustrative embodiments, the intracellular domains of an activity modulating anti-idiotype polypeptide that are activated by multimerization of at least three domains (e.g., trimerization) include a separate constitutive dimerizing moiety on the anti-idiotype polypeptides besides the extracellular recognition domain. In some embodiments, the multimerization (e.g., dimerization, trimerization, etc.) of an anti-idiotype polypeptide activates proliferative and/or survival signaling. Such embodiments include, for example, fusion polypeptides where the extracellular recognition domain is attached to a lymphoproliferative element, and an anti-idiotype polypeptide with the intracellular domains from a lymphoproliferative element, for example an intracellular signaling domain from a cytokine receptor. In some embodiments, multimerization of an anti-idiotype polypeptide activates cell death signaling. Such embodiments include, for example, an anti-idiotype polypeptide with the intracellular domains from some of the apoptosis-inducing polypeptides disclosed herein. In some embodiments, the target antibody or antibody mimetic recognized by an anti-idiotype polypeptide includes an Fc domain from IgM or IgA, and binding of the target induces multimerization (e.g., trimerization and higher order multimerization). In such embodiments, the target antibody or antibody mimetic can activate intracellular domains that require higher order multimerization (e.g., trimerization). In some embodiments, the anti-idiotype polypeptide recognizes nebacumab, which includes an Fc domain from IgM, and binding of the the anti-idiotype polypeptide to nebacumab induces multimerization, and activation of the intracellular domain of the anti-idiotype polypeptide.

In some embodiments, the intracellular domain can include one or more, two or more, three or more, or all of the domains, motifs, and/or mutations of any of the intracellular domains disclosed herein. In some embodiments, the intracellular domain can include one or more, two or more, three or more, or all of the domains, motifs, and/or mutations of any intracellular domains known to induce proliferation and/or survival of T cells and/or NK cells. In some embodiments, the intracellular domain can activate a Jak pathway, a Stat pathway, a Jak/Stat pathway, a TRAF pathway, a PI3K pathway, and/or a PLC pathway. Appropriate intracellular domains for activating the various pathways are disclosed in the “Lymphoproliferative elements” herein and can be used as intracellular domain of an anti-idiotype polypeptide. In some embodiments, the intracellular domain of an anti-idiotype polypeptide can include all or part of one or more intracellular signaling domains from one or more cytokine receptors. In further embodiments, the one or more cytokine receptors can be selected from CD27, CD40, CRLF2, CSF2RA, CSF2RB, CSF3R, EPOR, GHR, IFNAR1, IFNAR2, IFNGR1, IFNGR2, IFNLR1, IL1R1, IL1RAP, IL1RL1, IL1RL2, IL2R, IL2RA, IL2RB, IL2RG, IL3RA, IL4R, IL5RA, IL6R, IL6ST, IL7R, IL7RA, IL9R, IL10RA, IL10RB, IL11RA, IL12RB1, IL13R, IL13RA1, IL13RA2, IL15R, IL15RA, IL17RA, IL17RB, IL17RC, IL17RE, IL18R1, IL18RAP, IL20RA, IL20RB, IL21R, IL22RA1, IL23R, IL27R, IL27RA, IL31RA, LEPR, LIFR, MPL, OSMR, PRLR, TGFβR, TGF(3 decoy receptor, TNFRSF4, TNFRSF8, TNFRSF9, TNFRSF14, or TNFRSF18.

In some embodiments, the intracellular domain of an anti-idiotype polypeptide includes one or more apoptotic domains known to induce cell death in T cells and/or NK cells. Such intracellular domains are also referred to herein as intracellular apoptotic domains and include domains from apoptosis-inducing polypeptides, referred to herein as apoptotic polypeptides, and functional fragments thereof. The intracellular apoptotic domain of an anti-idiotype polypeptide can include one or more caspase activation and recruitment domains (CARDs), death domains (DDs), death effector domains (DEDs), pyrin domains (PYDs), and/or caspase proteolytic domains. Typically, such apoptotic polypeptides are capable of inducing an apoptotic signal upon dimerization. A skilled artisan will understand how to identify and incorporate these intracellular apoptotic domains (and other apoptosis-inducing domains) into an anti-idiotype polypeptide of the current disclosure. In some embodiments, the intracellular apoptotic domain of an anti-idiotype polypeptide can include one or more CARDs, DDs, DEDs, PYDs, and/or caspase proteolytic domains. In some embodiments, the intracellular apoptotic domain of an anti-idiotype polypeptide can include one or more means for performing the function of a CARD, DD, DED, PYD, and/or caspase proteolytic domain of a caspase, such as of a caspase 2, 8, 9, or 10. In some embodiments, the intracellular apoptotic domain can include one or more CARDs from Apaf-1, DARK, CED-4, CED-3, Dronc, CARMA1, Bcl-10, Nod1, Nod2, RIP2, ICEBERG, RIG-I, MDA5, MAV5, ASC, NALP1, caspase 1, caspase 2, caspase-5, and/or caspase 9, and/or functional fragments thereof. In some embodiments, the intracellular apoptotic domain can include one or more DDs from TNF-R1, Fas, p75, TRADD, FADD, RIP, MyD88, IRAKs, Pelle, Tube, PIDD, RAIDD, and/or MALT1, and/or functional fragments thereof. In some embodiments, the intracellular apoptotic domain can include one or more DEDs from FADD, caspase 8, caspase 10, c-FLIP, v-FLIPs, MC159, PEA-15, DEDD, and/or DEDD2, and/or functional fragments thereof. In some embodiments, the intracellular apoptotic domain can include one or more PYDs from ASC, ASC2, NALP1, NALP1, NALP3, NALP4, NALP5, NALP6, NALP7, NALP8, NALP9, NALP10, NALP11, and/or NALP12, and/or functional fragments thereof. In some embodiments, the intracellular apoptotic domain can include one or more domains from caspase 1, caspase 2, caspase 3, caspase 4, caspase 5, caspase 6, caspase 7, caspase 8, caspase 9, caspase 10, caspase 11, caspase 12, caspase 13, caspase 14, FAS (CD95 or Apo-1 antigen), TNF-R1, Death Receptor 3 (DR3), Death Receptor 4 (DR4 or TRAIL Receptor-I), Death Receptor 5 (DR5 or TRAIL Receptor-II), FADD, APAF1, CRADD/RAIDD, ASC, Bcl-2 family members, Bax, Bak, RIPK3, and/or RIPK1-RHIM, and/or functional fragments thereof. Not to be limited by theory, Bax and Bak are pro-apoptotic Bcl-2 family members that can cause mitochondrial depolarization (or mislocalization of anti-apoptotic family members, like Bcl-xL or Bcl-2). Not to be limited by theory, the RIPK3 and RIPK1-RHIM domain can trigger a related form of pro-inflammatory cell death, called necroptosis, due to MLKL-mediated membrane lysis. In illustrative embodiments, the intracellular apoptotic domain includes one or more of a caspase 2 polypeptide, a caspase 8 polypeptide, a caspase 9 polypeptide, and/or a caspase 10 polypeptide. Caspase 2, caspase 8, caspase 9, and caspase 10 are also referred to herein as initiator caspases. In some embodiments, the intracellular domain of an anti-idiotype polypeptide includes a fusion polypeptide of one or more domains of an initiator caspase and one or more domains of an effector caspase. In some embodiments, the initiator caspase can be caspase 2, caspase 8, caspase 9, and/or caspase 10. In some embodiments, the effector caspase can be caspase 3, caspase 6, and/or caspase 7. In some embodiments, intracellular apoptotic domain of an anti-idiotype polypeptide can include at least one domain from one or more of caspase 2, caspase 8, caspase 9, or caspase 10, or a functional fragment thereof. In some embodiments, the functional fragment is an intracellular domain from a caspase polypeptide that lacks one or more domains. In some embodiments, the one or more domains lacking in the functional fragment can be CARDs, DDs, DEDs, PYDs, and/or caspase proteolytic domains. In some embodiments, the functional fragment is a caspase 2 polypeptide lacking a CARD. In some embodiments, the functional fragment is a caspase 8 polypeptide lacking a DED.

In some embodiments, the intracellular apoptotic domain of an anti-idiotype polypeptide includes one or more domains from a caspase 2 polypeptide. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of SEQ ID NO:680. In some embodiments, the intracellular apoptotic domain includes all domains of the protein of SEQ ID NO:680. In some embodiments, the intracellular apoptotic domain includes one or more domains of the polypeptide of SEQ ID NO:680. In some embodiments, the intracellular apoptotic domain includes one or more domains of the polypeptide of SEQ ID NO:680. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of the CARD of caspase 2 (amino acids 32 to 118 of SEQ ID NO:680) and/or the CASc domain of caspase 2 (amino acids 192 to 447 of SEQ ID NO:680). In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:680, wherein the polypeptide sequence does not include a stretch of at least 10, 15, 20, or all the amino acids of the CARD (amino acids 32-118 of SEQ ID NO:680) and/or the CASc domain of caspase 2 (amino acids 192 to 447 of SEQ ID NO:680). In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of amino acids 327 to 452 of SEQ ID NO:680 or of amino acids 359 to 452 of SEQ ID NO:680.

In some embodiments, the intracellular apoptotic domain of an anti-idiotype polypeptide includes one or more domains from a caspase 8 polypeptide. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of SEQ ID NO:681. In some embodiments, the intracellular apoptotic domain includes all domains of the protein of SEQ ID NO:681. In some embodiments, the intracellular apoptotic domain includes one or more domains of the protein of SEQ ID NO:681. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of the DED of caspase 8 (amino acids 3 to 84 of SEQ ID NO:681), the DD of caspase 8 (amino acids 134 to 212 of SEQ ID NO:681), and/or CASc domain of caspase 8 amino acids 242 to 494 of SEQ ID NO:681. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:681, wherein the polypeptide sequence does not include a stretch of at least 10, 15, 20, or all the amino acids of the DED of caspase 8 (amino acids 3 to 84 of SEQ ID NO:681), the DD of caspase 8 (amino acids 134 to 212 of SEQ ID NO:681), and/or CASc domain of caspase 8 amino acids 242 to 494 of SEQ ID NO:681. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of amino acids 384 to 496 of SEQ ID NO:681 or of amino acids 411 to 496 SEQ ID NO:681.

In some embodiments, the intracellular apoptotic domain of an anti-idiotype polypeptide includes one or more domains from a caspase 9 polypeptide. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of the amino acids of SEQ ID NO:682. In some embodiments, the intracellular apoptotic domain includes all domains of the protein of SEQ ID NO:682. In some embodiments, the intracellular apoptotic domain includes one or more domains of the protein of SEQ ID NO:682. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of the CARD of caspase 9 (amino acids 17 to 90 of SEQ ID NO:682) and/or the CASc domain of caspase 9 (amino acids 152 to 414 of SEQ ID NO:682). In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:682, wherein the polypeptide sequence does not include a stretch of at least 10, 15, 20, or all the amino acids of the CARD of caspase 9 (amino acids 17 to 90 of SEQ ID NO:682) and/or the CASc domain of caspase 9 (amino acids 152 to 414 of SEQ ID NO:682). In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of amino acids 294 to 416 of SEQ ID NO:682 or of amino acids 336 to 416 of SEQ ID NO:682.

In some embodiments, the intracellular apoptotic domain of an anti-idiotype polypeptide includes one or more domains from a caspase 10 polypeptide. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of SEQ ID NO:683. In some embodiments, the intracellular apoptotic domain includes all domains of the protein of SEQ ID NO:683. In some embodiments, the intracellular apoptotic domain includes one or more domains of the protein of SEQ ID NO:683. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of the DD of caspase 10 (amino acids 18 to 99 of SEQ ID NO:683), the DED of caspase 10 (amino acids 112 to 190 of SEQ ID NO:683), and/or the CASc domain of caspase 10 (amino acids 233 to 474 of SEQ ID NO:683). In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:683, wherein the polypeptide sequence does not include a stretch of at least 10, 15, 20, or all the amino acids of the DD of caspase 10 (amino acids 18 to 99 of SEQ ID NO:683), the DED of caspase 10 (amino acids 112 to 190 of SEQ ID NO:683), and/or the CASc domain of caspase 10 (amino acids 233 to 474 of SEQ ID NO:683). In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of amino acids 365 to 478 of SEQ ID NO:683 or of amino acids 388 to 478 of SEQ ID NO:683.

In some embodiments, the intracellular apoptotic domain of an anti-idiotype polypeptide can include one or more means for activating an initiator caspase or an effector caspase, or both. In some embodiments, the intracellular apoptotic domain of an anti-idiotype polypeptide can include one or more means for binding to a death domain. In some embodiments, the intracellular apoptotic domain of an anti-idiotype polypeptide can include one or more means for performing the function of a death domain of caspase 2, 3, 9, or 10, or FAS or a TNF receptor ICD. In some illustrative embodiments, these anti-idiotype polypeptides further comprise a dimerizing moiety, wherein the dimerizing moiety is constitutively dimerized. In some embodiments, the intracellular apoptotic domain of an anti-idiotype polypeptide intracellular domain includes one or more domains from a FAS (CD95 or Apo-I antigen) polypeptide. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of SEQ ID NO:684. In some embodiments, the intracellular apoptotic domain includes all domains of the protein of SEQ ID NO:684. In some embodiments, the intracellular apoptotic domain includes one or more domains of the protein of SEQ ID NO:684. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of the DD of FAS polypeptide (amino acids 257 to 341 of SEQ ID NO:684). In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:684, wherein the polypeptide sequence does not include a stretch of at least 10, 15, 20, or all the amino acids of the DD of FAS polypeptide (amino acids 257 to 341 of SEQ ID NO:684).

In some embodiments, the intracellular apoptotic domain of an anti-idiotype polypeptide includes one or more domains from a TNF-R1 (Tumor necrosis factor receptor superfamily member 1) polypeptide. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of SEQ ID NO:685. In some embodiments, the intracellular apoptotic domain includes all domains of the protein of SEQ ID NO:685. In some embodiments, the intracellular apoptotic domain includes one or more domains of the protein of SEQ ID NO:685. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of the DD of TNF-R1 polypeptide (amino acids 358 to 438 of SEQ ID NO:685). In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:685, wherein the polypeptide sequence does not include a stretch of at least 10, 15, 20, or all the amino acids of the DD of TNF-R1 polypeptide (amino acids 358 to 438 of SEQ ID NO:685).

In some embodiments, the intracellular apoptotic domain of an anti-idiotype polypeptide includes one or more domains from a DR-3 (Tumor necrosis factor receptor superfamily member 25) polypeptide. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of SEQ ID NO:686. In some embodiments, the intracellular apoptotic domain includes all domains of the protein of SEQ ID NO:686. In some embodiments, the intracellular apoptotic domain includes one or more domains of the protein of SEQ ID NO:686. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of the DD of DR-3 polypeptide (amino acids 32 to 145 of SEQ ID NO:686). In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:686, wherein the polypeptide sequence does not include a stretch of at least 10, 15, 20, or all the amino acids of the DD of DR-3 polypeptide (amino acids 32 to 145 of SEQ ID NO:686).

In some embodiments, the intracellular apoptotic domain of an anti-idiotype polypeptide includes one or more domains from a DR-4 (Tumor necrosis factor receptor superfamily member 10A; TRAIL Receptor-I) polypeptide. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of SEQ ID NO:687. In some embodiments, the intracellular apoptotic domain includes all domains of the protein of SEQ ID NO:687. In some embodiments, the intracellular apoptotic domain includes one or more domains of the protein of SEQ ID NO:687. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of the DD of DR-4 polypeptide (amino acids 367 to 454 of SEQ ID NO:687). In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:687, wherein the polypeptide sequence does not include a stretch of at least 10, 15, 20, or all the amino acids of the DD of DR-4 polypeptide (amino acids 367 to 454 of SEQ ID NO:687).

In some embodiments, the intracellular apoptotic domain of an anti-idiotype polypeptide includes one or more domains from a DR-5 (Tumor necrosis factor receptor superfamily member 10B; TRAIL Receptor-II) polypeptide. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of SEQ ID NO:688. In some embodiments, the intracellular apoptotic domain includes all domains of the protein of SEQ ID NO:688. In some embodiments, the intracellular apoptotic domain includes one or more domains of the protein of SEQ ID NO:688. In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, or all of the DD of DR-5 polypeptide (amino acids 341 to 428 of SEQ ID NO:688). In some embodiments, the intracellular apoptotic domain includes a polypeptide sequence with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:688, wherein the polypeptide sequence does not include a stretch of at least 10, 15, 20, or all the amino acids of the DD of DR-5 polypeptide (amino acids 341 to 428 of SEQ ID NO:688).

In some embodiments, the anti-idiotype polypeptide includes a cleavable signal. In some embodiments, the cleavable signal can include a binding-induced proteolytic cleavage site of a Notch receptor. In some embodiments, the cleavable signal can include a gamma secretase recognition sequence.

In some embodiments, the anti-idiotype polypeptide includes a Notch receptor that includes an extracellular domain having a recognition site for a target antibody or a target antibody mimetic, a transmembrane domain having a binding induced proteolytic cleavage site, and one or more intracellular domains. In some embodiments, the intracellular domains can include one or more transcription factor. Not to be limited by theory, engagement of the extracellular domain of the Notch receptor with its ligand (that can be a target antibody or antibody mimetic) leads to intramembrane proteolysis through dimerization of the receptor (sequential proteolysis by a disintegrin and metalloproteinase (ADAM) metalloprotease and the gamma-secretase complex). The induced cleavage of the receptor releases the intracellular fragment of Notch. In some embodiments, the notch receptor, which is required for dimerization, can be absent from the anti-idiotype polypeptide. In further embodiments, the anti-idiotype polypeptide can include an extracellular recognition domain, which can provide dimerization upon addition of the antibody or antibody mimetic. The Notch intracellular domain is a transcriptional regulator that is released from the membrane upon dimerization and translocates into the nucleus to activate target genes that play key roles in cell-cell signaling during development. In some embodiments, the intracellular domain of an anti-idiotype polypeptide can include the Notch intracellular domain. Such intracellular domains on anti-idiotype polypeptides that regulate transcription are referred to herein as intracellular transcriptional domains, and can increase or decrease expression of target genes to which the intracellular transcriptional domains bind. In some embodiments, the intracellular domain is a transcriptional activator and increases expression. In some embodiments, the intracellular domain is a transcriptional repressor and decreases expression. In some embodiments, the intracellular domain of an anti-idiotype polypeptide can be an artificial transcription factor. In some embodiments, the artificial transcription factor can include domains from zinc-finger nucleases, Ga14, and/or tetR, or any other transcription factor known in the art. In some embodiments, the intracellular domain is a site-specific nuclease. The site-specific nuclease can be one or more of zinc finger nuclease (ZFN), Transcription activator-like effector nucleases (TALEN), CRISPR/Cas9 system. In some embodiments, the intracellular domain includes one or more of the domains of a Caspase 9 polypeptide. In some embodiments, the intracellular domain is a recombinase. In some embodiments, the intracellular domain is an inhibitory immunoreceptor. In some embodiments, the intracellular domain is an activating immunoreceptor. In some embodiments, release of the intracellular domain modulates proliferation of the cell. In some embodiments, release of the intracellular domain modulates apoptosis in the cell. In some embodiments, release of the intracellular domain induces cell death by a mechanism other than apoptosis. In some embodiments, release of the intracellular domain modulates gene expression in the cell through transcriptional regulation, chromatin regulation, translation, trafficking or post-translational processing. In some embodiments, release of the intracellular domain modulates differentiation of the cell. In some embodiments, release of the intracellular domain modulates migration of the cell. In some embodiments, release of the intracellular domain modulates the expression and secretion of a molecule from the cell. In some embodiments, release of the intracellular domain modulates adhesion of the cell to a second cell or to an extracellular matrix. In some embodiments, release of the intracellular domain induces de novo expression a gene product in the cell. In some embodiments, release of the intracellular domain induces de novo expression a gene product in the cell, wherein the gene product is a transcriptional activator, a transcriptional repressor, a chimeric antigen receptor, a second chimeric Notch receptor polypeptide, a translation regulator, a cytokine, a hormone, a chemokine, or an antibody. Some of the examples of such notch receptors and the intracellular domain which gets released upon the binding of the target antibody or antibody mimetic to the notch receptor provided in the U.S. Pat. US10590182B2 are to be incorporated herein.

In some embodiments, the anti-idiotype polypeptide can include a cleavable signal that can be one or more gamma secretase substrate sequences that are recognizable by gamma secretase for cleavage. The gamma secretase substrate sequences (also known as the recognition sequences for gamma secretase) are the sequences from the substrates of the gamma-secretase owing to which the substrate gets cleaved. In some embodiments, the one or more gamma secretase substrate sequences can be from one or more polypeptides such as ERBB4, INSR, IGF1R, CSF1R, VEGFR1, VEGFR2, VEGFR3, FGFR3, FGFR4, PTK7, TRKA, TRKB, MUSK, MET, AXL, MER, TYRO3, TIE1, EPHA2, EPHA4, EPHA5, EPHA7, EPHB2, EPHB3, EPHB4, EPHB6, RYK, Alcadein α, Alcadein β, Alcadein γ, APLP1, APLP2, APP, CD44, E-Cadherin, EpCAM, GHR, IL-1R2, Neuregulin-1, Notch-1, Notch-2, Notch-3, Notch-4, P75 NTR, Podoplanin, PTK7, and Syndecan-3.

In some embodiments, the anti-idiotype polypeptide can include gamma secretase substrate sequence of Alcadein a polypeptide. In some embodiments, the anti-idiotype polypeptide includes a polypeptide sequence with at least 50%, 60%, 70%, 75%, 80%, 85%, 90%, 95%, 96%, 97%, 98%, 99% or 100% sequence identity to a stretch of at least 10, 15, 20, or all the amino acids of SEQ ID NO:689. In some embodiments, the anti-idiotype polypeptide can include gamma secretase substrate sequence of Alcadein β polypeptide. In some embodiments, the anti-idiotype polypeptide includes a polypeptide sequence with at least 50%, 60%, 70%, 75%, 80%, 85%, 90%, 95%, 96%, 97%, 98%, 99% or 100% sequence identity to a stretch of at least 10, 15, 20, or all the amino acids of SEQ ID NO:690. In some embodiments, the anti-idiotype polypeptide can include gamma secretase substrate sequence of (3 Alcadein γ polypeptide. In some embodiments, the anti-idiotype polypeptide includes a polypeptide sequence with at least 50%, 60%, 70%, 75%, 80%, 85%, 90%, 95%, 96%, 97%, 98%, 99% or 100% sequence identity to a stretch of at least 10, 15, 20, or all the amino acids of SEQ ID NO:691. In some embodiments, the anti-idiotype polypeptide can include gamma secretase substrate sequence of APLP1 polypeptide. In some embodiments, the anti-idiotype polypeptide includes a polypeptide sequence with at least 50%, 60%, 70%, 75%, 80%, 85%, 90%, 95%, 96%, 97%, 98%, 99% or 100% sequence identity to a stretch of at least 10, 15, 20, or all the amino acids of SEQ ID NO:692. In some embodiments, the anti-idiotype polypeptide can include gamma secretase substrate sequence of APLP2 polypeptide. In some embodiments, the anti-idiotype polypeptide includes a polypeptide sequence with at least 50%, 60%, 70%, 75%, 80%, 85%, 90%, 95%, 96%, 97%, 98%, 99% or 100% sequence identity to a stretch of at least 10, 15, 20, or all the amino acids of SEQ ID NO:693. In some embodiments, the anti-idiotype polypeptide can include gamma secretase substrate sequence of Notch-1 polypeptide. In some embodiments, the anti-idiotype polypeptide includes a polypeptide sequence with at least 50%, 60%, 70%, 75%, 80%, 85%, 90%, 95%, 96%, 97%, 98%, 99% or 100% sequence identity to a stretch of at least 10, 15, 20, or all the amino acids of SEQ ID NO:694. In some embodiments, the anti-idiotype polypeptide can include gamma secretase substrate sequence of Notch-2 polypeptide. In some embodiments, the anti-idiotype polypeptide includes a polypeptide sequence with at least 50%, 60%, 70%, 75%, 80%, 85%, 90%, 95%, 96%, 97%, 98%, 99% or 100% sequence identity to a stretch of at least 10, 15, 20, or all the amino acids of SEQ ID NO:695. In some embodiments, the anti-idiotype polypeptide can include gamma secretase substrate sequence of Notch-3 polypeptide. In some embodiments, the anti-idiotype polypeptide includes a polypeptide sequence with at least 50%, 60%, 70%, 75%, 80%, 85%, 90%, 95%, 96%, 97%, 98%, 99% or 100% sequence identity to a stretch of at least 10, 15, 20, or all the amino acids of SEQ ID NO:696. In some embodiments, the anti-idiotype polypeptide can include gamma secretase substrate sequence of Notch-4 polypeptide. In some embodiments, the anti-idiotype polypeptide includes a polypeptide sequence with at least 50%, 60%, 70%, 75%, 80%, 85%, 90%, 95%, 96%, 97%, 98%, 99% or 100% sequence identity to a stretch of at least 10, 15, 20, or all the amino acids of SEQ ID NO:697. Linkers

In some embodiments, the anti-idiotype polypeptide can include a linker between any two adjacent domains. For example, a linker can be between the transmembrane domain and the first intracellular domain. As another example, the extracellular recognition domain of the anti-idiotype can be an antibody and a linker can be between the heavy chain and the light chain. As another example, a linker can be between the extracellular recognition domain and the transmembrane domain and another linker can be between the transmembrane domain and the intracellular domain. As another example, a linker can be between a first intracellular domain and a second intracellular domain. As another example, the linker can be between the extracellular recognition domain and the intracellular domain.

The linker peptide may have any of a variety of amino acid sequences. Proteins can be joined by a spacer peptide, generally of a flexible nature, although other chemical linkages are not excluded. A linker can be a peptide of between about 1 and about 100 amino acids in length, or between about 1 and about 25 amino acids in length. These linkers can be produced by using synthetic, linker-encoding oligonucleotides to couple the proteins. Peptide linkers with a degree of flexibility can be used. The linking peptides may have virtually any amino acid sequence, bearing in mind that suitable linkers will have a sequence that results in a generally flexible peptide. The use of small amino acids, such as glycine and alanine, are typically of use in creating a flexible peptide. The creation of such sequences is routine to those of skill in the art.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, (GSGGS)ₙ, (GGS)ₙ, (GGGS)ₙ, and (GGGGS)ₙ where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited GGGGSGGGGS (SEQ ID NO:674), GGGGSGGGGSGGGGS (SEQ ID NO:63), GGGGSGGGGSGGGGSGGGGS (SEQ ID NO:372), GGGGSGGGGSGGGGSGGGGSGGGGS (SEQ ID NO:675), GGGGSGGGGSGGGGSGGGGSGGGGSGGGGS (SEQ ID NO:64), GGSSRSS (SEQ ID NO:673), GGGGSGGGSGGGGS (SEQ ID NO:65), GGSG (SEQ ID NO:66), GGSGG (SEQ ID NO:67), GSGSG (SEQ ID NO:68), GSGGG (SEQ ID NO:69), GGGSG (SEQ ID NO:70), GSSSG (SEQ ID NO:71), and the like. The ordinarily skilled artisan will recognize that design of a peptide conjugated to any elements described above can include linkers that are all or partially flexible, such that the linker can include a flexible linker as well as one or more portions that confer less flexible structure.

### Anti-Idiotype Fusion Polypeptides

Anti-idiotype polypeptides can function as single polypeptides, or can function as fusions to other polypeptides. The fusion polypeptides can include only the extracellular recognition domains of one or more anti-idiotype polypeptides or the fusion polypeptides can further include one or more of the other domains of the anti-idiotype polypeptides. Based on the presence of the extracellular recognition domain from the anti-idiotype polypeptide, the fusion polypeptides can have different activities based on the presence or absence of the target antibody or antibody mimetic. For example, a constitutively active lymphoproliferative element can include an extracellular recognition domain from an anti-idiotype polypeptide that recognizes a target antibody. In the absence of the target antibody, the constitutively active lymphoproliferative element can promote proliferation and/or survival of the cell expressing the fusion polypeptide. In the presence of the target antibody, for example a target antibody that induces ADCC, binding of the target antibody to the extracellular recognition domain can result in death of the cell expressing the fusion polypeptide with the constitutive lymphoproliferative element. In some embodiments, the extracellular recognition domain of a fusion polypeptide can perform any of the functions of an anti-idiotype polypeptide disclosed herein, including, for example, acting as a safety switch and acting as an activity modulator. In such fusion polypeptides, the membrane association domain of the anti-idiotype polypeptide can be provided by the polypeptide to which the anti-idiotype polypeptide is fused.

In some embodiments an anti-idiotype polypeptide, or the anti-idiotype extracellular recognition domain of an anti-idiotype polypeptide, is expressed as part of a fusion polypeptide. In some embodiments, the fusion polypeptide can be a fusion between the anti-idiotype polypeptide or the extracellular recognition domain of an anti-idiotype polypeptide and a first engineered signaling polypeptide, which are disclosed in more detail elsewhere herein. Thus, in some embodiments, the the anti-idiotype polypeptide or the extracellular recognition domain of an anti-idiotype polypeptide is fused to a lymphoproliferative element, CAR, and/or a recombinant TCR. In some embodiments, the the anti-idiotype polypeptide or the extracellular recognition domain of an anti-idiotype polypeptide is fused to a cytokine. In some embodiments, the fusion polypeptide can further include one or more of the other domains of the anti-idiotype polypeptides.

In some embodiments, the fusion polypeptide including an anti-idiotype polypeptide can further include a CAR. In some embodiments, the fusion polypeptide can include a recombinant TCR. In embodiments wherein the anti-idiotype polypeptide is fused to a CAR or a recombinant TCR, the extracellular recognition domain of the anti-idiotype polypeptide can be expressed as part of the antigen-specific binding region (ASTR, as discussed elsewhere herein) of the CAR (e.g., a bispecific antibody) or the antigen binding site of the recombinant TCR, or the extracellular recognition domain can be expressed as part of a separate domain on the fusion polypeptide. For example, the CAR and the extracellular recognition domain from the anti-idiotype polypeptide can each be one part of a bispecific antibody that is attached to the other domains of the fusion polypeptide.

In some embodiments, an anti-idiotype polypeptide, for example a safety switch or an activity modulator, is expressed fused to a lymphoproliferative element, to form a fusion polypeptide. Thus, in some embodiments, the fusion polypeptide can include a lymphoproliferative element. Such constructs provide the advantage, especially in combination with other “space saving” elements provided herein, of taking up less genomic space on an RNA genome compared to separate polypeptides. The lymphoproliferative element of a fusion polypeptide can be any of the lymphoproliferative elements disclosed elsewhere herein. In some embodiments, the lymphoproliferative element of the fusion polypeptide can include one or more or all of the domains, motifs, and/or mutations of the intracellular signaling domains disclosed herein or otherwise known to induce proliferation and/or survival of T cells and/or NK cells. In some embodiments, the lymphoproliferative element of a fusion polypeptide is constitutively active. In some embodiments, the lymphoproliferative element of a fusion polypeptide is an inducible lymphoproliferative element. In some such embodiments, binding of the target antibody or antibody mimetic to the extracellular recognition domain on the fusion polypeptide dimerizes two intracellular signaling domains of the inducible lymphoproliferative element to drive proliferation of cells such as T cells and/or NK cells. In some embodiments, the inducible lymphoproliferative element of a fusion polypeptide activates proliferative and/or survival signaling after binding of the anti-idiotype extracellular recognition domain to the target antibody or antibody mimetic, as also discussed in the “Activity modulators” section herein. In some embodiments, a fusion polypeptide that includes an inducible lymphoproliferative element can include a dimerization domain besides the anti-idiotype extracellular recognition domain. In illustrative embodiments, a fusion polypeptide that includes an inducible lymphoproliferative element does not include a dimerization domain besides the anti-idiotype extracellular recognition domain.

In one illustrative embodiment, an eTag is expressed as a fusion polypeptide, fused the 5′ terminus of the c-Jun domain (SEQ ID NO: 104), a transmembrane domain from CSF2RA (SEQ ID NO: 129), a first intracellular domain from MPL (SEQ ID NO:283), and a second intracellular domain from CD40 (SEQ ID NO:208). When expressed as a polypeptide not fused to a CAR or lymphoproliferative element, the cell tag may be associated with the cell membrane via its natural membrane attachment sequence or via a heterologous membrane attachment sequence such as a GPI-anchor or transmembrane sequence. In illustrative embodiments cell tags are expressed on the T cell and/or NK cell but are not expressed on the replication incompetent recombinant retroviral particles. In some embodiments, polynucleotides, polypeptides, and cells comprise 2 or more safety switches.

### Cell Formulations and Methods of Administration

In some embodiments such as embodiments in which the samples do not undergo a PBMC isolation or granulocyte depletion procedure, at least 5%, at least 10%, at least 20%, at least 25%, at least 30%, at least 40%, at least 50%, or at least 75% of the neutrophils, basophils, and/or eosinophils present in a blood sample that is subjected to a method for modifying herein, are present in the cell formulation, including at the time of the optional delivery (i.e., administering) step. In some embodiments such as those embodiments in which the samples do not undergo a B cell depletion procedure, at least 5%, at least 10%, at least 20%, at least 25%, at least 30%, at least 40%, at least 50%, or at least 75% of the B cells present in a blood sample that is subjected to a method for modifying herein, are present in the cell formulation, including at the time of the optional delivery step. In some embodiments such as those embodiments in which the samples do not undergo a monocyte depletion procedure, at least 5%, at least 10%, at least 20%, at least 25%, at least 30%, at least 40%, at least 50%, or at least 75% of the monocytes present in a blood sample that is subjected to a method for modifying herein, are present in the cell formulation, including at the time of the optional delivery step.

In some embodiments, and in illustrative embodiments in which the cell formulation is administered subcutaneously or intramuscularly, the volume of the cell formulation including the modified lymphocytes is less than traditional CAR-T methods, which typically are infusion-delivery methods, and can be less than, or less than about 1 ml, about 2 ml, about 3 ml, about 4 ml, about 5 ml, about 10 ml, about 15 ml, about 20 ml, or about 25 ml.

The short contacting time in certain embodiments results in many of the modified lymphocytes in cell formulations herein, having on their surfaces, binding polypeptides, fusogenic polypeptides, and in some embodiments T cell activation elements that originated on the surface of retroviral particles, either through association with the recombinant retroviral particles or by fusion of the retroviral envelopes with the plasma membranes, including at the time of the optional delivery step. In some embodiments, at least 10%, 15%, 20%, 25%, 30%, 35%, 40%, 45%, 50%, 55%, 60%, 65%, 70%, 75%, 80%, 85%, or 90% of the modified lymphocytes in the cell formulation include a pseudotyping element and/or a T cell activation element, e.g., a T cell activating antibody. In some embodiments, the pseudotyping element and/or T cell activation element can be bound to the surface of the modified lymphocytes through, for example, a T cell receptor, CD28, OX40, 4-1BB, ICOS, CD9, CD53, CD63, CD81, CD82, and/or the pseudotyping element and/or T cell activation element can be present in the plasma membrane of the modified lymphocytes.

Cell formulations are provided herein, that include for example T cells and/or NK cells. Such formulations, in illustrative embodiments are provided by methods provided herein. Any of the cell formulations provided herein can include self-driving CAR-T cells. In one aspect, provided herein is a cell formulation comprising a population of self-driving CAR-T cells, such as modified, genetically modified, transcribed, transfected, and/or stably integrated self-driving CAR-T cells in a delivery solution.

Due to the advantageously short time lymphocytes are contacted with recombinant nucleic acid vectors and modified lymphocytes are ex vivo after such contacting in some illustrative embodiments provided herein, in these embodiments some or all of the T and NK cells do not yet express the recombinant nucleic acid or have not yet integrated the recombinant nucleic acid into the genome of the cell, and some of the retroviral particles in embodiments including these, may be associated with, but may have not fused with the target cell membrane, before being used or included in any of the methods or compositions provided herein, including, but not limited to, being introduced or reintroduced back into a subject, or before being used to prepare a cell formulation. Thus, various cell formulation aspects and embodiments are provided herein that can be produced, for example, from these illustrative methods provided herein, such as for example, rapid point of care methods that in illustrative embodiments involve subcutaneous administration. Such cell formulations, including but not limited to those set out immediately below and in the Exemplary Embodiments section herein, can exist at the time of collection of cells after they are contacted with a recombinant retroviral vector and optionally rinsed, and can exist up to and including at the time of administration to a subject, in illustrative embodiments subcutaneously.

In some embodiments, provided herein are cell formulations comprising T cells and/or NK cells, wherein less than 90%, 80%, 75%, 70%, 60%, 50%, 40%, 30%, 25%, 20%, 10%, or 5% of the cells in the cell formulation are T cells and/or NK cells. In some embodiments, cell formulations comprising lymphocytes, NK cells, and/or T cells, are provided wherein at least 4%, 5%, 10%, 15%, 20%, 25%, 30%, 35%, 40%, 45%, 50%, 55%, 60%, 65%, 70%, 75%, 80%, 85%, 90%, or 95% of the lymphocytes, NK cells, and/or in illustrative embodiments T cells in the cell formulation are modified cells, for example, modified with polynucleotides comprising nucleic acids that encode anti-idiotype polypeptides provided herein. Such polynucleotides can optionally encode a CAR, TCR, inhibitory RNA, or LE, as provided herein. In some embodiments, between 4%, 5%, 10%, 15%, 20%, 25%, 30%, 35%, 40%, 45%, 50%, 55%, 60%, 65%, and 70% of the lymphocytes are modified on the low end of the range and 10%, 15%, 20%, 25%, 30%, 35%, 40%, 45%, 50%, 55%, 60%, 65%, 70%, 75%, 80%, 85%, 90%, and 95% of the lymphocytes are modified cells on the high end of the range, for example between 5% and 95%, 10% and 90%, 25% and 75%, and 25% and 95%. In some embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the modified lymphocytes within the cell formulation are not genetically modified, transduced, or stably transfected. In some embodiments, between 5%, 10%, 15%, 20%, 25%, 30%, 35%, 40%, 45%, 50%, 55%, 60%, 65%, and 70% of the modified lymphocytes are not genetically modified, transduced, or stably transfected on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified lymphocytes are not genetically modified, transduced, or stably transfected on the high end of the range, for example between 5% and 95%, 10% and 90%, 25% and 75%, and 25% and 95%. In some embodiments, the polynucleotide of genetically modified lymphocytes can be either extrachromosomal or integrated into the genome in these cell formulations that are formed after contacting and incubation, and at the time of optional administration. In some embodiments of these cell formulations, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the genetically modified lymphocytes have an extrachromosomal polynucleotide. In some embodiments, between 5%, 10%, 15%, 20%, 25%, 30%, 35%, 40%, 45%, 50%, 55%, 60%, 65%, and 70% of the modified or genetically modified lymphocytes have an extrachromosomal polynucleotide on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified or genetically modified lymphocytes have an extrachromosomal polynucleotide on the high end of the range, for example between 5% and 95%, 10% and 90%, 25% and 75%, and 25% and 95%. In some embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the modified or genetically modified lymphocytes are not transduced or stably transfected in these cell formulations, for example as a result of methods for genetically modifying T cells and/or NK cells provided herein. In some embodiments, between 5%, 10%, 15%, 20%, 25%, 30%, 35%, 40%, 45%, 50%, 55%, 60%, 65%, and 70% of the modified or genetically modified lymphocytes are not transduced on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified or genetically modified lymphocytes are not transduced or stably transfected on the high end of the range, for example between 5% and 95%, 10% and 90%, 25% and 75%, and 25% and 95%.

. Not to be limited by theory, unlike traditional CAR-T cell processing methods where cells are cultured ex-vivo for days or weeks and many cell divisions, in illustrative methods provided herein where T cells and/or NK cells are contacted with retroviral particles to modify the T cells and/or N cells within hours of delivery, some or most of the reverse transcriptase and integrase present within the retroviral particles that moves into a T cell and/or NK cell after it fuses with a retroviral particle, would still be present in the modified T cells and/or NK cells at the time of delivery.

The volume of cell formulation or other solution administered varies depending on the route of administration, as provided elsewhere herein. Cell formulations injected subcutaneously or intramuscularly typically have smaller volumes than those delivered via infusion. In some embodiments, the volume of the cell formulation or other solution including a suspension of the modified, and in illustrative embodiments genetically modified lymphocytes is not more than 1 ml, 2 ml, 3 ml, 4 ml, 5 ml, 10 ml, 15 ml, 20 ml, 25 ml, 30 ml, 35 ml, 40 ml, 45 ml, or 50 ml. In some embodiments, the volume of the cell formulation or other solution including a suspension of the modified lymphocytes can be between 0.20 ml, 0.25 ml, 0.5 ml, 1 ml, 2 ml, 3 ml, 4 ml, 5 ml, 10 ml, 15 ml, 20 ml, or 25 ml on the low end of the range and 0.5 ml, 1 ml, 2 ml, 3 ml, 4 ml, 5 ml, 10 ml, 15 ml, 20 ml, 25 ml, 30 ml, 35 ml, 40 ml, 45 ml, or 50 ml., 30 ml, 35 ml, 40 ml, 45 ml, 50 ml, 75 ml, 100 ml, 125 ml, 250 ml, 500 ml, or 1000 ml on the high end of the range. Thus, as non-limiting examples, the volume can be between 0.2 ml and 10 ml, 0.5 ml and 10 ml, 0.5 and 2 ml, 1 ml and 250 ml, 1 ml and 100 ml, 10 ml and 100 ml, or 1 ml and 10 ml. In certain illustrative embodiments, less than 10 ml, between 1 ml and 25 ml, and in illustrative embodiments between 1 ml and 3 ml, between 1 ml and 5 ml, or between 1 ml and 10 ml of a cell formulation that includes modified lymphocytes in delivery solution are administered subcutaneously or intramuscularly. In illustrative embodiments, the volume of the solution including the modified lymphocytes can be between 0.20 ml, 0.25 ml, 0.5 ml, 1 ml, 2 ml, 3 ml, 4 ml, and 5 ml on the low end of the range and 0.5 ml, 1 ml, 2 ml, 3 ml, 4 ml, 5 ml, 10 ml, 15 ml, 20 ml, 25 ml, 30 ml, 35 ml, 40 ml, 45 ml, and 50 ml on the high end of the range. In an exemplary embodiment, a 70 kg subject is dosed at 1.0 × 10⁶ T cells/kg by administering 1 ml of a delivery formulation of T cells at 7.0 × 10⁷ cells/ml subcutaneously. In some embodiments, the solution can include hyaluronidase when the volume of the solution is at least 2 ml, 3 ml, 4 ml, 5 ml, 10 ml, 15 ml, 20 ml, or 25 ml. In embodiments herein wherein lymphocytes are filtered especially after they are modified, and/or especially where transduction is performed on top of a filter, the delivery solution can be used to resuspend and/or elute cells from the filter in volumes that can be those provided above. As such, in some embodiments, a delivery solution provided herein is an elution solution.

In some embodiments, modified and in illustrative embodiments genetically modified lymphocytes are introduced or reintroduced into the subject by intradermal, intratumoral or intramuscular administration and in illustrative embodiments, subcutaneous administration using a cell formulation present in a subcutaneous delivery device, such as a sterile syringe that is adapted to deliver a solution subcutaneously. In some embodiments, a subcutaneous delivery device is used that holds a solution (e.g., a cell formulation herein) and has an open or openable end, which in illustrative embodiments is the open end of a needle, for administrating the solution (e.g., cell formulation) subcutaneously from the liquid holding portion of the device. Such subcutaneous delivery device is effective for, and in illustrative embodiments adapted for subcutaneous delivery, or effective to inject subcutaneously or adapted to inject subcutaneously. Non-limiting examples of subcutaneous delivery devices that are adapted to deliver a solution subcutaneously include subcutaneous catheters, such as indwelling subcutaneous catheters, such as for example, the Insuflon® (Becton Dickinson) and needless closed indwelling subcutaneous catheter systems, for example with wings, such as for example, the Saf-T-Intima® (Becton Dickinson). In some embodiments, the delivery device can include a pump, for example an infusion pump or a peristaltic pump. In some embodiments, the cell formulation is fluidly connected to any of the needles disclosed herein, for example a needle compatible with, effective for, adapted for, or adapted to deliver subcutaneously or effective to deliver subcutaneously

In some embodiments the delivery solution, a composition in the kit, or the cell formulation includes one or more cytokines such as IL-2, IL-7, IL-15, or IL-21, IL-21 and/or cytokine receptor agonists, such as an IL-15 agonist. In some embodiments the cytokine does not bind to a cytokine receptor included in the delivery solution, kit, or cell formulation; and/or does not bind to a cytokine receptor that is encoded by a polynucleotide in the delivery solution, cell formulation, or kit. In some embodiments, the cytokines can be modified cytokines that, not to be limited by theory, selectively activate complexes that drive proliferation. In illustrative embodiments, the modified cytokine is a modified IL-2, for example, a fusion protein with a circularly-permuted IL-2 with the extracellular domain of IL-2Rα (see, e.g., Lopes et al, J Immunother Cancer 2020 Apr; 8(1): e000673). In some embodiments, the cytokines, modified cytokines, or cytokine receptor agonists can also be administered in one or administrations separate from the cell formulation, before, contemporaneous to, or after the administration including the delivery solution or cell formulation. In some embodiments, two or more separate administrations can be in escalating doses. In some embodiments, two or more administrations can be at the same dose. In some embodiments, two or more administrations can include the same or different cytokines, modified cytokines, and or cytokine receptor agonists. In some embodiments, the separate administrations can be a series of 2, 3, 4, 5, 6, 7, 8, 9, 10, 11, 12, 13, 14, 15, 16, 17, 18, 19, 20, or 21 administrations. In some embodiments, the separate administrations occur on consecutive days.

In illustrative embodiments, cells in a first cell mixture, for example cells obtained from a subject, are modified with a recombinant nucleic acid vector encoding a target antigen, which can be referred to herein as “artificial antigen presenting cells” or “aAPCs”, and cells in a separate second cell mixture from the same subject are modified to express the CAR that binds the antigen. In some embodiments, where the modified cell that was modified with a vector encoding a target antigen is a T cell, the cell can be called a “T-APC” herein. Such modified T-APCs can include, as non-limiting examples, B cells, dendritic cells, and macrophages, and in illustrative embodiments dendritic cells and macrophages such as where a corresponding CAR-T target is a B cell cancer target, and can be generated using methods provided herein where reaction mixtures for modification (e.g., transduction) include a T cell binding polypeptide, such as a polypeptide directed to CD3. In further illustrative embodiments, the cell mixture is whole blood, isolated TNCs, isolated PBMCs. For example, the first cell mixture can be modified with a recombinant nucleic acid vector encoding a fusion protein of the extracellular domain of Her2 and the transmembrane domain of PDGF and the second cell mixture can be modified with a recombinant nucleic acid vector encoding a CAR directed to HER2. The cells can then be formulated into the delivery solution or otherwise administered to the subject at varying CAR effector cell-to-target-cell ratios. In some embodiments, the effector-to-target ratio at the time of formulation or administration is, or is about 10:1, about 9:1, about 8:1, about 7:1, about 6:1, about 5:1, about 4:1, about 3:1, about 2;1, about 1:1, about 1:2, about 1:3, about 1:5, about 1:6, about 1:7, about 1:8, about 1:9, or about 1:10. In illustrative embodiments, target cells are co-administered with the modified T and/or NK cells subcutaneously or intramuscularly.

### Synthetic RNA

Provided herein in another aspect, the delivery solution or the cell formulation includes synthetic RNA. In some embodiments the synthetic RNA includes inhibitory RNAs such as siRNAs directed to one or more targets. The targets for these inhibitory RNAs can be any of the targets for siRNAs or miRNAs disclosed elsewhere herein. In some embodiments, the synthetic RNA includes mRNA encoding for one or more proteins or peptides. In some embodiments, the mRNA encodes for one or more CARs. The CARs may be any CAR composition disclosed herein, including bispecific CARs that include an anti-id ERD as disclosed herein. In some embodiments, the mRNA can encode any anti-idiotype polypeptide disclosed herein. In some embodiments, the mRNA encodes for the target antibody to an anti-idiotype polypeptide disclosed herein. Such embodiment can have an advantage of providing a target antibody that can be cytotoxic when delivered in soluble form, but less or not cytotoxic when taken up by cells after in vivo administration of an mRNA encoding the target antibody. Thus, cells that take up and express the target antibody can become artificial antigen presenting cells for embodiments where the anti-idiotype polypeptide has an ICD that is the ICD of an LE, or of a CAR (e.g. a bi-speficic CAR).

In some embodiments, the mRNA encodes for one or more cytokines. In some embodiments, mRNA encodes for IL-2 or a functional variant thereof. In some embodiments, the mRNA encodes for IL-7 or a functional variant thereof. In some embodiments, the mRNA encodes for IL-15 or a functional variant thereof. In some embodiments, the mRNA encodes for IL-21 or a functional variant thereof. In some embodiments, the mRNA encodes one or more proteins or polypeptides that bind and activate a CAR. In some embodiments, the mRNA encodes for an antigen recognized by the ASTR of the CAR. In some embodiments, the mRNA encodes for HER2 or an extracellular domain of HER2. In some embodiments, the mRNA encodes for EGFR or an extracellular domain of EGFR. In some embodiments, the mRNA encodes for Axl or an extracellular domain of Axl. In some embodiments, the mRNA encodes for CD19 or an extracellular domain of CD19. In some embodiments, the mRNA encodes for CD22 or an extracellular domain of CD22. In some embodiments, the mRNA encodes for an antibody recognized by the ASTR of the CAR. In some embodiments, the MRNA encoding for an antibody recognized by the ASTR of the CAR is an anti-idiotype antibody directed to the antibody or scFv of the ASTR. In some embodiments, the mRNA encodes for an antibody that binds an epitope tag of the CAR and can cross-link two CARs as described elsewhere herein. In some embodiments, the mRNA encodes for one or more T and/or NK cell co-stimulatory proteins. Such co-stimulatory proteins may comprise one or more ligands or antibodies to a co-stimulatory receptor on T and/or NK cells. In some embodiments the co-stimulatory receptor is CD28. In some embodiments the co-stimulatory receptor is 4-1BB. In some embodiments, the mRNA encodes a protein or polypeptide that is soluble. In some embodiments, the mRNA encodes a protein or polypeptide that is membrane-bound. In some embodiments, the membrane-bound protein or polypeptide is operatively linked to a transmembrane domain. In some embodiments, the synthetic RNA includes both inhibitory RNAs such as siRNAs directed to one or more targets and mRNA encoding for one or more proteins or peptides.

A method for generating mRNA for use in the delivery solution or cell formulation may involve in vitro transcription of a template with specially designed primers, followed by PolyA addition, to produce a construct containing 3′ and 5′ untranslated sequence, a 5′ cap and/or IRES, the nucleic acid to be expressed, and a polyA tail, typically 50-200 bases in length. In some embodiments, the synthetic RNA is a naturally occurring, endogenous RNA for the nucleic acid of interest. In some embodiments, the RNA is not the naturally occurring, endogenous RNA for the nucleic acid of interest. In some embodiments, the RNA is modified to change the stability and/or translation efficiency of the RNA. In some embodiments, the 5′ UTR, 3′UTR, Kozak sequence, polyA tail is modified. In some embodiments, the RNA includes a 5′ cap. In some embodiments, the RNA is encapsulated in lipid-based carrier vehicles. One approach for assembling lipid nanocarriers includes directly mixing of a solution of lipids in ethanol with an aqueous solution of the nucleic acid to obtain lipid nanoparticles (LNPs). In some embodiments, the LNPs comprise PEG-conjugated lipid. PEG conjugated lipids prevent the aggregation during particle formation and allow the controlled manufacturing of particles with defined diameters in the range between approximately 50 nm and 150 nm. PEGylation of nanoparticles can have substantial disadvantages concerning safety and activity. The drawbacks associated with the use of PEGylated nanoparticles has stimulated the development of PEG alternatives. In some embodiments the LNPs do not comprise PEG. In some embodiments, the LNPs comprise poly(glycerol) (PGs), poly(oxazolines), sugar-based systems, and poly(peptides). In some embodiments, the polypeptides include polysarcosine (pSAR). In some embodiments, the LNPs comprise a dendritic cell targeting moiety. In some embodiments, the dendritic cell targeting moiety comprises mannose.

In some embodiments, the RNA can be added to a cell formulation comprising, or co-administered with, modified and/or genetically modified T cells and/or NK cells in cell formulations and methods provided herein. In some embodiments, the RNA is added to the isolated blood of a subject and processed in parallel with the T cells and/or NK cells. In some embodiments, the RNA can be formulated separately from the modified and/or genetically modified T cells and/or NK cells. The synthetic RNA may be delivered by any means known in the art for therapeutic delivery of RNA. In some embodiments, the RNA is delivered intravenously. In some embodiments, the RNA is delivered intraperitoneally. In some embodiments, the RNA is delivered intramuscularly. In some embodiments, the RNA is delivered intratumorally. In some embodiments, the RNA is delivered intradermally. In illustrative embodiments, the RNA is delivered subcutaneously. In some embodiments, the RNA is delivered at the same site as the site of administration of the modified and/or genetically modified T cells and/or NK cells. In some embodiments, the RNA is delivered at a site adjacent to the site of administration of the modified and/or genetically modified T cells and/or NK cells. In some embodiments, the RNA is administered once. In some embodiments, the RNA is administered, 2, 3, 4, 5, 6 or more times.

### Recombinant Retroviral Particles

Recombinant retroviral particles are disclosed in methods and compositions provided herein, for example, to modify cells, as non-limiting examples human cells, primary cells, T cells and/or NK cells to make genetically modified and/or transduced cells, human cells, primary cells, T cells and/or NK cells. The recombinant retroviral particles are themselves aspects of the present invention. Typically, the recombinant retroviral particles included in aspects provided herein, are replication incompetent, meaning that a recombinant retroviral particle cannot replicate once it leaves the packaging cell. In fact, unless indicated otherwise herein, retroviral particles are replication incompetent, and if such retroviral particles include nucleic acids in their genome that are not native to the retrovirus, they are “recombinant retroviral particles.” In illustrative embodiments, the recombinant retroviral particles are lentiviral particles.

Provided herein in some aspects are replication incompetent recombinant retroviral particles for use in transducing cells, typically lymphocytes and illustrative embodiments T cells and/or NK cells. The replication incompetent recombinant retroviral particles can include an envelope protein. In some embodiments, the envelope protein can be a pseudotyping element. In some embodiments, the envelope protein can be an activation element. In some embodiments, the replication incompetent recombinant retroviral particles include both a pseudotyping element and an activation element. The replication incompetent recombinant retroviral particles can include any of the pseudotyping elements discussed elsewhere herein. In some embodiments, the replication incompetent recombinant retroviral particles can include any of the activation elements discussed elsewhere herein. In one aspect, provided herein is a replication incompetent recombinant retroviral particle (RIP) that includes a polynucleotide with nucleic acids that encode an anti-idiotype polypeptide provided in any of the aspects and embodiments herein. Such polypeptide typically includes nucleic acids that further encode at least one of a CAR, and LE, an inhibitory RNA, and a cytokine. In some embodiments, the RIP includes a polynucleotide including: A. one or more transcriptional units operatively linked to a promoter active in T cells and/or NK cells, wherein the one or more transcriptional units encode an anti-idiotype polypeptide, and one or more of an engineered T cell receptor or a chimeric antigen receptor (CAR); and B. a pseudotyping element and a T cell activation element on its surface, wherein the T cell activation element is not encoded by a polynucleotide in the replication incompetent recombinant retroviral particle. In some embodiments, the T cell activation element can be any of the activation elements discussed elsewhere herein. In illustrative embodiments, the T cell activation element can be anti-CD3 scFvFc. In another aspect, provided herein is a RIP, including a polynucleotide including one or more transcriptional units operatively linked to a promoter active in T cells and/or NK cells, wherein the one or more transcriptional units encode an anti-idiotype polypeptide and a first signaling polypeptide including an engineered T cell receptor or a chimeric antigen receptor (CAR) and a second signaling polypeptide including a lymphoproliferative element. In some embodiments, the lymphoproliferative element can be a chimeric lymphoproliferative element. In some embodiments of any of the retroviral particle aspects or embodiments provided herein, or any other aspect that includes a retroviral particle, the anti-idiotype polypeptide, engineered T cell receptor, CAR, or other transgene is expressed, displayed, and/or otherwise incorporated in the surface of the replication incompetent retroviral particle at a reduced level that is less than 70%, 60%, 50%, 40%, 30%, 20%, 10%, or 5% of the surface expression compared to when the transgene is expressed from an EF1-a or PGK promoter, and in illustrative embodiments, when the transgene is expressed from an EF1-a or PGK promoter in the absence of additional elements (such as degrons or inhibitory RNAs) to reduce such surface expression. In illustrative embodiments of any of the polynucleotide vector (e.g. RIP) aspects provided herein, or any other aspect that includes a gene vector, the gene vector is substantially free of the protein transcript encoded by nucleic acid of the gene vector, and/or the RIPs do not express or comprise a detectable amount of the engineered T cell receptor or CAR on their surface, or express or comprise a reduced amount of the engineered T cell receptor or CAR on their surface.

Various elements and combinations of elements that are included in RIPs are provided throughout this disclosure, such as, for example, pseudotyping elements, activation elements, and membrane bound cytokines, as well as nucleic acid sequences that are included in a genome of a replication incompetent, recombinant retroviral particle such as, but not limited to, nucleic acid sequences encoding an anti-idiotype polypeptide, nucleic acid sequences encoding a CAR; nucleic acid sequences encoding a lymphoproliferative element; nucleic acids encoding a cytokine; nucleic acid sequences encoding a control element, such as a riboswitch; a promoter, especially a promoter that is constitutively active or inducible in a T cell; and nucleic acids encoding an inhibitory RNA molecule. Furthermore, various aspects provided herein, such as methods of making recombinant retroviral particles, methods for performing adoptive cell therapy, and methods for transducing T cells, produce and/or include replication incompetent, recombinant retroviral particles. Replication incompetent recombinant retroviruses that are produced and/or included in such methods themselves form separate aspects of the present invention as replication incompetent, recombinant retroviral particle compositions, which can be in an isolated form. Such compositions can be in dried down (e.g., lyophilized) form or can be in a suitable solution or medium known in the art for storage and use of retroviral particles.

Recombinant retroviral particle embodiments herein include those wherein the retroviral particle comprises a genome that includes one or more nucleic acids encoding an anti-idiotype polypeptide and one or more inhibitory RNA molecules. Various alternative embodiments of such nucleic acids that encode inhibitory RNA molecules that can be included in a genome of a retroviral particle, including combinations of such nucleic acids with other nucleic acids that encode a CAR or a lymphoproliferative element other than an inhibitory RNA molecule, are included for example, in the inhibitory RNA section provided herein, as well as in various other paragraphs that combine these embodiments. Furthermore, various alternatives of such replication incompetent recombinant retroviruses can be identified by exemplary nucleic acids that are disclosed within packaging cell line aspects disclosed herein. A skilled artisan will recognize that disclosure in this section of a recombinant retroviral particle that includes a genome that encodes one or more (e.g., two or more) inhibitory RNA molecules, can be combined with various alternatives for such nucleic acids encoding inhibitory RNA molecules provided in other sections herein. Furthermore, a skilled artisan will recognize that such nucleic acids encoding one or more inhibitory RNA molecules can be combined with various other functional nucleic acid elements provided herein, as for example, disclosed in the section herein that focuses on inhibitory RNA molecules and nucleic acid encoding these molecules. In addition, the various embodiments of specific inhibitory RNA molecules provided herein in other sections can be used in recombinant retroviral particle aspects of the present disclosure.

Necessary elements of recombinant retroviral vectors, such as lentiviral vectors, are known in the art. These elements are included in the packaging cell line section and in details for making replication incompetent, recombinant retroviral particles provided in the Examples section and as illustrated in WO2019/055946. For example, lentiviral particles typically include packaging elements REV, GAG and POL, which can be delivered to packaging cell lines via one or more packaging plasmids, a pseudotyping element, various examples which are provided herein, which can be delivered to a packaging cell line via a pseudotyping plasmid, and a genome, which is produced by a polynucleotide that is delivered to a host cell via a transfer plasmid. This polynucleotide typically includes the viral LTRs and a psi packaging signal. The 5′ LTR can be a chimeric 5′ LTR fused to a heterologous promoter, which includes 5′ LTRs that are not dependent on Tat transactivation. The transfer plasmid can be self-inactivating, for example, by removing a U3 region of the 3′ LTR. In some non-limiting embodiments, Vpu, such as a polypeptide comprising Vpu (sometimes called a “Vpu polypeptide” herein) including but not limited to, Src-FLAG-Vpu, is packaged within the retroviral particle for any composition or method aspect and embodiment provided herein that includes a retroviral particle. In some non-limiting embodiments, Vpx, such as Src-FLAG-Vpx, is packaged within the retroviral particle. Not to be limited by theory, upon transduction of a T cells, Vpx enters the cytosol of the cells and promotes the degradation of SAMHD1, resulting in an increased pool of cytoplasmic dNTPs available for reverse transcription. In some non-limiting embodiments, Vpu and Vpx is packaged within the retroviral particle for any composition or method aspect and embodiment that includes a retroviral particle provided herein.

Retroviral particles (e.g., lentiviral particles) included in various aspects of the present invention are in illustrative embodiments, replication incompetent, especially for safety reasons for embodiments that include introducing cells transduced with such retroviral particles into a subject. When replication incompetent retroviral particles are used to transduce a cell, retroviral particles are not produced from the transduced cell. Modifications to the retroviral genome are known in the art to assure that retroviral particles that include the genome are replication incompetent. However, it will be understood that in some embodiments for any of the aspects provided herein, replication competent recombinant retroviral particles can be used.

A skilled artisan will recognize that the functional elements discussed herein can be delivered to packaging cells and/or to T cells using different types of vectors, such as expression vectors. Illustrative aspects of the invention utilize retroviral vectors, and in some particularly illustrative embodiments lentiviral vectors. Other suitable expression vectors can be used to achieve certain embodiments herein. Such expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90: 10613-10617); SV40; herpes simplex virus; or a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus), for example a gamma retrovirus; or human immunodeficiency virus (see, e.g., Miyoshi et al., PNAS 94:10319 23, 1997; Takahashi et al., J Virol 73:7812 7816, 1999); and the like.

As disclosed herein, replication incompetent recombinant retroviral particles are a common tool for gene delivery (Miller, Nature (1992) 357:455-460). The ability of replication incompetent recombinant retroviral particles to deliver an unrearranged nucleic acid sequence into a broad range of rodent, primate and human somatic cells makes replication incompetent recombinant retroviral particles well suited for transferring genes to a cell. In some embodiments, the replication incompetent recombinant retroviral particles can be derived from the Alpharetrovirus genus, the Betaretrovirus genus, the Gammaretrovirus genus, the Deltaretrovirus genus, the Epsilonretrovirus genus, the Lentivirus genus, or the Spumavirus genus. There are many retroviruses suitable for use in the methods disclosed herein. For example, murine leukemia virus (MLV), human immunodeficiency virus (HIV), equine infectious anaemia virus (EIAV), mouse mammary tumor virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) can be used. A detailed list of retroviruses may be found in Coffin et al (“Retroviruses” 1997 Cold Spring Harbor Laboratory Press Eds: J M Coffin, S M Hughes, H E Varmus pp 758-763). Details on the genomic structure of some retroviruses may be found in the art. By way of example, details on HIV may be found from the NCBI Genbank (i.e., Genome Accession No. AF033819).

In illustrative embodiments, the replication incompetent recombinant retroviral particles can be derived from the Lentivirus genus. In some embodiments, the replication incompetent recombinant retroviral particles can be derived from HIV, SIV, or FIV. In further illustrative embodiments, the replication incompetent recombinant retroviral particles can be derived from the human immunodeficiency virus (HIV) in the Lentivirus genus. Lentiviruses are complex retroviruses which, in addition to the common retroviral genes gag, pol and env, contain other genes with regulatory or structural function. The higher complexity enables the lentivirus to modulate the life cycle thereof, as in the course of latent infection. A typical lentivirus is the human immunodeficiency virus (HIV), the etiologic agent of AIDS. in vivo, HIV can infect terminally differentiated cells that rarely divide, such as lymphocytes and macrophages.

In illustrative embodiments, replication incompetent recombinant retroviral particles provided herein contain Vpx polypeptide.

In some embodiments, replication incompetent recombinant retroviral particles provided herein comprise and/or contain Vpu polypeptide.

In illustrative embodiments, a retroviral particle is a lentiviral particle. Such retroviral particle typically includes a retroviral genome within a capsid which is located within a viral envelope.

In some embodiments, DNA-containing viral particles are utilized instead of recombinant retroviral particles. Such viral particles can be adenoviruses, adeno-associated viruses, herpesviruses, cytomegaloviruses, poxviruses, avipox viruses, influenza viruses, vesicular stomatitis virus (VSV), or Sindbis virus. A skilled artisan will appreciate how to modify the methods disclosed herein for use with different viruses and retroviruses, or retroviral particles. Where viral particles are used that include a DNA genome, a skilled artisan will appreciate that functional units can be included in such genomes to induce integration of all or a portion of the DNA genome of the viral particle into the genome of a T cell transduced with such virus.

In some embodiments, the HIV RREs and the polynucleotide region encoding HIV Rev can be replaced with N-terminal RGG box RNA binding motifs and a polynucleotide region encoding ICP27. In some embodiments, the polynucleotide region encoding HIV Rev can be replaced with one or more polynucleotide regions encoding adenovirus E1B 55-kDa and E4 Orf6.

In certain aspects, replication incompetent recombinant retroviral particles can include nucleic acids encoding a self-driving CAR, as disclosed elsewhere herein. As a non-limiting example, such embodiments are retroviral particles whose genome comprises one or more first transcriptional units operably linked to an inducible promoter inducible in at least one of a T cell or an NK cell, and one or more second transcriptional units operably linked to a constitutive T cell or NK cell promoter, wherein the number of nucleotides between the 5′ end of the one or more first transcriptional units and the 5′ end of the one or more second transcriptional units is less than the number of nucleotides between the 3′ end of the one or more first transcriptional units and the 3′ end of the one or more second transcriptional units,
a. wherein at least one of the one or more first transcriptional units encodes a lymphoproliferative element,
b. and wherein at least one of the one or more second transcriptional units encodes a first chimeric antigen receptor (CAR), wherein the CAR comprises an antigen-specific targeting region (ASTR), a transmembrane domain, and an intracellular activating domain, and wherein nucleic acids within the first transcriptional unit or the second transcriptional unit encode an anti-idiotype polypeptide according to an of the embodiments provided herein.

In some embodiments, the replication incompetent recombinant retroviral particles can further display a T cell activation element.

Not to be limited by theory, T cells contacted and transduced with these replication incompetent recombinant retroviral particles that include nucleic acids encoding a self-driving CAR, can receive an initial boost of transcription from the CAR-stimulated inducible promoters as the T cell activation element can stimulate the inducing signal of the CAR-stimulated inducible promoters. The binding of the T cell activation element can induce the calcium ion influx that results in dephosphorylation of NFAT and its subsequent nuclear translocation and binding to NFAT-responsive promoters. The lymphoproliferative elements transcribed and translated from these CAR-stimulated inducible promoters can give an initial increase in proliferation to these cells. In illustrative embodiments, the T cell activation element can be a membrane-bound anti-CD3 antibody, and can be GPI-linked or otherwise displayed on virus. In some embodiments, the membrane-bound anti-CD3 antibody can be fused to a viral envelope protein, such as MuLV, VSV-G, a Henipavirus-G such as NiV-G, or variants and fragments thereof.

In some embodiments, the isolated replication incompetent retroviral particles are a large-scale batch contained in a large-scale container. Such large-scale batch can have titers, for example of 10⁶ - 10⁸ TU/ml and a total batch size of between 1×10¹⁰ TU and 1×10¹³ TU, 1×10¹¹ TU and 1×10¹³ TU, lx10¹² TU and 1×10¹³ TU, 1×10¹⁰ TU and 5×10¹² TU, or 1×10¹¹ TU and 5×10¹² TU. In illustrative embodiments, retroviral particles for any aspect or embodiment provided herein are substantially pure, as discussed in more detail herein.

### Retroviral Genome Size

In the methods and compositions provided herein, the recombinant retroviral genomes, in non-limiting illustrative examples, lentiviral genomes, have a limitation to the number of polynucleotides that can be packaged into the viral particle. In some embodiments provided herein, the polypeptides encoded by the polynucleotide encoding region can be truncations or other deletions that retain a functional activity such that the polynucleotide encoding region is encoded by less nucleotides than the polynucleotide encoding region for the wild-type polypeptide. In some embodiments, the polypeptides encoded by the polynucleotide encoding region can be fusion polypeptides that can be expressed from one promoter. In some embodiments, the fusion polypeptide can have a cleavage signal to generate two or more functional polypeptides from one fusion polypeptide and one promoter. Furthermore, some functions that are not required after initial ex vivo transduction are not included in the retroviral genome, but rather are present on the surface of the replication incompetent recombinant retroviral particles via the packaging cell membrane. These various strategies are used herein to maximize the functional elements that are packaged within the replication incompetent recombinant retroviral particles. In some embodiments, the recombinant retroviral genome to be packaged can be between 1,000, 2,000, 3,000, 4,000, 5,000, 6,000, 7,000, and 8,000 nucleotides on the low end of the range and 2,000, 3,000, 4,000, 5,000, 6,000, 7,000, 8,000, 9,000, 10,000, and 11,000 nucleotides on the high end of the range. The retroviral genome to be packaged includes one or more polynucleotide regions encoding a first and second engineering signaling polypeptide as disclosed in detail herein. In some embodiments, the recombinant retroviral genome to be packaged can be less than 5,000, 6,000, 7,000, 8,000, 9,000, 10,000, or 11,000 nucleotides. Functions discussed elsewhere herein that can be packaged include required retroviral sequences for retroviral assembly and packaging, such as a retroviral rev, gag, and pol coding regions, as well as a 5′ LTR and a 3′ LTR, or an active truncated fragment thereof, a nucleic acid sequence encoding a retroviral cis-acting RNA packaging element, and a cPPT/CTS element. Furthermore, in illustrative embodiments a replication incompetent recombinant retroviral particle herein can include any one or more or all of the following, in some embodiments in reverse orientation with respect to a 5′ to 3′ orientation established by the retroviral 5′ LTR and 3′ LTR (as illustrated in WO2019/055946 as a non-limiting example): one or more polynucleotide regions encoding a first and second engineering signaling polypeptide, at least one of which includes at least one lymphoproliferative element; a second engineered signaling polypeptide that can include a chimeric antigen receptor; an miRNA, a control element, such as a riboswitch, which typically regulates expression of the first and/or the second engineering signaling polypeptide; a safety switch polypeptide, an intron, a promoter that is active in a target cell, such as a T cell, a 2A cleavage signal and/or an IRES.

### Kits and Commercial Products

In another aspect, provided herein is a delivery composition or suspension, for example for treating or preventing a disease, for example cancer or tumor growth, comprising polynucleotides, such as polynucleotide vectors, in illustrative replication incompetent recombinant retroviral particle (RIPs), or modified cells, such as a modified lymphocytes, modified TILs, modified lymphocytes other than B cells, or modified T cells and/or modified NK cells as an active ingredient. In another aspect, provided herein is an infusion composition or other cell formulation for treating or preventing cancer or tumor growth comprising polynucleotides such as polynucleotide vectors, in illustrative embodiments RIPs, or modified cells, such as a modified lymphocytes, modified TILs, modified lymphocytes other than B cells, or modified T cells and/or modified NK cells. The polynucleotides such as polynucleotide vectors, in illustrative embodiments RIPs, or modified cells, such as a modified lymphocytes, modified TILs, modified lymphocytes other than B cells, or modified T cells and/or modified NK cells of the delivery composition or infusion composition can include any of the aspects, embodiments, or subembodiments discussed above or elsewhere herein, for example that include nucleic acids that encode anti-idiotype polypeptides, as well as a CAR, an LE, a cytokine and/or a TCR.

Provided herein in one aspect is a container, such as a commercial container or package, or a kit comprising the same, comprising polynucleotides, such as polynucleotide vectors, for example RIPs, or modified cells, such as a modified lymphocytes, modified TILs, modified lymphocytes other than B cells, or modified T cells and/or modified NK cells according to any of the aspects and embodiments provided herein. As a non-limiting example, the polynucleotides, such as polynucleotide vectors, for example RIPs, or modified cells, such as a modified lymphocytes, modified TILs, modified lymphocytes other than B cells, or modified T cells and/or modified NK cells can comprise in their genome a polynucleotide comprising one or more nucleic acid sequences operatively linked to a promoter active in T cells and/or NK cells. In some embodiments, a nucleic acid sequence of the one or more nucleic acid sequences can encode an anti-idiotype polypeptide, an inhibitory RNA, a cytokine, a lymphoproliferative element and/or a chimeric antigen receptor (CAR) comprising an antigen-specific targeting region (ASTR), a transmembrane domain, and an intracellular activating domain. In some embodiments, a nucleic acid sequence of the one or more nucleic acid sequences can encode one, two or more inhibitory RNA molecules directed against one or more RNA targets.

The container that contains the polynucleotides, such as polynucleotide vectors, for example RIPs, or modified cells, such as a modified lymphocytes, modified TILs, modified lymphocytes other than B cells, or modified T cells and/or modified NK cells in any aspect or embodiment, including commercial container as well as kits, can be a cryopreservation infusion bag, tube, vial, well of a plate, or other vessel for storage of polynucleotides, such as polynucleotide vectors, for example RIPs, or modified cells, such as a modified lymphocytes, modified TILs, modified lymphocytes other than B cells, or modified T cells and/or modified NK cells. In fact, some aspects provided herein, comprise a container comprising polynucleotides, such as polynucleotide vectors, for example RIPs, or modified cells, such as a modified lymphocytes, modified TILs, modified lymphocytes other than B cells, or modified T cells and/or modified NK cells, wherein such biological include any nucleic acid(s) or other component(s) disclosed herein. Such container in illustrative embodiments includes substantially pure polynucleotides, such as polynucleotide vectors, for example RIPs, or modified cells, such as a modified lymphocytes, modified TILs, modified lymphocytes other than B cells, or modified T cells and/or modified NK cells, sometimes referred to herein for shorthand, as substantially pure biologic. Typically, a preparation and/or container of substantially pure biologic is sterile, and negative for mycoplasma, replication competent retroviruses, and adventitious viruses according to standard protocols (see e.g., “Viral Vector Characterization: A Look at Analytical Tools”; Oct. 10, 2018 (available at https://cellculturedish.com/viral-vector-characterization-analytical-tools/)). Exemplary methods for generating substantially pure biologics and cells for cell therapy are known. For example, for such methods with respect to RIPs, viral supernatants can be purified by a combination of depth filtration, TFF, benzonase treatment, diafiltration, and formulation. In certain illustrative embodiments, a substantially pure biologic meets all of the following characteristics based on quality control testing results:
a) negative for mycoplasma;
b) endotoxin at less than 25 EU/ml, and in certain further illustrative embodiments, less than 10 EU/ml;
c) absence of replication competent retroviruses detected of the same type as purposefully in the container (e.g., lentiviruses) detected; and
d) absence of adventitious viruses detected;

Furthermore, if the biologic is a viral particle, such as a retroviral particle, in exemplary embodiments, it meets the following quality control testing results:
a) less than 1 pg host cell DNA/ viral TU, and in certain further illustrative embodiments, less than 0.3 pg / TU;
b) less than 100 residual plasmid copies/ viral TU, and in certain further illustrative embodiments, less than 10 copies/viral TU of any plasmid used to make the recombinant retroviral particles;
c) less than 1 ng HEK protein/ TU, and in certain further illustrative embodiments, less than 50 pg HEK protein /TU; and
d) greater than 100 TU / ng P24 protein, and in certain further illustrative embodiments, greater than 10,000 TU /ng P24 protein.

Retroviral particles are typically tested against release specifications that include some or all of those provided above, before they are released to a customer. Potency of each particle may be defined on the basis of p24 viral capsid protein by ELISA, viral RNA genome copies by q-RT PCR, measurement of reverse transcriptase activity by qPCR-based product-enhanced RT (PERT) assay but can all be converted to infectious titer by measuring functional gene transfer Transducing Units (TUs) in a bioassay.

Determination of infectious titer of purified bulk retrovirus material and finished product by bioassay and qPCR is an exemplary analytical test method for the determination of infectious titer of retroviruses. An indicator cell bank (such as F1XT) may be grown for example in serum free media, seeded at 150,000 cells per well, followed by exposure to serial dilutions of the retrovirus product. Dilutions of purified retrovirus particles are made on indicator cells, for example from 1:200 to 1:1,600. A reference standard virus may be added for system suitability. Following 4 days of incubation with retrovirus, the cells are harvested, DNA extracted and purified. A standard curve, for example from 100-10,000,000 copies/ well, of human genome and unique retroviral genome sequence plasmid pDNA amplicons are used followed by addition of genomic DNA of the cell samples exposed to retrovirus particles. For each PCR reaction, the Cq values of both the retrovirus amplicon and the endogenous control such as humanRNAseP are extrapolated back to copies per reaction. From these values the integrated genome copy number is calculated. In some cases, indicator cells such as 293T have been characterized as being triploid, hence 3 copies of a single copy gene per cell should be utilized in the calculation. Using the initial viable cell count per well, the volume of retrovirus added to the cells and the genome copy number ratio a Transducing Unit (TU) per ml retrovirus particles may be determined.

Potency testing can include potency testing against release specifications with purity and specific activity. For example, potency release testing of final product can include measurement of the number of Transducing Units (TU) can be compared to viral particle quantity (e.g., by performing an ELISA against a viral protein, for example for lentivirus by performing a p24 capsid protein ELISA with a cutoff of at least 100, 1,000, 2,000 or 2,500 TU/ng p24), and CAR functionality, for example by measuring interferon gamma release by a reporter cell line exposed to gene modified cells.

In any of the kit or isolated replication incompetent recombinant retroviral particle aspects herein, that include a container of such retroviral particles, sufficient recombinant retroviral particles are present in the container to achieve an MOI (the number of Transducing Units, or TUs applied per cell) in a reaction mixture made using the retroviral particles, of between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15 or at least 0.1, 0.5, 1, 2, 2.5, 3, 5, 10 or 15, or to achieve an MOI of at least 0.1, 0.5, 1, 2, 2.5, 3, 5, 10 or 15. The Transducing Units of virus particles provided in the kit should enable the use an MOI that prevents producing too many integrants in an individual cell, on average less than 3 lentigenome copies per cellular genome and more preferably 1 copy per cell. For kit and isolated retroviral particle embodiments, such MOI can be based on 1, 2.5, 5, 10, 20, 25, 50, 100, 250, 500, or 1,000 ml of reaction mixture assuming 1×10° target cells/ml, for example in the case of whole blood, assuming 1 × 10⁶ PBMCs/ml of blood. Accordingly, a container of retroviral particles can include between 1 × 10⁵ and 1 × 10⁹, 1 × 10⁵ and 1 × 10^{8,} 1 × 10⁵ and 5 × 10⁷, 1 × 10⁵ and 1 × 10⁷, 1 × 10⁵ and 1 × 10⁶; 5 × 10⁵ and 1 × 10⁹; 5 × 10⁵ and 1 × 10^{8,} 5 × 10⁵ and 5 × 10⁷, 5 × 10⁵ and 1 × 10⁷, 5 × 10⁵ and 1 × 10⁶, or 1 × 10⁷ and 1 × 10⁹, 1 × 10⁷ and 5 × 10⁷, 1 × 10⁶ and 1 × 10⁷, and 1 × 10⁶ and 5 × 10⁶ TUs. In certain illustrative embodiments, the container can contain between 1 × 10⁷ and 1 × 10⁹, 5 × 10⁶ and 1 × 10^{8,} 1 × 10⁶ and 5 × 10⁷, 1 × 10⁶ and 5 × 10⁶ or between 5 ×10⁷ and 1 ×10⁸ retroviral Transducing Units. Not to be limited by theory, such numbers of particles would support between 1 and 100 ml of blood at an MOI of between 1 and 10. In some illustrative embodiments, as indicate herein, as little as 10 ml, 5 ml, 3 ml, or even 2.5 ml of blood can be processed for T cell and/or NK cell modification and optionally subcutaneous and/or intramuscular administration methods provided herein. Thus, an advantage of the present methods is that in some illustrative embodiments, they require far fewer retroviral particle Transducing Units than prior methods that involve nucleic acids encoding a CAR, such as CAR-T methods.

Each container that contains retroviral particles, can have, for example, a volume of between 0.05 ml and 5 ml, 0.05 ml and 1 ml, 0.05 ml and 0.5 ml, 0.1 ml and 5 ml, 0.1 ml and 1 ml, 0.1 ml and 0.5 ml, 0.1 and 10 ml, 0.5 and 10 ml, 0.5 ml and 5 ml, 0.5 ml and 1 ml, 1.0 ml and 10.0 ml, 1.0 ml and 5.0 ml, 10 ml and 100 ml, 1 ml and 20 ml, 1 ml and 10 ml, 1 ml and 5 ml, 1 ml and 2 ml, 2 ml and 20 ml, 2 ml and 10 ml, 2 ml and 5 ml, 0.25 ml to 10 ml, 0.25 to 5 ml, or 0.25 to 2 ml.

In some embodiments where the kit includes modified cells, such as modified lymphocytes, modified TILs, modified lymphocytes that are not B cells, such as modified T cells and/or modified NK cells in a cell suspension within a commercial container, for example a cryopreservation infusion bag, the container, such as the cryopreservation infusion bag, can hold 5, 10, 15, 20, 25, 50, 75, 100, 150, 200, 250, 300, 400, or 500 ml or less of blood. In some embodiments, the container, for example cryopreservation infusion bag can hold at least 5, 10, 15, 20, 25, 50, 75, 100, 150, 200, 250, 300, 400, or 500 ml of blood. In some embodiments, the container, for example cryopreservation infusion bag can hold between 1, 2, 3, 4, 5, 10, 15, 20, 25, and 50 ml of blood on the low end of the range and 10, 15, 20, 25, 50, 75, 100, 150, 200, 250, 300, 400, and 500 ml of blood on the high end of the range. In some embodiments, the container, for example cryopreservation infusion bagcan hold between 1, 2, 3, 4, 5, 10, 15, 20, 25, and 50 ml of blood on the low end of the range and 10, 15, 20, 25, 50, 75, 100, 150, 200, 250, 300, 400, and 500 ml of blood on the high end of the range. For example, the container, for example cryopreservation infusion bag can hold between 1 and 10 ml, 5 and 25 ml, 10 and 50 ml, 25 and 100 ml, 50 and 200 ml, or 100 and 500 ml of blood. In some embodiments, the container, for example cryopreservation infusion bag can include heparin. In other embodiments, the container, for example cryopreservation infusion bag do not include heparin.

In some embodiments where the kit includes modified cells, such as a modified lymphocytes, modified TILs, modified lymphocytes other than B cells, or modified T cells and/or modified NK cells in a cell suspension within a commercial container, such as a cell cryopreservation infusion bag, the number of cells delivered can be sufficient to provide between 1 × 10⁵ cells and 1 × 10⁹, between 1 × 10⁶ cells and 1 × 10⁹, or between 1 × 10⁶ cells and 5 × 10^{8,} for example CAR-positive viable T cells and/or NK cells per kg of body weight of the subject to which the cells are to be delivered. Accordingly, in some embodiments the commercial container, can include the aforementioned ranges × 50-150 kg, or 50-100 kg. In some embodiments, the commercial container includes between 1 × 10⁷ and 1 × 10¹¹ cells, between 1 × 10⁸ and 1 × 10¹¹ cells, or between 1 × 10⁸ and 5 × 10¹⁰ cells, for example CAR-positive viable T cells and/or NK cells, or in an illustrative embodiment, cells that are positive for an anti-idiotype extracellular recognition domain.

In illustrative embodiments, the polynucleotides encoding the anti-idiotype polypeptide and in illustrative embodiments the CAR are located in the genome of retroviral particles, typically substantially pure retroviral particles, according to any of the replication incompetent recombinant retroviral particle aspects and embodiments provided herein. In illustrative embodiments, the replication incompetent recombinant retroviral particles in the kit comprise a polynucleotide comprising one or more transcriptional units operatively linked to a promoter active in T cells and/or NK cells, wherein the one or more first transcriptional units encode a first polypeptide comprising an anti-idiotype polynucleotide and a CAR or an LE and optionally encode a second polypeptide comprising the other of a CAR or an LE, according to any of the embodiments provided herein.

A kit provided herein can include a container containing the polynucleotides, such as polynucleotide vectors, for example RIPs, or modified cells, such as, modified lymphocytes, modified TILs, modified lymphocytes other than B cells, for example modified T cells and/or NK cells, and an accessory kit. The accessory kit components can include one or more of the following:
a) one or more containers containing a delivery solution compatible with, in illustrative embodiments effective for, and in further illustrative embodiments adapted for subcutaneous and/or intramuscular administration as provided herein;
b) one or more containers of hyaluronidase as provided herein;
c) one or more blood bags such as a blood collection bag, in illustrative embodiments comprising an anticoagulant in the bag, or in a separate container, a blood processing buffer bag, a blood processing waste collection bag, and a blood processing cell sample collection bag;
d) one or more sterile syringes compatible with, in illustrative embodiments effective for, and in further illustrative embodiments adapted for, subcutaneous or intramuscular delivery of T cells and/or NK cells;
e) a T cell activation element as disclosed in detail herein, for example anti-CD3 provided in solution in the container containing the retroviral particle, or in a separate container, or in illustrative embodiments, is associated with a surface of the replication incompetent retroviral particle;
f) one or a plurality of leukoreduction filtration assemblies;
g) one or more containers containing a solution or media compatible with, in illustrative embodiments effective for, and in further illustrative embodiments adapted for transduction of T cells and/or NK cells;
h) one or more containers containing a solution or media compatible with, in illustrative embodiments effective for, and/or in further illustrative embodiments adapted for rinsing T cells and/or NK cells;
i) one or more containers containing a pH-modulating pharmacologic agent;
j) one or more containers containing polynucleotides, typically substantially pure polynucleotides (e.g. found within recombinant retroviral particles according to any embodiment herein), comprising one or more second transcriptional units operatively linked to a promoter active in T cells and/or NK cells, wherein the one or more second transcriptional units encode a polypeptide comprising a second CAR directed against a different target epitope, and in certain embodiments a different antigen, in illustrative embodiments found on a same target cancer cell (e.g. B cell);
k) one or more containers containing a cognate antigen for the first CAR and/or the second CAR encoded by the nucleic acids (e.g., retroviral particles); and
l) instructions, either physically or digitally associated with other kit components, for the use thereof, for example for modifying T cells and/or NK cells, for delivering modified T cells and/or NK cells to a subject subcutaneously or intramuscularly, and/or for treating tumor growth or cancer in a subject.

In any of the kit aspects and embodiments herein that include an antigen or a cognate antigen, less than 50%, 40%, 30%, 20%, 10%, 5%, or 1% of the polypeptides in the kit are non-human, i.e., produced from non-human sources.

In some embodiments, the kit may be a single-pack/use kit, but in other embodiments the kit is a multi-pack or multi-use kit for the processing of more than one blood sample from contacting with nucleic acids encoding a CAR optionally thru subcutaneous administration. Typically, a container of nucleic acids encoding a CAR (and optionally a paired container of nucleic acids encoding a second CAR in certain embodiments) in the kit is used for one performance of a method for modifying T cells and/or NK cells and optionally subcutaneous administration. The container(s) containing nucleic acids encoding a CAR and optionally a second CAR is typically stored and shipped frozen. Thus, a kit can include sufficient containers (e.g., vials) of nucleic acids encoding a CAR (and optionally paired containers encoding a second CAR in certain embodiments) for 1, 2, 3, 4, 5, 6, 10, 12, 20, 24, 50 and 100 performances of a method for modifying a T cell and/or NK cell provided herein, and thus can include 1, 2, 3, 4, 5, 6, 10, 12, 20, 24, 50 and 100 containers (e.g., vials) of nucleic acids encoding the CAR (e.g., retroviral particles), and similarly is considered a 1, 2, 3, 4, 5, 6, 10, 12, 20, 24, 50 and 100 pack, performance, administration or X kit, respectively. Similarly, accessory components in the kit would be provided for similar numbers of performances of a method for modifying T cells and/or NK cells and optionally subcutaneous administration, using the kit.

The one or more leukoreduction filtration assemblies, if present in such a kit, typically include(s) one or a plurality of leukoreduction filters or leukoreduction filter sets, each typically within a filter enclosure, as well as a plurality of connected sterile tubes connected or adapted to be connected thereto, and a plurality of valves connected or adapted to be connected thereto, that are adapted for use in a single-use closed blood processing system. Typically, there is one leukoreduction filtration assembly for each container of nucleic acid encoding a CAR in a kit. Thus a 20-pack kit in illustrative embodiments, includes 20 vials of nucleic acids encoding a CAR and 20 leukoreduction filtration assemblies. In some embodiments, a kit herein comprises one or a plurality of containers containing nucleic acids and one or more leukoreduction filtration assemblies. Such a kit can optionally be intended to be used for administration to a subject via any route including for example, infusion or in illustrative embodiments intramuscular and/or in further illustrative embodiments, subcutaneous delivery. Thus, such a kit optionally includes other accessory components that are intended to be used with such route of administration. The one or more containers of subcutaneous or intramuscular delivery solution is discussed in more detail herein, is typically sterile and can include a total combined volume, or individually per container, of 100 ml to 5 L, 1 ml to 1 L, 1 ml to 500 ml, 1 ml to 250 ml, 1 ml to 200 ml, 1 ml to 100 ml, 1 ml to 10 ml, or 1 ml to 5 ml; 5 ml to 1 L, 5 ml to 500 ml, 5 ml to 250 ml, 5 ml to 100 ml, 5 ml to 10 ml, or approximately 5 ml. In some illustrative embodiments, the kit comprises a plurality of containers of subcutaneous delivery solution, with each container having a volume of between 10 ml and 200 ml, 10 ml and 100 ml, 1 ml and 20 ml, 1 ml and 10 ml, 1 ml and 5 ml, 1 ml and 2 ml, 2 ml and 20 ml, 2 ml and 10 ml, 2 ml and 5 ml, 0.25 ml to 10 ml, 0.25 to 5 ml, or 0.25 to 2 ml. In illustrative embodiments, there is one container of delivery solution for each container of nucleic acid encoding a CAR in a kit. Thus, a 20-pack kit in illustrative embodiments, includes 20 vials of nucleic acids encoding a CAR and 20 containers of sterile delivery solution.

In certain kit aspects, provided herein are embodiments in which either or both the container(s) containing nucleic acids encoding a first CAR and optionally nucleic acids encoding a second CAR, are nucleic acids according to any of the self-driving CAR embodiments provided herein. In such embodiments, accessory components of the kit can further include one or more of the following:
a. one or more containers containing a delivery solution adapted for, compatible with, and/or effective for, intravenous or intraperitoneal administration as provided herein; and
b. Instructions, either physically or digitally associated with other kit components, for the use thereof, for example for delivering modified T cells and/or NK cells to a subject intravenously or intraperitoneally.

In certain aspects, provided herein are the use of a RIPs in the manufacture of a kit for modifying a T cell or NK cell, wherein the use of the kit includes: contacting the T cell or NK cell ex vivo with the replication incompetent recombinant retroviral particle, wherein the replication incompetent recombinant retroviral particle includes a pseudotyping element on a surface and a T cell activation element on the surface, wherein said contacting facilitates transduction of the T cell or NK cell by the replication incompetent recombinant retroviral particle, thereby producing a modified and in illustrative embodiments genetically modified T cell or NK cell.

In some aspects, provided herein are aspects that include the use of a RIP in the manufacture of a kit for modifying a T cell or NK cell. Details regarding polynucleotides, and replication incompetent recombinant retroviral particles that contain such polynucleotides are disclosed in more detail herein, and in the Exemplary Embodiments section. In some embodiments, the T cell or NK cell can be from a subject. In some embodiments, the T cell activation element can be membrane-bound. In some embodiments, the contacting can be performed for between 1, 2, 3, 4, 5, 6, 7, or 8 hours on the low end of the range and 4, 5, 6, 7, 8, 10, 12, 15, 18, 21, and 24 hours on the high end of the range, for example, between 1 and 12 hours. The replication incompetent recombinant retroviral particle for use in the manufacture of a kit can include any of the aspects, embodiments, or subembodiments discussed elsewhere herein.

Furthermore, provided herein in another aspect is a container, such as a commercial container or package, or a kit comprising the same, comprising isolated packaging cells, in illustrative embodiments isolated packaging cells from a packaging cell line, according to any of the packaging cell and/or packaging cell line aspects provided herein. In some embodiments, the kit includes additional containers that include additional reagents such as buffers or reagents used in methods provided herein. Furthermore, provided herein in certain aspects are use of any replication incompetent recombinant retroviral particle provided herein in any aspect, in the manufacture of a kit for modifying and in illustrative embodiments genetically modifying a T cell or NK cell according to any aspect provided herein. Furthermore, provided herein in certain aspects are use of any packaging cell or packaging cell line provided herein in any aspect, in the manufacture of a kit for producing the replication incompetent recombinant retroviral particles according to any aspect provided herein.

### Self-Driving Car Methods and Compositions

Provided herein in certain aspects are polynucleotides referred to herein as “self-driving CARs” that encode a membrane-bound lymphoproliferative element whose expression in a T cell or NK cell is under the control of an inducible promoter that is induced by the binding of an antigen to an extracellular binding pair member polypeptide that is expressed by the T cell or NK cell and is functionally linked to a intracellular activating domain, for example a CD3 zeta intracellular activating domain or any of the intracellular activating domains disclosed elsewhere herein. In illustrative embodiments herein, an anti-idiotype polypeptide is co-expressed by the T cell or NK cell to provide additional functional optionality for self-driving CARs. In an illustrative embodiments, the co-expressed anti-idiotype polypeptide is a safety switch. In illustrative embodiments, such a binding pair member polypeptide is a CAR. In other embodiments, such a binding pair member polypeptide is a TCR. Thus, in certain embodiments, provided herein are polynucleotides that include an inducible promoter operably linked to a nucleic acid encoding a membrane-bound lymphoproliferative element, that is induced by CAR-binding to its target. Expression of the lymphoproliferative element can induce proliferation of the T cell or NK cell. Provided herein in certain aspects are genetically modified or transduced T cells referred to herein as “self-driving CAR-T cells” that include a self-driving CAR. Any of the embodiments that include a self-driving CAR-T cell could include a “self-driving CAR NK cell,” which is a genetically modified or transduced NK cell that includes a self-driving CAR. In some embodiments, the self-driving CAR NK cell is present in addition to the self-driving CAR-T cell. In other embodiments, the self-driving CAR NK cell is present instead of the self-driving CAR-T cell. Various embodiments that include a self-driving CAR are disclosed in the Exemplary Embodiments section herein and can be combined with any of the embodiments or details of this section.

### Illustrative Cell Processing Methods for Genetically Modifying T Cells and/or NK Cells

Methods provided herein in illustrative aspects include methods for modifying T cells and/or NK cells, or related methods of making cell formulations, that include contacting blood cells comprising lymphocytes (e.g., NK cells and/or T cells) ex vivo in a reaction mixture, with recombinant vectors such as replication incompetent recombinant retroviral particles, that are or include polynucleotides that encode a CAR. In illustrative embodiments, the reaction mixture includes a T cell activation element, either in solution or on the surface of the recombinant retroviral particles, to facilitate genetic modification of T cells in the reaction mixtureSome of the methods provided herein include an optional step where blood is collected (110) from a subject. Blood can be collected or obtained from a subject by any suitable method known in the art as discussed in more detail herein. For example, the blood can be collected by venipuncture, apheresis or any other blood collection method by which a sample of blood is collected. In some embodiments, the volume of blood collected is between 1 and 120 ml. In illustrative embodiments, especially those in which a subject from whom blood is taken has normal levels of NK cells and in illustrative embodiments, T cells, the volume of blood collected is between 1 ml and 25 ml.

It is noteworthy that some embodiments of methods for modifying and in illustrative embodiments genetically modifying provided herein do not include a step of collecting blood from a subject. Regardless of whether blood is collected from a subject, in illustrative method aspects provided herein for modifying lymphocytes (e.g., T cells and/or NK cells), the lymphocytes are contacted with encapsulated nucleic acid vectors (e.g. replication incompetent retroviral particles) in a reaction mixture. In illustrative embodiments, this contacting, and the reaction mixture in which the contacting occurs, takes place within a closed cell processing system, as discussed in more detail herein. Such a closed processing system and method used in some aspects and embodiments of systems and methods provided herein can be any system and method known in the art. As non-limiting examples, the system or method can be a traditional closed cell processing system and method, or a system or method referred to herein as a “more recent” method or system (See e.g., WO2018/136566 and WO2019/055946, each incorporated herein by reference in their entirety). In traditional closed cell processing methods that involve genetic modification and/or transductions of lymphocytes ex vivo, especially in methods for autologous cell therapy, many steps occur over days, such as PBMC enrichment(s), washing(s), cell activation, transduction, expansion, collection, and optionally reintroduction. In more recent methods, some of the steps and time involved in this ex vivo cell processing have been reduced (see, e.g., WO2018/136566). In other more recent methods (See FIG. 1A), some of the steps and time involved in this ex vivo cell processing have been further reduced or as with, for example, the ex vivo expansion step, eliminated (see, e.g., WO2019/055946). These more recent methods (as well as the further improved cell processing methods provided herein), furthermore use a rapid ex vivo transduction process, for example that includes no or minimal preactivation (e.g., less than 30, 15, 10, or 5 minutes of contacting lymphocytes such as T cells and/or NK cells with an activation agent before they are contacted with retroviral particles). In certain embodiments of such methods, a T cell and/or NK cell activation element is present in the reaction mixture in which the contacting step occurs. In illustrative embodiments, the T cell and/or NK cell activation element is associated with surfaces of retroviral particles present in the reaction mixture. In illustrative embodiments, such a method that uses rapid ex vivo gene modification without an ex vivo expansion step is used in a rapid point-of-care (rPOC) autologous cell therapy method. However, such more recent methods still involve a PBMC enrichment step/procedure (120A), which typically takes at least around 1 hour within the closed system, followed by cell counting, transfer and media addition, which takes at least around 45 additional minutes before lymphocytes are contacted with retroviral particles to form a transduction reaction mixture (130A). Following the “viral transduction” step, which typically is a contacting step with incubating as discussed in detail herein, lymphocytes are typically washed away from retroviral particles that remain in suspension (140A), for example using a Sepax, and collected by resuspending the PBMCs in a delivery solution (150A) to form a cell formulation typically in an infusion bag for reinfusion, a syringe for injection, or cryopreservation vial for storage (160A). As discussed in further detail herein, traditional PBMC enrichment procedures typically involve ficoll density gradients and centrifugal (e.g., centrifugation) or centripetal (e.g., Sepax) forces or use leukophoresis to enrich PBMCs.

The inventors have observed that subcutaneous administration has shown surprising results, with increased engraftment of modified and/or genetically modified lymphocytes relative to modified and/or genetically modified lymphocytes introduced through intravenous infusion. This has led to more effective CAR-dependent tumor reduction and elimination in animals. In illustrative embodiments, modified lymphocytes (e.g., T cells and/or NK cells) in a solution are introduced, and in illustrative embodiments reintroduced into a subject by subcutaneous administration, delivery, or injection. In some examples of these embodiments that involve contacting lymphocytes in reaction mixtures with retroviral particles such as those exemplified in FIG. 1, including illustrative embodiments that include at least some other blood components not typically present after the lymphocytes are isolated in a PBMC enrichment procedure, resulting cell formulations, which are separate aspects provided herein, are optionally administered (e.g., readministered) into a subject. In illustrative embodiments, (FIG. 1D) where a PBMC enrichment procedure is not used after lymphocytes are contacted with retroviral particles, cell formulations produced there can be reintroduced back into a subject using subcutaneous or intramuscular administration. Thus, as discussed in more detail herein, some aspects provided herein are cell formulations, as well as delivery solutions (i.e., excipients) for making such cell formulations, that are compatible with, in illustrative embodiments effective for, and in further illustrative embodiments adapted for subcutaneous delivery. Not to be limited by theory, it is believed that the presence of additional blood cells, especially neutrophils, in a process that only uses cell processing filters to concentrate and/or wash lymphocytes, such as HemaTrate filters, renders the cell preparations more amenable to subcutaneous delivery to avoid some additional risks present if these other blood cell types, especially neutrophils or aggregated T cells, are infused directly back into the blood of a patient. For example, a subcutaneous formulation of retrovirus reconstituted with total nucleated cells on a lymphoreduction filter may contain, in addition to lymphocytes, neutrophils (or more generally granulocytes). In illustrative embodiments, the cell formulation comprises neutrophils, B cells, monocytes, red blood cells, basophils, eosinophils, and/or macrophages together with modified T cells (CAR-T cells) and/or NK cells (CAR-NK cells). A subcutaneous or intramuscular formulation and administration are advantageous over intravenous formulation and administration because a formulation (suspension) of retrovirus reconstituted with lymphocytes may further comprise cellular aggregates and express adhesion receptors that may introduce pulmonary congestion with intravenous delivery.

Methods for subcutaneous administration are well known in the art and typically involve administration into the fat layer under the skin. It should be noted that it is contemplated that any embodiment herein that involves subcutaneous delivery, can instead be intramuscular delivery, which is delivery into the muscle, intradermal, or intratumoral delivery. In some embodiments, subcutaneous administrations can be performed in the upper thigh, upper arm, abdomen, or upper buttocks of a subject. Subcutaneous administration is distinguishable from intraperitoneal administration, which penetrates through the fatty layer used in subcutaneous administration and delivers a formulation or drug into the peritoneum of the subject.

### Steps and Reaction Mixtures for Methods Herein

Provided herein in certain aspects, is a method of administering modified cells to a subject, which can include before delivering the modified cells to the subject, a step of transducing, transfecting, genetically modifying, and/or modifying the cells. The cells can be lymphocytes, such as a (typically a population of) peripheral blood mononuclear cells (PBMCs), typically T cells and/or an NK cells, and in certain illustrative embodiments resting T cells and/or resting NK cells. The transducing, transfecting, modifying and/or genetically modifying step, can include contacting the lymphocytes with a population of recombinant nucleic acid/polynucleotide vectors, which in certain illustrative embodiments include nucleic acids encoding an anti-idiotype polypeptide, and which in illustrative embodiments are replication incompetent recombinant retroviral particle (RIPs), wherein said contacting (and incubation under contacting conditions) facilitates membrane association, membrane fusion or endocytosis, and optionally transduction or transfection of the cells, for example resting T cells and/or NK cells by the recombinant nucleic acid vectors, thereby producing the modified and in illustrative embodiments genetically modified T cells and/or NK cells. It is noteworthy that although many of the aspects and embodiments provided herein are discussed in terms of RIPs, it is intended, and a skilled artisan will recognize, that many different recombinant nucleic acid/polynucleotide vectors, including but not limited to those provided herein, can be used and/or included in such methods and compositions. In illustrative embodiments wherein the recombinant nucleic acid vector is a RIP, the RIP typically comprises a fusogenic element and a binding element, which can be part of a pseudotyping element, on its surface. In some embodiments, T cells and/or NK cells are activated before being contacted by the RIP or other polynucleotide vector. In illustrative embodiments, pre-activation of the T cell and/or NK cell is not required, and an activation element, which can be any activation element provided herein, is present in a reaction mixture in which the contacting takes place. In further illustrative embodiments, the activation element is present on a surface of the replication incompetent recombinant retroviral particle. In illustrative embodiments, the activation element is anti-CD3, such as anti-CD3 scFv, or anti-CD3 scFvFc.

Many of the method aspects provided herein, include one or more of the following steps: 1) a step of collecting blood from a subject; 2) a step of contacting cells, such as NK cells and/or in illustrative embodiments T cells, which can be from the collected blood, with a recombinant vector (typically many copies thereof), in illustrative embodiments a RIP, encoding a CAR and/or a lymphoproliferative element, in a reaction mixture, where the contacting can include an optional incubation; 3) a step of washing unbound recombinant vector away from the cells in the reaction mixture; 4) a step of collecting modified cells, such as modified NK cells and/or in illustrative embodiments modified T cells in a solution, which in illustrative embodiments can be a delivery solution, to form a cell suspension, that in illustrative embodiments is a cell formulation; and 5) a step of delivering the cell formulation to a subject, in illustrative embodiments the subject from which blood was collected, for example through infusion, or in certain illustrative embodiments intradermally, intramuscularly or intratumorally, or in further illustrative embodiments, subcutaneously. It is noteworthy that in certain illustrative embodiments, the reaction mixture includes unfractionated whole blood or includes one or more cell type that is not a PBMC, and can include all or many cell types found in whole blood, including total nucleated cells (TNCs). It is noteworthy that in certain embodiments, the recombinant vector comprises a self-driving CAR, which encodes both a CAR and a lymphoproliferative element.

As a non-limiting example, in some embodiments, between 10 and 120 ml of blood is collected (or leukocytes are isolated in 10 to 120 ml by performing leukapheresis on 0.5 to 2.0 total blood volumes); the collected, unfractionated blood/isolated cells are passed through a leukoreduction filter to isolate TNCs on top of the filter; replication incompetent recombinant retroviral particles are added to the TNCs on top of the leukoreduction filter to a total reaction mixture volume of 500 µl to 10 ml to form a reaction mixture and initiate contacting; the reaction mixture is optionally incubated for any of the contacting times provided herein, as a non-limiting example, for 1-4 hours; the non-associated replication incompetent recombinant retroviral particles are washed away from cells in the reaction mixture by filtering the reaction mixture with 10 to 120 ml of wash solution; and the cells, including modified T cells and NK cells, which are retained on the TNC filter, are eluted from the filter with 2 ml to 10 ml of delivery solution, thereby forming a cell formulation suitable for introduction or reintroduction into a subject.

Some embodiments of any methods used in any aspects provided herein, which can include a step for modifying and in illustrative embodiments genetically modifying lymphocytes, PBMCs, and in illustrative embodiments NK cells and/or in further illustrative embodiments, T cells, can include a step of collecting blood from a subject. The blood includes blood components including blood cells such as lymphocytes (e.g., T cells and NK cells) that can be used in methods and compositions provided herein. In certain illustrative embodiments, the subject is a human subject afflicted with cancer (i.e., a human cancer subject). It is noteworthy that certain embodiments do not include such a step. However, in embodiments that include collecting blood from a subject, blood can be collected or obtained from a subject by any suitable method known in the art as discussed in more detail herein, and as such the collected blood or blood-derived component can be referred to as a “blood-derived product” and typically is a “peripheral blood-derived product,” since typically it is isolated from peripheral blood. For example, the blood-derived product can be collected by venipuncture or any other blood collection method known in the art, by which a sample of unfractionated whole blood is collected in a vessel, for example a blood bag, or by which leukocytes and lymphocytes are isolated from blood, such as by apheresis (e.g., leukapheresis or lymphoplasmapheresis). In some embodiments, the volume of blood (e.g., unfractionated whole blood) collected is between 1 and 5 ml, 5 and 10 ml, 10 and 15 ml, 15 and 20 ml, 20 and 25 ml, 5 and 25 ml, 25 ml and 250 ml, 25 ml and 125 ml, 50 ml and 100 ml, or 50 ml and 250 ml, 75 ml and 125 ml, 90 ml and 120 ml, or between 95 and 110 ml. In some embodiments, the volume of blood collected can be between 1, 5, 10, 15, 20, 25, 30, 35, 40, 45, 50, 55, 60, 65, 70, 75, 80, 85, 90, 95, 100, 110, 120, 130, 140, 150, 175, 200, 225, 250, 275, 300, 350, 400, 450, 500, 600, 700, 800, or 900 ml on the low end of the range and 25, 30, 35, 40, 45, 50, 55, 60, 65, 70, 75, 80, 85, 90, 95, 100, 110, 120, 130, 140, 150, 175, 200, 225, 250, 275, 300, 350, 400, 450, 500, 600, 700, 800, or 900 ml or 1 L on the high end of the range. In some embodiments, the volume of blood collected is less than 250 ml, 100 ml, 75 ml, 20 ml, 15 ml, 10 ml, or 5 ml. In some embodiments, lymphocytes (e.g., T cells and/or NK cells) can be obtained by apheresis. In some embodiments, the volume of blood taken and processed during apheresis (e.g., leukapheresis or lymphoplasmapheresis) is between 0.5, 0.6, 0.7, 0.75, 0.8, 0.9, 1, 1.25, or 1.5 total blood volumes of a subject on the low end of the range and 0.6, 0.7, 0.75, 0.8, 0.9, 1, 1.25, 1.5 1.75, 2, 2.25, or 2.5 total blood volumes of a subject on the high end of the range, for example between 0.5 and 2.5, 0.5 and 2, 0.5 and 1.5, or between 1 and 2 total blood volumes. The total blood volume of a human typically ranges from 4.5 to 6 L and thus much more blood is typically taken and processed during apheresis than if unfractionated whole blood is collected. Whether target blood cells (e.g., T cells) are obtained by apheresis or unfractionated whole blood is collected for example into a blood bag, it is contemplated that target blood cells (e.g., T cells) therein would be processed according to a method provided herein, which in certain illustrative embodiments results in the target blood cells becoming modified, genetically modified, and/or transduced. When apheresis (e.g., leukapheresis or lymphoplasmapheresis) is used to collect a cell fraction comprising T cells and/or NK cells (e.g., to provide a leukopak or a lymphoplasmapak), such cells are resuspended in solution directly or after one or more washes, to which a recombinant vector encoding a CAR is added to form a reaction mixture provided herein. Such reaction mixture can be used in any method herein. In some illustrative methods where a subject or a blood sample therefrom has a low CD3+ blood cell count, apheresis (e.g., leukapheresis or lymphoplasmapheresis) is used to collect blood cells (e.g., White blood cells or lymphocytes) for inclusion in a method provided herein.

Regardless of whether blood is collected from a subject or blood cells are obtained by apheresis, in any of the method aspects provided herein that include a step of modifying cell, for example lymphocytes (e.g. T cells and/or NK cells), a population of cells, such as lymphocytes (e.g. T cells and/or NK cells) are typically contacted with many copies of a recombinant vector, which in some embodiments are copies of a non-viral vector, and in illustrative embodiments are identical RIPs, in a reaction mixture. The contacting in any embodiment provided herein, can be performed for example in a chamber of a closed system adapted for processing of blood cells, for example within a blood bag, as discussed in more detail herein. In some embodiments, the blood bag can have 5, 10, 15, 20, 25, 50, 75, 100, 150, 200, 250, 300, 400, or 500 ml or less of blood during the contacting. In some embodiments, the blood bag can have at least 5, 10, 15, 20, 25, 50, 75, 100, 150, 200, 250, 300, 400, or 500 ml of blood during the contacting. In some embodiments, the blood bag can have between 1, 2, 3, 4, 5, 10, 15, 20, 25, and 50 ml of blood on the low end of the range and 10, 15, 20, 25, 50, 75, 100, 150, 200, 250, 300, 400, and 500 ml of blood on the high end of the range during the contacting. For example, the blood bag can have between 1 and 10 ml, 5 and 25 ml, 10 and 50 ml, 25 and 100 ml, 50 and 200 ml, or 100 and 500 ml of blood during the contacting. In some embodiments, the mixture inside the blood bag can include an anti-coagulant such as heparin. In other embodiments, the mixture inside the blood bag does not include an ant-coagulant, or does not include heparin. The transduction reaction mixture can include one or more buffers, ions, and a culture media.

With respect to retroviral particles, and in illustrative embodiments, lentiviral particles, in certain exemplary reaction mixtures provided herein, between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15, multiplicity of infection (MOI); or at least 1 and less than 6, 11, or 51 MOI; or in some embodiments, between 5 and 10 MOI units of replication incompetent recombinant retroviral particles are present. In some embodiments, the MOI can be at least 0.1, 0.5, 1, 2, 2.5, 3, 5, 10 or 15. With respect to composition and method for transducing lymphocytes in blood, in certain embodiments higher MOI can be used than in methods wherein PBMCs are isolated and used in the reaction mixtures. For example, illustrative embodiments of compositions and methods for transducing lymphocytes in whole blood, assuming 1×10⁶ PBMCs/ml of blood, can use retroviral particles with an MOI of between 1 and 50, 2 and 25, 2.5 and 20, 2.5 and 10, 4 and 6, or about 5, and in some embodiments between 5 and 20, 5 and 15, 10 and 20, or 10 and 15.

The reaction mixture of the contacting step included in some methods provided herein, or reaction mixture aspects in some embodiments, comprises at least 10% unfractionated whole blood (e.g. at least 10%, 20%, 25%, 50%, 60%, 70%, 80%, 90%, 95%, or 99% whole blood) and optionally an effective amount of an anticoagulant; or the reaction mixture further comprises at least one additional blood or blood preparation component that is not a PBMC, for example the reaction mixture comprises an effective amount of an anticoagulant and one or more blood preparation component that is not a PBMC. A percentage of whole blood is the percent by volume of a reaction mixture that was made using unfractionated whole blood. For example, where a reaction mixture is formed by adding replication incompetent recombinant retroviral particles to whole blood, and in illustrative embodiments unfractionated whole blood, the percentage of whole blood in the reaction mixture is the volume of whole blood by the total volume of the reaction mixture times 100. In illustrative embodiments such blood or blood preparation component that is not a PBMC is one or more (e.g., at least one, two, three, four, or five) or all of the following additional components:
a) erythrocytes, wherein the erythrocytes comprise between 1 and 60% of the volume of the reaction mixture;
b) neutrophils, wherein the neutrophils comprise at least 10% of the white blood cells in the reaction mixture, or wherein the reaction mixture comprises at least 10% as many neutrophils as T cells;
c) basophils, wherein the basophils comprise at least 0.05% of the white blood cells in the reaction mixture;
d) eosinophils, wherein the reaction mixture comprises at least 0.1% of the white blood cells in the reaction mixture;
e) plasma, wherein the plasma comprises at least 1% of the volume of the reaction mixture; and
f) an anticoagulant, (such blood or blood preparation components a-f above referred to herein as (“Noteworthy Non-PBMC Blood or Blood Preparation Components”)).

In any of the aspects disclosed herein that include a percentage of whole blood, the percentage is based on volume. For example, in certain embodiments at least 25% of the volume of a reaction mixture can be whole blood. Thus, in such embodiments at least 25 ml of 100 ml of such reaction mixture, would be whole blood.

The inventors have observed that surface expression of the TCR complex, including TCRα, TCRβ, and CD3, on CD4 positive (CD4+) cells and CD8 positive (CD8+) cells is reduced or “dimmed” when such cells are contacted with polynucleotide vectors (e.g., RIPs) displaying a binding polypeptide that binds the TCR complex, e.g., a T cell activation element, as is the case in certain illustrative embodiments herein. This dimming is largely the result of internalization of the TCR complex upon activation. Furthermore, the extent of this dimming increases as the concentration of a given gene vector is increased in the reaction mixture and correlates with the ability of the gene vector to activate and enter cells. Similarly, internalization of other surface polypeptides after binding to polypeptides on the surface of a gene vector results in dimming of the surface polypeptide on the cell being contacted with the gene vector and may be common during transduction using other binding polypeptides. Thus, in some embodiments, a percent reduction in surface polypeptide expression on cells contacted with a gene vector comprising a binding polypeptide compared to surface polypeptide expression on cells not contacted with the gene vector comprising a binding polypeptide is used to quantitate the potency of a gene vector and determine the appropriate dose of gene vector used to modify a population of cells. In illustrative embodiments, a percent reduction in surface TCR complex expression on cells contacted with a gene vector compared to surface TCR complex expression on cells not contacted with the gene vector is used to quantitate the potency of a gene vector and determine the appropriate dose of gene vector used to modify a population of cells. As used herein, a “Dimming Unit” (DU) is the amount of gene vector (e.g. RIR retroviral particles) that reduces the surface expression of a surface polypeptide in 1 ml of a cell mixture after contacting with the gene vector for 4 hours at 37° C. and 5% CO₂ by 50% compared to the surface expression of the surface polypeptide in the cell mixture under similar conditions but not contacted with the gene vector. The surface polypeptide is typically a binding partner of a binding polypeptide present on the surface of the gene vector. In some embodiments, the surface polypeptide is a TCR complex polypeptide. In some embodiments, the TCR complex polypeptide is CD3D, CD3E, CD3G, CD3Z, TCRα, or TCRβ. In illustrative embodiments, the binding partner is CD3 and the binding polypeptide is anti-CD3.

Because the level of expression of a binding polypeptide on the surface of a polynucleotide vector (sometimes referred to herein as a “gene vector”) will vary between different binding polypeptides and between polynucleotide vector preparations, the ability of a polynucleotide vector to reduce surface expression of a surface polypeptide should be determined for each preparation of a polynucleotide vector. In some embodiments, the ability of a polynucleotide vector to reduce surface expression of a surface polypeptide is determined based on target cell number. In some embodiments, the ability of a polynucleotide vector to reduce surface expression of a surface polypeptide is based on the volume the cells. In any of the aspects and embodiments herein, the reduction of surface expression of a surface polypeptide can be referred to as dimming the surface polypeptide. For example, if the surface expression of CD3 on a cell is reduced, then CD3 is dimmed on that cell and the cell can be called CD3-, even though the cell may still contain CD3 not expressed on its surface. Not to be limited by theory, T cells that temporarily internalize and dim CD3 are T cells and will eventually re-express CD3 on their cell surfaces such that they are again CD3+.

Accordingly, provided herein in one aspect, is a method for determining an amount of a polynucleotide vector preparation to dim surface expression of a surface polypeptide by a dimming percentage on cells in a dimming volume, comprising:
a) forming a plurality of reaction mixtures comprising a plurality of volumes of the polynucleotide vector preparation and a plurality of volumes of a cell mixture, wherein at least two of the reaction mixtures in the plurality of reaction mixtures comprise different volumes of the polynucleotide vector preparation and/or the cell mixture, wherein the cell mixture comprises a plurality of cells comprising the surface polypeptide on their surfaces, and wherein the polynucleotide vector preparation comprises a plurality of polynucleotide vectors comprising a binding polypeptide on their surfaces capable of binding the surface polypeptide;
b) incubating the reaction mixtures;
c) measuring the surface expression of the surface polypeptide in the reaction mixtures and in an uncontacted volume of the cell mixture, wherein the uncontacted volume of the cell mixture is not contacted with the polynucleotide vector preparation; and
d) determining the amount of the polynucleotide vector preparation to dim the dimming percentage of cells in the dimming volume the measured surface expression of the surface polypeptide in the reaction mixtures, the measured surface expression of the surface polypeptide in the uncontacted volume of the cell mixture, and the amounts of the polynucleotide vector preparation and the cell mixture in the reaction mixtures.

In some embodiments, the amounts of the cell mixture in the reaction mixtures is based on volume. In some embodiments, amounts of the cell mixture in the reaction mixtures is based on numbers of target cells. In some embodiments, the polynucleotide vector preparation is a viral preparation. In illustrative embodiments, the viral preparation is a replication incompetent recombinant retroviral particle preparation. In some embodiments, the dimming percentage (percentage of cells dimmed) is 50%, 60%, 70%, 75%, 80%, 85%, 90%, 95%, 96%, or 97%. In illustrative embodiments, the dimming percentage is at least or about 80%, 85%, 90%, or 95%. In some embodiments, the dimming volume is 0.25 ml, 0.5 ml, 0.75 ml, 1 ml, 2 ml, 3 ml, 4 ml, 5 ml, 10 ml, 15 ml, 20 ml, or 25 ml. In some embodiments, the surface polypeptide can be CD3D, CD3E, CD3G, CD3Z, TCRα, TCRβ, CD16A, NKp46, 2B4, CD2, DNAM, or NKG2C, NKG2D, NKG2E, NKG2F, and/or NKG2H. In some embodiments, the surface polypeptide is a TCR complex polypeptide. In some embodiments, the TCR complex polypeptide is CD3D, CD3E, CD3G, CD3Z, TCRα, or TCRβ. In illustrative embodiments, the surface polypeptide is CD3E. In some embodiments, the binding polypeptide can be any of the activation elements disclosed in the Activation Elements section herein. In such embodiments, the surface polypeptide can be the binding partner of the activation element.

In illustrative embodiments, the cell mixture is whole blood. In further illustrative embodiments, the cell mixture has been subjected to a red blood cell depletion procedure. In some embodiments, the whole blood is collected from a healthy subject, e.g., a subject that does not have or is not known or suspected to have a disease, disorder, or condition associated with associated with an elevated expression of an antigen. In some embodiments, the whole blood is collected from a subject with a disease, disorder, or condition associated with associated with an elevated expression of an antigen, wherein the polynucleotide vector will be administered to the subject or other subjects with the disease disorder, or condition. In some embodiments, the whole blood is collected from each subject and the Dimming Units are calculated for each subject individually.

In some embodiments, the reaction mixtures can be incubated for less than or about 24, 12, 10, 8, 6, 4, or 2 hours or 60, 45, 30, 15, 10, or 5 minutes, or for just an initial contacting. In some embodiments, the reaction mixtures can be incubated for between 10 minutes and 24 hours, or between 10 minutes and 8 hours, or for between 1 hour and 8 hours, or for between 1 hour and 6 hours, or in illustrative embodiments, for between 3.5 and 4.5 hours or for 4 hours. In some embodiments, the reaction mixtures can be incubated at about 10° C., 15° C., 20° C., 25° C., 30° C., 37° C., or 42° C. In some embodiments, the reaction mixtures are incubated without CO₂. In illustrative embodiments, the reaction mixtures are incubated with 5% CO₂.

In some embodiments, the surface expression of the surface polypeptide is measured by fluorescence-activated cell sorting (FACS) method. In some embodiments, the antibody used in a FACS method is GMP. In some embodiments, a CD3 antibody is used to determine surface expression of the surface polypeptide. In some embodiments, the CD3 antibody is UCHT1, OKT-3, HIT3A, TRX4, X35-3, VIT3, BMA030 (BW264/56), CLB-T3/3, CRIS7, YTH12.5, F111409, CLB-T3.4.2, TR-66, TR66.opt, HuM291, WT31, WT32, SPv-T3b, 11D8, XIII-141, XIII46, XIII-87, 12F6, T3/RW2-8C8, T3/RW24B6, OKT3D, M-T301, SMC2, F101.01, and SK7. In illustrative embodiments, the CD3 antibody is PerCP Mouse Anti-Human CD3 — Clone SK7 (BD, 347344). In some embodiments, before measuring the surface expression of the surface polypeptide, cells present in the cell mixture are separated from unbound polynucleotide vector in the incubated reaction mixture.

In an illustrative embodiment of the above method, the polynucleotide vector preparation is a replication incompetent recombinant retroviral particle preparation, the dimming percentage is 50%, the dimming volume is 1 ml, the surface polypeptide is CD3, the cell mixture is whole blood collected from a healthy subject, and the reaction mixture is incubated for 4 hours at 37° C. and 5% CO₂ and the method is used to calculate Dimming Units.

Such methods can be used to determine the amount of retroviral particles in a polynucleotide vector preparation that reduces surface polypeptide expression on cells by a specific percentage. This amount can then be used to determine an amount of the preparation of retroviral particles to use for subsequent transductions of whole blood, isolated PBMCs, or isolated TNCs. In any of the aspects and embodiments provided herein that include genetically modifying and/or transducing lymphocytes, the amount of a preparation of polynucleotide vector, for example replication incompetent recombinant retroviral particles, to add to the lymphocytes can be determined using the method above.

Dimming Units (DUs) can be used in any of the aspects or embodiments herein that include a contacting step to determine the amount of polynucleotide vector to add. As 1 DU of polynucleotide vector reduces the surface expression of the surface polypeptide by 50% in a 1 ml volume of cells, 10 DUs of polynucleotide vector reduces the surface expression of the surface polypeptide by 50% in 10 ml of a cell mixture. In some embodiments, sufficient DUs are added to a volume of cells to reduce surface expression of the surface polypeptide, for example CD3, by greater than 50%, 60%, 70%, 75%, 80%, 85%, 90%, 95%, 96%, or 97% after contacting with the polynucleotide vector compared to the surface expression of the surface polypeptide in the cell mixture under similar conditions but not contacted with the polynucleotide vector. In illustrative embodiments, sufficient DUs are added to a volume of cells to reduce surface expression of the surface polypeptide by greater than 80%, 85%, 90%, or 95% after contacting with the polynucleotide vector compared to the surface expression of the surface polypeptide in the cell mixture under similar conditions but not contacted with the polynucleotide vector. In some embodiments, at least 1, 2, 3, 4, 5, 6, 7, 8, 9, 10, 11,12, 13, 14, 15, 16, 17, 18, 19 or 20 DU are added per ml of cell mixture. In illustrative embodiments, between 5 and 20 DU, 5 and 15 DU, 10 and 20 DU, or 13 and 18 DU are added per ml of cell mixture. In some embodiments, at least 1, 2, 3, 4, 5, 6, 7, 8, 9, 10, 11,12, 13, 14, 15, 16, 17, 18, 19 or 20 DU are added per 1,000,000 target cells. In some embodiments, the target cells are lymphocytes, for example T cells or NK cells. In illustrative embodiments, the cells are in whole blood, isolated PBMCs, or isolated TNCs. In further illustrative embodiments, the cells are the remaining fraction of whole blood after lysing red blood cells. In some embodiments, sufficient DUs are added to dim a population of cells a specific percentage, for example, to dim CD3 on a population of T cells by greater than 50%, 60%, 70%, 75%, 80%, 85%, 90%, 95%, 96%, or 97%. In some embodiments, sufficient dimming units of a polynucleotide vector, and in illustrative embodiments RIP are present to increase the percentage of surface dimmed surface polypeptide, and in illustrative embodiments dimmed surface CD3-, in a population of cells, and in illustrative embodiments T cells, to at least 50%, 60%, 70%, 75%, 80%, 85%, 90%, 95%, 96%, or 97%. In any of the aspects and embodiments herein that include cells contacted with a polynucleotide vector, the composition including cells can include at least 1, 2, 3, 4, 5, 6, 7, 8, 9, 10, 11,12, 13, 14, 15, 16, 17, 18, 19 or 20 DU per ml of the cells, for example at least 1, 2, 3, 4, 5, 6, 7, 8, 9, 10, 11,12, 13, 14, 15, 16, 17, 18, 19 or 20 DU per ml of blood, cell formulation, population if cells.

In illustrative embodiments, this contacting, and the reaction mixture in which the contacting occurs, takes place within a closed cell processing system, as discussed in more detail herein. A packaging cell, and in illustrative embodiments a packaging cell line, and in particularly illustrative embodiments a packaging cell provided in certain aspects herein, can be used to produce the replication incompetent recombinant retroviral particles. The cells in the reaction mixture can be PBMCs or TNCs, and/or in reaction mixture aspects herein that provide compositions and methods for transducing lymphocytes in whole blood, an anticoagulant and/or an additional blood component, including additional types of blood cells that are not PBMCs, can be present as discussed herein. In fact, in illustrative embodiments of these composition and method aspects for transducing lymphocytes in whole blood, the reaction mixture can essentially be whole blood, and typically an anticoagulant, retroviral particles, and a relatively small amount of the solution in which the retroviral particles were delivered to the whole blood.

In reaction mixtures that relate to composition and method aspects for modifying lymphocytes in whole blood provided herein, lymphocytes, including NK cells and T cells, can be present at a lower percent of blood cells, and at a lower percentage of white blood cells, in the reaction mixture than methods that involve a PBMC enrichment procedure before forming the reaction mixture. For example, in some embodiments of these aspects, more granulocytes or neutrophils are present in the reaction mixture than NK cells or even T cells. Details regarding compositions of anticoagulants and one or more additional blood components present in the reaction mixtures of aspects for modifying lymphocytes in whole blood, are provided in detail in other sections herein. In some reaction mixture provided herein, T cells can be for example, between 10, 20, 30, or 40% of the lymphocytes of the reaction mixture on the low end of the range, and between 40, 50, 60, 70, 80, or 90% of the lymphocytes of the reaction mixture on the high end of the range. In illustrative embodiments, T cells comprise between 10 and 90%, between 20 and 90%, between 30 and 90%, between 40 and 90%, between 40 and 80%, or between 45% to 75% of the lymphocytes. In such embodiments, for example, NK cells can be present at between 1, 2, 3, 4, or 5% of the lymphocytes of the reaction mixture on the low end of the range, and between 5, 6, 7, 8, 9, 10, 11, 12, 13, or 14% of the lymphocytes of the reaction mixture on the high end of the range. In illustrative embodiments, T cells comprise between 1 and 14%, between 2 and 14%, between 3 and 14%, between 4 and 14%, between 5 and 14%, between 5 to 13%, between 5 to 12%, between 5 to 11% or between 5 to 10% of the lymphocytes of the reaction mixture. In some embodiments, T cells can be at least 1%, 2%, 3%, 4%, 5%, 6%, 7%, 8%, 9%, 10%, 15%, 20%, 30%, 40%, 50%, 60%, 70%, 80%, 90%, 95%, 96%, 97%, 98%, or 99% of the reaction mixture.As disclosed herein, composition and method aspects for transducing lymphocytes in whole blood typically do not involve any blood fractionation such as a PBMC enrichment step of a blood sample, before lymphocytes from the blood sample are contacted with recombinant nucleic acid vectors, for example retroviral particles, in the reaction mixtures disclosed herein for those aspects. Thus, in some embodiments, lymphocytes in unfractionated whole blood, are contacted with recombinant retroviral particles. However, in some embodiments, especially for some aspects in the Self-Driving CAR Methods and Compositions section herein, neutrophils/granulocytes are separated away from other blood cells before the cells are contacted with replication incompetent recombinant retroviral particles. In some embodiments, peripheral blood mononuclear cells (PBMCs) including peripheral blood lymphocytes (PBLs) such as T cell and/or NK cells, are isolated away from other components of a blood sample using for example, a PBMC enrichment procedure, before they are combined into a reaction mixture with retroviral particles. A skilled artisan will understand various methods known in the art can be used to enrich different blood fractions containing T cells and/or NK cells.

A PBMC enrichment procedure is a procedure in which PBMCs are enriched at least 25-fold, and typically at least 50-fold from other blood cell types. For example, it is believed that PBMCs make up less than 1% of blood cells in whole blood. After a PBMC enrichment procedure, at least 30%, and in some examples as many as 70% of cells isolated in the PBMC fraction are PBMCs. It is possible that even higher enrichment of PBMCs is achieved using some PBMC enrichment procedures. Various different PBMC enrichment procedures are known in the art. For example, a PBMC enrichment procedure is a ficoll density gradient centrifugation process that separates the main cell populations, such as lymphocytes, monocytes, granulocytes, and red blood cells, throughout a density gradient medium. In such a method the aqueous medium includes ficoll, a hydrophilic polysaccharide that forms the high density solution. Layering of whole blood over or under a density medium without mixing of the two layers followed by centrifugation will disperse the cells according to their densities with the PBMC fraction forming a thin white layer at the interface between the plasma and the density gradient medium (see e.g., Panda and Ravindran (2013) Isolation of Human PBMCs. BioProtoc. Vol. 3(3)). Furthermore, centripetal forces can be used to separate PBMCs from other blood components, in ficoll using the spinning force of a Sepax cell processing system.

In some embodiments, apheresis, for example leukapheresis, can be used to isolate cells, such as PBMCs. For example, AMICUS RBCX (Fresenius-Kabi) and Trima Accel (Terumo BCT) apheresis devices and kits can be used. Cells isolated by apheresis typically contain T cells, B cells, NK cells, monocytes, granulocytes, other nucleated white blood cells, red blood cells, and/or platelets. The cells collected by apheresis can be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. In some embodiments, the cells collected by apheresis can be genetically modified by any of the methods provided herein. In some embodiments, the cells collected by apheresis can be used to prepare any of the cell formulations provided herein. In some embodiments, the cells collected by apheresis can be resuspended in a variety of biocompatible buffers, such as, for example, Ca-free, Mg-free PBS. Alternatively, the undesirable components of the sample containing the cells collected by apheresis can be removed and the cells resuspended in culture media. In some embodiments, leukopheresis can be used to isolate cells, such as lymphocytes. In any of the embodiments provided herein that includes PBMCs, a leukopak can be used. In any embodiment that includes TNCs, a buffy coat can be used. In another PBMC enrichment method, an automated leukapheresis collection system (such as SPECTRA OPTIA® APHERESIS SYSTEM from Terumo BCT, Inc. Lakewood, CO 80215, USA) is used to separate the inflow of whole blood from the target PBMC fraction using high-speed centrifugation while typically returning the outflow material, such as plasma, red blood cells, and granulocytes, back to the donor, although this returning would be optional in methods provided herein. Further processing may be necessary to remove residual red blood cells and granulocytes. Both methods include a time intensive purification of the PBMCs, and the leukapheresis method requires the presence and participation of the patient during the PBMC enrichment step.

As further non-limiting examples of PBMC enrichment procedures, in some embodiments for methods of transducing, genetically modifying, and/or modifying herein, PBMCs are isolated using a Sepax or Sepax 2 cell processing system (BioSafe). In some embodiments, the PBMCs are isolated using a CliniMACS Prodigy cell processor (Miltenyi Biotec). In some embodiments, an automated apheresis separator is used which takes blood from the subject, passes the blood through an apparatus that sorts out a particular cell type (such as, for example, PBMCs), and returns the remainder back into the subject. Density gradient centrifugation can be performed after apheresis. In some embodiments, the PBMCs are isolated using a leukoreduction filter assembly. In some embodiments, magnetic bead activated cell sorting is then used for purifying a specific cell population from PBMCs, such as, for example, PBLs or a subset thereof, according to a cellular phenotype (i.e., positive selection), before they are used in a reaction mixture herein.

Other methods for purification can also be used, such as, for example, substrate adhesion, which utilizes a substrate that mimics the environment that a T cell encounters during recruitment, to purify T cells before adding them to a reaction mixture, or negative selection can be used, in which unwanted cells are targeted for removal with antibody complexes that target the unwanted cells for removal before a reaction mixture for a contacting step is formed. In some embodiments, red blood cell rosetting can be used to remove red blood cells before forming a reaction mixture. In other embodiments, hematopoietic stem cells can be removed before a contacting step, and thus in these embodiments, are not present during the contacting step. In some embodiments herein, especially for compositions and methods for transducing lymphocytes in whole blood, an ABC transporter inhibitor and/or substrate is not present before, during, or both before and during the contacting (i.e., not present in the reaction mixture in which contacting takes place) with or without optional incubating, or any step of the method.

In certain embodiments for any aspects provided herein, lymphocytes are modified and in illustrative embodiments genetically modified and/or transduced with prior activation and/or stimulation and cultured ex vivo until a desired number of cells to be delivered are achieved. In certain illustrative embodiments for any aspects provided herein, lymphocytes are modified and in illustrative embodiments genetically modified and/or transduced without prior activation or stimulation, and/or without requiring prior activation or stimulation, whether in vivo, in vitro, or ex-vivo; and/or furthermore, in some embodiments, without ex vivo or in vitro activation or stimulation after an initial contacting with or without an optional incubation, or without requiring ex vivo or in vitro activation or stimulation after an initial contacting with or without an optional incubation. In certain illustrative embodiments, the cell is activated during the contacting and is not activated at all or not activated for more than 15 minutes, 30 minutes, 1, 2, 4, or 8 hours before the contacting. In certain illustrative embodiments, activation by elements that are not present on the retroviral particle surface is not required for modifying, genetically modifying, and/or transducing the cell. Accordingly, such activation or stimulation elements are not required other than on the retroviral particle, before, during, or after the contacting. Thus, as discussed in more detail herein, these illustrative embodiments that do not require pre-activation or stimulation provide the ability to rapidly perform in vitro experiments aimed at better understanding T cells and the biologicals mechanisms, therein. Furthermore, such methods provide for much more efficient commercial production of biological products produced using PBMCs, lymphocytes, T cells, or NK cells, and development of such commercial production methods. Finally, such methods provide for more rapid ex vivo processing of lymphocytes (e.g., NK cells and especially T cells) for adoptive cell therapy, fundamentally simplifying the delivery of such therapies, for example by providing rapid point-of-care (rPOC) methods. In illustrative embodiments, some, most, at least 25%, 50%, 60%, 70%, 75%, 80%, 90%, 95%, or 99%, or all of the lymphocytes are resting when they are combined with retroviral particles to form a reaction mixture, and typically are resting when they are contacted with retroviral viral particles in a reaction mixture. In methods for modifying lymphocytes such as T cells and/or NK cells in blood or a component thereof, lymphocytes can be contacted in the typically resting state they were in when present in the collected blood in vivo immediately before collection. In some embodiments, the T cells and/or NK cells consist of between 95 and 100% resting cells (Ki-67⁻). In some embodiments, the T cell and/or NK cells that are contacted by replication incompetent recombinant retroviral particles include between 90, 91, 92, 93, 94, and 95% resting cells on the low end of the range and 96, 97, 98, 99, or 100% resting cells on the high end of the range. In some embodiments, the T cells and/or NK cells include naïve cells. In some illustrative embodiments, the subembodiments in this paragraph are included in composition and method aspects for transducing lymphocytes in whole blood.

In illustrative embodiments of aspects herein that include replication incompetent recombinant retroviral particles, contact between the T cells and/or NK cells and the replication incompetent recombinant retroviral particles can facilitate transduction of the T cells and/or NK cells by the replication incompetent recombinant retroviral particles. Not to be limited by theory, during the period of contact, the replication incompetent recombinant retroviral particles identify and bind to T cells and/or NK cells and the T cells and NK cells are “modified” as the term is used herein. At this point the retroviral and host cell membranes start to fuse, and any retroviral pseudotyping elements and/or T cell activation elements, including anti-CD3 antibodies, become integrated into the surface of the modified T cells and/or NK cells. Then, as a next step in the process of transduction, genetic material from the replication incompetent recombinant retroviral particles enters the T cells and/or NK cells at which time the T cells and/or NK cells are “genetically modified” as the phrase is used herein. It is noteworthy that such process might occur hours or even days after the contacting is initiated, and even after non-associated retroviral particles are rinsed away. Then the genetic material is typically integrated into the genomic DNA of the T cells and/or NK cells, at which time the T cells and/or NK cells are now “transduced” as the term is used herein. Similarly, cells can be modified, genetically modified, and/or transduced by recombinant vectors other than replication incompetent recombinant retroviral particles. Cells may also internalize and integrate genetic material into the genomic DNA of the T cells and/or NK cells after transfection, at which time the T cells and/or NK cells are now “stably transfected” as the term is used herein. Accordingly, in illustrative embodiments, any method for modifying and/or genetically modifying lymphocytes (e.g., T cells and/or NK cells) herein, is a method for transducing lymphocytes (e.g., T cells and/or NK cells). It is believed that by day 6 in vivo or ex vivo, after contacting is initiated, the vast majority of modified and genetically modified cells have been transduced. Methods of lentiviral transduction are known. Exemplary methods are described in, e.g., Wang et al. (2012) J. Immunother. 35(9): 689-701; Cooper et al. (2003) Blood. 101: 1637-1644; Verhoeyen et al. (2009) Methods Mol Biol. 506: 97-114; and Cavalieri et al. (2003) Blood. 102(2): 497-505. Throughout this disclosure, a transduced, or in some embodiments a stably transfected, T cell and/or NK cell includes progeny of ex vivo transduced cells that retain at least some of the nucleic acids or polynucleotides that are incorporated into the genome of a cell during the ex vivo transduction. In methods herein that recite “reintroducing” a transduced cell, it will be understood that such cell is typically not in a transduced state when it is collected from the blood of a subject.

Although in illustrative embodiments, T cells and/or NK cells are not activated prior to being contacted with a recombinant retrovirus in methods herein, a T cell activation element in illustrative embodiments is present in the reaction mixture where initial contacting of a recombinant retrovirus and lymphocytes occurs. For example, such T cell activation element can be in solution in the reaction mixture. For example, soluble anti-CD3 antibodies can be present in the reaction mixture during the contacting and optional incubation thereafter, at 25-200, 50-150, 75-125, or 100 ng/ml. In illustrative embodiments, the soluble anti-CD3 antibodies are multivalent such as bivalent, tetravalent, or a higher order valency. In illustrative embodiments, the T cell activation element is associated with the retroviral surface. The T cell activation element can be any T cell activation element provided herein. In illustrative embodiments, the T cell activation element can be anti-CD3, such as anti-CD3 scFv, or anti-CD3 scFvFc. Accordingly, in some embodiments, the replication incompetent recombinant retroviral particle can further include a T cell activation element, which in further illustrative examples is associated with the external side of the surface of the retrovirus.

The contacting step of a method for transducing and/or a method for modifying or genetically modifying lymphocytes in whole blood, provided herein, typically includes an initial step in which the retroviral particle, typically a population of retroviral particles, are brought into contact with blood cells, typically a population of blood cells that includes an anticoagulant and/or additional blood components other than PBMCs, that are not present after a PBMC enrichment procedure, while in suspension in a liquid buffer and/or media to form a transduction reaction mixture. This contacting, as in other aspects provided herein, can be followed by an optional incubating period in this reaction mixture that includes the retroviral particles and the blood cells comprising lymphocytes (e.g., T cells and/or NK cells) in suspension. In methods for modifying T cells and/or NK cells in blood or a component thereof, the reaction mixture can include at least one, two, three, four, five, or all additional blood components as disclosed herein, and in illustrative embodiments includes one or more anticoagulants.

The transduction reaction mixture in any of the aspects provided herein can be incubated at between 23 and 39° C., and in some illustrative embodiments at 37° C., in an optional incubation step after the initial contacting of retroviral particles and lymphocytes. In certain embodiments, the transduction reaction can be carried out at 37-39° C. for faster fusion/transduction. In some embodiments, the contacting step is a cold contacting step as discussed elsewhere herein, with an optional incubating step. In some embodiments, the cold contacting step is performed at temperatures less than 37° C., such as between 1° C. and 25° C. or 2° C. and 6° C. The optional incubating associated with the contacting step at these temperatures can be performed for any length of time discussed herein, for example in the Exemplary Embodiments section. In illustrative embodiments, the optional incubating associated with these temperatures is performed for 8 hours, 6 hours, 4 hours, 2 hours, and in illustrative embodiments 1 hour or less.

In some embodiments, including illustrative embodiments where contacting is performed on a filter, the contacting is carried out at a lower temperature, for example between 2° C. and 25° C., referred to herein as cold contacting, and then retroviral particles that remain unassociated in suspension are removed from the reaction mixture, for example by washing the reaction mixture over a filter, such as a leukoreduction filter, that retains leukocytes including T cells and NK cells, but not free, unassociated viral particles. The cells and retroviral particles when brought into contact in the transduction reaction mixture can be immediately processed to remove the retroviral particles that remain free in suspension and not associated with cells, from the cells. Optionally, the cells in suspension and retroviral particles whether free in suspension or associated with the cells in suspension, can be incubated for various lengths of time, as provided herein for a contacting step in a method provided herein. Before further steps, a wash can be performed, regardless of whether such cells will be studied in vitro, ex vivo or introduced into a subject. Such suspension can include allowing cells and retroviral particles to settle or causing such settling through application of a force, such as a centrifugal force, to the bottom of a vessel or chamber, as discussed in further detail herein. In illustrative embodiments, such g force is lower than the g forces used successfully in spinoculation procedures. Further contacting times and discussions regarding contacting and the optional incubation, are discussed further herein, for example in the Exemplary Embodiments section.

Current methods requiring extensive periods of ex vivo expansion of genetically modified lymphocytes before formulation and reintroduction into a subject can be utilized in some embodiments of the methods herein that include modifying such cells using a polynucleotide that include nucleic acids that encode an anti-idiotype polypeptide. There is a long-felt need for effective point-of-care adoptive cellular therapy that would allow a subject to have blood drawn (collected), lymphocytes modified and reintroduced in a single visit. In some embodiments, the methods provided herein allow for rapid ex vivo processing of lymphocytes, and in certain illustrative embodiments, PBMCs, and in other illustrative embodiments, total nucleated cells (TNCs), without an ex vivo expansion step, fundamentally simplifying the delivery of adoptive cell therapies, for example by providing such point-of-care methods, and in some illustrative embodiments, in shorter periods of time (rapid point-of-care (rPOC)). Illustrative methods are disclosed herein for modifying lymphocytes, especially NK cells and in illustrative embodiments, T cells, that are much shorter and simpler than prior methods. Accordingly, in some embodiments, the contacting step in any method provided herein of transducing, genetically modifying, and/or modifying a PBMC or a lymphocyte, typically a T cell and/or an NK cell, can be performed (or can occur) for any of the time periods provided in this specification, included, but not limited to those provided in the Exemplary Embodiments section. For example, said contacting can be for less than 24 hours, for example, less than 12 hours, less than 8 hours, less than 4 hours, less than 2 hours, less than 1 hour, less than 30 minutes or less than 15 minutes, but in each case there is at least an initial contacting step in which retroviral particles and cells come into contact in suspension in a transduction reaction mixture before retroviral particles that remain in suspension not associated with a cell, are separated from cells and typically discarded, as discussed in further detail herein. It should be noted, but not intending to be limited by theory, that it is believed that contacting begins at the time that retroviral particles and lymphocytes are combined together, typically by adding a solution containing the retroviral particles into a solution containing lymphocytes (e.g., T cells and/or NK cells).

After initial contacting, including initial cold contacting, in some embodiments there is an incubating of the reaction mixture containing cells and recombinant nucleic acid vectors, which in illustrative embodiments include nucleic acids that encode an anti-idiotype polypeptide, and in further illustrative embodiments are retroviral particles, in suspension for a specified time period without removing recombinant nucleic acid vectors (e.g., retroviral particles) that remain free in solution and not associated with cells. This incubating is sometimes referred to herein as an optional incubation. Thus, in illustrative embodiments, the contacting (including initial contacting and optional incubation) can be performed (or can occur) for between 15 minutes and 12 hours, between 15 minutes and 10 hours, or between 15 minutes and 8 hours, or any of the times included in the Exemplary Embodiments section. In certain embodiments that comprise a cold contacting step, a secondary incubation is performed by suspending cells after an optional wash step such that recombinant nucleic acid vectors, and in illustrative embodiments retroviral particles, that are not associated with a cell are washed away. In illustrative embodiments, the secondary incubation is performed at temperatures between 32° C. and 42° C., such as at 37° C. The optional secondary incubation can be performed for any length of time discussed herein. In illustrative embodiments, the optional secondary incubation is performed for 6 hours or less. Thus, in illustrative embodiments, the contacting (including initial contacting and optional incubation) can be performed (or can occur) (where as indicated in general herein the low end of a selected range is less than the high end of the selected range) for between 30 seconds or 1, 2, 5, 10, 15, 30, or 45 minutes, or 1, 2, 3, 4, 5, 6, 7, or 8 hours on the low end of the range, and between 10 minutes, 15 minutes, 30 minutes, or 1, 2, 4, 6, 8, 10, 12, 18, 24, 36, 48, and 72 hours on the high end of the range. Thus, in some embodiments, after the time when a reaction mixture is formed by adding retroviral particles to lymphocytes, the reaction mixture can be incubated for between 5 minutes on the low end of the range and 10, 15, or 30 minutes or 1, 2, 3, 4, 5, 6, 8, 10 or 12 hours on the high end of the range. In other embodiments, the reaction mixture can be incubated for between 15 minutes and 12 hours, 15 minutes and 10 hours, 15 minutes and 8 hours, 15 minutes and 6 hours, 15 minutes and 4 hours, 15 minutes and 2 hours, 15 minutes and 1 hour, 15 minutes and 45 minutes, or 15 minutes and 30 minutes. In other embodiments, the reaction mixture can be incubated for between 30 minutes and 12 hours, 30 minutes and 10 hours, 30 minutes and 8 hours, 30 minutes and 6 hours, 30 minutes and 4 hours, 30 minutes and 2 hours, 30 minutes and 1 hour, or 30 minutes and 45 minutes. In other embodiments, the reaction mixture can be incubated for between 1 hour and 12 hours, 1 hour and 8 hours, 1 hour and 4 hours, or 1 hour and 2 hours. In another illustrative embodiment, the contacting is performed for between an initial contacting step only (without any further incubating in the reaction mixture including the retroviral particles free in suspension and cells in suspension) without any further incubation in the reaction mixture, or a 5 minute, 10 minute, 15 minute, 30 minute, or 1 hour incubation in the reaction mixture.

After the indicated time period for the initial contacting and optional incubation that can be part of the contacting step, blood cells or a T cell and/or NK cell-containing fraction thereof in the reaction mixture, are separated from retroviral particles that are not associated with such cells. For example, this can be performed using a PBMC enrichment procedure (e.g., a Ficoll gradient in a Sepax unit), or in certain illustrative embodiments provided herein, by filtering the reaction mixture over a leukocyte depletion filter set assembly, and then collecting the leukocytes, which include T cells and NK cells. In another embodiment, this can be performed by centrifugation of the reaction mixture at a relative centrifugal force less than 500 g, for example 400 g, or between 300 and 490 g, or 350 and 450 g. Such centrifugation to separate retroviral particles from cells can be performed for example, for between 5 minutes and 15 minutes, or between 5 minutes and 10 minutes. In illustrative embodiments where centrifugal force is used to separate cells from retroviral particles that are not associated with cells, such g force is typically lower than the g forces used successfully in spinoculation procedures.

In some illustrative embodiments, a method provided herein in any aspect, does not involve performing a spinoculation. In such embodiments, the cell or cells are not subjected to a spinoculation of at least 400 g, 500 g, 600 g, 700 g, or 800 g for at least 15 minutes. In some embodiments, the cell or cells are not subjected to a spinoculation of at least 800 g for at least 10, 15, 20, 25, 30, 35, 40, or 45 minutes. In some embodiments, spinoculation is included as part of a contacting step. In illustrative embodiments, when spinoculation is performed there is no additional incubating as part of the contacting, as the time of the spinoculation provides the incubation time of the optional incubation discussed above. In other embodiments, there is an additional incubation after the spinoculating of between 15 minutes and 4 hours, 15 minutes and 2 hours, or 15 minutes and 1 hour. The spinoculation can be performed for example, for 30 minutes to 120 minutes, typically for at least 60 minutes, for example for 60 minutes to 180 minutes, or 60 minutes to 90 minutes. The spinoculation is typically performed in a centrifuge with a relative centrifugal force of at least 800 g, and more typically at least 1200 g, for example between 800 g and 2400 g, 800 g and 1800 g, 1200 g and 2400 g, or 1200 g and 1800 g. After the spinoculation, such methods typically involve an additional step of resuspending the pelleted cells and retroviral particles, and then removing retroviral particles that are not associated with cells according to steps discussed above when spinoculation is not performed.

The contacting step including the optional incubation therein, and the spinoculation, in embodiments that include spinoculation, can be performed at between 4° C. and 42° C. or 20° C. and 37° C. In certain illustrative embodiments, spinoculation is not performed and the contacting and associated optional incubation are carried out at 20-25° C. for 4 hours or less, 2 hours or less, 1 hour or less, 30 minutes or less, 15 minutes or less, or 15 minutes to 2 hours, 15 minutes to 1 hour, or 15 minutes to 30 minutes.

Methods of genetically modifying lymphocytes provided according to any method herein, typically include insertion into the cell of a polynucleotide comprising one or more transcriptional units encoding any transgene, for example one, two, three or all of an anti-idiotype polypeptide, a cytokine a CAR, and a lymphoproliferative element, and in illustrative embodiments includes nucleic acids encoding the anti-idiotype polypeptide according to any of the embodiments provided herein. Such anti-idiotype polypeptide, CAR and lymphoproliferative element can be provided to support the shorter and more simplified methods provided herein, which can support expansion of modified, genetically modified, and/or transduced T cells and/or NK cells after the contacting and optional incubation. Accordingly, in exemplary embodiments of any methods provided herein, lymphoproliferative elements can be delivered from the genome of the retroviral particles inside genetically modified, and/or transduced T cells and/or NK cells, such that those cells have the characteristics of increased proliferation and/or survival disclosed in the Lymphoproliferative Elements section herein. In exemplary embodiments of any methods provided herein, the genetically modified T cell or NK cell is capable of engraftment in vivo in mice and/or enrichment in vivo in mice for at least 7, 14, or 28 days. A skilled artisan will recognize that such mice may be treated or otherwise genetically modified so that any immunological differences between the genetically modified T cell and/or NK cell do not result in an immune response being elicited in the mice against any component of the lymphocyte transduced by the replication incompetent recombinant retroviral particle.

Media that can be included in a contacting step, for example when the cells and retroviral particles are initially brought into contact, or in any aspects provided herein, during optional incubation periods with the reaction mixture thereafter that include retroviral particles and cells in suspension in the media, or media that can be used during cell culturing and/or during various wash steps in any aspects provided herein, can include base media such as commercially available media for ex vivo T cell and/or NK cell culture. Non-limiting examples of such media include, X-VIVO™ 15 Chemically Defined, Serum-free Hematopoietic Cell Medium (Lonza) (2018 catalog numbers BE02-060F, BE02-00Q, BE-02-061Q, 04-744Q, or 04-418Q), ImmunoCult™-XF T Cell Expansion Medium (STEMCELL Technologies) (2018 catalog number 10981), PRIME-XV® T Cell Expansion XSFM (Irvine Scientific) (2018 catalog number 91141), AIM V® Medium CTS™ (Therapeutic Grade) (Thermo Fisher Scientific (Referred to herein as “Thermo Fisher”), or CTS™ Optimizer™ media (Thermo Fisher) (2018 catalog numbers A10221-01 (basal media (bottle)), and A10484-02 (supplement), A10221-03 (basal media (bag)), A1048501 (basal media and supplement kit (bottle)) and, A1048503 (basal media and supplement kit (bag)). Such media can be a chemically defined, serum-free formulation manufactured in compliance with cGMP, as discussed herein for kit components. The media can be xeno-free and complete. In some embodiments, the base media has been cleared by regulatory agencies for use in ex vivo cell processing, such as an FDA 510(k) cleared device. In some embodiments, the media is the basal media with or without the supplied T cell expansion supplement of 2018 catalog number A1048501 (CTS™ OpTmizer™ T Cell Expansion SFM, bottle format) or A1048503 (CTS™ OpTmizer™ T Cell Expansion SFM, bag format) both available from Thermo Fisher (Waltham, MA). Additives such as human serum albumin, human AB+ serum, and/or serum derived from the subject can be added to the transduction reaction mixture. Supportive cytokines can be added to the transduction reaction mixture, such as IL2, IL7, or IL15, or those found in human sera. dGTP can be added to the transduction reaction in certain embodiments.

In some embodiments of any method herein that includes a step of modifying lymphocytes (e.g., T cells and/or NK cells), the cells can be contacted with a retroviral particle without prior activation. The retroviral particle can include, as non-limiting examples, nucleic acids that encode an anti-idiotype polypeptide. In some embodiments of any method herein that includes a step of genetically modifying T cells and/or NK cells, the T cells and/or NK cells have not been incubated on a substrate that adheres to monocytes for more than 4 hours in one embodiment, or for more than 6 hours in another embodiment, or for more than 8 hours in another embodiment before the transduction. In one illustrative embodiment, the T cells and/or NK cells have been incubated overnight on an adherent substrate to remove monocytes before the transduction. In another embodiment, the method can include incubating the T cells and/or NK cells on an adherent substrate that binds monocytes for no more than 30 minutes, 1 hour, or 2 hours before the transduction. In another embodiment, the T cells and/or NK cells are exposed to no step of removing monocytes by an incubation on an adherent substrate before said transduction step. In another embodiment, the T cells and/or NK cells are not incubated with or exposed to a bovine serum, such as a cell culturing bovine serum, for example fetal bovine serum before or during a contacting step and/or a modifying and/or a genetically modifying and/or transduction step.

Some or all of the steps of the methods for modifying provided herein, or uses of such methods, in illustrative embodiments, are performed in a closed system. Thus, reaction mixtures formed in such methods, and modified, genetically modified, and/or transduced lymphocytes (e.g., T cells and/or NK cells) made by such methods, can be contained within such a closed system. A closed system is a cell processing system that is generally closed or fully closed to an environment, such as an environment within a room or even the environment within a hood, outside of the conduits such as tubes, and chambers, of the system in which cells are processed and/or transported. One of the greatest risks to safety and regulatory control in the cell processing procedure is the risk of contamination through frequent exposure to the environment as is found in traditional open cell culture systems. To mitigate this risk, particularly in the absence of antibiotics, some commercial processes have been developed that focus on the use of disposable (single-use) equipment. However, even with their use under aseptic conditions, there is always a risk of contamination from the opening of flasks to sample or add additional growth media. To overcome this problem, methods provided herein, which are typically ex vivo methods, are typically performed within a closed-system. Such a process is designed and can be operated such that the product is not exposed to the outside environment. Material transfer occurs via sterile connections, such as sterile tubing and sterile welded connections. Air for gas exchange can occur via a gas permeable membrane, via 0.2 µm filter to prevent environmental exposure. In some illustrative embodiments, the methods are performed on T cells, for example to provide modified and in illustrative embodiments genetically modified T cells.

Such closed system methods can be performed with commercially available devices. Different closed system devices can be used at different steps within a method and the cells can be transferred between these devices using tubing and connections such as welded, luer, spike, or clave ports to prevent exposure of the cells or media to the environment. For example, blood can be collected into an IV bag or syringe, optionally including an anticoagulant, and in some aspects, transferred to a Sepax 2 device (Biosafe) for PBMC enrichment and isolation. In other embodiments, whole blood can be filtered to collect leukocytes using a leukoreduction filter assembly. The isolated PBMCs or isolated leukocytes can be transferred to a chamber of a G-Rex device for an optional activation, a transduction and optional expansion. Alternatively, collected blood can be transduced in a blood bag, for example, the bag in which it was collected. Finally, the cells can be harvested and collected into another bag using a Sepax 2 device. The methods can be carried out in any device or combination of devices adapted for closed system T cell and/or NK cell production. Non-limiting examples of such devices include G-Rex devices (Wilson Wolf), GatheRex (Wilson Wolf), Sepax 2 (Biosafe), WAVE Bioreactors (General Electric), a CultiLife Cell Culture bag (Takara), a PermaLife bag (OriGen), CliniMACS Prodigy (Miltenyi Biotec), and VueLife bags (Saint-Gobain). In illustrative embodiments, the optional activating, the transducing and optional expanding can be performed in the same chamber or vessel in the closed system. For example, in illustrative embodiments, the chamber can be a chamber of a G-Rex device and PBMCs or leukocytes can be transferred to the chamber of the G-Rex device after they are enriched and isolated, and can remain in the same chamber of the G-Rex device until harvesting.

Methods provided herein can include transferring blood and cells therein and/or fractions thereof, as well as lymphocytes before or after they are contacted with retroviral particles, between vessels within a closed system, which thus is without environmental exposure. Vessels used in the closed system, for example, can be a tube, bag, syringe, or other container. In some embodiments, the vessel is a vessel that is used in a research facility. In some embodiments, the vessel is a vessel used in commercial production. In other embodiments, the vessel can be a collection vessel used in a blood collection process. Methods for modifying herein, typically involve a contacting step wherein lymphocytes are contacted with a replication incompetent recombinant retroviral particle. The contacting in some embodiments, can be performed in the vessel, for example, within a blood bag. Blood and various lymphocyte-containing fractions thereof, can be transferred from the vessel to another vessel (for example from a first vessel to a second vessel) within the closed system for the contacting. The second vessel can be a cell processing compartment of a closed device, such as a G-Rex device. In some embodiments, after the contacting the modified and in illustrative embodiments genetically modified (e.g., transduced) cells can be transferred to a different vessel within the closed system (i.e., without exposure to the environment). Either before or after this transfer the cells are typically washed within the closed system to remove substantially all or all of the retroviral particles. In some embodiments, a process disclosed herein, from collection of blood, to contacting (e.g., transduction), optional incubating, and post-incubation isolation and optional washing, is performed for between 15 minutes, 30 minutes, or 1, 2, 3, or 4 hours on the low end of the range, and 4, 8, 10, or 12 hours on the high end of the range.

Various embodiments of this method, as well as other aspects, such as use of NK cells and T cells made by such a method, are disclosed in detail herein. Furthermore, various elements or steps of such method aspects for transducing, genetically modifying, and/or modifying a PBMC, lymphocyte, T cell and/or NK cell, are provided herein, for example in this section and the Exemplary Embodiments section, and such methods include embodiments that are provided throughout this specification, as further discussed herein, For example, embodiments of any of the aspects for transducing, genetically modifying, and/or modifying a PBMC or a lymphocyte, for example an NK cell or in illustrative embodiments, a T cell, provided for example in this section and in the Exemplary Embodiments section, can include any of the embodiments of TIPs provided herein, including those that include one or more anti-idiotype polypeptide, lymphoproliferative element, CAR, pseudotyping element, control element, activation element, membrane-bound cytokine, miRNA, Kozak-type sequence, WPRE element, triple stop codon, and/or other element disclosed herein, and can be combined with methods herein for producing retroviral particles using a packaging cell. In certain illustrative embodiments, the retroviral particle is a lentiviral particle. Such a method for modifying, genetically modifying, and/or transducing a PBMC or a lymphocyte, such as a T cell and/or NK cell can be performed in vitro or ex vivo. A skilled artisan will recognize that details provided herein for transducing, genetically modifying, and/or modifying PBMCs or lymphocytes, such as T cell(s) and/or NK cell(s) can apply to any aspect that includes such step(s).

Introduction or reintroduction, also referred to herein as administration and readministration, of modified cells, such as modified lymphocytes and in illustrative embodiments genetically modified lymphocytes, or in some embodiments, RIPs into a subject in methods provided herein can be via any route known in the art. Such introduction or reintroduction of genetically modified lymphocytes typically involves suspending i) modified and/or ii) genetically modified and/or iiia) transduced or iiib) transfected cells, in a delivery solution to form a cell formulation that can be introduced or reintroduced into a subject as discussed in further detail herein. Such introduction of RIPs for example, can involve suspension of the RIPs in a delivery solution to form a transducing formulation that can be introduced into a subject. For example, introduction or RIPS, lymphocytes or modified lymphocytes, or reintroduction for lymphocytes or modified lymphocytes, can be delivery via infusion into a blood vessel of the subject. In some embodiments, RIPS or modified lymphocytes (e.g., T cells and/or NK cells) are administered or otherwise introduced, or reintroduced back, for lymphocytes or modified lymphocytes, into a subject by intraperitoneal administration, intratumoral administration, intramuscular administration, or in illustrative embodiments by subcutaneous administration.

Some administered cells are modified with a nucleic acid encoding a lymphoproliferative element. Not to be limited by theory, in non-limiting illustrative methods, the delivery of a polynucleotide encoding a lymphoproliferative element, to a resting T cell and/or NK cell ex vivo, which can integrate into the genome of the T cell or NK cell, provides that cell with a driver for in vivo expansion without the need for lymphodepleting the host. Thus, in illustrative embodiments, the subject is not exposed to a lymphodepleting agent within 1, 2, 3, 4, 5, 6, 7, 10, 14, 21, or 28 days, or within 1 month, 2 months, 3 months or 6 months of performing the contacting, during the contacting, and/or within 1, 2, 3, 4, 5, 6, 7, 10, 14, 21, or 28 days, or within 1 month, 2 months, 3 months or 6 months after the modified T cells and/or NK cells are reintroduced back into the subject. Furthermore, in non-limiting illustrative embodiments, methods provided herein can be performed without exposing the subject to a lymphodepleting agent during a step wherein a replication incompetent recombinant retroviral particle is in contact with resting T cells and/or resting NK cells of the subject and/or during the entire ex vivo method. Hence, methods of expanding modified and in illustrative embodiments genetically modified T cells and/or NK cells in a subject in vivo is a feature of some embodiments of the present disclosure. In certain embodiments such methods involve ex-vivo propagation of modified cells, and in illustrative embodiments, such methods are ex vivo propagation-free or substantially propagation-free.

This entire method/process from blood draw from a subject to reintroduction of modified and in illustrative embodiments genetically modified lymphocytes into the subject after ex vivo transduction of T cells and/or NK cells, in non-limiting illustrative embodiments of any aspects provided herein, can occur over a time period less than 48 hours, less than 36 hours, less than 24 hours, less than 12 hours, less than 11 hours, less than 10 hours, less than 9 hours, less than 8 hours, less than 7 hours, less than 6 hours, less than 5 hours, less than 4 hours, less than 3 hours, 2 hours, or less than 2 hours. In any of the embodiments disclosed herein, introduction or reintroduction of the modified lymphocytes can be performed by intravenous injection, intraperitoneal administration, subcutaneous administration, intratumoral, or intramuscular administration. In other embodiments, the entire method/process from blood draw/collection from a subject to reintroduction of modified lymphocytes into the subject after ex vivo transduction of T cells and/or NK cells, in non-limiting illustrative embodiments herein, occurs over a time period between 1 hour and 12 hours, 2 hours and 8 hours, 1 hour and 3 hours, 2 hours and 4 hours, 2 hours and 6 hours, 4 hours and 12 hours, 4 hours and 24 hours, 8 hours and 24 hours, 8 hours and 36 hours, 8 hours and 48 hours, 12 hours and 24 hours, 12 hours and 36 hours, or 12 hours and 48 hours, or over a time period between 15, 30, 60, 90, 120, 180, and 240 minutes on the low end of the range, and 120, 180, and 240, 300, 360, 420, and 480 minutes on the high end of the range. In other embodiments, the entire method/process from blood draw/collection from a subject to reintroduction of modified and in illustrative embodiments genetically modified lymphocytes into the subject after ex vivo transduction of T cells and/or NK cells, occurs over a time period between 1, 2, 3, 4, 6, 8, 10, and 12 hours on the low end of the range, and 8, 9, 10, 11, 12, 14, 18, 24, 36, or 48 hours on the high end of the range. In some embodiments, the modified and genetically modified T cells and/or NK cells are separated from the non-associated replication incompetent recombinant retroviral particles after the time period in which contact occurs.

Because illustrative embodiments of methods provided herein for modifying lymphocytes, and associated methods for performing adoptive cell therapy can be performed in significantly less time than prior methods, fundamental improvements in patient care and safety as well as product manufacturability are made possible. Therefore, such processes are expected to be favorable in the view of regulatory agencies responsible for approving such processes when carried out in vivo for therapeutic purposes. For example, the subject in non-limiting examples of any aspects provided herein that include a subject, can remain in the same building (e.g., infusion clinic) or room as the instrument processing their blood or sample for the entire time that the sample is being processed before modified T cells and/or NK cells are reintroduced into the patient. In non-limiting illustrative embodiments, a subject remains within line of site and/or within 100, 50, 25, or 12 feet or arm’s distance of their blood or cells that are being processed, for the entire method/process from blood draw/collection from the subject to reintroduction of blood to the subject after ex vivo transduction of T cells and/or NK cells. In other non-limiting illustrative embodiments, a subject remains awake and/or at least one person can continue to monitor the blood or cells of the subject that are being processed, throughout and/or continuously for the entire method/process from blood draw/collection from the subject to reintroduction of blood to the subject after ex vivo transduction of T cells and/or NK cells. Because of improvements provided herein, the entire method/process for adoptive cell therapy and/or for transducing resting T cells and/or NK cells from blood draw/collection from the subject to reintroduction of blood to the subject after ex vivo transduction of T cells and/or NK cells can be performed with continuous monitoring by a human. In other non-limiting illustrative embodiments, at no point the entire method/process from blood draw/collection from the subject to reintroduction of blood to the subject after ex vivo transduction of T cells and/or NK cells, are blood cells incubated in a room that does not have a person present. In other non-limiting illustrative embodiments, the entire method/process from blood draw/collection from the subject to reintroduction of blood to the subject after ex vivo transduction of T cells and/or NK cells, is performed next to the subject and/or in the same room as the subject and/or next to the bed or chair of the subject. Thus, sample identity mix-ups can be avoided, as well as long and expensive incubations over periods of days or weeks. This is further provided by the fact that methods provided herein are readily adaptable to closed and automated blood processing systems, where a blood sample and its components that will be reintroduced into the subject, only make contact with disposable, single-use components.

Methods for modifying, genetically modifying, and/or transducing cells, such as lymphocytes, and in illustrative embodiments T cells and/or NK cells provided herein, can be part of a method for delivering polynucleotides such as polynucleotide vectors to a subject, for delivering modified cells to a subject, or for performing adoptive cell therapy. In illustrative embodiments, these methods steps of collecting blood from a subject, and returning modified, genetically modified, and/or transduced lymphocytes (e.g., T cells and/or NK cells) to the subject. The present disclosure provides various treatment methods using a CAR, and in illustrative embodiments an anti-idiotype polypeptide, which can be a CAR that has an anti-idiotype extracellular recognition domain. A CAR of the present disclosure, when present in a T lymphocyte or an NK cell, can mediate cytotoxicity toward a target cell. A CAR of the present disclosure binds to an antigen present on a target cell, thereby mediating killing of a target cell by a T lymphocyte or an NK cell genetically modified to produce the CAR. The ASTR of the CAR binds to an antigen present on the surface of a target cell. The present disclosure provides methods of killing, or inhibiting the growth of, a target cell, the method involving contacting a cytotoxic immune effector cell (e.g., a cytotoxic T cell, or an NK cell) that is genetically modified to produce a subject CAR, such that the T lymphocyte or NK cell recognizes an antigen present on the surface of a target cell, and mediates killing of the target cell. The target cell can be a cancer cell, for example, and autologous cell therapy methods herein, can be methods for treating cancer, in some illustrative embodiments. In these embodiments, the subject can be a an animal or human suspected of having cancer, or more typically, a subject that is known to have cancer. In some embodiments for treating a PDL-1 positive cancer, and in illustrative embodiments a PDL-1 positive diffuse large B cell lymphoma (DLBCL), genetically modified cells can be administered in combination with an anti-PDL-1 antibody or antibody mimetic.

In some illustrative embodiments, cells are introduced or reintroduced into the subject by infusion into a vein or artery, especially when neutrophils are not present in a preparation of lymphocytes that have been contacted with retroviral particles and are ready to be reintroduced, or by subcutaneous, intratumoral, or intramuscular administration, for embodiments where at least 1%, 2%, 3%, 4%, 5%, 7.5%, 10%, 15%, 20% or 25% of the cells, or between 1% and 90%, 1% and 75%, 1% and 50%, 1% and 25%, 1 % and 20%, 1 % and 10%, 5% and 90%, 5% and 75%, 5% and 50%, 5% and 25%, 5% and 20%, 5% and 10%, 10% and 90%, 10% and 75%, 10% and 50%, 10% and 25%, or10% and 20%, of the cells in a cell formulation to be administered are neutrophils. Such embodiments, can include coadministration or sequential administration with hyaluronidase, as discussed in further detail herein. In any of the embodiments disclosed herein, the number of lymphocytes, and in illustrative embodiments modified T cells and/or NK cells, present in cell formulations provided herein and optionally reinfused or in illustrative embodiments, subcutaneously delivered into a subject can be between 1 × 10³, 2.5 × 10³, 5 × 10³, 1 × 10⁴,2.5 × 10⁴,5 × 10⁴, 1 × 10⁵, 2.5 × 10⁵, 5 × 10⁵, 1 × 10⁶, 2.5 × 10⁶, 5 × 10⁶, and 1 × 10⁷ cells/kg on the low end of the range and 5 × 10⁴, 1 × 10⁵, 2.5 × 10⁵, 5 × 10⁵, 1 × 10⁶, 2.5 × 10⁶, 5 × 10⁶, 1 × 10⁷, 2.5 × 10⁷, 5 × 10⁷, 1 × 10⁸, 1 × 10⁹, and 1 × 10¹⁰ cells/kg on the high end of the range. In certain embodiments, the number of lymphocytes, and in illustrative embodiments modified T cells and/or NK cells, present in cell formulations herein and optionally reinfused or otherwise delivered into a subject can be between 1 × 10⁴, 2.5 × 10⁴, 5 × 10⁴, and 1 × 10⁵ cells/kg on the low end of the range and 2.5 × 10⁴, 5 × 10⁴, 1 × 10⁵, 2.5 × 10⁵, 5 × 10⁵, 1 × 10⁶, 1 × 10⁷, 2.5 × 10⁷, 5 × 10⁷, and 1 × 10⁸ cells/kg on the high end of the range, or between 1 × 10⁴ cells/kg on the low end of the range and 2.5 × 10⁴, 5 × 10⁴, 1 × 10⁵, 2.5 × 10⁵, 5 × 10⁵, 1 × 10⁶, 1 × 10⁷, 2.5 × 10⁷ 5 × 10⁷, and 1 × 10⁸ cells/kg on the high end of the range. In some embodiments, the number of lymphocytes, and in illustrative embodiments T cells and/or NK cells present in cell formulations herein and optionally reinfused, or delivered intratumorally, intramuscularly, subcutaneously, or otherwise delivered into a subject can be between 5 × 10⁵, 1 × 10⁶, 2.5 × 10⁶, 5 × 10⁶, 1 × 10⁷, 2.5 × 10⁷, 5 × 10⁷, and 1 × 10⁸ cells on the low end of the range and 2.5 × 10⁶, 5 × 10⁶, 1 × 10⁷, 2.5 × 10⁷ 5 × 10⁷, 1 × 10⁸, 2.5 × 10⁸, 5 × 10⁸, and 1 × 10⁹ cells on the high end of the range. In some embodiments, the number of lymphocytes, and in illustrative embodiments T cells and/or NK cells, present in cell formulations herein and available for infusion, reinfusion, or other delivery means (e.g., subcutaneous delivery) into a 70 kg subject or patient is between 7 × 10⁵ and 2.5 × 10⁸ cells. In other embodiments, the number of lymphocytes, and in illustrative embodiments T cells and/or NK cells present in cell formulations herein and available for transduction is approximately 7 × 10⁶ plus or minus 10%.

In any of the embodiments and aspects provided herein that include a T cell, NK cell, B cell, or stem cell, the cell can be an autologous cell or an allogeneic cell. In some embodiments, the allogeneic cell can be a genetically engineered allogeneic cell. Allogeneic cells, such as allogeneic T cells, and methods for genetically engineering allogeneic cells, are known in the art. In some embodiments where the allogeneic cell is a T cell, the T cell has been genetically engineered such that at least one component of the TCR complex is functionally impaired and/or is at least partially deleted. In some embodiments, the T cell has been genetically engineered such that the expression of at least one component of the TCR complex has been reduced or eliminated. In some embodiments, the allogeneic cell can be modified such that it is missing all or part of the B2 microglobulin gene. In some embodiment, allogeneic cells can include any of the lymphoproliferative elements and/or CLEs disclosed herein. The use of lymphoproliferative elements and CLEs can reduce the required number of cells and can facilitate cell manufacturing of T cells, NK cells, B cells, or stem cells. In some embodiments, the allogeneic cell can be an immortalized cell. In any of the aspects or embodiments herein that include an allogeneic cell, steps that include collecting blood or contacting a cell with a replication incompetent recombinant retroviral particle can be eliminated. For example, for treating a subject with an allogeneic CAR-T cell, a T cell may have been previously genetically modified, and the genetically modified allogeneic CAR-T cell is administered to the subject without collecting blood from the subject. In some embodiments, the allogeneic cell is administered subcutaneously. In some embodiments, the allogeneic cell is administered intravenously. In some embodiments, the allogeneic cell is administered intraperitoneally.

In some embodiments of any of the methods provided herein for modifying lymphocytes (e.g., T cells and/or NK cells), and aspects directed to use of replication incompetent recombinant retroviral particles (RIPs) in the manufacture of a kit for modifying T cells and/or NK cells of a subject, the modified, genetically modified, and/or transduced lymphocyte (e.g., T cell and/or NK cell) or population thereof, or the RIPs in compositions provided herein without cells, such as GMP RIP compositions, are introduced or reintroduced into the subject. Introduction or reintroduction of the modified and in illustrative embodiments genetically modified lymphocytes into a subject can be via any route known in the art. For example, introduction or reintroduction can be delivery via infusion into a blood vessel of the subject. Intratumor, intraperitoneal, intramuscular, and in certain illustrative embodiments, subcutaneous. In some embodiments, the modified, genetically modified, and/or transduced lymphocyte (e.g., T cell and/or NK cell) or population thereof, undergo 4 or fewer cell divisions ex vivo prior to being introduced or reintroduced into the subject. In some embodiments, the lymphocyte(s) used in such a method are resting T cells and/or resting NK cells that are in contact with the replication incompetent recombinant retroviral particles for between 1 hour and 12 hours. In some embodiments, no more than 12 hours, 10 hours, 8 hours, 6 hours, 4 hours, 2 hours, or 1 hour pass(es) between the time blood is collected from the subject and the time the modified and/or genetically modified T cells and/or NK cells are formulated for delivery and/or are reintroduced into the subject. In some embodiments, all steps after the blood are collected and before the blood is reintroduced, are performed in a closed system in which a person monitors the closed system throughout the processing.

In some embodiments of the methods and compositions disclosed herein, the modified and in illustrative embodiments genetically modified T cells and/or NK cells are introduced back, reintroduced, reinfused or otherwise delivered into the subject without additional ex vivo manipulation, such as stimulation and/or activation of T cells and/or NKs. In the prior art methods, ex vivo manipulation is used for stimulation/activation of T cells and/or NK cells and for expansion of genetically modified T cells and/or NK cells prior to introducing the genetically modified T cells and/or NK cells into the subject. In prior art methods, this generally takes days or weeks and requires a subject to return to a clinic for a blood infusion days or weeks after an initial blood draw. In some embodiments of the methods and compositions disclosed herein, T cells and/or NK cells are not stimulated ex vivo by exposure to anti-CD3 alone or anti-CD3 in combination with co-stimulation by, for example, anti-CD28, either in solution or attached to a solid support such as, for example, beads coated with anti-CD3/anti-CD28, prior to contacting the T cells and/or NK cells with the replication incompetent recombinant retroviral particles. As such provided herein is an ex vivo propagation-free method. In other embodiments, modified and in illustrative embodiments genetically modified T cells and/or NK cells are not expanded ex vivo, or only expanded for a small number of cell divisions (e.g., 1, 2, 3, 4, or 5 rounds of cell division), but are rather expanded, or predominantly expanded, in vivo, i.e., within the subject. In some embodiments, no additional media is added to allow for further expansion of the cells. In some embodiments, no cell manufacturing of the primary blood lymphocytes (PBLs) occurs while the PBLs are contacted with the replication incompetent recombinant retroviral particles. In illustrative embodiments, no cell manufacturing of the PBLs occurs while the PBLs are ex vivo. In traditional methods of adoptive cell therapy, subjects are lymphodepleted prior to reinfusion with genetically modified T cells and or NK cells. In some embodiments, patients or subjects are not lymphodepleted prior to infusion or reinfusion with modified and/or genetically modified T cells and or NK cells. However, embodiments of the methods and compositions disclosed herein can be used on pre-activated or pre-stimulated T cells and/or NK cells as well. In some embodiments, T cells and/or NK cells can be stimulated ex vivo by exposure to anti-CD3 with or without anti-CD28 solid supports prior to contacting the T cells and/or NK cells with the replication incompetent recombinant retroviral particles. In some embodiments, the T cells and/or NK cells can be exposed to anti-CD3/anti-CD28 solid supports for less than 1, 2, 3, 4, 6, 8, 10, 12, 14, 16, 18, or 24 hours, including no exposure, before the T cells and/or NK cells are contacted the replication incompetent recombinant retroviral particles. In illustrative embodiments, the T cells and/or NK cells can be exposed to anti-CD3/anti-CD28 solid supports for less than 1, 2, 3, 4, 6, or 8 hours before the T cells and/or NK cells are contacted the replication incompetent recombinant retroviral particles.

### Enrichment of T and/or NK Cells by Positive Selection

In some embodiments, any cell in a cell mixture, cell formulation, or reaction mixture that is useful in adoptive cell therapy, referred to herein as desired cells, such as one or more cell populations of T and/or NK cells, can be enriched prior to formulation for delivery. In some embodiments, the desired cells can be enriched by positive selection prior to being contacted with a recombinant nucleic acid vector, such as a replication incompetent retroviral particle. In other embodiments, the desired cells can be enriched by positive selection after the cell mixture, cell formulation, or reaction mixture is contacted with a recombinant nucleic acid vector, such as a replication incompetent retroviral particle. In some embodiments, enriching the one or more cell populations can be performed at the same time as any of the methods of genetic modification disclosed herein, and in illustrative embodiments genetic modification with a replication incompetent retroviral particle.

Mononuclear cells (such as PBMCs) or TNCs can be isolated from a more complex cell mixture such as whole blood by density-gradient centrifugation or reverse perfusion of a leukoreduction filter assembly, respectively, as described in more detail herein. In some embodiments, the desired cells can have specific cell lineages, such as NK cells, T cells, and/or T cell subsets including naïve, effector, memory, suppressor T-cells, and/or regulatory T cells and can be enriched through the selection of cells expressing one or more surface molecules. In illustrative embodiments, the one or more surface molecules can include CD4, CD8, CD16, CD25, CD27, CD28, CD44, CD45RA, CD45RO, CD56, CD62L, CCR7, KIRs, FoxP3, and/or TCR components such as CD3. Methods using beads conjugated to antibodies directed to one or more surface molecules can be used to enrich for the desired cells using magnetic, density, and size-based separation.

### Engineered Signaling Polypeptide(s)

In some embodiments, the replication incompetent recombinant retroviral particles used to contact T cells and/or NK cells have a polynucleotide or nucleic acid having one or more transcriptional units that encode one or more engineered signaling polypeptides. In some embodiments, an engineered signaling polypeptide includes any combination of an extracellular domain (e.g., an antigen-specific targeting region or ASTR), a stalk and a transmembrane domain, combined with one or more intracellular activating domains, optionally one or more modulatory domains (such as a co-stimulatory domain), and optionally one or more T cell survival motifs. In illustrative embodiments, at least one, two, or all of the engineered signaling polypeptides is a chimeric antigen receptor (CAR) or a lymphoproliferative element (LE) such as a chimeric lymphoproliferative element (CLE). In some embodiments, at least one, two, or all of the engineered signaling polypeptides is an engineered T cell receptor (TCR). In some embodiments, when two signaling polypeptides are utilized, one encodes a lymphoproliferative element and the other encodes a chimeric antigen receptor (CAR) that includes an antigen-specific targeting region (ASTR), a transmembrane domain, and an intracellular activating domain. For any domain of an engineered signaling polypeptide disclosed herein, exemplary sequences can be found in WO2019/055946, incorporated herein in its entirety by reference. A skilled artisan will recognize that such engineered polypeptides can also be referred to as recombinant polypeptides. The engineered signaling polypeptides, such as CARs, engineered TCRs, LEs, and CLEs provided herein, are typically transgenes with respect to lymphocytes, especially T cells and NK cells, and most especially T cells and/or NK cells that are engineered using methods and compositions provided herein, to express such signaling polypeptides.

### Extracellular Domain

In some embodiments, an engineered signaling polypeptide includes an extracellular domain that is a member of a specific binding pair. For example, in some embodiments, the extracellular domain can be the extracellular domain of a cytokine receptor, or a mutant thereof, or a hormone receptor, or a mutant thereof. Such mutant extracellular domains in some embodiments have been reported to be constitutively active when expressed at least in some cell types. In illustrative embodiments, such extracellular and transmembrane domains do not include a ligand binding region. It is believed that such domains do not bind a ligand when present in an engineered signaling polypeptide and expressed in B cells, T cells, and/or NK cells. Mutations in such receptor mutants can occur in the extracellular juxtamembrane region. Not to be limited by theory, a mutation in at least some extracellular domains (and some extracellular-transmembrane domains) of engineered signaling polypeptides provided herein, are responsible for signaling of the engineered signaling polypeptide in the absence of ligand, by bringing activating chains together that are not normally together. Further embodiments regarding extracellular domains that comprise mutations in extracellular domains can be found, for example, in the Lymphoproliferative Element section herein.

In certain illustrative embodiments, the extracellular domain comprises a dimerizing motif. In an illustrative embodiment the dimerizing motif comprises a leucine zipper. In some embodiments, the leucine zipper is from a jun polypeptide, for example c-jun. Further embodiments regarding extracellular domains that comprise a dimerizing motif can be found, for example, in the Lymphoproliferative Element section herein.

In certain embodiments, the extracellular domain is an antigen-specific targeting region (ASTR), sometimes called an antigen binding domain herein. Specific binding pairs include, but are not limited to, antigen-antibody binding pairs; ligand-receptor binding pairs; and the like. Thus, a member of a specific binding pair suitable for use in an engineered signaling polypeptide of the present disclosure includes an ASTR that is an antibody, an antigen, a ligand, a receptor binding domain of a ligand, a receptor, a ligand binding domain of a receptor, and an alternative non-antibody scaffold, also referred to herein as an antibody mimetic. In any of the aspects or embodiments provided herein that include an ASTR, the ASTR can be a suitable antibody mimetic. In some embodiments, the antibody mimetic can be an affibody, an afflilin, an affimer, an affitin, an alphabody, an alphamab, an anticalin, a peptide aptamer, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a nanofitin, a leucine-rich repeat domain, a lipocalin domain, a mAb 2 or Fcab™, a nanobody, a nanoCLAMP, an OBody, a Pronectin, a single-chain TCR, a tetratricopeptide repeat domain, VHH, or a V-like domain. In any of the aspects or embodiments provided herein that include an ASTR that is an antibody, for example, an scFv, a suitable antibody mimetic can be used instead of the antibody.

An ASTR suitable for use in an engineered signaling polypeptide of the present disclosure can be any antigen-binding polypeptide. In certain embodiments, the ASTR is an antibody such as a full-length antibody, a single-chain antibody, a Fab fragment, a Fab′ fragment, a (Fab′)2 fragment, a Fv fragment, and a divalent single-chain antibody or a diabody.

In some embodiments, the ASTR is a single chain Fv (scFv). In some embodiments, the heavy chain is positioned N-terminal of the light chain in the engineered signaling polypeptide. In other embodiments, the light chain is positioned N-terminal of the heavy chain in the engineered signaling polypeptide. In any of the disclosed embodiments, the heavy and light chains can be separated by a linker as discussed in more detail herein. In any of the disclosed embodiments, the heavy or light chain can be at the N-terminus of the engineered signaling polypeptide and is typically C-terminal of another domain, such as a signal sequence or peptide.

Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use with the engineered signaling polypeptides and methods using the engineered signaling polypeptides of the present disclosure. In some instances, T cell receptor (TCR) based recognition domains.

Naturally-occurring T cell receptors include an α-subunit and a β-subunit, separately produced by unique recombination events in a T cell’s genome. Libraries of TCRs may be screened for their selectivity to a target antigen, for example, any of the antigens disclosed herein. Screens of natural and/or engineered TCRs can identify TCRs with high avidities and/or reactivities towards a target antigen. Such TCRs can be selected, cloned, and a polynucleotide encoding such a TCR can be included in a replication incompetent recombinant retroviral particle to genetically modify a lymphocyte, or in illustrative embodiments, T cell or NK cell, such that the lymphocyte expresses the engineered TCR. In some embodiments, the TCR can be a single chain TCR (scTv, single chain two-domain TCR containing VαVβ).

Certain embodiments for any aspect or embodiment herein that includes a CAR, include CARs having extracellular domains engineered to co-opt the endogenous TCR signaling complex and CD3Z signaling pathway. In one embodiment, a chimeric antigen receptor ASTR is fused to one of the endogenous TCR complex chains (e.g., TCR alpha, CD3E etc.) to promote incorporation into the TCR complex and signaling through the endogenous CD3Z chains. In other embodiments, a CAR contains a first scFv or protein that binds to the TCR complex and a second scFv or protein that binds to the target antigen (e.g., tumor antigen). In another embodiment, the TCR can be a single chain TCR (scTv, single chain two-domain TCR containing VαVβ). Finally, scFv’s may also be generated to recognize the specific MHC/peptide complex, thereby acting as a surrogate TCR. Such peptide/MHC scFv-binders may be used in many similar configurations as CARs.

In some embodiments, the ASTR can be multispecific, e.g., bispecific antibodies. Multispecific antibodies have binding specificities for at least two different sites. In certain embodiments, one of the binding specificities is for one target antigen and the other is for another target antigen. In certain embodiments, bispecific antibodies may bind to two different epitopes of a target antigen. Bispecific antibodies may also be used to localize cytotoxic agents to cells which express a target antigen. Bispecific antibodies can be prepared as full-length antibodies or antibody fragments. In some embodiments, any of the CARs of the current disclosure can be multispecific. In some embodiments, one of the ASTRs on a multispecific, e.g., bispecific, can be an anti-idiotype extracellular recognition domain as disclosed elsewhere herein.

An ASTR suitable for use in an engineered signaling polypeptide of the present disclosure, or an engineered TCR, can have a variety of antigen-binding specificities. In some cases, the antigen-binding domain is specific for an epitope present in an antigen that is expressed by (synthesized by) a target cell. In one example, the target cell is a cancer cell associated antigen. The cancer cell associated antigen can be an antigen associated with, e.g., a breast cancer cell, a B cell lymphoma cell, as a diffuse large B cell lymphoma (DLBCL) cell, a Hodgkin lymphoma cell, an ovarian cancer cell, a prostate cancer cell, a mesothelioma, a lung cancer cell (e.g., a small cell lung cancer cell), a lymphoma cell, a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a leukemia cell, a chronic myelogenous leukemia (CML) cell, a chronic lymphocytic leukemia (CLL) cell, an acute myelogenous leukemia (AML) cell, an acute lymphocytic leukemia (ALL) cell, a neuroblastoma cell, a glioma, a glioblastoma, a medulloblastoma, a colorectal cancer cell, etc. A cancer cell associated antigen may also be expressed by a non-cancerous cell. In some embodiments, the cancer cell is a PDL-1 positive cancer cell. In illustrative embodiments, the cancer cell is a PDL-1 positive DLBCL cell. In some embodiments, the cancer cell is a PDL-1 negative cell. In illustrative embodiments, the cancer cell is a PDL-1 negative DLBCL cell.

In any of the aspects or embodiments herein that include an ASTR or a recombinant TCR, the antigen can be a tumor-associated antigen or a tumor-specific antigen. In some embodiments, the tumor-associated antigen or tumor-specific antigen is Axl, ROR1, ROR2, Her2 (ERBB2), prostate stem cell antigen (PSCA), PSMA (prostate-specific membrane antigen), B cell maturation antigen (BCMA), alpha-fetoprotein (AFP), carcinoembryonic antigen (CEA), cancer antigen-125 (CA-125), CA19-9, calretinin, chromogranin, protein melan-A (melanoma antigen recognized by T lymphocytes; MART-1), myo-D1, muscle-specific actin (MSA), neurofilament, neuron-specific enolase (NSE), MUC-1, epithelial membrane protein (EMA), epithelial tumor antigen (ETA), tyrosinase, melanoma-associated antigen (MAGE), MAGE-Al, high molecular weight-melanoma associated antigen (HMW-MAA), placental alkaline phosphatase, synaptophysin, thyroglobulin, thyroid transcription factor-1, the dimeric form of the pyruvate kinase isoenzyme type M2 (tumor M2-PK), CD19, CD20, CD22, CD23, CD24, CD27, CD30, CD33, CD34, CD37, CD38, CD40, CD44, CD44v6, CD44v⅞, CD45, CD70, CD99, CD117, CD123, CD138, CD171, GD2 (ganglioside G2), EphA2, CSPG4, FAP (Fibroblast Activation Protein), kappa, lambda, 5T4, αvβ6 integrin, integrin αvβ3 (CD61), galactin, K-Ras (V-Ki-ras2 Kirsten rat sarcoma viral oncogene), Ral-B, B7-H3, B7-H6, CAIX, EGFR, EGP2, EGP40, EpCAM, fetal AchR, FRα, GD3, HLA-A1+MAGE1, HLA-A1+NY-ESO-1, HLA-DR, IL-11Ra, IL-13Ra2, Lewis-Y, Muc16, NCAM, NKG2D Ligands, PRAME, Survivin, TAG72, TEMs, VEGFR2, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, an abnormal p53 protein, New York esophageal squamous cell carcinoma antigen (NYESO1), PDL-1 and the like.

In any of the aspects or embodiments herein that include an ASTR or a recombinant TCR, the ASTR or recombinant TCR can recognize, bind to, or otherwise interact with the idiotype of a target antibody or antibody mimetic, as disclosed elsewhere herein. In such embodiments, the anti-idiotype polypeptide is a CAR or TCR wherein the ASTR is the anti-idiotype external recognition domain of an anti-idiotype polypeptide. In some embodiments, the ASTR can be any of the extracellular recognition domains of an anti-idiotype polypeptide disclosed herein. For example, the ASTR can recognize the idiotcetuximab, muromonab-CD3, efalizumab, tositumomab-i131, nebacumab, edrecolomab, catumaxomab, daclizumab, olaratumab, abciximab, rituximab, basiliximab, palivizumab, infliximab, trastuzumab, adalimumab, ibritumomab tiuxetan, omalizumab, bevacizumab, natalizumab, panitumumab, ranibizumab, eculizumab, certolizumab pegol, ustekinumab, canakinumab, golimumab, ofatumumab, tocilizumab, denosumab, belimumab, ipilimumab, brentuximab vedotin, pertuzumab, ado-trastuzumab emtansine, raxibacumab, obinutuzumab, siltuximab, ramucirumab, vedolizumab, nivolumab, pembrolizumab, blinatumomab, alemtuzumab, evolocumab, idarucizumab, necitumumab, dinutuximab, secukinumab, mepolizumab, alirocumab, daratumumab, elotuzumab, ixekizumab, reslizumab, bezlotoxumab, atezolizumab, obiltoxaximab, brodalumab, dupilumab, inotuzumab ozogamicin, guselkumab, sarilumab, avelumab, emicizumab, ocrelizumab, benralizumab, durvalumab, gemtuzumab ozogamicin, erenumab (erenumab-aooe), galcanezumab (galcanezumab-gnlm), burosumab (burosumab-twza), lanadelumab (lanadelumab-flyo), mogamulizumab (mogamulizumab-kpkc), tildrakizumab (tildrakizumab-asmn), fremanezumab (fremanezumab-vfrm), ravulizumab (ravulizumab-cwvz), cemiplimab (cemiplimab-rwlc), ibalizumab (ibalizumab-uiyk) emapalumab (emapalumab-lzsg), moxetumomab pasudotox (moxetumomab pasudotox-tdfk), caplacizumab (caplacizumab-yhdp), risankizumab (risankizumab-rzaa), polatuzumab vedotin (polatuzumab vedotin-piiq), romosozumab (romosozumab-aqqg), brolucizumab (brolucizumab-dbll), crizanlizumab (crizanlizumab-tmca), enfortumab vedotin (enfortumab vedotin-ejfv), [fam-]trastuzumab deruxtecan (fam-trastuzumab deruxtecan-nxki), teprotumumab (teprotumumab-trbw), eptinezumab (eptinezumab-jjmr), isatuximab (isatuximab-irfc), sacituzumab govitecan (sacituzumab govitecan-hziy), inebilizumab (inebilizumab-cdon), tafasitamab (tafasitamab-cxix), belantamab mafodotin (belantamab mafodotin-blmf), satralizumab (satralizumab-mwge), atoltivimab, maftivimab, odesivimab-ebgn, naxitamab-gqgk, margetuximab-cmkb, ansuvimab-zykl, evinacumab, dostarlimab (dostarlimab-gxly), loncastuximab tesirine (loncastuximab tesirine-lpyl), amivantamab (amivantamab-vmjw), aducanumab (aducanumab-avwa), tralokinumab, anifrolumab (anifrolumab-fnia), oportuzumab monatox, tisotumab vedotin, bimekizumab, narsoplimab, tezepelumab, sintilimab, inolimomb, balstilimab, ublituximab, toripalimab, omburtamab, penpulimab, tanezumab, faricimab, sutimlimab, teplizumab, and retifanlimab.

In some embodiments, a member of a specific binding pair suitable for use in an engineered signaling polypeptide is an ASTR that is a ligand for a receptor. Ligands include, but are not limited to, hormones (e.g., erythropoietin, growth hormone, leptin, etc.); cytokines (e.g., interferons, interleukins, certain hormones, etc.); growth factors (e.g., heregulin; vascular endothelial growth factor (VEGF); and the like); an integrin-binding peptide (e.g., a peptide comprising the sequence Arg-Gly-Asp (SEQ ID NO:1)); and the like.

Where the member of a specific binding pair in an engineered signaling polypeptide is a ligand, the engineered signaling polypeptide can be activated in the presence of a second member of the specific binding pair, where the second member of the specific binding pair is a receptor for the ligand. For example, where the ligand is VEGF, the second member of the specific binding pair can be a VEGF receptor, including a soluble VEGF receptor.

As noted above, in some cases, the member of a specific binding pair that is included in an engineered signaling polypeptide is an ASTR that is a receptor, e.g., a receptor for a ligand, a co-receptor, etc. The receptor can be a ligand-binding fragment of a receptor. Suitable receptors include, but are not limited to, a growth factor receptor (e.g., a VEGF receptor); a killer cell lectin-like receptor subfamily K, member 1 (NKG2D) polypeptide (receptor for MICA, MICB, and ULB6); a cytokine receptor (e.g., an IL-13 receptor; an IL-2 receptor; etc.); CD27; a natural cytotoxicity receptor (NCR) (e.g., NKP30 (NCR3/CD337) polypeptide (receptor for HLA-B-associated transcript 3 (BAT3) and B7-H6); etc.); etc.

In certain embodiments of any of the aspects provided herein that include an ASTR, the ASTR can be directed to an intermediate protein that links the ASTR with a target molecule expressed on a target cell. The intermediate protein may be endogenously expressed or introduced exogenously and may be natural, engineered, or chemically modified. In certain embodiments the ASTR can be an anti-tag ASTR such that at least one tagged intermediate, typically an antibody-tag conjugate, is included between a tag recognized by the ASTR and a target molecule, typically a protein target, expressed on a target cell. Accordingly, in such embodiments, the ASTR binds a tag and the tag is conjugated to an antibody directed against an antigen on a target cell, such as a cancer cell. Non-limiting examples of tags include fluorescein isothiocyanate (FITC), streptavidin, biotin, histidine, dinitrophenol, peridinin chlorophyll protein complex, green fluorescent protein, phycoerythrin (PE), horse radish peroxidase, palmitoylation, nitrosylation, alkaline phosphatase, glucose oxidase, and maltose binding protein. As such, the ASTR comprises a molecule that binds the tag.

### Stalk

In some embodiments, the engineered signaling polypeptide includes a stalk which is located in the portion of the engineered signaling polypeptide lying outside the cell and interposed between the ASTR and the transmembrane domain. In some embodiments, the stalk has at least 85, 90, 95, 96, 97, 98, 99, or 100% identity to a wild-type CD8 stalk region (TTTPAPRPPTPAPTIASQPLSLRPEACRPAAGG AVHTRGLDFA (SEQ ID NO:2), has at least 85, 90, 95, 96, 97, 98, 99, or 100% identity to a wild-type CD28 stalk region (FCKIEVMYPPPYLDNEKSNGTIIHVKGKHLCPSPLFPGPSKP (SEQ ID NO:3)), or has at least 85, 90, 95, 96, 97, 98, 99, or 100% identity to a wild-type immunoglobulin heavy chain stalk region. In an engineered signaling polypeptide, the stalk employed allows the antigen-specific targeting region, and typically the entire engineered signaling polypeptide, to retain increased binding to a target antigen.

The stalk region can have a length of from 4 to 250 amino acids, 10 to 250 amino acids, 4 to 200 amino acids, 4 to 100 amino acids, or 4 to 75 amino acids. In some embodiments, the stalk can have a length of from e.g., from 4 aa to 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa. In some embodiments, the stalk separates the anti-id ERD from a cell membrane to which it is attached, at a great enough distance to permit binding of the anti-id ERD to its target antibody when they come into contact. In certain embodiments, the stalk is a means for effectively separating an anti-id ERD from a cell membrane to which it is attached to permit binding of the anti-id ERD to its target antibody when they come into contact with each other.

In some embodiments, the stalk of an engineered signaling polypeptide includes at least one cysteine. For example, In some embodiments, the stalk can include the sequence Cys-Pro-Pro-Cys (SEQ ID NO:4). If present, a cysteine in the stalk of a first engineered signaling polypeptide can be available to form a disulfide bond with a stalk in a second engineered signaling polypeptide.

Stalks can include immunoglobulin hinge region amino acid sequences that are known in the art; see, e.g., Tan et al. (1990) Proc. Natl. Acad. Sci. USA 87:162; and Huck et al. (1986) Nucl. Acids Res. 14:1779. As non-limiting examples, an immunoglobulin hinge region can include a domain with at least 50, 60, 70, 75, 80, 85, 90, 95, 96, 97, 98, 99 or 100% sequence identity to a stretch of at least 10, 15, 20, or all of the amino acids of any of the following amino acid sequences: DKTHT (SEQ ID NO:5); CPPC (SEQ ID NO:4); CPEPKSCDTPPPCPR (SEQ ID NO:6) (see, e.g., Glaser et al. (2005) J. Biol. Chem. 280:41494); ELKTPLGDTTHT (SEQ ID NO:7); KSCDKTHTCP (SEQ ID NO:8); KCCVDCP (SEQ ID NO:9); KYGPPCP (SEQ ID NO: 10); EPKSCDKTHTCPPCP (SEQ ID NO:11) (human IgGl hinge); ERKCCVECPPCP (SEQ ID NO:12) (human IgG2 hinge); ELKTPLGDTTHTCPRCP (SEQ ID NO: 13) (human IgG3 hinge); SPNMVPHAHHAQ (SEQ ID NO:14) (human IgG4 hinge); and the like. The stalk can include a hinge region with an amino acid sequence of a human IgG1, IgG2, IgG3, or IgG4, hinge region. The stalk can include one or more amino acid substitutions and/or insertions and/or deletions compared to a wild-type (naturally-occurring) hinge region. For example, His229 of human IgG 1 hinge can be substituted with Tyr, so that the stalk includes the sequence EPKSCDKTYTCPPCP (SEQ ID NO:15), (see, e.g., Yan et al. (2012) J. Biol. Chem. 287:5891). The stalk can include an amino acid sequence derived from human CD8; e.g., the stalk can include the amino acid sequence: TTTPAPRPPTPAPTIASQPLSLRPEACRPAAGGAVHTRGLDFACD (SEQ ID NO:16), or a variant thereof.

### Transmembrane Domain

An engineered signaling polypeptide of the present disclosure can include transmembrane domains for insertion into a eukaryotic cell membrane. The transmembrane domain can be interposed between the ASTR and the co-stimulatory domain. The transmembrane domain can be interposed between the stalk and the co-stimulatory domain, such that the chimeric antigen receptor includes, in order from the amino terminus (N-terminus) to the carboxyl terminus (C-terminus): an ASTR; a stalk; a transmembrane domain; and an activating domain.

Any transmembrane (TM) domain that provides for insertion of a polypeptide into the cell membrane of a eukaryotic (e.g., mammalian) cell is suitable for use in aspects and embodiments disclosed herein. In some embodiments, the TM domain for any aspect provided herein that includes a CAR can include a transmembrane domain from BAFFR, C3Z, CEACAM1, CD2, CD3A, CD3B, CD3D, CD3E, CD3G, CD3Z, CD4, CD5, CD7, CD8A, CD8B, CD9, CD11A, CD11B, CD11C, CD11D, CD27, CD16, CD18, CD19, CD22, CD28, CD29, CD33, CD37, CD40, CD45, CD49A, CD49D, CD49F, CD64, CD79A, CD79B, CD80, CD84, CD86, CD96 (Tactile), CD100 (SEMA4D), CD103, C134, CD137, CD154, CD160 (BY55), CD162 (SELPLG), CD226 (DNAM1), CD229 (Ly9), CD247, CRLF2, CRTAM, CSF2RA, CSF2RB, CSF3R, EPOR, FCER1G, FCGR2C, FCGRA2, GHR, HVEM (LIGHTR), IA4, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL7RA Ins PPCL, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA1, ITGA4, ITGA6, ITGAD, ITGAE, ITGAL, ITGAM, ITGAX, ITGB1, ITGB2, ITGB7, KIRDS2, LEPR, LFA-1 (CD11a, CD18), LIFR, LTBR, MPL, NKp80 (KLRF1), OSMR, PAG/Cbp, PRLR, PSGL1, SLAM (SLAMF1, CD150, IPO-3), SLAMF4 (CD244, 2B4), SLAMF6 (NTB-A, Ly108), SLAMF7, SLAMF8 (BLAME), TNFR2, TNFRSF4, TNFRSF8, TNFRSF9, TNFRSF14, TNFRSF18, VLA1, or VLA-6, or functional mutants and/or fragments thereof.

Non-limiting examples of TM domains suitable for any of the aspects or embodiments provided herein, include a domain with at least 50, 60, 70, 75, 80, 85, 90, 95, 96, 97, 98, 99 or 100% sequence identity to a stretch of at least 10, 15, 20, or all of the amino acids of any of the following TM domains or combined stalk and TM domains: a) CD8 alpha TM (SEQ ID NO:17); b) CD8 beta TM (SEQ ID NO:18);c) CD4 stalk (SEQ ID NO:19); d) CD3Z TM (SEQ ID NO:20); e) CD28 TM (SEQ ID NO:21); f) CD134 (OX40) TM: (SEQ ID NO:22); g) CD7 TM (SEQ ID NO:23); h) CD8 stalk and TM (SEQ ID NO:24); and i) CD28 stalk and TM (SEQ ID NO:25).

As non-limiting examples, a transmembrane domain of an aspect of the invention can have at least 80%, 90%, or 95% or can have 100% sequence identity to the SEQ ID NO:17 transmembrane domain, or can have 100% sequence identity to any of the transmembrane domains from the following genes respectively: the CD8 beta transmembrane domain, the CD4 transmembrane domain, the CD3 zeta transmembrane domain, the CD28 transmembrane domain, the CD134 transmembrane domain, or the CD7 transmembrane domain.

### Intracellular Activating Domain

Intracellular activating domains suitable for use in an engineered signaling polypeptide of the present disclosure when activated, typically induce the production of one or more cytokines; increase cell death; and/or increase proliferation of CD8⁺ T cells, CD4⁺ T cells, NKT cells, γδ T cells, and/or neutrophils. Activating domains can also be referred to as activation domains herein. Activating domains can be used in CARs or in lymphoproliferative elements provided herein.

In some embodiments, the intracellular activating domain includes at least one (e.g., one, two, three, four, five, six, etc.) ITAM motifs as described below. In some embodiments, an intracellular activating domain of an aspect of the invention can have at least 80%, 90%, or 95% or can have 100% sequence identity to the CD3Z, CD3D, CD3E, CD3G, CD79A, CD79B, DAP12, FCER1G, FCGR2A, FCGR2C, DAP10/CD28, ZAP70, NKp30 (B7-H6), NKG2D, NKp44, NKp46, FcR gamma (FCER1G), FcR beta (FCER1B), FcgammaRI, FcgammaRIIA, FcgammaRIIC, FcgammaRIIIA, and FcRL5 domains as described below.

Intracellular activating domains suitable for use in an engineered signaling polypeptide of the present disclosure include immunoreceptor tyrosine-based activation motif (ITAM)-containing intracellular signaling polypeptides. An ITAM motif is YX₁X₂L/I, where X₁ and X₂ are independently any amino acid. In some embodiments, the intracellular activating domain of an engineered signaling polypeptide includes 1, 2, 3, 4, or 5 ITAM motifs. In some embodiments, an ITAM motif is repeated twice in an intracellular activating domain, where the first and second instances of the ITAM motif are separated from one another by 6 to 8 amino acids, e.g., (YX₁X₂L/I)(X₃)ₙ(YX₁X₂L/I), where n is an integer from 6 to 8, and each of the 6-8 X₃ can be any amino acid. In some embodiments, the intracellular activating domain of an engineered signaling polypeptide includes 3 ITAM motifs.

A suitable intracellular activating domain can be an ITAM motif-containing portion that is derived from a polypeptide that contains an ITAM motif. For example, a suitable intracellular activating domain can be an ITAM motif-containing domain from any ITAM motif-containing protein. Thus, a suitable intracellular activating domain need not contain the entire sequence of the entire protein from which it is derived. Examples of suitable ITAM motif-containing polypeptides include, but are not limited to: CD3Z (CD3 zeta); CD3D (CD3 delta); CD3E (CD3 epsilon); CD3G (CD3 gamma); CD79A (antigen receptor complex-associated protein alpha chain); CD79B (antigen receptor complex-associated protein beta chain) DAP12; and FCERIG (Fc epsilon receptor I gamma chain). In some embodiments, an intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all the amino acids in the following ITAM motif-containing polypeptides or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of any of the following ITAM motif-containing polypeptides: CD3 zeta chain (also known as CD3Z, T cell receptor T3 zeta chain, CD247, CD3-ZETA, CD3H, CD3Q, T3Z, TCRZ, etc.) with exemplary sequences

MKWKALFTAAILQAQLPITEAQSFGLLDPKLCYLLDGILFIYGVILTALF
LRVKFSRSADAPAYQQGQNQL[YNELNLGRREEYDVL]DKRRGRDPEMGG
KPRRKNPQEGL[YNELQKDKMAEAYSEI]GMKGERRRGKGHDGL[YQGLS
TATKDTYDAL]HMQALPPR (SEQ ID NO:26),

MKWKALFTAAILQAQLPITEAQSFGLLDPKLCYLLDGILFIYGVILTALF
LRVKFSRSADAPAYQQGQNQL[YNELNLGRREEYDVL]DKRRGRDPEMGG
KPQRRKNPQEGL[YNELQKDKMAEAYSEI]GMKGERRRGKGHDGL[YQGL
STATKDTYDAL]HMQALPPR (SEQ ID NO:27),;

RVKFSRSADAPAYQQGQNQL[YNELNLGRREEYDVL]DKRRGRDPEMGGK
PRRKNPQEGL[YNELQKDKMAEAYSEI]GMKGERRRGKGHDGL[YQGLST
ATKDTYDAL]HMQALPPR (SEQ IDNO:28),

RVKFSRSADAPAYQQGQNQL[YNELNLGRREEYDVL]DKRRGRDPEMGGK
PQRRKNPQEGL[YNELQKDKMAEAYSEI]GMKGERRRGKGHDGL[YQGLS
TATKDTYDAL]HMQALPPR (SEQ IDNO:29),

NQL[YNELNLGRREEYDVL]DKR SEQ ID NO:30); EGL[YNELQKD
KMAEAYSEI]GMK(SEQ ID NO:31),

and

DGL[YQGLSTATKDTYDAL]HMQ (SEQ ID NO:32);

T cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T cell receptor T3 delta chain; T cell surface glycoprotein CD3 delta chain; etc.) with exemplary sequences:

MEHSTFLSGLVLATLLSQVSPFKIPIEELEDRVFVNCNTSITWVEGTVGT
LLSDITRLDLGKRILDPRGIYRCNGTDIYKDKESTVQVHYRMCQSCVELD
PATVAGIIVTDVIATLLLALGVFCFAGHETGRLSGAADTQALLRNDQV[Y
QPLRDRDDAQYSHL]GGNWARNK (SEQ ID NO:33),

MEHSTFLSGLVLATLLSQVSPFKIPIEELEDRVFVNCNTSITWVEGTVGT
LLSDITRLDLGKRILDPRGIYRCNGTDIYKDKESTVQVHYRTADTQALLR
NDQV[YQPLRDRDDAQYSHL]GGNWARNK(SEQ ID NO:34),

and

DQV[YQPLRDRDDAQYSHL]GGN (SEQ ID NO:35);

T cell surface glycoprotein CD3 epsilon chain (also known as CD3e, T cell surface antigen T3/Leu-4 epsilon chain, T cell surface glycoprotein CD3 epsilon chain, AI504783, CD3, CD3epsilon, T3e, etc.) with exemplary sequences:

MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDMSVATIVIVDICITGGLLLLVYYW
SKNRKAKAKPVTRGAGAGGRQRGQNKERPPPVPNPD[YEPIRKGQRDLYS
GL]NQRRI (SEQ ID NO:36)

and

NPD[YEPIRKGQRDLYSGL]NQR (SEQ ID NO:37);

T cell surface glycoprotein CD3 gamma chain (also known as CD3G, T cell receptor T3 gamma chain, CD3-GAMMA, T3G, gamma polypeptide (TiT3 complex), etc.) with exemplary sequences:

MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQL[YQPLKDREDDQYSHL]QGNQLRRN (SEQ IDNO:38) a
nd DQL[YQPLKDREDDQYSHL]QGN (SEQ ID NO:39);

CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.) with exemplary sequences:

MPGGPGVLQALPATIFLLFLLSAVYLGPGCQALWMHKVPASLMVSLGEDA
HFQCPHNSSNNANVTWWRVLHGNYTWPPEFLGPGEDPNGTLIIQNVNKSH
GGIYVCRVQEGNESYQQSCGTYLRVRQPPPRPFLDMGEGTKNRIITAEGI
ILLFCAVVPGTLLLFRKRWQNEKLGLDAGDEYEDENL[YEGLNLDDCSMY
EDI]SRGLQGTYQDVGSLNIGDVQLEKP (SEQ ID NO:40),

MPGGPGVLQALPATIFLLFLLSAVYLGPGCQALWMHKVPASLMVSLGEDA
HFQCPHNSSNNANVTWWRVLHGNYTWPPEFLGPGEDPNEPPPRPFLDMGE
GTKNRIITAEGIILLFCAVVPGTLLLFRKRWQNEKLGLDAGDEYEDENL[
YEGLNLDDCSMYEDI]SRGLQGTYQDVGSLNIGDVQLEKP(SEQ ID NO
:41), and ENL[YEGLNLDDCSMYEDI]SRG (SEQ ID NO:42);

CD79B with exemplary sequence:

LDKDDSKAGMEEDHT[YEGLDIDQTATYEDI]VTLRTGEVKWSVGEHPGQ
E (SEQ IDNO:211);

DAP12 (also known as TYROBP; TYRO protein tyrosine kinase binding protein; KARAP;PLOSL; DNAX-activation protein 12; KAR-associated protein; TYRO protein tyrosine kinase-binding protein; killer activating receptor associated protein; killer-activating receptor-associated protein; etc.)with exemplary sequences:

MGGLEPCSRLLLLPLLLAVSGLRPVQAQAQSDCSCSTVSPGVLAGIVMGD
LVLTVLIALAVYFLGRLVPRGRGAAEAATRKQRITETESP[YQELQGQRS
DVYSDL]NTQRPYYK (SEQ ID NO:43),

MGGLEPCSRLLLLPLLLAVSGLRPVQAQAQSDCSCSTVSPGVLAGIVMGD
LVLTVLIALAVYFLGRLVPRGRGAAEATRKQRITETESP[YQELQGQRSD
VYSDL]NTQ (SEQ ID NO:44),

MGGLEPCSRLLLLPLLLAVSDCSCSTVSPGVLAGIVMGDLVLTVLIALAV
YFLGRLVPRGRGAAEAATRKQRITETESP[YQELQGQRSDVYSDL]NTQR
PYYK (SEQ ID NO:45),

MGGLEPCSRLLLLPLLLAVSDCSCSTVSPGVLAGIVMGDLVLTVLIALAV
YFLGRLVPRGRGAAEATRKQRITETESP[YQELQGQRSDVYSDL]NTQRP
YYK (SEQ ID NO:46),

and

ESP[YQELQGQRSDVYSDL]NTQ (SEQ ID NO:47);

and FCERlG (also known as FCRG; Fc epsilon receptor I gamma chain; Fc receptor gamma-chain; fc-epsilon RI-gamma; fcRgamma; fceRI gamma; high affinity immunoglobulin epsilon receptor subunit gamma; immunoglobulin E receptor, high affinity, gamma chain; etc.) with exemplary sequences:

MIPAVVLLLLLLVEQAAALGEPQLCYILDAILFLYGIVLTLLYCRLKIQV
RKAAITSYEKSDGV[YTGLSTRNQETYETL]KHEKPPQ (SEQ ID NO:
48)

and

DGV[YTGLSTRNQETYETL]KHE (SEQ IDNO:49),

where the ITAM motifs are set out in brackets.

Intracellular activating domains suitable for use in an engineered signaling polypeptide of the present disclosure include a DAP10/CD28 type signaling chain. An example of a DAP10 signaling chain is the amino acid SEQ ID NO:50. In some embodiments, a suitable intracellular activating domain includes a domain with at least 50%, 60%, 70%, 75%, 80%, 85%, 90%, 95%, 96%, 97%, 98%, 99% or 100% sequence identity to a stretch of at least 10, 15, 20, or all amino acids in SEQ ID NO:50.

An example of a CD28 signaling chain is the amino acid sequence is SEQ ID NO:51. In some embodiments, a suitable intracellular domain includes a domain with at least 50%, 60%, 70%, 75%, 80%, 85%, 90%, 95%, 96%, 97%, 98%, 99% or 100% sequence identity to a stretch of at least 10, 15, 20, or all amino acids of SEQ ID NO:51.

Intracellular activating domains suitable for use in an engineered signaling polypeptide of the present disclosure include a ZAP70 polypeptide, For example, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 300 amino acids to about 400 amino acids, from about 400 amino acids to about 500 amino acids, or from about 500 amino acids to 619 amino acids, of SEQ ID NO:52.

### Modulatory Domains

Modulatory domains can change the effect of the intracellular activating domain in the engineered signaling polypeptide, including enhancing or dampening the downstream effects of the activating domain or changing the nature of the response. Modulatory domains suitable for use in an engineered signaling polypeptide of the present disclosure include co-stimulatory domains. A modulatory domain suitable for inclusion in the engineered signaling polypeptide can have a length of from about 30 amino acids to about 70 amino acids (aa), e.g., a modulatory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, modulatory domain can have a length of from about 70 aa to about 100 aa, from about 100 aa to about 200 aa, or greater than 200 aa.

Co-stimulatory domains typically enhance and/or change the nature of the response to an activation domain. Co-stimulatory domains suitable for use in an engineered signaling polypeptide of the present disclosure are generally polypeptides derived from receptors. In some embodiments, co-stimulatory domains homodimerize. A subject co-stimulatory domain can be an intracellular portion of a transmembrane protein (i.e., the co-stimulatory domain can be derived from a transmembrane protein). In some embodiments, any of the CAR provided herein can include a costimulatory domain. In some embodiments, the co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids or an intracellular domain of 4-1BB (CD137), B7-H3, B7-HCDR3, BAFFR, BTLA, C100 (SEMA4D), CD2, CD4, CD7, CD8A, CD8B, CD11A, CD11B, CD11C, CD11D, CD18, CD19, CD27, CD28, CD28 deleted for Lck binding (ICΔ), CD29, CD30, CD40, CD49A, CD49D, CD49F, CD69, CD84, CD96 (Tactile), CD103, CD160 (BY55), CD162 (SELPLG), CD226 (DNAMl), CD229 (Ly9), a ligand that specifically binds with CD83, CDS, CEACAM1, CRLF2, CRTAM, CSF2RA, CSF2RB, CSF3R, EPOR, Fc receptor gamma chain, Fc receptor ε chain, FCGRA2, GADS, GHR, GITR, HVEM, IA4, ICAM-1, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA4, ITGA6, ITGAD, ITGAE, ITGAL, ITGAM, ITGAX, ITGB1, ITGB2, ITGB7, LAT, LEPR, LFA-1 (CD11a/CD18), LIGHT, LIFR, LMP1, LTBR, MPL, MYD88, NKG2C, NKP80 (KLRF1), OSMR, OX40, PD-1, PRLR, PSGL1, PAG/Cbp, SLAM (SLAMF1, CD150, IPO-3), SLAMF4 (C244, 2B4), SLAMF6 (NTB-A, Ly108), SLAMF7, SLAMF8 (BLAME), SLP-76, TILR2, TILR4, TILR7, TILR9, TNFR2, TNFRSF4, TNFRSF8, TNFRSF9, TNFRSF14, TNFRSF18, TRANCE/RANKL, VLA1, or VLA-6,or functional mutants and/or fragments thereof.

A co-stimulatory domain suitable for inclusion in an engineered signaling polypeptide can have a length of from about 30 amino acids to about 70 amino acids (aa), e.g., a co-stimulatory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, the co-stimulatory domain can have a length of from about 70 aa to about 100 aa, from about 100 aa to about 200 aa, or greater than 200 aa.

In some embodiments, a co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all the amino acids or from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, from about 95 aa to about 100 aa, from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, or from about 160 aa to about 185 aa (depending on how long the intracellular portion of the protein is) of an intracellular portion of: CD137 (also known as TNFRSF9; CD137; 4-1BB; CDw137; ILA; etc.) for example SEQ ID NO:53, CD28 (also known as Tp44) for example SEQ ID NO:54, CD28 deleted for Lck binding (ICΔ) for example SEQ ID NO:55, ICOS (also known as AILIM, CD278, and CVID1) for example SEQ ID NO:56, OX40 (also known as TNFRSF4, RP5-902P8.3, ACT35, CD134, OX-40, TXGP1L) for example SEQ ID NO:57, CD27 (also known as S 152, T 14, TNFRSF7, and Tp55) for example SEQ ID NO:58, BTLA (also known as BTLA1 and CD272) for example SEQ ID NO:59, CD30 (also known as TNFRSF8, D1S166E, and Ki-1), for example SEQ ID NO:60, GITR (also known as TNFRSF18, RP5-902P8.2, AITR, CD357, and GITR-D), for example SEQ ID NO:61, or HVEM (also known as TNFRSF14, RP3-395M20.6, ATAR, CD270, HVEA, HVEM, LIGHTR, and TR2), for example SEQ ID NO:62. OX40 contains a p85 PI3K binding motif at residues 34-57 and a TRAF binding motif at residues 76-102, each of SEQ ID NO: 296 (of Table 1). In some embodiments, the costimulatory domain can include the p85 PI3K binding motif of OX40. In some embodiments, the costimulatory domain can include the TRAF binding motif of OX40. Lysines corresponding to amino acids 17 and 41 of SEQ ID NO: 296 are potentially negative regulatory sites that function as parts of ubiquitin targeting motifs. In some embodiments, one or both of these Lysines in the costimulatory domain of OX40 are mutated Arginines or another amino acid.

### Linker

In some embodiments, the engineered signaling polypeptide includes a linker between any two adjacent domains. For example, a linker can be between the transmembrane domain and the first co-stimulatory domain. As another example, the ASTR can be an antibody and a linker can be between the heavy chain and the light chain. As another example, a linker can be between the ASTR and the transmembrane domain and a co-stimulatory domain. As another example, a linker can be between the co-stimulatory domain and the intracellular activating domain of the second polypeptide. As another example, the linker can be between the ASTR and the intracellular signaling domain.

The linker peptide may have any of a variety of amino acid sequences. Proteins can be joined by a spacer peptide, generally of a flexible nature, although other chemical linkages are not excluded. A linker can be a peptide of between about 1 and about 100 amino acids in length, or between about 1 and about 25 amino acids in length. These linkers can be produced by using synthetic, linker-encoding oligonucleotides to couple the proteins. Peptide linkers with a degree of flexibility can be used. The linking peptides may have virtually any amino acid sequence, bearing in mind that suitable linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, (GSGGS)ₙ, (GGS)ₙ, (GGGS)ₙ, and (GGGGS)ₙ where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited GGGGSGGGGS (SEQ ID NO:674),

GGGGSGGGGSGGGGS (SEQ ID NO:63), GGGGSGGGGSGGGGSGGG
GS (SEQ ID NO:372),

GGGGSGGGGSGGGGSGGGGSGGGGS (SEQ ID NO:675),

GGGGSGGGGSGGGGSGGGGSGGGGSGGGGS (SEQ ID NO:64), GGS
SRSS (SEQ ID NO:673),

GGGGSGGGSGGGGS (SEQ ID NO:65), GGSG (SEQ ID NO:66)
, GGSGG (SEQ ID NO:67), GSGSG(SEQ ID NO:68), GSGGG
(SEQ ID NO:69), GGGSG (SEQ ID NO:70), GSSSG (SEQ
ID NO:71), and

the like. The ordinarily skilled artisan will recognize that design of a peptide conjugated to any elements described above can include linkers that are all or partially flexible, such that the linker can include a flexible linker as well as one or more portions that confer less flexible structure.

### Combinations

In some embodiments, a polynucleotide provided by the replication incompetent recombinant retroviral particles has one or more transcriptional units that encode certain combinations of the one or more engineered signaling polypeptides. In some methods and compositions provided herein, modified and in illustrative embodiments genetically modified T cells include the combinations of the one or more engineered signaling polypeptides after transduction of T cells by the replication incompetent recombinant retroviral particles. It will be understood that the reference of a first polypeptide, a second polypeptide, a third polypeptide, etc. is for convenience and elements on a “first polypeptide” and those on a “second polypeptide” means that the elements are on different polypeptides that are referenced as first or second for reference and convention only, typically in further elements or steps to that specific polypeptide.

In some embodiments, the first engineered signaling polypeptide includes an extracellular antigen binding domain, which is capable of binding an antigen, and an intracellular signaling domain. In other embodiments, the first engineered signaling polypeptide also includes a T cell survival motif and/or a transmembrane domain. In some embodiments, the first engineered signaling polypeptide does not include a co-stimulatory domain, while in other embodiments, the first engineered signaling polypeptide does include a co-stimulatory domain.

In some embodiments, a second engineered signaling polypeptide includes a lymphoproliferative gene product and optionally an extracellular antigen binding domain. In some embodiments, the second engineered signaling polypeptide also includes one or more of the following: a T cell survival motif, an intracellular signaling domain, and one or more co-stimulatory domains. In other embodiments, when two engineered signaling polypeptides are used, at least one is a CAR.

In one embodiment, the one or more engineered signaling polypeptides are expressed under a T cell specific promoter or a general promoter under the same transcript wherein in the transcript, nucleic acids encoding the engineered signaling polypeptides are separated by nucleic acids that encode one or more internal ribosomal entry sites (IREs) or one or more protease cleavage peptides.

In certain embodiments, the polynucleotide encodes two engineered signaling polypeptides wherein the first engineered signaling polypeptide includes a first extracellular antigen binding domain, which is capable of binding to a first antigen, and a first intracellular signaling domain but not a co-stimulatory domain, and the second polypeptide includes a second extracellular antigen binding domain, which is capable of binding VEGF, and a second intracellular signaling domain, such as for example, the signaling domain of a co-stimulatory molecule. In a certain embodiment, the first antigen is PSCA, PSMA, or BCMA. In a certain embodiment, the first extracellular antigen binding domain comprises an antibody or fragment thereof (e.g., scFv), e.g., an antibody or fragment thereof specific to PSCA, PSMA, or BCMA. In a certain embodiment, the second extracellular antigen binding domain that binds VEGF is a receptor for VEGF, i.e., VEGFR. In certain embodiments, the VEGFR is VEGFR1, VEGFR2, or VEGFR3. In a certain embodiment, the VEGFR is VEGFR2.

In certain embodiments, the polynucleotide encodes two engineered signaling polypeptides wherein the first engineered signaling polypeptide includes an extracellular tumor antigen binding domain and a CD3ζ signaling domain, and the second engineered signaling polypeptide includes an antigen-binding domain, wherein the antigen is an angiogenic or vasculogenic factor, and one or more co-stimulatory molecule signaling domains. The angiogenic factor can be, e.g., VEGF. The one or more co-stimulatory molecule signaling motifs can comprise, e.g., co-stimulatory signaling domains from each of CD27, CD28, OX40, ICOS, and 4-1BB.

In certain embodiments, the polynucleotide encodes two engineered signaling polypeptides wherein the first engineered signaling polypeptide includes an extracellular tumor antigen-binding domain and a CD3ζ signaling domain, the second polypeptide comprises an antigen-binding domain, which is capable of binding to VEGF, and co-stimulatory signaling domains from each of CD27, CD28, OX40, ICOS, and 4-1BB. In a further embodiment, the first signaling polypeptide or second signaling polypeptide also has a T cell survival motif. In some embodiments, the T cell survival motif is, or is derived from, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor β (TGFβ) receptor or the TGFβ decoy receptor (TGF-β-dominant-negative receptor II (DNRII)).

In certain embodiments, the polynucleotide encodes two engineered signaling polypeptides wherein the first engineered signaling polypeptide includes an extracellular tumor antigen-binding domain and a CD3ζ signaling domain, and the second engineered signaling polypeptide includes an antigen-binding domain, which is capable of binding to VEGF, an IL-7 receptor intracellular T cell survival motif, and co-stimulatory signaling domains from each of CD27, CD28, OX40, ICOS, and 4-1BB.

In some embodiments, more than two signaling polypeptides are encoded by the polynucleotide. In certain embodiments, only one of the engineered signaling polypeptides includes an antigen binding domain that binds to a tumor-associated antigen or a tumor-specific antigen; each of the remainder of the engineered signaling polypeptides comprises an antigen binding domain that binds to an antigen that is not a tumor-associated antigen or a tumor-specific antigen. In other embodiments, two or more of the engineered signaling polypeptides include antigen binding domains that bind to one or more tumor-associated antigens or tumor-specific antigens, wherein at least one of the engineered signaling polypeptides comprises an antigen binding domain that does not bind to a tumor-associated antigen or a tumor-specific antigen.

In any of the aspects or embodiments herein that include an ASTR, the antigen can be a tumor-associated antigen or a tumor-specific antigen. In some embodiments, the tumor-associated antigen or tumor-specific antigen is Axl, ROR1, ROR2, Her2 (ERBB2), prostate stem cell antigen (PSCA), PSMA (prostate-specific membrane antigen), B cell maturation antigen (BCMA), alpha-fetoprotein (AFP), carcinoembryonic antigen (CEA), cancer antigen-125 (CA-125), CA19-9, calretinin, chromogranin, protein melan-A (melanoma antigen recognized by T lymphocytes; MART-1), myo-D1, muscle-specific actin (MSA), neurofilament, neuron-specific enolase (NSE), MUC-1, epithelial membrane protein (EMA), epithelial tumor antigen (ETA), tyrosinase, melanoma-associated antigen (MAGE), MAGE-Al, high molecular weight-melanoma associated antigen (HMW-MAA), placental alkaline phosphatase, synaptophysin, thyroglobulin, thyroid transcription factor-1, the dimeric form of the pyruvate kinase isoenzyme type M2 (tumor M2-PK), CD19, CD20, CD22, CD23, CD24, CD27, CD30, CD33, CD34, CD37, CD38, CD40, CD44, CD44v6, CD44v⅞, CD45, CD70, CD99, CD117, CD123, CD138, CD171, GD2 (ganglioside G2), EphA2, CSPG4, FAP (Fibroblast Activation Protein), kappa, lambda, 5T4, αvβ6 integrin, integrin αvβ3 (CD61), galactin, K-Ras (V-Ki-ras2 Kirsten rat sarcoma viral oncogene), Ral-B, B7-H3, B7-H6, CAIX, EGFR, EGP2, EGP40, EpCAM, fetal AchR, FRα, GD3, HLA-A1+MAGE1, HLA-A1+NY-ESO-1, HLA-DR, IL-11Ra, IL-13Rα2, Lewis-Y, Muc16, NCAM, NKG2D Ligands, PRAME, Survivin, TAG72, TEMs, VEGFR2, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, an abnormal p53 protein, New York esophageal squamous cell carcinoma antigen (NYESO1), or PDL-1.

In some embodiments, the first engineered signaling polypeptide includes a first extracellular antigen binding domain that binds a first antigen, and a first intracellular signaling domain; and a second engineered signaling polypeptide includes a second extracellular antigen binding domain that binds a second antigen, or a receptor that binds the second antigen; and a second intracellular signaling domain, wherein the second engineered signaling polypeptide does not comprise a co-stimulatory domain. In a certain embodiment, the first antigen-binding domain and the second antigen-binding domain are independently an antigen-binding portion of a receptor or an antigen-binding portion of an antibody. In a certain embodiment, either or both of the first antigen binding domain or the second antigen binding domain are scFv antibody fragments. In certain embodiments, the first engineered signaling polypeptide and/or the second engineered signaling polypeptide additionally comprises a transmembrane domain. In a certain embodiment, the first engineered signaling polypeptide or the second engineered signaling polypeptide comprises a T cell survival motif, e.g., any of the T cell survival motifs described herein.

In another embodiment, the first engineered signaling polypeptide includes a first extracellular antigen binding domain that binds HER2 and the second engineered signaling polypeptide includes a second extracellular antigen binding domain that binds MUC-1.

In another embodiment, the second extracellular antigen binding domain of the second engineered signaling polypeptide binds an interleukin.

In another embodiment, the second extracellular antigen binding domain of the second engineered signaling polypeptide binds a damage associated molecular pattern molecule (DAMP; also known as an alarmin). In other embodiments, a DAMP is a heat shock protein, chromatin-associated protein high mobility group box 1 (HMGB1), S100A8 (also known as MRP8, or calgranulin A), S100A9 (also known as MRP14, or calgranulin B), serum amyloid A (SAA), deoxyribonucleic acid, adenosine triphosphate, uric acid, or heparin sulfate.

In certain embodiments, said second antigen is an antigen on an antibody that binds to an antigen presented by a tumor cell.

In some embodiments, signal transduction activation through the second engineered signaling polypeptide is non-antigenic, but is associated with hypoxia. In certain embodiments, hypoxia is induced by activation of hypoxia-inducible factor-1α (HIF-1α), HIF-1β, HIF-2α, HIF-2β, HIF-3α, or HIF-3p.

In some embodiments, for example for modifying, genetically modifying, and/or transducing lymphocytes to be introduced or reintroduced by subcutaneous injection, expression of the one or more engineered signaling polypeptides is regulated by a control element, which is disclosed in more detail herein.

### Additional Sequences

The engineered signaling polypeptides, such as CARs, can further include one or more additional polypeptide domains, where such domains include, but are not limited to, a signal sequence; an epitope tag; an affinity domain; and a polypeptide whose presence or activity can be detected (detectable marker), for example by an antibody assay or because it is a polypeptide that produces a detectable signal. Non-limiting examples of additional domains for any of the aspects or embodiments provided herein, include a domain with at least 50%, 60%, 70%, 75%, 80%, 85%, 90%, 95%, 96%, 97%, 98%, 99% or 100% sequence identity to any of the following sequences as described below: a signal sequence, an epitope tag, an affinity domain, or a polypeptide that produces a detectable signal.

Signal sequences that are suitable for use in a subject CAR, e.g., in the first polypeptide of a subject CAR, include any eukaryotic signal sequence, including a naturally-occurring signal sequence, a synthetic (e.g., man-made) signal sequence, etc. In some embodiments, for example, the signal sequence can be the CD8 signal sequence MALPVTALLLPLALLLHAARP (SEQ ID NO:72).

Suitable epitope tags include, but are not limited to, hemagglutinin (HA; e.g., YPYDVPDYA; SEQ ID NO:73); FLAG (e.g., DYKDDDDK; SEQ ID NO:74); c-myc (e.g., EQKLISEEDL; SEQ ID NO:75), and the like.

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include His5 (HHHHH; SEQ ID NO:76), HisX6 (HHHHHH; SEQ ID NO:77), c-myc (EQKLISEEDL; SEQ ID NO:75), Flag (DYKDDDDK; SEQ ID NO:74), Strep Tag (WSHPQFEK; SEQ ID NO:78), hemagglutinin, e.g., HA Tag (YPYDVPDYA; SEQ ID NO:73), GST, thioredoxin, cellulose binding domain, RYIRS (SEQ ID NO:79), Phe-His-His-Thr (SEQ ID NO:80), chitin binding domain, S-peptide, T7 peptide, SH2 domain, C-end RNA tag, WEAAAREACCRECCARA (SEQ ID NO:81), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100proteins, parvalbumin, calbindin D9K, calbindin D28K, and calretinin, inteins, biotin, streptavidin, MyoD, Id, leucine zipper sequences, and maltose binding protein.

Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFPl, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

### Chimeric Antigen Receptor

In some aspects of the present invention, an engineered signaling polypeptide is a chimeric antigen receptor (CAR) or a polynucleotide encoding a CAR, which, for simplicity, is referred to herein as “CAR.” A CAR of the present disclosure includes: a) at least one antigen-specific targeting region (ASTR); b) a transmembrane domain; and c) an intracellular activating domain. In illustrative embodiments, the antigen-specific targeting region of the CAR is an scFv portion of an antibody to the target antigen. In illustrative embodiments, the intracellular activating domain is from CD3Z, CD3D, CD3E, CD3G, CD79A, CD79B, DAP12, FCERlG, FCGR2A, FCGR2C, DAP10/CD28, or ZAP70, and some further illustrative embodiments, from CD3z. In illustrative embodiments, the CAR further comprises a co-stimulatory domain, for example any of the co-stimulatory domains provided above in the Modulatory Domains section, and in further illustrative embodiments the co-stimulatory domain is the intracellular co-stimulatory domain of 4-1BB (CD137), CD28, ICOS, OX-40, BTLA, CD27, CD30, GITR, and HVEM. In some embodiments, the CAR includes any of the transmembrane domains listed in the Transmembrane Domain section above.

A CAR of the present disclosure can be present in the plasma membrane of a eukaryotic cell, e.g., a mammalian cell, where suitable mammalian cells include, but are not limited to, a cytotoxic cell, a T lymphocyte, a stem cell, a progeny of a stem cell, a progenitor cell, a progeny of a progenitor cell, and an NK cell, an NK-T cell, and a macrophage. When present in the plasma membrane of a eukaryotic cell, a CAR of the present disclosure is active in the presence of one or more target antigens that, in certain conditions, binds the ASTR. The target antigen is the second member of the specific binding pair. The target antigen of the specific binding pair can be a soluble (e.g., not bound to a cell) factor; a factor present on the surface of a cell such as a target cell; a factor presented on a solid surface; a factor present in a lipid bilayer; and the like. Where the ASTR is an antibody, and the second member of the specific binding pair is an antigen, the antigen can be a soluble (e.g., not bound to a cell) antigen; an antigen present on the surface of a cell such as a target cell; an antigen presented on a solid surface; an antigen present in a lipid bilayer; and the like.

In some embodiments, the ASTR of a CAR is expressed as a separate polypeptide from the intracellular signaling domain. In such embodiments, one or both of the polypeptides can include any of the transmembrane domains disclosed herein. In some embodiments, one or both of the polypeptides can include a heterologous signal sequence and/or a heterologous membrane attachment sequence. In some embodiments, the heterologous membrane attachment sequence is a GPI anchor attachment sequence.

In some instances, a CAR of the present disclosure, when present in the plasma membrane of a eukaryotic cell, and when activated by one or more target antigens, increases expression of at least one nucleic acid in the cell. For example, in some cases, a CAR of the present disclosure, when present in the plasma membrane of a eukaryotic cell, and when activated by the one or more target antigens, increases expression of at least one nucleic acid in the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the level of transcription of the nucleic acid in the absence of the one or more target antigens.

As an example, the CAR of the present disclosure can include an immunoreceptor tyrosine-based activation motif (ITAM)-containing intracellular signaling polypeptide.

A CAR of the present disclosure, when present in the plasma membrane of a eukaryotic cell, and when activated by one or more target antigens, can, in some instances, result in increased production of one or more cytokines by the cell. For example, a CAR of the present disclosure, when present in the plasma membrane of a eukaryotic cell, and when activated by the one or more target antigens, can increase production of a cytokine by the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the amount of cytokine produced by the cell in the absence of the one or more target antigens. Cytokines whose production can be increased include, but are not limited to interferon gamma (IFN-γ), tumor necrosis factor-alpha (TNF-a), IL-2, IL-15, IL-12, IL-4, IL-5, IL-10; a chemokine; a growth factor; and the like.

In some embodiments, a CAR of the present disclosure, when present in the plasma membrane of a eukaryotic cell, and when activated by one or more target antigens, can result in both an increase in transcription of a nucleic acid in the cell and an increase in production of a cytokine by the cell.

In some instances, a CAR of the present disclosure, when present in the plasma membrane of a eukaryotic cell, and when activated by one or more target antigens, results in cytotoxic activity by the cell toward a target cell that expresses on its cell surface an antigen to which the antigen-binding domain of the first polypeptide of the CAR binds. For example, where the eukaryotic cell is a cytotoxic cell (e.g., an NK cell or a cytotoxic T lymphocyte), a CAR of the present disclosure, when present in the plasma membrane of the cell, and when activated by the one or more target antigens, increases cytotoxic activity of the cell toward a target cell that expresses on its cell surface the one or more target antigens. For example, where the eukaryotic cell is an NK cell or a T lymphocyte, a CAR of the present disclosure, when present in the plasma membrane of the cell, and when activated by the one or more target antigens, increases cytotoxic activity of the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the cytotoxic activity of the cell in the absence of the one or more target antigens.

In some embodiments, a CAR of the present disclosure, when present in the plasma membrane of a eukaryotic cell, and when activated by one or more target antigens, can result in other CAR activation related events such as proliferation and expansion (either due to increased cellular division or anti-apoptotic responses).

In some embodiments, a CAR of the present disclosure, when present in the plasma membrane of a eukaryotic cell, and when activated by one or more target antigens, can result in other CAR activation related events such as intracellular signaling modulation, cellular differentiation, or cell death.

In some embodiments, CARs of the present disclosure are microenvironment restricted. This property is typically the result of the microenvironment restricted nature of the ASTR domain of the CAR. Thus, CARs of the present disclosure can have a lower binding affinity or, in illustrative embodiments, can have a higher binding affinity to one or more target antigens under a condition(s) in a microenvironment than under a condition in a normal physiological environment.

In certain illustrative embodiments, CARs provided herein comprise a co-stimulatory domain in addition to an intracellular activating domain, wherein the co-stimulatory domain is any of the intracellular signaling domains provided herein for lymphoproliferative elements (LEs), such as, for example, intracellular domains of CLEs. In certain illustrative embodiments, the co-stimulatory domains of CARs herein are first intracellular domains (P3 domains) identified herein for CLEs or P4 domains that are shown as effective intracellular signaling domains of CLEs herein in the absence of a P3 domain. Furthermore, in certain illustrative embodiments, co-stimulatory domains of CARs can comprise both a P3 and a P4 intracellular signaling domain identified herein for CLEs. Certain illustrative subembodiments include especially effective P3 and P4 partner intracellular signaling domains as identified herein for CLEs. In illustrative embodiments, the co-stimulatory domain is other than an ITAM-containing intracellular domain of a CAR either as part of the co-stimulatory domain, or in further illustrative embodiments as the only co-stimulatory domain.

In these embodiments that include a CAR with a co-stimulatory domain identified herein as an effective intracellular domain of an LE, the co-stimulatory domain of a CAR can be any intracellular signaling domain in Table 1 provided herein. Active fragments of any of the intracellular domains in Table 1 can be a co-stimulatory domain of a CAR. In illustrative embodiments, the ASTR of the CAR comprises an scFv. In illustrative embodiments, in addition to the c-stimulatory intracellular domain of a CLE, these CARs comprise an intracellular activating domain that in illustrative embodiments is a CD3Z, CD3D, CD3E, CD3G, CD79A, CD79B, DAP12, FCER1G, FCGR2A, FCGR2C. DAP10/CD28, or ZAP70 intracellular activating domain, or in further illustrative embodiments is a CD3z intracellular activating domain.

In these illustrative embodiments, the co-stimulatory domain of a CAR can comprise an intracellular domain or a functional signaling fragment thereof that includes a signaling domain from CSF2RB, CRLF2, CSF2RA, CSF3R, EPOR, GHR, IFNAR1, IFNAR2, IFNGR1, IFNGR2, IFNLR1, IL1R1, IL1RAP, IL1RL1, IL1RL2, IL2RA, IL2RB, IL2RG, IL3RA, IL5RA, IL6R, IL6ST, IL7RA, IL9R, IL10RA, IL10RB, IL11RA, IL12RB1, IL12RB2, IL13RA1, IL13RA2, IL15RA, IL17RB, IL17RC, IL17RD, IL18R1, IL18RAP, IL20RA, IL20RB, IL21R, IL22RA1, IL23R, IL27RA, IL31RA, LEPR, LIFR, LMP1, MPL, MyD88, OSMR, or PRLR. In some embodiments, the co-stimulatory domain of a CAR can include an intracellular domain or a functional signaling fragment thereof that includes a signaling domain from CSF2RB, CRLF2, CSF2RA, CSF3R, EPOR, GHR, IFNAR1, IFNAR2, IFNGR1, IFNGR2, IFNLR1, IL1R1, IL1RAP, IL1RL1, IL1RL2, IL2RA, IL2RB, IL2RG, IL3RA, IL5RA, IL6R, IL6ST, IL9R, IL10RA, IL10RB, IL11RA, IL13RA1, IL13RA2, IL17RB, IL17RC, IL17RD, IL18R1, IL18RAP, IL20RA, IL20RB, IL22RA1, IL31RA, LEPR, LIFR, LMP1, MPL, MyD88, OSMR, or PRLR. In some embodiments, the co-stimulatory domain of a CAR can include an intracellular domain or a functional fragment thereof that includes a signaling domain from CSF2RB, CSF2RA, CSF3R, EPOR, IFNGR1, IFNGR2, IL1R1, IL1RAP, IL1RL1, IL2RA, IL2RG, IL5RA, IL6R, IL9R, IL10RB, IL11RA, IL12RB1, IL12RB2, IL13RA2, IL15RA, IL17RD, IL21R, IL23R, IL27RA, IL31RA, LEPR, MPL, MyD88, or OSMR. In some embodiments, the co-stimulatory domain of a CAR can include an intracellular domain or a fragment thereof that includes a signaling domain from CSF2RB, CSF2RA, CSF3R, EPOR, IFNGR1, IFNGR2, IL1R1, IL1RAP, IL1RL1, IL2RA, IL2RG, IL5RA, IL6R, IL9R, IL10RB, IL11RA, IL13RA2, IL17RD, IL31RA, LEPR, MPL, MyD88, or OSMR. In some embodiments, the co-stimulatory domain of a CAR can include an intracellular domain or a functional signaling fragment thereof that includes a signaling domain from CSF2RB, CSF3R, IFNAR1, IFNGR1, IL2RB, IL2RG, IL6ST, IL10RA, IL12RB2, IL17RC, IL17RE, IL18R1, IL27RA, IL31RA, MPL, MyD88, OSMR, or PRLR. In some embodiments, the co-stimulatory domain of a CAR can include an intracellular domain or a functional signaling fragment thereof that includes a signaling domain from CSF2RB, CSF3R, IFNGR1, IL2RB, IL2RG, IL6ST, IL10RA, IL17RE, IL31RA, MPL, or MyD88.

In some embodiments, the co-stimulatory domain of a CAR can include an intracellular domain or a fragment thereof that includes a signaling domain from CSF3R, IL6ST, IL27RA, MPL, and MyD88. In certain illustrative subembodiments, the intracellular activating domain of the CAR is derived from CD3z.

### Recombinant T Cell Receptors (TCRs)

T Cell Receptors (TCRs) recognize specific protein fragments derived from intracellular as well as extracellular proteins. When proteins are broken into peptide fragments, they are presented on the cell surface with another protein called major histocompatibility complex, or MHC, which is called the HLA (human leukocyte antigen) complex in humans. Three different T cell antigen receptors combinations in vertebrates are αβ TCR, γδTCR and pre-TCR. Such combinations are formed by dimerization between members of dimerizing subtypes, such as an α TCR subunit and a β TCR subunit, a γ TCR subunit and a δ TCR subunit, and for pre-TCRs, a pTα subunit and a β TCR subunit. A set of TCR subunits dimerize and recognize a target peptide fragment presented in the context of an MHC. The pre-TCR is expressed only on the surface of immature αβ T cells while the αß TCR is expressed on the surface of mature αβ T cells and NK T cells, and γδTCR is expressed on the surface of γδT cells. αβTCRs on the surface of a T cell recognize the peptide presented by MHCI or MHCII and the αβ TCR on the surface of NK T cells recognize lipid antigens presented by CD1. γδTCRs can recognize MHC and MHC-like molecules, and can also recognize non-MHC molecules such as viral glycoproteins. Upon ligand recognition, αβTCRs and γδTCRs transmit activation signals through the CD3zeta chain that stimulate T cell proliferation and cytokine secretion.

TCR molecules belong to the immunoglobulin superfamily with its antigen-specific presence in the V region, where CDR3 has more variability than CDR1 and CDR2, directly determining the antigen binding specificity of the TCR. When the MHC-antigen peptide complex is recognized by a TCR, the CDR1 and CDR2 recognize and bind the sidewall of the MHC molecule antigen binding channel, and the CDR3 binds directly to the antigenic peptide. Recombinant TCRs may thus be engineered that recognize a tumor-specific protein fragment presented on MHC.

Recombinant TCR’s such as those derived from human TCRα and TCRβ pairs that recognize specific peptides with common HLAs can thus be generated with specificity to a tumor specific protein (Schmitt, TM et al., 2009). The target of recombinant TCRs may be peptides derived from any of the antigen targets for CAR ASTRs provided herein, but are more commonly derived from intracellular tumor specific proteins such as oncofetal antigens, or mutated variants of normal intracellular proteins or other cancer specific neoepitopes. In some embodiments, the recombinant TCR binds to the idiotype of an antibody. In such embodiments, the TCR is an anti-idiotype polypeptide as disclosed in more detail herein. Libraries of TCR subunits may be screened for their selectivity to a target antigen. Screens of natural and/or recombinant TCR subunits can identify sets of TCR subunits with high avidities and/or reactivities towards a target antigen. Members of such sets of TCR subunits can be selected and cloned to produce one or more polynucleotide encoding the TCR subunit.

Polynucleotides encoding such a set of TCR subunits can be included in a replication incompetent recombinant retroviral particle to genetically modify a lymphocyte, or in illustrative embodiments, a T cell or an NK cell, such that the lymphocyte expresses the recombinant TCR. Accordingly, in any aspect or embodiment provided herein that includes a polynucleotide encoding a CAR or an engineered signaling polypeptide that is a CAR, the CAR can be replaced by a set of γδTCR chains, or in illustrative embodiments αβTCR chains. TCR chains that form a set may be co-expressed using a number of different techniques to co-express the two TCR chains as is disclosed herein for expressing two or more other engineered signaling polypeptides such as CARs and lymphoproliferative elements. For example, protease cleavage epitopes such as 2A protease, internal ribosomal entry sites (IRES), and separate promoters may be used.

Several strategies have been employed to reduce the likelihood of mixed TCR dimer formation. In general, this involves modification of the constant (C) domains of the TCRα and TCRβ chains to promote the preferential pairing of the introduced TCR chains with each other, while rendering them less likely to successfully pair with endogenous TCR chains. One approach that has shown some promise in vitro involves replacement of the C domain of human TCRα and TCRβ chains with their mouse counterparts. Another approach involves mutation of the human TCRα common domain and TCRβ chain common regions to promote self-pairing, or the expression of an endogenous TCR alpha and TCR beta miRNA within the viral gene construct. Accordingly, in some embodiments provided herein that include one or more sets of TCR chains as engineered signaling polypeptides, each member of the set of TCR chains, in illustrative embodiments αβTCR chains, comprises a modified constant domain that promotes preferential pairing with each other. In some subembodiments, each member of a set of TCR chains, in illustrative embodiments αβTCR chains, comprises a mouse constant domain from the same TCR chain type, or a constant domain from the same TCR chain subtype with enough sequences derived from a mouse constant domain from the same TCR chain subtype, such that dimerization of the set of TCR chains to each other is preferred over, or occurs to the exclusion of, dimerization with human TCR chains. In other subembodiments, each member of a set of TCR chains, in illustrative embodiments αβTCR chains, comprises corresponding mutations in its constant domain, such that dimerization of the set of TCR chains to each other is preferred over, or occurs to the exclusion of, dimerization with TCR chains that have human constant domains. Such preferred or exclusive dimerization in illustrative embodiments, is under physiological conditions.

In some embodiments provided herein that include one or more sets of TCR chains as engineered signaling polypeptides, the constant regions of the members of each of the one or more sets of TCR chains are swapped. Thus, the α TCR subunit of the set has a β TCR constant region, and the β TCR subunit of the set has a α TCR constant region. Not to be limited by theory, it is believed that such swapping may prevent mispairing with endogenous counterparts.

### Lymphoproliferative Elements

Many of the embodiments provided herein include a lymphoproliferative element, or a nucleic acid encoding the same, typically as part of an engineered signaling polypeptide. Accordingly, in some aspects of the present invention, for example for modified and/or genetically modified lymphocytes to be introduced or reintroduced by subcutaneous injection, an engineered signaling polypeptide is a lymphoproliferative element (LE) such as a chimeric lymphoproliferative element (CLE). Typically, the LE comprises an extracellular domain, a transmembrane domain, and at least one intracellular signaling domain that drives proliferation, and in illustrative embodiments a second intracellular signaling domain.

The extracellular domains, transmembrane domains, and intracellular domains of LEs can vary in their respective amino acid lengths. For example, for embodiments that include a replication incompetent retroviral particle (RIP), there are limits to the length of a polynucleotide that can be packaged into a retroviral particle so LEs with shorter amino acid sequences can be advantageous in certain illustrative embodiments. In some embodiments, the overall length of the LE can be between 3 and 4000 amino acids, for example between 10 and 3000, 10 and 2000, 50 and 2000, 250 and 2000 amino acids, and, in illustrative embodiments between 50 and 1000, 100 and 1000 or 250 and 1000 amino acids. The extracellular domain, when present to form an extracellular and transmembrane domain, can be between 1 and 1000 amino acids, and is typically between 4 and 400, between 4 and 200, between 4 and 100, between 4 and 50, between 4 and 25, or between 4 and 20 amino acids. In one embodiment, the extracellular region is GGGS for an extracellular and transmembrane domain of this aspect of the invention. The transmembrane domains, or transmembrane regions of extracellular and transmembrane domains, can be between 10 and 250 amino acids, and are more typically at least 15 amino acids in length, and can be, for example, between 15 and 100, 15 and 75, 15 and 50, 15 and 40, or 15 and 30 amino acids in length. The intracellular signaling domains can be, for example, between 10 and 1000, 10 and 750, 10 and 500, 10 and 250, or 10 and 100 amino acids. In illustrative embodiments, the intracellular signaling domain can be at least 30, or between 30 and 500, 30 and 250, 30 and 150, 30 and 100, 50 and 500, 50 and 250, 50 and 150, or 50 and 100 amino acids. In some embodiments, an intracellular signaling domain for a particular gene is at least 50%, 60%, 70%, 75%, 80%, 85%, 90%, 95%, 98%, 99% or 100% identical to at least 10, 25, 30, 40, 50, or all the amino acids from a sequence of that intracellular signaling domain, such as a sequence provided herein for that intracellular domain, up to the size of the entire intracellular domain sequence, and can include for example, up to an additional 1, 2, 3, 4, 5, 10, 20, or 25 amino acids, provided that such sequence still is capable of providing any of the properties of LEs disclosed herein.

In some embodiments, the lymphoproliferative element can include a first and/or second intracellular signaling domain. In some embodiments, the first and/or second intracellular signaling domain can include CD2, CD3D, CD3E, CD3G, CD4, CD8A, CD8B, CD27, mutated Delta Lck CD28, CD28, CD40, CD79A, CD79B, CRLF2, CSF2RB, CSF2RA, CSF3R, EPOR, FCER1G, FCGR2C, FCGRA2, GHR, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, LEPR, LIFR, LMP1, MPL, MYD88, OSMR, PRLR, TNFRSF4, TNFRSF8, TNFRSF9, TNFRSF14, or TNFRSF18, or functional mutants and/or fragments thereof. In illustrative embodiments, the first intracellular signaling domain can include MyD88, or a functional mutant and/or fragment thereof. In further illustrative embodiments, the first intracellular signaling domain can include MyD88, or a functional mutant and/or fragment thereof, and the second intracellular signaling domain can include ICOS, TNFRSF4, or TNSFR18, or functional mutants and/or fragments thereof. In some embodiments, the first intracellular domain is MyD88 and the second intracellular domain is an ITAM-containing intracellular domain, for example, an intracellular domain from CD3Z, CD3D, CD3E, CD3G, CD79A, CD79B, DAP12, FCER1G, FCGR2A, FCGR2C, DAP10/CD28, or ZAP70. In some embodiments, the second intracellular signaling domain can include TNFRSF18, or a functional mutant and/or fragment thereof.

In some embodiments, the lymphoproliferative element can include a fusion of an extracellular domain and a transmembrane domain. In some embodiments, the fusion of an extracellular domain and a transmembrane domain can include eTAG IL7RA Ins PPCL (interleukin 7 receptor), Myc LMP1, LMP1, eTAG CRLF2, eTAG CSF2RB, eTAG CSF3R, eTAG EPOR, eTAG GHR, eTAG truncated after Fn F523C IL27RA, or eTAG truncated after Fn S505N MPL, or functional mutants and/or fragments thereof. In some embodiments, the lymphoproliferative element can include an extracellular domain. In some embodiments, the extracellular domain can include cell tag with 0, 1, 2, 3, or 4 additional alanines at the carboxy terminus. In some embodiments, the extracellular domain can include Myc or an eTAG with 0, 1, 2, 3, or 4 additional alanines at the carboxy terminus, or functional mutants and/or fragments thereof. For any embodiment of a lymphoproliferative element disclosed herein that includes a cell tag, there is a corresponding embodiment that is identical but lacks the cell tag and optionally lacks any linker sequence that connected the cell tag to the lymphoproliferative element.

In some embodiments, the lymphoproliferative element can include a transmembrane domain. In some embodiments, the transmembrane domain can include a transmembrane domain from BAFFR, C3Z, CEACAM1, CD2, CD3A, CD3B, CD3D, CD3E, CD3G, CD3Z, CD4, CD5, CD7, CD8A, CD8B, CD9, CD11A, CD11B, CD11C, CD11D, CD27, CD16, CD18, CD19, CD22, CD28, CD29, CD33, CD37, CD40, CD45, CD49A, CD49D, CD49F, CD64, CD79A, CD79B, CD80, CD84, CD86, CD96 (Tactile), CD100 (SEMA4D), CD103, C134, CD137, CD154, CD160 (BY55), CD162 (SELPLG), CD226 (DNAM1), CD229 (Ly9), CD247, CRLF2, CRTAM, CSF2RA, CSF2RB, CSF3R, EPOR, FCER1G, FCGR2C, FCGRA2, GHR, HVEM (LIGHTR), IA4, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL7RA Ins PPCL, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA1, ITGA4, ITGA6, ITGAD, ITGAE, ITGAL, ITGAM, ITGAX, ITGB1, ITGB2, ITGB7, KIRDS2, LEPR, LFA-1 (CD11a, CD18), LIFR, LTBR, MPL, NKp80 (KLRF1), OSMR, PAG/Cbp, PRLR, PSGL1, SLAM (SLAMF1, CD150, IPO-3), SLAMF4 (CD244, 2B4), SLAMF6 (NTB-A, Ly108), SLAMF7, SLAMF8 (BLAME), TNFR2, TNFRSF4, TNFRSF8, TNFRSF9, TNFRSF14, TNFRSF18, VLA1, or VLA-6, or functional mutants and/or fragments thereof.

CLEs for use in any aspect or embodiment herein can include any CLE disclosed in WO2019/055946 (incorporated by reference herein, in its entirety), the vast majority of which were designed to be and are believed to be constitutively active typically because they constitutively activate a signaling pathway, typically through functional domains on their intracellular domains. In some embodiments, the constitutively active signaling pathways include activation of a Jak pathway, a Stat pathway, or Jak/Stat pathways including Jak1, Jak2, Jak3, and Tyk2 and STATs such as STAT1, STAT2, STAT3, STAT4, STAT5, STAT6, and in illustrative embodiments, STAT3 and/or STAT5. Accordingly, provided herein in certain embodiments, are lymphoproliferative elements that comprises a means for activating any one or more of these pathways, and typically an intracellular domain that is a means for activating any one or more of these pathways. In certain embodiments, lymphoproliferative elements comprise a means, such as an intracellular domain that is a means, for transmitting a signal that promotes proliferation of a T cell and/or NK cell, in illustrative embodiments when part of a dimerized lymphoproliferative element. In some embodiments, a CLE includes one or more STAT-activation domains. In some embodiments, a CLE includes two or more, three or more, four or more, five or more, or six or more STAT-activation domains. In some embodiments, at least one of the one or more STAT-activation domains is, or is derived from BLNK, IL2RG, EGFR, EpoR, GHR, IFNAR1, IFNAR2, IFNAR½, IFNLR1, IL10R1, IL12Rb1, IL12Rb2, IL21R, IL2Rb, IL2small, IL7R, IL7Ra, IL9R, IL15R, and IL21R, as are known in the art. In some embodiments, two or more STAT-activation domains are, or are derived from two or more different receptors. In some embodiments, the constitutively active signaling pathways include activation of a TRAF pathway through activation of TNF receptor associated factors such as TRAF3, TRAF4, TRAF7, and in illustrative embodiments TRAF1, TRAF2, TRAF5, and/or TRAF6. Thus, in certain embodiments, lymphoproliferative elements for use in any of the kits, methods, uses, or compositions herein, are constitutively active and comprise an intracellular signaling domain that activates a Jak/Stat pathway and/or a TRAF pathway. In some embodiments, the constitutively active signaling pathways include activation of PI3K pathways. In some embodiments, the constitutively active signaling pathways include activation of PLC pathways. Thus, in certain embodiments, lymphoproliferative elements for use in any of the kits, methods, uses, or compositions herein, are constitutively active and comprise an intracellular signaling domain that activates a Jak/Stat pathway a TRAF pathway, a PI3K pathway, and/or a PLC pathway. As illustrated therein, where there is a first and a second intracellular signaling domain of a CLE, the first intracellular signaling domain is positioned between the membrane associating motif, for example, a transmembrane domain, and the second intracellular domain.

In some embodiments, the lymphoproliferative elements provided herein include one or more, or all of the binding domains, including those disclosed herein, responsible for signaling found in the corresponding lymphoproliferative element in nature. In some embodiments, the lymphoproliferative elements provided herein include one or more JAK binding domains. In some embodiments, the JAK-binding domain is, or is derived from, EPOR, GP130, PRLR, GHR, GCSFR, or TPOR/MPLR. JAK-binding domains from these proteins are known in the art and a skilled artisan will understand how to use them. For example, residues 273-338 of EpoR and residues 478-582 of TpoR are known to be JAK-binding domains. Conserved motifs that are found in intracellular domains of cytokine receptors that are responsible for this signaling are known and are present in certain illustrative lymphoproliferative elements provided herein (see e.g., Morris et al., “The molecular details of cytokine signaling via the JAK/STAT pathway,” Protein Science (2018) 27:1984-2009). The Box1 and Box2 motifs are involved in binding to JAKs and signal transduction, although the Box2 motif presence is not always required for a proliferative signal (Murakami et al. Proc Natl Acad Sci U S A. 1991 Dec 15; 88(24):11349-53; Fukunaga et al. EMBO J. 1991 Oct; 10(10):2855-65; and O’Neal and Lee. Lymphokine Cytokine Res. 1993 Oct; 12(5):309-12). Accordingly, in some embodiments a lymphoproliferative element herein is a transgenic Box1-containing cytokine receptor that includes an intracellular domain of a cytokine receptor comprising a Box1 Janus kinase (JAK)-binding motif, optionally a Box2 JAK-binding motif, and a Signal Transducer and Activator of Transcription (STAT) binding motif comprising a tyrosine residue. In some embodiments, a lymphoproliferative element includes two or more JAK-binding motifs, for example three or more or four or more JAK-binding motifs, which in illustrative are the binding motifs found in natural versions of the corresponding lymphoproliferative element. In some embodiments, a lymphoproliferative element comprises an intracellular domain that is a means for transmitting a signal that promotes proliferation of a T cell and/or NK cell when part of a dimerized lymphoproliferative element

Intracellular domains from IFNAR1, IFNGR1, IFNLR1, IL2RB, IL4R, IL5RB, IL6R, IL6ST, IL7RA, IL9R, IL10RA, IL21R, IL27R, IL31RA, LIFR, and OSMR are known in the art to activate JAK1 signaling and thus comprise a JAK1 binding motif. Intracellular domains from CRLF2, CSF2RA, CSF2RB, CSF3R, EPOR, GHR, IFNGR2, IL3RA, IL5RA, IL6ST, IL20RA, IL20RB, IL23R, IL27R, LEPR, MPL, and PRLR are known in the art to activate JAK2 and thus comprise a JAK2 binding motif. Intracellular domains from IL2RG are known in the art to activate JAK3 and thus comprise a JAK3 binding motif. Intracellular domains from GHR, IFNAR1, IFNAR2, IFNGR1, IFNGR2, IL2RB, IL2RG, IL4R, IL5RA, IL5RB, IL7RA, IL9R, IL21R, IL22RA1, IL31RA, LIFR, MPL, and OSMR are known in the art to activate STAT1. Intracellular domains from IFNAR1 and IFNAR2 are known in the art to activate STAT2. Intracellular domains from GHR, IL2RB, IL2RG, IL6R, IL7RA, IL9R, IL10RA, IL10RB, IL21R, IL22RA1, IL23R, IL27R, IL31RA, LEPR, LIFR, MPL, and OSMR are known in the art to activate STAT3. Intracellular domains from IL12RB1 are known in the art to activate STAT4. Intracellular domains from CSF2RA, CSF2RB, CSF3R, EPOR, GHR, IL2RB, IL2RG, IL3RA, IL4R, IL5RA, IL5RB, IL7RA, IL9R, IL15RA, IL20RA, IL20RB, IL21R, IL22RA1, IL31RA, LIFR, MPL, OSMR, and PRLR are known in the art to activate STAT5. Intracellular domains from IL4R and OSMR are known in the art to activate STAT6. The genes and intracellular domains thereof that are found in a first intracellular domain are the same as the optional second intracellular domain, except that if the first and second intracellular domain are identical, then at least one, and typically both the transmembrane domain and the extracellular domain are not from the same gene.

In some embodiments, a lymphoproliferative element herein can include one or more intracellular signaling domains that include one or more Box1 motifs. In some embodiments, the one or more intracellular signaling domains that include one or more Box1 motifs can be IL7RA (Box1 motif at residues 9-17 of SEQ ID NOs:248 and 249), IL12RB ((Box1 motifs at residues 10-12 of SEQ ID NOs:254 and 255; and residues 107-110 and 139-142 of SEQ ID NO:256), IL31RA (Box1 motifs at residues 12-15 of SEQ ID NOs:275 and 276), CSF2RB (Box1 motif at residues 14-22 of SEQ ID NO:213), IL2RB (Box1 motif at residues 13-21 of SEQ ID NO:240), IL6ST (Box1 motif at residues 10-18 of SEQ ID NO:247), IL2RG (Box 1 motif at residues 3-11 of SEQ ID NO:241), IL27RA (Box1 motif at residues 17-25 of SEQ ID NO:273), MPL (Box1 motif at residues 17-20 of SEQ ID NO:283), OSMR (Box1 motif at residues 16-30 of SEQ ID NO:294), IFNAR2 (Box1 motif at residues 23-31 of SEQ ID NO:227), CSF3R, or EPOR (Box1 motif at residues 257-264 of full-length EPOR).

In some embodiments, a lymphoproliferative element herein can include one or more intracellular signaling domains that include one or more Box2 motifs. In some embodiments, the one or more intracellular signaling domains that include one or more Box2 motifs can be MPL (Box2 motif at residues 46-64 in SEQ ID NO:283), IFNAR2 (Box1 motif at residues 37-46 of SEQ ID NO:227), CSF3R, or EPOR (Box2 motif at residues 303-313 of full-length EPOR). EPOR also contains an extended Box2 motif (residues 329-372 of full-length EPOR) important for binding tyrosine kinase receptor KIT, which, in some embodiments, a lymphoproliferative element can include. CSF3R also contains a Box3 motif, which, in some embodiments, a lymphoproliferative element can include.

Some intracellular signaling domains have hydrophobic residues at positions -1, -2, and -6 relative to the Box1 motif, that form a “switch motif,” which is required for cytokine-induced JAK2 activation but not for JAK2 binding (Constantinescu et al. Mol Cell. 2001 Feb; 7(2):377-85; and Huang et al. Mol Cell. 2001 Dec; 8(6):1327-38). Accordingly, in certain embodiments, the Box1 motif-containing lymphoproliferative element has a switch motif, which in illustrative embodiments has one or more, and preferably all hydrophobic residues at positions -1, -2, and -6 relative to the Box1 motif. In certain embodiments, the Box1 motif an ICD of a lymphoproliferative element is located proximal to the transmembrane (TM) domain (for example between 5 and 15 or about 10 residues downstream from the TM domain) relative to the Box2 motif, which is located proximal to the transmembrane domain (for example between 10 and 50 residues downstream from the TM domain) relative to the STAT binding motif. The STAT binding motif typically comprising a tyrosine residue, the phosphorylation of which affects binding of a STAT to the STAT binding motif of the lymphoproliferative element. In some embodiments, the ICDs comprising multiple STAT binding motifs where multiple STAT binding motifs are present in a native ICD (e.g., EPO receptor and IL-6 receptor signaling chain (gp130). In some embodiments, the switch motif containing intracellular signaling domain can be MPL (switch motif at residues 11, 15, and 16 of SEQ ID NO:283).

In some embodiments, a lymphoproliferative element herein can include one or more intracellular signaling domains that include one or more phosphorylatable residues, for example, a phosphorylatable serine, threonine, or tyrosine. In some embodiments, the one or more intracellular signaling domains that include one or more phosphorylatable residues can be IL31RA (phosphorylatable tyrosines at residues Y96, Y237, and Y165 of SEQ ID NO:275; not present in SEQ ID NO:276), CD27 (phosphorylatable serine at residue S6 of SEQ ID NO:205), CSF2RB (phosphorylatable tyrosine at residue Y306 of SEQ ID NO:213), IL6ST (phosphorylatable serines at residues S20, S26, S141, S148, S188, and S198 of SEQ ID NO:247), MPL (phosphorylatable tyrosines at residues Y8, Y29, Y78, Y113, and Y118 of SEQ ID NO: 283), CD79B (phosphorylatable tyrosines at residues Y16 and Y27 of SEQ ID NO: 211), OSMR (phosphorylatable serines at residues S65 and S128 of SEQ ID NO:294), or CD3G (phosphorylatable serines at residues S123 and S126 of full-length CD3G). In some embodiments, a lymphoproliferative element that includes a CSF3R intracellular domain can include one, two, three, or all of the tyrosine residues corresponding to Y704, Y729, Y744, and Y764 of full-length CSF3R, various combinations of which have been shown to be important for binding Stat3, SOCS3, Grb2, and p21Ras. In some embodiments, a lymphoproliferative element herein can include one or more intracellular signaling domains that has one or more of its phosphorylatable residues mutated to a phosphomimetic residue, for example, aspartic acid or glutamic acid. In some embodiments, a lymphoproliferative element herein can include one or more intracellular signaling domains that has one or more of its phosphorylatable tyrosines mutated to a non-phosphorylatable residue, for example, alanine, valine, or phenylalanine. In some embodiments, a lymphoproliferative element that includes a CSF3R intracellular domain can include one or more mutations corresponding to T615A and T618I of full-length CSF3R, which have been shown to increase receptor dimerization and activity.

In some embodiments, a lymphoproliferative element herein can include one or more intracellular signaling domains that include one or more ubiquitination targeting motif residues. In some embodiments, the one or more intracellular signaling domains that include one or more ubiquitination targeting motif residues can be MPL (residues at K40 and K60 of SEQ ID NO:283) or OX40 (residues at K17 and K41 of SEQ ID NO:296). In some embodiments herein, an intracellular domain including ubiquitination targeting motif residues can have one or more of the lysines mutated to arginine or another amino acid.

In some embodiments, a lymphoproliferative element herein can include one or more intracellular signaling domains that include one or more TRAF binding sites. Not to be limited by theory, TRAF1, TRAF2, and TRAF3 binding sites include the amino acid sequence PXQXT (SEQ ID NO:303), where each X can be any amino acid, a distinct TRAF2 binding site includes the consensus sequence SXXE (SEQ ID NO:304) where each X can be any amino acid, and a TRAF6 binding site includes the consensus sequence QXPXEX (SEQ ID NO:305). In some embodiments, the one or more intracellular signaling domains that include one or more TRAF binding sites can be CD40 (binding sites for TRAF1, TRAF2, and TRAF3 at residues 35-39 of SEQ ID NO:208; TRAF2 binding site at residues 57-60 of SEQ ID NO:208; TRAF6 binding site at residues 16-21 of SEQ ID NO:208), or OX40 (TRAF1, TRAF2, TRAF3, and TRAF5 binding motif at residues 20-27 of SEQ ID NO:296).

In some embodiments, a lymphoproliferative element herein can include one or more intracellular signaling domains that include a TIR domain. In some embodiments, the one or more intracellular signaling domains that include a TIR domains can be IL17RE (TIR domain at residues 13-136 of SEQ ID NO:265), IL18R1 (TIR domain at residues 28-170 of SEQ ID NO:266), or MyD88 (TIR domain at residues 160-304 of SEQ ID NO:284).

In some embodiments, a lymphoproliferative element herein can include one or more intracellular signaling domains that include a PI3K binding motif domain. In some embodiments, the one or more intracellular signaling domains that include a PI3K binding motif can be CD28 (PI3K binding motifs at residues 12-15 of SEQ ID NOs:206 and 207, which also binds Grb2), ICOS (PI3K binding motif at residues 19-22 of SEQ ID NO:225, which can be mutated F21Q to increase IL-2 production and/or to bind Grb2), OX40 (p85 PI3K binding motif at residues 34-57 of full-length OX40)

In some embodiments, a lymphoproliferative element herein can include one or more intracellular signaling domains that include a dileucine motif. In some embodiments, the one or more intracellular signaling domains that include a dileucine motif can be IFNGR2 (dileucine motif at residues 8-9 of SEQ ID NO:230) or CD3G (dileucine motif at residues 131-132 of full-length CD3G). In some embodiments, one or both of the residues in the dileucine motif can be mutated.

In some embodiments, a lymphoproliferative element herein can include one or more intracellular signaling domains that include one or more N-terminal death domains. In some embodiments, the one or more intracellular signaling domains that include one or more N-terminal death domains can be MyD88 (N-terminal death domain at residues 29-106 of SEQ ID NO:284) or a TNFR. The cytoplasmic domains of TNF receptors (TNFRs), which in illustrative embodiments can be TNFRSF4, TNFRSF8, TNFRSF9, TNFRSF14, or TNFRSF18, can recruit signaling molecules, including TRAFs (TNF receptor-associated factors) and/or “death domain” (DD) molecules. The domains, motifs, and point mutations of TNFRs that induce proliferation and/or survival of T cells and/or NK cells are known in the art and a skilled artisan can identify corresponding domains, motifs, and point mutations in TNFR polypeptides. A skilled artisan will be able to identify the TRAF- and/or DD-binding motif in the different TNFR families using, for example, sequence alignments to known binding motifs. In some embodiments, a lymphoproliferative element that includes a TNFR intracellular domain can include one or more TRAF-binding motifs. In some embodiments, a lymphoproliferative element that includes a TNFR intracellular domain does not include a DD-binding motif, or has one or more DD-binding motifs deleted or mutated within the intracellular domain. In some embodiments, a lymphoproliferative element that includes a TNFR intracellular domain can recruit TRADD and/or TRAF2. TNFRs also include cysteine-rich domains (CRDs) that are important for ligand binding (Locksley RM et al. Cell. 2001 Feb 23;104(4):487-501). In some embodiments, a lymphoproliferative element that includes a TNFR intracellular domain does not include a TNFR CRD.

In some embodiments, a lymphoproliferative element herein can include one or more intracellular signaling domains that include one or more intermediate domains that interact with IL-1R associated kinase. In some embodiments, the one or more intracellular signaling domains that include one or more intermediate domains can be MyD88 (intermediate domain at residues 107-156 of SEQ ID NO:284),

In some embodiments, a lymphoproliferative element that includes an intracellular domain from IL7RA can include one or more of the S region or T region (S region at residues 359-394 and T region at residues Y401, Y449, and Y456 of full-length IL7RA). In illustrative embodiments of lymphoproliferative elements that include a first intracellular domain derived from IL7RA, the second intracellular domain can be derived from TNFRSF8.

In illustrative embodiments of lymphoproliferative elements that include a first intracellular domain derived from CD40, the second intracellular domain can be other than an intracellular domain derived from MyD88, a CD28 family member (e.g., CD28, ICOS), Pattern Recognition Receptor, a C-reactive protein receptor (i.e., Nodi, Nod2, PtX3-R), a TNF receptor, CD40, RANK/TRANCE-R, OX40, 4-1BB), an HSP receptor (Lox-1 and CD91), or CD28. Pattern Recognition Receptors include, but are not limited to endocytic pattern-recognition receptors (i.e., mannose receptors, scavenger receptors (i.e., Mac-1, LRP, peptidoglycan, teichoic acids, toxins, CD1 1 c/CR4)); external signal pattern-recognition receptors (Toll-like receptors (TLR1, TLR2, TLR3, TLR4, TLR5, TLR6, TLR7, TLR8, TLR9, TLR10), peptidoglycan recognition protein, (PGRPs bind bacterial peptidoglycan, and CD14); internal signal pattern-recognition receptors (i.e., NOD-receptors 1 & 2), and RIG1.

In some embodiments, a lymphoproliferative element that includes an intracellular domain from MyD88 can include one or more of the mutations L93P, R193C, and L265P in full-length MyD88 (mutations L93P, R196C, and L260P of SEQ ID NO:284). In illustrative embodiments of lymphoproliferative elements that include a first intracellular domain derived from MyD88, the second intracellular domain can be derived from TNFRSF4 or TNFRSF8. In other illustrative embodiments of lymphoproliferative elements that include a first intracellular domain derived from MyD88, the second intracellular domain can be other than an intracellular domain derived from a CD28 family member (e.g., CD28, ICOS), Pattern Recognition Receptor, a C-reactive protein receptor, a TNF receptor, or an HSP receptor.

In some embodiments, a cell expressing the lymphoproliferative element comprising an intracellular and transmembrane domain of MPL can be contacted with or exposed to eltrombopag, or a patient or subject to which such a cell has been infused can be treated with eltrombopag. Not to be limited by theory, eltrombopag binds to the transmembrane domain of MPL and induces the activation of the intracellular domain of MPL.

The domains, motifs, and point mutations of MPL that induce proliferation and/or survival of T cells and/or NK cells are known in the art and a skilled artisan can identify corresponding domains, motifs, and point mutations in MPL polypeptides, some of which are discussed in this paragraph. Deletion of the region encompassing amino acids 70-95 in SEQ ID NO:283 was shown to support viral transformation in the context of v-mpl (Benit et al. J Virol. 1994 Aug; 68(8):5270-4), thus indicating that this region is not necessary for the function of mpl in this context. Morello et al. Blood 1995 July; 86(8):557-71 used the same deletion to show that this region was not required for stimulating transcription for a hematopoietin receptor-responsive CAT reporter gene construct and furthermore saw that this deletion resulted in slightly enhanced transcription expected for removal of a nonessential and negative element in this region as suggested by Drachman and Kaushansky. Thus, in some embodiments, a MPL intracellular signaling domain does not comprise the region comprising amino acids 70-95 in SEQ ID NO:283. Using computer simulations, Lee et al. found clinically relevant mutations in the transmembrane domain of MPL should activate MPL with the following order of activating effects: W515K (corresponding to the amino acid substitution W2K of SEQ ID NO: 283) > S505A (corresponding to the amino acid substitution S14A of SEQ ID NO:187) > W515I (corresponding to the amino acid substitution W2I of SEQ ID NO: 283) > S505N (corresponding to the amino acid substitution S14N of SEQ ID NO:187, which was tested as part T075 (SEQ ID NO:188)) (Lee et. al. PLoS One. 2011; 6(8): e23396). The simulations predicted these mutations could cause constitutive activation of JAK2, the kinase partner of MPL. In some embodiments, the intracellular portion of MPL can include one or more, or all the domains and motifs described herein that are present in SEQ ID NO: 283. In some embodiments, a transmembrane portion of MPL can include one or more, or all the domains and motifs described herein that are present in SEQ ID NO:187. In illustrative embodiments of lymphoproliferative elements that include a first intracellular domain derived from MPL, the second intracellular domain can be derived from CD79B.

In illustrative embodiments of lymphoproliferative elements that include a second intracellular domain derived from CD79B, the first intracellular domain can be derived from CSF3R.

In some embodiments, a lymphoproliferative element that includes an PRLR intracellular domain can include the growth hormone receptor binding domain of PRLR and any known mutations (growth hormone receptor binding domain at residues 28-104 of SEQ ID NO:295).

In some embodiments, a lymphoproliferative element that includes an ICOS intracellular domain can include a calcium-signaling motif (calcium-signaling motif at residues 5-8 of SEQ ID NO:225). In some embodiments, a lymphoproliferative element that includes an ICOS intracellular domain can include at least one of a first and second conserved motif (first and second conserved motifs at residues 9-18 and 24-30, respectively, of SEQ ID NO:225). In some embodiments, a lymphoproliferative element that includes an ICOS intracellular domain does not include at least one of the first and second conserved motif.

EPOR also contains a short segment important for EPOR internalization (residues 267-276 of full-length EPOR). In some embodiments, a lymphoproliferative element that includes an EPOR intracellular domain does not include the internalization segment.

The domains, motifs, and point mutations of intracellular signaling domains that induce proliferation and/or survival of T cells and/or NK cells are known in the art and a skilled artisan can identify corresponding domains, motifs, and point mutations in polypeptides, some of which are above, and a skilled artisan can identify corresponding domains, motifs, and point mutations in other polypeptides. A skilled artisan will be able to identify these domains, motifs, and point mutations in similar polypeptides using, for example, sequence alignments to known binding motifs. In some embodiments, a lymphoproliferative element herein can include any, for example, one or more up to all of the domains, motifs, and mutations of a intracellular signaling domain disclosed herein or otherwise known to induce proliferation and/or survival of T cells and/or NK cells.

In another embodiment, the LE provides, is capable of providing and/or possesses the property of (or a cell modified, genetically modified, and/or transduced with the LE is capable of providing, is adapted for, possesses the property of, and/or is modified for) driving T cell expansion in vivo.

In some embodiments, the lymphoproliferative element can include any of the sequences listed in Table 1 (SEQ ID NOs: 84-302). Table 1 shows the parts, names (including gene names), and amino acid sequences for domains that were tested in CLEs. CLEs can include in certain illustrative embodiments, an extracellular domain (denoted P1), a transmembrane domain (denoted P2), a first intracellular domain (denoted P3), and a second intracellular domain (denoted P4). Typically, the lymphoproliferative element includes a first intracellular domain. In illustrative embodiments, the first intracellular domain can include any of the parts listed as S036 to S0216 or in Table 1, or functional mutants and/or fragments thereof. In some embodiments, the lymphoproliferative element can include a second intracellular domain. In illustrative embodiments, the second intracellular domain can include any of the parts listed as S036 to S0216 or in Table 1, or functional mutants and/or fragments thereof. In some embodiments, the lymphoproliferative element can include an extracellular domain. In illustrative embodiments, the extracellular domain can include any of the sequences of parts listed as M001 to M049 or E006 to E015 in Table 1, or functional mutants and/or fragments thereof. In some embodiments, the lymphoproliferative element can include a transmembrane domain. In illustrative embodiments, the transmembrane domain can include any of the parts listed as M001 to M049 or T001 to T082 in Table 1, or functional mutants and/or fragments thereof. In some embodiments, the lymphoproliferative element can be a fusion of an extracellular/transmembrane domain (M001 to M049 in Table 1), a first intracellular domain (S036 to S0216 in Table 1), and a second intracellular domain (S036 to S216 in Table 1). In some embodiments, the lymphoproliferative element can be a fusion of an extracellular domain (E006 to E016 in Table 1), a transmembrane domain (T001 to T082 in Table 1), a first intracellular domain (S036 to S0216 in Table 1), and a second intracellular domain (S036 to S0216 in Table 1). For example, the lymphoproliferative element can be a fusion of E006, T001, S036, and S216, also written as E006-T001-S036-S216). In illustrative embodiments, the lymphoproliferative element can be the fusion E010-T072-S192-S212, E007-T054-S197-S212, E006-T006-S194-S211, E009-T073-S062-S053, E008-T001-S121-S212, E006-T044-S186-S053, or E006-T016-S186-S050.

In illustrative embodiments, the intracellular domain of an LE, or the first intracellular domain in an LE that has two or more intracellular domains, is other than a functional intracellular activating domain from an ITAM-containing intracellular domain, for example, an intracellular domain from CD3Z, CD3D, CD3E, CD3G, CD79A, CD79B, DAP12, FCER1G, FCGR2A, FCGR2C, DAP10/CD28, or ZAP70, and in a further illustrative subembodiment, CD3z. In illustrative embodiments, the extracellular domain of an LE does not comprise a single-chain variable fragment (scFv). In further illustrative embodiments, the extracellular domain of an LE that upon binding to a binding partner activates an LE, does not comprise a single-chain variable fragment (scFv). A CLE does not comprise both an ASTR and an activation domain from CD3Z, CD3D, CD3E, CD3G, CD79A, CD79B, DAP12, FCER1G, FCGR2A, FCGR2C, DAP10/CD28, or ZAP70. If an LE does include an ASTR (and not an activation domain in the previous list), the ASTR of an LE in illustrative embodiments does not include an scFv. In some embodiments, a lymphoproliferative element does not include an extracellular domain.

In some embodiments, the lymphoproliferative element, and in illustrative embodiments CLE, is not covalently attached to a cytokine. In some aspects, a lymphoproliferative element, and in illustrative embodiments CLE, comprises a cytokine polypeptide covalently linked to its cognate receptor. In either of these embodiments, the CLE can be constitutively active and typically constitutively activates the same Jak/STAT and/or TRAF pathways as the corresponding activated wild-type cytokine receptor. In some embodiments, the chimeric cytokine receptor is an interleukin. In some embodiments, the CLE is IL-7 covalently linked to IL7RA or IL-15 covalently linked to IL15RA. In other embodiments, the CLE is other than IL-15 covalently linked to IL15RA. In other aspects, the CLE comprises a cytokine polypeptide covalently linked to only a portion of its cognate receptor that includes a functional portion of the extracellular domain capable of binding the cytokine polypeptide, the transmembrane domain and/or intracellular domain are from heterologous polypeptides, and the CLE is constitutively active. In one embodiment, the CLE is IL-7 covalently linked to the extracellular and transmembrane domains of IL7RA, and the intracellular domain from IL2RB. In another embodiment, the CLE is a cytokine polypeptide covalently linked to a portion of its cognate receptor that includes a functional portion of the extracellular domain capable of binding the cytokine polypeptide, a heterologous transmembrane domain, and lymphoproliferative element intracellular domain provided herein. In some embodiments, the lymphoproliferative element is a cytokine receptor that is not tethered to a cytokine.

In some aspects, the lymphoproliferative element is capable of binding to soluble cytokines or growth factors and such binding is required for activity. In certain illustrative embodiments, the lymphoproliferative element is constitutively active, and thus does not require binding to a soluble growth factor or cytokine for activity. Typically, constitutively active lymphoproliferative elements do not bind soluble cytokines or growth factors. In some embodiments, the lymphoproliferative element is a chimera comprising an extracellular binding domain from one receptor and the intracellular signaling domain from a different receptor. In some embodiments the CLE is an inverted receptor that is activated upon binding of a ligand that would inhibit proliferation and/or survival when bound to its natural receptor, but instead leads to proliferation and/or survival upon activating the CLE. In some embodiments, inverted receptors include chimeras that comprise an extracellular ligand binding domain from IL4Ra and an intracellular domain from IL7Ra or IL21. Other embodiments of inverted cytokine receptors include chimeras that comprise an extracellular ligand binding domain from a receptor that would inhibit proliferation and/or survival when bound to its natural ligand, such as receptors for IL-4, IL-10, IL-13, or TGFb, and any lymphoproliferative element intracellular domain disclosed herein. In illustrative aspects, the lymphoproliferative element does not bind a cytokine. In further illustrative aspects, the lymphoproliferative element does not bind any ligand. In illustrative embodiments, the lymphoproliferative elements that do not bind any ligand are constitutively dimerized or otherwise multimerized, and are constitutively active. In illustrative embodiments of any of the methods and compositions provided herein that include a lymphoproliferative element, the intracellular domain can be derived from an intracellular portion of the transmembrane protein of the TNF receptor family, CD40. The domains, motifs, and point mutations of CD40 that induce proliferation and/or survival of T cells and/or NK cells are known in the art and a skilled artisan can identify corresponding domains, motifs, and point mutations in CD40 polypeptides, some of which are discussed in this paragraph. The CD40 protein contains several binding sites for TRAF proteins. Not to be limited by theory, binding sites for TRAF1, TRAF2, and TRAF3 are located at the membrane distal domain of the intracellular portion of CD40 and include the amino acid sequence PXQXT (SEQ ID NO:303) where each X can be any amino acid, (corresponding to amino acids 35-39 of SEQ ID NO:208) (Elgueta et al. Immunol Rev. 2009 May; 229(1):152-72). TRAF2 has also been shown to bind to the consensus sequence SXXE (SEQ ID NO:304) where each X can be any amino acid, (corresponding to amino acids 57-60 of SEQ ID NO:208) (Elgueta et al. Immunol Rev. 2009 May; 229(1):152-72). A distinct binding site for TRAF6 is situated at the membrane proximal domain of intracellular portion of CD40 and includes the consensus sequence QXPXEX (SEQ ID NO:305) where each X can be any amino acid (corresponding to amino acids 16-21 of SEQ ID NO:208) (Lu et al. J Biol Chem. 2003 Nov 14; 278(46):45414-8). In illustrative embodiments, the intracellular portion of the transmembrane protein CD40 can include all the binding sites for the TRAF proteins. The TRAF binding sites are known in the art and a skilled artisan will be able to identify corresponding TRAF binding sites in similar CD40 polypeptides. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:208 or SEQ ID NO:209. In some embodiments, the intracellular domain derived from CD40 has a length of from about 30 amino acids (aa) to about 35 aa, from about 35 aa to about 40 aa, from about 40 aa to about 45 aa, from about 45 aa to about 50 aa, from about 50 aa to about 55 aa, from about 55 aa to about 60 aa, or from about 60 aa to about 65 aa. In illustrative embodiments, the intracellular domain derived from CD40 has a length of from about 30 aa to about 66 aa, for example, 30 aa to 65 aa, or 50 aa to 66 aa. In illustrative embodiments of lymphoproliferative elements that include a first intracellular domain derived from CD40, the second intracellular domain can be other than an intracellular domain derived from MyD88, a CD28 family member (e.g., CD28, ICOS), Pattern Recognition Receptor, a C-reactive protein receptor (i.e., Nodi, Nod2, PtX3-R), a TNF receptor, CD40, RANK/TRANCE-R, OX40, 4-1BB), an HSP receptor (Lox-1 and CD91), or CD28. Pattern Recognition Receptors include, but are not limited to endocytic pattern-recognition receptors (i.e., mannose receptors, scavenger receptors (i.e., Mac-1, LRP, peptidoglycan, teichoic acids, toxins, CD1 1 c/CR4)); external signal pattern-recognition receptors (Toll-like receptors (TLR1, TLR2, TLR3, TLR4, TLR5, TLR6, TLR7, TLR8, TLR9, TLR10), peptidoglycan recognition protein, (PGRPs bind bacterial peptidoglycan, and CD14); internal signal pattern-recognition receptors (i.e., NOD-receptors 1 & 2), and RIG1.

In illustrative embodiments of any of the methods and compositions provided herein that include a lymphoproliferative element, the intracellular domain can be derived from a portion of the transmembrane protein MPL. Accordingly, in some embodiments, the lymphoproliferative element comprises MPL, or is MPL, or a variant and/or fragment thereof, including a variant and/or fragment that includes at least 75, 80, 85, 90, 95, 96, 97, 98, 99, or 100% of the intracellular domain of MPL, with or without a transmembrane and/or extracellular domain of MPL, wherein the variant and/or fragment retains the ability to promote cell proliferation of PBMCs, and in some embodiments T cells. In some embodiments, a cell expressing the lymphoproliferative element comprising an intracellular and transmembrane domain of MPL can be contacted with, exposed to, or treated with eltrombopag. Not to be limited by theory, eltrombopag binds to the transmembrane domain of MPL and induces the activation of the intracellular domain of MPL. The domains, motifs, and point mutations of MPL that induce proliferation and/or survival of T cells and/or NK cells are known in the art and a skilled artisan can identify corresponding domains, motifs, and point mutations in MPL polypeptides, some of which are discussed in this paragraph. The transmembrane MPL protein contains the Box1 motif PXXP (SEQ ID NO:306) where each X can be any amino acid (corresponding to amino acids 17-20 in SEQ ID NO:283) and the Box2 motif, a region with increased serine and glutamic acid content (corresponding to amino acids 46-64 in SEQ ID NO:283) (Drachman and Kaushansky. Proc Natl Acad Sci U S A. 1997 Mar 18; 94(6):2350-5). The Box1 and Box2 motifs are involved in binding to JAKs and signal transduction, although the Box2 motif presence is not always required for a proliferative signal (Murakami et al. Proc Natl Acad Sci U S A. 1991 Dec 15; 88(24):11349-53; Fukunaga et al. EMBO J. 1991 Oct; 10(10):2855-65; and O’Neal and Lee. Lymphokine Cytokine Res. 1993 Oct; 12(5):309-12). Many cytokine receptors have hydrophobic residues at positions -1, -2, and -6 relative to the Box1 motif (corresponding to amino acids 16, 15, and 11, respectively, of SEQ ID NO:283), that form a “switch motif,” which is required for cytokine-induced JAK2 activation but not for JAK2 binding (Constantinescu et al. Mol Cell. 2001 Feb; 7(2):377-85; and Huang et al. Mol Cell. 2001 Dec; 8(6):1327-38). Deletion of the region encompassing amino acids 70-95 in SEQ ID NO:283was shown to support viral transformation in the context of v-mpl (Benit et al. J Virol. 1994 Aug; 68(8):5270-4), thus indicating that this region is not necessary for the function of mpl in this context. Morello et al. Blood 1995 July; 86(8):557-71 used the same deletion to show that this region was not required for stimulating transcription for a hematopoietin receptor-responsive CAT reporter gene construct and furthermore saw that this deletion resulted in slightly enhanced transcription expected for removal of a nonessential and negative element in this region as suggested by Drachman and Kaushansky. Thus, in some embodiments, a MPL intracellular signaling domain does not comprise the region comprising amino acids 70-95 in SEQ ID NO:283. In full-length MPL, the lysines K553 (corresponding to K40 of SEQ ID NO: 283) and K573 (corresponding to K60 of SEQ ID NO: 283) have been shown to be negative regulatory sites that function as part of a ubiquitination targeting motif (Saur et al. Blood 2010 Feb 11;115(6):1254-63). Thus, in some embodiments herein, a MPL intracellular signaling domain does not comprise these ubiquitination targeting motif residues. In full-length MPL, the tyrosines Y521 (corresponding to Y8 of SEQ ID NO: 283), Y542 (corresponding to Y29 of SEQ ID NO:283), Y591 (corresponding to Y78 of SEQ ID NO: 283), Y626 (corresponding to Y113 of SEQ ID NO: 283), and Y631 (corresponding to Y118 of SEQ ID NO: 283) have been shown to be phosphorylated (Varghese et al. Front Endocrinol (Lausanne). 2017 Mar 31; 8:59). Y521 and Y591 of full-length MPL are negative regulatory sites that function either as part of a lysosomal targeting motif (Y521) or via an interaction with adaptor protein AP2 (Y591) (Drachman and Kaushansky. Proc Natl Acad Sci U S A. 1997 Mar 18; 94(6):2350-5; and Hitchcock et al. Blood. 2008 Sep 15; 112(6):2222-31). Y626 and Y631 of full-length MPL are positive regulatory sites (Drachman and Kaushansky. Proc Natl Acad Sci U S A. 1997 Mar 18; 94(6):2350-5) and the murine homolog of Y626 is required for cellular differentiation and the phosphorylation of Shc (Alexander et al. EMBO J. 1996 Dec 2;15(23):6531-40) and Y626 is also required for constitutive signaling in MPL with the W515A mutation described below (Pecquet et al. Blood. 2010 Feb 4;115(5):1037-48). MPL contains the Shc phosphotyrosine-binding binding motif NXXY (SEQ ID NO:307) where each X can be any amino acid (corresponding to amino acids 110-113 of SEQ ID NO: 283), and this tyrosine is phosphorylated and important for the TPO-dependent phosphorylation of Shc, SHIP, and STAT3 (Laminet et al. J Biol Chem. 1996 Jan 5; 271(1):264-9; and van der Geer et al. Proc Natl Acad Sci USA. 1996 Feb 6; 93(3):963-8). MPL also contains the STAT3 consensus binding sequence YXXQ (SEQ ID NO:308) where each X can be any amino acid (corresponding to amino acids 118-121 of SEQ ID NO: 283) (Stahl et al. Science. 1995 Mar 3; 267(5202):1349-53). The tyrosine of this sequence can be phosphorylated and MPL is capable of partial STAT3 recruitment (Drachman and Kaushansky. Proc Natl Acad Sci U S A. 1997 Mar 18; 94(6):2350-5). MPL also contains the sequence YLPL (SEQ ID NO: 309) (corresponding to amino acid 113-116 of SEQ ID NO: 283), which is similar to the consensus binding site for STAT5 recruitment pYLXL (SEQ ID NO:310) where pY is phosphotyrosine and X can be any amino acid (May et al. FEBS Lett. 1996 Sep 30; 394(2):221-6). Using computer simulations, Lee et al. found clinically relevant mutations in the transmembrane domain of MPL should activate MPL with the following order of activating effects: W515K (corresponding to the amino acid substitution W2K of SEQ ID NO: 283) > S505A (corresponding to the amino acid substitution S14A of SEQ ID NO:187) > W515I (corresponding to the amino acid substitution W2I of SEQ ID NO: 283) > S505N (corresponding to the amino acid substitution S14N of SEQ ID NO:187, which was tested as part T075 (SEQ ID NO:188)) (Lee et. a. PLoS One. 2011; 6(8): e23396). The simulations predicted these mutations could cause constitutive activation of JAK2, the kinase partner of MPL. In some embodiments, the intracellular portion of MPL can include one or more, or all the domains and motifs described herein that are present in SEQ ID NO 283. In some embodiments, a transmembrane portion of MPL can include one or more, or all the domains and motifs described herein that are present in SEQ ID N0:187. The domains, motifs, and point mutations of MPL provided herein are known in the art and a skilled artisan would recognize that MPL intracellular signaling domains herein in illustrative embodiments would include one or more corresponding domains, motifs, and point mutations in that have been shown to promote proliferative activity and would not include that that have been shown to inhibit MPLs proliferative activity. Any or all of these domains, motifs, and point mutations of MPL can be present in an intracellular signaling domain can be included in any of the aspects and embodiments disclosed herein. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:283. In some embodiments, the intracellular domain derived from MPL has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 100 aa, from about 100 aa to about 125 aa, from about 125 aa to 150 aa, from about 150 to about 175 aa, from about 175 aa to about 200 aa, from about 200 aa to about 250 aa, from about 250 aa to 300 aa, from about 300 aa to 350 aa, from about 350 aa to about 400 aa, from about 400 aa to about 450 aa, from about 450 aa to about 500 aa, from about 500 aa to about 550 aa, from about 550 aa to about 600 aa, or from about 600 aa to about 635 aa. In illustrative embodiments, the intracellular domain derived from MPL has a length of from about 30 aa to about 200 aa, for example, 30 aa to 150 aa, 30 aa to 119 aa, 30 aa to 121 aa, 30 aa to 122 aa, or 50 aa to 125 aa. In illustrative embodiments of lymphoproliferative elements that include a first intracellular domain derived from MPL, the second intracellular domain can be derived from CD79B.

Lymphoproliferative elements and CLEs that can be included in any of the aspects disclosed herein, can be any of the LEs or CLEs disclosed in WO2019/055946. CLEs were disclosed therein that promoted proliferation in cell culture of PBMCs that were transduced with lentiviral particles encoding the CLEs between day 7 and day 21, 28, 35 and/or 42 after transduction. Furthermore, CLEs were identified therein, that promoted proliferation in vivo in mice in the presence or absence of an antigen recognized by a CAR, wherein T cells expressing one of the CLEs and the CAR were introduced into the mice. As exemplified therein, tests and/or criteria can be used to identify whether any test polypeptide, including LEs, or test domains of an LE, such as a first intracellular domain, or a second intracellular domain, or both a first and second intracellular domain, are indeed LEs or effective intracellular domains of LEs, or especially effective LEs or intracellular domains of LEs. Thus, in certain embodiments, any aspect or other embodiment provided herein that includes an LE or a polynucleotide or nucleic acid encoding an LE can recite that the LE meets, or provides the property of, or is capable of providing and/or possesses the property of, any one or more of the identified tests or criteria for identifying an LE provided herein, or that a cell genetically modified, transduced, and/or stably transfected with a recombinant nucleic acid vector, such as a cell that is transduced with a lentiviral particle encoding the LE, is capable of providing, is adapted for, possesses the property of, and/or is modified for achieving the results of one or more of the recited tests. In one embodiment, the LE provides, is capable of providing and/or possesses the property of, (or a cell genetically modified and/or transduced with a retroviral particle encoding the LE is capable of providing, is adapted for, possesses the property of, and/or is modified for) improved expansion to pre-activated PBMCs transduced with a lentivirus comprising a nucleic acid encoding the LE and an anti-CD19 CAR comprising a CD3 zeta intracellular activating domain but no co-stimulatory domain, between day 7 and day 21, 28, 35, and/or 42 of in vitro culturing post-transduction in the absence of exogenously added cytokines, compared to a control retroviral particle, e.g. lentiviral particle under identical conditions. In some embodiments, a lymphoproliferative element test for improved or enhanced survival, expansion, and/or proliferation of cells transduced with a retroviral particle (e.g. lentiviral particle) having a genome encoding a test construct encoding a putative LE (test cells) can be performed based on a comparison to control cells, which can be, for example, either untransduced cells or cells transduced with a control retroviral (e.g. lentiviral) particle identical to the lentiviral particle comprising the nucleic acid encoding the lymphoproliferative element, but lacking the lymphoproliferative element, or lacking the intracellular domain or domains of the test polypeptide construct but comprising the same extracellular domain, if present, and the same transmembrane region or membrane targeting region of the respective test polypeptide construct. In some embodiments control cells are transduced with a retroviral particle (e.g., lentiviral particle) having a genome encoding a lymphoproliferative element or intracellular domain(s) thereof, identified herein as exemplifying a lymphoproliferative element. In such an embodiment, the test criteria can include that there is at least as much enrichment, survival and/or expansion, or no statistical difference of enrichment, survival, and/or expansion when the test is performed using a retroviral particle (e.g., lentiviral particle) having a genome encoding a test construct versus encoding the control lymphoproliferative element, typically by analyzing cells transcribed therewith. Exemplary or illustrative embodiments of lymphoproliferative elements herein, in some embodiments, are illustrative embodiments of control lymphoproliferative elements for such a test.

In some embodiments, this test for an improved property of a putative or test lymphoproliferative element is performed by performing replicates and/or performing a statistical test. A skilled artisan will recognize that many statistical tests can be used for such a lymphoproliferative element test. Contemplated for such a test in these embodiments would be any such test known in the art. In some embodiments, the statistical test can be a T-test or a Mann-Whitney-Wilcoxon test. In some embodiments, the normalized enrichment level of a test construct is significant at a p-value of less than 0.1, or less than 0.05, or less than 0.01.

In another embodiment, the LE provides, is capable of providing and/or possesses the property of (or a cell genetically modified and/or transduced with the LE is capable of providing, is adapted for, possesses the property of, and/or is modified for) at least a 1.5-fold, 2-fold, 3-fold, 4-fold, 5-fold, 6-fold, 7-fold, 8-fold, 9-fold, or 10-fold expansion, or between 1.5 fold and 25-fold expansion, or between 2-fold and 20-fold expansion, or between 2-fold and 15-fold expansion, or between 5-fold and 25-fold expansion, or between 5-fold and 20-fold expansion, or between 5-fold and 15-fold expansion, of pre-activated PBMCs transduced with a nucleic acid encoding the LE when transduced along with an anti-CD19 CAR comprising a CD3 zeta intracellular activating domain but no co-stimulatory domain, between day 7 and day 21, 28, 35, and/or 42 of in vitro culturing in the absence of exogenously added cytokines. In some embodiments, the test is performed in the presence of PBMCs, for example at a 1:1 ratio of transduced cells to PBMCs, which can be for example, from a matched donor, and in some embodiments, the test is performed in the absence of PBMCs. In some embodiments, the analysis of expansion for any of these tests is performed as illustrated in WO2019/055946. In some embodiments, the test can include a further statistical test and a cut-off such as a P value below 0.1, 0.05, or 0.01, wherein a test polypeptide or nucleic acid encoding the same, needs to meet one or both thresholds (i.e., fold expansion and statistical cutoff).

For any of the lymphoproliferative element tests provided herein, the number of test cells and the number of control cells can be compared between day 7 and day 14, 21, 28, 35, 42 or 60 post-transduction. In some embodiments, the numbers of test and control cells can be determined by sequencing DNA and counting the occurrences of unique identifiers present in each construct. In some embodiments, the numbers of test and control cells can be counted directly, for example with a hemocytometer or a cell counter. In some embodiments, all the test cells and control cells can be grown within the same vessel, well or flask. In some embodiments, the test cells can be seeded in one or more wells, flasks or vessels, and the control cells can be seeded in one or more flasks or vessels. In some embodiments, test and control cells can be seeded individually into wells or flasks, e.g., one cell per well. In some embodiments, the numbers of test cells and control cells can be compared using enrichment levels. In some embodiments, the enrichment level for a test or control construct can be calculated by dividing the number of cells at a later time point (day 14, 21, 28, 35, or day 45) by the number of cells at day 7 for each construct. In some embodiments, the enrichment level for a test or control construct can be calculated by dividing the number of cells at a time point (day 14, 21, 28, 35, or day 45) by the number of cells at that time point for untransduced cells. In some embodiments, the enrichment level of each test construct can be normalized to the enrichment level of the respective control construct to generate a normalized enrichment level. In some embodiments, a LE encoded in the test construct provides (or a cell genetically modified and/or transduced with a retroviral particle (e.g. lentiviral particle) having a genome encoding the LE is capable of providing, is adapted for, possesses the property of, and/or is modified for) at least a 1.5-fold, 2-fold, 3-fold, 4-fold, 5-fold, 6-fold, 7-fold, 8-fold, 9-fold, or 10-fold normalized enrichment level, or between 1.5 fold and 25-fold normalized enrichment level, or between 3-fold and 20-fold normalized enrichment level, or between 5-fold and 25-fold normalized enrichment level, or between 5-fold and 20-fold normalized enrichment level, or between 5-fold and 15-fold normalized enrichment level. Enrichment can be measured, for example, by direct cell counting. Cutoff values can be based on a single test, or two, three, four, or five repeats, or based on many repeats. The cutoff can be met when a lymphoproliferative element meets one or more repeat tests, or meets or exceeds a cutoff for all repeats. In some embodiments, the enrichment is measured as log₂((normalized count data on the test day + 1)/(normalized count data on day 7 + 1)).

Additional details regarding the tests performed to identify the LEs are illustrated in WO2019/055946, including experimental conditions.

As illustrated in WO2019/055946, test constructs were identified as CLEs because the CLEs induced proliferation/expansion in these fed or unfed cultures without added cytokines such as IL-2 between days 7 and day 21, 28, 35, and/or 42. For example, as illustrated in WO2019/055946, effective CLEs were identified by identifying test CLEs that provided increased expansion of these in vitro cultures, whether fed or unfed with untransduced PBMCs, between day 7 and day 21, 28, 35, and/or 42 post-transduction, compared to control constructs that did not include any intracellular domains. WO2019/055946 discloses that at least one and typically more than one test CLE that included an intracellular domain from a test gene provided more expansion than every control construct that was present at day 7 post-transduction, that did not include an intracellular domain. WO2019/055946 further provides a statistical method that was used to identify exceptionally effective genes with respect to a first intracellular domain, and one or more exemplary intracellular domain(s) from these genes. The method used a Mann-Whitney-Wilcoxon test and a false discovery cutoff rate of less than 0.1 or less than 0.05. WO2019/055946 identified especially effective genes for the first intracellular domain or the second intracellular domain, for example, by analyzing scores for genes calculated as combined score for all constructs with that gene. Such analysis can use a cutoff of greater than 1, or greater than negative control constructs without any intracellular domains, or greater than 2, as shown for some of the tests disclosed in WO2019/055946.

In another embodiment, the LE provides, is capable of providing and/or possesses the property of (or a cell genetically modified and/or transduced with the LE is capable of providing, is adapted for, possesses the property of, and/or is modified for) driving T cell expansion in vivo. For example, the in vivo test can utilize a mouse model and measure T cell expansion at 15 to 25 days in vivo, or at 19 to 21 days in vivo, or at approximately 21 days in vivo, after T cells are contacted with lentiviral vectors encoding the LEs, are introduced into the mice, as disclosed in WO2019/055946,

In exemplary aspects and embodiments that include a LE, which typically include a CAR, such as methods provided herein for modifying, genetically modifying and/or transducing cells, and uses thereof, the genetically modified cell is modified so as to possess new properties not previously possessed by the cell before genetic modification and/or transduction. Such a property can be provided by genetic modification with a nucleic acid encoding a CAR or a LE, and in illustrative embodiments both a CAR and a LE. For example, in certain embodiments, the genetically modified and/or transduced cell is capable of, is adapted for, possesses the property of, and/or is modified for survival and/or proliferation in ex vivo culture for at least 7, 14, 21, 28, 35, 42, or 60 days or from between day 7 and day 14, 21, 28, 35, 42 or 60 post-transduction, in the absence of added IL-2 or in the absence of added cytokines such as IL-2, IL-15, or IL-7, and in certain illustrative embodiments, in the presence of the antigen recognized by the CAR where the method comprises modifying using a retroviral particle having a pseudotyping element and optionally a separate or fused activation domain on its surface and typically does not require pre-activation.

By capable of enhanced survival and/or proliferation in certain embodiments, it is meant that the genetically modified and/or transduced cell exhibits, is capable of, is adapted for, possesses the property of, and/or is modified for improved survival or expansion in ex vivo or in vitro culture in culture media in the absence of one or more added cytokines such as IL-2, IL-15, or IL-7, or added lymphocyte mitogenic agent, compared to a control cell(s) identical to the genetically modified and/or transduced cell(s) before it was genetically modified and/or transduced or to a control cell that was transduced with a retroviral particle identical to an on-test retroviral particle that comprises an LE or a putative LE, but without the LE or the intracellular domains of the LE, wherein said survival or proliferation of said control cell(s) is promoted by adding said one or more cytokines, such as IL-2, IL-15, or IL-7, or said lymphocyte mitogenic agent to the culture media. By added cytokine or lymphocyte mitogenic agent, it is meant that cytokine or lymphocyte mitogenic agent is added from an exogenous source to a culture media such that the concentration of said cytokine or lymphocyte mitogenic agent is increased in the culture media during culturing of the cell(s) compared to the initial culture media, and in some embodiments can be absent from the initial culture media before said adding. By “added” or “exogenously added”, it is meant that such cytokine or lymphocyte mitogenic agent is added to a lymphocyte media used to culture the modified, genetically modified, and/or transduced cell after the modifying, where the culture media may or may not already possess the cytokine or lymphocyte mitogenic agent. All or a portion of the media that includes a mixture of multiple media components is typically stored and in illustrative embodiments has been shipped to a site where the culturing takes place, without the exogenously added cytokine(s) or lymphocyte mitogenic agent(s). The lymphocyte media in some embodiments is purchased from a supplier, and a user such as a technician not employed by the supplier and not located within a supplier facility, adds the exogenously added cytokine or lymphocyte mitogenic agent to the lymphocyte media and then the genetically modified and/or transduced cells are cultured in the presence or absence of such exogenously added cytokine or lymphocyte mitogenic agent.

In some embodiments, improved or enhanced survival, expansion, and/or proliferation can be shown as an increase in the number of cells determined by sequencing DNA from cells transduced with retroviral particle (e.g. lentiviral particle) having a genome encoding CLEs and counting the occurrences of sequences present in unique identifiers from each CLE. In some embodiments, improved survival and/or improved expansion can be determined by counting the cells directly, for example with a hemocytometer or a cell counter, at each time point. In some embodiments, improved survival and/or improved expansion and/or enrichment can be calculated by dividing the number of cells at the later time point (day 21, 28, 35, and/or day 45) by the number of cells at day 7 for each construct. In some embodiments, the cells can be counted by hemocytometer or cell counters. In some embodiments, the enrichment level determined using the nucleic acid counts or the cell counts of each specific test construct can be normalized to the enrichment level of the respective control construct, i.e., the construct with the same extracellular domain and transmembrane domain but lacking the intracellular domains present in the test construct. In these embodiments, the LE encoded in the construct provides (or a cell genetically modified and/or transduced with a retroviral particle (e.g. lentiviral particle) having a genome encoding the LE is capable of providing, is adapted for, possesses the property of, and/or is modified for) at least a 1.5-fold, 2-fold, 3-fold, 4-fold, 5-fold, 6-fold, 7-fold, 8-fold, 9-fold, or 10-fold normalized enrichment level, or between 1.5 fold and 25-fold normalized enrichment level, or between 3-fold and 20-fold normalized enrichment level, or between 5-fold and 25-fold normalized enrichment level, or between 5-fold and 20-fold normalized enrichment level, or between 5-fold and 15-fold normalized enrichment level.

In some embodiments, the lymphoproliferative element can include a cytokine receptor or a fragment that includes a signaling domain thereof. In some embodiments, the cytokine receptor can be CD27, CD40, CRLF2, CSF2RA, CSF2RB, CSF3R, EPOR, GHR, IFNAR1, IFNAR2, IFNGR1, IFNGR2, IFNLR1, IL1R1, IL1RAP, IL1RL1, IL1RL2, IL2R, IL2RA, IL2RB, IL2RG, IL3RA, IL4R, IL5RA, IL6R, IL6ST, IL7R, IL7RA, IL9R, IL10RA, IL10RB, IL11RA, IL12RB1, IL13R, IL13RA1, IL13RA2, IL15R, IL15RA, IL17RA, IL17RB, IL17RC, IL17RE, IL18R1, IL18RAP, IL20RA, IL20RB, IL21R, IL22RA1, IL23R, IL27R, IL27RA, IL31RA, LEPR, LIFR, MPL, OSMR, PRLR, TGFβR, TGFβ decoy receptor, TNFRSF4, TNFRSF8, TNFRSF9, TNFRSF14, or TNFRSF18.

In some embodiments, a lymphoproliferative element, including a CLE, comprises an intracellular activating domain as disclosed hereinabove. In some illustrative embodiments a lymphoproliferative element is a CLE comprising an intracellular activating domain comprising an ITAM-containing domain, as such, the CLE can comprise an intracellular activating domain having at least 80%, 90%, 95%, 98%, or 100% sequence identity to the CD3Z, CD3D, CD3E, CD3G, CD79A, CD79B, DAP12, FCER1G, FCGR2A, FCGR2C, DAP10/CD28, or ZAP70 domains provided herein wherein the CLE does not comprise an ASTR.

In some embodiments, one or more domains of a lymphoproliferative element is fused to a modulatory domain, such as a co-stimulatory domain, and/or an intracellular activating domain of a CAR. In some embodiments of the composition and method aspects for transducing lymphocytes in whole blood, one or more intracellular domains of a lymphoproliferative element can be part of the same polypeptide as a CAR or can be fused and optionally functionally connected to some components of CARs. In still other embodiments, an engineered signaling polypeptide can include an ASTR, an intracellular activation domain (such as a CD3 zeta signaling domain), a co-stimulatory domain, and a lymphoproliferative domain. Further details regarding co-stimulatory domains, intracellular activating domains, ASTRs and other CAR domains, are disclosed elsewhere herein.

Lymphoproliferative elements provided herein typically include a transmembrane domain. For example, the transmembrane domain can have 80%, 85%, 90%, 95%, 97%, 98%, 99% or 100% sequence identity to any one of the transmembrane domains from the following genes and representative sequences disclosed in WO2019/055946: CD8 beta, CD4, CD3 zeta, CD28, CD134, CD7, CD2, CD3D, CD3E, CD3G, CD3Z, CD4, CD8A CD8B, CD27, CD28, CD40, CD79A, CD79B, CRLF2, CRLF2, CSF2RA, CSF2RB, CSF2RB, CSF3R, EPOR, FCER1G, FCGR2C, FCGRA2, GHR, ICOS, IFNAR, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL27RA, IL31RA, LEPR, LIFR, MPL, OSMR, PRLR, TNFRSF4, TNFRSF8, TNFRSF9, TNFRSF14, and TNFRSF18 or mutants thereof that are known to promote signaling activity in certain cell types if such mutants. Transmembrane (TM) domains suitable for use in any engineered signaling polypeptide include, but are not limited to, constitutively active cytokine receptors, the TM domain from LMP1, and TM domains from type 1 TM proteins comprising a dimerizing motif, as discussed in more detail herein. In any of the aspects disclosed herein containing the transmembrane domain from a type I transmembrane protein, the transmembrane domain can be a Type I growth factor receptor, a hormone receptor, a T cell receptor, or a TNF-family receptor.

In some embodiments, CLEs include both an extracellular portion and a transmembrane portion that is from the same protein, in illustrative embodiments the same receptor, either of which in illustrative embodiments is a mutant, thus forming an extracellular and transmembrane domain. These domains can be from a cytokine receptor, or a mutant thereof, or a hormone receptor, or a mutant thereof in some embodiments that have been reported to be constitutively active when expressed at least in some cell types. In illustrative embodiments, such extracellular and transmembrane domains do not include a ligand binding region. It is believed that such domains do not bind a ligand when present in CLEs and expressed in B cells, T cells, and/or NK cells. Mutations in such receptor mutants can occur in the transmembrane region or in the extracellular juxtamembrane region. Not to be limited by theory, a mutation in at least some extracellular — transmembrane domains of CLEs provided herein, are responsible for signaling of the CLE in the absence of ligand, by bringing activating chains together that are not normally together, or by changing the confirmation of a linked transmembrane and/or intracellular domain.

Exemplary extracellular and transmembrane domains for CLEs of embodiments that include such domains, in illustrative embodiments, are extracellular regions, typically less than 30 amino acids of the membrane-proximal extracellular domains along with transmembrane domains from mutant receptors that have been reported to be constitutive, that is not require ligand binding for activation of an associated intracellular domain. In illustrative embodiments, such extracellular and transmembrane domains include IL7RA Ins PPCL, CRLF2 F232C, CSF2RB V449E, CSF3R T640N, EPOR L251C I252C, GHR E260C I270C, IL27RA F523C, and MPL S505N. In some embodiments, the extracellular and transmembrane domain does not comprise more than 10, 20, 25 30 or 50 consecutive amino acids that are identical in sequence to a portion of the extracellular and/or transmembrane domain of IL7RA, or a mutant thereof. In some embodiments, the extracellular and transmembrane domain is other than IL7RA Ins PPCL. In some embodiments, the extracellular and transmembrane does not comprise more than 10, 20, 25, 30, or 50 consecutive amino acids that are identical in sequence to a portion of the extracellular and/or transmembrane domain of IL15R.

In embodiments of any of these aspects and embodiments wherein the transmembrane domain is a type I transmembrane protein, the transmembrane domain can be a Type I growth factor receptor, a hormone receptor, a T cell receptor, or a TNF-family receptor. In an embodiment of any of the aspects and embodiments wherein the chimeric polypeptide comprises an extracellular domain and wherein the extracellular domain comprises a dimerizing motif, the transmembrane domain can be a Type I cytokine receptor, a hormone receptor, a T cell receptor, or a TNF-family receptor.

In some embodiments, the extracellular and transmembrane domain is the viral protein LMP1, or a mutant and/or fragment thereof. LMP1 is a multispan transmembrane protein that is known to activate cell signaling independent of ligand when targeted to lipid rafts or when fused to CD40 (Kaykas et al. EMBO J. 20: 2641 (2001)). A fragment of LMP1 is typically long enough to span a plasma membrane and to activate a linked intracellular domain(s). For example, the LMP1 can be between 15 and 386, 15 and 200, 15 and 150, 15 and 100, 18 and 50, 18 and 30, 20 and 200, 20 and 150, 20 and 50, 20 and 30, 20 and 100, 20 and 40, or 20 and 25 amino acids. A mutant and/or fragment of LMP1 when included in a CLE provided herein, retains its ability to activate an intracellular domain. Furthermore, if present, the extracellular domain includes at least 1, but typically at least 4 amino acids and is typically linked to another functional polypeptide, such as a clearance domain, for example, an eTag. In some embodiments, the lymphoproliferative element comprises an LMP1 transmembrane domain. In illustrative embodiments, the lymphoproliferative element comprises an LMP1 transmembrane domain and the one or more intracellular domains do not comprise an intracellular domain from TNFRSF proteins (i.e. CD40, 4- IBB, RANK, TACI, OX40, CD27, GITR, LTR, and BAFFR), TLR1 to TLR13, integrins, FcyRIII, Dectinl, Dectin2, NOD1, NOD2, CD16, IL-2R, Type I II interferon receptor, chemokine receptors such as CCR5 and CCR7, G-protein coupled receptors, TREM1, CD79A, CD79B, Ig-alpha, IPS-1, MyD88, RIG-1, MDA5, CD3Z, MyD88ΔTIR, TRIF, TRAM, TIRAP, MAL, BTK, RTK, RAC1, SYK, NALP3 (NLRP3), NALP3ΔLRR, NALP1, CARD9, DAI, IPAG, STING, Zap70, or LAT.

In other embodiments of CLEs provided herein, the extracellular domain includes a dimerizing moiety. Many different dimerizing moieties disclosed herein can be used for these embodiments. In illustrative embodiments, the dimerizing moieties are capable of homodimerizing. Not to be limited by theory, dimerizing moieties can provide an activating function on intracellular domains connected thereto via transmembrane domains. In some embodiments, the dimerizing moiety of a CLE can be an anti-idiotype extracellular recognition domain of any of the anti-idiotype polypeptides herein. As such, anti-idiotype polypeptides containing an anti-idiotype extracellular domain can be CLEs. For example, the extracellular recognition domain attached to such a CLE can dimerize upon binding of the target antibody or antibody mimetic, as disclosed elsewhere herein. In other words, in some embodiments, the CLE is part of a fusion polypeptide including an anti-idiotype polypeptide, and the fusion polypeptide is dimerized through binding of the target antibody or antibody mimetic to the anti-idiotype extracellular recognition domain. In illustrative embodiments, the CLE is not constitutively active, but rather is activated upon dimerization induced by binding of a target antibody to 2 anti-idiotype polypeptides that bind the idiotype of the target antibody. In these embodiments, the target antibody typically does not induce cytotoxicity. In some embodiments, a lymphoproliferative element provided herein comprises an extracellular domain, and in illustrative embodiments, the extracellular domain comprises a dimerizing motif. In illustrative embodiments of this aspect, the extracellular domain comprises a leucine zipper. In some embodiments, the leucine zipper is from a jun polypeptide, for example c-jun. In certain embodiments the c-jun polypeptide is the c-jun polypeptide region of ECD-11.

An extracellular domain with a dimerizing moiety can also serve a function of connecting a cell tag polypeptide, such as an anti-idiotype extracellular recognition domain of an anti-idiotype polypeptide to a cell expressing a CLE. Accordingly, in such embodiments, the dimerizing motif can serve the function of a stalk connecting an anti-idiotype extracellular recognition domain to a membrane association domain, which in LEs and CLEs is typically a transmembrane domain. Such embodiments provide an advantage of having a transmembrane domain and dimerization motif that anchor an anti-idiotype domain to a cell, while retaining their function in an LE. In some embodiments, such embodiments provide the advantage of providing for tetramerization of an intracellular domain by constitutive dimerization through an LE dimerization motif and inducible dimerization, which would form tetramers, upon binding of a target antibody that has the idiotype recognized by the anti-idiotype extracellular recognition domain. In these embodiments, the target antibody typically does not induce cytotoxicity but rather serves to tetramerize dimerized ICDs. These are useful in some apoptosis-inducing embodiments as described in other sections herein.

In some embodiments, the dimerizing agent can be located intracellularly rather than extracellularly. In some embodiments, more than one or multiples of dimerizing domains can be used. In any aspects or embodiments wherein the extracellular domain of a CLE comprises a dimerizing motif, the dimerizing motif can be selected from the group consisting of: a leucine zipper motif-containing polypeptide, CD69, CD71, CD72, CD96, Cd105, Cd161, Cd162, Cd249, CD271, and Cd324, as well as mutants and/or active fragments thereof that retain the ability to dimerize. In any of the aspects and embodiments herein wherein the extracellular domain of a CLE comprises a dimerizing motif, the dimerizing motif can require a dimerizing agent, and the dimerizing motif and associated dimerizing agent can be selected from the group consisting of: FKBP and rapamycin or analogs thereof, GyrB and coumermycin or analogs thereof, DHFR and methotrexate or analogs thereof, or DmrB and AP20187 or analogs thereof, as well as mutants and/or active fragments of the recited dimerizing proteins that retain the ability to dimerize. In some aspects and illustrative embodiments, a lymphoproliferative element is constitutively active, and is other than a lymphoproliferative element that requires a dimerizing agent for activation.

Internally dimerizing and/or multimerizing lymphoproliferative elements in one embodiment are an integral part of a system that uses a dimeric analog of the lipid permeable immunosuppressant drug, FK506, which loses its normal bioactivity while gaining the ability to crosslink molecules genetically fused to the FK506-binding protein, FKBP12. By fusing one or more FKBPs and a myristoylation sequence to the cytoplasmic signaling domain of a target receptor, one can stimulate signaling in a dimerizer drug-dependent, but ligand and ectodomain-independent manner. This provides the system with temporal control, reversibility using monomeric drug analogs, and enhanced specificity. The high affinity of third- generation AP20187/AP1903 dimerizer drugs for their binding domain, FKBP12 permits specific activation of the recombinant receptor in vivo without the induction of non-specific side effects through endogenous FKBP12. FKBP12 variants having amino acid substitutions and deletions, such as FKBP12V36, that bind to a dimerizer drug, may also be used. In addition, the synthetic ligands are resistant to protease degradation, making them more efficient at activating receptors in vivo than most delivered protein agents.

Extracellular domains for embodiments where extracellular domains have a dimerizing motif, are long enough to form dimers, such as leucine zipper dimers. As such, extracellular domains that include a dimerizing moiety can be from 15 to 100, 20 to 50, 30 to 45, or 35 to 40 amino acids, of in illustrative embodiments is a c-Jun portion of a c-Jun extracellular domain. Extracellular domains of polypeptides that include a dimerizing moiety, may not retain other functionalities. For example, for leucine zippers embodiments, such leucine zippers are capable of forming dimers because they retain a motif of leucines spaced 7 residues apart along an alpha helix. However, leucine zipper moieties of certain embodiments of CLEs provided herein, may or may not retain their DNA binding function.

A spacer of between 1 and 4 alanine residues can be included in CLEs between the extracellular domain that has a dimerizing moiety, and the transmembrane domain. Not to be limited by theory, it is believed that the alanine spacer affects signaling of intracellular domains connected to the leucine zipper extracellular region via the transmembrane domain, by changing the orientation of the intracellular domains.

In illustrative embodiments, CLEs include a cell tag domain. Details regarding cell tags are provided in other sections herein. Any of the cell tags provided herein can be part of a CLE. Typically, the cell tag is linked to the N terminus of the extracellular domain. Not to be limited by theory, in some embodiments, the extracellular domain includes the function of providing a linker, in illustrative embodiments a flexible linker, linking a cell tag domain to a cell that expresses the CLE.

Furthermore, polynucleotides that include a nucleic acid sequence encoding a CLE provided herein, also typically comprise a signal sequence to direct expression to the plasma membrane. Exemplary signal sequences are provided herein in other sections. Elements can be provided on the transcript such that both a CAR and CLE are expressed from the same transcript in certain embodiments.

### Binding and Fusogenic Elements

Many of the methods, compositions, and kits provided herein include RIPs with envelope proteins on their surface, for example, multiple copies of a T cell and/or NK cell binding polypeptide and multiple copies of a fusogenic polypeptide, also called a fusogen. A “binding polypeptide” includes one or more polypeptides, typically glycoproteins, that identify and bind the target host cell. A “fusogenic polypeptide” mediates fusion of the retroviral and target host cell membranes, thereby allowing a retroviral genome to enter the target host cell. In certain embodiments, the binding polypeptide(s) and the fusogenic polypeptide(s) are on the same envelope protein, e.g., a heterologous glycoprotein. In other embodiments, the binding polypeptide(s) and the fusogenic polypeptide(s) are on two or more different heterologous glycoproteins.

One or both of these binding and fusogenic polypeptide functions can be provided by a pseudotyping element. In some embodiments, the pseudotyping element can be one or more viral envelope proteins. In some embodiments, the binding polypeptide function of a viral envelope protein can be altered, reduced, or eliminated (e.g., the amino acids corresponding to the binding polypeptide function can be mutated or deleted). In some embodiments, the viral envelope protein with reduced or eliminated binding polypeptide function can be retargeted with a new binding polypeptide function or by mutating the original binding polypeptide function.

In some embodiments, the binding polypeptide function can be provided by any polypeptide that binds to a cell surface marker on the target cell. For example, the binding polypeptide function can be provided by an activation element, as disclosed elsewhere herein. The pseudotyping of replication incompetent recombinant retroviral particles with heterologous envelope glycoproteins typically alters the tropism of a virus and facilitates the transduction of host cells. In some embodiments provided herein, pseudotyping elements are provided as polypeptide(s)/protein(s), or as nucleic acid sequences encoding the polypeptide(s)/protein(s).

In some embodiments, the pseudotyping element comprises the envelope protein from a different virus. In some embodiments, the pseudotyping element is the feline endogenous virus (RD114) envelope protein, an oncoretroviral amphotropic envelope protein, an oncoretroviral ecotropic envelope protein, the vesicular stomatitis virus envelope protein (VSV-G) (SEQ ID NO: 336), the baboon retroviral envelope glycoprotein (BaEV) (SEQ ID NO: 337), the murine leukemia envelope protein (MuLV) (SEQ ID NO: 338), the influenza glycoprotein HA surface glycoprotein (HA), the influenza glycoprotein neurominidase (NA), the paramyxovirus Measles envelope protein H, the paramyxovirus Measles envelope protein F, the Tupaia paramyxovirus (TPMV) envelope protein H, the TPMV envelope protein F, glycoproteins G and F from the Henipavirus genus, the Nipah virus (NiV) envelope protein F, the NiV envelope protein G, the Sindbis virus (SINV) protein E1, the SINV protein E2, and/or functional variants or fragments of any of these envelope proteins (see, e.g. Frank and Bucholz Mol Ther Methods Clin Dev. 2018 Oct 17;12:19-31).

In some embodiments, the pseudotyping element can be wild-type BaEV. Not to be limited by theory, BaEV contains an R peptide that has been shown to inhibit transduction. In some embodiments, the BaEV can contain a deletion of the R peptide. In some embodiments, the BaEV can contain a deletion of the inhibitory R peptide after the nucleotides encoding the amino acid sequence HA, referred to herein as BaEVΔR (HA) (SEQ ID NO: 339). In some embodiments, the BaEV can contain a deletion of the inhibitory R peptide after the nucleotides encoding the amino acid sequence HAM, referred to herein as BaEVΔR (HAM) (SEQ ID NO: 340).

In some embodiments, the pseudotyping element can be wild-type MuLV. In some embodiments, the MuLV can contain one or more mutations to remove the furin-mediated cleavage site located between the transmembrane (TM) and surface (SU) subunits of the envelope glycoprotein. In some embodiments the MuLV contains the SUx mutation (MuLVSUx) (SEQ ID NO:372) which inhibits furin-mediated cleavage of MuLV envelope protein in packaging cells. In certain embodiments the C-terminus of the cytoplasmic tail of the MuLV or MuLVSUx protein is truncated by 4 to 31 amino acids. In certain embodiments the C-terminus of the cytoplasmic tail of the MuLV or MuLVSUx protein is truncated by 4, 8, 12, 16, 20, 24, 28, or 31 amino acids.

In some embodiments, the pseudotyping elements include a binding polypeptide and a fusogenic polypeptide derived from different proteins. In one aspect, the pseudotyping element can comprise an influenza protein hemagglutinin HA and/or a neuraminidase (NA). In certain embodiments the HA is from influenza A virus subtype H1N1. In illustrative embodiments the HA is from H1N1 PR8 1934 in which the monobasic trypsin-dependent cleavage site has been mutated to a more promiscuous multibasic sequence (SEQ ID NO:311). In certain embodiments the NA is from influenza A virus subtype H10N7. In illustrative embodiments the NA is from H10N7-HKWF446C-07 (SEQ ID NO:312). In some embodiments, the binding polypeptide can be a functional variant or fragment of VSV-G, BaEV, BaEVΔR (HA), BaEVΔR (HAM), MuLV, MuLVSUx, influenza HA, influenza NA, or Measles envelope protein H that retains the ability to bind to a target cell, and the fusogenic polypeptide can be a functional variant or fragment of VSV-G, BaEV, BaEVΔR (HA), BaEVΔR (HAM), MuLV, MuLVSUx, influenza HA, influenza NA, or Measles envelope protein F that retains the ability to mediate fusion of the retroviral and target host cell membranes.

In another aspect, the replication incompetent recombinant retroviral particles of the methods and compositions disclosed herein can be pseudotyped with the fusion (F) and/or hemagglutinin (H) polypeptides of the measles virus (MV), as non-limiting examples, clinical wildtype strains of MV, and vaccine strains including the Edmonston strain (MV-Edm) (GenBank; AF266288.2) or fragments thereof. Not to be limited by theory, both hemagglutinin (H) and fusion (F) polypeptides are believed to play a role in entry into host cells wherein the H protein binds MV to receptors CD46, SLAM, and Nectin-4 on target cells and F mediates fusion of the retroviral and host cell membranes. In an illustrative embodiment, especially where the target cell is a T cell and/or NK cell, the binding polypeptide is a Measles Virus H polypeptide and the fusogenic polypeptide is a Measles Virus F polypeptide.

In some studies, lentiviral particles pseudotyped with truncated F and H polypeptides had a significant increase in titers and transduction efficiency (Funke et al. 2008. Molecular Therapy. 16(8):1427-1436), (Frecha et al. 2008. Blood. 112(13):4843-4852). The highest titers were obtained when the F cytoplasmic tail was truncated by 30 residues (referred to as MV(Ed)-FΔ30 (SEQ ID NO:313)). For the H variants, optimal truncation occurred when 18 or 19 residues were deleted (MV(Ed)-HΔ18 (SEQ ID NO:314) or MV(Ed)-HΔ19), although variants with a truncation of 24 residues with and without replacement of deleted residues with alanine (MV(Ed)-HΔ24 (SEQ ID NO:315) and MV(Ed)-HΔ24+A) also resulted in optimal titers. Accordingly, in some embodiments, including those directed to transducing T cells and/or NK cells, the replication incompetent recombinant retroviral particles of the methods and compositions disclosed herein are pseudotyped with mutated or variant versions of the measles virus fusion (F) and hemagglutinin (H) polypeptides, in illustrative examples, cytoplasmic domain deletion variants of measles virus F and H polypeptides. In some embodiments, the mutated F and H polypeptides are “truncated H” or “truncated F” polypeptides, whose cytoplasmic portion has been truncated, i.e. amino acid residues (or coding nucleic acids of the corresponding nucleic acid molecule encoding the protein) have been deleted. “HΔY” and “FΔX” designate such truncated H and F polypeptide, respectively, wherein “Y” refers to 1-34 residues that have been deleted from the amino termini and “X” refers to 1-35 residues that have been deleted from the carboxy termini of the cytoplasmic domains. In a further embodiment, the “truncated F polypeptide” is FΔ24 or FΔ30 and/or the “truncated H protein” is selected from the group consisting of HA14, HΔ15, HA16, HA17, HΔ18, HA19, HΔ20, HΔ21+A, HΔ24 and HΔ24+4A, more preferably HΔ18 or HΔ24. In an illustrative embodiment, the truncated F polypeptide is MV(Ed)-FΔ30 and the truncated H polypeptide is MV(Ed)-HΔ18.

In some embodiments, the pseudotyping elements can be the envelope proteins from the Henipavirus genus (e.g. Nipah, Hendra, Cedar, Mojiang or Kumasi virus) and include envelope glycoprotein G (Henipavirus-G protein) and their fusion partner envelope glycoprotein F (Henipavirus-F protein). In some embodiments, the Henipavirus-F protein comprises the sequence of SEQ ID NO:374 and the Henipavirus-G protein comprises the sequence of SEQ ID NO:375. In some embodiments, the Henipavirus-F protein comprises a sequence that has at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:374. In some embodiments, the Henipavirus-G protein comprises a sequence that has at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:375.

In some embodiments, the Henipavirus-G protein can contain one or more mutations to modify (e.g., truncate) the cytoplasmic tail and thus improve pseudotyping and particle incorporation efficiency(Palomares et al. 2013. J Virol. 87(8):4794-4794; Witting et al. 2013. Gene Ther. 20(10):997-1005; Bender et al. 2016. Plos Pathog. 12(6): e1005641). In certain embodiments, the N-terminus of the cytoplasmic tail of any of the Henipavirus-G proteins can be truncated by 1 to all of its amino acids. In some embodiments, the residues of the Henipavirus-G protein involved in receptor binding are mutated to alter, and in illustrative embodiments remove, their native interactions with their natural receptors. In certain embodiments, the Henipavirus-G protein is mutated for example, but not limited to, at one or more of Y389, E501, W504, E505, V507, Q530, E533, or I588 of SEQ ID NO:375 (amino acids are given for Nipah-G, also referred to as NiV-G, and a skilled artisan will be able to identify the corresponding glutamines of other Henipavirus-G proteins)(Guillaume et al. 2006. J Virol. 80(15):7546-7554; Negrete et al. 2007. J Virol. 81(19):10804-10814; Xu et al. 2008. P Natl Acad Sci USA. 105(29):9953-9958; Xu et al. 2012. Plos One. 7(11): e48742; Bender et al. 2016. Plos Pathog. 12(6): e1005641). In some embodiments, Henipavirus-G protein is SEQ ID NO:375 with mutations E533A and/or Q530A. In some embodiments, one or more N- or O-glycosylation sites are mutated to improve pseudotyping and fusion (Biering et al. 2012. J Virol. 86(22):11991-12002; Stone et al. 2016. Plos Pathog. 12(2): e1005445). In some embodiments, one or more N-glycosylation sites are mutated for example, but not limited to, at one or more of N72, N159, N306, N378, N417, N481, or N529 of SEQ ID NO:375, or the corresponding glutamines of other Henipavirus-G proteins, to another amino acid such as glutamine. In some embodiments, one or more O-glycosylation sites are mutated from serine or threonine to another amino acid such as alanine. In some embodiments, one or more of the serine or threonine residues in the heavily O-glycosylated stalk domain from amino acids 103 to 137 of SEQ ID NO:375, is mutated to, for example, alanine. In other embodiments, the C-terminus of the Henipavirus-G protein can be modified and fused to a binding polypeptide and in illustrative embodiments, an activation element, such as an antibody or antibody mimetic, which in illustrative embodiments can be an anti-CD3 antibody or antibody mimetic (Bender et al. 2016. Plos Pathog. 12(6): e1005641; Jamali et al. 2019. Mol Ther-Meth Clin D. 13:371-379; Frank et al. 2020. Blood Adv. 4(22):5702-5715).

In some embodiments, the F proteins can contain one or more mutations to modify (e.g., truncate) the cytoplasmic tail and thus improve pseudotyping, particle incorporation efficiency, and/or cleavage of the F protein from the inactive F0 to the cleaved active F1 form (Khetawat et al. 2010. Virol J. 7:312; Palomares et al. 2013. J Virol. 87(8):4794-4794; Witting et al. 2013. Gene Ther. 20(10):997-1005; Bender et al. 2016. Plos Pathog. 12(6): e1005641; Johnston et al. 2017. J Virol. 91(10): e02150-16). In some embodiments, one or more N-glycosylation sites are mutated for example, but not limited to, at one or more of N64, N67, N99, N414, or N 464 to another amino acid such as glutamine. In certain embodiments, the C-terminus of the cytoplasmic tail of the envelope glycoprotein F from the Henipavirus genus (Henipavirus-F protein) is truncated by 1 to all of its amino acids. In some embodiments, the F protein can contain one or more mutations to make it more fusogenic (Aguilar et al. 2007. J Virol. 81(9):4520-4532; Weis et al. 2015. Eur J Cell Biol. 94(7-9):316-322).

In some embodiments, the pseudotyping element can include a Henipavirus-F protein and a Henipavirus-G protein from the same virus of the Henipavirus genus (i.e., homologous proteins). In some embodiments, the pseudotyping element can include a Henipavirus-F protein and a Henipavirus-G protein from different viruses of the Henipavirus genus (i.e., heterologous proteins). In some embodiments, the pseudotyping element can include a Henipavirus-F protein and a Henipavirus-G protein can be chimeras composed of domains of heterologous proteins (Bradel-Tretheway et al. 2019. J Virol. 93(13): e00577-19).

In some embodiments, any of the pseudotyping elements can contain one or more mutations to modify (e.g., truncate) the cytoplasmic tail and thus improve pseudotyping, and particle incorporation efficiency. In certain embodiments, the N-terminus of the cytoplasmic tail is truncated by 1 to all of its amino acids. In some embodiments, the residues involved in receptor binding are mutated to alter, and in illustrative embodiments remove, their native interactions with their natural receptors. Similar to the mutations for Nipah-G protein, in some embodiments, the VSV-G protein is mutated for example, but not limited to, in the residues K47 or R354, for example K47A or K47Q and/or R354A or R354Q. In some embodiments, these pseudotyping elements are fused to heterologous binding polypeptides that function to direct or redirect the pseudotyping element to a new target protein on the same or different cell target.

In some embodiments, the separate binding and/or fusogenic polypeptides comprise one or more non virally-derived proteins. In some embodiments the binding polypeptide comprises an antibody, a ligand, or a receptor that binds a polypeptide on the target cell. In some embodiments, the binding polypeptide comprises an alternative non-antibody scaffold, also referred to herein as an antibody mimetic. In any of the aspects or embodiments provided herein that include a binding polypeptide, the binding polypeptide can be an antibody mimetic. In any of the aspects or embodiments provided herein that include a binding polypeptide that is an antibody, a suitable antibody mimetic can be used instead of the antibody. In some embodiments, the antibody mimetic can be an affibody, an afflilin, an affimer, an affitin, an alphabody, an alphamab, an anticalin, a peptide aptamer, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a nanofitin, a leucine-rich repeat domain, a lipocalin domain, a mAb 2 or Fcab™, a nanobody, a nanoCLAMP, an OBody, a Pronectin, a single-chain TCR, a tetratricopeptide repeat domain, VHH, or a V-like domain. In some embodiments the binding polypeptide recognizes a protein on the surface of NK cells such as CD16, CD56, and CD57. In some embodiments the binding polypeptide recognizes a protein on the surface of T cells such as CD3, CD4, CD8, CD25, CD28, CD62L, CCR7, TCRa, and TCRb. In some embodiments, the binding polypeptide is also the activation element. In some embodiments, the binding polypeptide is a membrane polypeptide that binds CD3. In some embodiments, the fusogen is derived from the Sindbis virus glycoprotein that is modified to remove its binding activity, SV1, and the binding polypeptide is a membrane-bound anti-CD3 antibody (Yang et al. 2009. Pharm Res 26(6):1432-1445).

In some embodiments, the viral particles are copseudotyped with envelope glycoproteins from 2 or more heterologous viruses. In some embodiments, the viral particles are copseudotyped with VSV-G, or a functional variant or fragment thereof, and an envelope protein from RD114, BaEV, MuLV, influenza virus, measles virus, and/or a functional variant or fragment thereof. In some embodiments, the viral particles are copseudotyped with VSV-G and the MV(Ed)-H glycoprotein or the MV(Ed)-H glycoprotein with a truncated cytoplasmic domain. In illustrative embodiments, the viral particles are copseudotyped with VSV-G and MV(Ed)-HΔ24. In certain embodiments, VSV-G is copseudotyped with MuLV or MuLV with a truncated cytoplasmic domain. In other embodiments, VSV-G is copseudotyped with MuLVSUx or MuLVSUx with a truncated cytoplasmic domain. In further illustrative embodiments, VSV-G is copseudotyped with a fusion of an antiCD3scFv to MuLV.

In some embodiments, the fusogenic polypeptide is derived from a class I fusogen. In some embodiments, the fusogenic polypeptide is derived from a class II fusogen. In some embodiments, both the binding polypeptide and the separate fusogenic polypeptide are virally-derived. In some embodiments, the fusogenic polypeptide includes multiple elements expressed as one polypeptide. In some embodiments, the binding polypeptide and fusogenic polypeptide are translated from the same transcript but from separate ribosome binding sites; in other embodiments, the binding polypeptide and fusogenic polypeptide are separated by a cleavage peptide site, which not to be bound by theory, is cleaved after translation, as is common in the literature, or a ribosomal skip sequence. In some embodiments, the translation of the binding polypeptide and fusogenic polypeptide from separate ribosome binding sites results in a higher amount of the fusogenic polypeptide as compared to the binding polypeptide. In some embodiments, the ratio of the fusogenic polypeptide to the binding polypeptide is at least 2:1, at least 3:1, at least 4:1, at least 5:1, at least 6:1, at least 7:1, or at least 8:1. In some embodiments, the ratio of the fusogenic polypeptide to the binding polypeptide is between 1.5:1, 2:1, or 3:1, on the low end of the range, and 3:1, 4:1, 5:1, 6:1, 7:1, 8:1. 9:1 or 10:1 on the high end of the range.

In embodiments disclosed herein including short contacting times, many of the modified lymphocytes in a cell formulation have pseudotyping elements on their surfaces during reintroduction of the modified lymphocytes into the subject, either through association with the replication incompetent recombinant retroviral particle or by fusion of the retroviral envelopes with the plasma membranes of the modified lymphocytes. In some embodiments, at least 10%, 15%, 20%, 25%, 30%, 35%, 40%, 45%, 50%, 55%, 60%, 65%, 70%, 75%, 80%, 85%, or 90% of the modified lymphocytes in the cell formulation can include a pseudotyping element on their surfaces. In some embodiments, the pseudotyping element can be bound to the surface of the modified lymphocytes and/or the pseudotyping element can be present in the plasma membrane of the modified lymphocytes.

In some embodiments, RIPs herein include on their surface, a means for binding to a T cell and/or NK cell. In some embodiments, RIPs herein include on their surface, a means for mediating fusion of the RIP and T cell and/or NK cell membranes. In some embodiments, RIPs herein include on their surface, both a means for binding to a T cell and/or NK cell, and a means for mediating fusion of the RIP and T cell and/or NK cell membranes. In some embodiments, this is the same means.

### Activation Elements

Many of the methods and composition aspects of the present disclosure that include a replication incompetent recombinant retroviral particle further include an activation element, also referred to herein as a T cell activation element, or a nucleic acid encoding an activation element. Activation elements are envelope proteins of the replication incompetent recombinant retroviral particles. Cells of the immune system such as T lymphocytes recognize and interact with specific antigens through receptors or receptor complexes which, upon recognition or an interaction with such antigens, cause activation of the cell and expansion in the body. An example of such a receptor is the antigen-specific T lymphocyte receptor complex (TCR/CD3) expressed on the surface of T lymphocytes. The TCR recognizes antigenic peptides that are presented to it by the proteins of the major histocompatibility complex (MHC) on the surface of antigen presenting cells and other T lymphocyte targets. Stimulation of the TCR/CD3 complex results in activation of the T lymphocyte and a consequent antigen-specific immune response. Thus, activation elements provided herein, activate T cells by binding to one or more components of the T cell receptor associated complex, for example by binding to CD3. In some embodiments, the activation element can activate alone. In other cases, the activation requires activation through the TCR receptor complex in order to further activate cells. T lymphocytes also require a second, co-stimulatory signal to become fully active in vivo. Without such a signal, T lymphocytes are either non-responsive to antigen binding to the TCR, or become anergic. However, the second, co-stimulatory signal is not required for the transduction and expansion of T cells and can be provided, for example, by a later co-stimulatory signal from a CAR or LE after transduction, as provided elsewhere herein. In some embodiments, the co-stimulatory signal can be provided during transduction by, for example, CD28, a T lymphocyte protein, which interacts with CD80 and CD86 on antigen-producing cells.

Activation of the T cell receptor (TCR) CD3 complex and co-stimulation with CD28 can occur by ex vivo exposure to solid surfaces (e.g., beads) coated with anti-CD3 and anti-CD28. In some embodiments of the methods and compositions disclosed herein, resting T cells are activated by exposure to solid surfaces coated with anti-CD3 and anti-CD28 ex vivo. In other embodiments, resting T cells or NK cells, and in illustrative embodiments resting T cells, are activated by exposure to soluble anti-CD3 antibodies (e.g., at 50-150, or 75-125, or 100 ng/ml). In such embodiments, which can be part of methods for modifying, genetically modifying or transducing, in illustrative embodiments without prior activation, such activation and/or contacting can be carried out by including anti-CD3 in a transduction reaction mixture and contacting with optional incubating for any of the times provided herein. Furthermore, such activation with soluble anti-CD3 can occur by incubating lymphocytes, such as PBMCs, and in illustrative embodiments NK cells and in more illustrative embodiments, T cells, after they are contacted with retroviral particles in a media containing an anti-CD3. Such incubation can be for example, for between 5, 10, 15, 30, 45, 60, or 120 minutes on the low end of the range, and 15, 30, 45, 60, 120, 180, or 240 minutes on the high end of the range, for example, between 15 and 1 hours or 2 hours.

In certain illustrative embodiments of the methods, kits, and compositions provided herein, for example for modifying, genetically modifying, and/or transducing lymphocytes, especially T cells and/or NK cells, polypeptides that are capable of binding to an activating T cell surface protein are presented as “activation elements” on the surface of replication incompetent recombinant retroviral particles. Thus, in some embodiments, an activation element can perform the binding polypeptide function. In some embodiments, the activation element is an envelope protein. Such T cell and/or NK cell activation elements on the surface of a retroviral particle are present in embodiments herein for modifying, genetically modifying, and/or transducing lymphocytes, for example wherein the retroviral particle has a genome that encodes a CAR, self-driving CAR, or LE. In some embodiments, such retroviral particles whose surface has an activation element are used in methods and uses that include administration by subcutaneous administration, and in kit components for subcutaneous administration. The activation element function discussed herein this section, as well as the binding polypeptide and fusogenic polypeptide disclosed elsewhere herein, in certain illustrative embodiments are all found associated with the surface of a retroviral particle, as part of one, two, or three proteins, in illustrative embodiments glycoproteins, and in further illustrative embodiments, heterologous glycoproteins. For example, some activation element polypeptides, such as those that are capable of binding to CD3, can also provide a T cell binding polypeptide function.

In some embodiments, the activation element is a polypeptide capable of binding to a polypeptide on the surface of a lymphocyte, and in illustrative embodiments, a T cell and/or an NK cell. In illustrative embodiments, the activation element is capable of binding to a TCR complex polypeptide. In some embodiments, a TCR complex polypeptide is CD3D, CD3E, CD3G, CD3Z, TCRα, or TCRβ. In some embodiments, the activation element capable of binding to the TCR complex polypeptide is a polypeptide capable of binding to one or more of CD3D, CD3E, CD3G, CD3Z, TCRα, or TCRβ. In illustrative embodiments, the activation element activates ZAP-70. In some embodiments, the activation element includes a polypeptide capable of binding to CD16A, NKG2C, NKG2D, NKG2E, NKG2F, or NKG2H. In some embodiments, the polypeptide capable of binding to NKG2D is MIC-A, MIC-B, or a ULBP, for example ULBP1 or ULBP2. In further embodiments, the polypeptide capable of binding to CD16A includes capable of binding to one or more of NKp46, 2B4, CD2, DNAM, NKG2C, NKG2D, NKG2E, NKG2F, or NKG2H. In some embodiments, the activation element is a polypeptide capable of binding to one or more of the following combinations: NKp46 and 2B4, NKp46 and CD2, NKp46 and DNAM, NKp46 and NKG2D, 2B4 and DNAM, or 2B4 and NKG2D. In some embodiments, the activation element can be two or more polypeptides capable of binding to polypeptides on the surface of a lymphocyte. In some embodiments, the activation element can be one or more polypeptides capable of binding to at least one of the following combinations: NKp46 and 2B4, NKp46 and CD2, NKp46 and DNAM, NKp46 and NKG2D, 2B4 and DNAM, or 2B4 and NKG2D. In illustrative embodiments the activation element is a polypeptide capable of binding to CD3E. In some embodiments, the polypeptide capable of binding to CD3 is an anti-CD3 antibody, or a fragment thereof that retains the ability to bind to CD3. In illustrative embodiments, the anti-CD3 antibody or fragment thereof is a single chain anti-CD3 antibody, such as but not limited to, an anti-CD3 scFv. In another illustrative embodiment, the polypeptide capable of binding to CD3 is anti-CD3scFvFc. In some embodiments, the activation element is an antibody. In some embodiments, the activation element comprises an alternative non-antibody scaffold, also referred to herein as an antibody mimetic. In any of the aspects or embodiments provided herein that include an activation element capable of binding to a polypeptide on the surface of a lymphocyte, and in illustrative embodiments, a T cell, the binding polypeptide can be an antibody mimetic. In some embodiments, the antibody mimetic can be an affibody, an afflilin, an affimer, an affitin, an alphabody, an alphamab, an anticalin, a peptide aptamer, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a nanofitin, a leucine-rich repeat domain, a lipocalin domain, a mAb 2 or Fcab™, a nanobody, a nanoCLAMP, an OBody, a Pronectin, a single-chain TCR, a tetratricopeptide repeat domain, VHH, or a V-like domain. In any of the aspects or embodiments provided herein that include an activation element that is an antibody, a suitable antibody mimetic can be used instead of the antibody. In some embodiments, the activation element capable of binding a polypeptide on the surface of a lymphocyte (e.g. TCRβ) is a superantigen polypeptide.

A number of anti-human CD3 monoclonal antibodies and antibody fragments thereof are available, and can be used as T cell activation elements in the present invention, including but not limited to UCHT1, OKT-3, HIT3A, TRX4, X35-3, VIT3, BMA030 (BW264/56), CLB-T3/3, CRIS7, YTH12.5, F111409, CLB-T3.4.2, TR-66, TR66.opt, HuM291, WT31, WT32, SPv-T3b, 11D8, XIII-141, XIII46, XIII-87, 12F6, T3/RW2-8C8, T3/RW24B6, OKT3D, M-T301, SMC2 and F101.01. As such, RIPs herein include on their surface, a means for activating T cells.

In other embodiments, the activation element on the surfaces of the replication incompetent recombinant retroviral particles can include one or more polypeptides capable of binding CD2, CD28, OX40, 4-1BB, ICOS, CD9, CD53, CD63, CD81, and/or CD82 and optionally one or more polypeptides capable of binding CD3. In illustrative embodiments, the activation element is a polypeptide capable of binding a mitogenic tetraspanin, for example, a polypeptide capable of binding to CD81, CD9, CD53, CD63, or CD82. In some embodiments, the activation element is a tetraspanin. Tetraspanins are known in the art. In some embodiments, the tetraspanin can be TSPAN1 (TSP-1), TSPAN2 (TSP-2), TSPAN3 (TSP-3), TSPAN4 (TSP-4, NAG-2), TSPAN5 (TSP-5), TSPAN6 (TSP-6), TSPAN7 (CD231/TALLA-⅟A15), TSPAN8 (CO-029), TSPAN9 (NET-5), TSPAN10 (OCULOSPANIN), TSPAN11 (CD151-like), TSPAN12 (NET-2), TSPAN13 (NET-6), TSPAN14, TSPAN15 (NET-7), TSPAN16 (TM4-B), TSPAN17, TSPAN18, TSPAN19, TSPAN20 (UP1b, UPK1B), TSPAN21 (UP1la, UPK1A), TSPAN22 (RDS, PRPH2), TSPAN23 (ROM1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31 (SAS), TSPAN32 (TSSC6), or TSPAN33. In some embodiments, the tetraspanin can be TSPAN1 (TSP-1), TSPAN2 (TSP-2), TSPAN3 (TSP-3), TSPAN4 (TSP-4, NAG-2), TSPAN5 (TSP-5), TSPAN6 (TSP-6), TSPAN7 (CD231/TALLA-⅟A15), TSPAN8 (CO-029), TSPAN9 (NET-5), TSPAN10 (OCULOSPANIN), TSPAN11 (CD151-like), TSPAN12 (NET-2), TSPAN13 (NET-6), TSPAN14, TSPAN15 (NET-7), TSPAN16 (TM4-B), TSPAN17, TSPAN18, TSPAN19, TSPAN20 (UP1b, UPK1B), TSPAN21 (UP1a, UPK1A), TSPAN22 (RDS, PRPH2), TSPAN23 (ROM1), TSPAN24 (CD151), TSPAN26 (CD37), TSPAN31 (SAS), TSPAN32 (TSSC6), or TSPAN33. In illustrative embodiments, the tetraspanin is TSPAN7 (CD231/TALLA-⅟A15), TSPAN9 (NET-5), TSPAN24 (CD151), TSPAN27 (CD82), TSPAN28 (CD81), TSPAN29 (CD9), or TSPAN30 (CD63). In some embodiments, the activation element is a tetraspanin, and the tetraspanin is TSPAN25 (CD53), TSPAN27 (CD82), TSPAN28 (CD81), TSPAN29 (CD9), or TSPAN30 (CD63). In some embodiments, a tetraspanin is the only envelope protein. In some embodiments, a tetraspanin is a pseudotyping element comprising the binding polypeptide and the fusogenic element. In some embodiments, a tetraspanin is the activation element and the pseudotyping element. In illustrative embodiments, the tetraspanin that is the activation element and the pseudotyping element is TSPAN29 (CD9).

In some embodiments, one or typically more copies of one or more of these activation elements can be expressed on the surfaces of the replication incompetent recombinant retroviral particles as polypeptides separate and distinct from the pseudotyping elements. In some embodiments, the activation elements can be expressed on the surfaces of the replication incompetent recombinant retroviral particles as fusion polypeptides. In illustrative embodiments, the fusion polypeptides include one or more activation elements and one or more pseudotyping elements or one or more binding and/or fusogenic elements. In further illustrative embodiments, the fusion polypeptide includes anti-CD3, for example an anti-CD3scFv, or an anti-CD3scFvFc, and a viral envelope protein. In one example the fusion polypeptide is the OKT-3scFv fused to the amino terminal end of a viral envelope protein such as the MuLV envelope protein, as shown in Maurice et al. (2002). In some embodiments, the fusion polypeptide is UCHT1scFv fused to a viral envelope protein, for example the MuLV envelop protein (SEQ ID NO:341), the MuLVSUx envelope protein (SEQ ID NO:366), VSV-G (SEQ ID NO:367), or functional variants or fragments thereof, including any of the membrane protein truncations provided herein. In illustrative embodiments, especially for compositions and methods herein for transducing lymphocytes in whole blood, the fusion polypeptide does not include any blood protein (e.g., blood Factor (e.g., Factor X)) cleavage sites in the portion of the fusion protein that resides outside the retroviral particle. In some embodiments, the fusion constructs do not include any furin cleavage sites. Furin is a membrane bound protease expressed in all mammalian cells examined, some of which is secreted and active in blood plasma (See e.g., C. Fernandez et al. J. Internal. Medicine (2018) 284; 377-387). Mutations can be made to fusion constructs using known methods to remove such protease cleavage sites.

Polypeptides that bind CD3, CD28, OX40, 4-1BB, or ICOS are referred to as activation elements because of their ability to activate resting T cells. In certain embodiments, nucleic acids encoding such an activation element are found in the genome of a replication incompetent recombinant retroviral particle that contains the activating element on its surface. In illustrative embodiments, nucleic acids encoding an activation element are not found in the replication incompetent recombinant retroviral particle genome. In some embodiments, the nucleic acids encoding an activation element are found in the genome of a virus packaging cell.

In some embodiments, the activation element is a polypeptide capable of binding to CD28, for example, an anti-CD28 antibody or an anti-CD28 scFv antibody, or a fragment thereof that retains the ability to bind to CD28. In other embodiments, the polypeptide capable of binding to CD28 is CD80, CD86, or a functional fragment thereof that is capable of binding CD28 and inducing CD28-mediated activation of Akt, such as an external fragment of CD80. In some aspects herein, an external fragment of CD80 means a fragment that is typically present on the outside of a cell in the normal cellular location of CD80, that retains the ability to bind to CD28.

Anti-CD28 antibodies are known in the art and can include, as non-limiting examples, the monoclonal antibodies 9.3 (an IgG2a antibody), KOLT-2 (an IgG1 antibody), 15E8 (an IgG1 antibody), 248.23.2 (an IgM antibody), and EX5.3D10 (an IgG2a antibody).

In an illustrative embodiment, an activation element includes two polypeptides, a polypeptide capable of binding to CD3 and a polypeptide capable of binding to CD28.

In certain embodiments, the polypeptide capable of binding to CD3 or CD28 is an antibody, a single chain monoclonal antibody or an antibody fragment, for example a single chain antibody fragment. Accordingly, the antibody fragment can be, for example, a single chain fragment variable region (scFv), an antibody binding (Fab) fragment of an antibody, a single chain antigen-binding fragment (scFab), a single chain antigen-binding fragment without cysteines (scFabΔC), a fragment variable region (Fv), a construct specific to adjacent epitopes of an antigen (CRAb), or a single domain antibody (VH or VL).

In some embodiments, at least 10%, 15%, 20%, 25%, 30%, 35%, 40%, 45%, 50%, 55%, 60%, 65%, 70%, 75%, 80%, 85%, or 90% of the modified lymphocytes in a cell formulation can include a T cell activation element on their surfaces. In some embodiments, the T cell activation element can be bound to the surface of the modified lymphocytes through, for example, a T cell receptor, and/or the pseudotyping element can be present in the plasma membrane of the modified lymphocytes.

In any of the embodiments disclosed herein, an activation element, or a nucleic acid encoding the same, can include a dimerizing or higher order multimerizing motif. Dimerizing and multimerizing motifs are well-known in the art and a skilled artisan will understand how to incorporate them into the polypeptides for effective dimerization or multimerization. In illustrative embodiments, the polypeptide capable of binding to CD3 is anti-CD3scFvFc, which in some embodiments is considered an anti-CD3 with a dimerizing motif without any additional dimerizing motif, since anti-CD3scFvFc constructs are known to be capable of dimerizing without the need for a separate dimerizing motif.

In some embodiments, when present on the surface of replication incompetent recombinant retroviral particles, an activation element including a dimerizing motif can be active in the absence of a dimerizing agent. In some embodiments, the dimerizing or multimerizing motif, or a nucleic acid sequence encoding the same, can be an amino acid sequence from transmembrane polypeptides that naturally exist as homodimers or multimers. In some embodiments, the dimerizing or multimerizing motif, or a nucleic acid sequence encoding the same, can be an amino acid sequence from a fragment of a natural protein or an engineered protein. In one embodiment, the homodimeric polypeptide is a leucine zipper motif-containing polypeptide (leucine zipper polypeptide). For example, a leucine zipper polypeptide derived from c-JUN, non-limiting examples of which are disclosed related to chimeric lymphoproliferative elements (CLEs) herein. In some embodiments, these transmembrane homodimeric polypeptides can include CD69, CD71, CD72, CD96, Cd105, Cd161, Cd162, Cd249, CD271, Cd324, or active fragments thereof.

In some embodiments, when present on the surface of replication incompetent recombinant retroviral particles, an activation element including a dimerizing motif can be active in the presence of a dimerizing agent. In some embodiments, the dimerizing motif, and nucleic acid encoding the same, can include an amino acid sequence from transmembrane proteins that dimerize upon ligand (also referred to herein as a dimerizer or dimerizing agent) binding. In some embodiments, the dimerizing motif and dimerizer can include (where the dimerizer is in parentheses following the dimerizer-binding pair): FKBP and FKBP (rapamycin or its analog); GyrB and GyrB (coumermycin or its analog); DHFR and DHFR (methotrexate); or DmrB and DmrB (AP20187). As noted above, rapamycin can serve as a dimerizer. Alternatively, a rapamycin derivative or analog can be used (see, e.g., WO96/41865; WO 99/36553; WO 01/14387; and Ye et al (1999) Science 283:88-91). A coumermycin analog can be used (see, e.g., Farrar et al. (1996) Nature 383:178-181; and U.S. Pat. No. 6,916,846). Although some embodiments of lymphoproliferative elements include a dimerizing agent, in some aspects and illustrative embodiments, a lymphoproliferative element is constitutively active, and is other than a lymphoproliferative element that requires a dimerizing agent for activation.

In some embodiments, an activation element is fused to a heterologous signal sequence and/or a heterologous membrane attachment sequence or a membrane bound protein, all of which help direct the activation element to the membrane. In some embodiments, posttranslational lipid modification can occur via myristoylation, palmitoylation, or GPI anchorage. In some embodiments, the heterologous membrane attachment sequence is a GPI anchor attachment sequence. The heterologous GPI anchor attachment sequence can be derived from any known GPI-anchored protein. In some embodiments, the heterologous GPI anchor attachment sequence is the GPI anchor attachment sequence from CD14, CD16, CD48, CD55 (DAF), CD59, CD80, and CD87. In some embodiments, the heterologous GPI anchor attachment sequence is derived from CD16. In illustrative embodiments, the heterologous GPI anchor attachment sequence is derived from Fc receptor FcγRIIIb (CD16b) or decay accelerating factor (DAF), otherwise known as complement decay-accelerating factor or CD55. In some embodiments, the activation element is attached to the membrane via an endogenous transmembrane protein.

In some embodiments, one or more of the activation elements include a heterologous signal sequence to help direct expression of the activation element to the cell membrane. Any signal sequence that is active in the packaging cell line can be used. In some embodiments, the signal sequence is a DAF signal sequence. In illustrative embodiments, an activation element is fused to a DAF signal sequence at its N terminus and a GPI anchor attachment sequence at its C terminus.

In an illustrative embodiment, the activation element includes anti-CD3 scFvFc fused to a GPI anchor attachment sequence derived from CD14 and CD80 fused to a GPI anchor attachment sequence derived from CD16b; and both are expressed on the surface of a replication incompetent recombinant retroviral particle provided herein. In some embodiments, the anti-CD3 scFvFc is fused to a DAF signal sequence at its N terminus and a GPI anchor attachment sequence derived from CD14 at its C terminus and the CD80 is fused to a DAF signal sequence at its N terminus and a GPI anchor attachment sequence derived from CD16b at its C terminus; and both are expressed on the surface of a replication incompetent recombinant retroviral particle provided herein. In some embodiments, the DAF signal sequence includes amino acid residues 1-30 of the DAF protein.

In some embodiments, an activation element can be separate from the replication incompetent recombinant retroviral particle. Thus, in some embodiments, the replication incompetent recombinant retroviral particle do not comprise an activation element on their surface.

Nucleic acid aptamers can serve as alternative molecules to antibodies for binding and/or eliciting biologic function, including for use as immunotherapeutics (Freage 2020). In any of the aspects or embodiments provided herein that include an activation element including an activation element on the surface of a RIP, a suitable DNA aptamer can be used instead of the polypeptide. Therefore, in certain embodiments, a nucleic acid aptamer capable of binding CD2, CD28, OX40, 4-1BB, ICOS, CD9, CD53, CD63, CD81, and/or CD82 is used in place of a ploypeptide. In some embodiments, a DNA aptamer capable of binding CD3 is used in place of a polypeptide capable of binding CD3. In some embodiments, ZUCH-1 or variants thereof, is used to bind CD3 and activate T-cells.

In some embodiments, more than one activation element is used. In some embodiments, the activation element can be a superantigen, for example lipopolysaccharide, SEC3, and Staphylococcal enterotoxin B. In some embodiments, the activation element can be a cytokine. In some embodiments, the activation element can be phorbol myristate acetate (PMA), ionomycin, or phytohemagglutinin (PHA). In some embodiments, the concentration of PMA in a cell formulation or to be administered separately from the replication incompetent recombinant retroviral particles can be 10, 25, 50, 75, or 100 ng/ml or between 10 and 100 ng/ml or 25 and 75 ng/ml. In some embodiments, the concentration of ionomycin in a cell formulation or to be administered separately from the replication incompetent recombinant retroviral particles can be at least or about 100, 250, 500, or 750 ng/ml or 1, 2, 3, 4, or 5 µg/ml or between 100 ng/ml and 5 µg/ml or between 500 ng/ml and 2 µg/ml. In some embodiments, the concentration of PHA in a cell formulation or to be administered separately from the replication incompetent recombinant retroviral particles can be at least or about 0.1 µg/ml, 0.25 µg/ml, 0.5 µg/ml, 1 µg/ml, 2.5 µg/ml, 5 µg/ml, 7.5 µg/ml, or 10 µg/ml or between 0.1 and 10 µg/ml, 1 and 10 µg/ml, or 2.5 and 7.5 µg/ml. In some embodiments, the activation element is administered within 5, 10, 15, 20, 30, 45, or 60 minutes, or 1, 2, 3, 4, 5, 6, 7, 8, 10, 12, 18, or 24 hours or 1, 2, 3, 4, 5, 6, 7, 14, 21, or 28 days of administering a cell formulation. In some embodiments, the activation element or elements are administered multiple times, for example on different days following administration of the cell formulation.

### Membrane-Bound Cytokines

Some embodiments of the method and composition aspects provided herein, include a membrane-bound cytokine, or polynucleotides encoding a membrane-bound cytokine. Cytokines are typically, but not always, secreted proteins. Cytokines that are naturally secreted can be engineered as fusion proteins to be membrane-bound. Membrane-bound cytokine fusion polypeptides are included in methods and compositions disclosed herein, and are also an aspect of the invention. In some embodiments, replication incompetent recombinant retroviral particles have a membrane-bound cytokine fusion polypeptide on their surface that is capable of binding a T cell and/or NK cell and promoting proliferation and/or survival thereof. Typically, membrane-bound polypeptides are incorporated into the membranes of replication incompetent recombinant retroviral particles, and when a cell is transduced by the replication incompetent recombinant retroviral particles, the fusion of the retroviral and host cell membranes results in the polypeptide being bound to the membrane of the transduced cell.

In some embodiments, the cytokine fusion polypeptide includes one or more of IL-2, IL-7, IL-15, or an active fragment.

The membrane-bound cytokine fusion polypeptides are typically a cytokine fused to heterologous signal sequence and/or a heterologous membrane attachment sequence. In some embodiments, the heterologous membrane attachment sequence is a GPI anchor attachment sequence. The heterologous GPI anchor attachment sequence can be derived from any known GPI-anchored protein (reviewed in Ferguson MAJ, Kinoshita T, Hart GW. Glycosylphosphatidylinositol Anchors. In: Varki A, Cummings RD, Esko JD, et al., editors. Essentials of Glycobiology. 2nd edition. Cold Spring Harbor (NY): Cold Spring Harbor Laboratory Press; 2009. Chapter 11). In some embodiments, the heterologous GPI anchor attachment sequence is the GPI anchor attachment sequence from CD14, CD16, CD48, CD55 (DAF), CD59, CD80, and CD87. In some embodiments, the heterologous GPI anchor attachment sequence is derived from CD16. In an illustrative embodiment, the heterologous GPI anchor attachment sequence is derived from Fc receptor FcγRIIIb (CD16b). In some embodiments, the GPI anchor is the GPI anchor of DAF.

In illustrative embodiments, the membrane-bound cytokine is a fusion polypeptide of a cytokine fused to DAF. DAF is known to accumulate in lipid rafts that are incorporated into the membranes of replication incompetent recombinant retroviral particles budding from packaging cells. Accordingly, not to be limited by theory, it is believed that DAF fusion proteins are preferentially targeted to portions of membranes of packaging cells that will become part of a recombinant retroviral membrane.

In non-limiting illustrative embodiments, the cytokine fusion polypeptide is an IL-7, or an active fragment thereof, fused to DAF. In a specific non-limiting illustrative embodiment, the fusion cytokine polypeptide includes in order: the DAF signal sequence (residues 1-31 of DAF), IL-7 without its signal sequence, and residues 36-525 of DAF.

In some embodiments, a membrane-bound cytokine fusion polypeptide comprises a cleavage site. In some embodiments, the cleavage site can be within the sequence of the cytokine. In some embodiments, the cleavage site can be within the sequence of the heterologous signal sequence. In some embodiments, the cleavage site can be within the sequence of the heterologous membrane attachment sequence. In some embodiments, the cleavage site can be between the cytokine and the heterologous signal sequence or the heterologous membrane attachment sequence.

In some embodiments, the membrane-bound cytokine fusion polypeptide can include linkers as disclosed elsewhere herein.

### Packaging Cell Lines/Methods of Making Recombinant Retroviral Particles

The present disclosure provides mammalian packaging cells and packaging cell lines that produce replication incompetent recombinant retroviral particles. The cell lines that produce replication incompetent recombinant retroviral particles are also referred to herein as packaging cell lines. A non-limiting example of such method is illustrated in WO2019/055946. Further exemplary methods for making retroviral particles are provided herein, for example in the Examples section herein. Such methods include, for example, a 4 plasmid system or a 5 plasmid system when a nucleic acid encoding an additional membrane bound protein, such as a T cell activation element that is not a fusion with the viral envelope, such as a GPI-linked anti-CD3, is included (See WO2019/05546). In an illustrative embodiment, provided herein is a 4 plasmid system in which a T cell activation element, such as a GPI-linked anti-CD3, is encoded on one of the packaging plasmids such as the plasmid encoding the viral envelope or the plasmid encoding REV, and optionally a second viral membrane-associated transgene such as a membrane bound cytokine can be encoded on the other packaging plasmid. In each case the nucleic acid encoding the viral protein is separated from the transgene by an IRES or a ribosomal skip sequence such as P2A or T2A. Such 4 plasmid system and associated polynucleotides as stated in the Examples, provided increased titers as compared to a 5 vector system in transient transfections, and thus provide illustrative embodiments herein. The present disclosure provides packaging cells and mammalian cell lines that are packaging cell lines that produce replication incompetent recombinant retroviral particles that genetically modify target mammalian cells and the target mammalian cells themselves. In illustrative embodiments, the packaging cell comprises nucleic acid sequences encoding a packageable RNA genome of the replication incompetent retroviral particle, a REV protein, a gag polypeptide, a pol polypeptide, and a pseudotyping element.

The cells of the packaging cell line can be adherent or suspension cells. Exemplary cell types are provided hereinbelow. In illustrative embodiments, the packaging cell line can be a suspension cell line, i.e., a cell line that does not adhere to a surface during growth. The cells can be grown in a chemically-defined media and/or a serum-free media. In some embodiments, the packaging cell line can be a suspension cell line derived from an adherent cell line, for example, the HEK293 cell line can be grown in conditions to generate a suspension-adapted HEK293 cell line according to methods known in the art. The packaging cell line is typically grown in a chemically defined media. In some embodiments, the packaging cell line media can include serum. In some embodiments, the packaging cell line media can include a serum replacement, as known in the art. In illustrative embodiments, the packaging cell line media can be serum-free media. Such media can be a chemically defined, serum-free formulation manufactured in compliance with Current Good Manufacturing Practice (CGMP) regulations of the US Food and Drug Administration (FDA). The packaging cell line media can be xeno-free and complete. In some embodiments, the packaging cell line media has been cleared by regulatory agencies for use in ex vivo cell processing, such as an FDA 510(k) cleared device.

Accordingly, in one aspect, provided herein is a method of making a replication incompetent recombinant retroviral particle including: A. culturing a packaging cell in suspension in serum-free media, wherein the packaging cell comprises nucleic acid sequences encoding a packageable RNA genome of the replication incompetent retroviral particle, a REV protein, a gag polypeptide, a pol polypeptide, and a pseudotyping element; and B. harvesting the replication incompetent recombinant retroviral particle from the serum-free media. In another aspect, provided herein is a method of transducing a lymphocyte with a replication incompetent recombinant retroviral particle comprising: A. culturing a packaging cell in suspension in serum-free media, wherein the packaging cell comprises nucleic acid sequences encoding a packageable RNA genome of the replication incompetent retroviral particle, a REV protein, a gag polypeptide, a pol polypeptide, and a pseudotyping element; B. harvesting the replication incompetent recombinant retroviral particle from the serum-free media; and C. contacting the lymphocyte with the replication incompetent recombinant retroviral particle, wherein the contacting is performed for less than 24 hours, 20 hours, 18 hours, 12 hours, 8 hours, 4 hours, 2 hours, 1 hour, 30 minutes, or 15 minutes (or between contacting and no incubation, or 15 minutes, 30 minutes, 1, 2, 3, or 4 hours on the low end of the range and 1, 2, 3, 4, 6, 8, 12, 18, 20, or 24 hours on the high end of the range), thereby transducing the lymphocyte.

The packageable RNA genome, in certain illustrative embodiments, is designed to express one or more target polypeptides, including as a non-limiting example, any of the engineered signaling polypeptides disclosed herein and/or one or more (e.g., two or more) inhibitory RNA molecules in opposite orientation (e.g., encoding on the opposite strand and in the opposite orientation), from retroviral components such as gag and pol. For example, the packageable RNA genome can include from 5′ to 3′: a 5′ long terminal repeat, or active truncated fragment thereof; a nucleic acid sequence encoding a retroviral cis-acting RNA packaging element; a nucleic acid sequence encoding a first and optionally second target polypeptide, such as, but not limited to, an engineered signaling polypeptide(s) in opposite orientation, which can be driven off a promoter in this opposite orientation with respect to the 5′ long terminal repeat and the cis-acting RNA packaging element, which in some embodiments is called a “fourth” promoter for convenience only (and sometimes referred to herein as the promoter active in T cells and/or NK cells), which is active in a target cell such as a T cell and/or an NK cell but in illustrative examples is not active in the packaging cell or is only inducibly or minimally active in the packaging cell; and a 3′ long terminal repeat, or active truncated fragment thereof. In some embodiments, the packageable RNA genome can include a central polypurine tract (cPPT)/central termination sequence (CTS) element. In some embodiments, the retroviral cis-acting RNA packaging element can be HIV Psi. In some embodiments, the retroviral cis-acting RNA packaging element can be the Rev Response Element. The engineered signaling polypeptide driven by the promoter in the opposite orientation from the 5′ long terminal repeat, in illustrative embodiments, is one or more of the engineered signaling polypeptides disclosed herein and can optionally express one or more inhibitory RNA molecules as disclosed in more detail herein and in WO2017/165245A2, WO2018/009923A1, and WO2018/161064A1. In some aspects, provided herein is a packageable RNA genome designed to express a self-driving CAR. Details regarding such replication incompetent recombinant retroviral particles, and composition and method aspects including a self-driving CAR, are disclosed in more detail herein, for example in the Self-Driving CAR Methods and Compositions section and in the Exemplary Embodiments section. In illustrative embodiments, the first one or more transcriptional units encoding a lymphoproliferative element is encoded in the reverse orientation and the second one or more transcriptional units encoding a CAR is in the forward orientation.

It will be understood that promoter number, such as a first, second, third, fourth, etc. promoter is for convenience only. A promoter that is called a “fourth” promoter should not be taken to imply that there are any additional promoters, such as first, second or third promoters, unless such other promoters are explicitly recited. It should be noted that each of the promoters are capable of driving expression of a transcript in an appropriate cell type and such transcript forms a transcription unit.

In some embodiments, the engineered signaling polypeptide can include a first lymphoproliferative element. Suitable lymphoproliferative elements are disclosed in other sections herein. As a non-limiting example, the lymphoproliferative element can be expressed as a fusion with a cell tag, such as an eTag, as disclosed herein. In some embodiments, the packageable RNA genome can further include a nucleic acid sequence encoding a second engineered polypeptide including a chimeric antigen receptor, encoding any CAR embodiment provided herein. For example, the second engineered polypeptide can include a first antigen-specific targeting region, a first transmembrane domain, and a first intracellular activating domain. Examples of antigen-specific targeting regions, transmembrane domains, and intracellular activating domains are disclosed elsewhere herein. In some embodiments where the target cell is a T cell, the promoter that is active in a target cell is active in a T cell, as disclosed elsewhere herein.

In some embodiments, the engineered signaling polypeptide can include a CAR, and the nucleic acid sequence can encode any CAR embodiment provided herein. For example, the engineered polypeptide can include a first antigen-specific targeting region, a first transmembrane domain, and a first intracellular activating domain. Examples of antigen-specific targeting regions, transmembrane domains, and intracellular activating domains are disclosed elsewhere herein. In some embodiments, the packageable RNA genome can further include a nucleic acid sequence encoding a second engineered polypeptide. In some embodiments, the second engineered polypeptide can be a lymphoproliferative element. In some embodiments where the target cell is a T cell or NK cell, the promoter that is active in a target cell is active in a T cell or NK cell, as disclosed elsewhere herein.

In some embodiments, the packageable RNA genome included in any of the aspects provided herein, can further include a riboswitch, as discussed in WO2017/165245A2, WO2018/009923A1, and WO2018/161064A1. In some embodiments, the nucleic acid sequence encoding the engineered signaling polypeptide can be in a reverse orientation with respect to the 5′ to 3′ orientation established by the 5′ LTR and the 3′ LTR. In further embodiments, the packageable RNA genome can further include a riboswitch and, optionally, the riboswitch can be in reverse orientation. In any of the embodiments disclosed herein, a polynucleotide including any of the elements can include a primer binding site. In illustrative embodiments, insulators and/or polyadenylation sequences can be placed before, after, between, or near genes to prevent or reduce unregulated transcription. In some embodiments, the insulator can be chicken HS4 insulator, Kaiso insulator, SAR/MAR elements, chimeric chicken insulator-SAR elements, CTCF insulator, the gypsy insulator, or the β-globin insulator or fragments thereof known in the art. In some embodiments, the insulator and/or polyadenylation sequence can be hGH polyA (SEQ ID NO:316), SPA1 (SEQ ID NO:317), SPA2 (SEQ ID NO:318), b-globin polyA spacer B (SEQ ID NO:319), b-globin polyA spacer A (SEQ ID NO:320), 250 cHS4 insulator v1 (SEQ ID NO:321), 250 cHS4 insulator v2 (SEQ ID NO:322), 650 cHS4 insulator (SEQ ID NO:323), 400 cHS4 insulator (SEQ ID NO:324), 650 cHS4 insulator and b-globin polyA spacer B (SEQ ID NO:325), or b-globin polyA spacer B and 650 cHS4 insulator (SEQ ID NO:326).

In any of the embodiments disclosed herein, a nucleic acid sequence encoding Vpx can be on the second or an optional third transcriptional unit, or on an additional transcriptional unit that is operably linked to the first inducible promoter.

Some aspects of the present disclosure include or are cells, in illustrative examples, mammalian cells, that are used as packaging cells to make replication incompetent recombinant retroviral particles, such as lentiviruses, for transduction of T cells and/or NK cells. In some aspects, provided herein are packaging cells to make replication incompetent recombinant retroviral particles that include a polynucleotide encoding a self-driving CAR. Details regarding such replication incompetent recombinant retroviral particles, and composition and method aspects including a self-driving CAR, are disclosed in more detail herein, for example in the Self-Driving CAR Methods and Compositions section and in the Exemplary Embodiments section.

Any of a wide variety of cells can be selected for in vitro production of a virus or virus particle, such as a redirected recombinant retroviral particle, according to the invention. Eukaryotic cells are typically used, particularly mammalian cells including human, simian, canine, feline, equine and rodent cells. In illustrative examples, the cells are human cells. In further illustrative embodiments, the cells reproduce indefinitely, and are therefore immortal. Examples of cells that can be advantageously used in the present invention include NIH 3T3 cells, COS cells, Madin-Darby canine kidney cells, human embryonic 293T cells and any cells derived from such cells, such as gpnlslacZ φNX cells, which are derived from 293T cells. Highly transfectable cells, such as human embryonic kidney 293T cells, can be used. By “highly transfectable” it is meant that at least about 50%, more preferably at least about 70% and most preferably at least about 80% of the cells can express the genes of the introduced DNA.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL1O), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, Hut-78, Jurkat, HL-60, and the like.

### Nucleic Acids

The present disclosure provides polynucleotides with nucleic acids encoding polypeptides of the present disclosure and nucleic acids are disclosed for use in various methods herein. A nucleic acid will in some embodiments be DNA, including, e.g., a recombinant expression construct, or as all or part of the genome of a T cell or an NK cell, for example. A nucleic acid will in some embodiments be RNA, such as a retroviral genome or an expressed transcript within a packaging cell line, a T cell or an NK, for example. A nucleic acid will in some embodiments be RNA, e.g., in vitro synthesized RNA. In some embodiments, the nucleic acid can be isolated. As used herein, the term “isolated” means that the material is removed from its original environment (e.g., the natural environment if it is naturally occurring). For example, a naturally-occurring polynucleotide, or in other embodiments a polypeptide, present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. For example, an isolated nucleic can be part of recombinant nucleic acid vector, such as an expression vector, which in illustrative embodiments can be a replication incompetent recombinant retroviral particle. In some embodiments, the nucleic acid is manufactured in compliance with cGMP, as discussed herein for kit components.

In some embodiments, a nucleic acid provides for production of a polypeptide of the present disclosure, e.g., in a mammalian cell. In other cases, a subject nucleic acid provides for amplification of the nucleic acid encoding a polypeptide of the present disclosure.

Some polynucleotides provided herein, include promoters. Promoters suitable for use may be constitutive or inducible. For expression of the viral particle RNA, an LTR promoter or hybrid LTR promoter may be used. For example, RSV/LTR, TRE/LTR or LTR alone may be used to transcribe the nucleic acid to be packaged. Examples of LTR’s include, but are not limited to, MSCV, GALV, HIV-1, HIV-2 and MuLV. In packaging lines containing the large T antigen, for example, incorporation of the SV40 origin of replication may be included in one or more packaging vectors to amplify circular plasmid DNA during transcription and/or translation. The use of multiple promoters may be utilized to prevent transcription factor competition. For example CMV, SV40, RSV, HSVTK, TRE and other promoters may be utilized to express different components of the LV particles. In some instances, viral particle components may be expressed from integrated expression vectors. In other cases, one or more of the nucleic acids may be introduced via transient expression. In some embodiments the use of inducible promoters are used to minimize cellular toxicity before viral particle packaging.

For expression of a transgene (e.g. a CAR or an anti-idiotype polypeptide) in a genetically modified cell, such as a lymphocyte, a macrophage, or a dendritic cell, suitable promoters include any constitutive promoter known in the art. In some embodiments, the constitutive promoter can be an EF1-a promoter, PGK promoter, CMV promoter, MSCV-U3 promoter (see, e.g., Jones et al., Human Gene Therapy (2009) 20: 630-40), SV40hCD43 promoter, VAV promoter, TCRβ promoter, UBC promoter, cytomegalovirus immediate early promoter, herpes simplex virus thymidine kinase promoter, early and late SV40 promoters, promoter present in long terminal repeats from a retrovirus, mouse metallothionein-I promoter, and various art-known tissue specific promoters. In some embodiments, a constitutive promoter can include the EF1-a promoter nucleotide sequence (SEQ ID NO:350), the PGK promoter nucleotide sequence (SEQ ID NO:351), or a functional portion or variant thereof. In some embodiments, a constitutive promoter can include other than the EF1-a promoter. In some embodiments, the promoter include light and/or heavy chain immunoglobulin gene promoter and enhancer elements.

In some embodiments, the promoter is not active in the packaging line or is only minimally active in the packaging line. Such embodiments have the advantage with expression an engineered T cell receptor or a CAR, that they would reduce, minimize, or in illustrative embodiments substantially eliminate, or even eliminate expression of the engineered T cell receptor or CAR in a encapsulated nucleic acid vector such as a RIR retroviral particle or a virus-like particle because of reduced, low, negligible, substantially no, or no expression of the engineered T cell receptor or CAR in the packaging cell line used to make the encapsulated nucleic acid vector. In illustrative embodiments, such expression is reduced, substantially eliminated, or eliminated on the surface of the encapsulated nucleic acid vector (e.g. RIR particle or virus-like particle). In some embodiments, the promoter can be a T cell-specific promoter, a CD8 cell-specific promoter, a CD4 cell-specific promoter, a NKT cell specific promoter, or an NK cell-specific promoter. In some embodiments, the T cell-specific promoter can be the CD3 zeta promoter or the CD3 delta promoter (see, e.g., Ji et al., J Biol Chem. 2002 Dec 6;277(49):47898-906). In illustrative embodiments, the T cell-specific promoter can be the CD3 zeta promoter. In some embodiments, the T cell-specific promoter can be a CD8 gene promoter. In some embodiments, the T cell-specific promoter can be a CD4 gene promoter (see, e.g., Salmon et al. (1993) Proc. Natl. Acad. Sci. USA 90:7739; and Marodon et al. (2003) Blood 101:3416). In some embodiments, an NK cell-specific promoter can be a Neri (p46) promoter (see, e.g., Eckelhart et al. (2011) Blood 117:1565). In some embodiments, the specific proteins encoded by the recombinant polynucleotide vector are not expressed, displayed, and/or incorporated in the surface of the polynucleotide vector (e.g. RIP). In some embodiments, this is accomplished by means of a T cell specific promoter driving transgene expression in the polynucleotide vector. In some embodiments, that promoter is a promoter from the CD3 family. In other embodiments it is a hybrid CD3 promoter. In other embodiments, the packaging cell line encodes a repressor protein capable of substantially suppressing the expression of the lentiviral transgenes in the packaging cell line. In some embodiments that suppressor may be a TET repressor protein. In yet other embodiments, the transcription factor activate against the protein in the packaging line has been suppressed or inactivated. In some embodiments the inactivation may be achieved through DNA editing nucleases. In other embodiments the inactivation is achieved through shRNA or miRNA. In other embodiments, the suppression of the transcription factor is achieved through a dominant negative protein or degron to the transcription factor. In other embodiments, the viral nucleic acids are controlled via a ligand inducible or repressible promoter not activated in the packaging cell line.

In other embodiments, the promoter can be a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like. Further discussion of suitable promoters for use in various methods and as separate aspects, are provided herein.

In some embodiments, the promoter is inducible in a cell to be genetically modified (e.g. CAR-T cell). In some embodiments, an inducible promoter can include a T cell-specific response element or an NFAT response element. In other embodiments the promoter may be regulated by an environmental condition, such as hypoxia, temperature, glucose, pH or light. In other embodiments the promoter may be responsive to concentrations of extracellular molecules. In some instances, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., PNAS (2000) 28: e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. (2006) Annual Review of Biochemistry, 567-605 and Tropp (2012) Molecular Biology (Jones & Bartlett Publishers, Sudbury, MA), the disclosures of which are incorporated herein by reference.

In some aspects, provided herein are polynucleotides that include a promoter that is particularly useful for a self-driving CAR. Details regarding such promoters, and composition and method aspects including a self-driving CAR that contain such promoters, are disclosed in more detail herein, for example in the Self-Driving CAR Methods and Compositions section and in the Exemplary Embodiments section. In some cases, the promoter is a CD8 cell-specific promoter, a CD4 cell-specific promoter, a macrophage-specific promoter, or an NK-specific promoter. For example, a CD4 gene promoter can be used.

An isolated nucleotide sequence encoding a polypeptide of the disclosure can be present in a eukaryotic expression vector and/or a cloning vector. Nucleotide sequences encoding two separate polypeptides can be cloned in the same or separate vectors. An expression vector can include a selectable marker, an origin of replication, and other features that provide for replication and/or maintenance of the vector and expression of a transgene. For example, an expression vector typically includes a promoter operably linked to a transgene. Suitable expression vectors are known in the art and include, for example, plasmids and viral vectors. In some embodiments, the expression vector is a recombinant retroviral particle, as disclosed in detail herein.

Expression vectors generally have convenient restriction sites located near the promoter sequence to provide for the insertion of nucleic acid sequences encoding heterologous proteins. A selectable marker operative in the expression host may be present.

As noted above, in some embodiments, a nucleic acid encoding a polypeptide of the present disclosure will in some embodiments be RNA, e.g., in vitro synthesized RNA. Methods for in vitro synthesis of RNA are known in the art; any known method can be used to synthesize RNA including a nucleotide sequence encoding a polypeptide of the present disclosure. Methods for introducing RNA into a host cell are known in the art. See, e.g., Zhao et al. (2010) Cancer Res. 15:9053. Introducing RNA including a nucleotide sequence encoding a polypeptide of the present disclosure into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding a polypeptide of the present disclosure.

Various aspects and embodiments that include a polynucleotide, a nucleic acid sequence, and/or a transcriptional unit, and/or a vector including the same, further include one or more of a Kozak-type sequence (also called a Kozak-related sequence herein), a woodchuck hepatitis virus post-transcriptional regulatory element (WPRE), and a double stop codon or a triple stop codon, wherein one or more stop codons of the double stop codon or the triple stop codon define a termination of a reading from of at least one of the one or more transcriptional units. In certain embodiments, a polynucleotide a nucleic acid sequence, and/or a transcriptional unit, and/or a vector including the same, further includes a Kozak-type sequence having a 5′ nucleotide within 10 nucleotides upstream of a start codon of at least one of the one or more transcriptional units. Kozak determined the Kozak consensus sequence, (GCC)GCCRCCATG (SEQ ID NO:327), for 699 vertebrate mRNAs, where R is a purine (A or G) (Kozak. Nucleic Acids Res. 1987 Oct 26;15(20):8125-48). In one embodiment the Kozak-type sequence is or includes CCACCAT/UG(G) (SEQ ID NO:328), CCGCCAT/UG(G) (SEQ ID NO:329), GCCGCCGCCAT/UG(G) (SEQ ID NO:330), or GCCGCCACCAT/UG(G) (SEQ ID NO:331) (with nucleotides in parenthesis representing optional nucleotides and nucleotides separated by a slash indicated different possible nucleotides at that position, for example depending on whether the nucleic acid is DNA or RNA. In these embodiments that include the AU/TG start codon, the A can be considered position 0. In certain illustrative embodiments, the nucleotides at -3 and +4 are identical, for example the -3 and +4 nucleotides can be G. In another embodiment the Kozak-type sequence includes an A or G in the 3rd position upstream of ATG where ATG is the start codon. In another embodiment the Kozak-type sequence includes an A or G in the 3rd position upstream of AUG where AUG is the start codon. In an illustrative embodiment, the Kozak sequence is (GCC)GCCRCCATG (SEQ ID NO:327), where R is a purine (A or G). In an illustrative embodiment, the Kozak-type sequence is GCCGCCACCAUG (SEQ ID NO:332). In another embodiment, which can be combined with the preceding embodiment that includes a Kozak-type sequence and/or the following embodiment that includes triple stop codon, the polynucleotide includes a WPRE element. WPREs have been characterized in the art (See e.g., (Higashimoto et al., Gene Ther. 2007; 14: 1298)) and as illustrated in WO2019/055946. In some embodiments, the WPRE element is located 3′ of a stop codon of the one or more transcriptional units and 5′ to a 3′ LTR of the polynucleotide. In another embodiment, which can be combined with either or both of the preceding embodiments (i.e. an embodiment wherein the polynucleotide includes a Kozak-type sequence and/or an embodiment wherein the polynucleotide includes a WPRE), the one or more transcriptional units terminates with one or more stop codons of a double stop codon or a triple stop codon, wherein the double stop codon includes a first stop codon in a first reading frame and a second stop codon in a second reading frame, or a first stop codon in frame with a second stop codon, and wherein the triple stop codon includes a first stop codon in a first reading frame, a second stop codon in a second reading frame, and a third stop codon in a third reading frame, or a first stop codon in frame with a second stop codon and a third stop codon.

A triple stop codon herein includes three stop codons, one in each reading frame, within 10 nucleotides of each other, and preferably having overlapping sequence, or three stop codons in the same reading frame, preferably at consecutive codons. A double stop codon means two stop codons, each in a different reading frame, within 10 nucleotides of each other, and preferably having overlapping sequences, or two stop codons in the same reading frame, preferably at consecutive codons.

In some of the methods and compositions disclosed herein, the introduction of DNA into PBMCs, B cells, T cells and/or NK cells and optionally the incorporation of the DNA into the host cell genome, is performed using methods that use recombinant nucleic acid vectors other than replication incompetent recombinant retroviral particles. For example, other viral vectors can be utilized, such as those derived from adenovirus, adeno-associated virus, or herpes simplex virus-1, as non-limiting examples.

In some embodiments, methods provided herein, and associated uses, reaction mixtures, kits and cell formulations can include transfecting cells with polynucleotides that are not encoded in viral vectors. Such polynucleotides can be referred to as non-viral vectors. In any of the embodiments disclosed herein that utilize non-viral vectors to genetically modify or transfect cells, the non-viral vectors, including for example, plasmids or naked DNA, can be introduced into the cells, such as for example, PBMCs, B cells, T cells and/or NK cells using methods that include electroporation, nucleofection, liposomal formulations, lipids, dendrimers, cationic polymers such as poly(ethylenimine) (PEI) and poly(1-lysine) (PLL), nanoparticles, cell-penetrating peptides, microinjection, and/or non-integrating lentiviral vectors. In some embodiments, the liposomal formulations, lipids, dendrimers, PEI, PLL, nanoparticles, and cell-penetrating peptides can be modified to include lymphocyte-targeting ligands, for example, an anti-CD3 antibody. PEI coupled to anti-CD3 antibodies was shown to efficiently transfect PBMCs with an exogenous nucleic acid (O′Neill et al. Gene Ther. 2001 Mar;8(5):362-8). Similarly, nanoparticles made from polyglutamic acid molecules coupled to anti-CD3e f(ab′)2 fragments transfected T lymphocytes (Smith et al. Nat Nanotechnol. 2017 Aug; 12(8): 813-820). In some embodiments, DNA can be introduced into cells, such as PBMCs, B cells, T cells and/or NK cells in a complex with liposomes and protamine. Other methods for transfecting T cells and/or NK cells ex vivo that can be used in embodiments of methods provided herein, are known in the art (see e.g., Morgan and Boyerinas, Biomedicines. 2016 Apr 20; 4(2). pii: E9, incorporated by reference herein in its entirety).

In some embodiments of method provided herein, DNA can be integrated into the genome using transposon-based carrier systems by co-transfection, co-nucleofection or co-electroporation of target DNA as plasmid containing the transposon ITR fragments in 5′ and 3′ ends of the gene of interest and transposase carrier system as DNA or mRNA or protein or site specific serine recombinases such as phiC31 that integrates the gene of interest in pseudo attP sites in the human genome, in this instance the DNA vector contains a 34 to 40 bp attB site that is the recognition sequence for the recombinase enzyme (Bhaskar Thyagarajan et al. Site-Specific Genomic Integration in Mammalian Cells Mediated by Phage <pC31 Integrase, Mol Cell Biol. 2001 Jun; 21(12): 3926-3934) and co transfected with the recombinase. For T cells and/or NK cells, transposon-based systems that can be used in certain methods provided herein utilize the Sleeping Beauty DNA carrier system (see e.g., U.S. Pat. No. 6,489,458 and U.S. Pat. Appl. No. 15/434,595, incorporated by reference herein in their entireties), the PiggyBac DNA carrier system (see e.g., Manuri et al., Hum Gene Ther. 2010 Apr;21(4):427-37, incorporated by reference herein in its entirety), or the ToLCDR2transposon system (see e.g., Tsukahara et al., Gene Ther. 2015 Feb; 22(2): 209-215, incorporated by reference herein in its entirety) in DNA, mRNA, or protein form. In some embodiments, the transposon and/or transposase of the transposon-based vector systems can be produced as a minicircle DNA vector before introduction into T cells and/or NK cells (see e.g., Hudecek et al., Recent Results Cancer Res. 2016;209:37-50 and Monjezi et al., Leukemia. 2017 Jan;31(1):186-194, incorporated by reference herein in their entireties). However, in some situations, the transposase-based carrier systems are not the preferred method of introducing an exogenous nucleic acid. Thus, in some embodiments, a polynucleotide of any of the aspects or embodiments disclosed herein does not include the transposon ITR fragments. In some embodiments, a modified, genetically modified, and/or transduced cell of any of the aspects or embodiments disclosed herein does not include the transposase carrier system as DNA or mRNA or protein.

The anti-idiotype polypeptide, CAR or lymphoproliferative element can also be integrated into the defined and specific sites in the genome using CRISPR or TALEN mediated integration, by adding 50-1000 bp homology arms homologous to the integration 5′ and 3′ of the target site (Jae Seong Lee et al. Scientific Reports 5, Article number: 8572 (2015), Site-specific integration in CHO cells mediated by CRISPR/Cas9 and homology-directed DNA repair pathway). CRISPR or TALEN provide specificity and genomic-targeted cleavage and the construct will be integrated via homology-mediated end joining (Yao X et al. Cell Res. 2017 Jun;27(6):801-814. doi: 10.1038/cr.2017.76. Epub 2017 May 19). The CRISPR or TALEN can be co-transfected with target plasmid as DNA, mRNA, or protein.

For any of the methods for modifying, genetically modifying, and/or transducing T and/or NK cells (e.g., in whole blood or in whole blood fractions such as TNCs or PBMCs), or uses that include such methods, or modified cells produced using such methods, and any other method or product-by-process provided herein, a skilled artisan will understand where an exogenous nucleic acid(s) could be introduced into the cells using methods that do not include a replication incompetent recombinant retroviral particle, for example using another type of recombinant vector (e.g. a plasmid associated with a lipid transfection agent).

### Inhibitory RNA Molecules

Embodiments of any of the aspects provided herein can include recombinant retroviral particles whose genomes are constructed to induce expression of one or more, and in illustrative embodiments two or more, inhibitory RNA molecules, such as for example, a miRNA or shRNA, after integration into a host cell, such as a lymphocyte (e.g., a T cell and/or an NK cell). Such inhibitory RNA molecules can be encoded within introns, including for example, an EF1-a intron. This takes advantage of the present teachings of methods to maximize the functional elements that can be included in a packageable retroviral genome to overcome shortcomings of prior teachings and maximize the effectiveness of such recombinant retroviral particles in adoptive T cell therapy.

In some embodiments, the inhibitory RNA molecule includes a 5′ strand and a 3′ strand (in some examples, sense strand and antisense strand) that are partially or fully complementary to one another such that the two strands are capable of forming a 18-25 nucleotide RNA duplex within a cellular environment. The 5′ strand can be 18, 19, 20, 21, 22, 23, 24, or 25 nucleotides in length, and the 3′ strand can be 18, 19, 20, 21, 22, 23, 24, or 25 nucleotides in length. The 5′ strand and the 3′ strand can be the same or different lengths, and the RNA duplex can include one or more mismatches. Alternatively, the RNA duplex has no mismatches. In some illustrative, a vector or genome herein, includes 2 or more, of the inhibitory RNA provided herein.

The inhibitory RNA molecules included in compositions and methods provided herein, in certain illustrative examples, do not exist and/or are not expressed naturally in T cells into whose genome they are inserted. In some embodiments, the inhibitory RNA molecule is a miRNA or an shRNA. In some embodiments, an inhibitory molecule of an embodiment of the present disclosure is a precursor of a miRNA, such as for example, a Pri-miRNA or a Pre-miRNA, or a precursor of an shRNA. In some embodiments, the miRNA or shRNA are artificially derived (i.e., artificial miRNAs or siRNAs). In other embodiments, the inhibitory RNA molecule is a dsRNA (either transcribed or artificially introduced) that is processed into an siRNA or the siRNA itself. In some embodiments, the miRNA or shRNA has a sequence that is not found in nature, or has at least one functional segment that is not found in nature, or has a combination of functional segments that are not found in nature.

In some embodiments, inhibitory RNA molecules are positioned in the first nucleic acid molecule in a series or multiplex arrangement such that multiple miRNA sequences are simultaneously expressed from a single polycistronic miRNA transcript. In some embodiments, the inhibitory RNA molecules can be adjoined to one another either directly or indirectly by non-functional linker sequence(s). The linker sequence in some embodiments, is between 5 and 120 nucleotides in length, and in some embodiments can be between 10 and 40 nucleotides in length, as non-limiting examples. In some embodiments, functional sequences can be expressed from the same transcript as the inhibitory RNA molecules, for example, any of the lymphoproliferative elements provided herein. In some embodiments, the inhibitory RNA molecule is a naturally occurring miRNA such as but not limited to miR-155, miR-30, miR-17-92, miR-122, and miR-21. In some embodiments, an inhibitory RNA molecule includes from 5′ to 3′ orientation: a 5′ microRNA flanking sequence, a 5′ stem, a loop, a 3′ stem that is partially or fully complementary to said 5′ stem, and a 3′ microRNA flanking sequence. In some embodiments, the 5′ stem (also called a 5′ arm herein) is 18, 19, 20, 21, 22, 23, 24 or 25 nucleotides in length. In some embodiments, the 3′ stem (also called a 3′ arm herein) is 18, 19, 20, 21, 22, 23, 24, or 25 nucleotides in length. In some embodiments, the loop is 3 to 40, 10 to 40, 20 to 40, or 20 to 30 nucleotides in length, and in illustrative embodiments the loop can be 18, 19, 20, 21, or 22 nucleotides in length. In some embodiments, one stem is two nucleotides longer than the other stem. The longer stem can be the 5′ or the 3′ stem. The inhibitory RNA molecule can be any of the inhibitory RNA molecules in the Inhibitory RNA Molecules section herein.

In some embodiments, the 5′ microRNA flanking sequence, 3′ microRNA flanking sequence, or both, are derived from a naturally occurring miRNA, such as but not limited to miR-155, miR-30, miR-17-92, miR-122, and miR-21. In certain embodiments, the 5′ microRNA flanking sequence, 3′ microRNA flanking sequence, or both, are derived from a miR-155, such as, e.g., the miR-155 from Mus musculus or Homo sapiens. Inserting a synthetic miRNA stem-loop into a miR-155 framework (i.e., the 5′ microRNA flanking sequence, the 3′ microRNA flanking sequence, and the loop between the miRNA 5′ and 3′ stems) is known to one of ordinary skill in the art (Chung, K. et al. 2006. Nucleic Acids Research. 34(7): e53; US 7,387,896) for example the SIBR and eSIBR sequences. In some embodiments of the present disclosure, miRNAs can be placed in the SIBR or eSIBR miR-155 framework. In illustrative embodiments herein, miRNAs are placed in a miR-155 framework that includes the 5′ microRNA flanking sequence of miR-155 represented by SEQ ID NO:333 or a functional variant thereof, the 3′ microRNA flanking sequence represented by SEQ ID NO:334 (nucleotides 221-265 of the Mus musculus BIC noncoding mRNA) or a functional variant thereof; and a modified miR-155 loop (SEQ ID NO:335) or a functional variant thereof. However, any known microRNA framework that is functional to provide proper processing within a cell of miRNAs inserted therein to form mature miRNA capable of inhibiting expression of a target mRNA to which they bind, is contemplated within the present disclosure.

In some embodiments, where two or more inhibitory RNA molecules (in some examples, 1, 2, 3, 4, 5, 6, 7, 8, 9, or 10 inhibitory RNA molecules) are included, these inhibitory RNA molecules are directed against the same or different RNA targets (such as e.g., mRNAs transcribed from genes of interest).

In some embodiments, the one or more inhibitor RNA molecules are one or more lymphoproliferative elements, accordingly, in any aspect or embodiment provided herein that includes a lymphoproliferative element, unless incompatible therewith, or already stated therein. In illustrative embodiments, miRNAs inserted into the genome of T cells in methods provided herein, are directed at targets such that proliferation of the T cells is induced and/or enhanced and/or apoptosis is suppressed. In some embodiments, the RNA targets are mRNAs transcribed from genes that are miR-155 targets.

In some embodiments, the inhibitory RNA, for example miRNA, targets mRNA encoding ABCG1, Cbl Proto-Oncogene (RNF55) (also known as cCBL and RNF55) (HGNC: 1541, Entrez Gene: 867, OMIM: 165360), T-Cell Receptor T3 Zeta Chain (CD3z) (HGNC: 1677, Entrez Gene: 919, OMIM: 186780), T cell receptor alpha locus (TCRA) (also known as TCRα) (HGNC: 12027, Entrez Gene: 6955, OMIM: 186880), T cell receptor beta locus (TCRB) (also known as TCRβ) (HGNC: 12155, Entrez Gene: 6957, OMIM: 186930), PD1, CTLA4, IFN gamma, T Cell Immunoglobulin Mucin 3 (TIM3) (also known as Hepatitis A Virus Cellular Receptor 2) (HGNC: 18437 Entrez Gene: 84868, OMIM: 606652), Lymphocyte Activating 3 (LAG3) (HGNC: 6476, Entrez Gene: 3902, OMIM: 153337), SMAD2, TNF Receptor Superfamily Member 10b (TNFRSF10B) (HGNC: 11905, Entrez Gene: 8795, OMIM: 603612), Protein Phosphatase 2 Catalytic Subunit Alpha (PPP2CA) (HGNC: 9299, Entrez Gene: 5515, OMIM: 176915), Tumor Necrosis Factor Receptor Superfamily Member 6 (TNFRSF6) (also known as Fas Cell Surface Death Receptor (FAS)) (HGNC: 11920, Entrez Gene: 355, OMIM: 134637), B And T Lymphocyte Associated (BTLA) (HGNC: 21087, Entrez Gene: 151888, OMIM: 607925), T Cell Immunoreceptor With Ig And ITIM Domains (TIGIT) (HGNC: 26838, Entrez Gene: 201633, OMIM: 612859), Adenosine A2a Receptor (ADORA2A or A2AR) (HGNC: 263, Entrez Gene: 135, OMIM: 102776), Aryl Hydrocarbon Receptor (AHR) (HGNC: 348, Entrez Gene: 196, OMIM: 600253), Eomesodermin (EOMES) (HGNC: 3372, Entrez Gene: 8320, OMIM: 604615), SMAD Family Member 3 (SMAD3) (HGNC: 6769, Entrez Gene: 4088, OMIM: 603109), SMAD Family Member 4 (SMAD4) (GNC: 6770, Entrez Gene: 4089, OMIM: 600993), TGFBR2, TRAIL2, PP2A, Protein Phosphatase 2 Regulatory Subunit B delta (PPP2R2D) (HGNC: 23732, Entrez Gene: 55844, OMIM: 613992), Tumor Necrosis Factor Ligand Superfamily Member 6 (TNFSF6) (also known as FASL) (HGNC: 11936, Entrez Gene: 356, OMIM: 134638), Caspase 3 (CASP3) HGNC: 1504, Entrez Gene: 836, OMIM: 600636), SOCS1, Suppressor Of Cytokine Signaling 2 (SOCS2) (HGNC: 19382, Entrez Gene: 8835, OMIM: 605117), Kruppel Like Factor 10 (KLF10) (also known as TGFB-Inducible Early Growth Response Protein 1 (TIEG1)) (HGNC: 11810, Entrez Gene: 7071, OMIM: 601878), JunB Proto-Oncogene, AP-1 Transcription Factor Subunit (JunB) (HGNC: 6205, Entrez Gene: 3726, OMIM: 165161), Chromobox 1 (Cbx) (HGNC: 1551, Entrez Gene: 10951, OMIM: 604511), Cbx3, Tet Methylcytosine Dioxygenase 2 (Tet2) (HGNC: 25941, Entrez Gene: 54790, OMIM: 612839), Hexokinase 2 (HK2) (HGNC: 4923, Entrez Gene: 3099, OMIM: 601125), Src homology region 2 domain-containing phosphatase-1 (SHP1) (HGNC: 9658, Entrez Gene: 5777, OMIM: 176883), Src homology region 2 domain-containing phosphatase-2 (SHP2) (HGNC: 9644, Entrez Gene: 5781, OMIM: 176876), colony stimulating factor 2 (CSF2; GMCSF) (Entrez Gene: 1437). In some embodiments, the inhibitory RNA, for example miRNA, targets the antigen that the ASTR of the CAR binds to.

In some aspects, provided herein is a polynucleotide designed to express a self-driving CAR. Details regarding such replication incompetent recombinant retroviral particles, and composition and method aspects including a self-driving CAR, are disclosed in more detail herein, for example in the Self-Driving CAR Methods and Compositions section and in the Exemplary Embodiments section. In some embodiments, the polynucleotides designed to express a self-driving CAR can include any of the inhibitory RNA molecules disclosed herein. Such polynucleotides can also have inhibitory RNA molecules that target inhibitors of the NFAT pathway, with or without the other inhibitory RNA molecules disclosed herein. In some embodiments, the inhibitory RNA molecules can target CABIN, Homer2, AKAP5, LRRK2, and/or DSCR1/MCIP (knockdown of the RNA molecules encoding these proteins can reduce inhibition of calcineurin or calmodulin); and/or Dyrk1A, CK1, and/or GSK3 (knockdown of the RNA molecules encoding these proteins can prevent phosphorylation, and nuclear export, of NFAT). In some further illustrative embodiments, a vector or genome herein, includes 2 or more, 2-10, 2-8, 2-6, 3-5, 2, 3, 4, 5, 6, 7, or 8 of the inhibitory RNA (e.g., miRNA) identified herein, for example in the paragraph immediately above.

In some embodiments provided herein, the two or more inhibitory RNA molecules can be delivered in a single intron, such as but not limited to EF1-a intron A. Intron sequences that can be used to harbor miRNAs for the present disclosure include any intron that is processed within a T cell. Sequence requirements for introns are known in the art. In some embodiments, such intron processing is operably linked to a riboswitch, such as any riboswitch disclosed herein. Accordingly, in illustrative embodiments provided herein is a combination of an miRNA directed against an endogenous T cell receptor subunit, wherein the expression of the miRNA is regulated by a riboswitch, which can be any of the riboswitches discussed herein.

In some embodiments, inhibitory RNA molecules can be provided on multiple nucleic acid sequences that can be included on the same or a different transcriptional unit. For example, a first nucleic acid sequence can encode one or more inhibitory RNA molecules and be expressed from a first promoter and a second nucleic acid sequence can encode one or more inhibitory RNA molecules and be expressed from a second promoter. In illustrative embodiments, two or more inhibitory RNA molecules are located on a first nucleic acid sequence that is expressed from a single promoter. The promoter used to express such miRNAs, are typically promoters that are inactive in a packaging cell used to express a retroviral particle that will deliver the miRNAs in its genome to a target T cell, but such promoter is active, either constitutively or in an inducible manner, within a T cell. The promoter can be a Pol I, Pol II, or Pol III promoter. In some illustrative embodiments, the promoter is a Pol II promoter.

### Characterization and Commercial Production Methods

The present disclosure provides various methods and compositions that can be used as research reagents in scientific experimentation and for commercial production. Such scientific experimentation can include methods for characterization of lymphocytes, such as NK cells and in illustrative embodiments, T cells using methods for modifying, for example genetically modifying and/or transducing lymphocytes provided herein. Such methods for example, can be used to study activation of lymphocytes and the detailed molecular mechanisms by which activation makes such cells transducible. Furthermore, provided herein are modified and in illustrative embodiments genetically modified lymphocytes that will have utility for example, as research tools to better understand factors that influence T cell proliferation and survival. In some embodiments, modified cells can express anti-idiotype polypeptides herein, which for example can serve as cell tags for identifying transduced cells. Such modified lymphocytes, such as NK cells and in illustrative embodiments T cells, can furthermore be used for commercial production, for example for the production of certain factors, such as growth factors and immunomodulatory agents, that can be harvested and tested or used in the production of commercial products.

The scientific experiments and/or the characterization of lymphocytes can include any of the aspects, embodiments, or subembodiments provided herein useful for analyzing or comparing lymphocytes. In some embodiments, T cells and/or NK cells can be transduced with the replication incompetent recombinant retroviral particles provided herein that include polynucleotides. In some embodiments, transduction of the T cells and/or NK cells can include polynucleotides that include polynucleotides encoding polypeptides of the present disclosure, for example, anti-idiotype polypeptides, CARs, lymphoproliferative elements, and/or activation elements. In some embodiments, the polynucleotides can include inhibitory RNA molecules as discussed elsewhere herein. In some embodiments, the lymphoproliferative elements can be chimeric lymphoproliferative elements.

### Exemplary Embodiments

Provided in this Exemplary Embodiments section are non-limiting exemplary aspects and embodiments provided herein and further discussed throughout this specification. For the sake of brevity and convenience, all of the aspects and embodiments disclosed herein and all of the possible combinations of the disclosed aspects and embodiments are not listed in this section. Additional embodiments and aspects are provided in other sections herein. Furthermore, it will be understood that embodiments are provided that are specific embodiments for many aspects, as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are discussed more specifically in other sections of this detailed description. Thus, for example any of the embodiments provided herein can be used in any of the polynucleotide, polynucleotide vector, method, use, reaction mixture, cell formulation, kit, cell processing assembly, filter assembly, modified, genetically modified, and transduced cell, e.g. T cell or NK cell, cell mixtures, or cell populations aspect provided herein, unless incompatible with, or otherwise stated.

Accordingly, in some aspects, provided herein is a polynucleotide, comprising nucleic acids encoding an anti-idiotype polypeptide comprising an anti-idiotype extracellular recognition domain (anti-id ERD) that recognizes an idiotype of a target antibody or a target antibody mimetic. A polynucleotide that includes nucleic acids encoding an anti-idiotype extracellular recognition domain (anti-id ERD) that recognizes an idiotype of a target antibody or a target antibody mimetic can be called herein, an anti-idiotype polynucleotide or an anti-idiotype encoding polynucleotide, or an anti-id polynuc. The anti-id ERD of an anti-id polynucleotide can be any of the anti-id ERDs disclosed herein, and the anti-idiotype polypeptide encoded by such anti-idiotype polynucleotide can be any of the anti-idiotype polypeptides disclosed herein, or encoded by any of the anti-idiotype polynucleotides herein. Such anti-idiotype polypeptides encoded by any of the anti-idiotype polypeptides herein, themselves are separate embodiments herein. In some embodiments, an anti-idiotype polynucleotide further comprises nucleic acids encoding a membrane association domain (MAD) and further in some embodiments, nucleic acids encoding a stalk. In some illustrative embodiments, the polynucleotide that encodes an anti-idiotype polypeptide can further includes nucleic acids encoding one or more inhibitory RNA molecules and/or a first engineered signaling polypeptide.

In one aspect, provided herein is a polynucleotide, comprising: one or more transcriptional units, wherein each of the one or more transcriptional units is operatively linked to a promoter wherein the one or more transcriptional units comprise:
a) nucleic acids encoding one or more inhibitory RNA molecules and/or a first engineered signaling polypeptide, and
b) nucleic acids encoding an anti-idiotype polypeptide comprising an extracellular recognition domain and a membrane association domain, wherein the extracellular recognition domain comprises an anti-idiotype domain that recognizes an idiotype of a target antibody or a target antibody mimetic.

In one aspect, provided herein is a polynucleotide, comprising: one or more transcriptional units, wherein each of the one or more transcriptional units is operatively linked to a promoter active in T cells and/or NK cells, wherein the one or more transcriptional units comprise:
a) nucleic acids encoding one or more inhibitory RNA molecules, lymphoproliferative elements, cytokines, chimeric antigen receptors (CARs), and recombinant T cell receptor (TCRs), and
b) nucleic acids encoding an anti-idiotype polypeptide comprising an anti-idiotype extracellular recognition domain that recognizes an idiotype of a target antibody or target antibody mimetic.

In one aspect, provided herein is a polynucleotide, comprising: one or more transcriptional units, wherein each of the one or more transcriptional units is operatively linked to a promoter active in T cells and/or NK cells, wherein the one or more transcriptional units comprise:
a) nucleic acids encoding one or more inhibitory RNA molecules and/or one or more of a lymphoproliferative element, a cytokine, a chimeric antigen receptor (CAR), and a recombinant T cell receptor (TCR), and
b) nucleic acids encoding an anti-idiotype polypeptide comprising an anti-idiotype extracellular recognition domain that recognizes an idiotype of a target antibody or target antibody mimetic, a membrane association domain, and a stalk that connects the anti-idiotype extracellular recognition domain to the membrane association domain.

In one aspect, provided herein is a polynucleotide, comprising: one or more transcriptional units, wherein each of the one or more transcriptional units is operatively linked to a promoter, wherein the one or more transcriptional units comprise:
a) nucleic acids encoding one or more inhibitory RNA molecules and/or one or more engineered signaling polypeptide selected from a lymphoproliferative element, a cytokine, a chimeric antigen receptor (CAR), and a recombinant T cell receptor (TCR), and
b) nucleic acids encoding an anti-idiotype polypeptide comprising an anti-idiotype extracellular recognition domain, a membrane association domain, and a stalk that connects the anti-idiotype extracellular recognition domain to the membrane association domain, wherein the anti-idiotype extracellular recognition domain comprises an idiotype-binding variable region of an anti-idiotype antibody or antibody mimetic that recognizes the idiotype of a target antibody or a target antibody mimetic.

In one aspect, provided herein is a polynucleotide, comprising: one or more transcriptional units, wherein each of the one or more transcriptional units is operatively linked to a promoter active in T cells and/or NK cells, wherein the one or more transcriptional units comprise:
a) nucleic acids encoding one or more engineered signaling polypeptide, wherein a first engineered signaling polypeptide of the one or more engineered signaling polypeptides, is a chimeric antigen receptor (CAR), and
b) nucleic acids encoding an anti-idiotype polypeptide comprising an anti-idiotype extracellular recognition domain, a membrane association domain, and a stalk that connects the anti-idiotype extracellular recognition domain to the membrane association domain, wherein the anti-idiotype extracellular recognition domain comprises an idiotype-binding variable region of an anti-idiotype antibody or antibody mimetic that recognizes the idiotype of a target antibody or a target antibody mimetic.

In some aspects, provided herein is a modified cell, which can be an isolated cell, and in illustrative embodiments is a mammalian cell, such as a human cell for example, comprising any of the polynucleotides disclosed herein, and in illustrative embodiments expressing the polynucleotides. In illustrative embodiments, the polynucleotide comprises nucleic acids encoding an anti-idiotype polypeptide. Such anti-idiotype polypeptide can be according to any anti-idiotype polypeptide embodiment provided herein. In some embodiments, the cell is a primary cell. In some embodiments, the cell is not from a cell line. In some embodiments, the cell is not an immortalized cell. A some embodiments, the cell is not an immortalized cell. In some embodiments, the cell is an ex-vivo cultured cell. In some embodiments, the cell is a type of cell or a type of lymphocyte that in its native form does not produce antibodies. In some embodiments, the cell is a TIL. In some embodiments, the cell is a T cell or an NK cell, a CAR-T cell, or a CAR-NK cell, or a population of T cells and/or NK cells, or a population of CAR-T cells and/or CAR-NK cells, or a population of any of the aforementioned cells in this paragraph and in throughout this disclosure. In some embodiments, the population of cells, such as human cells, are within a vessel for gene therapy or cell therapy, such as an infusion bag. In some embodiments at least 5%, 10%, 20%, 25%, 30%, 40%, 50%, 60%, 70%, or 75% of the modified cells of the population comprise the polynucleotide, and in illustrative embodiments express the anti-idiotype polypeptide. In some embodiments between 1%, 2%, 5%, 10%, 20%, or 25% on the low end of the range, and 50%, 60%, 70%, 75%, 80%, 90%, 95%, 99%, or 100% on the high end of the range of the modified cells of the population, comprise an anti-idiotype polynucleotide, and in illustrative embodiments express the anti-idiotypte polypeptide.

In some aspects, provided herein is a method for delivering to a subject, modified cells, typically mammalian cells, for example lymphocytes, primary cells, primary lymphocytes, lymphocytes that are of a type that do not naturally express an antibody, and in illustrative embodiments, T cells and/or NK cells, wherein the modified mammalian cells are modified to include any of the polynucleotides disclosed herein. In illustrative embodiments, the polynucleotide includes nucleic acids encoding an anti-idiotype polypeptide, and thus typically encodes an anti-id ERD. The method typically comprises administering the modified cells, e.g. T cells and/or NK cells, to the subject. In some embodiments, the methods are methods for treating a disorder, such as a hyperproliferative disorder, for example cancer. In some embodiments, such methods further include instructing a user to deliver the target antibody or antibody mimetic to the subject if treatment for adverse events in the subject is warranted. For example, such instructions could indicate that treatment is warranted if a subject experiences grade 3 or grade 4 adverse events for cytokine release syndrome or IEC-associated neurotoxicity syndrome. In some embodiments, such methods further include administering the target antibody to the subject, for example after the subject develops adverse events, as provided herein. In some embodiments, the target antibody or the antibody mimetic is delivered in sufficient quantity to selectively kill at least 1%, 10%, 15%, 20%, 25%, 30%, 40%, 50%, 60%, 70%, 75%, 80%, 90%, 95%, or 99% of the genetically modified T cells and/or NK cells., thereby selectively killing the genetically modified T cells and/or NK cells. Such methods herein can be considered methods for selectively killing target modified cells.

In some aspects, provided herein is use of modified cells, in the manufacture of a kit, wherein the use of the kit comprises performing the method for delivering to the subject, or any of the methods disclosed herein. Especially contemplated for such use aspects, are any methods disclosed herein that involve delivering or administering modified cells to a subject.

In some aspects, provided herein is a polynucleotide vector comprising an anti-idiotype polynucleotide according to any aspect or embodiment herein. In some aspects, polynucleotides herein that include nucleic acids that encode an anti-idiotype polypeptide, comprise a promoter to drive expression of the nucleic acids encoding the anti-idiotype polypeptide. Tis promoter, or another promoter can be used to drive expression from other nucleic acids on the polynucleotide. In any of the aspects herein that are directed to, or include a promoter, the promoter can be a promoter active in mammalian cells, for example active in, or specifically active in lymphocytes. As another example, the promoter can be a promoter that is active in, or specifically active in T cells and/or NK cells.

In some embodiments of any of the polynucleotide or vector embodiments herein, the vector is a viral vector, and in further embodiments, is replication incompetent. In some embodiments, the viral vector is a retroviral particle. In some embodiments, the surface of the retroviral particle comprises a membrane-bound cytokine. In some embodiments, the membrane-bound cytokine is a chemokine. In some sub-embodiments, the vector comprises a cleavage signal effective for cleaving the membrane-bound cytokine from the membrane. In some embodiments, the retroviral particle is a lentiviral vector. In some embodiments, the viral vector further comprises an activation element on the surface of the retroviral particle, wherein the activation element is fused to a heterologous membrane attachment sequence, and wherein the activation element is a polypeptide capable of binding to CD3 or a nucleic acid (e.g.) aptamer capable of binding to CD3, on the surface of a resting T cell and activating the resting T cell, and wherein the activation element is not encoded by a polynucleotide in the retroviral particle. In some embodiments, the activation element comprises an anti-CD3 antibody or antibody mimetic. In some embodiments, the viral particle comprises a means on its surface for binding to CD3 on the surface of T cells.

In one aspect, provided herein is an anti-idiotype polypeptide, that is encoded by any of the polynucleotides provided herein that comprise nucleic acids encoding an anti-id ERD. In one aspect, provided herein is a polypeptide, comprising an extracellular recognition domain, a membrane association domain, and a stalk connecting the extracellular recognition domain to the membrane association domain, and further comprising an intracellular domain (ICD) of a first engineered signaling polypeptide, wherein the first engineered signaling polypeptide is a chimeric antigen receptor (CAR), a recombinant T cell receptor (TCR), or a lymphoproliferative element (LE), and wherein the extracellular recognition domain an anti-idiotype extracellular recognition domain that recognizes an idiotype of a target antibody.

In some non-limiting embodiments, an anti-idiotype polypeptide comprises an extracellular recognition domain, a membrane association domain, and a stalk connecting the extracellular recognition domain to the membrane association domain, and further comprising an intracellular domain of a first engineered signaling polypeptide, wherein the extracellular recognition domain comprises a domain that recognizes an idiotype of a target antibody. In some embodiments, the first engineered signaling polypeptide is a lymphoproliferative element (LE), a cytokine, a chimeric antigen receptor (CAR), and a recombinant T cell receptor (TCR).

In some non-limiting embodiments, an anti-idiotype polypeptide comprises an extracellular recognition domain, an intracellular domain, a membrane association domain, and a stalk connecting the extracellular recognition domain to the membrane association domain, wherein the extracellular recognition domain comprises a domain that recognizes an idiotype of a target antibody.

In illustrative embodiments of any aspect herein that includes an anti-idiotype extracellular recognition domain, the anti-idiotype extracellular recognition domain comprises an idiotype-binding variable region of an anti-idiotype antibody or an idiotype-binding region of an anti-idiotype antibody mimetic. In some embodiments, the anti-idiotype extracellular recognition domain is an antibody selected from a single-chain antibody, an Fab fragment, an Fab′ fragment, an (Fab′)2 fragment, an Fv fragment, an scFv, a divalent single-chain antibody, a diabody, an scFv-Fc, an scF-CH, an scFab, or an scFv-zipper. In certain illustrative embodiments, the anti-idiotype extracellular recognition domain is an antibody comprising an scFV. In some embodiments, the idiotype-binding variable regions comprise a framework region, and wherein the framework region is a human framework region.

In some embodiments of any of the aspects and embodiments herein that include a polynucleotide, including a polynucleotide vector, that includes nucleic acids encoding an anti-idiotype polypeptide that includes an ICD, or embodiments directed to the polypeptide encoded by such nucleic acids, or a cell that includes and optionally expresses the polynucleotides, the polynucleotide further encodes a second engineered signaling polypeptide, which in certain illustrative embodiments is a lymphoproliferative element. In some embodiments, the lymphoproliferative element comprises an intracellular domain that is a means for transmitting a signal that promotes proliferation or survival of a T cell and/or NK cell. In some embodiments, the lymphoproliferative element is constitutively active. In some embodiments, the polynucleotide comprises an internal ribosome entry site (IRES), a ribosomal skip sequence and/or cleavage signal, between nucleic acids encoding the first engineered signaling polypeptide or the second engineered signaling polypeptide, and nucleic acids encoding the anti-idiotype polypeptide. In some embodiments, the polynucleotide comprises an internal ribosome entry site (IRES), a ribosomal skip sequence and/or cleavage signal, between nucleic acids encoding the second engineered signaling polypeptide, and nucleic acids encoding the anti-idiotype polypeptide.

In some embodiments that include a polynucleotide that includes nucleic acids that encode an anti-idiotype polypeptide, a CAR and an LE, the anti-idiotype polypeptide, the CAR, and the LE can be expressed as one, two, or three separate polypeptides.

In some embodiments for any aspect herein that include an anti-Id ERD, the target antibody or antibody mimetic that is recognized by the anti-id ERD is a clinical antibody or clinical antibody mimetic that is the subject of an FDA-approved Investigational New Drug Application (IND), or equivalent approved regulatory filing for initial clinical testing in humans in another country or jurisdiction. In some embodiments for any aspect herein, the target antibody or antibody mimetic that is recognized by the anti-id ERD is a therapeutic antibody or a therapeutic antibody mimetic, respectively. In some embodiments for any of these embodiments, as per the IND or equivalent is a stand-alone product, with no other active therapeutic or ingredient being tested as part of the IND. In some embodiments for any aspect herein, the target antibody or antibody mimetic that is recognized by the anti-id ERD has been shown in one or more clinical trials to have an acceptable safety (i.e. adverse event) profile. In some embodiments for any aspect herein, the target antibody or antibody mimetic that is recognized by the anti-id ERD is a clinical antibody or clinical antibody mimetic that has passed human clinical safety testing in a stand-alone clinical trial of the clinical antibody or antibody mimetic. In some embodiments for any aspect herein, the target antibody or antibody mimetic that is recognized by the anti-id ERD is a clinical antibody or antibody mimetic for which a stand-alone application for regulatory approval has been filed with the Food And Drug Administration of the U.S. (USFDA), European Medicines Agency (EMA), National Medical Products Administration of China (NMPA) (Chinese FDA), or the Pharmaceutical and Food Safety Bureau (PFSB) of Japan. In some embodiments for any aspect herein, the target antibody or antibody mimetic that is recognized by the anti-id ERD is a clinical antibody or antibody mimetic for which an application for approval (e.g. Biologic License Application (BLA)) has been filed with the Food And Drug Administration of the U.S. (USFDA), European Medicines Agency (EMA), National Medical Products Administration of China (NMPA) (Chinese FDA), or the Pharmaceutical and Food Safety Bureau (PFSB) of Japan. In some illustrative embodiments, the target antibody or target antibody mimetic is an approved biologic antibody or antibody mimetic, approved by the Food And Drug Administration of the U.S. (USFDA), European Medicines Agency (EMA), National Medical Products Administration of China (NMPA) (Chinese FDA), or the Pharmaceutical and Food Safety Bureau (PFSB) of Japan. In some embodiments, the approved biologic antibody or antibody mimetic is the approved biologic antibody, and wherein the approved biologic antibody is cetuximab, muromonab-CD3, efalizumab, tositumomab-i131, nebacumab, edrecolomab, catumaxomab, daclizumab, olaratumab, abciximab, rituximab, basiliximab, palivizumab, infliximab, trastuzumab, adalimumab, ibritumomab tiuxetan, omalizumab, bevacizumab, natalizumab, panitumumab, ranibizumab, eculizumab, certolizumab pegol, ustekinumab, canakinumab, golimumab, ofatumumab, tocilizumab, denosumab, belimumab, ipilimumab, brentuximab vedotin, pertuzumab, ado-trastuzumab emtansine, raxibacumab, obinutuzumab, siltuximab, ramucirumab, vedolizumab, nivolumab, pembrolizumab, blinatumomab, alemtuzumab, evolocumab, idarucizumab, necitumumab, dinutuximab, secukinumab, mepolizumab, alirocumab, daratumumab, elotuzumab, ixekizumab, reslizumab, bezlotoxumab, atezolizumab, obiltoxaximab, brodalumab, dupilumab, inotuzumab ozogamicin, guselkumab, sarilumab, avelumab, emicizumab, ocrelizumab, benralizumab, durvalumab, gemtuzumab ozogamicin, erenumab (erenumab-aooe), galcanezumab (galcanezumab-gnlm), burosumab (burosumab-twza), lanadelumab (lanadelumab-flyo), mogamulizumab (mogamulizumabkpkc), tildrakizumab (tildrakizumab-asmn), fremanezumab (fremanezumab-vfrm), ravulizumab (ravulizumab-cwvz), cemiplimab (cemiplimab-rwlc), ibalizumab (ibalizumab-uiyk) emapalumab (emapalumab-lzsg), moxetumomab pasudotox (moxetumomab pasudotox-tdfk), caplacizumab (caplacizumab-yhdp), risankizumab (risankizumab-rzaa), polatuzumab vedotin (polatuzumab vedotinpiiq), romosozumab (romosozumab-aqqg), brolucizumab (brolucizumab-dbll), crizanlizumab (crizanlizumab-tmca), enfortumab vedotin (enfortumab vedotin-ejfv), [fam-]trastuzumab deruxtecan (fam-trastuzumab deruxtecan-nxki), teprotumumab (teprotumumab-trbw), eptinezumab (eptinezumabjjmr), isatuximab (isatuximab-irfc), sacituzumab govitecan (sacituzumab govitecan-hziy), inebilizumab (inebilizumab-cdon), tafasitamab (tafasitamab-cxix), belantamab mafodotin (belantamab mafodotinblmf), satralizumab (satralizumab-mwge), atoltivimab, maftivimab, odesivimab-ebgn, naxitamab-gqgk, margetuximab-cmkb, ansuvimab-zykl, evinacumab, dostarlimab (dostarlimab-gxly), loncastuximab tesirine (loncastuximab tesirine-lpyl), amivantamab (amivantamab-vmjw), aducanumab (aducanumabavwa), tralokinumab, anifrolumab (anifrolumab-fnia), oportuzumab monatox, tisotumab vedotin, bimekizumab, narsoplimab, tezepelumab, sintilimab, inolimomb, balstilimab, ublituximab, toripalimab, omburtamab, penpulimab, tanezumab, faricimab, sutimlimab, teplizumab, and retifanlimab.

In some embodiments for any aspect herein that include an anti-Id ERD, the anti-idiotype polypeptide comprises a means for binding an idiotype of cetuximab. In some embodiments, the approved biologic antibody is cetuximab. In some embodiments, the anti-idiotype polypeptide is capable of binding to cetuximab, and the anti-id ERD comprises any of the sequences provided herein for such cetuximab anti-idiotype ERD. In some embodiments, the anti-idiotype polypeptide is capable of blocking binding of cetuximab to epidermal growth factor receptor. In some embodiments, the target antibody comprises an antigen-binding site, and the anti-idiotype polypeptide is capable of binding to the antigen-binding site of the target antibody.

In some embodiments of any of the aspects and embodiments herein that include a polynucleotide, including a polynucleotide vector, that includes nucleic acids encoding an anti-idiotype polypeptide, or embodiments directed to the polypeptide encoded by such nucleic acids, or a cell that includes and optionally expresses the polynucleotides, the membrane association domain is a Gpi anchor.

In some embodiments of any of the aspects and embodiments herein that include a polynucleotide, including a polynucleotide vector, that includes nucleic acids encoding an anti-idiotype polypeptide, or embodiments directed to the polypeptide encoded by such nucleic acids, or a cell that includes and optionally expresses the polynucleotides, the anti-idiotype polypeptide further comprises one or more intracellular domains (ICD). In some embodiments, the ICD is between 1 and 35 amino acids in length. In some embodiments, the ICD is between 10 and 250 amino acids in length.

In some exemplary embodiments the ICD has primarily or exclusively a structural function, and/or in related embodiments does not comprise a signaling domain, or does not comprise a proliferation, survival, and/or apoptotic signaling domain. In some exemplary embodiments the ICD comprises a signaling domain, which in illustrative embodiments is a proliferation, survival, and/or apoptotic signaling domain.

In some embodiments and subembodiments wherein the anti-Idiotype polypeptide comprises an ICD, the membrane association domain is a transmembrane domain. The combination of stalk (sometimes referred to as a hinge), transmembrane domain, and ICD can be referred to herein as a STMICD. In some embodiments, the STMICD of an anti-idiotype polypeptide comprises the stalk, TM and 8 amino acids of the ICD of PDGFRβ and comprises a sequence as provided in SEQ ID NO: 676. In some embodiments, the STMICD of an anti-idiotype polypeptide comprised the stalk, TM and 9 amino acids of the ICD of CD28 and comprises the sequence provided in SEQ ID NO: 677. In some embodiments, the STMICD of an anti-idiotype polypeptide comprises the hinge, TM and the full ICD of CD28 and comprises the seuquence provided in SEQ ID NO: 678. In some embodiments, the STMICD of an anti-idiotype polypeptide comprises the hinge, TM, and full ICD of CD28 fused to the full ICD of CD80 and comprises the sequence provided in SEQ ID NO: 679.

In some embodiments, in illustrative embodiments wherein the membrane association domain is a transmembrane domain, the ICD is the ICD of an LE, and in further illustrative embodiments of these embodiments, the transmembrane domain is from an LE. In some embodiments, the ICD comprises all or part of an intracellular signaling domain of one or more cytokine receptors, and wherein the ICD is capable of activating a signaling pathway. In some embodiments, the inducible lymphoproliferative element is capable of activating a proliferative or survival signaling after binding of the anti-idiotype polypeptide to the target antibody or target antibody mimetic. In some embodiments, the ICD is capable upon dimerization, of activating a Jak/Stat pathway, a TRAF pathway, a PI3K pathway, and/or a PLC pathway. In some embodiments, the one or more cytokine receptors are selected from CD27, CD40, CRLF2, CSF2RA, CSF2RB, CSF3R, EPOR, GHR, IFNAR1, IFNAR2, IFNGR1, IFNGR2, IFNLR1, IL1R1, IL1RAP, IL1RL1, IL1RL2, IL2R, IL2RA, IL2RB, IL2RG, IL3RA, IL4R, IL5RA, IL6R, IL6ST, IL7R, IL7RA, IL9R, IL10RA, IL10RB, IL11RA, IL12RB1, IL13R, IL13RA1, IL13RA2, IL15R, IL15RA, IL17RA, IL17RB, IL17RC, IL17RE, IL18R1, IL18RAP, IL20RA, IL20RB, IL21R, IL22RA1, IL23R, IL27R, IL27RA, IL31RA, LEPR, LIFR, MPL, OSMR, PRLR, TGFβR, TGFβ decoy receptor, TNFRSF4, TNFRSF8, TNFRSF9, TNFRSF14, or TNFRSF18.

In some embodiments of any of the aspects and embodiments herein that include a polynucleotide, including a polynucleotide vector, that includes nucleic acids encoding an anti-idiotype polypeptide, or embodiments directed to the polypeptide encoded by such nucleic acids, or a cell that includes and optionally expresses the polynucleotides, the membrane association domain is the transmembrane domain of the lymphoproliferative element, the CAR, or the recombinant TCR. In some embodiments of any aspect herein, the transmembrane domain is from BAFFR, C3Z, CEACAM1, CD2, CD3A, CD3B, CD3D, CD3E, CD3G, CD3Z, CD4, CD5, CD7, CD8A, CD8B, CD9, CD11A, CD11B, CD11C, CD11D, CD27, CD16, CD18, CD19, CD22, CD28, CD29, CD33, CD37, CD40, CD45, CD49A, CD49D, CD49F, CD64, CD79A, CD79B, CD80, CD84, CD86, CD96 (Tactile), CD100 (SEMA4D), CD103, C134, CD137, CD154, CD160 (BY55), CD162 (SELPLG), CD226 (DNAM1), CD229 (Ly9), CD247, CRLF2, CRTAM, CSF2RA, CSF2RB, CSF3R, EPOR, FCER1G, FCGR2C, FCGRA2, GHR, HVEM (LIGHTR), IA4, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL7RA Ins PPCL, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA1, ITGA4, ITGA6, ITGAD, ITGAE, ITGAL, ITGAM, ITGAX, ITGB1, ITGB2, ITGB7, KIRDS2, LEPR, LFA-1 (CD11a, CD18), LIFR, LTBR, MPL, NKp80 (KLRF1), OSMR, PAG/Cbp, PRLR, PSGL1, SLAM (SLAMF1, CD150, IPO-3), SLAMF4 (CD244, 2B4), SLAMF6 (NTB-A, Ly108), SLAMF7, SLAMF8 (BLAME), TNFR2, TNFRSF4, TNFRSF8, TNFRSF9, TNFRSF14, TNFRSF18, VLA1, or VLA-6, or functional mutants and/or fragments thereof. In some embodiments of any aspect herein, the transmembrane domain comprises a polypeptide sequence with at least 50%, 60%, 70%, 75%, 80%, 85%, 90%, 95%, 96%, 97%, 98%, 99% or 100% sequence identity to a stretch of at least 10, 15, 20, or all of the amino acids of one or more of the following: CD8 alpha TM (SEQ ID NO:17); CD8 beta TM (SEQ ID NO:18); CD4 stalk (SEQ ID NO:19); CD3Z TM (SEQ ID NO:20); CD28 TM (SEQ ID NO:21); CD134 (OX40) TM: (SEQ ID NO:22); CD7 TM (SEQ ID NO:23); CD8 stalk and TM (SEQ ID NO:24); and CD28 stalk and TM (SEQ ID NO:25). In some embodiments of any aspect herein, the transmembrane domain is derived from an antibody. In some embodiments of any aspect herein, the transmembrane domain is at least 75, 80, 85, 90, 95, or 100% identical to the transmembrane domain of IgD. In some embodiments of this embodiment, the polynucleotide further comprises a stretch of at least 24, 48, 60, or 99 nucleotides encoding all or a fragment of IgA and IgB. In some subembodiments, of the embodiments above, the transmembrane domain is at least 75, 80, 90, 95, or 100% identical to the transmembrane domain from IgD.

In some embodiments of any of the aspects and embodiments herein that include a polynucleotide, including a polynucleotide vector, that includes nucleic acids encoding an anti-idiotype polypeptide, or embodiments directed to the polypeptide encoded by such nucleic acids, or a cell that includes and optionally expresses the polynucleotides, the of the anti-idiotype polypeptide is between 4 and 250, 4 and 200, or 4 and 100 amino acids in length. In some embodiments of any aspect herein, the stalk domain comprises a polypeptide sequence with at least 85%, 90%, 95%, 96%, 97%, 98%, 99% or 100% sequence identity to a stretch of at least 10, 15, 20, or all of the amino acids of one or more of the following amino acid sequences: TTTPAPRPPTPAPTIASQPLSLRPEACRPAAGGAVHTRGLDFA (SEQ ID NO:2), FCKIEVMYPPPYLDNEKSNGTIIHVKGKHLCPSPLFPGPSKP (SEQ ID NO:3), CPPC (SEQ ID NO:4), DKTHT (SEQ ID NO:5); CPEPKSCDTPPPCPR (SEQ ID NO:6), ELKTPLGDTTHT (SEQ ID NO:7), KSCDKTHTCP (SEQ ID NO:8), KCCVDCP (SEQ ID NO:9), KYGPPCP (SEQ ID NO:10), EPKSCDKTHTCPPCP (SEQ ID NO:11), ERKCCVECPPCP (SEQ ID NO:12), ELKTPLGDTTHTCPRCP (SEQ ID NO:13), SPNMVPHAHHAQ (SEQ ID NO:14), EPKSCDKTYTCPPCP (SEQ ID NO:15), TTTPAPRPPTPAPTIASQPLSLRPEACRPAAGGAVHTRGLDFACD (SEQ ID NO:16).

In some embodiments of any aspect herein, the stalk domain is derived from an antibody. In some embodiments, the stalk domain comprises a heavy chain Fc domain. In some embodiments, the stalk domain encodes a dimerizing moiety, wherein the dimerizing moiety is constitutively dimerized. In some embodiments, the constitutively dimerized, dimerization domain comprises a leucine zipper.

In some embodiments of any of the aspects and embodiments herein that include a polynucleotide, including a polynucleotide vector, that includes nucleic acids encoding an anti-idiotype polypeptide, or embodiments directed to the polypeptide encoded by such nucleic acids, or a cell that includes and optionally expresses the polynucleotides, a polynucleotide herein that includes nucleic acids that encode and anti-id ERD furthermore includes nucleic acids that encode one or more inhibitory RNA molecules. In some embodiments, the one or more inhibitory RNA molecules reduce cytokine release syndrome or enhance proliferation. In some embodiments, the one of the one or more inhibitory RNA molecules targets IFN gamma.

In some embodiments of any of the aspects and embodiments herein that include a polynucleotide, including a polynucleotide vector, that includes nucleic acids encoding an anti-idiotype polypeptide, or embodiments directed to the polypeptide encoded by such nucleic acids, or a cell that includes and optionally expresses the polynucleotides, the target antibody is structured such that binding of the anti-idiotype polypeptide to the target antibody induces antibody-dependent cellular cytotoxicity. In some embodiments of any of the aspects and embodiments herein that include an anti-id ERD, or nucleic acids encoding the same, the target antibody is structured such that binding of the anti-idiotype polypeptide to the target antibody induces complement-dependent cytotoxicity. In some embodiments of any of the aspects and embodiments herein that include an anti-id ERD, or nucleic acids encoding the same, the target antibody is structured such that binding of the anti-idiotype polypeptide to the target antibody induces opsonization. In some embodiments of any of the aspects and embodiments herein that include an anti-id ERD, or nucleic acids encoding the same, the target antibody is structured such that binding of the anti-idiotype polypeptide to the target antibody does not induce antibody-dependent cellular cytotoxicity.

In some embodiments of any of the aspects and embodiments herein that include a polynucleotide, including a polynucleotide vector, that includes nucleic acids encoding an anti-idiotype polypeptide that includes an ICD, or embodiments directed to the polypeptide encoded by such nucleic acids, or a cell that includes and optionally expresses the polynucleotides, the ICD comprises an intracellular apoptosis domain capable of inducing or promoting or transmitting an apoptotic signal. In some subembodiments of these embodiments, the target antibody is not capable of, structured to, configured to, or adapted to induce cytotoxicity. In some subembodiments of these embodiments, the target antibody is capable of, structured to, configured to, or adapted to induce cytotoxicity.

In some embodiments of any of the aspects and embodiments herein that include a polynucleotide, including a polynucleotide vector, that includes nucleic acids encoding an anti-idiotype polypeptide that includes an ICD, or embodiments directed to the polypeptide encoded by such nucleic acids, or a cell that includes and optionally expresses the polynucleotides, the ICD comprises an intracellular apoptosis domain capable of inducing an apoptotic signal upon binding of the anti-idiotype extracellular recognition domain to a target antibody or antibody mimetic comprising the idiotype. In some subembodiments of these embodiments, the anti-idiotype polypeptide further comprises a proteolytic cleavage site as part of and/or between the transmembrane domain and the intracellular apoptosis domain, wherein the proteolytic cleavage site has a cleavage site amino acid sequence such that the anti-idiotype polypeptide is cleaved at the proteolytic cleavage site when the anti-idiotype polypeptide is dimerized by binding of a target antibody or antibody mimetic to the anti-idiotype extracellular recognition domain. In further subembodiments of these embodiments, the proteolytic cleavage site is effective for cleavage by a gamma-secretase complex or a Notch receptor upon dimerization of the anti-idiotype polypeptide. In further subembodiments of these embodiments, the proteolytic cleavage site is effective for cleavage by a gamma-secretase complex or a Notch receptor upon dimerization of the anti-idiotype polypeptide. In further subembodiments of these embodiments, the intracellular apoptosis domain comprises one or more of a Caspase 2 polypeptide, a Caspase 8 polypeptide, a Caspase 9 polypeptide, and a Caspase 10 polypeptide, wherein such intracellular apoptosis domain is capable of activating an effector caspase upon cleavage from the anti-idiotype polypeptide. In further subembodiments of these embodiments, the intracellular apoptosis domain comprises one or more caspase polypeptides, wherein the caspase polypeptides are amino acids 327 to 452 of SEQ ID NO:680 (Caspase 2), amino acids 384 to 496 of SEQ ID NO:681 (Caspase 8), amino acids 294 to 416 of SEQ ID NO:682 (Caspase 9), and amino acids 365 to 478 of SEQ ID NO:683 (Caspase 10). In further subembodiments of these embodiments, the intracellular apoptosis domain comprises one or more caspase polypeptides, wherein the caspase polypeptides are each a polypeptide that when dimerized has initiator caspase function and is at least 90% identical to amino acids 327 to 452 of SEQ ID NO:680 (Caspase 2), amino acids 384 to 496 of SEQ ID NO:681 (Caspase 8), amino acids 294 to 416 of SEQ ID NO:682 (Caspase 9), and amino acids 365 to 478 of SEQ ID NO:683 (Caspase 10).

In further subembodiments of these embodiments where the ICD comprises an intracellular apoptosis domain, including certain embodiments wherein the anti-idiotype polypeptide comprises a protease cleavage site, and other embodiments wherein the anti-idiotype polypeptide does not comprise a protease cleavage site, for example that is activated by binding of an anti-id ERD to a target antibody, the ICD comprises one or more caspase activation and recruitment domains (CARDs), death domains (DDs), death effector domains (DEDs), pyrin domains (PYDs), and/or caspase proteolytic domains that are activate to transmit an apoptosis-inducing signal upon dimerization. For example, these domains could be from a caspase, for example an initiator caspase, for example caspase 2, 8, 9, or 10. In some subembodiments of these embodiments, the one or more CARDS, DDs, DEDs, PYDs, and/or caspase proteolytic domains comprises one or more CARDs from Apaf-1, DARK, CED-4, CED-3, Dronc, CARMA1, Bcl-10, Nod1, Nod2, RIP2, ICEBERG, RIG-I, MDA5, MAV5, ASC, NALP1, caspase 1, caspase 2, caspase-5, and/or caspase 9. In some subembodiments of these embodiments, the intracellular apoptotic domain can include one or more DDs from TNF-R1, Fas, p75, TRADD, FADD, RIP, MyD88, IRAKs, Pelle, Tube, PIDD, RAIDD, and/or MALT1, and/or functional fragments thereof. In some subembodiments of these embodiments, the intracellular apoptotic domain can include one or more DEDs from FADD, caspase 8, caspase 10, c-FLIP, v-FLIPs, MC159, PEA-15, DEDD, and/or DEDD2, and/or functional fragments thereof. In some subembodiments of these embodiments, the intracellular apoptotic domain can include one or more PYDs from ASC, ASC2, NALP1, NALP1, NALP3, NALP4, NALP5, NALP6, NALP7, NALP8, NALP9, NALP10, NALP11, and/or NALP12, and/or functional fragments thereof.

In some embodiments of any of the aspects and embodiments herein that include a polynucleotide, including a polynucleotide vector, that includes nucleic acids encoding an anti-idiotype polypeptide that includes an ICD, or embodiments directed to the polypeptide encoded by such nucleic acids, or a cell that includes and optionally expresses the polynucleotides, the ICD comprises a dimerizing moiety, wherein the dimerizing moiety is constitutively dimerized, and/or wherein the target antibody is an IgM antibody. In some subembodiments of these embodiments and the other embodiments herein wherein the ICD comprises an apoptosis domain, the intracellular apoptosis domain comprises one or more death domains (DD) from FAS, TNF-R1, DR3, DR4, and/or DR5.

In some embodiments of any of the aspects and embodiments herein that include a polynucleotide, including a polynucleotide vector, that includes nucleic acids encoding an anti-idiotype polypeptide that includes an ICD, or embodiments directed to the polypeptide encoded by such nucleic acids, or a cell that includes and optionally expresses the polynucleotides, wherein the membrane association domain is a transmembrane domain, the transmembrane domain is the transmembrane domain of the CAR and the ICD is the ICD of a CAR. In some subembodiments of these and other embodiments, the CAR is a bispecific CAR, wherein one ASTR of the CAR comprises the anti-idiotype extracellular recognition domain. In some subembodiments of these and other embodiments, another ASTR of the CAR is capable of binding to a tumor-associated antigen or tumor-specific antigen. In some subembodiments of these and other embodiments, the one ASTR and the other ASTR of the CAR are scFV antibodies. In some subembodiments of these and other embodiments, the one ASTR and the other ASTR of the CAR are scFV antibodies. the one ASTR and the other ASTR of the CAR comprise an scFv-FC, scFv-CH, and scFv-zipper antibody.

In one aspect, provided herein is a retroviral particle, comprising:
a) a polynucleotide comprising nucleic acids encoding an anti-idiotype polypeptide comprising an anti-idiotype extracellular recognition domain, a membrane association domain, and a stalk that connects the anti-idiotype extracellular recognition domain to the membrane association domain, wherein the anti-idiotype extracellular recognition domain comprises an idiotype-binding variable region of an anti-idiotype antibody or antibody mimetic that recognizes the idiotype of a target antibody or a target antibody mimetic; and
b) an activation element on the surface of the retroviral particle, wherein the activation element is fused to a heterologous membrane attachment sequence, and wherein the activation element is a polypeptide capable of binding to CD3 on the surface of a resting T cell and activating the resting T cell, and is not encoded by a polynucleotide in the retroviral particle.

In some embodiments of any aspect or embodiment herein that includes an anti-id ERD or a polynucleotide with nucleic acids encoding the same, the idiotype is present on two target antibodies, a first target antibody, which promotes cytotoxicity, and a second target antibody, which promote less cytotoxicity than the first target antibody. In some embodiments wherein the idiotype is present on two target antibodies, the two target antibodies differ in their glycosylation patterns. In some embodiments, including embodiments wherein the idiotype is present on two target antibodies, the target antibody, or one or both of the target antibodies comprises glycosylated residues. In some embodiments of any aspect herein, the idiotype recognized by the anti-id ERD comprises glycosylated residues. In some embodiments, including embodiments wherein the idiotype is present on two target antibodies, the target antibody or one or both of the target antibodies comprises an α-Gal epitope. In some embodiments, including embodiments wherein the idiotype is present on two target antibodies, the target antibody comprises one or more glycoforms, or the target antibodies are different glycoforms. In some embodiments, the glycoforms comprise the target antibody with an α-1,3-Gal residue. In some embodiments, the glycoforms comprise the target antibody with an N-glycolylneuraminic acid residue. In some embodiments, the glycoforms comprise the target antibody with an oligomannose.

In some embodiments, the target antibody is produced by a cell line such that the target antibody is glycosylated. In some embodiments, including embodiments wherein the idiotype is present on two target antibodies,,the target antibody is produced in SP2/0 cells, and/or the two target antibodies are made in different cell lines. In some embodiments, including embodiments wherein the idiotype is present on two target antibodies, the target antibody is produced in NS0 cells and the second target antibody is made in a different cell line. In some embodiments, including embodiments wherein the idiotype is present on two target antibodies, the target antibody is produced in Chinese hamster ovary (CHO) cells and the second target antibody is made in a different cell line.

In one aspect, provided herein is a method for delivering modified T cells and/or NK cells to a subject, the method comprising administering the modified cells to the subject, wherein the modified cells are modified with a polynucleotide comprising nucleic acids encoding an anti-idiotype polypeptide and nucleic acids encoding a CAR.

In one aspect, provided herein is use of modified T cells and/or NK cells in the manufacture of a kit, wherein the use of the kit comprises: administering the modified T cells and/or NK cells to a subject, wherein the modified T cells and/or NK cells comprise a polynucleotide comprising nucleic acids encoding an anti-idiotype polypeptide and nucleic acids encoding a CAR.

In one aspect, provided herein is a population of modified cells, in illustrative embodiments modified T cells and/or NK cells, wherein at least some modified cells of the population of cells comprise a polynucleotide comprising nucleic acids encoding an anti-idiotype polypeptide and nucleic acids encoding a CAR.

In one illustrative embodiment, provided herein is a population of modified cells, in illustrative embodiments modified T cells and/or NK cells, wherein at least some of the modified cells of the population of modified cells comprise a polynucleotide comprising nucleic acids encoding an anti-idiotype polypeptide, and in illustrative embodiments express the anti-idiotype polypeptide. In some embodiments of this illustrative embodiment, and other embodiments herein that include a population of cells, at least 5%, 10%, 20%, 25%, 30%, 40%, 50%, 60%, 70%, or 75% of the modified cells of the population comprise the polynucleotide, and in illustrative embodiments express the anti-idiotype polypeptide. In some embodiments between 1%, 2%, 5%, 10%, 20%, or 25% on the low end of the range, and 50%, 60%, 70%, 75%, 80%, 90%, 95%, 99%, or 100% on the high end of the range of the modified cells of the population, comprise the polynucleotide, and in illustrative embodiments express the anti-idiotypte polypeptide. In some embodiments of this illustrative embodiment, and any other embodiment herein, the modified cells of the population are primary cells, ex-vivo cultured cells, not cells derived from a cell line, and/or not immortalized cells. In some exemplary embodiments of this illustrative embodiment, the anti-idiotype polypeptide comprises a membrane association domain, which in some illustrative embodiments is a transmembrane domain.

In some exemplary embodiments of the illustrative embodiment immediately above, and any other embodiment or aspect herein, the anti-idiotype polypeptide lacks an intracellular domain (ICD). In some exemplary embodiments of the illustrative embodiment immediately above, and any other embodiment or aspect herein, the anti-idiotype polypeptide comprises an ICD. In some exemplary embodiments the ICD has primarily or exclusively a structural function, and/or in related embodiments does not comprise a signaling domain, or does not comprise a proliferation, survival, and/or apoptotic signaling domain. In some exemplary embodiments the ICD comprises a signaling domain, which in illustrative embodiments is a proliferation, survival, and/or apoptotic signaling domain. The anti-idiotype polypeptides and polypeptides that comprise nucleic acids encoding the same, themselves represent separate illustrative embodiments herein, regardless of whether they are within a cell or not within a cell.

In one aspect, provided herein is a polypeptide, or polynucleotide with nucleic acids encoding the same, comprising a lymphoproliferative element according to any embodiment herein, wherein the extracellular domain comprises an anti-id ERD. In one aspect, provided herein is a polypeptide, or polynucleotide with nucleic acids encoding the same, comprising a chimeric antigen receptor according to any embodiment herein, wherein an ASTR of the CAR comprises an anti-id ERD.

The inventors have observed that delivery of modified T cells and/or NK cells subcatenously can form subcutaneous lymphoid structures. Accordingly, in another aspect, provided herein is a subcutaneous lymphoid structure, which can be considered a tertiary lymphoid structure, that comprises at least some of the modified lymphocytes of a population of modified lymphocytes, and in illustrative embodiments, genetically modified lymphocytes, that include a polynucleotide comprising nucleic acids encoding an anti-idiotype polypeptide and typically one or more of a CAR or a TCR, and in illustrative embodiments one or more of an LE or a cytokine. In some embodiments, some of the genetically modified lymphocytes expressing the CAR are located in lymphatic vasculature. In some embodiments, the other white blood cells comprise B cells, macrophages, dendritic cells, T cells and/or NK cells. In some embodiments, some of the modified lymphocytes of the population are in lymphatic vasculature localized near, in certain embodiments within 25, 50, 75, 100, 125, 150, 200, 250, 500, or 1,000 µm from, the subcutaneous lymphoid structure. In some embodiments, the subcutaneous lymphoid structure or the population of genetically modified lymphocytes further comprises actively dividing lymphocytes that are native to the subject and do not express the CAR. In some embodiments, the genetically modified lymphocytes express a lymphoproliferative element. In some embodiments, the subcutaneous lymphoid structure is an artificial lymph node. In some embodiments, the population of genetically modified lymphocytes is in an artificial lymph node.

In some embodiments, the T cells comprises CD4+ and CD8+ cells, and wherein at least 50% of the genetically modified lymphocytes that are CD4+ and/or CD8+ are CD3-;

In some embodiments of any of the population of genetically modified lymphocytes aspects or embodiments herein,
i) the population of genetically modified lymphocytes comprises a persisting population of genetically modified lymphocytes expressing the transgene, the engineered T cell receptor, or the chimeric antigen receptor (CAR), that persists in the subject for at least 7, 14, 21, or 28 days or 1, 2, 3, 4, 5, 6, 7, 8, 9, 10, 11, or 12 months or 1, 2, 3, 4, or 5 years after administration.
ii) the genetically modified lymphocytes produce a population of progeny lymphocytes, wherein the population of progeny lymphocytes comprises at least 1 × 10⁵, 1 × 10⁶, 1 × 10⁷, 1 × 10⁸,1 × 10°, 1 × 10¹⁰, or 1 × 10¹¹, or between 1 × 10⁶ and 1 × 10¹⁰, or between 1 × 10⁸ and 1 × 10¹² cells;
iii) the population of genetically modified lymphocytes comprises at least 100 genetically modified lymphocytes localized subcutaneously and the subcutaneous region contains no artificial matrix components; and/or
iv) the at least 10 genetically modified lymphocytes of the population remain localized subcutaneously for at least 7, 14, 21, or 28 days, and in illustrative embodiments, the subcutaneous region contains no artificial matrix components.

In some embodiments, at least 1 × 10⁵, 1 × 10⁶, 1 × 10⁷, 1 × 10⁸,1 × 10⁹, 1 × 10¹⁰, or 1 × 10¹¹, or between 1 × 10⁶ and 1 × 10¹⁰, or between 1 × 10⁸ and 1 × 10¹² cells of the genetically modified lymphocytes are located subcutaneously. In some embodiments, at least 1 × 10⁵, 1 × 10⁶, 1 × 10⁷, 1 × 10⁸,1 × 10⁹, 1 × 10¹⁰, ort 1 × 10¹¹, or between 1 × 10⁶ and 1 × 10¹⁰, or between 1 × 10⁸ and 1 × 10¹² cells are not in the subcutaneous region, and in illustrative embodiments are circulating in the blood and/or at the site of a tumor in the subject.

In one aspect, provided herein is a subcutaneous lymphoid structure, comprising: cell aggregates, wherein the cell aggregates comprise:
a) at least 10, 100, 1 × 10³, 1 × 10⁴, 1 × 10⁵, 1 × 10⁶, 1 × 10⁷, 1 × 10⁸,1 × 10⁹, 1 × 10¹⁰, or 1 × 10¹¹ genetically modified lymphocytes expressing an anti-idiotype polypeptide, in illustrative embodiments and antigen, an engineered T cell receptor, or a chimeric antigen receptor (CAR), wherein the cell aggregates are localized subcutaneously in a subject, and wherein the genetically modified lymphocytes comprise T cells and/or NK cells; and
b) other white blood cells that do not express the CAR, wherein at least 10% of the cells in the cell aggregates are other white blood cells.

In any of the aspects provided herein that include intramuscular, and in illustrative embodiments subcutaneous administration of modified lymphocytes (e.g. modified T cells and/or NK cells), such method can in certain embodiments, include a step wherein the modified cells expand subcutaneously (e.g. expanding the modified cells subcutaneously), for example at or near (e.g. within 10, 5, 4, 3, 2, or 1 cm) a site of subcutaneous administration, for days (e.g., for up to 5, 7, 14, 17, 21, or 28 days) or months (e.g., for up to 1, 2, 3, 6, 12, or 24 months). In some embodiments of aspects herein that include intraperitoneal, intramuscular, and in illustrative embodiments subcutaneous administration of modified T cells and/or NK cells, or RIPs to modify T cells and/or NK cells in vivo, the modified T cells and/or NK cells (e.g., genetically modified T cells and/or NK cells) migrate away from the site of subcutaneous administration to other sites of the body, for example to tumors. Thus, in some embodiments modified and in illustrative embodiments, such methods can include a step wherein genetically modified T cells and/or NK cells appear in circulation migrating away from a subcutaneous administration site, days (e.g., 1, 2, 3, 4, 5, 6, or 7 days), weeks (e.g., 1, 2, 4, or 4 weeks), or months (e.g., 1, 2, 3, 6, 12, or 24 months) after the modified T cells are injected intraperitoneally, intramuscularly, or in illustrative embodiments subcutaneously into a subject. In certain embodiments, at these timepoints, such methods can include a step wherein an area, or in illustrative embodiments a concentration gradient of modified, and in illustrative embodiments genetically modified, T cells and/or NK cells forms emanating from the site of intramuscular, or in illustrative embodiments subcutaneous administration.

In any of the aspects provided herein that include intraperitoneal, intramuscular, and in illustrative embodiments subcutaneous administration of modified lymphocytes (e.g. modified T cells and/or NK cells), certain embodiments can include a step of delivery of another component(s) such as a molecule(s) (ion(s)), macromolecule(s) (e.g. DNA, RNA, peptides, and polypeptides) and/or other cell(s), such as other modified cell(s) (e.g. genetically modified cell(s)), that can affect the modified T cells and/or NK cells, subcutaneously at or near the site of delivery of the modified T cells and/or NK cells, in the same or a different formulation. In certain illustrative embodiments, the other component(s) include an antigen, a recombinant cell encoding a recombinant antigen, or an RNA encoding the antigen, or a cytokine that drives proliferation of T cells and/or NK cells. These other components, which are disclosed in more detail herein, can be delivered either in the same formulation or in different formulation(s) than the modified T cells and/or NK cells. Furthermore, these other components can be delivered along with the modified T cells and/or NK cells or can be delivered days (e.g., 1, 2, 3, 4, 5, 6, or 7 days), weeks (e.g., 1, 2, 4, or 4 weeks), or even months (e.g., 1, 2, 3, 6, 12, or 24 months) before or after the modified T cells and/or NK cells are delivered. In some embodiments, one or more of these other components is delivered at more than one time point, such as on the same day as, or simultaneously with the modified T cells and/or NK cells, and at one or more of the times recited hereinabove in this paragraph. Accordingly, in some embodiments a second formulation is administered to the subject at a second timepoint between 1 day and 1 month, 2 months, 3 months, 6 months, or 12 months after the administering the cell formulation. The other component that is administered to the subject in addition to modified lymphocytes or substantially purified or purified RIPs, can include a cytokine, for example IL-2, ii) an antibody or polypeptide that is capable of binding CD2, CD3, CD28, OX40, 4-1BB, ICOS, CD9, CD53, CD63, CD81, and/or CD82, and/or iii) a source of the cognate antigen recognized by the CAR). In certain embodiments subcutaneous administration of the modified T cells and/or NK cells is performed near (e.g., within 1, 1, 2, 3, 4, 5, 10, 20, or 30 cm) a site of neoplastic (e.g., cancerous) cells, such as a tumor, or an organ comprising a tumor, including for example, the spleen in the case of blood cancers, or where multiple administrations are performed of the same formulation, or of different formulations, they can be performed at or near the site of a prior administration or away from such site. In some embodiments, the cell formulation comprises a source of a cognate antigen for the CAR, wherein the source of the cognate antigen is the cognate antigen, an mRNA encoding the cognate antigen, or a cell expressing the cognate antigen. In some embodiments, the cell formulation comprises a cytokine and wherein the cytokine is IL-2, IL-7, IL-15, or IL-21 or a modified version of any of these cytokines that is capable of binding to and activating a native receptor for the cytokine. The cognate antigen for this and any embodiments herein, including in this Exemplary Embodiments section, can be any of the tumor associated or tumor specific antigens provided herein.

Provided herein in one aspect is a cell formulation, comprising modified T cells and/or NK cells, wherein the modified T cells and/or NK cells are suspended in a delivery solution and are either or both:
i) genetically modified with a polynucleotide comprising one or more transcriptional units, wherein each of the one or more transcriptional units is operatively linked to a promoter active in T cells and/or NK cells, or
ii) associated with a RIP comprising the polynucleotide,

wherein the one or more transcriptional units encode an anti-idiotype polypeptide, and in illustrative embodiments a first polypeptide comprising a CAR, and wherein the cell formulation in illustrative embodiments is contained within a syringe, and has a volume of between 0.5 ml and 20 ml, or 2 ml and 10 ml, or another subcutaneous or intramuscular cell formulation volume provided herein, and further comprises at least one of, for example two or more of, neutrophils, B cells, monocytes, basophils, and eosinophils. In illustrative embodiments, the cell formulation is compatible with, effective for, and/or adapted for intramuscular delivery and in further illustrative embodiments subcutaneous delivery.

In some embodiments of any of the aspects that are or include a cell formulations herein, and any reaction mixture embodiments, especially that that include a subcutaneous, intramuscular reaction, or intraperitoneal reaction mixture, the cell formulation or the reaction mixture further comprises i) a cytokine, ii) an antibody, antibody mimetic, or polypeptide that is capable of binding CD3, CD28, OX40, 4-1BB, ICOS, CD9, CD53, CD63, CD81, and/or CD82, and/or iii) a source of the cognate antigen recognized by the CAR.

Additional cell formulation aspects and embodiments are provided below and in the Detailed Description herein, outside this Exemplary Embodiments section. Various volumes of cell formulations are provided herein for any cell formulation aspect. In some embodiments, the cell formulation is 3 ml or greater in volume, for example 3 ml to 600 ml in volume, or between 50 ml and 500 ml, or between 100 ml and 500 ml. In some embodiments, the cell formulation comprises hyaluronidase. In some embodiments, the cell formulation is 1 ml to 10 ml, 1 ml to 5 ml, 1 ml to 3 ml or 10, 5, 4, 3, or 2 ml or less, or less than 3 ml, or any of the Small Volume Elements provided herein. In illustrative embodiments, the cell formulation does not comprise hyaluronidase. Other volumes and formulations are provided herein. In some embodiments for any of the cell formulation aspects herein, the cell formulation is contained within a syringe. In some embodiments, the cell formulation, for any cell formulation provided herein, is in an incubation bag or a blood processing bag. In illustrative embodiments, the syringe is made using Good Manufacturing Practice (GMP) and is GMP grade and quality.

Provided herein in another aspect is a method for modifying, genetically modifying, and/or transducing a lymphocyte (e.g. a T cell or an NK cell) or a population thereof, comprising contacting blood cells comprising the lymphocyte (e.g. the T cell or NK cell) or the population thereof, ex vivo with a RIP comprising in its genome a polynucleotide comprising one or more nucleic acid sequences operatively linked to a promoter active in lymphocytes (e.g. T cells and/or NK cells), wherein a first nucleic acid sequence of the one or more nucleic acid sequences encodes an anti-idiotype polypeptide, and one or more of a CAR comprising an ASTR, a transmembrane domain, and an intracellular activating domain, one or more (e.g. two or more) inhibitory RNA molecules directed against one or more RNA targets, and a polypeptide lymphoproliferative element, wherein said contacting facilitates genetic modification and/or transduction of the lymphocyte (e.g. T cell or NK cell), or at least some of the lymphocytes (e.g. T cells and/or NK cells) by the RIP, thereby producing a modified, genetically modified, and/or transduced lymphocyte (e.g. T cell and/or NK cell). In such method, the contacting is typically performed in a reaction mixture, sometimes referred to herein as a transduction reaction mixture, comprising a population of lymphocytes (e.g., T cells and/or NK cells) and contacted with a population of RIPs. Various contacting times are provided herein, including, but not limited to, in this Exemplary Embodiments section, that can be used in this aspect to facilitate membrane association, and eventual membrane fusion of the lymphocytes (e.g., T cells and/or the NK cells) to the RIPs. In an illustrative embodiment, contacting is performed for less than 15 minutes. Provided herein in one aspect, is use of RIPs in the manufacture of a kit for modifying lymphocytes (e.g. T cells or NK cells) of a subject, wherein the use of the kit comprises the aspect disclosed above in this paragraph, and any relevant embodiments herein.

In some embodiments the method can further include introducing the modified T cell and/or NK cell into a subject. In illustrative embodiments, the blood cells comprising the lymphocyte (e.g., the T cell and/or NK cell) are from the subject, and thus the introducing is a reintroducing. In this aspect, in some embodiments, a population of lymphocytes (e.g., T cells and/or NK cells) are contacted in the contacting step, modified, genetically modified, and/or transduced, and introduced into the subject in the introducing step.

Provided herein in another aspect is the use of RIPs in the manufacture of a medicament for modifying lymphocytes, for example T cells and/or NK cells of a subject, wherein the use of the medicament comprises the method aspect provided hereinabove in this paragraph.

In another aspect, provided herein is kit for modifying NK cells and/or T cells, comprising:

one or a plurality of first containers containing polynucleotides, typically substantially pure polynucleotides (e.g., found within recombinant retroviral particles according to any embodiment herein), comprising a first transcriptional unit operatively linked to a promoter active in T cells and/or NK cells, wherein the first transcriptional unit encodes a first polypeptide comprising an anti-idiotype polypeptide, and one or more of a CAR; a cytokine and a lymphoproliferative element.

Provided in the following paragraphs, are exemplary aspects and embodiments that can be used in or combined with any aspect or embodiment provided herein unless incompatible with or otherwise indicated, as will be recognized by a skilled artisan. In another aspect, provided herein is a modified, in illustrative embodiments genetically modified, and in further illustrative embodiments stably transfected or stably transcribed lymphocyte(s) (e.g., T cell(s) or NK cell(s)) made by modifying lymphocytes (e.g., T cells and/or NK cells) according to any method herein.

In another aspect, provided herein is use of RIPs in a kit, or in the manufacture of a kit, for modifying T cells and/or NK cells of a subject, wherein the use of the kit comprises any of the methods for delivering cells or modifying T cells and/or NK cells provided herein. In another aspect, provided herein is use of a RIPs in a kit, or in the manufacture of a kit for delivering to a subject, administering to a subject, injecting into a subject, and/or engrafting in a subject, modified lymphocytes, wherein the use of the kit comprises any of the methods for delivering to a subject, administering to a subject, injecting into a subject, and/or engrafting in a subject, provided herein. In another aspect, provided herein is use of a RIPs in a kit, or in the manufacture of a kit for preparing a cell formulation, wherein the use of the kit comprises any of the methods for preparing a cell formulation comprising modifying T cells and/or NK cells provided herein. Provided herein in another aspect, are RIPs for use in subcutaneous delivery to a subject, wherein the use of the RIPs comprises any method provided herein, for subcutaneous delivery that comprises RIPs.

Provided in the following paragraphs, are exemplary embodiments, for example exemplary ranges and lists, that can be used for any of the aspects provided immediately above or otherwise herein, unless incompatible with or otherwise indicated, as will be recognized by a skilled artisan. Additional aspects and embodiments are provided in this specification outside this Exemplary Embodiments section.

In any of the aspects herein, the cell(s) or lymphocyte(s) is an NK cell(s) or in illustrative embodiments a T cell(s). It will be understood that in aspects that include collecting blood that such method can include collecting a blood-derived product or a peripheral blood-derived product, which can be a blood sample, such as an unfractionated blood sample, or can include blood cells (e.g., leukocytes or lymphocytes) collected by apheresis.

In any of the aspects herein that include a polynucleotide including one or more transcriptional units, the one or more transcriptional units can encode a polypeptide comprising an LE. In some embodiments, the extracellular domain of the LE is an anti-idiotype polypeptide. In some embodiments, the extracellular domain of the LE further comprises any of the dimerizing moieties disclosed herein, which in some embodiments can be considered a stalk domain. In some embodiments, the dimerizing motif can be a leucine zipper motif-containing polypeptide, CD69, CD71, CD72, CD96, Cd105, Cd161, Cd162, Cd249, CD271, or Cd324, as well as mutants and/or active fragments thereof that retain the ability to dimerize.

In some embodiments, the lymphoproliferative element comprises an intracellular signaling domain from a cytokine receptor, which in illustrative embodiments activates a Janus kinase/ Signal Transducer and Activator of Transcription (JAK/ STAT) pathway and/or a tumor necrosis factor receptor (TNF-R)-associated factor (TRAF) pathway. In illustrative embodiments, the lymphoproliferative element is constitutively active typically because it constitutively activates one or more signaling pathways. In illustrative embodiments, the lymphoproliferative element comprises Box1 and optionally Box2 JAK-binding motifs, and a STAT-binding motif comprising a tyrosine residue. In some illustrative embodiments, the lymphoproliferative element does not comprise an extracellular ligand binding domain or a small molecule binding domain. In some embodiments, the constitutively active signaling pathways include activation of PI3K pathways. In some embodiments, the constitutively active signaling pathways include activation of PLC pathways. Thus, in certain embodiments, lymphoproliferative elements for use in any of the kits, methods, uses, or compositions herein, are constitutively active and comprise an intracellular signaling domain that activates a Jak/Stat pathway a TRAF pathway, a PI3K pathway, and/or a PLC pathway. Any of the polypeptide lymphoproliferative elements disclosed herein, for example, but not limited to those disclosed in the “Lymphoproliferative elements” section herein, or functional mutants and/or fragments thereof, can be encoded. In some embodiments, the LE comprises a domain with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids or an intracellular domain from 4-1BB (CD137), B7-H3, B7-HCDR3, BAFFR, BTLA, C100 (SEMA4D), CD2, CD3D, CD3E, CD3G, CD4, CD7, CD8A, CD8B, CD11A, CD11B, CD11C, CD11D, CD18, CD19, CD27, CD28, CD28 deleted for Lck binding (ICΔ), CD29, CD30, CD40, CD49A, CD49D, CD49F, CD69, CD79A, CD79B, CD84, CD96 (Tactile), CD103, CD160 (BY55), CD162 (SELPLG), CD226 (DNAM1), CD229 (Ly9), a ligand that specifically binds with CD83, CDS, CEACAM1, CRLF2, CRTAM, CSF2RA, CSF2RB, CSF3R, EPOR, Fc receptor gamma chain, Fc receptor ε chain, FCER1G, FCGR2C, FCGRA2, GADS, GHR, GITR, HVEM, IA4, ICAM-1, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA4, ITGA6, ITGAD, ITGAE, ITGAL, ITGAM, ITGAX, ITGB1, ITGB2, ITGB7, LAT, LEPR, LFA-1 (CD11a/CD18), LIGHT, LIFR, LMP1, LTBR, MPL, MYD88, NKG2C, NKP80 (KLRF1), OSMR, OX40, PD-1, PRLR, PSGL1, PAG/Cbp, SLAM (SLAMF1, CD150, IPO-3), SLAMF4 (C244, 2B4), SLAMF6 (NTB-A, Ly108), SLAMF7, SLAMF8 (BLAME), SLP-76, TILR2, TILR4, TILR7, TILR9, TNFR2, TNFRSF4, TNFRSF8, TNFRSF9, TNFRSF14, TNFRSF18, TRANCE/RANKL, VLA1, or VLA-6,or functional mutants and/or fragments thereof, or functional mutants and/or fragments thereof. In any of the embodiments disclosed herein, the lymphoproliferative element can include an extracellular ligand binding domain or a small molecule binding domain. In some embodiments, the lymphoproliferative element can include a transmembrane domain. In some embodiments, the transmembrane domain can include a transmembrane domain from BAFFR, C3Z, CEACAM1, CD2, CD3A, CD3B, CD3D, CD3E, CD3G, CD3Z, CD4, CD5, CD7, CD8A, CD8B, CD9, CD11A, CD11B, CD11C, CD11D, CD27, CD16, CD18, CD19, CD22, CD28, CD29, CD33, CD37, CD40, CD45, CD49A, CD49D, CD49F, CD64, CD79A, CD79B, CD80, CD84, CD86, CD96 (Tactile), CD100 (SEMA4D), CD103, C134, CD137, CD154, CD160 (BY55), CD162 (SELPLG), CD226 (DNAM1), CD229 (Ly9), CD247, CRLF2, CRTAM, CSF2RA, CSF2RB, CSF3R, EPOR, FCER1G, FCGR2C, FCGRA2, GHR, HVEM (LIGHTR), IA4, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL7RA Ins PPCL, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA1, ITGA4, ITGA6, ITGAD, ITGAE, ITGAL, ITGAM, ITGAX, ITGB1, ITGB2, ITGB7, KIRDS2, LEPR, LFA-1 (CD11a, CD18), LIFR, LTBR, MPL, NKp80 (KLRF1), OSMR, PAG/Cbp, PRLR, PSGL1, SLAM (SLAMF1, CD150, IPO-3), SLAMF4 (CD244, 2B4), SLAMF6 (NTB-A, Ly108), SLAMF7, SLAMF8 (BLAME), TNFR2, TNFRSF4, TNFRSF8, TNFRSF9, TNFRSF14, TNFRSF18, VLA1, or VLA-6, or functional mutants and/or fragments thereof.

In some embodiments of any aspect herein that include RIPs, the RIPs can comprise a binding polypeptide and a fusogenic element, and can include a polynucleotide, which in illustrative embodiments is the genome of the RIP, that encodes any of the anti-idiotype polypeptides disclosed herein. In some embodiments, a polynucleotide in the RIP encodes an anti-idiotype polypeptide provided herein, which typically further encodes a CAR, a lymphoproliferative element, one or more inhibitory RNA molecules and/or a cytokine. In some embodiments, a polynucleotide in the RIP encodes an anti-idiotype polypeptide provided herein and a CAR, and optionally further encodes a lymphoproliferative element, one or more inhibitory RNA molecules and/or a cytokine. In some embodiments, one or more viral envelope proteins comprise the binding polypeptide and the fusogenic element. In some embodiments, a viral envelope protein is a mutated viral envelope protein wherein a binding polypeptide of the viral envelope protein has been mutated to reduce/eliminate binding to a target cell (e.g. a T cell), but wherein such binding is provided by another (e.g. a heterologous) binding polypeptide which in further illustrative embodiments is also an activation element as provided herein (e.g. a polypeptide that binds CD3). In some embodiments, the viral envelope protein comprises the feline endogenous virus (RD114) envelope protein, an oncoretroviral amphotropic envelope protein, an oncoretroviral ecotropic envelope protein, the vesicular stomatitis virus envelope protein (VSV-G), the baboon retroviral envelope glycoprotein (BaEV), the murine leukemia envelope protein (MuLV), the influenza glycoprotein HA surface glycoprotein (HA), the influenza glycoprotein neurominidase (NA), the paramyxovirus Measles envelope protein H, the paramyxovirus Measles envelope protein F, the Tupaia paramyxovirus (TPMV) envelope protein H, the TPMV envelope protein F, the Nipah virus (NiV) envelope protein F, the NiV envelope protein G, the Sindbis virus (SINV) protein E1, the SINV protein E2, and/or functional variants or fragments of any of these envelope proteins. In some embodiments, the viral envelope protein is the NiV envelope protein G, wherein the NiV envelope protein G comprises one or more mutations in residues Y389, E501, W504, E505, V507, Q530, E533, or I588 of SEQ ID NO:375. In some embodiments, Henipavirus-G protein is SEQ ID NO:375 with mutations E533A and/or Q530A. In some embodiments, one or more N- or O-glycosylation sites are mutated to improve pseudotyping and fusion. In some embodiments, one or more N-glycosylation sites are mutated for example, but not limited to, at one or more of N72, N159, N306, N378, N417, N481, or N529 of SEQ ID NO:375, or the corresponding glutamines of other Henipavirus-G proteins, to another amino acid such as glutamine. In some embodiments, one or more O-glycosylation sites are mutated from serine or threonine to another amino acid such as alanine. In some embodiments, one or more of the serine or threonine residues in the heavily O-glycosylated stalk domain from amino acids 103 to 137 of SEQ ID NO:375, is mutated to, for example, alanine. In other embodiments, the C-terminus of the Henipavirus-G protein can be modified and fused to a binding polypeptide and in illustrative embodiments, an activation element, such as an antibody or antibody mimetic, which in illustrative embodiments can be an anti-CD3 antibody or antibody mimetic.

In any of the aspects and embodiments provided herein that include a RIP, the RIP comprises a pseudotyping element on its surface that is capable of binding to a T cell and/or NK cell and facilitating membrane fusion of the RIP thereto. In some embodiments, the pseudotyping element is a viral envelope protein. In some embodiments, the viral envelope protein is one or more of the feline endogenous virus (RD114) envelope protein, the oncoretroviral amphotropic envelope protein, the oncoretroviral ecotropic envelope protein, the vesicular stomatitis virus envelope protein (VSV-G), the baboon retroviral envelope glycoprotein (BaEV), the murine leukemia envelope protein (MuLV), and/or the paramyxovirus Measles envelope proteins H and F, the Tupaia paramyxovirus (TPMV) envelope protein H, the TPMV envelope protein F, the Nipah virus (NiV) envelope protein F, the NiV envelope protein G, the Sindbis virus (SINV) protein E1, the SINV protein E2, or a fragment of any thereof that retains the ability to bind to resting T cells and/or resting NK cells. In illustrative embodiments, the pseudotyping element is VSV-G. As discussed elsewhere herein, the pseudotyping element can include a fusion with a T cell activation element, which in illustrative embodiments, can be a fusion with any of the envelope protein pseudotyping elements, for example MuLV or VSV-G, with an anti-CD3 antibody. In further illustrative embodiments, the pseudotyping elements include both a VSV-G and a fusion of an antiCD3scFv to MuLV.

In any of the aspects herein that include a RIP, the RIP(s) can comprise an activation element on their surface. In some embodiments, the activation element on the surface is a membrane-bound T cell activation element. In some embodiments, the activation element is a polypeptide capable of binding to a polypeptide on the surface of a lymphocyte, and in illustrative embodiments, a T cell and/or an NK cell. In some subembodiments of these and embodiments of any of the aspects provided herein,

In some embodiments, the T cell activation element comprises one or more of an antibody or an antibody mimetic capable of binding CD28, CD3, TCR α/β, CD28, or a mitogenic tetraspanin, or wherein said T cell activation element is a mitogenic tetraspanin. In some embodiments, the T cell activation element comprises the antibody or the antibody mimetic capable of binding CD3, and wherein the T cell activation element is bound to the membrane of the RIPs. In some embodiments, the membrane-bound anti-CD3 antibody or anti-CD3 antibody mimetic is an anti-CD3 scFv, an anti-CD3 scFvFc, or an anti-CD3 DARPin. In some embodiments, the anti-CD3 antibody or anti-CD3 antibody mimetic is bound to the membrane by a GPI anchor, such as a heterologous GPI anchor attachment sequence, wherein the anti-CD3 antibody or anti-CD3 antibody mimetic is a recombinant fusion protein with a MuLV viral envelope protein, with or without a mutation at a furin cleavage site, or wherein the anti-CD3 antibody or anti-CD3 antibody mimetic is a recombinant fusion protein with a VSV viral envelope protein, or wherein the anti-CD3 antibody or anti-CD3 antibody mimetic is a recombinant fusion protein with a Henipavirus-G envelope protein, or wherein the anti-CD3 antibody is a recombinant fusion protein with a NiV viral envelope protein. In some embodiments, the polypeptide capable of binding CD28 is CD80, or an extracellular domain thereof, bound to a CD16B GPI anchor attachment sequence.

In illustrative embodiments, the activation element is a T cell activation element capable of binding to a TCR complex polypeptide. In some embodiments, a TCR complex polypeptide is CD3D, CD3E, CD3G, CD3Z, TCRα, or TCRβ. In some embodiments, the activation element capable of binding to the TCR complex polypeptide is a polypeptide capable of binding to one or more of CD3D, CD3E, CD3G, CD3Z, TCRα, or TCRβ. In illustrative embodiments, the activation element activates ZAP-70. In some embodiments, the activation element includes a polypeptide capable of binding to CD16A, NKG2C, NKG2E, NKG2F, or NKG2H. In further embodiments, the polypeptide capable of binding to CD16A includes capable of binding to one or more of NKp46, 2B4, CD2, DNAM, NKG2C, NKG2D, NKG2E, NKG2F, or NKG2H. In some embodiments, the activation element is a polypeptide capable of binding to one or more of the following combinations: NKp46 and 2B4, NKp46 and CD2, NKp46 and DNAM, NKp46 and NKG2D, 2B4 and DNAM, or 2B4 and NKG2D. In some embodiments, the activation element can be two or more polypeptides capable of binding to polypeptides on the surface of a lymphocyte. In some embodiments, the activation element can be two or more polypeptides capable of binding to at least one of the following combinations: NKp46 and 2B4, NKp46 and CD2, NKp46 and DNAM, NKp46 and NKG2D, 2B4 and DNAM, or 2B4 and NKG2D.

In some embodiments, provided herein as separate aspects, or as components of, or as produced by, or used in, methods, uses, and compositions provided herein, are modified cells, in illustrative embodiments T cells, that have a dimmed surface polypeptide, or a population of any of the preceding modified cells that have a dimmed surface polypeptide. Such modified CD4+ cells or CD8+ cells or a population thereof can be CD3 dimmed, and can have the following characteristics (referred to herein as “Dimmed T Cell Characteristics”), and in illustrative embodiments have the following characteristics at the time of forming and/or administering the cell formulation:
i) at least 9%, 10% 15%, 20%, 30%, 40%, 50%, 60%, 70%, 80%, 90%, 95%, or 99%, or between 10% and 50%, 60%, 70%, 80%, 90%, 95%, or 99%, or between 50% and 60%, 70%, 80%, 90%, 95%, or 99%, or between 9%, 10% 15%, 20%, 30%, 40%, 50%, 60%, 70%, 80%, 90%, or 95% on the low end and 99% on the high end of the CD4+ cells in the cell formulation are surface CD3-;
ii) at least 18%, 20%, 30%, 40%, 50%, 60%, 70%, 80%, 90%, 95%, or 99%, or between 20% and 50%, 60%, 70%, 80%, 90%, 95%, or 99%, or between 50% and 60%, 70%, 80%, 90%, 95%, or 99%, or between 18%, 20%, 30%, 40%, 50%, 60%, 70%, 80%, 90%, or 95% on the low end and 99% on the high end of the CD8+ cells in the cell formulation are surface CD3-;
iii) within the population of cells that are either CD4+ or CD8+ in the cell formulation, at least 10.5%, 15%, 20%, 30%, 40%, 50%, 60%, 70%, 80%, 90%, 95%, or 99%, or between 10% and 50%, 60%, 70%, 80%, 90%, 95%, or 99%, or between 25% and 50%, 60%, 70%, 80%, 90%, 95%, or 99%, or between 50% and 60%, 70%, 80%, 90%, 95%, or 99%, or between 10.5%, 15%, 20%, 30%, 40%, 50%, 60%, 70%, 80%, 90%, or 95% on the low end and 99% on the high end, are surface CD3-;
iv) at least 1.5%, 2%, 3%, 4%, 5%, 6%, 7%, 8%, 9%, 10%, or 11%, or between 1.5% and 2%, 3%, 4%, 5%, 6%, 7%, 8%, 9%, 10%, or 11%, or between 5% and 6%, 7%, 8%, 9%, 10%, or 11%; or between 1.5%, 2%, 3%, 4%, 5%, 6%, 7%, 8%, 9% or 10% and 11% of the cells in the cell formulation, excluding RBCs, are surface CD3-CD4+;
v) at least 0.65%, 0.75%, 1 %, 1.5%, 2%, 2.5%, 3%, 3.5%, 4%, 4.5% or 5%; between 0.65% and 0.75%, 1%, 1.5%, 2%, 2.5%, 3%, 3.5%, 4%, 4.5% or 5%; between 1 % and 1.5%, 2%, 2.5%, 3%, 3.5%, 4%, 4.5% or 5%; or between 0.65%, 0.75%, 1%, 1.5%, 2%, 2.5%, 3%, 3.5%, 4%, or 4.5% on the low end and 5% on the high end of the cells in the cell formulation, excluding RBCs, are surface CD3-CD8+;
vi) less than 13%, 12%, 11%, 10%, 9%, 8%, 7%, 6%, 5%, 4%, 3%, 2%, 1%, 0.5%, or 0.1% of the cells in the cell formulation, excluding RBCs, are surface CD3+ and either CD4+ or CD8+ (the “Percent Total CD3+ cells”);
vii) within the population of cells that are either CD4+ or CD8+ in the cell formulation, less than 89%, 80%, 75%, 70%, 60%, 50%, 40%, 30%, 25%, 20%, 15%, 10%, 5%, or 1 %, or between 89% and 50%, 40%, 30%, 20%, 10%, 5%, or 1%, or between 75% and 50%, 40%, 30%, 20%, 10%, 5%, or 1%, or between 50% and 40%, 30%, 20%, 10%, 5%, or 1%, or between 1% and 5%, 10%, 20%, 30%, 40%, 50%, 60%, 70%, 75% or 89%, are surface CD3+;
viii) CD4+ and/or CD8+ cells in the cell formulation have lower surface expression of CD3 than the surface expression of CD3 on CD4+ and/or CD8+ cells in blood collected from a healthy subject or a population of healthy subjects, wherein the surface expression of CD3 in the cell formulation is lower by at least a 10%, 20%, 25%, 30%, 40%, 50%, 60%, 70%, 80%, 90%, 95% or 99%, or between 10% and 20%, 25%, 30%, 40%, 50%, 60%, 70%, 80%, 90%, 95% or 99%, or between 50% and 60%, 70%, 80%, 90%, 95% or 99%, or between 10%, 20%, 25%, 30%, 40%, 50%, 60%, 70%, 80%, 90% or 95% on the low end and 99% on the high end; and/or
ix) the Percent Total CD3+ cells is reduced by at least 19%, 20% 30%, 40%, 50%, 60%, 70%, 80%, 90%, or 95% after contacting polynucleotide vectors, and in illustrative embodiments RIPs, compared to the Percent Total CD3+ cells in the absence of contacting the polynucleotide vectors, and in illustrative embodiments, RIPs.

In some embodiments, provided herein as separate aspects, or as components of, or as produced by, or used in, methods, uses, and compositions provided herein, are modified cells, in illustrative embodiments modified NK cells, that have a dimmed surface polypeptide, or a population of modified cells that have a dimmed surface polypeptide. Such modified cells, for example modified NK cells, or a population thereof can have one or more of CD16A, NKp46, 2B4, CD2, DNAM, NKG2C, NKG2D, NKG2E, NKG2F, or NKG2H dimmed, and can have the following characteristics (referred to herein as “Dimmed NK Cell Characteristics”), and in illustrative embodiments have the following characteristics at the time of forming and/or administering a cell formulation:
i) at least 9%, 10% 15%, 20%, 30%, 40%, 50%, 60%, 70%, 80%, 90%, 95%, or 99%, or between 10% and 50%, 60%, 70%, 80%, 90%, 95%, or 99%, or between 50% and 60%, 70%, 80%, 90%, 95%, or 99%, or between 9%, 10% 15%, 20%, 30%, 40%, 50%, 60%, 70%, 80%, 90%, or 95% on the low end and 99% on the high end of the CD56+ cells in the cell formulation are surface CD16A-, NKp46-, 2B4-, CD2-, DNAM-, NKG2C-, NKG2D-, NKG2E-, NKG2F-, and/or NKG2H-;
ii) within the population of cells that are CD56+ in the cell formulation, at least 10.5%, 15%, 20%, 30%, 40%, 50%, 60%, 70%, 80%, 90%, 95%, or 99%, or between 10% and 50%, 60%, 70%, 80%, 90%, 95%, or 99%, or between 25% and 50%, 60%, 70%, 80%, 90%, 95%, or 99%, or between 50% and 60%, 70%, 80%, 90%, 95%, or 99%, or between 10.5%, 15%, 20%, 30%, 40%, 50%, 60%, 70%, 80%, 90%, or 95% on the low end and 99% on the high end, are surface CD16A-, NKp46-, 2B4-, CD2-, DNAM-, NKG2C-, NKG2D-, NKG2E-, NKG2F-, and/or NKG2H-;
iii) at least 1.5%, 2%, 3%, 4%, 5%, 6%, 7%, 8%, 9%, 10%, or 11%, or between 1.5% and 2%, 3%, 4%, 5%, 6%, 7%, 8%, 9%, 10%, or 11%, or between 5% and 6%, 7%, 8%, 9%, 10%, or 11%; or between 1.5%, 2%, 3%, 4%, 5%, 6%, 7%, 8%, 9% or 10% and 11% of the cells in the cell formulation, excluding RBCs, are CD56+ and surface CD16A-, NKp46-, 2B4-, CD2-, DNAM-, NKG2C-, NKG2D-, NKG2E-, NKG2F-, and/or NKG2H-;
iv) less than 13%, 12%, 11%, 10%, 9%, 8%, 7%, 6%, 5%, 4%, 3%, 2%, 1%, 0.5%, or 0.1% of the cells in the cell formulation, excluding RBCs, are surface CD16A+, NKp46+, 2B4+, CD2+, DNAM+, NKG2C+, NKG2D+, NKG2E+, NKG2F+, and/or NKG2H+ and CD56+ (the “Percent Total CD16A, NKp46, 2B4, CD2, DNAM, NKG2C, NKG2D, NKG2E, NKG2F, and/or NKG2H cells”);
v) within the population of cells that are CD56+ in the cell formulation, less than 89%, 80%, 75%, 70%, 60%, 50%, 40%, 30%, 25%, 20%, 15%, 10%, 5%, or 1 %, or between 89% and 50%, 40%, 30%, 20%, 10%, 5%, or 1%, or between 75% and 50%, 40%, 30%, 20%, 10%, 5%, or 1%, or between 50% and 40%, 30%, 20%, 10%, 5%, or 1 %, or between 1 % and 5%, 10%, 20%, 30%, 40%, 50%, 60%, 70%, 75% or 89%, are surface CD16A+, NKp46+, 2B4+, CD2+, DNAM+, NKG2C+, NKG2D+, NKG2E+, NKG2F+, and/or NKG2H+ and CD56+;
vi) CD56+ cells in the cell formulation have lower surface expression of CD16A, NKp46, 2B4, CD2, DNAM, NKG2C, NKG2D, NKG2E, NKG2F, and/or NKG2H than the surface expression of CD16A, NKp46, 2B4, CD2, DNAM, NKG2C, NKG2D, NKG2E, NKG2F, and/or NKG2H, respectively, on CD56+ cells in blood collected from a healthy subject or a population of healthy subjects, wherein the surface expression of CD16A, NKp46, 2B4, CD2, DNAM, NKG2C, NKG2D, NKG2E, NKG2F, and/or NKG2H in the cell formulation is lower by at least a 10%, 20%, 25%, 30%, 40%, 50%, 60%, 70%, 80%, 90%, 95% or 99%, or between 10% and 20%, 25%, 30%, 40%, 50%, 60%, 70%, 80%, 90%, 95% or 99%, or between 50% and 60%, 70%, 80%, 90%, 95% or 99%, or between 10%, 20%, 25%, 30%, 40%, 50%, 60%, 70%, 80%, 90% or 95% on the low end and 99% on the high end; and/or
vii) the Percent Total CD16A, NKp46, 2B4, CD2, DNAM, NKG2C, NKG2D, NKG2E, NKG2F, and/or NKG2H cells is reduced by at least 19%, 20% 30%, 40%, 50%, 60%, 70%, 80%, 90%, or 95% after contacting polynucleotide vectors, and in illustrative embodiments RIPS, compared to the Percent Total CD16A, NKp46, 2B4, CD2, DNAM, NKG2C, NKG2D, NKG2E, NKG2F, and/or NKG2H cells in the absence of contacting the polynucleotide vectors, and in illustrative embodiments, RIPs.

In any of the aspects herein, in illustrative embodiments that include the Dimmed T Cell Characteristics or the Dimmed NK Cell Characteristics, the modified cells can be recently activated within the prior 7, 6, 5, 4, 3, 2, or 1 days.

In some embodiments of any of the cell formulation aspects or embodiments herein, or any method or use aspect that includes a cell formulation, or any of the population embodiments, some of the modified CD4+, modified CD8+, modified CD56+, modified T cells and/or modified NK cells therein, are in cell aggregates. In some embodiments, at least 1%, 2%, 3%, 4%, 5%, 7.5%, 10%, 15%, 20%, or 25%, or between 1% and 10% 15%, 20%, 25%, 50%, and 75% of the white blood cells, modified CD4+ cells, modified CD8+ cells, modified CD56+ cells, modified T cells and/or modified NK cells in the cell formulation are in cell aggregates. In some embodiments, the cell aggregates are greater than 15, 25, 30, 35, 40, 50, or 60 µm in diameter, or between 25 and 50, 60, 75, 100, 125, 150, 200 or 250 µm in diameter. In some embodiments, modified cells are in aggregates comprising at least 4, 5, 6, 7, 8, 9, 10, 20, 30, 40, 50, 100, 250, 500, 1,000, 2,500, 5,000, or 10,000 white blood cells or 5 to 500, 5 to 250, 5 to 100, 10 to 500, 10 to 250, or 10 to 100 white blood cells. Furthermore, in some embodiments, including sub-embodiments of the immediately preceding embodiment, at least 1%, 2%, 3%, 4%, 5%, 7.5%, 10%, 15%, 20%, or 25%, or between 1% and 10% 15%, 20%, 25%, 50%, and 75% of white blood cells, the modified T and/or NK cells in the cell formulation are in aggregates comprising, or comprising at least 4, 5, 6, 8, or 5 to 500, 5 to 250, 5 to 100, 10 to 500, 10 to 250, or 10 to 100 white blood cells, modified T cells and/or NK cells. Further, in some embodiments, the cell formulation comprises aggregates of modified T cells and/or NK cells, in some embodiments along with unmodified T cells and/or NK cells and/or other white blood cells, capable of being retained by a coarse filter having a pore diameter of at least 15, 20, 25, 30, 40, 50, or 60 µm. In certain illustrative embodiments, at least 5% of the white blood cells, T cells, NK cells, modified T cells and/or modified NK cells are in cell aggregates. In certain sub-embodiments, the cell aggregates are greater than 40 µm in diameter and/or are capable of being retained by a course filter having a pore diameter of at least 40 µm. In some sub-embodiments, the cell aggregates comprise 5 to 500 white blood cells or modified T cells.

In some embodiments of any of the aspects or embodiments herein that include administering cells, populations, or cell formulations to a subject, a persisting population of genetically modified cells, and in illustrative embodiments genetically modified T cells and/or NK cells, or a population of progeny cells or genetically modified progeny cells derived from the modified cells, persists in the subject for at least 1, 2, 3, 4, 5, 6, 7, 14, 17, 21, or 28 days or 1, 2, or 1, 2, 3, 4, 5, 6, 7, 8, 9, 10, 11, or 12 months or 1, 2, 3, 4, or 5 years after administration. In some embodiments, at least 50%, 60%, 70%, 80%, 90% or 95% of the genetically modified cells, and in illustrative embodiments CAR-T cells, express a first polypeptide comprising the transgene, and in illustrative embodiments express the engineered T cell receptor, or the CAR. In some embodiments, at least 50%, 60%, 70%, 80%, 90% or 95% of the genetically modified cells expressing the first polypeptide comprising the transgene are circulating in the blood and/or at the site of a tumor, for example a solid tumor and the remainder of the genetically modified cells are subcutaneous. In some embodiments, the persisting population is subcutaneous, is circulating in the blood, and/or is at the site of a tumor, for example a solid tumor. In some embodiments, the subcutaneous region contains no artificial matrix components.

In some embodiments, the persisting population is detectable by histology. In some embodiments, the persisting population persists subcutaneously for at least or up to 14, 21, 28, 50, 60, 90 days and is detectable by histology. In some embodiments, the persisting population is detectable by FACS, for example FACs for the CAR or a removal tag (e.g. eTag), for example as 2 genetically modified cells/µl blood, or by qPCR, for example qPCR or sequencing of the transgene or across a chimeric junction of a CAR, or for a non-human subject treated with human engineered cells, such as human CAR-T cells, human RNAse P (hRNAseP). In some embodiments, the persisting population is detectable in the blood.

In some embodiments of any of the aspects provided herein, including but not limited to the method and use aspects provided hereinabove in this Exemplary Embodiments section, the modified cells, for example modified T cells and/or NK cells, or a population thereof, have a surface polypeptide dimmed, which in illustrative embodiments can be a TCR complex polypeptide, and in illustrative sub-embodiments, CD3. Such dimmed cells, including populations thereof, in illustrative embodiments exhibit any of the Dimmed T Cell Characteristics and/or Dimmed NK Cell Characteristics provided herein.

In some embodiments of any of the aspects provided herein, including but not limited to the method and use aspects provided hereinabove in this Exemplary Embodiments section, some (e.g. at least 5%, 7.5%, or 10%) of the modified cells, for example modified T cells and/or NK cells, or a population thereof, or a population thereof, are in aggregates, as disclosed herein.

In some embodiments of any of the aspects provided herein, including but not limited to the method and use aspects provided hereinabove in this Exemplary Embodiments section the cells form a population, which can be a persistent population, as disclosed herein.

In any of the persisting population or population of progeny cells aspects or embodiments herein, or any aspect or embodiment herein that includes a persisting population or a population of progeny cells, the number of modified cells, such as modified T cells and/or NK cells, and in illustrative embodiments genetically modified T cells and/or NK cells, comprises at least 100, 1 × 10³,1 × 10⁴, 1 × 10⁵, 1 × 10⁶, 1 × 10⁷,1 × 10⁸, 1 × 10⁹, 1 × 10¹⁰, 1 × 10¹¹, or 1 × 10¹² cells or between 1 × 10³ and 1 × 10⁴, 1 × 10⁵, 1 × 10°, 1 × 10⁷, 1 × 10⁸, or 1 × 10⁹ cells. In some embodiments, the modified cells, and in illustrative embodiments modified T cells and/or NK cells present in the cell formulation administered to a subject multiply at least 5, 10, 15, 20, 25, 50, 75, 100, 250, 500, 750, 1,000, 2,500, 5,000, or 10,000 fold in the subject, for example to form a persisting population or a population of progeny cells.

In some embodiments, the persisting population or the population of progeny cells express an engineered T cell receptor or a CAR, and the persisting population or the population of progeny cells is detected indirectly by a durable clinical response. For example, such persistence can be detected by detecting stable disease, a partial response, or a complete response with a duration of response of at least 3, 6, 9, 12, 18, or 24 months after initial observation of a clinical response, which in some embodiments is stable disease for patients experiencing progressive disease before administration of the cells, and in illustrative embodiments administration of the engineered T cell or CAR-T therapy.

In any of the aspects provided herein that include a step of collecting blood, the volume of blood collected can be for example, between 5 ml and 600 ml. More volumes and ranges are provided elsewhere in this specification, and in some embodiments, include the Small Volume Elements provided herein. In some embodiments when collected blood is processed using a filter, in illustrative embodiments a leukoreduction filter, the volume of blood sample applied to a filter is 600, 500, 400, 300, 200, 150, 120, 100, 75, 50, 40, 30, 25, 20, 15, 10, or 5 ml or less. In illustrative embodiments, the volume of blood sample applied to a filter is 50, 25, 20, 15, 10, 9, 8, 7, 6, 5, 4, 3, 2, or 1 ml or less.

In some embodiments of any of the aspects provided herein, the cell formulation, which in illustrative embodiments can be in a syringe, has a volume of between 0.5 ml and 20 ml, 15 ml, 10 ml, 5 ml, or 1 ml; or between 1 ml and 20 ml, 15 ml, 10 ml, 5 ml, 4 ml, 3 ml, 2 ml or less; or between 2 ml and 20 ml, 15 ml, 10 ml, 7 ml or 5 ml; or between 5 ml and 20 ml, 15 ml or 10 ml, or between 3 ml and 12 ml, or less than 3 ml. In some embodiments of any of the aspects or embodiments provided herein, wherein blood is collected from a subject, the blood collected has a volume of between 2.5 ml and 75 ml, 60 ml, 50 ml, 40 ml, 30 ml, 25 ml, 20 ml, 15 ml, 10 ml, or 5 ml, or between 5 ml and 75 ml, 60 ml, 50 ml, 40 ml, 30 ml, 25 ml, 20 ml, 15 ml, or 10 ml, or between 10 ml and 75 ml, 60 ml, 50 ml, 50 ml, 40 ml, 30 ml, 25 ml, or 20 ml, or between 15 ml and 75 ml, 60 ml, 50 ml, 50 ml, 40 ml, 30 ml, 25 ml, or 20 ml, or between 20 ml and 75 ml, 60 ml, 50 ml, 40 ml, 30 ml, or 25 ml, or between 25 ml and 75 ml, 70 ml, 60 ml, 50 ml, 40 ml, and 30 ml, or between 5 ml, 10 ml, or 15 ml on the low end and 20 ml on the high end. In some embodiments when collected blood is processed using a filter, in illustrative embodiments a leukoreduction filter, the volume of a whole blood sample, or a fraction thereof applied to a filter can be between 2.5 ml and 75, 50, 40, 30, 25, 20, 15, or 10. In illustrative embodiments, the volume of a whole blood sample, or a fraction thereof applied to a filter is between 10 ml and 50, 25, 20, 15 ml. In some embodiments, the volume of a whole blood sample, or fraction thereof applied to a filter is 10, 9, 8, 7, 6, 5, 4, 3, 2, or 1 ml or less. In some embodiments of any of the aspects or embodiments provided herein, the volume of the reaction mixture is between 2.5 ml and 75 ml, 60 ml, 50 ml, 40 ml, 30 ml, 25 ml, 20 ml, 15 ml, 10 ml, or 5 ml, or between 5 ml and 75 ml, 70 ml, 60 ml, 50 ml, 40 ml, 30 ml, 25 ml, 20 ml or 10ml, or between 10 ml and 75 ml, 70 ml, 60 ml, 50 ml, 40 ml, 30 ml, 25 ml, 20 ml, or 15 ml, or between 15 ml and 75 ml, 70 ml, 60 ml, 50 ml, 40 ml, 30 ml, 25 ml, or 20 ml, or between 20 ml and 75 ml, 70 ml, 60 ml, 50 ml, 40 ml, 30 ml, or 25 ml or between 25 ml and 75 ml, 70 ml, 60 ml, 50 ml, 40 ml, and 30 ml. The volumes for the cell formulation, the collected blood, and the reaction mixture in this paragraph are herein referred to as the “Small Volume Elements.” In illustrative subembodiments of embodiments that include the Small Volume Elements, the cell formulation is adapted for subcutaneous delivery, wherein the number of modified cells, such as modified T cells and/or NK cells in the modified cell formulation, is between 1.5 × 10⁴ and 1.5 × 10⁹, 1 × 10⁹, 1 × 10⁸, or 1 × 10⁷ or between 1 × 10⁵ and 1.5 × 10⁸, between 1 × 10⁵ and 1 × 10⁷, or between 1 × 10⁶ and 1 × 10⁸, or between 2 × 10⁶ and 1 × 10⁸, or in illustrative embodiments, between 3 × 10⁴ and 3 × 10⁷, between 1 × 10⁵ and 3 × 10⁷, or between 1 × 10⁶ and 3 × 10⁷ modified T cells, NK cells, CD4+ cells, CD8+ cells, and/or CD56+ cells.

In some embodiments, a contacting step is performed in a blood processing bag or other incubation bag, for example wherein whole blood, or a fraction thereof is added to an incubation bag comprising RIPs to form a reaction mixture or wherein the RIPs are added to the incubation bag comprising the whole blood to form the reaction mixture.

In illustrative embodiments of any of the encapsulated polynucleotide vectors (e.g. polynucleotide vector particles), and in illustrative embodiments retroviral particle, aspects provided herein, or any other aspect that includes a polynucleotide vector particle, the polynucleotide vector particle is substantially free of the protein transcript encoded by the nucleic acid of the polynucleotide vector particle, for example substantially free an engineered T cell receptor or CAR encoded by the nucleic acid of the polynucleotide vector polynucleotide vector particle.

In some embodiments, a sample such as a blood sample or a reaction mixture before, during, or after an incubation, is applied to a leukoreduction filter for example to remove RIPs not associated with lymphocytes. In some embodiments, at least 75%, 80%, 85%, 86%, 87%, 88%, 89%, 90%, 91%, 92%, 93%, 94%, 95%, 96%, 97%, 98%, 99 %, 99.5%, or 99.9% of the RIPs not associated with the lymphocytes are removed from a reaction mixture. In some embodiments, a reaction mixture is filtered over the leukoreduction filter at a flow rate of between 0.25 and 1, 1.5, 2, 2.5, 3, 3.5, 4, 4.5, or 5 ml/min, or between 0.5 and 2 ml/min. In some embodiments, the reaction mixture is filtered over the leukoreduction filter using a syringe. In some embodiments, the syringe is at less than an 80°, 75°, 70°, 65°, 60°, 55°, 50°. or 45° angle with respect to a channel (e.g. tubing) that is in fluid communication with the leukoreduction filter, when the reaction mixture is filtered. In some embodiments, blood cells and modified lymphocytes are not moved across a junction with a greater than 70°, 75°, or 80° angle during the removing the RIPs. In some embodiments, the leukoreduction filter has an effective filtration area of between 3 cm² and 5 cm² and in these or other embodiments, the diameter of the pore in the filter is between 2 and 6 µm. In some embodiments, cells retained by the leukoreduction filter after a reaction mixture is filtered over the leukoreduction filter, are washed with a volume of wash buffer that is 0.25 to 3 times the volume of the reaction mixture. In some embodiments, the removing of the RIPs is performed within a filter assembly comprising a syringe, the leukoreduction filter in fluid communication with the syringe, and one or more bags in fluid communication with the leukoreduction filter. In some embodiments, the removing of the RIPs is performed within a filter assembly comprising a second syringe and a second bag, wherein the second bag is in fluid communication with the leukoreduction filter.

In any aspect provided herein that includes a polynucleotide(s), such as an isolated polynucleotide(s) encoding an anti-idiotype polypeptide, and one or more of a CAR, a cytokine, one or more inhibitory RNA, and/or an LE, such polynucleotides or isolated polynucleotides can be contained in one or more containers, and for example in 0.1 ml to 10 ml of a solution. Such polynucleotides can contain substantially-pure, GMP grade, recombinant vectors (e.g., replication incompetent retroviral particles). In some embodiments, such polynucleotides comprise recombinant naked DNA vectors. In illustrative embodiments, such polynucleotides are a container of replication incompetent retroviral particles having between 1 × 10⁶ and 5 × 10⁹, 1 × 10⁷ and 1 × 10⁹, 5 × 10⁶ and 1 × 10⁸, 1 × 10⁶ and 5 × 10⁷, 1 × 10⁶ and 5 × 10⁶ or between 5 ×10⁷ and 1 ×10⁸ retroviral Transducing Units (TUs) or TUs/ml, or at least 100, 1,000, 2,000 or 2,500 TUs/ng p24.

In any of the aspects provided herein, the contacting step including with an optional incubation combined can be performed (or can occur) for 14, 12, or 10 hours or less, or in illustrative embodiments for 8, 6, 4, 3, 2, or 1 hour or less, or in certain further illustrative embodiments less than 8 hours, less than 6 hours, less than 4 hours, 2 hours, less than 1 hour, less than 30 minutes or less than 15 minutes, but in each case there is at least an initial contacting step in which retroviral particles and cells are brought into contact in suspension in a transduction reaction mixture. In other embodiments, the reaction mixture can be incubated for between 15 minutes and 12 hours, 15 minutes and 10 hours, 15 minutes and 8 hours, 15 minutes and 6 hours, 15 minutes and 4 hours, 15 minutes and 2 hours, 15 minutes and 1 hour, 15 minutes and 45 minutes, or 15 minutes and 30 minutes. In other embodiments, the reaction mixture can be incubated for between 30 minutes and 12 hours, 30 minutes and 10 hours, 30 minutes and 8 hours, 30 minutes and 6 hours, 30 minutes and 4 hours, 30 minutes and 2 hours, 30 minutes and 1 hour, or 30 minutes and 45 minutes. In other embodiments, the reaction mixture can be incubated for between 1 hour and 12 hours, 1 hour and 8 hours, 1 hour and 4 hours, or 1 hour and 2 hours. In another illustrative embodiment, the contacting is performed for between an initial contacting step only (without any further incubating in the reaction mixture including the retroviral particles free in suspension and cells in suspension) without any further incubation in the reaction mixture, or a 5 minute, 10 minute, 15 minute, 30 minute, or 1 hour incubation in the reaction mixture. In certain embodiments, the contacting can be performed (or can occur) for between 30 seconds or 1, 2, 5, 10, 15, 30 or 45 minutes, or 1, 2, 3, 4, 5, 6, 7, or 8 hours on the low end of the range, and between 10 minutes, 15 minutes, 30 minutes, or 1, 2, 4, 6, 8, 10, 12, 18, 24, 36, 48, and 72 hours on the high end of the range. In illustrative embodiments, the contacting can be performed (or can occur) for between a contacting only, 30 seconds or 1, 2, 5, 10, 15, 30 or 45 minutes, or 1 hour on the low end of the range, and between 2, 4, 6, and 8 hours on the high end of the range. In some embodiments, the RIPs can be immediately washed out after adding them to the cell(s) to be modified, genetically modified, and/or transduced such that the contacting time is carried out for the length of time it takes to wash out the RIPs. Accordingly, typically the contacting includes at least an in initial contacting step in which a retroviral particle(s) and a cell(s) are brought into contact in suspension in a transduction reaction mixture. Such methods can be performed without prior activation.

In illustrative embodiments of methods provided herein, the contacting step with optional incubating, is performed at temperatures between 32° C. and 42° C., such as at 37° C. as provided in more detail herein. In other illustrative embodiments, the contacting step with optional incubating, is performed at temperatures lower than 37° C., such as between 1° C. and 25° C., 2° C. and 20° C., 2° C. and 15° C., 2° C. and 6° C., or 3° C. and 6° C. The optional incubating associated with the contacting step at these temperatures can be performed for any length of time discussed herein. In illustrative embodiments, the optional incubating associated with these temperatures is performed for 1 hour or less, such as for 0 to 55 minutes (i.e., 55 minutes or less), 0 to 45 minutes (i.e., 45 minutes or less), 0 to 30 min (i.e., 30 minutes or less), 0 to 15 minutes (i.e., 15 minutes or less), 0 to 10 minutes (i.e., 10 minutes or less), 0 to 5 minutes (i.e., 5 minutes or less), 5 to 30 minutes, 5 to 15 minutes, or 10 to 30 minutes. In further illustrative embodiments, the cold contacting and incubating is performed at temperatures between 2° C. and 15° C. for between 0 to 55 minutes, 0 to 45 minutes, 0 to 30 min, 0 to 15 minutes, 0 to 10 minutes, 0 to 5 minutes, 5 to 15 minutes, or 10 to 30 minutes. In other further illustrative embodiments, the cold contacting and incubating is performed for 5 to 30 minutes at a temperature between 1° C. and 25° C., 2° C. and 20° C., 2° C. and 15° C., 2° C. and 6° C., or 3° C. and 6° C.

In certain embodiments that comprise a contacting step at the colder temperatures provided immediately above, a secondary incubation is typically performed by suspending cells after an optional wash step in a solution comprising recombinant vectors, in illustrative embodiments retroviral particles. In illustrative embodiments, the secondary incubation is performed at temperatures between 32° C. and 42° C., such as at 37° C. The optional secondary incubation can be performed for any length of time discussed herein. In illustrative embodiments, the optional secondary incubation is performed for 6 hours or less, such as for 1 to 6 hours, 1 to 5 hours, 1 to 4 hours, 1 to 3 hours, 1 to 2 hours, 2 to 4 hours, 30 minutes to 4 hours, 10 minutes to 4 hours, 5 minutes to 4 hours, 5 minutes to 1 hour, 1 minute to 5 minutes, or less than 5 minutes. Thus, in some illustrative embodiments, optionally the T cell and/or NK cell activation element is on the surface of the RIPs, the contacting is performed at between 2° C. and 15° C., and optionally between 2° C. and 6° C., for less than 1 hour, optionally after which the TNCs are incubated at between 32° C. and 42° C. for between 5 minutes and 8 hours, or in illustrative embodiments, between 5 minutes and 4 hours, and optionally after which the modified T cells and/or NK cells are collected on a filter to form the cell formulation

In some embodiments, no more than 16 hours, 14 hours, 12 hours, 8 hours, 4 hours, 2 hours, or 1 hour pass, or between 5, 10, 15, 30, 45, or 60 minutes on the low end of the range, and between 1.5, 2, 4, 6, 8, 10, 12, 14, and 16 hours on the high end of the range, for example between 5 minutes and 16 hours, 5 minutes and 12 hours, 5 minutes and 8 hours, 5 minutes and 6 hours, 5 minutes and 4 hours, 5 minutes and 3 hours, 5 minutes and 2 hours, or 5 minutes and 1 hour pass, between the time blood, TNCs, or PBMCs are contacted with recombinant nucleic acid vectors, which in illustrative embodiments are replication incompetent retroviral particles, and the time the modified cells are suspended and thus formulated in a delivery solution to form a cell formulation. In some embodiments, the time between when the cells are contacted with the replication incompetent retroviral particles and when the modified cells are formulated in a delivery solution can be between 1 and 16 hours, 1 and 14 hours, 1 and 12 hours, 1 and 8 hours, 1 and 6 hours, 1 and 4 hours, or 1 and 2 hours. In some embodiments, no more than 16 hours, 14 hours, 12 hours, 8 hours, 4 hours, 2 hours, or 1 hours pass between the time blood is collected from the subject and the time the modified lymphocytes are reintroduced into the subject. In some embodiments, the time between when the blood is collected from the subject and when the modified lymphocytes are reintroduced into the subject can be between 1 and 16 hours, 1 and 14 hours, 1 and 12 hours, 1 and 8 hours, 1 and 6 hours, 1 and 4 hours, or 1 and 2 hours.

In any of the aspects provided herein that include an administering step, in illustrative embodiments, administered cells are processed ex vivo, for example using any of the methods comprising contacting and formulating steps provided herein, for less than 24, 18, 12, 10, 8, 6, 4, 2, or 1 hour or 30 or 15 minutes, or for between 15 minutes and 24, 18, 12, 10, 8, 6, 4, 2, 1, or 0.5 hours, or for between 1 hour and 24, 18, 12, 10, 8, 6, 4, or 2 hours, before the administration. Thus, in certain embodiments such ex vivo times can be the time between collecting blood from a subject and intravenous, intramuscular, intratumor, intraperitoneal, and in illustrative embodiments subcutaneous administration of modified lymphocytes, in illustrative embodiments derived from lymphocytes from the subject, to the subject

In some embodiments of any relevant aspect herein, some or all of the T and NK cells do not yet express the recombinant nucleic acid or have not yet integrated the recombinant nucleic acid into the genome of the cell before being used or included in any of the methods or compositions provided herein, including, but not limited to, being introduced or reintroduced back into a subject, or before, or at the time of being used to prepare a cell formulation. In some embodiments, at least 25%, 30%, 40%, 45%, 50%, 55%, 60%, 65%, 70%, 75%, 80%, 85%, 90%, 95%, 96%, 97%, 98%, 99%, or all of the modified T and NK cells do not express a CAR or transposase, and/or do not have a CAR associated with their cell membrane, when the modified lymphocytes are introduced or reintroduced back into a subject, and in illustrative embodiments introduced or reintroduced back into a subject subcutaneously or intramuscularly, or when used to prepare a cell formulation. In other embodiments, provided herein are cell formulations wherein at least 25%, 30%, 40%, 45%, 50%, 55%, 60%, 65%, 70%, 75%, 80%, 85%, 90%, 95%, 96%, 97%, 98%, 99%, or all of the modified T and/or NK cells in a cell formulation contain recombinant viral reverse transcriptase and/or integrase. In illustrative embodiments, at least 25%, 50%, 60%, 70%, 75%, 80%, 85%, 90%, 95%, 96%, 97%, 98%, 99%, or all of the modified T and NK cells do not express a CAR, and/or do not have a CAR associated with their cell membrane when the modified lymphocytes are introduced or reintroduced back into a subject, and in illustrative embodiments introduced or reintroduced back into a subject subcutaneously or intramuscularly, or when used to prepare a cell formulation. In illustrative embodiments, at least 25%, 50%, 55%, 60%, 65%, 70%, 75%, 80%, 85%, 90%, 95%, 96%, 97%, 98%, 99%, or all of the modified T and NK cells do not express a recombinant mRNA (e.g., encoding a CAR) when the lymphocytes are introduced or reintroduced into a subject, and in illustrative embodiments introduced or reintroduced back into a subject subcutaneously or intramuscularly, or when used to prepare a cell formulation. In some embodiments, greater than 50%, 60%, 70%, 75%, 80% or 90% of the cells, NK cells, and/or T cells in a cell formulation are viable.

In some embodiments, at least 20%, 25%, 30%, 35%, 40%, 45%, 50%, 55%, 60%, 65%, 70%, 75%, 80%, 85%, 90%, 95%, 96%, 97%, 98%, 99%, or all of the modified T and NK cells do not have the recombinant nucleic acid stably integrated into their genomes when the lymphocytes are introduced or reintroduced into a subject, and in illustrative embodiments introduced or reintroduced back into a subject subcutaneously or intramuscularly, or when used to prepare a cell formulation. In illustrative embodiments, at least 25%, 30%, 40%, 45%, 50%, 55%, 60%, 65%, 70%, 75%, 80%, 85%, 90%, 95%, 96%, 97%, 98%, 99%, or all of the modified T and NK cells do not have the recombinant nucleic acid stably integrated into their genomes when the lymphocytes are introduced or reintroduced into a subject, and in illustrative embodiments introduced or reintroduced back into a subject subcutaneously or intramuscularly, or when used to prepare a cell formulation. In some embodiments of any of the aspects herein that include modified, genetically modified, transduced, and/or stably transfected lymphocytes, any percentage of the lymphocytes can be modified, genetically modified, transduced, and/or stably transfected when the lymphocytes are introduced or reintroduced back into a subject, and in illustrative embodiments introduced or reintroduced back into a subject subcutaneously or intramuscularly, or when a cell formulation is prepared. In some embodiments, at least 4%, 5%, 10%, 15%, 20%, 25%, 30%, 35%, 40%, 45%, 50%, 55%, 60%, 65%, 70%, 75%, 80%, 85%, 90%, or 95% of the lymphocytes are modified. In illustrative embodiments, between 4%, 5%, 10%, 15%, 20%, 25%, 30%, 35%, 40%, 45%, 50%, 55%, 60%, 65%, and 70% of the lymphocytes are modified on the low end of the range and 10%, 15%, 20%, 25%, 30%, 35%, 40%, 45%, 50%, 55%, 60%, 65%, 70%, 75%, 80%, 85%, 90%, and 95% of the lymphocytes are modified on the high end of the range. In some embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the modified lymphocytes are not genetically modified, transduced, or stably transfected. In illustrative embodiments, at least 25%, 30%, 35%, 40%, 45%, 50%, 55%, 60%, 65%, 70%, 75%, 80%, 85%, 90%, 95%, 96%, 97%, 98%, 99%, or all of the modified lymphocytes are not genetically modified, transduced, or stably transfected. In some embodiments, between 5%, 10%, 15%, 20%, 25%, 30%, 35%, 40%, 45%, 50%, 55%, 60%, 65%, and 70% of the modified lymphocytes are not genetically modified, transduced, or stably transfected on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified lymphocytes are not genetically modified, transduced, or stably transfected on the high end of the range (e.g., between 10% and 95%). Genetically modified lymphocytes containing a recombinant nucleic acid can either have the recombinant nucleic acid extrachromosomal or integrated into the genome. In some embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the genetically modified lymphocytes have an extrachromosomal recombinant nucleic acid. In illustrative embodiments, at least 25%, 30%, 35%, 40%, 45%, 50%, 55%, 60%, 65%, 70%, 75%, 80%, 85%, 90%, 95%, 96%, 97%, 98%, 99%, or all of the genetically modified lymphocytes have an extrachromosomal recombinant nucleic acid. In some embodiments, between 5%, 10%, 15%, 20%, 25%, 30%, 35%, 40%, 45%, 50%, 55%, 60%, 65%, and 70% of the modified or genetically modified lymphocytes have an extrachromosomal recombinant nucleic acid on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified or genetically modified lymphocytes have an extrachromosomal recombinant nucleic acid on the high end of the range (e.g. between 10% and 95%). In some embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the modified or genetically modified lymphocytes are not transduced or stably transfected. In illustrative embodiments, at least 25%, 30%, 35%, 40%, 45%, 50%, 55%, 60%, 65%, 70%, 75%, 80%, 85%, 90%, 95%, 96%, 97%, 98%, 99%, or all of the modified or genetically modified lymphocytes are not transduced or stably transfected. In some embodiments, between 5%, 10%, 15%, 20%, 25%, 30%, 35%, 40%, 45%, 50%, 55%, 60%, 65%, and 70% of the modified or genetically modified lymphocytes are transduced or stably transfected on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified or genetically modified lymphocytes are not transduced or stably transfected on the high end of the range.

In some embodiments of any aspect herein wherein a formulation is administered to a subject, a second formulation is administered to the subject at a second, third, fourth, etc. timepoint between 1 day and 1 month, 2 months, 3 months, 6 months, or 12 months after the administering a first cell formulation, wherein the second formulation can be identical to the first formulation, or can comprises any of the formulations provided herein. i) a cytokine, ii) an antibody, antibody mimetic, or polypeptide that is capable of binding CD3, CD28, OX40, 4-1BB, ICOS, CD9, CD53, CD63, CD81, and/or CD82, and/or iii) a source of the cognate antigen recognized by the CAR, and optionally wherein the cytokine is IL-2, IL-7, IL-15, or IL-21, or a modified version of any of these cytokines that is capable of binding to and activating a native receptor for the cytokine.

In some embodiments of any of the aspects herein that include a cell mixture or cell formulation, any cell in a cell mixture can be enriched. For example, a cell useful in adoptive cell therapy, such as one or more cell populations of T and/or NK cells, can be enriched prior to formulation for delivery. In some embodiments, the one or more cell populations can be enriched after the cell mixture is contacted with a recombinant nucleic acid vector, such as a replication incompetent retroviral particle. In some embodiments, enriching the one or more cell populations can be performed at the same time as any of the methods of genetic modification disclosed herein, and in illustrative embodiments genetic modification with a replication incompetent retroviral particle.

In some embodiments of any of the aspects herein that include a cell mixture or cell formulation, one or more unwanted cell populations can be depleted, such that the desired cells in the cell mixture or cell formulation are enriched. In some embodiments, the one or more cell populations can be depleted by negative selection prior to being contacted with a recombinant nucleic acid vector, such as a replication incompetent retroviral particle. In some embodiments, the one or more cell populations can be depleted by negative selection after the cell mixture is contacted with a recombinant nucleic acid vector, such as a replication incompetent retroviral particle. In some embodiments, depleting the one or more cell populations can be performed before or at the same time as any of the methods of genetic modification disclosed herein, and in illustrative embodiments genetic modification with a replication incompetent retroviral particle.

In some embodiments, the unwanted cells include cancer cells. Cancer cells from many types of cancer can enter the blood and could be unintentionally genetically modified at a low frequency along with the lymphocytes using the methods provided herein. In some embodiments, the cancer cell can be derived from any cancer, including, but not limited to: renal cell carcinoma, gastric cancer, sarcoma, breast cancer, lymphoma, B cell lymphoma, a B cell lymphoma such as diffuse large B cell lymphoma (DLBCL), Hodgkin’s lymphoma, non-Hodgkin’s B-cell lymphoma (B-NHL), neuroblastoma, glioma, glioblastoma, medulloblastoma, colorectal cancer, ovarian cancer, prostate cancer, mesothelioma, lung cancer (e.g., small cell lung cancer), melanoma, leukemia, chronic lymphocytic leukemia (CLL), acute lymphocytic leukemia (ALL), acute myelogenous leukemia (AML), or chronic myelogenous leukemia (CML), or any of the cancers listed in this disclosure. In illustrative embodiments, the CAR-cancer cell can be derived from a lymphoma, and, in illustrative embodiments, a B-cell lymphoma.

In certain embodiments of any of the aspects herein that include blood cells, the blood cells in the reaction mixture comprise at least 10% neutrophils and at least 0.5% eosinophils, as a percent of the white blood cells in the reaction mixture.

In certain embodiments of any of the aspects herein that include a reaction mixture and/or a cell formulation, the reaction mixture and/or the cell formulation comprises at least 5%, 10%, 20%, 25%, 30%, or 40% neutrophils as a percent of cells in the reaction mixture or cell formulation, or between 20% and 80%, 25% and 75%, or 40% and 60% neutrophils as a percent of white blood cells in the reaction mixture or cell formulation.

In certain embodiments of any of the aspects herein that include a reaction mixture and/or a cell formulation, the reaction mixture and/or the cell formulation comprises at least 0.1% eosinophils, or between 0.25% and 8% eosinophils, or between 0.5% and 4% as a percent of white blood cells in the reaction mixture or cell formulation.

In certain embodiments of any of the aspects herein that include blood cells, the blood cells in the reaction mixture are not subjected to a PBMC enrichment procedure before the contacting.

In certain embodiments of any of the aspects herein that include a reaction mixture, the reaction mixture is formed by adding the recombinant retroviral particles to whole blood.

In certain embodiments of any of the aspects herein that include a reaction mixture, the reaction mixture is formed by adding the recombinant retroviral particles to substantially whole blood comprising an effective amount of an anticoagulant.

In certain embodiments of any of the aspects herein that include a reaction mixture, the reaction mixture comprises an anticoagulant. For example, in certain embodiments, the anticoagulant is selected from the group consisting of acid citrate dextrose, EDTA, or heparin. In certain embodiments, the anticoagulant is other than acid citrate dextrose. In certain embodiments, the anticoagulant comprises an effective amount of heparin.

In certain embodiments of any of the aspects herein that include a reaction mixture, the reaction mixture is in a blood bag during the contacting.

In certain embodiments of any of the aspects herein that are or include a method, the method further comprises administering the modified T cells and/or NK cells to a subject subcutaneously. Optionally in such certain embodiments, the modified T cells and/or NK cells are delivered in a cell formulation that further comprises neutrophils. Furthermore, optionally in such certain embodiments, the neutrophils are present in the cell formulation at a concentration too high for safe intravenous delivery, and/or the cell formulation comprises 5%, 10%, 15%, 20%, or 25% neutrophils. In some embodiments of any of the methods herein that include a collecting, contacting, and an administrating step, the modified lymphocytes are introduced back into the subject within 14 hours, 12 hours, 8 hours, 6 hours, 4 hours, 2 hours, 1 hour, or 30 minutes from the time the blood comprising the lymphocytes is collected from the subject. In illustrative embodiments, such methods include subcutaneous administration. In illustrative embodiments, such methods include collecting blood cells using apheresis, or filtration of blood cells or modified lymphocytes over a filter, such as a leukoreduction filter.

In certain embodiments of any of the aspects herein that includes a method, the method further comprises administering the modified T cells and/or NK cells to the subject subcutaneously in the presence of a hyaluronidase. In further illustrative subembodiments, the T cells and/or NK cells that were modified, were obtained from the subject.

In further subembodiments of these embodiments including administering the modified and in illustrative embodiments genetically modified T cells and/or NK cells to the subject subcutaneously in the presence of a hyaluronidase, the modified T cells and/or NK cells are delivered subcutaneously to a subject in a volume between 1 ml and 5 ml. In further subembodiments, the T cells and/or NK cells are in blood drawn from a subject, and the modified T cells and/or NK cells are delivered back into the subject, and in further embodiments within 1-14, 1-8 hours, 1-6 hours, 1-4 hours, 1-2 hours, or within 1 hour from the time the blood is drawn from the subject.

In certain embodiments of any of the aspects herein that include a reaction mixture, the reaction mixture is in contact with a leukoreduction filter assembly in a closed cell processing system before the contacting, at the time the recombinant retroviral particles and the blood cells are contacted, during the contacting comprising an optional incubating in the reaction mixture, and/or after the contacting comprising the optional incubating in the reaction mixture.

In some embodiments of any of the aspects herein, at least 10%, 20%, 25%, 30%, 40%, 50%, most, 60%, 70%, 75%, 80%, 90%, 95%, or 99% of the T cells are resting T cells, or of the NK cells are resting NK cells, when they are combined with the replication incompetent retroviral particles to form the reaction mixture.

In any of the aspects herein that include modifying cells, the cell or cells are not subjected to a spinoculation procedure, for example not subjected to a spinoculation of at least 800 g for at least 30 minutes.

In some embodiments of any of the aspects herein that include a method, the method further comprises administering the modified T cells and/or NK cells to a subject, optionally wherein the subject is the source of the blood cells. In some subembodiments of these and embodiments of any of the methods and uses herein, including those in this Exemplary Embodiments section, provided that it is not incompatible with, or already stated, the modified, genetically modified, and/or transduced lymphocyte (e.g. T cell and/or NK cell) or population thereof, undergoes 4 or fewer cell divisions ex vivo prior to being introduced or reintroduced into the subject. In some embodiments, no more than 8 hours, 6 hours, 4 hours, 2 hours, or 1 hour pass(es) between the time blood is collected from the subject and the time the modified lymphocytes are reintroduced into the subject. In some embodiments, all steps after the blood is collected and before the blood is reintroduced, are performed in a closed system, optionally in which a person monitors the closed system throughout the processing. In some embodiments, at least 10%, 15%, 20%, 25%, 30%, 35%, 40%, 45%, 50%, 55%, 60%, 65%, 70%, 75%, 80%, 85%, or 90% of the modified lymphocytes in the solution can include a pseudotyping element or a T cell activating antibody on their surfaces. In some embodiments, the pseudotyping element and/or a T cell activating antibody can be bound to the surface of the modified lymphocytes through, for example, a T cell receptor, and/or the pseudotyping element and/or a T cell activating antibody can be present in the plasma membrane of the modified lymphocytes.

In any of the kits provided hereinabove, the first and/or second polynucleotides can comprise any self-driving CAR provided herein. Additional kit aspects and embodiments are provided hereinbelow, and in the Detailed Description herein, outside this Exemplary Embodiments section.

For any of the aspects provided herein that include a syringe, in illustrative embodiments, the syringe is compatible with, effective for, and/or adapted for intramuscular, and in illustrative embodiments subcutaneous delivery, and/or is effective to inject intramuscularly, effective to inject subcutaneously, adapted to inject intramuscularly, and/or adapted to inject subcutaneously. For example, the syringe can have a needle with a gauge between 20 and 22 and a length between 1 inch and 1.5 inches for intramuscular delivery and a needle with a gauge between 26 and 30 and a length between 0.5 inches and 0.625 inches for subcutaneous delivery.

In certain embodiments of any of the aspects and other embodiments herein that comprise a polynucleotide that encodes a CAR and an LE (e.g., polynucleotides, RIPs, cell formulations, populations, genetically modified lymphocytes, reaction mixtures, mammalian packaging cell lines comprising a packageable RNA genome for a replication incompetent retroviral particle, kits, uses of a RIP(s) in the manufacture of a kit for genetically modifying and/or transducing a lymphocyte, methods for genetically modifying and/or transducing a T cell or an NK cell, methods for administering a genetically modified lymphocyte to a subject), the polynucleotide can include or encode an ant-idiotype polypeptide and one or more of a CAR, and LE, a cytokine, and one or more inhibitory RNA molecules.

In some embodiments, the self-driving CAR embodiment can be a polynucleotide comprising a first transcriptional unit operably linked to an inducible promoter inducible in at least one of a T cell or an NK cell, and a second transcriptional unit operably linked to a constitutive T cell or NK cell promoter, wherein the first transcriptional unit and the second transcriptional units are arranged divergently, wherein the first transcriptional unit encodes an LE, and wherein the second transcriptional unit encodes an anti-idiotype polypeptide and a CAR, wherein the CAR comprises an ASTR, a transmembrane domain, and an intracellular activating domain.

In any of the aspects herein that include recombinant retroviral particles in a container and/or reaction mixture, the recombinant retroviral particles are present in the container and/or reaction mixture at an MOI of between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15 or at least 0.1, 0.5, 1, 2, 2.5, 3, 5, 10 or 15 or are present in the reaction mixture at an MOI of at least 0.1, 0.5, 1, 2, 2.5, 3, 5, 10 or 15. For kit and isolated retroviral particle embodiments, such MOI can based on 1, 2.5, 5, 10, 20, 25, 50, 100, 250, 500, or 1,000 ml assuming 1×10⁶ target cells/ml, for example in the case of whole blood, assuming 1×10⁶ PBMCs/ml of blood.

In any of the aspects herein that include a contacting cells with retroviral particles, sufficient retroviral particles are present in a reaction to achieve an MOI of between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15 or at least 0.1, 0.5, 1, 2, 2.5, 3, 5, 10 or 15, or to achieve an MOI of at least 0.1, 0.5, 1, 2, 2.5, 3, 5, 10 or 15.

In any of the aspects herein that include a genetically modified T cell and/or NK cell, at least 5%, at least 10%, at least 15%, or at least 20% of the T cells and/or NK cells are genetically modified, or between 5% and 85%, or between 5% and 20%, 25%, 50%, 60%, 70%, 80%, or 85%, or between 5%, 10%, 15%, 20%, or 25% on the low end of the range, and 20%, 25%, 50%, 60%, 70%, 80%, or 85% on the high end of the range.

In any of the aspects herein that include, RIPs, the RIPs are lentiviral particles. In further illustrative embodiments, the modified cell is a modified T cell or a modified NKT cell.

In any of the aspects herein that include a polynucleotide including one or more transcriptional units, the one or more transcriptional units can encode an anti-idiotype polypeptide and a polypeptide comprising a CAR. In some embodiments, the CAR is a microenvironment restricted biologic (MRB)-CAR. In other embodiments, the ASTR of the CAR binds to a tumor associated antigen. In other embodiments, the ASTR of the CAR is a microenvironment-restricted biologic (MRB)-ASTR.

In certain embodiments, any of the aspects and embodiments provided herein that include a polynucleotide that comprises a nucleic acid sequences operatively linked to a promoter active in T cells and/or NK cells, the polynucleotide encodes at least one polypeptide lymphoproliferative element comprising an anti-idiotype extracellular recognition domain. In illustrative embodiments, the anti-idiotype extracellular recognition domain is capable of binding the idiotype of an antibody that does not induce cytotoxicity. In illustrative embodiments, the polypeptide lymphoproliferative element is any of the polypeptide lymphoproliferative elements disclosed herein. In some embodiments, any or all of the nucleic acid sequences provided herein can be operably linked to a riboswitch. In some embodiments, the riboswitch is capable of binding a nucleoside analog. In some embodiments, the nucleoside analog is an antiviral drug.

In certain illustrative embodiments of any of the aspects herein that include blood cells in a reaction mixture, the blood cells in the reaction mixture are blood cells that were produced by a PBMC enrichment procedure and comprise PBMCs, or the blood cells in illustrative embodiments are PBMCs. In illustrative embodiments, such embodiments including PMBC enrichment are not combined with an embodiment where the reaction mixture includes at least 10% whole blood. Thus, in certain illustrative embodiments herein, the blood cells in a reaction mixture are the PBMC cell fraction from a PBMC enrichment procedure to which retroviral particles are added to form the reaction mixture, and in other illustrative embodiments, the blood cells in a reaction mixture are from whole blood to which retroviral particles are added to form the reaction mixture.

In any of the aspects and embodiments provided herein that include, or optionally include, a nucleic acid sequence encoding an inhibitory RNA molecule, the inhibitory RNA molecule targets any of the gene (e.g. mRNAs encoding) targets identified for example in the Inhibitory RNA Molecules section herein.

In illustrative embodiments of any of the kits, delivery solutions and/or cell formulations provided herein, especially those that effective for, or adapted for intramuscular and in illustrative embodiments subcutaneous delivery, the delivery solution and/or cell formulation is a depot formulation, or the cell formulation is an emulsion of cells that promotes cell aggregation. In some embodiments, a depot delivery solution comprises an effective amount of alginate, hydrogel, PLGA, a cross-linked and/or polymer hyaluronan, PEG, collagen, and/or dextran to form a depot formulation. In some embodiments the delivery solution and/or cell formulation is designed for controlled or delayed release. In some embodiments, the delivery solution and/or cell formulation includes components that form an artificial extracellular matrix such as a hydrogel.

In any of the aspects and embodiments provided herein that include, or optionally include, a cell mixture, delivery solution, or cell formulation, the cell mixture, delivery solution, or cell formulation can have a pH and ionic composition that provides an environment in which cells can survive, for example for at least 1 hour, and typically can survive for at least 4 hours. In some embodiments, the pH can be between pH 6.5 to pH 8.0, pH 7.0 to pH 8.0, or pH 7.2 to pH 7.6. In some embodiments, for example, when the RIP has a polynucleotide that encodes an MRB-CAR, the pH can be between pH 6.0 to pH 7.0, for example, pH 6.2 to pH 7.0, or pH 6.4 to pH 7.0, or pH 6.4 to pH 6.8. In some embodiments, the cell mixture, delivery solution, or cell formulation can be maintained by a buffer such as a phosphate buffer or bicarbonate present at a concentration effective for maintaining pH in a target range. In some embodiments, a cell mixture, delivery solution, or cell formulation can include a saline composition with salts, for example 0.8 to 1.0 or about 0.9 or 0.9 percent salts such as sodium chloride. In some embodiments, the delivery solution is or includes PBS. In some embodiments of a delivery solution and resulting cell formulation herein, the concentration of Na⁺ is between 110 mM and 204 mM, the concentration of Cl- is between 98 mM and 122 mM, and/or the concentration of K⁺ is between 3 mM and 6 mM.

In some embodiments of any of the aspects herein that include a modified or genetically modified T cell or NK cell, or methods, compositions, and kits for genetically modifying T cells and/or NK cells, the proliferation and survival of genetically modified T cells and/or NK cells expressing a CAR can be induced by cross-linking CAR molecule within a genetically modified T cell or NK cell, in the absence of the CAR molecules binding to their cognate antigens. Thus, in some embodiments, a T cell or NK cell can comprise an epitope tag bound by an antibody and cross-linked to an epitope tag of a second CAR on the same T cell or NK cell. In some embodiments, the extracellular domain of the CAR can include the epitope tag. In illustrative embodiments, the epitope tag can be in the stalk domain. In some embodiments, the epitope tag can be His5 (HHHHH; SEQ ID NO:76), HisX6 (HHHHHH; SEQ ID NO:77), c-myc (EQKLISEEDL; SEQ ID NO:75), Flag (DYKDDDDK; SEQ ID NO:74), Strep Tag (WSHPQFEK; SEQ ID NO:78), HA Tag (YPYDVPDYA; SEQ ID NO:73), RYIRS (SEQ ID NO:79), Phe-His-His-Thr (SEQ ID NO:80), or WEAAAREACCRECCARA (SEQ ID NO:81). In illustrative embodiments, the epitope tag can be the HisX6 tag (SEQ ID NO:77). In some embodiments, the CARs can be cross-linked and activated by adding soluble antibodies or antibody mimetics that bind the epitope tag, or in illustrative embodiments by adding cells, also referred to herein as universal feeder cells, expressing antibodies or antibody mimetics on their surfaces that bind the epitope tag,. In some embodiments, the same universal feeder cells, for example universal feeder cells expressing an anti-HisX6 antibody, can be used with cells that express CARs that bind to different antigens but that include the same epitope tag, for example HisX6. In some embodiments, the CARs can be cross-linked and activated by adding mRNA that encodes for one or more antibodies or antibody mimetics that bind the epitope tag. The mRNA may encode for antibodies or antibody mimetics that are soluble, membrane-bound, or both soluble and membrane-bound in some embodiments.

Provided herein in one aspect is a cell formulation (i.e., delivery composition), comprising a delivery solution formulated with tumor infiltrating lymphocytes (TILs) and/or modified or unmodified lymphocytes, in illustrative embodiments T cells and/or NK cells, wherein the cell formulation is compatible with, effective for, and/or adapted for subcutaneous or intramuscular delivery. In some embodiments for any of the cell formulations provided herein, the cell formulation is localized subcutaneously, or most of the cell formulation is localized subcutaneously, in a subject. In some embodiments, the cell formulation is localized subcutaneously or intramuscularly, or most of the cell formulation is localized subcutaneously or intramuscularly, in a subject. In some embodiments, wherein the cell formulation comprises TILs, the cell formulation can further comprise modified lymphocytes modified by either or both, being associated with a recombinant nucleic acid vectors, in illustrative embodiments a RIP, comprising a polynucleotide comprising one or more transcriptional units operatively linked to a promoter active in T cells and/or NK cells, or by being genetically modified with the polynucleotide, wherein the one or more transcriptional units encode a first polypeptide comprising a first CAR. In some embodiments, wherein the cell formulation comprises TILs, the cell formulation further comprises a source of a tumor antigen recognized by the TILs. In some embodiments, the TILs are contacted with a nucleic acid vector.

In addition to any of the method aspects and embodiments provided herein, further provided herein are use aspects and embodiments, comprising use of a kit for performing the method, or use of nucleic acid vectors, in illustrative embodiments RIPs, in the manufacture of a kit for performing the method, wherein the use of the kit is to perform the steps of the method aspects or embodiments.. Similarly, for any use aspects and embodiments provided herein, further provided herein are method aspects and embodiments, comprising the method as recited in the use aspects or embodiments.

The following non-limiting examples are provided purely by way of illustration of exemplary embodiments, and in no way limit the scope and spirit of the present disclosure. Furthermore, it is to be understood that any inventions disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

### EXAMPLES

### Example 1 Preparation of a Phage Display Library of scFvs From Human CD19+ PBMCs

### Isolation of Human Immunoglobulin Heavy and Kappa Light Antibody Chains

Frozen human PBMCs previously isolated from the whole blood of 13 anonymous donors with informed consent (San Diego Blood Bank) were thawed and pooled (1.5 × 10⁸ PBMCs). CD19⁺ PBMCs were isolated (1.2 × 10⁷) using the EasySep™ Human CD19 Positive Selection Kit II (Stemcell Technologies, #17854). RNA (7 µg) was isolated from the CD19⁺ PBMCs using the RNeasy Plus Mini Prep Kit (Qiagen). First strand cDNA was synthesized from the mRNA transcripts using anchored oligo d(T)20 with SuperScript IV (Invitrogen). Antibody variable chains were then amplified by PCR using a degenerate primer pool specific for the human heavy chain and kappa light chain. The degenerate primer pool included 6 forward and 3 reverse primer sequences directed to the heavy chain, and 6 forward and 5 reverse primer sequences directed to the kappa light chain. 2 primers with distinct adapters to allow for directional cloning were used for each sequence.

### Assembly of the Xphage2 scFv Library

Each of the heavy chain PCR products and each of the kappa chain PCR products were gel purified and cloned into 2 orientation-specific cloning vectors. Heavy and kappa light chains were then seamlessly assembled with linkers into full scFvs within a phagemid backbone to generate the Xphage.2 library. Six unique sequences were used to link the heavy and light antibody chains to generate the library: GGSSRSS (“L1”, SEQ ID NO:673); (G₄S)₂ (“L2”, SEQ ID NO:674); (G₄S)₃ (“L3”, SEQ ID NO:673); (G₄S)₄ (“L4”, SEQ ID NO:372); (G₄S)₅ (“L5”, SEQ ID NO:675); and (G₄S)₆ (“L6”, SEQ ID NO:64).

### Estimation of Library Diversity

The diversity of the individual heavy chain and kappa light chain libraries was determined by a combination of NGS and bioinformatics. Briefly, the heavy and light chain libraries were sequenced using paired-end sequencing (Illumina). Paired-end reads were pre-processed by quality filtering with Trimmomatic v0.36, followed by read clipping and pairing. Clustering was then performed using custom scripts. The distribution of reads was used to reconstruct the diversity of the starting population by Recon (Kaplinsky & Arnaout 2016). The estimated total diversities of the heavy and light chain libraries are 3.7 × 10⁶ and 1.5 × 10⁶, respectively as shown in Table 6. The estimated total diversity of the Xphage2 scFv library was 6.7 ×10¹³ ((Kappa, 1.5×10⁶) × (Heavy, 3.7×10⁶) × (linkers, 6) × (orientations, 2)).

TABLE 6

Estimated diversity of the heavy and light (kappa) chain libraries

| | Heavy | Light (Kappa) |
|---|---|---|
| Initial number of reads | 29,940,489 | 23,173,768 |
| Number of sequences after preprocessing | 20,972,780 (70%) | 19,670,684 (85%) |
| Number of observed clusters | 2,074,152 | 525,995 |
| Estimated number of non-observed clusters | 1,649,947 | 1,001,427 |
| Estimated total diversity | 3,724,099 | 1,527,422 |

### Example 2 Isolation of Recombinant Anti-Idiotype Antibodies From the Xphage2 scFv Library

Monoclonal antibodies were used as bait (Ab1) to pan the Xphage2 scFv library and identify antibodies (Ab2) specific for the idiotype of the bait (Ab1). Two separate panning experiments were performed using different baits, Ab1.1in Panning Experiment 1 (PE1), and Ab1.2 in Panning Experiment 2 (PE2). Both baits were anti-EGFR (cetuximab). One bait was manufactured in CHO Chinese hamster cells (Selleck Chemicals, A2000) while the other was manufactured in SP2/0 mouse myeloma cells (Merck, 226667). Each of the antibodies used as baits was of the IgG1 isotype.

Liquid phase biopanning of the Xphage2 scFv library was performed using Ab1 antibodies coupled to magnetic Dynabeads. In Panning Experiment 1, five rounds of panning were performed using Dynabeads™ Protein A (Thermo Fisher, #10001D) and Dynabeads™ M-270 Epoxy (Thermo Fisher, #14301) in parallel. Briefly, in the first round panning polyclonal human IgG antibodies coupled to the Protein A or M-270 Epoxy Dynabeads that had been blocked with PBS + 2% milk and washed by PBS were 1:1 volume mixed with phage particles and incubated in PBS + 2% milk for 1 hour at room temperature before the supernatant was collected in a new tube. This pre-clear step was performed to remove non-specific phage that bound to the beads and human IgG monoclonal antibody (CrownBio, Cat. C0001). Dynabeads coupled to Ab1.1 and blocked with PBS + 2% milk were then added to the pre-cleared phage and incubated for 1.5 hours at room temperature before being washed 10 times with PBST (PBS containing 0.05% Tween-20) and 2 times with PBS. Phage bound to the Ab1.1 coupled beads were eluted twice in a total of 1.2 ml 0.1 M glycine-HCL, pH2.2 (“Elution Buffer”) and 0.6 ml 1 M Tris-HCL, pH8.0 (“Neutralization Buffer). The eluted phage was then used to infect TG1 bacteria (Lucigen, 60502-2) grown to an OD600 of 0.5 in 40 ml 2×YT medium. After a 1 hour incubation at 37° C., 1 ml of bacteria was removed for analysis (10 µl was used for phage titer determination and 50 ul was used for picking single colony by serial dilutions and the remainder was frozen for stock). The remaining TG1 were pelleted, resuspended in 1.5 ml 2xYT+Amp and spread on a 2YT+AMP+1%w/v glucose plate and incubated overnight at 37° C. The bacteria were then collected and used to inoculate 80 ml 2YT+AMP+1%w/v glucose and incubated at 37° C. until the OD600 was 0.5. Hyperphage M13 K07ΔpIII (Progen, PRHYPE) were added at an MOI of 20 and the mixture was incubated for 1 hour at 37° C. The bacteria were then collected, resuspended in 80 ml of 2YT+Amp+Kan medium with 0.1 mM IPTG, and cultured overnight at 30° C., 230 rpm. The supernatant was then collected, and phage particles were precipitated in a PEG8000/NaCl solution and resuspended in 1.8 ml PBS (the “phage output”) and tittered.

Subsequent rounds of panning were performed similarly using the phage output of the prior panning round, but with some modifications. In the fourth and fifth rounds of panning, a second pre-clear step was performed using anti-humanCD20 IgG antibodies (rituximab) (Selleck Chemicals, A2009) coupled to Dynabeads after the phage supernatant was pre-cleared with the human IgG polyclonal antibody (Thermofisher, Cat.02-7102). In the fourth panning round, PBS + 2% HSA was used in place of PBS + 2% milk to block the beads. For panning round 5b, 1 ml PBS containing 20 ug/ml EGFR-His was used to specifically compete and elute phage that displayed an Ab2 that recognizes the antigen (EGFR) binding site of Ab1.1.

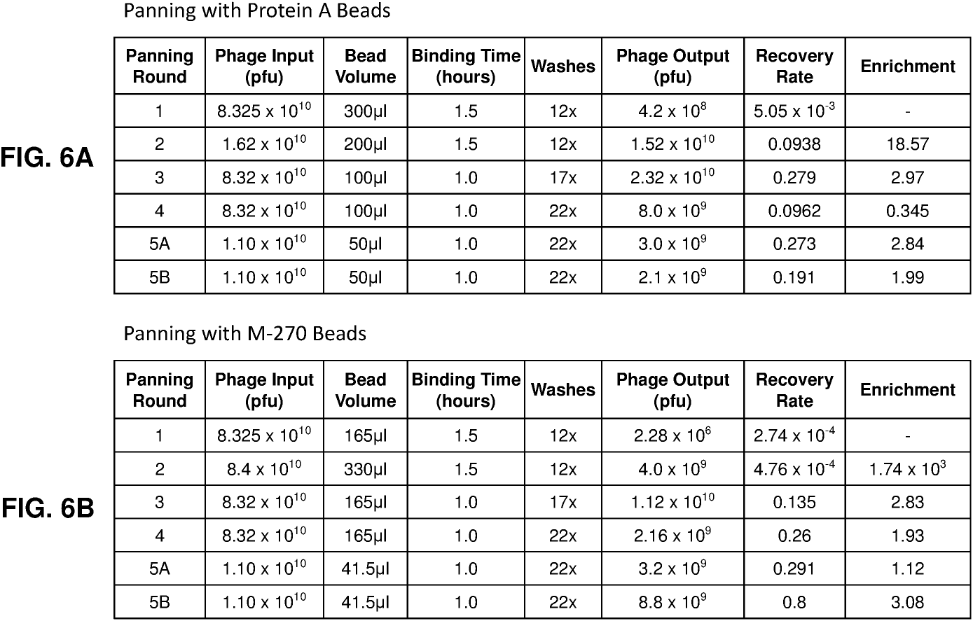

FIG. 6A and FIG. 6B show, for each round of panning in PE1, the phage input (pfu), bead volume, number of washes, phage output (pfu), recovery rate and enrichment from the prior round for panning with Dynabeads™ Protein A and Dynabeads™ M-270 Epoxy, respectively. The pool ELISA result showed that panning with Dynabeads M-270 Epoxy showed better enrichment than Dynabeads Protein A after 3 rounds panning.

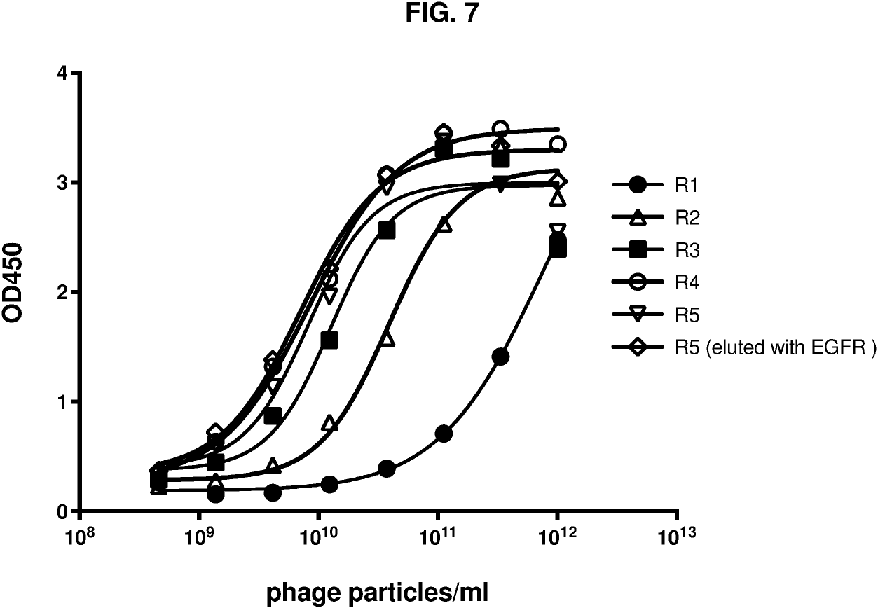

The phage output from each panning round was analyzed for Ab1.1 binding by ELISA. The wells of 96-well plates were coated with Ab1.1, and a serial dilution of the phage output from each panning round was added. Bound phage were detected using HRP conjugated anti-M13 antibody (Hangzhous Hua’an Biotech, #EM1902-18) and 1-Step™ Ultra TMB-ELISA Substrate Solution (Thermo Fisher Scientific, #34028). The absorbance at 450 nm was measured and data was processed using GraphPad Prism 8. The results of panning with Ab1.1 coupled to Dynabeads™ M-270 Epoxy is shown in FIG. 7. As shown in the graph, phage displaying antibodies that bind Ab1.1 were successfully isolated in each panning round. Furthermore, there was a large enrichment of Ab1.1-specific phage between rounds 1 and 2, and between rounds 2 and 3. Little enrichment of Ab1.1-specific phage was seen in subsequent rounds. Phage displaying antibodies that bind Ab1.1 were also isolated in each round of panning with Ab1.1 coupled to Dynabeads ™ Protein A.

Panning Experiment 2 (PE2) was performed using substantially similar methods to those used in PE1. Ab1.2 coupled to Dynabeads ™ M-270 Epoxy was used as bait for a total of 3 rounds of panning. PBS + 2% milk was used for all blocking steps. Prior to the first round of panning, the phage library was pre-cleared sequentially with human IgG polyclonal antibodies, anti-humanCD20 IgG antibodies (rituximab) (Selleck Chemicals, A2009), and anti-human Biotin-CD3 (OKT3) (Biolegend, #317320. In subsequent rounds, pre-clearing was performed using human IgG polyclonal antibodies without any subsequent pre-clearing steps. 0.1 M glycine-HCL, pH2.2 was used to elute Ab1.2-bound phage in each round. Similar to the results for PE1, phage displaying antibodies that bind Ab1.2 were successfully isolated in each panning round.

### Example 3. Identification of Individual Recombinant Anti-Idiotype Antibodies Specific For the Antibody Variable Domain to Which the Anti-Idiotype Was Raised

Individual anti-idiotype scFvs were cloned from phage pools and tested by ELISA for specific biding to the idiotype of the antibody that was used as bait for panning. Sequences of the scFvs were determined by Sanger sequencing.

281 clones from Panning Experiment 1 (PE1) (89 clones from the 4^{th} panning round, and 192 clones from the 5^{th} panning round) and 95 clones from Panning Experiment 2 (PE2) (30 clones from the 2^{nd} panning round, and 65 clones from the 3^{rd} panning round) were expanded in TG1 and packaged with Hyperphage in 96 deep well plate. Monoclonal phage ELISAs were performed in which microtiter plates were coated with Ab1.1 (for PE1) or Ab1.2 (for PE2) or Control antibody, and individual phage clones were added to each well. Binding was detected with anti-M13-HRP and absorbance measured at OD450nm.

The results of the monoclonal phage ELISAs are shown in Tables 2, 3, 4, and 5. The columns indicate whether binding was to the bait (Ab1.1 or Ab1.2) or the Control antibody (rituximab). Binding is shown as the OD450nm. “Ratio” is the ratio of binding of bait to Control antibody. Because the baits and Control antibody are each chimeric monoclonal antibodies of the human IgG1 isotype, effective binding of phage to a bait but not the Control (determined as a Ratio > 3.0) indicate that the phage expresses an anti-idiotype antibody specific for the idiotype of the bait (Ab1.1 or Ab1.2). 24 clones from the 4^{th} panning round and 40 clones from the 5^{th} panning round of PE1 were identified as Ab1.1 anti-idiotype antibodies. Similarly, 23 clones from the 2^{nd} panning round and 54 clones from the 3^{rd} panning round of PE2 were identified as Ab1.2 anti-idiotype antibodies.

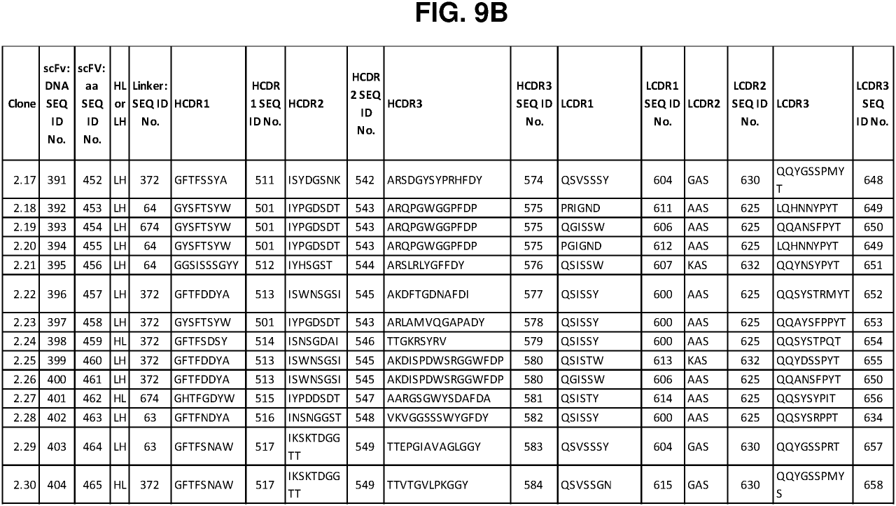

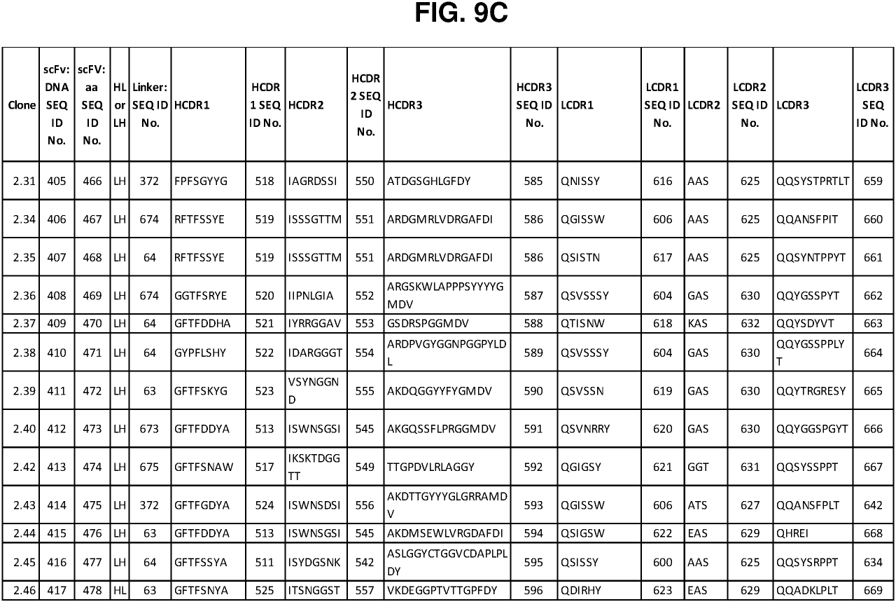

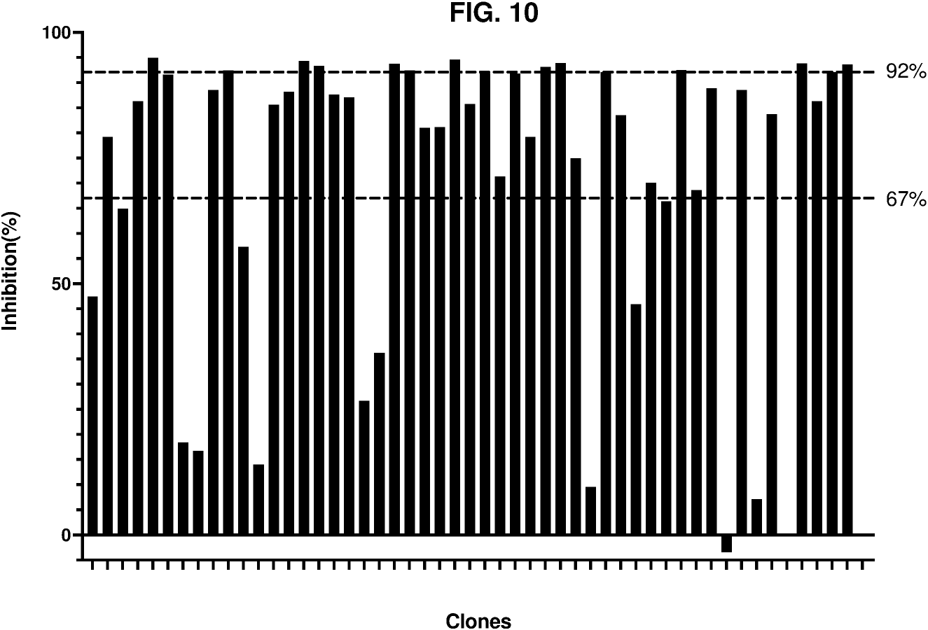

Sequence data for each unique scFv identified in PE1 is shown in FIG. 8 and the sequence data for each unique scFv identified in PE2 is shown in FIGS. 9A, 9B, and 9C. Individual clones are shown in each row. Shown in columns are the SEQ ID NOs. of the DNA and amino acid of the full scFvs, the orientation of the heavy and light chains, and the SEQ ID NO. of the linkers. Panning of the Xphage2 scFv library using Ab1.1 as bait in PE1 yielded 19 unique scFv clones. Similarly, panning of the Xphage2 scFv library using Ab1.2 as bait in PE2 yielded 42 unique scFv clones. The amino acid sequences of each CDR and their SEQ ID NO. are also shown. As shown in the tables, some clones shared a HCDR1, HCDR2, HCDR3, LCDR1, LCDR2, and/or LCDR3. Furthermore, some clones shared the same sequences for all of their CDRs. CDRs were determined using VBASE2 at www.vbase2.org.

The isolation of many unique scFv clones in PE1 and PE2 demonstrate that Xphage2 library and panning methods described herein can be used effectively to identify scFvs as well as antibody heavy and light chains that are capable of binding to the idiotypes of other antibodies.

### Example 4. Identification of Individual Recombinant Anti-Idiotype Antibodies That Recognize Specific Regions of the Idiotype of the Antibodies to Which the Anti-Idiotype Antibodies Were Raised

The anti-idiotype antibodies identified in Example 3 were further screened by competitive ELISA to identify both anti-idiotype antibodies specific to the antigen binding site (ABS) of their cognate Ab1 antibodies, and other anti-idiotype antibodies specific to epitopes of the idiotype that are outside of the ABS.

Cetuximab anti-EGFR antibody was used as bait (Ab1.2) in Panning Experiment 2. To identify anti-idiotype antibodies that bind the ABS of cetuximab and other antibodies that bind outside of the ABS, soluble EGFR (Acrobiosystems, Cat# EGR-H5222) was added to compete with monoclonal phage for binding to cetuximab in ELISAs. Briefly, microtiter plates were coated with Ab1.2 and 50 individual phage clones displaying anti-idiotype antibodies identified in PE2 were added to wells in duplicate. For each sample plated in duplicate, soluble EGFR at a final concentration of 1 µg/ml was added to one of the wells. Binding of monoclonal phage was detected with anti-M13-HRP and absorbance measured at OD450nm.

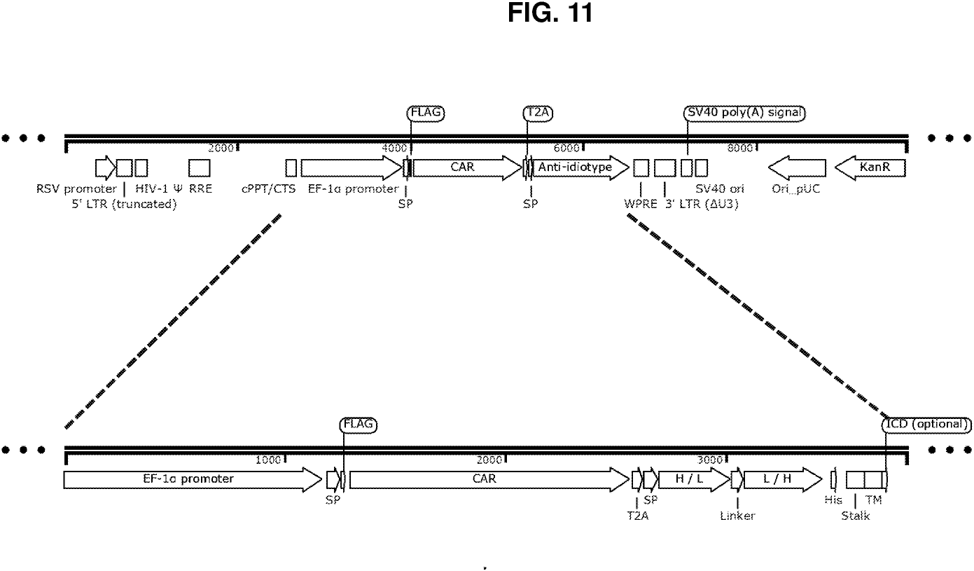

Results of the competitive ELISA for 50 monoclonal phage are shown in FIG. 10. Inhibition of binding (Inhibition%) was calculated as (OD in the absence of EGFR — OD in the presence of EGFR) / (OD in the absence of EGFR — OD of plate background) × 100. Soluble EGFR will compete with antibodies that recognize the ABS for binding to cetuximab. Therefore, Inhibition% will be high if the anti-idiotype antibody recognizes the ABS and low if the anti-idiotype antibody recognizes a region of cetuximab outside of the ABS. Monoclonal phage with an Inhibition% greater than the 75^{th} percentile of Inhibition% (92%) are selected to be anti-idiotype antibodies that recognize the ABS. Of the 50 phage shown in this screen, 13 clones were determined to be anti-idiotype antibodies that recognize the ABS of cetuximab. In contrast, monoclonal phage with an Inhibition% less than the 25^{th} percentile of Inhibition% (67%) are believed to be anti-idiotype antibodies that recognize regions of the variable domain that are outside of the ABS. Of the 50 phage shown in this screen, 6 clones were determined to be anti-idiotype antibodies that recognize regions of the variable domain of cetuximab that are outside of the ABS. The results in this example demonstrate that both anti-idiotype antibodies that recognize the ABS of a target antibody, and anti-idiotype antibodies that recognize an epitope of the idiotype of a target antibody that is outside of the ABS, can be identified using the methods described herein. Similar methods can also be used to identify antibody mimetics that bind the ABS of a target antibody or antibody mimetic.

### Example 5 Surface Expression of Anti-Idiotype Polypeptide on Mammalian Cells

Select anti-idiotype antibodies identified in Example 3 were cloned into vectors such that the transcriptional unit encoded a first polypeptide comprising a CAR and a second polypeptide comprising the anti-idiotype polypeptide. These vectors were transiently transfected and expressed on mammalian cells.

As shown in FIG. 11, the vector into which the scFvs were cloned encoded a CD19 CAR comprised of an anti-CD19scFv, a CD8 stalk and transmembrane region, and an intracellular domain from CD3z followed by T2A and an anti-idiotype scFv fused to a “STMICD” comprised of a Histidine tag (optional), a stalk, a transmembrane domain (TM), and an intracellular domain (optional) (“ICD”). A number of different STMICDs were tested. STMICD1 comprised the hinge, TM and 8 amino acids of the ICD PDGFRβ (SEQ ID NO: 676). STMICD2 comprised the hinge, TM and 9 amino acids of the ICD CD28 (SEQ ID NO: 677). STMICD3 comprised the hinge, TM and full ICD of CD28 (SEQ ID NO: 678). STMICD4 comprised the hinge, TM, and full ICD of CD28 fused to the full ICD of CD80 (SEQ ID NO: 679).

The expression vectors were transiently transfected into mammalian cells using Lipofectamine 2000 (Thermo Fisher Scientific), cultured at 37° C. and 5% CO₂ for 48 hours, harvested, and stained for FACs analysis with anti-His (BioLegend, 362607) and cetuximab (Selleck Chemicals, A2000), which was the Ab1 used as bait to identify the anti-idiotype scFvs.

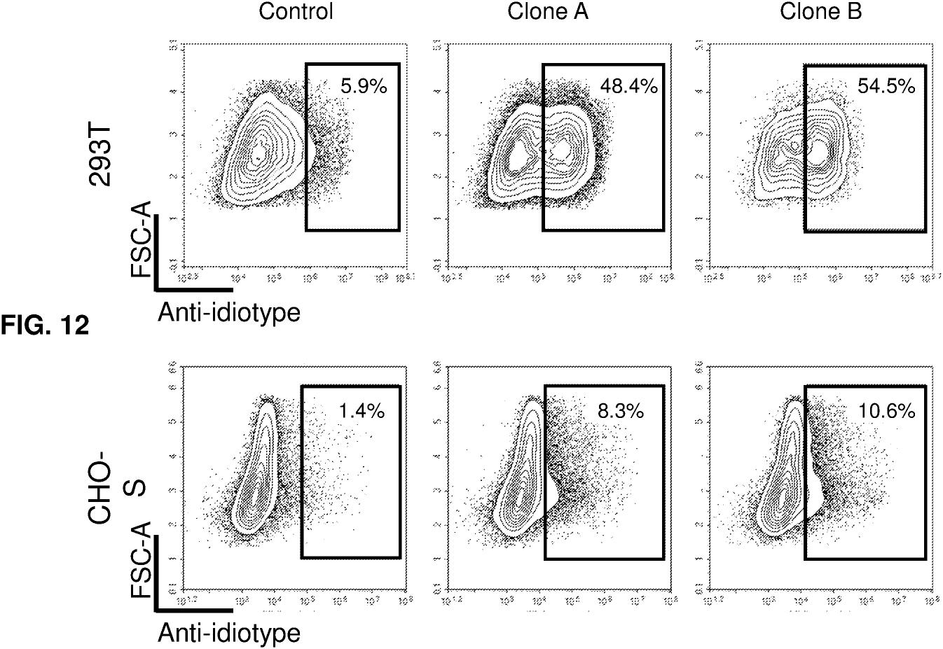

Expression of anti-idiotype polypeptides on the surface of mammalian cells as detected by cetuximab is shown in FIG. 12. Vectors for Clone A and Clone B encoded unique scFvs, while the Control vector lacked an insert encoding an anti-idiotype polypeptide. Both Clone A and Clone B comprised STMICD4 in this experiment. As shown in FIG. 12, the anti-idiotype polypeptide was expressed as detected by Ab1 on 48.4% (Clone A) and 54.5% (Clone B) of transiently transfected 293T cells. Similarly, anti-idiotype polypeptide was expressed and detected on 8.3% (Clone A) and 10.6% (Clone B) of transiently transfected CHO-S cells. In a separate experiment, surface expression of the anti-idiotype polypeptide was also detected on cells when STMICD4 was replaced with each of STMICD1, STMICD2, and STMICD3 demonstrating that a variety of stalks, TMS, and ICDs could be used to achieve surface expression.

This example demonstrates that the methods and compositions disclosed herein can be used to generate anti-idiotype polypeptides that can be expressed on the surface of mammalian cells and are capable of binding to the variable domains of their target antibodies.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

TABLE 1

Parts, names, and amino acid sequences for domains of lymphoproliferative parts P1-P2, P1, P2, P3, and P4

| | | |
|---|---|---|
| Part | Name | Amino Acid Sequence |
| M001 | eTAG IL7RA Ins PPCL (interleukin 7 receptor) | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPEINNSSGEMDPILLPPCLTISILSFFSVALLVILACVL (SEQ ID NO:84) |
| M002 | eTAG IL7RA Ins PPCL (interleukin 7 receptor) | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQPEINNSSGEMDPILLPPCLTISILSFFSVALLVILACVL (SEQ ID NO:85) |
| M007 | Myc Tag LMP1 NC_007605_1 | MEQKLISEEDLEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWM (SEQ ID NO:86) |
| M008 | Myc LMP1 NC_007605_1 | MEQKLISEEDLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWM (SEQ ID NO:87) |
| M009 | LMP1 NC_007605_1 | MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWM (SEQ ID NO:88) |
| M010 | LMP1 NC_007605_1 | MSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWM (SEQ ID NO:89) |
| M012 | eTAG CRLF2 transcript variant 1 NM_022148_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGAETPTPPKPKLSKCILISSLAILLMVSLLLLSLW (SEQ ID NO:90) |
| M013 | eTAG CRLF2 transcript variant 1 NM_022148_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQAETPTPPKPKLSKCILISSLAILLMVSLLLLSLW (SEQ ID NO:91) |
| M018 | eTAG CSF2RB NM_000395_2 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGTESVLPMWVLALIEIFLTIAVLLAL (SEQ ID NO:92) |
| M019 | eTAG CSF2RB NM_000395_2 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTESVLPMWVLALIEIFLTIAVLLAL (SEQ ID NO:93) |
| M024 | eTAG CSF3R transcript variant 1 NM_000760_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGTPEGSELHIILGLFGLLLLLNCLCGTAWLCC (SEQ ID NO:94) |
| M025 | eTAG CSF3R transcript variant 1 NM_000760_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTPEGSELHIILGLFGLLLLLNCLCGTAWLCC (SEQ ID NO:95) |
| M030 | eTAG EPOR transcript variant 1 NM_000121_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGTPSDLDPCCLTLSLILVVILVLLTVLALLS (SEQ ID NO:96) |
| M031 | eTAG EPOR transcript variant 1 NM_000121_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTPSDLDPCCLTLSLILVVILVLLTVLALLS (SEQ ID NO:97) |
| M036 | eTAG GHR transcript variant 1 NM_000163_4 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGTLPQMSQFTCCEDFYFPWLLCIIFGIFGLTVMLFVFLFS (SEQ ID NO:98) |
| M037 | eTAG GHR transcript variant 1 NM_000163_4 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTLPQMSQFTCCEDFYFPWLLCIIFGIFGLTVMLFVFLFS (SEQ ID NO:99) |
| M042 | eTAG truncated after Fn F523C IL27RA NM_004843_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGHLPDNTLRWKVLPGILCLWGLFLLGCGLSLA (SEQ ID NO:100) |
| M043 | eTAG truncated after Fn F523C IL27RA NM_004843_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQHLPDNTLRWKVLPGILCLWGLFLLGCGLSLA (SEQ ID NO:101) |
| M048 | eTAG truncated after Fn S505N MPL NM_005373_2 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGETATETAWISLVTALHLVLGLNAVLGLLLL (SEQ ID NO:102) |
| M049 | eTAG truncated after Fn S505N MPL NM_005373_2 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQETATETAWISLVTALHLVLGLNAVLGLLLL (SEQ ID NO:103) |
| E006 | eTag 0A JUN NM_002228_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWK |
| | | YADAGHVCHLCHPNCTYGCTGPGLEGCPTNGLERIARLEEKVKTLKAQNSELASTANMLREQVAQLKQKV (SEQ ID NO:104) |
| E007 | eTag 1A JUN NM_002228_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKVA (SEQ ID NO:105) |
| E008 | eTag 2A JUN NM_002228_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKVAA (SEQ ID NO:106) |
| E009 | eTag 3A JUN NM_002228_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKVAAA (SEQ ID NO:107) |
| E010 | eTag 4A JUN NM_002228_3 | MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGLERIARLEEKVKTLKAQNSELASTANMLREQVAQLKQKVAAAA (SEQ ID NO:108) |
| E011 | Myc Tag 0A JUN NM_002228_3 | MTILGTTFGMVFSLLQVVSGEQKLISEEDLLERIARLEEKVKTLKAQNSELASTANMLREQVAQLKQKV (SEQ ID NO:109) |
| E012 | Myc Tag 1A JUN NM_002228_3 | MTILGTTFGMVFSLLQVVSGEQKLISEEDLLERIARLEEKVKTLKAQNSELASTANMLREQVAQLKQKVA (SEQ ID NO:110) |
| E013 | Myc Tag 2A JUN NM_002228_3 | MTILGTTFGMVFSLLQVVSGEQKLISEEDLLERIARLEEKVKTLKAQNSELASTANMLREQVAQLKQKVAA (SEQ ID NO:111) |
| E014 | Myc Tag 3A JUN NM_002228_3 | MTILGTTFGMVFSLLQVVSGEQKLISEEDLLERIARLEEKVKTLKAQNSELASTANMLREQVAQLKQKVAAA (SEQ ID NO:112) |
| E015 | Myc Tag 4A JUN NM_002228_3 | MTILGTTFGMVFSLLQVVSGEQKLISEEDLLERIARLEEKVKTLKAQNSELASTANMLREQVAQLKQKVAAAA (SEQ ID NO:113) |
| E016 | 0A JUN NM_002228_3 | LERIARLEEKVKTLKAQNSELASTANMLREQVAQLKQKV |
| T001 | CD2 transcript variant 1 NM_001328609_1 | LIIGICGGGSLLMVFVALLVFYI (SEQ ID NO:114) |
| T002 | CD3D transcript variant 1 NM_000732_4 | GIIVTDVIATLLLALGVFCFA (SEQ ID NO:115) |
| T003 | CD3E NM_000733_3 | VMSVATIVIVDICITGGLLLLVYYWS (SEQ ID NO:116) |
| T004 | CD3G NM_000073_2 | GFLFAEIVSIFVLAVGVYFIA (SEQ ID NO:117) |
| T005 | CD3Z CD247 transcript variant 1 NM_198053_2 | LCYLLDGILFIYGVILTALFL (SEQ ID NO:118) |
| T006 | CD4 transcript variant 1 and 2 NM_000616_4 | MALIVLGGVAGLLLFIGFIGLGIFF (SEQ ID NO:119) |
| T007 | CD8A transcript variant 1 NM_001768_6 | IYIWAPLAGTCGVLLLSLVIT (SEQ ID NO:120) |
| T008 | CD8B transcript variant 2 NM_17223 | LGLLVAGVLVLLVSLGVAIHLCC (SEQ ID NO:121) |
| T009 | CD27 NM_001242_4 | ILVIFSGMFLVFTLAGALFLH (SEQ ID NO:122) |
| T010 | CD28 transcript variant 1 NM_006139_3 | FWVLVVVGGVLACYSLLVTVAFIIFWV (SEQ ID NO:123) |
| T011 | CD40 transcript variant 1 and 6 NM_001250_5 | ALVVIPIIFGILFAILLVLVFI (SEQ ID NO:124) |
| T012 | CD79A transcript variant 1 NM_001783_3 | \|\|TAEGIILLFCAVVPGTLLLF (SEQ ID NO:125) |
| T013 | CD79B transcript variant 3 NM_001039933_2 | GIIMIQTLLIILFIIVPIFLLL (SEQ ID NO:126) |
| T014 | CRLF2 transcript variant 1 NM_0221483 | FILISSLAILLMVSLLLLSLW (SEQ ID NO:127) |
| T015 | CRLF2 transcript variant 1 NM_022148_3 | CILISSLAILLMVSLLLLSLW (SEQ ID NO:128) |
| T016 | CSF2RA transcript variant 7 and 8 NM_001161529_1 | NLGSVYIYVLLIVGTLVCGIVLGFLF (SEQ ID NO:129) |
| T017 | CSF2RB NM_000395_2 | MWVLALIVIFLTIAVLLAL (SEQ ID NO:130) |
| T018 | CSF2RB NM_000395_2 | MWVLALIEIFLTIAVLLAL (SEQ ID NO:131) |
| T019 | CSF3R transcript variant 1 NM_000760_3 | IILGLFGLLLLLTCLCGTAWLCC (SEQ ID NO:132) |
| T020 | CSF3R transcript variant 1 NM_000760_3 | IILGLFGLLLLLNCLCGTAWLCC (SEQ ID NO:133) |
| T021 | EPOR transcript variant 1 NM_000121_3 | LILTLSLILVVILVLLTVLALLS (SEQ ID NO:134) |
| T022 | EPOR transcript variant 1 NM_000121_3 | CCLTLSLILVVILVLLTVLALLS (SEQ ID NO:135) |
| T023 | FCER1G NM_004106_1 | LCYILDAILFLYGIVLTLLYC (SEQ ID NO:136) |
| T024 | FCGR2C NM_201563_5 | IIVAVVTGIAVAAIVAAVVALIY (SEQ ID NO:137) |
| T025 | FCGRA2 transcript variant 1 NM_001136219_1 | IIVAVVIATAVAAIVAAVVALIY (SEQ ID NO:138) |
| T026 | GHR transcript variant 1 NM_000163_4 | FPWLLIIIFGIFGLTVMLFVFLFS (SEQ ID NO:139) |
| T027 | GHR transcript variant 1 NM_000163_4 | FPWLLCIIFGIFGLTVMLFVFLFS (SEQ ID NO:140) |
| T028 | ICOS NM_012092.3 | FWLPIGCAAFVVVCILGCILI (SEQ ID NO:141) |
| T029 | IFNAR1 NM_000629_2 | IWLIVGICIALFALPFVIYAA (SEQ ID NO:142) |
| T030 | IFNAR2 transcript variant 1 NM_207585_2 | IGGIITVFLIALVLTSTIVTL (SEQ ID NO:143) |
| T031 | IFNGR1 NM_000416_2 | SLWIPVVAALLLFLVLSLVFI (SEQ ID NO:144) |
| T032 | IFNGR2 transcript variant 1 NM_001329128_1 | VILISVGTFSLLSVLAGACFF (SEQ ID NO:145) |
| T033 | IFNLR1 NM_170743_3 | FLVLPSLLILLLVIAAGGVIW (SEQ ID NO:146) |
| T034 | IL1R1 transcript variant 2 NM_001288706_1 | HMIGICVTLTVIIVCSVFIYKIF (SEQ ID NO:147) |
| T035 | IL1RAP transcript variant 1 NM_002182_3 | VLLVVILIVVYHVYWLEMVLF (SEQ ID NO:148) |
| T036 | IL1RL1 transcript variant 1 NM_016232.4 | IYCIIAVCSVFLMLINVLVII (SEQ ID NO:149) |
| T037 | IL1RL2 NM_003854.2 | AYLIGGLIALVAVAVSVVYIY (SEQ ID NO:150) |
| T038 | IL2RA transcript variant 1 NM_000417_2 | VAVAGCVFLLISVLLLSGL (SEQ ID NO:151) |
| T039 | IL2RB transcript variant 1 NM_000878_4 | IPWLGHLLVGLSGAFGFIILVYLLI (SEQ ID NO:152) |
| T040 | IL2RG NM_000206_2 | VVISVGSMGLIISLLCVYFWL (SEQ ID NO:153) |
| T041 | IL3RA transcript variant 1 and 2 NM_002183_3 | TSLLIALGTLLALVCVFVIC (SEQ ID NO:154) |
| T042 | IL4R transcript variant 1 NM_000418_3 | LLLGVSVSCIVILAVCLLCYVSIT (SEQ ID NO:155) |
| T043 | IL5RA transcript variant 1 NM_000564_4 | FVIVIMATICFILLILSLIC (SEQ ID NO:156) |
| T044 | IL6R transcript variant 1 NM_000565_3 | TFLVAGGSLAFGTLLCIAIVL (SEQ ID NO:157) |
| T045 | IL6ST transcript variant 1 and 3 NM_002184_3 | AIVVPVCLAFLLTTLLGVLFCF (SEQ ID NO:158) |
| T046 | IL7RA NM_002185_3 | ILLTISILSFFSVALLVILACVL (SEQ ID NO:159) |
| T047 | IL7RA Ins PPCL (interleukin 7 receptor) | ILLPPCLTISILSFFSVALLVILACVL (SEQ ID NO:160) |
| T048 | IL9R transcript variant 1 NM_002186_2 | GNTLVAVSIFLLLTGPTYLLF (SEQ ID NO:161) |
| T049 | IL10RA transcript variant 1 NM_001558_3 | VIIFFAFVLLLSGALAYCLAL (SEQ ID NO:162) |
| T050 | IL10RB NM_000628_4 | WMVAVILMASVFMVCLALLGCF (SEQ ID NO:163) |
| T051 | IL11RA NM_001142784_2 | SLGILSFLGLVAGALALGLWL (SEQ ID NO:164) |
| T052 | IL12RB1 transcript variant 1 and 4 NM_005535_2 | WLIFFASLGSFLSILLVGVLGYLGL (SEQ ID NO:165) |
| T053 | IL12RB2 transcript variant 1 and 3 NM_001559_2 | WMAFVAPSICIAIIMVGIFST (SEQ ID NO:166) |
| T054 | IL13RA1 NM_001560_2 | LYITMLLIVPVIVAGAIIVLLLYL (SEQ ID NO:167) |
| T055 | IL13RA2 NM_000640_2 | FWLPFGFILILVIFVTGLLL (SEQ ID NO:168) |
| T056 | IL15RA transcript variant 4 NM_001256765_1 | VAISTSTVLLCGLSAVSLLACYL (SEQ ID NO:169) |
| T057 | IL17RA NM_014339_6 | VYWFITGISILLVGSVILLIV (SEQ ID NO:170) |
| T058 | IL17RB NM_018725_3 | LLLLSLLVATWVLVAGIYLMW (SEQ ID NO:171) |
| T059 | IL17RC transcript variant 1 NM_153460_3 | WALVWLACLLFAAALSLILLL (SEQ ID NO:172) |
| T060 | IL17RD transcript variant 2 NM_017563_4 | AVAITVPLVVISAFATLFTVM (SEQ ID NO:173) |
| T061 | IL17RE transcript variant 1 NM_153480_1 | LGLLILALLALLTLLGVVLAL (SEQ ID NO:174) |
| T062 | IL18R1 transcript variant 1 NM_003855_3 | GMIIAVLILVAVVCLVTVCVI (SEQ ID NO:175) |
| T063 | IL18RAP NM_003853_3 | GVVLLYILLGTIGTLVAVLAA (SEQ ID NO:176) |
| T064 | IL20RA transcript variant 1 NM_014432_3 | IIFWYVLPISITVFLFSVMGY (SEQ ID NO:177) |
| T065 | IL20RB NM_144717_3 | VLALFAFVGFMLILVVVPLFV (SEQ ID NO:178) |
| T066 | IL21R transcript variant 2 NM_181078_2 | GWNPHLLLLLLLVIVFIPAFW (SEQ ID NO:179) |
| T067 | IL22RA1 NM_021258_3 | YSFSGAFLFSMGFLVAVLCYL (SEQ ID NO:180) |
| T068 | IL23R NM_144701_2 | LLLGMIVFAVMLSILSLIGIF (SEQ ID NO:181) |
| T069 | IL27RA NM_004843_3 | VLPGILFLWGLFLLGCGLSLA (SEQ ID NO:182) |
| T070 | IL27RA NM_004843_3 | VLPGILCLWGLFLLGCGLSLA (SEQ ID NO:183) |
| T071 | IL31RA transcript variant 1 NM_139017_5 | IILITSLIGGGLLILIILTVAYGL (SEQ ID NO:184) |
| T072 | LEPR transcript variant 1 NM_002303_5 | AGLYVIVPVIISSSILLLGTLLI (SEQ ID NO:185) |
| T073 | LIFR NM_001127671_1 | VGLIIAILIPVAVAVIVGVVTSILC (SEQ ID NO:186) |
| T074 | MPL NM_005373_2 | ISLVTALHLVLGLSAVLGLLLL (SEQ ID NO:187) |
| T075 | MPL NM_005373_2 | ISLVTALHLVLGLNAVLGLLLL (SEQ ID NO:188) |
| T076 | OSMR transcript variant 4 NM_001323505_1 | LIHILLPMVFCVLLIMVMCYL (SEQ ID NO:189) |
| T077 | PRLR transcript variant 1 NM_000949_6 | TTVWISVAVLSAVICLIIVWAVAL (SEQ ID NO:190) |
| T078 | TNFRSF4 NM_003327_3 | VAAILGLGLVLGLLGPLAILL (SEQ ID NO:191) |
| T079 | TNFRSF8 transcript variant 1 NM_001243_4 | PVLDAGPVLFWVILVLVVVVGSSAFLLC (SEQ ID NO:192) |
| T080 | TNFRSF9 NM_001561_5 | IISFFLALTSTALLFLLFFLTLRFSVV (SEQ ID NO:193) |
| T081 | TNFRSF14 transcript variant 1 NM_003820_3 | WWFLSGSLVIVIVCSTVGLII (SEQ ID NO:194) |
| T082 | TNFRSF18 transcript variant 1 NM_004195_2 | LGWLTVVLLAVAACVLLLTSA (SEQ ID NO:195) |
| S036 | CD2 transcript variant 1 NM_001328609_1 | TKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN (SEQ ID NO:196) |
| S037 | CD3D transcript variant 1 NM_000732_4 | GHETGRLSGAADTQALLRNDQVYQPLRDRDDAQYSHLGGNWARNK (SEQ ID NO:197) |
| S038 | CD3E NM_000733_3 | KNRKAKAKPVTRGAGAGGRQRGQNKERPPPVPNPDYEPIRKGQRDLYSGLNQRRI (SEQ ID NO:198) |
| S039 | CD3G NM_000073_2 | GQDGVRQSRASDKQTLLPNDQLYQPLKDREDDQYSHLQGNQLRRN (SEQ ID NO:199) |
| S042 | CD4 transcript variant 1 and 2 NM_000616_4 | CVRCRHRRRQAERMSQIKRLLSEKKTCQCPHRFQKTCSPI (SEQ ID NO:200) |
| S043 | CD8A transcript variant 1 NM_001768_6 | LYCNHRNRRRVCKCPRPVVKSGDKPSLSARYV (SEQ ID NO:201) |
| S044 | CD8B transcript variant 2 NM_172213_3 | RRRRARLRFMKQPQGEGISGTFVPQCLHGYYSNTTTSQKLLNPWILKT (SEQ ID NO:202) |
| S045 | CD8B transcript variant 3 NM_172101_3 | RRRRARLRFMKQLRLHPLEKCSRMDY (SEQ ID NO:203) |
| S046 | CD8B transcript variant 5 NM_004931_4 | RRRRARLRFMKQFYK (SEQ ID NO:204) |
| S047 | CD27 NM_001242_4 | QRRKYRSNKGESPVEPAEPCRYSCPREEEGSTIPIQEDYRKPEPACSP (SEQ ID NO:205) |
| S048 | mutated Delta Lck CD28 transcript variant 1 NM_006139_3 | RSKRSRLLHSDYMNMTPRRPGPTRKHYQAYAAARDFAAYRS (SEQ ID NO:206) |
| S049 | CD28 transcript variant 1 NM_006139_3 | RSKRSRLLHSDYMNMTPRRPGPTRKHYQPYAPPRDFAAYRS (SEQ ID NO:207) |
| S050 | CD40 transcript variant 1 and 6 NM_001250_5 | KKVAKKPTNKAPHPKQEPQEINFPDDLPGSNTAAPVQETLHGCQPVTQEDGKESRISVQERQ (SEQ ID NO:208) |
| S051 | CD40 transcript variant 5 NM_001322421_1 | SESSEKVAKKPTNKAPHPKQEPQEINFPDDLPGSNTAAPVQETLHGCQPVTQEDGKESRISVQERQ (SEQ ID NO:209) |
| S052 | CD79A transcript variant 1 NM_001783_3 | RKRWQNEKLGLDAGDEYEDENLYEGLNLDDCSMYEDISRGLQGTYQDVGSLNIGDVQLEKP (SEQ ID NO:210) |
| S053 | CD79B transcript variant 3 NM_001039933_2 | LDKDDSKAGMEEDHTYEGLDIDQTATYEDIVTLRTGEVKWSVGEHPGQE (SEQ ID NO:211) |
| S054 | CRLF2 transcript variant 1 NM_022148_3 | KLWRVKKFLIPSVPDPKSIFPGLFEIHQGNFQEWITDTQNVAHLHKMAGAEQESGPEEPLVVQLAKTEAESPR MLDPQTEEKEASGGSLQLPHQPLQGGDVVTIGGFTFVMNDRSYVAL (SEQ ID NO:212) |
| S057 | CSF2RB NM_000395_2 | RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGD YCFLPGLGPGPLSLRSKPSSPGPGPEIKNLDQAFQVKKPPGQAVPQVPVIQLFKALKQQDYLSLPPWEVNKPGEVC (SEQ ID NO:213) |
| S058 | CSF2RA transcript variant 7 and 8 NM_001161529_1 | KRFLRIQRLFPPVPQIKDKLNDNHEVEDEIIWEEFTPEEGKGYREEVLTVKEIT (SEQ ID NO:214) |
| S059 | CSF2RA transcript variant 9 NM_001161531_1 | KRFLRIQRLFPPVPQIKDKLNDNHEVEDEMGPQRHHRCGWNLYPTPGPSPGSGSSPRLGSESSL (SEQ ID NO:215) |
| S062 | CSF3R transcript variant 1 NM_000760_3 | SPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF (SEQ ID NO:216) |
| S063 | CSF3R transcript variant 3 NM_156039_3 | SPNRKNPLWPSVPDPAHSSLGSWVPTIMEELPGPRQGQWLGQTSEMSRALTPHPCVQDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF (SEQ ID NO:217) |
| S064 | CSF3R transcript variant 4 NM_172313_2 | SPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQAGPPRRSAYFKDQIMLHPAPPNGLLCLFPITSVL (SEQ ID NO:218) |
| S069 | EPOR transcript variant 1 NM_000121_3 | HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS (SEQ ID NO:219) |
| S072 | EPOR transcript variant 1 NM_000121_3 | HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKFLFLVVSDSGISTDYSSGDSQGAQGGLSDGPYSNPYENSLIPAAEPLPPSYVACS (SEQ ID NO:220) |
| S074 | FCER1G NM_004106_1 | RLKIQVRKAAITSYEKSDGVYTGLSTRNQETYETLKHEKPPQ (SEQ ID NO:221) |
| S075 | FCGR2C NM_201563_5 | CRKKRISANSTDPVKAAQFEPPGRQMIAIRKRQPEETNNDYETADGGYMTLNPRAPTDDDKNIYLTLPPNDHVNSNN (SEQ ID NO:222) |
| S076 | FCGRA2 transcript variant 1 NM_001136219_1 | CRKKRISANSTDPVKAAQFEPPGRQMIAIRKRQLEETNNDYETADGGYMTLNPRAPTDDDKNIYLTLPPNDHVNSNN (SEQ ID NO:223) |
| S077 | GHR transcript variant 1 NM_000163_4 | KQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP (SEQ ID NO:224) |
| S080 | ICOS NM_012092.3 | CWLTKKKYSSSVHDPNGEYMFMRAVNTAKKSRLTDVTL (SEQ ID NO:225) |
| S081 | IFNAR1 NM_000629_2 | KVFLRCINYVFFPSLKPSSSIDEYFSEQPLKNLLLSTSEEQIEKCFIIENISTIATVEETNQTDEDHKKYSSQTSQDSGNYSNEDESESKTSEELQQDFV (SEQ ID NO:226) |
| S082 | IFNAR2 transcript variant 1 NM_207585_2 | KWIGYICLRNSLPKVLNFHNFLAWPFPNLPPLEAMDMVEVIYINRKKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR (SEQ ID NO:227) |
| S083 | IFNAR2 transcript variant 2 NM_000874_4 | KWIGYICLRNSLPKVLRQGLAKGWNAVAIHRCSHNALQSETPELKQSSCLSFPSSWDYKRASLCPSD (SEQ ID NO:228) |
| S084 | IFNGR1 NM_000416_2 | CFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS (SEQ ID NO:229) |
| S085 | IFNGR2 transcript variant 1 NM_001329128_1 | LVLKYRGLIKYWFHTPPSIPLQIEEYLKDPTQPILEALDKDSSPKDDVWDSVSIISFPEKEQEDVLQTL (SEQ ID NO:230) |
| S086 | IFNLR1 NM_170743_3 | KTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR (SEQ ID NO:231) |
| S087 | IFNLR1 transcript variant 2 NM_173064_2 | KTLMGNPWFQRAKMPRALELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR (SEQ ID NO:232) |
| S098 | IL1R1 transcript variant 2 NM_001288706_1 | KIDIVLWYRDSCYDFLPIKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG (SEQ ID NO:233) |
| S099 | IL1R1 transcript variant 3 NM_001320978_1 | KIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKlQDYEKMPESIKFIKQKHGAIRWSGDFTQGPQSAKTRFWKNVRYHMPVQRRSPSSKHQLLSPATKEKLQREAHVPLG (SEQ ID NO:234) |
| S100 | IL1RAP transcript variant 1 NM_002182_3 | YRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV (SEQ ID NO:235) |
| S101 | IL1RAP transcript variant 6 NM_001167931_1 | YRAHFGTDETILDGKEYDIYVSYARNAEEEEFVLLTLRGVLENEFGYKLCIFDRDSLPGGNTVEAVFDFIQRSRRMIVVLSPDYVTEKSISMLEFKLGVMCQNSIATKLIVVEYRPLEHPHPGILQLKESVSFVSWKGEKSKHSGSKFWKALRLALPLRSLSASSGWNESCSSQSDISLDHVQRRRSRLKEPPELQSSERAAGSPPAPGTMSKHRGKSSATCRCCVTYCEGENHLRNKSRAEIHNQPQWETHLCKPVPQESETQWIQNGTRLEPPAPQISALALHHFTDLSNNNDFYIL (SEQ ID NO:236) |
| S102 | IL1RL1 transcript variant 1 NM_016232.4 | LKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ (SEQ ID NO:237) |
| S103 | IL1RL2 NM_003854.2 | NIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKH |
| | | GAIRWHGDFTEQSQCMKTKFWKTVRYHMPPRRCRPFPPVQLLQHTPCYRTAGPELGSRRKKCTLTTG (SEQ ID NO:238) |
| S104 | IL2RA transcript variant 1 NM_000417_2 | TWQRRQRKSRRTI (SEQ ID NO:239) |
| S105 | IL2RB transcript variant 1 NM_000878_4 | NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV (SEQ ID NO:240) |
| S106 | IL2RG NM_000206_2 | ERTMPRIPTLKNLEDLVTEYHGNFSAWSGVSKGLAESLQPDYSERLCLVSElPPKGGALGEGPGASPCNQHSPYWAPPCYTLKPET (SEQ ID NO:241) |
| S109 | IL3RA transcript variant 1 and 2 NM_002183_3 | RRYLVMQRLFPRIPHMKDPIGDSFQNDKLVVWEAGKAGLEECLVTEVQVVQKT (SEQ ID NO:242) |
| S110 | IL4R transcript variant 1 NM_000418_3 | KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPE KCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS (SEQ ID NO:243) |
| S113 | IL4R transcript variant 1 NM_000418_3 | KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPE KCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVFSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS (SEQ ID NO:244) |
| S115 | IL5RA transcript variant 1 NM_000564_4 | KICHLWIKLFPPIPAPKSNIKDLFVTTNYEKAGSSETEIEVICYIEKPGVETLEDSVF (SEQ ID NO:245) |
| S116 | IL6R transcript variant 1 NM 000565 3 | RFKKTWKLRALKEGKTSMHPPYSLGQLVPERPRPTPVLVPLISPPVSPSSLGSDNTSSHNRPDARDPRSPYDISNTDYFFPR (SEQ ID NO:246) |
| S117 | IL6ST transcript variant 1 and 3 NM_002184_3 | NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ (SEQ ID NO:247) |
| S120 | IL7RA Isoform 1 NM_002185.4 | WKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ (SEQ ID NO:248) |
| S121 | IL7RA Isoform 3 (C-term deletion) (interleukin 7 receptor) | WKKRIKPIVWPSLPDHKKTLEHLCKKPRKVSVFGA (SEQ ID NO:249) |
| S126 | IL9R transcript variant 1 NM_002186_2 | KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF (SEQ ID NO:250) |
| S129 | IL10RA transcript variant 1 NM_001558_3 | QLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE (SEQ ID NO:251) |
| S130 | IL10RB NM_000628_4 | ALLWCVYKKTKYAFSPRNSLPQHLKEFLGHPHHNTLLFFSFPLSDENDVFDKLSVIAEDSESGKQNPGDSCSLGTPPGQGPQS (SEQ ID NO:252) |
| S135 | IL11RA NM_001142784_2 | RLRRGGKDGSPKPGFLASVIPVDRRPGAPNL (SEQ ID NO:253) |
| S136 | IL12RB1 transcript variant 1 and 4 NM_005535_2 | NRAARHLCPPLPTPCASSAIEFPGGKETWQWINPVDFQEEASLQEALVVEMSWDKGERTEPLEKTELPEGAPELALDTELSLEDGDRCKAKM (SEQ ID NO:254) |
| S137 | IL12RB1 transcript variant 3 NM_001290023_1 | NRAARHLCPPLPTPCASSAIEFPGGKETWQWINPVDFQEEASLQEALVVEMSWDKGERTEPLEKTELPEGAPELALDTELSLEDGDRCDR (SEQ ID NO:255) |
| S138 | IL12RB2 transcript variant 1 and 3 NM_001559_2 | HYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML (SEQ ID NO:256) |
| S141 | IL13RA1 NM_001560_2 | KRLKIIIFPPIPDPGKIFKEMFGDQNDDTLHWKKYDIYEKQTKEETDSVVLIENLKKASQ (SEQ ID NO:257) |
| S142 | IL13RA2 NM_000640_2 | RKPNTYPKMIPEFFCDT (SEQ ID NO:258) |
| S143 | IL15RA transcript variant 4 NM_001256765_1 | KSRQTPPLASVEMEAMEALPVTWGTSSRDEDLENCSHHL (SEQ ID NO:259) |
| S144 | IL17RA NM_014339_6 | CMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA (SEQ ID NO:260) |
| S145 | IL17RB NM_018725_3 | RHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL (SEQ ID NO:261) |
| S146 | IL17RC transcript variant 1 NM_153460_3 | KKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT (SEQ ID NO:262) |
| S147 | IL17RC transcript variant 4 NM_001203263_1 | 3KKDHAKAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT (SEQ ID NO:263) |
| S148 | IL17RD transcript variant 2 NM_017563_4 | CRKKQQENIYSHLDEESSESSTYTAALPRERLRPRPKVFLCYSSKDGQNHMNVVQCFAYFLQDFCGCEVALDLWEDFSLCREGQREWVIQKIHESQFIIVVCSKGMKYFVDKKNYKHKGGGRGSGKGELFLVAVSAIAEKLRQAKQSSSAALSKFIAVYFDYSCEGDVPGILDLSTKYRLMDNLPQLCSHLHSRDHGLQEPGQHTRQGSRRNYFRSKSGRSLYVAICNMHQFIDEEPDWFEKQFVPFHPPPLRYREPVLEKFDSGLVLNDVMCKPGPESDFCLKVEAAVLGATGPADSQHESQHGGLDQDGEARPALDGSAALQPLLHTVKAGSPSDMPRDSGIYDSSVPSSELSLPLMEG LSTDQTETSSLTESVSSSSGLGEEEPPALPSKLLSSGSCKADLGCRSYTDELHAVAPL (SEQ ID NO:264) |
| S149 | IL17RE transcript variant 1 NM_153480_1 | TCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG (SEQ ID NO:265) |
| S154 | IL18R1 transcript variant 1 NM_003855_3 | YRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES (SEQ ID NO:266) |
| S155 | IL18RAP NM_003853_3 | SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW (SEQ ID NO:267) |
| S156 | IL20RA transcript variant 1 NM_014432_3 | SIYRYIHVGKEKHPANLILIYGNEFDKRFFVPAEKIVINFITLNISDDSKISHQDMSLLGKSSDVSSLNDPQPSGNLRPPQEEEEVKHLGYASHLMEIFCDSEENTEGTSLTQQESLSRTIPPDKTVIEYEYDVRTTDICAGPEEQELSLQEEVSTQGTLLESQAALAVLGPQTLQYSYTPQLQDLDPLAQEHTDSEEGPEEEPSTTLVDWDPQTGRLCIPSLSSFDQDSEGCEPSEGDGLGEEGLLSRLYEEPAPDRPPGENETYLMQFMEEWGLYVQMEN (SEQ ID NO:268) |
| S157 | IL20RB NM_144717_3 | WKMGRLLQYSCCPVVVLPDTLKITNSPQKLISCRREEVDACATAVMSPEELLRAWIS (SEQ ID NO:269) |
| S158 | IL21R transcript variant 2 NM_181078_2 | SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS (SEQ ID NO:270) |
| S161 | IL22RA1 NM_021258_3 | SYRYVTKPPAPPNSLNVQRVLTFQPLRFIQEHVLIPVFDLSGPSSLAQPVQYSQIRVSGPREPAGAPQRHSLSEITYLGQPDISILQPSNVPPPQILSPLSYAPNAAPEVGPPSYAPQVTPEAQFPFYAPQAISKVQPSSYAPQATPDSWPPSYGVCMEGSGKDSPTGTLSSPKHLRPKGQLQKEPPAGSCMLGGLSLQEVTSLAMEESQEAKSLHQPLGICTDRTSDPNVLHSGEEGTPQYLKGQLPLLSSVQIEGHPMSLPLQPPSRPCSPSDQGPSPWGLLESLVCPKDEAKSPAPETSDLEQPTELDSLFRGLALTVQWES (SEQ ID NO:271) |
| S165 | IL23R NM_144701_2 | NRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK (SEQ ID NO:272) |
| S168 | IL27RA NM_004843_3 | TSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA (SEQ ID NO:273) |
| S169 | IL27RA NM_004843_3 | TSWVWEKVPDPANSSSGQPHMEQVPEAQPLGDLPILEVEEMEPPPVMESSQPAQATAPLDSGYEKHFLPTPEELGLLGPPRPQVLA (SEQ ID NO:274) |
| S170 | IL31RA transcript variant 1 NM_139017_5 | KKPNKLTHLCWPTVPNPAESSIATWHGDDFKDKLNLKESDDSVNTEDRILKPCSTPSDKLVIDKLVVNFGNVLQEIFTDEARTGQENNLGGEKNGYVTCPFRPDCPLGKSFEELPVSPEIPPRKSQYLRSRMPEGTRPEAKEQLLFSGQSLVPDHLCEEGAPNPYLKNSVTAREFLVSEKLPEHTKGEV (SEQ ID NO:275) |
| S171 | IL31RA transcript variant 4 NM_001242638_1 | KKPNKLTHLCWPTVPNPAESSIATWHGDDFKDKLNLKESDDSVNTEDRILKPCSTPSDKLVIDKLVVNFGNVLQEIFTDEARTGQENNLGGEKNGTRILSSCPTSI (SEQ ID NO:276) |
| S174 | LEPR transcript variant 1 NM_002303_5 | SHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV (SEQ ID NO:277) |
| S175 | LEPR transcript variant 2 NM_001003680_3 | SHQRMKKLFWEDVPNPKNCSWAQGLNFQKMLEGSMFVKSHHHSLISSTQGHKHCGRPQGPLHRKTRDLCSLVYLLTLPPLLSYDPAKSPSVRNTQE (SEQ ID NO:278) |
| S176 | LEPR transcript variant 3 NM_001003679_3 | SHQRMKKLFWEDVPNPKNCSWAQGLNFQKRTDIL (SEQ ID NO:279) |
| S177 | LEPR transcript variant 5 NM_001198688_1 | SHQRMKKLFWEDVPNPKNCSWAQGLNFQKKMPGTKELLGGGWLT (SEQ ID NO:280) |
| S180 | LIFR NM_001127671_1 | YRKREWIKETFYPDIPNPENCKALQFQKSVCEGSSALKTLEMNPCTPNNVEVLETRSAFPKIEDTEIISPVAERPEDRSDAEPENHVVVSYCPPIIEEEIPNPAADEAGGTAQVIYIDVQSMYQPQAKPEEEQENDPVGGAGYKPQMHLPINSTVEDIAAEEDLDKTAGYRPQANVNTWNLVSPDSPRSIDSNSEIVSFGSPCSINSRQFLIPPKDEDSPKSNGGGWSFTNFFQNKPND (SEQ ID NO:281) |
| S183 | LMP1 NC_007605_1 | YYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD (SEQ ID NO:282) |
| S186 | MPL NM_005373_2 | RWQFPAHYRRLRHALWPSLPDLHRVLGQYLRDTAALSPPKATVYSDTCEEVEPSLLEILPKSSERTPLPLCSSQAQMDYRRLQPSCLGTMPLSVCPPMAESGSCCTTHIANHSYLPLSYWQQP (SEQ ID NO:283) |
| S189 | MYD88 transcript variant 1 NM_001172567_1 | MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRLARRPRGGCRRMVVVVSDDYLQSKECDFQTKFALSLSPGAHQKRLIPIKYKAMKKEFPSILRFITVCDYTNPCTKSWFWTRLAKALSLP (SEQ ID NO:284) |
| S190 | MYD88 transcript variant 2 NM_002468_4 | MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP (SEQ ID NO:285) |
| S191 | MYD88 transcript variant 3 NM_001172568_1 | MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP (SEQ ID NO:286) |
| S192 | MYD88 transcript variant 4 NM_001172569_1 | MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGAAGWWWLSLMITCRARNVTSRPNLHSASLQVPIRSD (SEQ ID NO:287) |
| S193 | MYD88 transcript variant 5 NM_001172566_1 | MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIGAAGWWWLSLMITCRARNVTSRPNLHSASLQVPIRSD (SEQ ID NO:288) |
| S194 | MYD88 transcript variant 1 NM 001172567 1 | MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI (SEQ ID NO:289) |
| S195 | MYD88 transcript variant 3 NM_001172568_1 | MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIGHMPERFDAFICYCPSDI (SEQ ID NO:290) |
| S196 | MYD88 transcript variant 1 NM_001172567_1 | MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRLARRPRGGCRRMVVVVSDDYLQSKECDFQTKFALSLSPGAHQKRPIPIKYKAMKKEFPSILRFITVCDYTNPCTKSWFWTRLAKALSLP (SEQ ID NO:291) |
| S197 | MYD88 transcript variant 2 NM_002468_4 | MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRR |
| | | MVVVVSDDYLQSKECDFQTKFALSLSPGAHQKRPIPIKYKAMKKEFPSILRFITVCDYTNPCTKSWFWTRLAKALSLP (SEQ ID NO:292) |
| S198 | MYD88 transcript variant 3 NM_001172568_1 | MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRPIPIKYKAMKKEFPSILRFITVCDYTNPCTKSWFWTRLAKALSLP (SEQ ID NO:293) |
| S199 | OSMR transcript variant 4 NM_001323505_1 | KSQWIKETCYPDIPDPYKSSILSLIKFKENPHLIIMNVSDCIPDAIEVVSKPEGTKIQFLGTRKSLTETELTKPNYLYLLPTEKNHSGPGPCICFENLTYNQAASDSGSCGHVPVSPKAPSMLGLMTSPENVLKALEKNYMNSLGEIPAGETSLNYVSQLASPMFGDKDSLPTNPVEAPHCSEYKMQMAVSLRLALPPPTENSSLSSITLLDPGEHYC (SEQ ID NO:294) |
| S202 | PRLR transcript variant 1 NM_000949_6 | KGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH (SEQ ID NO:295) |
| S211 | TNFRSF4 NM_003327_3 | ALYLLRRDQRLPPDAHKPPGGGSFRTPIQEEQADAHSTLAKI (SEQ ID NO:296) |
| S212 | TNFRSF8 transcript variant 1 NM_001243_4 | HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK (SEQ ID NO:297) |
| S213 | TNFRSF9 NM_001561_5 | KRGRKKLLYIFKQPFMRPVQTTQEEDGCSCRFPEEEEGGCEL (SEQ ID NO:298) |
| S214 | TNFRSF14 transcript variant 1 NM_003820_3 | CVKRRKPRGDVVKVIVSVQRKRQEAEGEATVIEALQAPPDVTTVAVEETIPSFTGRSPNH (SEQ ID NO:299) |
| S215 | TNFRSF18 transcript variant 1 NM_004195_2 | QLGLHIWQLRSQCMWPRETQLLLEVPPSTEDARSCQFPEEERGERSAEEKGRLGDLWV (SEQ ID NO:300) |
| S216 | TNFRSF18 transcript variant 3_NM_148902_1 | QLGLHIWQLRKTQLLLEVPPSTEDARSCQFPEEERGERSAEEKGRLGDLWV (SEQ ID NO:301) |
| X001 | Linker | GSGGSEGGGSEGGAATAGSGSGS (SEQ ID NO:302) |

TABLE 2

Ab1.1 Clones from 4^{th} panning round

| Ab1.1 Clone | Ab1.1 | Control | Ratio | Ab1.1 Clone | Ab1.1 | Control | Ratio |
|---|---|---|---|---|---|---|---|
| 1 | 2.4588 | 0.549 | 4.5 | 46 | 1.9639 | 0.6448 | 3.0 |
| 2 | 1.8091 | 0.4484 | 4.0 | 47 | 1.1639 | 0.7017 | 1.7 |
| 3 | 0.6065 | 0.5751 | 1.1 | 48 | 1.9877 | 0.6818 | 2.9 |
| 4 | 2.2638 | 0.6021 | 3.8 | 49 | 1.1885 | 0.6323 | 1.9 |
| 5 | 0.6107 | 0.6563 | 0.9 | 50 | 1.3607 | 0.6415 | 2.1 |
| 6 | 1.3868 | 0.4654 | 3.0 | 51 | 1.1857 | 0.6656 | 1.8 |
| 7 | 1.7038 | 0.5912 | 2.9 | 52 | 1.4835 | 0.6945 | 2.1 |
| 8 | 1.8222 | 0.5429 | 3.4 | 53 | 1.7942 | 0.6871 | 2.6 |
| 9 | 1.8769 | 0.6839 | 2.7 | 54 | 1.2299 | 0.6208 | 2.0 |
| 10 | 1.4259 | 0.4908 | 2.9 | 55 | 1.9349 | 0.6492 | 3.0 |
| 11 | 1.6424 | 0.5697 | 2.9 | 56 | 1.7907 | 0.6718 | 2.7 |
| 12 | 1.5823 | 0.558 | 2.8 | 57 | 1.0701 | 0.6496 | 1.6 |
| 13 | 0.3612 | 0.5903 | 0.6 | 58 | 1.381 | 0.6183 | 2.2 |
| 14 | 0.9557 | 0.463 | 2.1 | 59 | 1.8883 | 0.6622 | 2.9 |
| 15 | 1.5047 | 0.5233 | 2.9 | 60 | 2.2758 | 0.6511 | 3.5 |
| 16 | 1.0152 | 0.5695 | 1.8 | 61 | 2.2687 | 0.6463 | 3.5 |
| 17 | 0.4691 | 0.6741 | 0.7 | 62 | 1.8997 | 0.6912 | 2.7 |
| 18 | 0.7718 | 0.4646 | 1.7 | 63 | 1.7105 | 0.6386 | 2.7 |
| 19 | 1.7226 | 0.54 | 3.2 | 64 | 1.8005 | 0.655 | 2.7 |
| 20 | 0.6258 | 0.563 | 1.1 | 65 | 2.2723 | 0.6223 | 3.7 |
| 21 | 1.0541 | 0.7099 | 1.5 | 66 | 1.2177 | 0.7047 | 1.7 |
| 22 | 1.7285 | 0.485 | 3.6 | 67 | 1.6295 | 0.7126 | 2.3 |
| 23 | 1.122 | 0.6174 | 1.8 | 68 | 1.5747 | 0.6826 | 2.3 |
| 24 | 1.6164 | 0.5771 | 2.8 | 69 | 1.3397 | 0.6129 | 2.2 |
| 25 | 0.3558 | 0.5334 | 0.7 | 70 | 1.7763 | 0.6535 | 2.7 |
| 26 | 0.2689 | 0.6977 | 0.4 | 71 | 1.7183 | 0.6057 | 2.8 |
| 27 | 0.5302 | 0.9309 | 0.6 | 72 | 1.2671 | 0.6081 | 2.1 |
| 28 | 0.3543 | 0.6183 | 0.6 | 73 | 1.67 | 1.1257 | 1.5 |
| 29 | 0.2265 | 0.5698 | 0.4 | 74 | 1.4253 | 0.6145 | 2.3 |
| 30 | 0.2102 | 0.6212 | 0.3 | 75 | 1.0627 | 0.5645 | 1.9 |
| 31 | 0.2115 | 0.5947 | 0.4 | 76 | 1.5179 | 0.5693 | 2.7 |
| 32 | 0.2662 | 0.6027 | 0.4 | 77 | 1.5533 | 0.5835 | 2.7 |
| 33 | 0.3696 | 0.5669 | 0.7 | 78 | 1.6402 | 0.5382 | 3.0 |
| 34 | 0.2246 | 0.6243 | 0.4 | 79 | 2.0145 | 0.617 | 3.3 |
| 35 | 0.2081 | 0.6078 | 0.3 | 80 | 1.7805 | 0.5014 | 3.6 |
| 36 | 0.2976 | 0.6931 | 0.4 | 81 | 1.6182 | 0.4758 | 3.4 |
| 37 | 0.9743 | 0.5667 | 1.7 | 82 | 1.7224 | 0.4733 | 3.6 |
| 38 | 0.2064 | 0.5734 | 0.4 | 83 | 2.1371 | 0.5561 | 3.8 |
| 39 | 0.195 | 0.5709 | 0.3 | 84 | 1.6939 | 0.5621 | 3.0 |
| 40 | 0.2767 | 0.662 | 0.4 | 85 | 2.196 | 0.5364 | 4.1 |
| 41 | 0.2598 | 0.687 | 0.4 | 86 | 2.3053 | 0.5435 | 4.2 |
| 42 | 0.2445 | 0.6656 | 0.4 | 87 | 1.9764 | 0.5398 | 3.7 |
| 43 | 0.1862 | 0.5817 | 0.3 | 88 | 1.3631 | 0.5188 | 2.6 |
| 44 | 0.2511 | 0.6399 | 0.4 | 89 | 2.0549 | 0.594 | 3.5 |
| 45 | 1.9493 | 0.6184 | 3.2 | | | | |

TABLE 3

Ab1.1 Clones from 5^{th} panning round

| Ab1.1 Clone | Ab1.1 | Control | Ratio | Ab1.1 Clone | Ab1.1 | Control | Ratio |
|---|---|---|---|---|---|---|---|
| 90 | 1.4856 | 0.6047 | 2.5 | 186 | 0.1794 | 0.1382 | 1.3 |
| 91 | 1.6268 | 0.4791 | 3.4 | 187 | 0.4721 | 0.1251 | 3.8 |
| 92 | 1.2074 | 0.48 | 2.5 | 188 | 0.4024 | 0.1096 | 3.7 |
| 93 | 0.8499 | 0.4531 | 1.9 | 189 | 0.1828 | 0.1246 | 1.5 |
| 94 | 1.5283 | 0.4411 | 3.5 | 190 | 0.4845 | 0.1147 | 4.2 |
| 95 | 1.3589 | 0.4972 | 2.7 | 191 | 0.2009 | 0.1469 | 1.4 |
| 96 | 1.835 | 0.4904 | 3.7 | 192 | 0.0724 | 0.1304 | 0.6 |
| 97 | 1.418 | 0.4873 | 2.9 | 193 | 0.5504 | 0.1322 | 4.2 |
| 98 | 1.4241 | 0.4838 | 2.9 | 194 | 0.1747 | 0.1292 | 1.4 |
| 99 | 0.9938 | 0.5013 | 2.0 | 195 | 0.1918 | 0.1267 | 1.5 |
| 100 | 0.7618 | 0.4606 | 1.7 | 196 | 0.4586 | 0.1217 | 3.8 |
| 101 | 1.2826 | 0.4897 | 2.6 | 197 | 0.2008 | 0.1623 | 1.2 |
| 102 | 1.5341 | 0.4894 | 3.1 | 198 | 0.0794 | 0.1042 | 0.8 |
| 103 | 0.8302 | 0.5143 | 1.6 | 199 | 0.1386 | 0.1025 | 1.4 |
| 104 | 0.4899 | 0.463 | 1.1 | 200 | 0.1566 | 0.1095 | 1.4 |
| 105 | 1.164 | 0.4827 | 2.4 | 201 | 0.3392 | 0.1052 | 3.2 |
| 106 | 1.1465 | 0.4609 | 2.5 | 202 | 0.1697 | 0.1128 | 1.5 |
| 107 | 0.5959 | 0.4593 | 1.3 | 203 | 0.2718 | 0.1108 | 2.5 |
| 108 | 1.1027 | 0.6966 | 1.6 | 204 | 0.1032 | 0.1256 | 0.8 |
| 109 | 1.3299 | 0.4855 | 2.7 | 205 | 0.0841 | 0.117 | 0.7 |
| 110 | 0.9404 | 0.4794 | 2.0 | 206 | 0.3912 | 0.105 | 3.7 |
| 111 | 0.8598 | 0.4569 | 1.9 | 207 | 0.3123 | 0.2322 | 1.3 |
| 112 | 0.4028 | 0.4425 | 0.9 | 208 | 0.1386 | 0.1308 | 1.1 |
| 113 | 0.2716 | 0.5142 | 0.5 | 209 | 0.1522 | 0.1431 | 1.1 |
| 114 | 0.1792 | 0.459 | 0.4 | 210 | 0.0707 | 0.0882 | 0.8 |
| 115 | 0.2675 | 0.6797 | 0.4 | 211 | 0.1675 | 0.1278 | 1.3 |
| 116 | 0.164 | 0.4277 | 0.4 | 212 | 0.1705 | 0.1084 | 1.6 |
| 117 | 0.4299 | 0.4452 | 1.0 | 213 | 0.2875 | 0.1062 | 2.7 |
| 118 | 0.267 | 0.458 | 0.6 | 214 | 0.3101 | 0.2086 | 1.5 |
| 119 | 0.2291 | 0.4601 | 0.5 | 215 | 0.3351 | 0.0937 | 3.6 |
| 120 | 0.1834 | 0.4759 | 0.4 | 216 | 0.3888 | 0.0974 | 4.0 |
| 121 | 0.4776 | 0.4246 | 1.1 | 217 | 0.2089 | 0.1025 | 2.0 |
| 122 | 0.6578 | 0.4225 | 1.6 | 218 | 0.11 | 0.0903 | 1.2 |
| 123 | 0.5838 | 0.459 | 1.3 | 219 | 0.1329 | 0.1169 | 1.1 |
| 124 | 0.2812 | 0.4281 | 0.7 | 220 | 0.48 | 0.1173 | 4.1 |
| 125 | 1.0784 | 0.4481 | 2.4 | 221 | 0.3417 | 0.1173 | 2.9 |
| 126 | 0.8106 | 0.5288 | 1.5 | 222 | 0.2496 | 0.0967 | 2.6 |
| 127 | 1.2029 | 0.4201 | 2.9 | 223 | 0.2632 | 0.1089 | 2.4 |
| 128 | 0.2542 | 0.4606 | 0.6 | 224 | 0.2447 | 0.1026 | 2.4 |
| 129 | 1.3 | 0.4595 | 2.8 | 225 | 0.3436 | 0.106 | 3.2 |
| 130 | 0.2083 | 0.4903 | 0.4 | 226 | 0.103 | 0.1223 | 0.8 |
| 131 | 1.4514 | 0.4162 | 3.5 | 227 | 0.3274 | 0.0956 | 3.4 |
| 132 | 0.5408 | 0.4717 | 1.1 | 228 | 0.303 | 0.1011 | 3.0 |
| 133 | 0.4076 | 0.439 | 0.9 | 229 | 0.1799 | 0.0933 | 1.9 |
| 134 | 0.9592 | 0.5445 | 1.8 | 230 | 0.2065 | 0.1067 | 1.9 |
| 135 | 0.9574 | 0.4188 | 2.3 | 231 | 0.0928 | 0.1271 | 0.7 |
| 136 | 0.7604 | 0.4226 | 1.8 | 232 | 0.4444 | 0.1225 | 3.6 |
| 137 | 0.5895 | 0.553 | 1.1 | 233 | 0.0977 | 0.1166 | 0.8 |
| 138 | 0.5465 | 0.4183 | 1.3 | 234 | 0.0973 | 0.1044 | 0.9 |
| 139 | 0.7304 | 0.4267 | 1.7 | 235 | 0.2504 | 0.1073 | 2.3 |
| 140 | 1.0639 | 0.3941 | 2.7 | 236 | 0.2878 | 0.1011 | 2.8 |
| 141 | 1.0286 | 0.3993 | 2.6 | 237 | 0.2485 | 0.1182 | 2.1 |
| 142 | 0.9285 | 0.3881 | 2.4 | 238 | 0.246 | 0.1109 | 2.2 |
| 143 | 1.0762 | 0.4204 | 2.6 | 239 | 0.2908 | 0.0907 | 3.2 |
| 144 | 0.1564 | 0.5332 | 0.3 | 240 | 0.305 | 0.0941 | 3.2 |
| 145 | 0.7024 | 0.4304 | 1.6 | 241 | 0.2545 | 0.108 | 2.4 |
| 146 | 0.8023 | 0.4583 | 1.8 | 242 | 0.298 | 0.1021 | 2.9 |
| 147 | 0.4551 | 0.4441 | 1.0 | 243 | 0.2792 | 0.1068 | 2.6 |
| 148 | 0.7863 | 0.4292 | 1.8 | 244 | 0.3429 | 0.1016 | 3.4 |
| 149 | 1.0984 | 0.3936 | 2.8 | 245 | 0.0685 | 0.1127 | 0.6 |
| 150 | 0.766 | 0.5142 | 1.5 | 246 | 0.5484 | 0.1027 | 5.3 |
| 151 | 0.8354 | 0.4366 | 1.9 | 247 | 0.4127 | 0.1294 | 3.2 |
| 152 | 0.8318 | 0.4614 | 1.8 | 248 | 0.2673 | 0.1067 | 2.5 |
| 153 | 0.8058 | 0.4345 | 1.9 | 249 | 0.2814 | 0.1099 | 2.6 |
| 154 | 0.9087 | 0.4574 | 2.0 | 250 | 0.0591 | 0.1379 | 0.4 |
| 155 | 1.6017 | 0.4168 | 3.8 | 251 | 0.117 | 0.0998 | 1.2 |
| 156 | 0.7758 | 0.5313 | 1.5 | 252 | 0.191 | 0.102 | 1.9 |
| 157 | 0.1333 | 0.4229 | 0.3 | 253 | 0.129 | 0.102 | 1.3 |
| 158 | 0.1407 | 0.451 | 0.3 | 254 | 0.2497 | 0.1007 | 2.5 |
| 159 | 0.6762 | 0.4166 | 1.6 | 255 | 0.4235 | 0.1347 | 3.1 |
| 160 | 0.1419 | 0.454 | 0.3 | 256 | 0.3783 | 0.116 | 3.3 |
| 161 | 1.4207 | 0.4471 | 3.2 | 257 | 0.315 | 0.1254 | 2.5 |
| 162 | 0.1497 | 0.538 | 0.3 | 258 | 0.0876 | 0.1188 | 0.7 |
| 163 | 0.1454 | 0.4756 | 0.3 | 259 | 0.34 | 0.1468 | 2.3 |
| 164 | 0.1473 | 0.4649 | 0.3 | 260 | 0.4264 | 0.1123 | 3.8 |
| 165 | 0.1514 | 0.4377 | 0.3 | 261 | 0.1613 | 0.1222 | 1.3 |
| 166 | 0.2845 | 0.4471 | 0.6 | 262 | 0.1196 | 0.0655 | 1.8 |
| 167 | 0.2171 | 0.4344 | 0.5 | 263 | 0.145 | 0.1012 | 1.4 |
| 168 | 0.5434 | 0.5374 | 1.0 | 264 | 0.1993 | 0.1084 | 1.8 |
| 169 | 0.9217 | 0.4211 | 2.2 | 265 | 0.4167 | 0.0935 | 4.5 |
| 170 | 0.1453 | 0.435 | 0.3 | 266 | 0.3363 | 0.1434 | 2.3 |
| 171 | 0.1758 | 0.4331 | 0.4 | 267 | 0.3431 | 0.1139 | 3.0 |
| 172 | 0.3842 | 0.4169 | 0.9 | 268 | 0.2854 | 0.1208 | 2.4 |
| 173 | 0.36 | 0.4249 | 0.8 | 269 | 0.5569 | 0.1151 | 4.8 |
| 174 | 0.9269 | 0.4152 | 2.2 | 270 | 0.6375 | 0.1486 | 4.3 |
| 175 | 0.155 | 0.4168 | 0.4 | 271 | 0.2458 | 0.1352 | 1.8 |
| 176 | 0.7722 | 0.4361 | 1.8 | 272 | 0.2989 | 0.1283 | 2.3 |
| 177 | 0.2203 | 0.3929 | 0.6 | 273 | 0.4145 | 0.1386 | 3.0 |
| 178 | 0.8812 | 0.3958 | 2.2 | 274 | 0.5047 | 0.1433 | 3.5 |
| 179 | 0.7869 | 0.4463 | 1.8 | 275 | 0.3222 | 0.1301 | 2.5 |
| 180 | 1.0636 | 0.426 | 2.5 | 276 | 0.5643 | 0.1792 | 3.1 |
| 181 | 0.1641 | 0.4658 | 0.4 | 277 | 0.4805 | 0.1289 | 3.7 |
| 182 | 0.5162 | 0.4344 | 1.2 | 278 | 0.4756 | 0.1232 | 3.9 |
| 183 | 0.5199 | 0.4504 | 1.2 | 279 | 0.2675 | 0.1237 | 2.2 |
| 184 | 0.6984 | 0.4511 | 1.5 | 280 | 0.4527 | 0.1278 | 3.5 |
| 185 | 0.998 | 0.5748 | 1.7 | 281 | 0.6807 | 0.1608 | 4.2 |

TABLE 4

Ab1.2 Clones from 2^{nd} panning round

| Ab1.2 Clone | Ab1.2 | Control | Ratio | Ab1.2 Clone | Ab1.2 | Control | Ratio |
|---|---|---|---|---|---|---|---|
| 1 | 0.79 | 0.30 | 2.6 | 16 | 1.08 | 0.12 | 9.1 |
| 2 | 1.00 | 0.14 | 7.0 | 17 | 1.20 | 0.31 | 3.8 |
| 3 | 1.14 | 0.31 | 3.7 | 18 | 0.97 | 0.11 | 9.1 |
| 4 | 1.07 | 0.47 | 2.3 | 19 | 1.08 | 0.08 | 13.6 |
| 5 | 1.05 | 0.10 | 10.2 | 20 | 0.87 | 0.08 | 10.6 |
| 6 | 1.12 | 0.14 | 8.2 | 21 | 0.71 | 0.07 | 9.9 |
| 7 | 0.85 | 0.11 | 7.6 | 22 | 0.79 | 0.06 | 12.3 |
| 8 | 0.80 | 0.18 | 4.5 | 23 | 0.75 | 0.36 | 2.1 |
| 9 | 0.87 | 0.09 | 9.5 | 24 | 0.34 | 0.06 | 5.6 |
| 10 | 0.20 | 0.05 | 3.9 | 25 | 1.23 | 0.67 | 1.8 |
| 11 | 1.03 | 0.05 | 18.9 | 26 | 1.23 | 0.61 | 2.0 |
| 12 | 1.34 | 0.21 | 6.2 | 27 | 1.06 | 0.36 | 2.9 |
| 13 | 0.15 | 0.10 | 1.5 | 28 | 0.73 | 0.24 | 3.0 |
| 14 | 1.09 | 0.09 | 12.0 | 29 | 1.17 | 0.12 | 9.5 |
| 15 | 0.90 | 0.07 | 12.5 | 30 | 0.72 | 0.16 | 4.6 |

TABLE 5

Ab1.2 Clones from 3^{rd} panning round

| Ab1.2 Clone | Ab1.2 | Control | Ratio | Ab1.2 Clone | Ab1.2 | Control | Ratio |
|---|---|---|---|---|---|---|---|
| 31 | 0.73 | 0.15 | 5.0 | 64 | 1.11 | 0.10 | 11.3 |
| 32 | 0.91 | 0.11 | 8.6 | 65 | 0.59 | 0.09 | 6.8 |
| 33 | 1.05 | 0.17 | 6.3 | 66 | 0.64 | 0.05 | 12.3 |
| 34 | 0.84 | 0.32 | 2.7 | 67 | 0.71 | 0.12 | 5.8 |
| 35 | 1.01 | 0.18 | 5.8 | 68 | 1.15 | 0.06 | 17.6 |
| 36 | 0.40 | 0.11 | 3.8 | 69 | 1.01 | 0.09 | 11.5 |
| 37 | 1.07 | 0.12 | 8.6 | 70 | 1.01 | 0.06 | 15.9 |
| 38 | 1.07 | 0.08 | 12.7 | 71 | 1.08 | 0.06 | 18.0 |
| 39 | 0.75 | 0.13 | 5.9 | 72 | 0.54 | 0.08 | 6.5 |
| 40 | 1.08 | 0.08 | 13.1 | 73 | 0.87 | 0.09 | 9.5 |
| 41 | 1.01 | 0.07 | 13.7 | 74 | 1.11 | 0.14 | 8.1 |
| 42 | 0.71 | 0.11 | 6.7 | 75 | 1.16 | 0.32 | 3.6 |
| 43 | 1.09 | 0.09 | 12.0 | 76 | 0.40 | 0.26 | 1.5 |
| 44 | 0.79 | 0.11 | 6.9 | 77 | 1.06 | 0.10 | 10.6 |
| 45 | 1.10 | 0.46 | 2.4 | 78 | 0.78 | 0.06 | 12.6 |
| 46 | 1.00 | 0.28 | 3.5 | 79 | 1.05 | 0.89 | 1.2 |
| 47 | 0.71 | 0.21 | 3.4 | 80 | 0.62 | 0.05 | 13.0 |
| 48 | 1.08 | 0.32 | 3.3 | 81 | 0.87 | 0.16 | 5.5 |
| 49 | 1.18 | 0.31 | 3.8 | 82 | 0.53 | 0.42 | 1.3 |
| 50 | 0.69 | 0.41 | 1.7 | 83 | 0.77 | 0.10 | 7.8 |
| 51 | 0.75 | 0.71 | 1.1 | 84 | 1.10 | 0.07 | 16.7 |
| 52 | 0.84 | 0.41 | 2.1 | 85 | 0.90 | 0.07 | 12.3 |
| 53 | 1.06 | 0.09 | 11.6 | 86 | 1.07 | 0.06 | 18.2 |
| 54 | 1.11 | 0.24 | 4.5 | 87 | 1.11 | 0.10 | 11.7 |
| 55 | 0.72 | 0.20 | 3.6 | 88 | 1.08 | 0.08 | 14.4 |
| 56 | 0.80 | 0.26 | 3.1 | 89 | 1.18 | 0.21 | 5.6 |
| 57 | 1.10 | 0.19 | 5.7 | 90 | 1.11 | 0.07 | 16.6 |
| 58 | 0.36 | 0.36 | 1.0 | 91 | 1.11 | 0.09 | 12.5 |
| 59 | 1.13 | 0.63 | 1.8 | 92 | 1.13 | 0.51 | 2.2 |
| 60 | 0.18 | 0.05 | 3.7 | 93 | 1.03 | 0.20 | 5.2 |
| 61 | 0.55 | 0.06 | 8.9 | 94 | 0.98 | 0.08 | 12.1 |
| 62 | 0.43 | 0.12 | 3.5 | 95 | 0.58 | 0.07 | 8.7 |
| 63 | 1.05 | 0.09 | 12.1 | | | | |

What is claimed is:

1. A polynucleotide, comprising: one or more transcriptional units, wherein each of the one or more transcriptional units is operatively linked to a promoter active in T cells and/or NK cells, wherein the one or more transcriptional units comprise:
a) nucleic acids encoding a first engineered signaling polypeptide, wherein the first engineered signaling polypeptide is a chimeric antigen receptor (CAR), and
b) nucleic acids encoding an anti-idiotype polypeptide comprising an anti-idiotype extracellular recognition domain, a membrane association domain, and a stalk that connects the anti-idiotype extracellular recognition domain to the membrane association domain, wherein the anti-idiotype extracellular recognition domain comprises an idiotype-binding variable region of an anti-idiotype antibody or antibody mimetic that recognizes the idiotype of a target antibody or a target antibody mimetic.

2. A modified mammalian cell, comprising the polynucleotide of claim 1.

3. A method for delivering modified T cells and/or NK cells to a subject, wherein the method comprises administering the modified T cells and/or NK cells to the subject, wherein the modified T cells and/or NK cells comprise the polynucleotide of claim 1.

4. Use of modified T cells and/or NK cells in the manufacture of a kit, wherein the use of the kit comprises: administering the modified T cells and/or NK cells to a subject, wherein the modified T cells and/or NK cells comprise the polynucleotide of claim 1.

5. A polynucleotide vector, comprising the polynucleotide of claim 1.

6. A polypeptide, comprising an extracellular recognition domain, a membrane association domain, and a stalk connecting the extracellular recognition domain to the membrane association domain, and further comprising an intracellular domain (ICD) of a first engineered signaling polypeptide, wherein the first engineered signaling polypeptide is a chimeric antigen receptor (CAR), a recombinant T cell receptor (TCR), or a lymphoproliferative element (LE), and wherein the extracellular recognition domain is an anti-idiotype extracellular recognition domain that recognizes an idiotype of a target antibody.

7. The polynucleotide of claim 1, the cell of claim 2, the method of claim 3, the use of claim 4, the polynucleotide vector of claim 5, or the polypeptide of claim 6, wherein the anti-idiotype extracellular recognition domain is an antibody comprising an scFv.

8. The polynucleotide of claim 1, the cell of claim 2, the method of claim 3, the use of claim 4, the polynucleotide vector of claim 5, or the polypeptide of claim 6, wherein the idiotype-binding variable region comprises a framework region, and wherein the framework region is a human framework region.

9. The modified mammalian cell of claim 2, wherein the cell is one of a population of human cells within an infusion bag.

10. The polynucleotide of claim 1, the cell of claim 2, the method of claim 3, the use of claim 4, or the polynucleotide vector of claim 5, wherein the polynucleotide further encodes a second engineered signaling polypeptide, and wherein the second engineered signaling polypeptide is a lymphoproliferative element.

11. The polynucleotide, the cell, the method, the use, the polynucleotide vector, or the polypeptide of claim 10, wherein the lymphoproliferative element comprises an intracellular domain that is a means for transmitting a signal that promotes proliferation or survival of a T cell and/or NK cell.

12. The polynucleotide, the cell, the method, the use, the polynucleotide vector, or the polypeptide of claim 11, wherein the lymphoproliferative element is constitutively active.

13. The polynucleotide, the cell, the method, the use, or the polynucleotide vector of claim 10, wherein the polynucleotide comprises an internal ribosome entry site (IRES), a ribosomal skip sequence and/or cleavage signal, between nucleic acids encoding the first engineered signaling polypeptide or the second engineered signaling polypeptide, and nucleic acids encoding the anti-idiotype polypeptide.

14. The polynucleotide, the cell, the method, the use, or the polynucleotide vector of claim 10, wherein the polynucleotide comprises an internal ribosome entry site (IRES), a ribosomal skip sequence and/or cleavage signal, between nucleic acids encoding the second engineered signaling polypeptide, and nucleic acids encoding the anti-idiotype polypeptide.

15. The polynucleotide of claim 1, the cell of claim 2, the method of claim 3, the use of claim 4, the polynucleotide vector of claim 5, or the polypeptide of claim 6, wherein the target antibody comprises an antigen-binding site, and wherein the anti-idiotype polypeptide is capable of binding to the antigen-binding site of the target antibody.

16. The polynucleotide, the cell, the method, the use, the polynucleotide vector, or the polypeptide of claim 15, wherein the target antibody or target antibody mimetic is an approved biologic antibody or antibody mimetic, approved by the Food And Drug Administration of the U.S. (USFDA), European Medicines Agency (EMA), National Medical Products Administration of China (NMPA) (Chinese FDA), or the Pharmaceutical and Food Safety Bureau (PFSB) of Japan.

17. The polynucleotide, the cell, the method, the use, the polynucleotide vector, or the polypeptide of claim 16, wherein the approved biologic antibody or antibody mimetic is the approved biologic antibody, and wherein the approved biologic antibody is cetuximab, muromonab-CD3, efalizumab, tositumomab-i131, nebacumab, edrecolomab, catumaxomab, daclizumab, olaratumab, abciximab, rituximab, basiliximab, palivizumab, infliximab, trastuzumab, adalimumab, ibritumomab tiuxetan, omalizumab, bevacizumab, natalizumab, panitumumab, ranibizumab, eculizumab, certolizumab pegol, ustekinumab, canakinumab, golimumab, ofatumumab, tocilizumab, denosumab, belimumab, ipilimumab, brentuximab vedotin, pertuzumab, ado-trastuzumab emtansine, raxibacumab, obinutuzumab, siltuximab, ramucirumab, vedolizumab, nivolumab, pembrolizumab, blinatumomab, alemtuzumab, evolocumab, idarucizumab, necitumumab, dinutuximab, secukinumab, mepolizumab, alirocumab, daratumumab, elotuzumab, ixekizumab, reslizumab, bezlotoxumab, atezolizumab, obiltoxaximab, brodalumab, dupilumab, inotuzumab ozogamicin, guselkumab, sarilumab, avelumab, emicizumab, ocrelizumab, benralizumab, durvalumab, gemtuzumab ozogamicin, erenumab (erenumab-aooe), galcanezumab (galcanezumab-gnlm), burosumab (burosumab-twza), lanadelumab (lanadelumab-flyo), mogamulizumab (mogamulizumab-kpkc), tildrakizumab (tildrakizumab-asmn), fremanezumab (fremanezumab-vfrm), ravulizumab (ravulizumab-cwvz), cemiplimab (cemiplimab-rwlc), ibalizumab (ibalizumab-uiyk) emapalumab (emapalumab-lzsg), moxetumomab pasudotox (moxetumomab pasudotox-tdfk), caplacizumab (caplacizumab-yhdp), risankizumab (risankizumab-rzaa), polatuzumab vedotin (polatuzumab vedotin-piiq), romosozumab (romosozumab-aqqg), brolucizumab (brolucizumab-dbll), crizanlizumab (crizanlizumab-tmca), enfortumab vedotin (enfortumab vedotin-ejfv), [fam-]trastuzumab deruxtecan (fam-trastuzumab deruxtecan-nxki), teprotumumab (teprotumumab-trbw), eptinezumab (eptinezumab-jjmr), isatuximab (isatuximab-irfc), sacituzumab govitecan (sacituzumab govitecan-hziy), inebilizumab (inebilizumab-cdon), tafasitamab (tafasitamab-cxix), belantamab mafodotin (belantamab mafodotin-blmf), satralizumab (satralizumab-mwge), atoltivimab, maftivimab, odesivimab-ebgn, naxitamab-gqgk, margetuximab-cmkb, ansuvimab-zykl, evinacumab, dostarlimab (dostarlimab-gxly), loncastuximab tesirine (loncastuximab tesirine-lpyl), amivantamab (amivantamab-vmjw), aducanumab (aducanumab-avwa), tralokinumab, anifrolumab (anifrolumab-fnia), oportuzumab monatox, tisotumab vedotin, bimekizumab, narsoplimab, tezepelumab, sintilimab, inolimomb, balstilimab, ublituximab, toripalimab, omburtamab, penpulimab, tanezumab, faricimab, sutimlimab, teplizumab, and retifanlimab.

18. The polynucleotide, the cell, the method, the use, the polynucleotide vector, or the polypeptide of claim 16, wherein the anti-idiotype polypeptide comprises a means for binding an idiotype of cetuximab.

19. The polynucleotide, the cell, the method, the use, the polynucleotide vector, or the polypeptide of claim 17, wherein the approved biologic antibody is cetuximab.

20. The polynucleotide, the cell, the method, the use, the polynucleotide vector, or the polypeptide of claim 19, wherein the anti-idiotype polypeptide is capable of blocking binding of cetuximab to epidermal growth factor receptor.

21. The polynucleotide of claim 1, the cell of claim 2, the method of claim 3, the use of claim 4, or the polynucleotide vector of claim 5, wherein the anti-idiotype polypeptide further comprises one or more intracellular domains (ICD).

22. The polynucleotide, the cell, the method, the use, the polynucleotide vector, or the polypeptide of claim 21, wherein the ICD is between 1 and 35 amino acids in length.

23. The polynucleotide, the cell, the method, the use, the polynucleotide vector of claim 21, or the polypeptide of claim 6, wherein the membrane association domain is a transmembrane domain.

24. The polynucleotide, the cell, the method, the use, the polynucleotide vector, or the polypeptide of claim 23, wherein the ICD comprises all or part of an intracellular signaling domain of one or more lymphoproliferative elements, and wherein the ICD is capable of activating a signaling pathway.

25. The polynucleotide, the cell, the method, the use, the polynucleotide vector, or the polypeptide of claim 24, wherein the lymphoproliferative element is inducible, and wherein the inducible lymphoproliferative element is capable of activating a proliferative or survival signaling after binding of the anti-idiotype polypeptide to the target antibody or target antibody mimetic.

26. The polynucleotide, the cell, the method, the use, the polynucleotide vector, or the polypeptide of claim 25, wherein the ICD is capable upon dimerization, of activating a Jak pathway, a TRAF pathway, a PI3K pathway, and/or a PLC pathway.

27. The polynucleotide, the cell, the method, the use, the polynucleotide vector, or the polypeptide of claim 25, wherein the idiotype is present on two target antibodies, a first target antibody, which promotes cytotoxicity, and a second target antibody, which promote less cytotoxicity than the first target antibody.

28. The polynucleotide, the cell, the method, the use, the polynucleotide vector, or the polypeptide of claim 24, wherein the one or more cytokine receptors are selected from CD27, CD40, CRLF2, CSF2RA, CSF2RB, CSF3R, EPOR, GHR, IFNAR1, IFNAR2, IFNGR1, IFNGR2, IFNLR1, IL1R1, IL1RAP, IL1RL1, IL1RL2, IL2R, IL2RA, IL2RB, IL2RG, IL3RA, IL4R, IL5RA, IL6R, IL6ST, IL7R, IL7RA, IL9R, IL10RA, IL10RB, IL11RA, IL12RB1, IL13R, IL13RA1, IL13RA2, IL15R, IL15RA, IL17RA, IL17RB, IL17RC, IL17RE, IL18R1, IL18RAP, IL20RA, IL20RB, IL21R, IL22RA1, IL23R, IL27R, IL27RA, IL31RA, LEPR, LIFR, MPL, OSMR, PRLR, TGFβR, TGFβ decoy receptor, TNFRSF4, TNFRSF8, TNFRSF9, TNFRSF14, or TNFRSF18.

29. The polynucleotide, the cell, the method, the use, the polynucleotide vector, or the polypeptide of claim 23, wherein the membrane association domain is the transmembrane domain of the lymphoproliferative element, the CAR, or the recombinant TCR.

30. The polynucleotide of claim 1, the cell of claim 2, the method of claim 3, the use of claim 4, the polynucleotide vector of claim 5, or the polypeptide of claim 6, wherein the stalk is between 4 and 100 amino acids in length.

31. The polynucleotide, the cell, the method, the use, the polynucleotide vector, or the polypeptide of claim 30, wherein the stalk encodes a dimerizing moiety, wherein the dimerizing moiety is constitutively dimerized.

32. The polynucleotide, the cell, the method, the use, the polynucleotide vector, or the polypeptide of claim 31, wherein the constitutively dimerized dimerization domain comprises a leucine zipper.

33. The polynucleotide of claim 1, the cell of claim 2, the method of claim 3, the use of claim 4, or the polynucleotide vector of claim 5, wherein the polynucleotide further comprises nucleic acids that encode one or more inhibitory RNA molecules.

34. The polynucleotide, the cell, the method, the use, or the polynucleotide vector of claim 33, wherein the one of the one or more inhibitory RNA molecules targets IFNgamma.

35. The polynucleotide of claim 1, the cell of claim 2, the method of claim 3, the use of claim 4, the polynucleotide vector of claim 5, or the polypeptide of claim 6, wherein the target antibody is structured such that binding of the anti-idiotype polypeptide to the target antibody induces cytotoxicity.

36. The method of claim 3, or the use of claim 4, further comprising instructing a user to deliver the target antibody to the subject if treatment for adverse events in the subject is warranted.

37. The modified mammalian cell of claim 2, wherein the modified mammalian cell is a CAR-T cell or a CAR-NK cell.

38. A population of modified mammalian cells of claim 2, wherein the population comprises CAR-T cells, CAR-NK cells, or both CAR-T cells and CAR-NK cells.

39. The polynucleotide, the cell, the method, the use, the polynucleotide vector, or the polypeptide of claim 23, wherein the ICD comprises an intracellular apoptosis domain capable of inducing an apoptotic signal upon binding of the anti-idiotype extracellular recognition domain to a target antibody or antibody mimetic comprising the idiotype.

40. The polynucleotide, the cell, the method, the use, the polynucleotide vector, or the polypeptide of claim 39, wherein the anti-idiotype polypeptide further comprises a proteolytic cleavage site as part of and/or between the transmembrane domain and the intracellular apoptosis domain, wherein the proteolytic cleavage site has a cleavage site amino acid sequence such that the anti-idiotype polypeptide is cleaved at the proteolytic cleavage site when the anti-idiotype polypeptide is dimerized by binding of a target antibody or antibody mimetic to the anti-idiotype extracellular recognition domain.

41. The polynucleotide, the cell, the method, the use, the polynucleotide vector, or the polypeptide of claim 40, wherein the proteolytic cleavage site is effective for cleavage by a gamma-secretase complex or a Notch receptor upon dimerization of the anti-idiotype polypeptide.

42. The polynucleotide, the cell, the method, the use, the polynucleotide vector, or the polypeptide of claim 41, wherein the intracellular apoptosis domain comprises one or more of a Caspase 2 polypeptide, a Caspase 8 polypeptide, a Caspase 9 polypeptide, and a Caspase 10 polypeptide, wherein such intracellular apoptosis domain is capable of activating an effector caspase upon cleavage from the anti-idiotype polypeptide.

43. The polynucleotide, the cell, the method, the use, the polynucleotide vector, or the polypeptide of claim 41, wherein the intracellular apoptosis domain comprises one or more caspase polypeptides, wherein the caspase polypeptides are amino acids 327 to 452 of SEQ ID NO:680 (Caspase 2), amino acids 384 to 496 of SEQ ID NO:681 (Caspase 8), amino acids 294 to 416 of SEQ ID NO:682 (Caspase 9), and amino acids 365 to 478 of SEQ ID NO:683 (Caspase 10).

44. The polynucleotide, the cell, the method, the use, the polynucleotide vector, or the polypeptide of claim 39, wherein the ICD comprises one or more caspase activation and recruitment domains (CARDs), death domains (DDs), death effector domains (DEDs), pyrin domains (PYDs), and/or caspase proteolytic domains that are activate to transmit an apoptosis-inducing signal upon dimerization.

45. The polynucleotide, the cell, the method, the use, the polynucleotide vector, or the polypeptide of claim 39, wherein the anti-idiotype polypeptide further comprises a dimerizing moiety, wherein the dimerizing moiety is constitutively dimerized, and/or wherein the target antibody is an IgA or IgM antibody.

46. The polynucleotide, the cell, the method, the use, the polynucleotide vector, or the polypeptide of claim 45, wherein the intracellular apoptosis domain comprises one or more death domains (DD) from FAS, TNF-R1, DR3, DR4, and/or DR5.

47. The vector of claim 5, wherein the vector is a viral vector, and wherein the viral vector is replication incompetent.

48. The vector of claim 47, wherein the viral vector is a retroviral particle.

49. The vector of claim 48, wherein the surface of the retroviral particle comprises a membrane-bound cytokine.

50. The vector of claim 49, wherein the membrane-bound cytokine is a chemokine.

51. The vector of claim 50, wherein the vector comprises a cleavage signal effective for cleaving the membrane-bound cytokine from the membrane.

52. The vector of claim 48, wherein the retroviral particle is a lentiviral vector.

53. The vector of claim 47, further comprising an activation element on the surface of the retroviral particle, wherein the activation element is fused to a heterologous membrane attachment sequence, and wherein the activation element is a polypeptide capable of binding to CD3 on the surface of a resting T cell and activating the resting T cell, and wherein the activation element is not encoded by a polynucleotide in the retroviral particle.

54. The vector of claim 53, wherein the activation element comprises an anti-CD3 antibody or antibody mimetic.

55. The polynucleotide of claim 1, the cell of claim 2, the method of claim 3, the use of claim 4, the polynucleotide vector of claim 5, or the polypeptide of claim 6, wherein the anti-idiotype polypeptide comprises the stalk, membrane association domain, and ICD of SEQ ID NO: 676, SEQ ID NO: 677, SEQ ID NO: 678, or SEQ ID NO: 679.

56. The polynucleotide, the cell, the method, the use, the polynucleotide vector, or the polypeptide of claim 29, wherein the transmembrane domain is the transmembrane domain of the CAR and the ICD is the ICD of a CAR.

57. The polynucleotide, the cell, the method, the use, the polynucleotide vector, or the polypeptide of claim 56, wherein the CAR is a bispecific CAR, wherein the first ASTR of the CAR comprises the anti-idiotype extracellular recognition domain.

58. The polynucleotide, the cell, the method, the use, the polynucleotide vector, or the polypeptide of claim 57, wherein the second ASTR of the CAR is capable of binding to a tumor-associated antigen or tumor-specific antigen.

59. The polynucleotide, the cell, the method, the use, the polynucleotide vector, or the polypeptide of claim 58, wherein the first ASTR and the second ASTR of the CAR are scFv antibodies.

60. The polynucleotide, the cell, the method, the use, the polynucleotide vector, or the polypeptide of claim 59, wherein the first ASTR and the second ASTR of the CAR comprise an scFv-Fc, scFv-CH, and scFv-zipper antibody.

61. A retroviral particle, comprising
a) a polynucleotide comprising nucleic acids encoding an anti-idiotype polypeptide comprising an anti-idiotype extracellular recognition domain, a membrane association domain, and a stalk that connects the anti-idiotype extracellular recognition domain to the membrane association domain, wherein the anti-idiotype extracellular recognition domain comprises an idiotype-binding variable region of an anti-idiotype antibody or antibody mimetic that recognizes the idiotype of a target antibody or a target antibody mimetic; and
b) an activation element on the surface of the retroviral particle, wherein the activation element is fused to a heterologous membrane attachment sequence, and wherein the activation element is a polypeptide capable of binding to CD3 on the surface of a T cell.

62. A method for delivering modified T cells and/or NK cells to a subject, the method comprising administering the modified cells to the subject, wherein the modified cells are modified with a polynucleotide comprising nucleic acids encoding an anti-idiotype polypeptide and nucleic acids encoding a CAR.

63. Use of modified T cells and/or NK cells in the manufacture of a kit, wherein the use of the kit comprises: administering the modified T cells and/or NK cells to a subject, wherein the modified T cells and/or NK cells comprise a polynucleotide comprising nucleic acids encoding an anti-idiotype polypeptide and nucleic acids encoding a CAR.

64. A modified primary T cell or NK cell, wherein the modified primary T cell or NK cell expresses an anti-idiotype polypeptide.

65. The modified primary T cell or NK cell of claim 63, wherein the modified primary T cell or NK cell is a tumor infiltrating lymphocyte.

66. The modified primary T cell or NK cell of claim 63, wherein the modified primary T cell or NK cell is a CAR-T or NK-T cell.

67. The modified primary T cell or NK cell of claim 63, wherein the anti-idiotype polypeptide is expressed on the surface of the T cell or NK cell.

68. A polypeptide, comprising an extracellular recognition domain, a membrane association domain, and a stalk connecting the extracellular recognition domain to the membrane association domain, wherein the extracellular recognition domain is an anti-idiotype extracellular recognition domain that recognizes an idiotype of a target antibody, and optionally wherein the anti-idiotype polypeptide is a CAR, a TCR, or a lymphoproliferative element.
